SEQUENCE LISTING (68 sequences)

SEQ ID NO: 1 - protein, 122 aa, Artificial Sequence
  MF3370-VH
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 2 - protein, 5 aa, Artificial Sequence
  MF3370-VH-CDR1
SYGIS

SEQ ID NO: 3 - protein, 17 aa, Artificial Sequence
  MF3370-VH-CDR2
WISAYNGNTNYAQKLQG

SEQ ID NO: 4 - protein, 13 aa, Artificial Sequence
  MF3370-VH-CDR3
DRHWHWWLDAFDY

SEQ ID NO: 5 - protein, 120 aa, Artificial Sequence
  MF3755-VH
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 6 - protein, 5 aa, Artificial Sequence
  MF3755-VH-CDR1
NYAMN

SEQ ID NO: 7 - protein, 17 aa, Artificial Sequence
  MF3755-VH-CDR2
WINANTGDPTYAQGFTG

SEQ ID NO: 8 - protein, 11 aa, Artificial Sequence
  MF3755-VH-CDR3
ERFLEWLHFDY

SEQ ID NO: 9 - protein, 125 aa, Artificial Sequence
  MF4280-VH
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 10 - protein, 5 aa, Artificial Sequence
  MF4280-VH-CDR1
ELSMH

SEQ ID NO: 11 - protein, 17 aa, Artificial Sequence
  MF4280-VH-CDR2
GFDPEYGKTFFAQNFQG

SEQ ID NO: 12 - protein, 16 aa, Artificial Sequence
  MF4280-VH-CDR3
EGYYETTTYYYNLFDS

SEQ ID NO: 13 - protein, 119 aa, Artificial Sequence
  MF4289-VH
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 14 - protein, 5 aa, Artificial Sequence
  MF4289-VH-CDR1
DYAMT

SEQ ID NO: 15 - protein, 17 aa, Artificial Sequence
  MF4289-VH-CDR2
WITTNTGDPTYAPGFTG

SEQ ID NO: 16 - protein, 10 aa, Artificial Sequence
  MF4289-VH-CDR3
VYHWIRGFEF

SEQ ID NO: 17 - protein, 126 aa, Artificial Sequence
  MF5790-VH
QVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 18 - protein, 7 aa, Artificial Sequence
  MF5790-VH-CDR1
SSSSYWG

SEQ ID NO: 19 - protein, 16 aa, Artificial Sequence
  MF5790-VH-CDR2
SFYYSGNTYYNPSLKS

SEQ ID NO: 20 - protein, 16 aa, Artificial Sequence
  MF5790-VH-CDR3
QTYSSSWDGVLYYFDY

SEQ ID NO: 21 - protein, 126 aa, Artificial Sequence
  MF5803-VH
QVQLQESGPGLVKPSETLSLTCTVSNGSISTYYWSWIRQPPGKGLEWIGYVYYTGRTKYNPSLKSRVTISVDTSKNQFSLNLSSVTAADTAVYYCARGGIVVVPAARDYYYYMDVWGKGTTVTVSS

SEQ ID NO: 22 - protein, 5 aa, Artificial Sequence
  MF5803-VH-CDR1
TYYWS

SEQ ID NO: 23 - protein, 16 aa, Artificial Sequence
  MF5803-VH-CDR2
YVYYTGRTKYNPSLKS

SEQ ID NO: 24 - protein, 18 aa, Artificial Sequence
  MF5803-VH-CDR3
GGIVVVPAARDYYYYMDV

SEQ ID NO: 25 - protein, 120 aa, Artificial Sequence
  MF5805-VH
EVQLVQSGAEVKKPGESLKIACKGSGFSFTSHWIGWVRQKPGRGLEWMGVIYPGDSDTRYSPSFQGQVTVSADKSINTAYLQWNSLKASDTAIYYCARPNSGSPRYFEFWGRGTLVTVSS

SEQ ID NO: 26 - protein, 5 aa, Artificial Sequence
  MF5805-VH-CDR1
SHWIG

SEQ ID NO: 27 - protein, 17 aa, Artificial Sequence
  MF5805-VH-CDR2
VIYPGDSDTRYSPSFQG

SEQ ID NO: 28 - protein, 11 aa, Artificial Sequence
  MF5805-VH-CDR3
PNSGSPRYFEF

SEQ ID NO: 29 - protein, 126 aa, Artificial Sequence
  MF5808-VH
QVQLQESGPGLVKPSETLSLTCTVSGGSISSSSYYWGWIRQPPGKGLEWIGSFYYSGNTYYNPSLKSRVTISVDTSKNQFSLKLSSVTAADTAVYYCARQEYYYGSGSPSYYFDYWGQGTLVTVSS

SEQ ID NO: 30 - protein, 7 aa, Artificial Sequence
  MF5808-VH-CDR1
SSSYYWG

SEQ ID NO: 31 - protein, 16 aa, Artificial Sequence
  MF5808-VH-CDR2
SFYYSGNTYYNPSLKS

SEQ ID NO: 32 - protein, 16 aa, Artificial Sequence
  MF5808-VH-CDR3
QEYYYGSGSPSYYFDY

SEQ ID NO: 33 - protein, 120 aa, Artificial Sequence
  MF5809-VH
EVQLVQSGAEVKKPGESLKISCKGSGDSFISHWIAWVRQMPGKGLEWMGIVYPGDSDTRYSPSFQGQVTISADKSITTAYLQWSSLKASDTAMYYCARHEWELLGPFDYWGQGTLVTVSS

SEQ ID NO: 34 - protein, 5 aa, Artificial Sequence
  MF5809-VH-CDR1
SHWIA

SEQ ID NO: 35 - protein, 17 aa, Artificial Sequence
  MF5809-VH-CDR2
IVYPGDSDTRYSPSFQG

SEQ ID NO: 36 - protein, 11 aa, Artificial Sequence
  MF5809-VH-CDR3
HEWELLGPFDY

SEQ ID NO: 37 - protein, 121 aa, Artificial Sequence
  MF5814-VH
EVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 38 - protein, 5 aa, Artificial Sequence
  MF5814-VH-CDR1
NDAIS

SEQ ID NO: 39 - protein, 17 aa, Artificial Sequence
  MF5814-VH-CDR2
SIIPILDTTDHAQKFQG

SEQ ID NO: 40 - protein, 12 aa, Artificial Sequence
  MF5814-VH-CDR3
EHIAARQDYFDY

SEQ ID NO: 41 - protein, 127 aa, Artificial Sequence
  MF5816-VH
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 42 - protein, 5 aa, Artificial Sequence
  MF5816-VH-CDR1
SYTMN

SEQ ID NO: 43 - protein, 17 aa, Artificial Sequence
  MF5816-VH-CDR2
WINTDTGDPTYAQGFTG

SEQ ID NO: 44 - protein, 18 aa, Artificial Sequence
  MF5816-VH-CDR3
GDCDSTSCYRYSYGYEDY

SEQ ID NO: 45 - protein, 119 aa, Artificial Sequence
  MF5817-VH
QVQLVQSGAEVKKPGSSVKVSCKVSGGTFRSYAISWVRQAPGQGLEWMGGIIPIFDTRNYAQILQGRVTITADLSTSTAYMELNSLRSEDTAIYYCARGSDEGDWFDPWGQGTLVTVSS

SEQ ID NO: 46 - protein, 5 aa, Artificial Sequence
  MF5817-VH-CDR1
SYAIS

SEQ ID NO: 47 - protein, 17 aa, Artificial Sequence
  MF5817-VH-CDR2
GIIPIFDTRNYAQILQG

SEQ ID NO: 48 - protein, 10 aa, Artificial Sequence
  MF5817-VH-CDR3
GSDEGDWFDP

SEQ ID NO: 49 - protein, 121 aa, Artificial Sequence
  MF5818-VH
EVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSS

SEQ ID NO: 50 - protein, 5 aa, Artificial Sequence
  MF5818-VH-CDR1
NYAIS

SEQ ID NO: 51 - protein, 17 aa, Artificial Sequence
  MF5818-VH-CDR2
SIIPILGTTDHAQKFQD

SEQ ID NO: 52 - protein, 12 aa, Artificial Sequence
  MF5818-VH-CDR3
EYIAARLDYFDS

SEQ ID NO: 53 - protein, 107 aa, Artificial Sequence
  VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 54 - protein, 6 aa, Artificial Sequence
  VL-CDR1
QSISSY

SEQ ID NO: 55 - protein, 3 aa, Artificial Sequence
  VL-CDR2
AAS

SEQ ID NO: 56 - protein, 9 aa, Artificial Sequence
  VL-CDR3
QQSYSTPPT

SEQ ID NO: 57 - protein, 107 aa, Artificial Sequence [sequence omitted]

SEQ ID NO: 58 - protein, 98 aa, Artificial Sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 59 - protein, 15 aa, Artificial Sequence
  Hinge
EPKSCDKTHTCPPCP

SEQ ID NO: 60 - protein, 110 aa, Artificial Sequence
  CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 61 - protein, 107 aa, Artificial Sequence
  CH3-DE
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 107 aa, Artificial Sequence
  CH3-KK
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 907 aa, Artificial Sequence
  MF3755x5816
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKEVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 906 aa, Artificial Sequence
  MF3755x5790
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKQVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 901 aa, Artificial Sequence
  MF3755x5818
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKEVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 911 aa, Artificial Sequence
  MF4280x5790
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKQVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 912 aa, Artificial Sequence
  MF4280x5816
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKEVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 906 aa, Artificial Sequence
  MF4280x5818
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKEVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0058444 A1
(43) Pub. Date: Feb. 22, 2024

(54) ANTIBODY COMPOSITION

(71) Applicant: MERUS N.V., Utrecht (NL)

(72) Inventors: Alexander Berthold Hendrik BAKKER, Utrecht (NL); Linda Johanna Aleida HENDRIKS, Utrecht (NL); Robert Paul DOORNBOS, Utrecht (NL); Tudor ARVINTE, Basel (CH); Guillaume Desire DARPIN, Basel (CH); Emilie Brigitte POIRIER, Basel (CH)

(21) Appl. No.: 18/268,168

(22) PCT Filed: Dec. 16, 2021
(86) PCT No.: PCT/NL2021/050772

§ 371 Date: Jun. 16, 2023

(30) Foreign Application Priority Data
Dec. 18, 2020 (NL) 2027167

(51) Int. Cl. A61K 39/395; C07K 16/28; A61K 47/22; A61K 47/12; A61K 47/26

(52) U.S. Cl. CPC A61K 39/39591; C07K 16/2863; C07K 16/28; A61K 47/22; A61K 47/12; A61K 47/26; C07K 2317/31

(57) ABSTRACT

The present invention relates to a (bio)pharmaceutical composition, particularly a liquid (e.g. aqueous) biopharmaceutical composition, more particularly a liquid biopharmaceutical composition comprising an anti-EGFR/anti-LGR5 bispecific antibody. The present invention also relates inter alia to a method of manufacturing the composition, to a kit including the composition, to a package including the composition, to a method of manufacturing the package, and to methods of treatment using the composition and/or package, especially cancer treatments, for example, for the treatment of colorectal cancer.

### INTRODUCTION

The present invention relates to a (bio)pharmaceutical composition, particularly a liquid (e.g. aqueous) biopharmaceutical composition, more particularly a liquid biopharmaceutical composition comprising an anti-EGFR/anti-LGR5 bispecific antibody. The present invention also relates inter 'alia to a method of manufacturing the composition, to a kit including the composition, to a package including the composition, to a method of manufacturing the package, and to methods of treatment using the composition and/or package, especially cancer treatments, for example, for the treatment of colorectal cancer.

### BACKGROUND

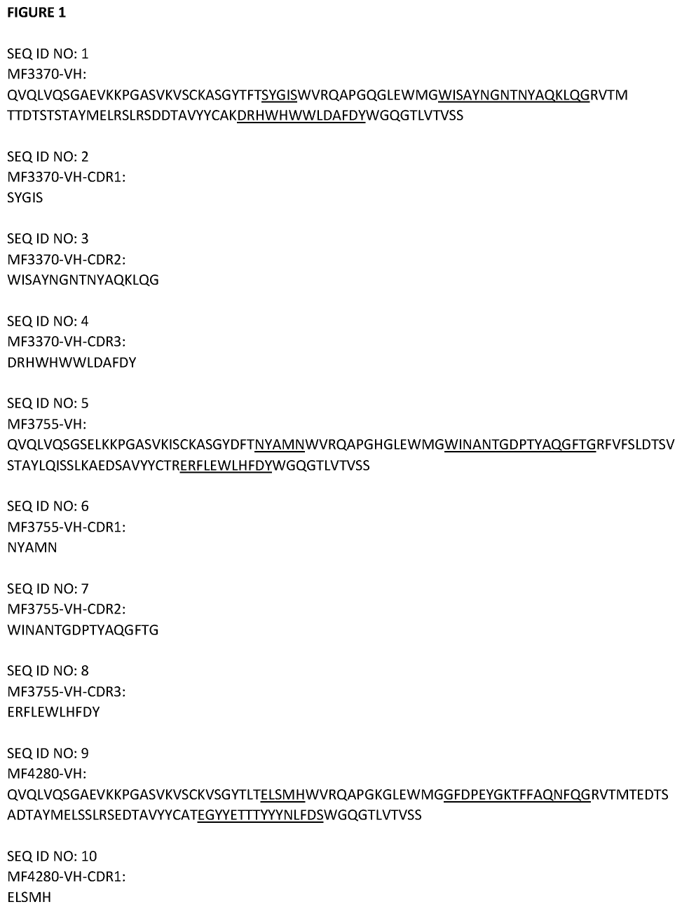

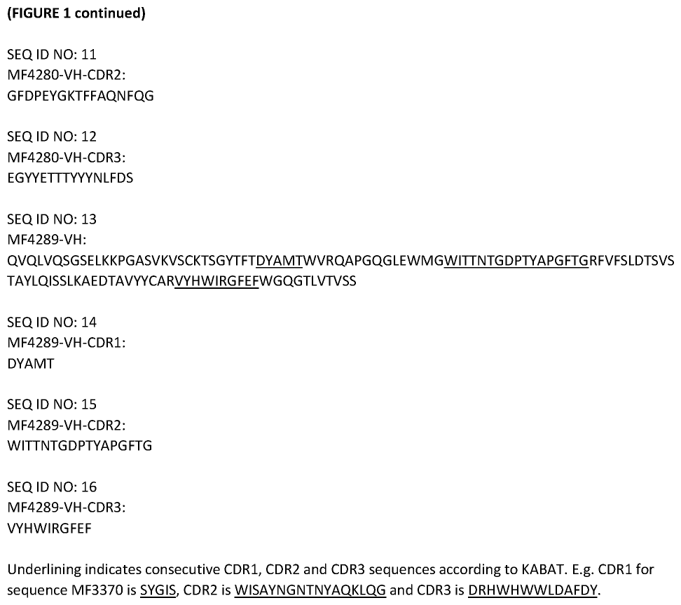

The present applicants recently developed a Biclonics® featuring antibody-dependent cell-mediated cytotoxicity (ADCC)-enhanced for the treatment of solid tumours, including the potential treatment of colorectal cancer, that binds to cancer stem cells expressing leucine-rich repeat-containing G protein-coupled receptor 5 (LGR5) and epidermal growth factor receptor (EGFR). Antibodies binding both EGFR and LGR5 are described in detail in WO2017069628A2 (MERUS et al) and relevant sequences thereof in FIGS. 1-4. Such antibodies have two different mechanisms of action: the first involves blocking growth and survival pathways in cancer stem cells; the second involves the recruitment and enhancement of immune effector cells to directly kill cancer stem cells that persist in solid tumours and cause relapse and metastasis.

An object of the present invention is to provide viable pharmaceutical compositions of a bispecific antibody binding both EGFR and LGR5 and optionally of similar variants thereof. Unpredictabilities inherent in the art of formulating biologics, especially antibodies, obstruct discovery of such viable pharmaceutical compositions, since most formulations of a given biopharmaceutical (if said formulation is arbitrarily chosen) are unstable over prolonged periods and/or under stressed conditions owing to a variety of physical or chemical factors that can affect biologics formulations, especially aqueous formulations. Preventing protein aggregation for instance can be challenging. Furthermore, a pharmaceutical composition should be formulated with the intention for it to be safely administered to human subjects, meaning the administration thereof, for instance intravenous injection, is with minimal adverse effects.

Physical or chemical influences may lead to either an unviable drug product (which may be unsafe for use in medical treatments) or a drug product whose viability is variable and unpredictable, especially in view of the variable stresses (e.g., agitation, heat, frost, light) different batches of drug product may be exposed to during manufacture, transport, and storage.

U.S. Pat. No. 6,171,586 (Lam et al.) describes stable aqueous antibody formulations. A F(ab′)2 rhuMAb CD18 antibody was formulated in sodium acetate and histidine-HCl buffers. The preferred formulation for rhuMAb CD18 was formulated in 10 mM sodium acetate, 8% trehalose, 0.01% Tween® 20, pH 5.0.

Omalizumab is a recombinant DNA-derived humanized IgG1k monoclonal antibody and available in differently formulated forms, depending on being lyophilized or liquid. In dry form, it is packed as a sterile, white, preservative-free, lyophilized powder contained in a single-use vial that is reconstituted with sterile water for injection and administered as a subcutaneous injection. One vial contains 202.5 mg of Omalizumab, 145.5 mg sucrose, 2.8 mg L-histidine hydrochloride monohydrate, 1.8 mg L-histidine and 0.5 mg polysorbate 20 and is designed to deliver 150 mg of Omalizumab, in 1.2 mL after reconstitution with 1.4 mL sterile water. In aqueous form, it is available as a pre-filled syringe. The solution for injection in the syringe contains L-arginine hydrochloride, L-histidine hydrochloride, L-histidine, polysorbate 20 and water. The label provided with the pre-filled syringe includes instructions for use, that includes storing the syringe sealed in its outer box in a refrigerator between 2° C. and 8° C. (36° F. and 46° F.) and mentions to not freeze the product. Also, a shelf life of 15 months is mentioned, including potential temperature excursions. The product may be kept for a total of 4 hours at 25° C. and if necessary, the product may be returned to the refrigerator for later use, but this must not be done more than once.

In WO2015/130173 A1, a bispecific antibody is disclosed which is formulated as a pharmaceutical composition with 25 mM Histidine, 220 mM Trehalose, 0.2 g/L PolySorbate20 or a combination thereof, with a pH set of most preferably 6.

The present invention suitably address one or more of the aforesaid stability issues and, in so doing, furnish viable pharmaceutical formulations for admistration to humans. Furnishing formulations with improved stability and/or storability is another object of the invention.

### SUMMARY OF THE INVENTION

According to an aspect of the present invention there is provided a (bio)pharmaceutical composition comprising a multispecific antibody, a functional part or derivative thereof, comprising a first binding domain binding to EGFR and a second binding domain binding to LGR5. Pharmaceutical compositions of the invention preferably comprise or consist of, some or all of the ingredients described herein (e.g. include any of a buffer, surfactant, sugar component, amino acid component, tonicifier, antioxidant or chelator), in any relevant amounts described herein, and/or may be preferably characterised by any, some, or all parameters (e.g. pH, pl, osmolality) described herein. The pharmaceutical composition is preferably a liquid (e.g. an aqueous) pharmaceutical composition.

According to an aspect of the present invention there is provided a package (e.g. vial, ampoule, syringe, pre-filled syringe, injection pen (e.g. essentially incorporating a syringe), autoinjector, or intravenous bag, or a package/container containing any of the aforementioned) comprising or containing a pharmaceutical composition as defined herein.

According to an aspect of the present invention there is provided a drug delivery device (e.g. vial, ampoule, syringe, pre-filled syringe, injection pen (e.g. essentially incorporating a syringe), autoinjector, or intravenous bag, or a package/container containing any of the aforementioned) comprising or containing a pharmaceutical composition as defined herein.

According to an aspect of the present invention there is provided a kit of parts comprising a drug delivery device, a pharmaceutical composition as defined herein (optionally contained in a package or container), and optionally a set of instructions with directions regarding the administration of the pharmaceutical composition.

According to an aspect of the present invention there is provided a method of manufacturing a pharmaceutical composition, the method comprising mixing together a multispecific antibody, a functional part, derivative or variant thereof, comprising a first binding domain binding to EGFR and a second binding domain binding to LGR5, with one or more pharmaceutically-acceptable excipients and/or carriers.

According to an aspect of the present invention there is provided a method of treating a disease or medical disorder in a patient in need of such treatment, said method comprising administering to said patient a therapeutically effective amount of a pharmaceutical composition as defined herein. Administering preferably comprises parenteral administration, which preferably includes any form of administration other than enteral and topical administration, usually by injection, but more preferably comprises intravenous delivery of a therapeutically effective amount of the pharmaceutical composition.

According to an aspect of the present invention there is provided a pharmaceutical composition, preferably as defined herein, for use in treating a disease or medical disorder in a patient in need of such treatment.

According to an aspect of the present invention there is provided a use of a pharmaceutical composition, preferably as defined herein, in the manufacture of a medicament for the treatment of a disease or disorder.

According to further aspects of the present invention, there are provided a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is an EGFR-related and LGR5-related disease. The multispecific antibody, a functional part, derivative or variant thereof, comprising a first binding domain binding to EGFR and a second binding domain binding to LGR5, preferably binds cancer stem cells expressing leucine-rich repeat-containing G-coupled receptor 5 (LGR5) and epidermal growth factor receptor (EGFR).

According to further aspects of the present invention, there are provided a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is a proliferative disease or disorder. The proliferative disease or disorder is preferably cancer. The cancer is preferably manifest in one or more solid tumours. The cancer is preferably an EGFR ligand responsive cancer, preferably that expresses a membrane associated member of the WNT pathway. The cancer is preferably an adenocarcinoma. In particular, the cancer is selected from the group consisting of: Colorectal cancer; Pancreatic cancer; Lung cancer; Breast cancer; Liver cancer; Prostate cancer; Ovarian cancer; Cervical cancer; Endometrial cancer; Head and Neck cancer; Melanoma; Testis cancer; Urothelial cancer; Renal cancer; Stomach cancer; Carcinoid cancer; and any combination thereof. Most preferably the cancer is Colorectal cancer; Pancreatic cancer; Lung cancer; Breast cancer; Liver cancer; Prostate cancer; Ovarian cancer; Cervical cancer; Endometrial cancer; Head and Neck cancer; or Melanoma. In a particularly preferred embodiment the cancer is a gastrointestinal cancer, stomach cancer or head and neck cancer. The disease or medical disorder in question may preferably be selected from any of those disclosed in WO2017069628A2, which is hereby incorporated by reference. The plausibility of treatments of the invention is furthermore justified by WO2017069628A2 in light of the present disclosure.

According to further aspects of the present invention, there are provided a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is preferably as defined herein, and wherein the treating or treatment involves combination therapy whereby the pharmaceutical composition is administered in combination with one or more other pharmaceutical or biopharmaceutical active(s); wherein said combination therapy involves simultaneous, sequential or separate dosing of the individual components of the treatment. In some embodiments, the additional pharmaceutical or biopharmaceutical active may be present within any of the pharmaceutical compositions as defined herein.

All of the aforesaid methods of treatment, composition(s) for use, and use of composition(s) in the manufacture of a medicament, are equally applicable to the relevant packages, drug-delivery devices, and kits incorporating said composition(s).

In a preferred embodiment, there is provided a pharmaceutical formulation with improved stability properties. Any features, including optional, suitable, and preferred features, described in relation to any particular aspect of the invention may also be features, including optional, suitable and preferred features, of any other aspect of the present invention.

### DETAILED DESCRIPTION OF THE INVENTION

### Definitions

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

“Antibodies”, and pertinent nomenclature such as “monoclonal”, “polyclonal”, “IgG”, “IgG1”, “IgG4”, are well known terms of art. Herein, and for the avoidance of doubt, the term “antibody” means a proteinaceous molecule belonging to the immunoglobulin class of proteins, containing one or more domains that bind an epitope on an antigen, where such domains are or are derived from or share sequence homology with the variable domain of an antibody. Antibodies are typically made of basic structural units—each with two heavy chains and two light chains. Antibodies for therapeutic use are preferably as close to natural antibodies of the subject to be treated as possible (for instance human antibodies for human subjects).

The term “binding domain” as used herein means a part of an antibody that functions in antigen binding comprising a variable domain or that shares sequence homology with a variable domain. Non-limiting examples of binding domains comprising a variable domain are an Fv domain and a Fab domain. Typical variability is found in three superficial-loop forming regions in the VH and VL domains, which are the complementarity determining regions or CDRs.

The term “heavy chain” or “immunoglobulin heavy chain” includes an immunoglobulin heavy chain constant region sequence from any organism, and unless otherwise specified includes a heavy chain variable domain. The term heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen and that comprises at least one CDR. Herein, CDR domains for the heavy chain preferably follow the Kabat numbering system

The term “light chain” includes an immunoglobulin light chain variable domain, or VL (or functional fragment thereof); and an immunoglobulin constant domain, or CL (or functional fragment thereof) sequence from any organism.

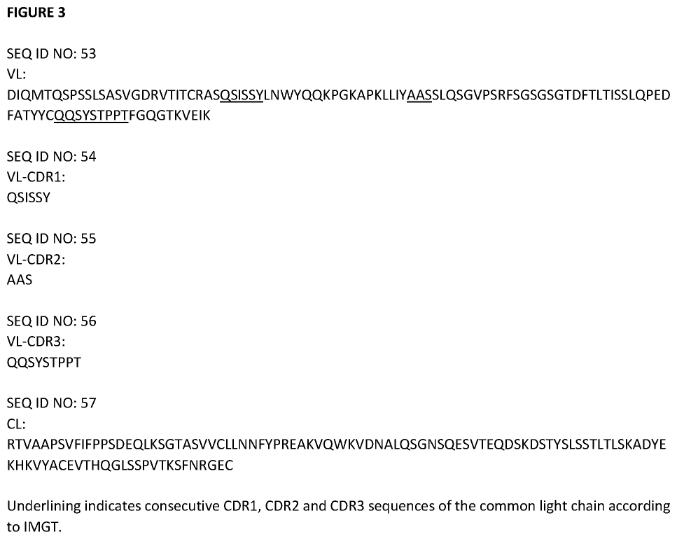

Unless otherwise specified, the term light chain may include a light chain selected from a human kappa, lambda, and a combination thereof. Light chain variable (VL) domains typically include three light chain CDRs and four framework (FR) regions, unless otherwise specified. Generally, a full-length light chain includes, from N-terminus to C-terminus, a VL domain that includes FR1-CDR1-FR2-CDR2-FR3-CDR3-FR4, and a light chain constant domain. Light chains that can be used with this invention include those, e.g., that do not selectively bind an epitope selectively bound by the heavy chains. An example of such a light chain is the light chain presented in FIG. 3. Herein, CDR domains for the light chain preferably follow the IMGT numbering system.

Suitable light chains for use in an antibody of the invention include a common light chain, such as those that can be identified by screening for the most commonly employed light chains in existing antibody libraries (wet libraries or in silico), where the light chains do not substantially interfere with the affinity and/or selectivity of the epitope-binding domains of the heavy chains, but are also suitable to pair with an array of heavy chains. For example, a suitable light chain includes one from a transgenic animal, such as a transgenic rodent, comprising the common light chain integrated into its genome and which can be used to generate large panels of common light chain antibodies having diversity at the heavy chain upon exposure to an antigen (WO2009/157771). Relevant sequences of a light chain, in particular a CL, VL, VL-CDR1, VL-CDR2 and VL-CDR3, of the invention are given in FIG. 3.

The term “common light chain” according to the invention refers to light chains which may be identical or have some amino acid sequence differences while the binding specificity of the an antibody of the invention is not affected, i.e. the differences do not materially influence the formation of functional binding regions.

It is for instance possible within the scope of the definition of common chains as used herein, to prepare or find variable chains that are not identical but still functionally equivalent, e.g., by introducing and testing conservative amino acid changes, changes of amino acids in regions that do not or only partly contribute to binding specificity when paired with a cognate chain, and the like. Such variants are thus also capable of binding different cognate chains and forming functional antigen binding domains. The term ‘common light chain’ as used herein thus refers to light chains which may be identical or have some amino acid sequence differences while retaining the binding specificity of the resulting antibody after pairing with a heavy chain. A combination of a certain common light chain and such functionally equivalent variants is encompassed within the term “common light chain”.

Those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions.

The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen binding specificity of the IgG.

A “bispecific antibody” as meant herein is an antibody wherein one variable domain of the antibody binds to a first antigen whereas a second variable domain of the antibody binds to a second antigen, wherein said first and second antigens are not identical. The term includes antibodies wherein at least one VH is capable of specifically recognizing a first antigen and a VL, paired with the at least one VH in an immunoglobulin variable domain, is capable of specifically recognizing a second antigen. The resulting VH/VL pair will bind either antigen 1 or antigen 2, and are called “two-in-one antibodies”, described in for instance WO 2008/027236, WO 2010/108127 and Schaefer et al (Cancer Cell 20, 472-486, October 2011).

A bispecific antibody according to the present invention is preferably a human IgG, more preferably a human IgG1. Preferably, a bispecific antibody as meant herein has binding specificity for LGR5 and EGFR and does not contain further variable domains.

A “multispecific antibody” as meant herein, such as a bispecific or trispecific antibody, is an antibody wherein one variable domain of the antibody binds to a first antigen, a second variable domain of the antibody binds to a second antigen, and in case of a trispecific antibody a third variable domain of the antibody binds to a third antigen, wherein said first, second and third antigens are not identical or epitopes to which they bind are not identical.

The “antibody comprising a first binding domain binding to EGFR and a second binding domain binding to LGR5” as meant herein is a multispecific antibody, preferably a bispecific antibody (BsAb). It is also referred to herein as “anti-EGFR/anti-LGR5 bispecific antibody” or simply “EGFR/LGR5 BsAb”. The antibody, and its production, is described in detail in WO2017069628A2 (MERUS et al). The antibody is preferably a full-length, human IgG1 heterodimer having an anti-EGFR portion and an anti-LGR5 portion; relevant DNA and/or amino acid sequences of which are disclosed in FIGS. 1-4 of WO2017069628A2 and mentioned herein. The molecular weight is approximately 146 kDa, and this molecular weight is used for the purposes of any molar calculations. As such, a 10 mg/mL aqueous solution of antibody corresponds to a 0.068 mM solution; a 20 mg/mL aqueous solution corresponds to a 0.137 mM antibody solution; and a 50 mg/mL aqueous antibody solution corresponds to a 0.342 mM solution thereof.

A “functional part” of a bispecific antibody comprises the antigen binding parts of the bispecific antibody or a derivative of the binding parts. The binding part of an antibody is encompassed in the variable domain.

A “derivative” of an antibody is a protein that but for the CDR regions deviates from the amino acid sequence of a natural antibody in at most 20 amino acids. A derivative of an antibody as disclosed herein is an antibody that deviates from said amino acid sequence in at most 20 amino acids.

With the term “EGFR” is herein meant the epidermal growth factor receptor, which is a member of the family of four receptor tyrosine kinases (RTKs). EGFR is known under various synonyms, such as HER1 (Human Epidermal growth factor (EGF) Receptor 1) and ErbB1 (Erythroblastoma-1). EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. The major signal transduction pathway activated by EGFR is composed of the Ras-mitogen-activated protein kinase (MAPK) mitogenic signalling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients.

The database accession number for the human EGFR protein and the gene encoding it is (GenBank NM_005228.3). This accession number is primarily given to provide a further method of identification of EGFR protein as a target, the actual sequence of the EGFR protein bound by an antibody may vary, for instance because of a mutation in the encoding gene such as those occurring in some cancers or the like. The words cancer and tumor are used herein and typically both refer to cancer, unless otherwise specifically stated. Where reference herein is made to EGFR, the reference refers to human EGFR unless otherwise stated. The antigen-binding site that binds EGFR, binds EGFR and a variety of variants thereof such as those expressed on some EGFR positive tumors.

The term “LGR” refers to the family of proteins known as Leucine-rich repeat-containing G-protein coupled receptors. Among the members of this family is LGR5 which is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so.

Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

References herein to any particular bispecific antibodies, including as disclosed in WO2017069628A2, include the relevant originator drug substance, whether as commercially available, as described in a patent document, or as described elsewhere in the art.

The term sequence identity is well known in the art. Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI 30 Advance® 11.5.2 software using the default settings, which employ a modified ClustalW algorithm (Thompson, J. D., Higgins, D. G., and Gibson T. J., (1994) Nuc. Acid Res. 22(22): 4673-4680), the swgapdnamt score matrix, a gap opening penalty of 15 and a gap extension penalty of 6.66. Amino acid sequences are aligned with the AlignX application of the Vector NTI Advance® 11.5.2 software using default 35 settings, which employ a modified ClustalW algorithm (Thompson, J. D., Higgins, D. G., and Gibson T. J., (1994) Nuc. Acid Res. 22(22): 4673-4680), the blosum62mt2 score matrix, a gap opening penalty of 10 and a gap extension penalty of 0.1.

Herein, the term “buffer”, “buffer system”, or “buffered solution” refers to a generally aqueous solution comprising a mixture of an acid (usually a weak acid, e.g. acetic acid, citric acid, imidazolium form of histidine) and its conjugate base (e.g. an acetate or citrate salt, for example, sodium acetate, sodium citrate, or histidine) or alternatively a mixture of a base (usually a weak base, e.g. histidine) and its conjugate acid (e.g. protonated histidine salt). The pH of a “buffered solution” will change only slightly upon addition of a small quantity of strong acid or base due to the “buffering effect” imparted by the buffer system.

Herein, a “buffer system” comprises one or more buffering agent(s) and/or an acid/base conjugate(s) thereof, and more suitably comprises one or more buffering agent(s) and an acid/base conjugate(s) thereof, and most suitably comprises one buffering agent only and an acid/base conjugate thereof. Unless stated otherwise, any concentrations stipulated herein in relation to a “buffer system” (i.e. a buffer concentration) suitably refers to the combined concentration of the buffering agent(s) and/or acid/base conjugate(s) thereof. In other words, concentrations stipulated herein in relation to a “buffer system” suitably refer to the combined concentration of all the relevant buffering species (i.e. the species in dynamic equilibrium with one another, e.g. acetate/acetic acid). As such, a given concentration of an acetate buffer system generally relates to the combined concentration of acetate (or acetate salt(s), e.g. sodium acetate) and acetic acid. The overall pH of the composition comprising the relevant buffer system is generally a reflection of the equilibrium concentration of each of the relevant buffering species (i.e. the balance of buffering agent(s) to acid/base conjugate(s) thereof). Any given “buffer system” may consist of a single buffer (or buffer system) or of a plurality of buffers (or buffer systems).

Herein, the term “buffering agent” refers to an acid or base component (usually a weak acid or weak base) of a buffer or buffer solution. A buffering agent helps maintain the pH of a given solution at or near to a pre-determined value, and the buffering agents are generally chosen to complement the pre-determined value. A buffering agent is suitably a single compound which gives rise to a desired buffering effect, especially when said buffering agent is mixed with (and suitably capable of proton exchange with) an appropriate amount (depending on the pre-determined pH desired) of its corresponding “acid/base conjugate”, or if the required amount of its corresponding “acid/base conjugate” is formed in situ—this may be achieved by adding strong acid or base until the required pH is reached. By way of example:

An “acetate buffering agent” is suitably an acetate salt, for example, sodium acetate, suitably mixed with its acid/base conjugate, acetic acid. Such a buffer system may be formed by simply mixing a given amount of sodium acetate with a given amount of acetic acid. Alternatively, however, such a buffer may be formed by adding a given amount of a base, suitably a strong base (e.g. sodium hydroxide) to the acetic acid until the desired pH (and thus the desired balance of sodium acetate/acetic acid) is reached. Herein, except where the contrary is stated, any concentrations given in relation to an acetate buffer or acetate buffering agent suitably refer to the combined concentration of the buffering agent(s) (e.g. sodium acetate) and/or acid/base conjugate(s) thereof (e.g. acetic acid). The skilled person is readily able to calculate such concentrations. Such concentrations may be calculated by reference to the combined concentrations of buffering agent(s) and acid/base conjugate(s), where a buffer system is formed by simply mixing together buffering agent(s) and acid/base conjugate(s). Alternatively, where a buffer system is formed by mixing either the buffering agent(s) or acid/base conjugate(s) with a pH adjuster (e.g. strong acid or strong base) to produce a mixture of each, suitably such concentrations may be calculated by reference to the starting amounts/concentrations of the buffering agent(s) or acid/base conjugate(s) respectively. For example, where a buffer system is formed using a known amount/concentration of acetic acid which is mixed with a pH adjuster (e.g. sodium hydroxide) until the desired pH is reached, the concentration of the buffer system may be calculated by reference to the initial amount of acetic acid.

Herein, an “acid/base conjugate” refers to the conjugate acid or conjugate base (whichever is relevant at a particular pH—typically the conjugate acid in the context of the present invention) of a particular “buffering agent”. The acid/base conjugate of an acetate buffering agent (e.g. sodium acetate) is suitably acetic acid.

Herein, the term “buffering species” refers to the particular species (excluding any associated counteranions or countercations, ignoring sodium ions for sodium acetate/acetic acid systems) of a given buffer system which are in dynamic equilibrium with (and proton-exchange with) one another. For example, acetate anions and acetic acid together constitute the “acetate buffering species” of a “acetate buffer system”.

Since it is impractical to define quantities (whether absolute or relative) of a buffer system by reference to weight (since the total weight will depend on the desired pH, which will affect the amount of counterions present), herein weight-based quantities are instead determined by reference to a theoretical weight of the relevant “buffering species”. At least two species are present in any given set of “buffering species” (in relative amounts that can only be determined by reference to the pH), each with a different molecular weight (which usually differs by just 1). Therefore, to enable viable weight calculations and references, for the purposes of this specification the weight of any given set of “buffering species” is given as a theoretical weight based on just one of the buffering species, namely the most acidic of the buffering species (i.e. the most protonated form at any given pH). So the weight of a given set of “buffering species” is quoted as the weight of acid-species equivalents. By way of example, in an acetate buffer system the acetate buffering species may consist of acetate anions (ignore countercations) and acetic acid. The weight of the “buffering species” is therefore calculated as if acetic acid was the only species present in the buffer system (even though acetate is clearly present alongside acetic acid). Thus, any reference to a weight or weight ratio involving a “acetate buffering species” suitably refers to the theoretical weight of acetic acid equivalents within the buffer system. As such, where a composition is formed by adding a pH adjuster (e.g. sodium hydroxide) to a fixed amount of acetic acid, the original weight of acetic acid may be considered to be the weight of the “buffering species” regardless of the ultimate pH. Alternatively, if the concentration (i.e. molarity) of a buffer system is known, this can be converted into a weight of “buffering species” by reference to the molecular weight of the most acidic form of the relevant buffering species (e.g. acetic acid), and ignoring the fact that acetate anions are also present.

Herein, any references to a ‘buffer system’ may suitably be construed as a reference to a ‘buffer’ and the term ‘buffer’ may replace all instances of the term ‘buffer system’—e.g. an ‘acetate buffer system’ is suitably construed as an ‘acetate buffer’. As explained in relation to the term ‘buffer system’, a particular ‘buffer’ generally includes an equilibrium mixture of conjugate acid(s) and conjugate base(s)—e.g. an ‘acetate buffer’ includes acetate anions (e.g. an acetate salt) and acetic acid; a ‘histidine buffer’ generally includes free histidine (i.e. unprotonated, neutral histidine) and the imidazolium (protonated) form of histidine. As explained in relation to the term ‘buffer system’, the concentration of a particular ‘buffer’ suitably means the combined concentrations of all relevant conjugate acid(s) and conjugate base(s). For instance, the concentration of said ‘acetate buffer’ suitably refers to the combined concentration of acetate anions and acetic acid; the concentration of said ‘histidine buffer’ suitably refers to the combined concentration of free histidine and the imidazolium form of histidine (e.g. concentration of histidine+concentration of histidine.HCl). As explained in relation to ‘buffer system’, a ‘buffer’ may consist of a single ‘buffer’ or a plurality of ‘buffers’. In this regard, references herein to ‘buffer systems’ may suitably be construed as a reference to ‘buffers’ and the term ‘buffers’ may replace all instances of the term ‘buffer systems’. Where a buffer comprises a plurality of buffers, the concentration of said plurality of buffers is suitably the combined concentration of said plurality of buffers (i.e. the combined concentration of all relevant conjugate acid(s) and conjugate base(s)). For instance, a buffer (or ‘buffer system’) comprising (or consisting of) both an acetate buffer and a histidine buffer suitably contains a combination of acetate anions and acetic acid (forming the acetate buffer) and also a combination of free histidine and the imidazolium (protonated) form of histidine (forming the histidine buffer), and the total concentration of the buffer may be the sum total of acetate anions, acetic acid, free histidine, and imidazolium form of histidine.

Suitably, the ‘buffer system’ or ‘buffer’ is a component of a composition rather than the entire composition itself. As such, suitably a ‘buffered composition’ or ‘buffered solution’ would comprise a ‘buffer system’ or ‘buffer’.

Herein, in the context of the present specification, a “strong acid” is suitably one having a pKₐ of −1.0 or less, whereas a “weak acid” is suitably one having a pKₐ of 2.0 or more. Herein, in the context of the present specification, a “strong base” is suitably one whose conjugate acid has a pKₐ of 12 or higher (suitably 14 or higher), whereas a “weak base” is suitably one whose conjugate acid has a pKₐ of 10 or less.

A person skilled in the art will be aware of the well-known “Henderson-Hasselbalch equation”, which in the context of buffer systems and compositions comprising buffer systems links pH (including pH of an overall composition) with the relative concentrations of relevant conjugate acid and conjugate base and the pKₐ of the conjugate acid. The equation in question is:

$$pH = {pK}_{a} + {log}_{10} \frac{[ Base ]}{[ Acid ]}$$

The pKₐ of various conjugate acids is well documented. For instance, the conjugate acid (protonated form) of histidine (i.e. the imidazolium form of histidine) has a pKₐ of about 6. From this it is possible either to determine a pH from known input concentrations of free histidine and imidazolium histidine (conjugate base and conjugate acid respectively) or to determine the prevailing relative concentrations of free histidine and imidazolium histidine in a histidine-buffered solution exhibiting a known pH.

Unless stated otherwise, references herein to a “pKₐ” should be construed as a pKₐ value in water at standard ambient temperature and pressure (SATP), suitably of the conjugate acid of the relevant species.

An “amino acid component” is suitably an ingredient or ingredients comprising one or more amino acids, though an amino acid component preferably consist of a single amino acid. For sake of clarity, it is emphasized that an amino acid component as meant herein is added to the pharmaceutical formulation with the intention to provide a buffering function. As such, the amino acid component does not include any protein, antibody or active pharmaceutical ingredient.

Herein, a “sugar component” is suitably an ingredient or ingredients comprising one or more sugar(s) and/or sugar alcohol(s), though a sugar component may consist of a single sugar or sugar alcohol, and excludes sugar or glycosylation of an antibody or active pharmaceutical ingredient.

Herein, a “non-reducing sugar” is generally a sugar without any aldehyde moieties or without the capability of forming an aldehyde moiety (e.g. through isomerism).

Herein, a “tonicity modifier” or “tonicifier” refers to a reagent whose inclusion within a composition suitably contributes to (or increases) the overall osmolality and osmolarity of the composition. Suitably, a tonicifier, as used herein includes an agent which functions to render a solution similar in osmotic characteristics to physiologic fluids.

Herein, an “antioxidant” or “antioxidant component” is suitably an ingredient or ingredients comprising one or more antioxidant compounds, though an antioxidant component may consist of a single antioxidant compound. An antioxidant in the context of the compositions of the invention suitably mitigate oxidation of groups within the fusion protein that might otherwise be vulnerable to oxidation.

“Chelator” is a term of art referring to a compound capable of complexing, suitably in a multidentate manner, with various groups, molecules, atoms, or ions, and may exert an antioxidant effect in its own right.

Herein, wherever a composition is said “to be characterised by an absence of [a particular component]”, this suitably means that the composition in question is either substantially free or entirely free of said component.

The term “substantially free”, when used in relation to a given component of a composition (e.g. “a liquid pharmaceutical composition substantially free of an amino acid component”), refers to a composition to which essentially none of said component has been added. As explained above, such references have no bearing on the presence of amino acid residue(s) within a protein structure. When a composition is “substantially free” of a given component, said composition suitably comprises no more than 0.1 wt % of said component, suitably no more than 0.01 wt % of said component, suitably no more than 0.001 wt % of said component, suitably no more than 0.0001 wt % of said component, suitably no more than 0.00001 wt %, suitably no more than 0.000001 wt %, suitably no more than 0.0000001 wt % thereof, most suitably no more than 0.0001 parts per billion (by weight).

The term “entirely free”, when used in relation to a given component of a composition (e.g. “a liquid pharmaceutical composition substantially free of an amino acid component”), refers to a composition containing none of said component. As explained above, such references have no bearing on the presence of amino acid residue(s) within a protein structure.

Suitably, unless stated otherwise, where reference is made to a parameter (e.g. pH, pKa, etc.) or state of a material (e.g. liquid, gas, etc.) which may depend on pressure and/or temperature, suitably in the absence of further clarification such a reference refers to said parameter at standard laboratory temperature and pressure conditions.

Herein, references to specific amounts of a given component of a composition, especially a buffering agent or buffer system, surfactant, sugar component, amino acid component, tonicifier, antioxidant, and/or chelator suitably relate to the amounts of the pure anhydrous form of the relevant component (or compositions formed by using said amounts of the pure anhydrous form), even though such a component may be used in a non-anhydrous form when forming the composition.

Amounts of any corresponding non-anhydrous forms (e.g. monohydrates, dihydrates, etc.) may be readily calculated by simply using the appropriate multiplier. For instance, unless stated otherwise (as per the Examples, where quantities relate to trehalose dihydrate), amounts stipulated in relation to trehalose refer to the anhydrous form of trehalose (or compositions formed by using the stipulated amounts/concentrations of anhydrous trehalose), which has a molecular weight of 342.296 g/mol, so to calculate the corresponding amount of trehalose dihydrate needed to form the same composition (less water would have to be added) it is necessary to multiply the stipulated amount by 378.33/342.296, since 378.33 is the molecular weight of trehalose dihydrate. The skilled person would readily understand how to judiciously adjust the quantity of diluent/water depending on the form of the components used, in order to derive the target concentrations. Where molar quantities are stipulated this issue does not apply.

Herein, the term “pharmaceutical composition” or “biopharmaceutical composition” refers to a formulation of a (bio)pharmaceutical active which renders the biological activity of the active ingredient therapeutically effective, but which does not include other ingredients which are obviously toxic to a subject to which the formulation are intended to be administered. Herein, references to a “composition” generally refer to a (bio)pharmaceutical composition as defined herein.

Herein, the term “stable” generally refers to the physical stability and/or chemical stability and/or biological stability of a component, typically an active or composition thereof, during preservation/storage. For aqueous compositions of a biologic, storage stability may suitably mean that the biologic is sufficiently stable (i.e. with prescribed limits for patient safety) when stored at 2-8° C. for at least 6 months, suitably at least 12 months, suitably up to 24 months or longer. However, accelerated stability studies may be used to provide relevant stability information.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, an effective amount is an amount sufficient to delay tumor development. In some embodiments, an effective amount is an amoun sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of an EGFR/LGR5 antibody and a topoisomerase I inhibitor, in combination, to effect a decrease in a cancer (for example a decrease in the number of cancer cells); slowing of progression of a cancer, or prevent regrowth or recurrence of the cancer, wherein the cancer is gastrointestinal cancer, preferably colorectal cancer.

Herein, amounts stipulated for components and ingredients, whether specified in terms of “parts”, ppm (parts per million), percentages (%, e.g. wt %), or ratios, are intended to be by weight, unless stated otherwise.

Where the quantity or concentration of a particular component of a given composition is specified as a weight percentage (wt % or % w/w), said weight percentage refers to the percentage of said component by weight relative to the total weight of the composition as a whole. It will be understood by those skilled in the art that the sum of weight percentages of all components of a composition (whether or not specified) will total 100 wt %. However, where not all components are listed (e.g. where compositions are said to “comprise” one or more particular components), the weight percentage balance may optionally be made up to 100 wt % by unspecified ingredients (e.g. a diluent, such as water, or other non-essentially but suitable additives).

Where a composition is said to comprise a plurality of stipulated ingredients (optionally in stipulated amounts of concentrations), said composition may optionally include additional ingredients other than those stipulated. However, in certain embodiments, a composition said to comprise a plurality of stipulated ingredients may in fact consist essentially of or consist of all the stipulated ingredients, optionally in the amounts specified—where compositions are aqueous compositions, water is an implicit ingredient even if not stated. In either circumstance, an individual component may itself comprise, consist essentially of, or consist of a sub-component or one or more sub-components. Herein, wherever the term “comprise” is used it may, where compatible with the context, be replaced by “consists essentially of” or “consists of”.

Herein, where a composition is said to “consists essentially of” a particular component, said composition suitably comprises at least 70 wt % of said component, suitably at least 90 wt % thereof, suitably at least 95 wt % thereof, most suitably at least 99 wt % thereof. Suitably, a composition said to “consist essentially of” a particular component consists of said component save for one or more trace impurities.

Herein, unless incompatible in a given context, wherever a component is stipulated which is capable of ionization (e.g. protonation or deprotonation), the definition of said component suitably includes any suitable salts thereof, suitably pharmaceutically acceptable salts thereof. For example, this applies to any references herein to buffering agents (e.g. citric acid or citrate), amino acids, and such like. Likewise, unless incompatible in a given context, wherever a component is stipulated which is capable of neutralisation, the definition of said component suitably includes neutralised forms thereof—e.g. citric acid instead of citrate.

### Pharmaceutical Composition

The present invention provides a pharmaceutical composition, in particular a pharmaceutical composition comprising a multispecific antibody, functional part or derivative thereof, comprising a first binding domain binding to EGFR and a second binding domain binding to LGR5; a buffer system comprising a histidine buffer and/or a citrate buffer; a sugar component; and a non-ionic surfactant.

The composition is most preferably a liquid pharmaceutical composition, preferably an aqueous pharmaceutical composition. Generally, amounts and concentrations herein relate to liquid pharmaceutical compositions, though such amounts may be readily converted into corresponding amounts within a corresponding solid composition for sake of calculation. As such, amounts may be expressed as (weight or molar) ratios between the respective components or as absolute concentrations (by taking account of the removal of the diluent).

The composition preferably comprises a multispecific or bispecific antibody comprising a first binding domain binding to EGFR and a second binding domain binding to LGR5 (i.e. an EGFR/LGR5 BsAb), sometimes referred to herein as simply “the antibody”. The composition preferably comprises a diluent. The composition preferably comprises a buffer system, preferably a histidine or citrate buffer system, more preferably a histidine buffer system. The composition preferably comprises a salt tonicifier. The composition preferably comprises a sugar component, the sugar component preferably is or comprises sucrose. The composition preferably comprises a surfactant, more preferably a non-ionic surfactant which is or comprises polysorbate 80. The composition preferably comprises an amino acid component.

The pH of the pharmaceutical composition preferably is between 5-7, preferably 5.4-6.5, more preferably 5.8-6.4, most preferably 5.9 or 6.3.

The antibody is present in the pharmaceutical composition of the invention is present at a concentration of 0.5-150 mg/mL, preferably 1-100 mg/mL, more preferably 1 to 50 mg/mL, more preferably 1 to 30 mg/mL, more preferably 5-25 mg/mL, more preferably 15-25 mg/mL, most preferably 20 mg/mL.

The sugar component is preferably present at a concentration of 200-400 mM, preferably 250-350, more preferably 270 to 300 mM, most preferably 280 mM or 290 mM.

The non-ionic surfactant is preferably present at a concentration of 0.01-2 mg/mL, preferably 0.1-1.5 mg/mL, more preferably 0.1-0.6 mg/mL or 0.6-1.2 mg/mL, most preferably 0.2 mg/mL, 0.5 mg/mL or 1 mg/mL.

The buffer system, or combination of buffer systems, is preferably present at a concentration between 2 and 50 mM, preferably 3 to 20 mM, more preferably 4 to 12 mM, more preferably 3-7 mM or 8 to 12 mM, most preferably 5 mM or 10 mM.

The molar concentration ratio of sugar component to antibody is preferably between 250:1 and 60,000:1, preferably 500:1 and 5000:1, preferably 1460:1 and 2920:1, preferably 1825:1 and 2555:1, more preferably 1970:1 and 2190:1, most preferably about 2040:1 or 2117:1.

The molar concentration ratio of non-ionic surfactant to antibody is preferably between 1:90 and 224:1, preferably 1:50 and 20:1, preferably 1:18 and 11:1, preferably 1:2 and 8.5:1, more preferably 1:2 and 3.5:1 or 3.5:1 and 7:1, most preferably about 1.11:1, about 2.79:1, or about 5.55:1.

The molar concentration ratio of buffer system (or combination of buffer systems) to antibody is preferably between 2.9:1 and 7300:1, preferably 6:1 and 700:1, preferably 14:1 and 365:1, preferably 21:1 and 146:1, preferably 29:1 and 88:1, more preferably 21:1 and 51:1 or 58:1 and 88:1, most preferably about 36.5:1 or 73:1.

The buffer system preferably comprises or consists of a citrate buffer system.

The composition preferably comprises only a single buffer system, such as a histidine or a citrate buffer.

Preferably the composition is free of trehalose and/or free of polysorbate 20 and/or non-buffering salt tonicifiers, most preferably free of sodium chloride.

The composition is preferably free of antioxidants and/or free of chelators.

The composition may preferably be characterised by an absence of water or less than 5 wt % water. The composition may suitably be characterised by an absence of a (non-buffering) salt tonicifier. The composition may suitably be characterised by an absence of an antioxidant (or of any one or more of those specifically mentioned herein in relation to the antioxidant). The composition may suitably be characterised by an absence of a chelator (or of any one or more of those specifically mentioned herein in relation to the chelator).

### A) Embodiments of Combinations of Ingredients for Inclusion

The pharmaceutical composition comprises the antibody, a functional part, derivative or variant thereof and optionally one or more pharmaceutically acceptable excipients and/or carriers.

The following numbered paragraphs A1 to A127 disclose specific embodiments of the invention, in which the pharmaceutical composition comprises (or consists of, optionally along with water for injection in the case of aqueous compositions) and is characterised by:
A1. an EGFR/LGR5 BsAb; and a buffer system.
A2. an EGFR/LGR5 BsAb; and a salt tonicifier.
A3. an EGFR/LGR5 BsAb; and a sugar component.
A4. an EGFR/LGR5 BsAb; and a surfactant.
A5. an EGFR/LGR5 BsAb; and an amino acid component.
A6. an EGFR/LGR5 BsAb; and an antioxidant.
A7. an EGFR/LGR5 BsAb; and a chelator.
A8. an EGFR/LGR5 BsAb; a buffer system; and a salt tonicifier.
A9. an EGFR/LGR5 BsAb; a buffer system; and a sugar component.
A10. an EGFR/LGR5 BsAb; a buffer system; and a surfactant.
A11. an EGFR/LGR5 BsAb; a buffer system; and an amino acid component.
A12. an EGFR/LGR5 BsAb; a buffer system; and an antioxidant.
A13. an EGFR/LGR5 BsAb; a buffer system; and a chelator.
A14. an EGFR/LGR5 BsAb; a salt tonicifier; and a sugar component.
A15. an EGFR/LGR5 BsAb; a salt tonicifier; and a surfactant.
A16. an EGFR/LGR5 BsAb; a salt tonicifier; and an amino acid component.
A17. an EGFR/LGR5 BsAb; a salt tonicifier; and an antioxidant.
A18. an EGFR/LGR5 BsAb; a salt tonicifier; and a chelator.
A19. an EGFR/LGR5 BsAb; a sugar component; and a surfactant.
A20. an EGFR/LGR5 BsAb; a sugar component; and an amino acid component.
A21. an EGFR/LGR5 BsAb; a sugar component; and an antioxidant.
A22. an EGFR/LGR5 BsAb; a sugar component; and a chelator.
A23. an EGFR/LGR5 BsAb; a surfactant; and an amino acid component.
A24. an EGFR/LGR5 BsAb; a surfactant; and an antioxidant.
A25. an EGFR/LGR5 BsAb; a surfactant; and a chelator.
A26. an EGFR/LGR5 BsAb; an amino acid component; and an antioxidant.
A27. an EGFR/LGR5 BsAb; an amino acid component; and a chelator.
A28. an EGFR/LGR5 BsAb; an antioxidant; and a chelator.
A29. an EGFR/LGR5 BsAb; a buffer system; a salt tonicifier; and a sugar component.
A30. an EGFR/LGR5 BsAb; a buffer system; a salt tonicifier; and a surfactant.
A31. an EGFR/LGR5 BsAb; a buffer system; a salt tonicifier; and an amino acid component.
A32. an EGFR/LGR5 BsAb; a buffer system; a salt tonicifier; and an antioxidant.
A33. an EGFR/LGR5 BsAb; a buffer system; a salt tonicifier; and a chelator.
A34. an EGFR/LGR5 BsAb; a buffer system; a sugar component; and a surfactant.
A35. an EGFR/LGR5 BsAb; a buffer system; a sugar component; and an amino acid component.
A36. an EGFR/LGR5 BsAb; a buffer system; a sugar component; and an antioxidant.
A37. an EGFR/LGR5 BsAb; a buffer system; a sugar component; and a chelator.
A38. an EGFR/LGR5 BsAb; a buffer system; a surfactant; and an amino acid component.
A39. an EGFR/LGR5 BsAb; a buffer system; a surfactant; and an antioxidant.
A40. an EGFR/LGR5 BsAb; a buffer system; a surfactant; and a chelator.
A41. an EGFR/LGR5 BsAb; a buffer system; an amino acid component; and an antioxidant.
A42. an EGFR/LGR5 BsAb; a buffer system; an amino acid component; and a chelator.
A43. an EGFR/LGR5 BsAb; a buffer system; an antioxidant; and a chelator.
A44. an EGFR/LGR5 BsAb; a salt tonicifier; a sugar component; and a surfactant.
A45. an EGFR/LGR5 BsAb; a salt tonicifier; a sugar component; and an amino acid component.
A46. an EGFR/LGR5 BsAb; a salt tonicifier; a sugar component; and an antioxidant.
A47. an EGFR/LGR5 BsAb; a salt tonicifier; a sugar component; and a chelator.
A48. an EGFR/LGR5 BsAb; a salt tonicifier; a surfactant; and an amino acid component.
A49. an EGFR/LGR5 BsAb; a salt tonicifier; a surfactant; and an antioxidant.
A50. an EGFR/LGR5 BsAb; a salt tonicifier; a surfactant; and a chelator.
A51. an EGFR/LGR5 BsAb; a salt tonicifier; an amino acid component; and an antioxidant.
A52. an EGFR/LGR5 BsAb; a salt tonicifier; an amino acid component; and a chelator.
A53. an EGFR/LGR5 BsAb; a salt tonicifier; an antioxidant; and a chelator.
A54. an EGFR/LGR5 BsAb; a sugar component; a surfactant; and an amino acid component.
A55. an EGFR/LGR5 BsAb; a sugar component; a surfactant; and an antioxidant.
A56. an EGFR/LGR5 BsAb; a sugar component; a surfactant; and a chelator.
A57. an EGFR/LGR5 BsAb; a sugar component; an amino acid component; and an antioxidant.
A58. an EGFR/LGR5 BsAb; a sugar component; an amino acid component; and a chelator.
A59. an EGFR/LGR5 BsAb; a sugar component; an antioxidant; and a chelator.
A60. an EGFR/LGR5 BsAb; a surfactant; an amino acid component; and an antioxidant.
A61. an EGFR/LGR5 BsAb; a surfactant; an amino acid component; and a chelator.
A62. an EGFR/LGR5 BsAb; a surfactant; an antioxidant; and a chelator.
A63. an EGFR/LGR5 BsAb; an amino acid component; an antioxidant; and a chelator.
A64. an EGFR/LGR5 BsAb; a buffer system; a salt tonicifier; a sugar component; and a surfactant.
A65. an EGFR/LGR5 BsAb; a buffer system; a salt tonicifier; a sugar component; and an amino acid component.
A66. an EGFR/LGR5 BsAb; a buffer system; a salt tonicifier; a sugar component; and an antioxidant.
A67. an EGFR/LGR5 BsAb; a buffer system; a salt tonicifier; a sugar component; and a chelator.
A68. an EGFR/LGR5 BsAb; a buffer system; a salt tonicifier; a surfactant; and an amino acid component.
A69. an EGFR/LGR5 BsAb; a buffer system; a salt tonicifier; a surfactant; and an antioxidant.
A70. an EGFR/LGR5 BsAb; a buffer system; a salt tonicifier; a surfactant; and a chelator.
A71. an EGFR/LGR5 BsAb; a buffer system; a salt tonicifier; an amino acid component; and an antioxidant.
A72. an EGFR/LGR5 BsAb; a buffer system; a salt tonicifier; an amino acid component; and a chelator.
A73. an EGFR/LGR5 BsAb; a buffer system; a salt tonicifier; an antioxidant; and a chelator.
A74. an EGFR/LGR5 BsAb; a buffer system; a sugar component; a surfactant; and an amino acid component.
A75. an EGFR/LGR5 BsAb; a buffer system; a sugar component; a surfactant; and an antioxidant.
A76. an EGFR/LGR5 BsAb; a buffer system; a sugar component; a surfactant; and a chelator.
A77. an EGFR/LGR5 BsAb; a buffer system; a sugar component; an amino acid component; and an antioxidant.
A78. an EGFR/LGR5 BsAb; a buffer system; a sugar component; an amino acid component; and a chelator.
A79. an EGFR/LGR5 BsAb; a buffer system; a sugar component; an antioxidant; and a chelator.
A80. an EGFR/LGR5 BsAb; a buffer system; a surfactant; an amino acid component; and an antioxidant.
A81. an EGFR/LGR5 BsAb; a buffer system; a surfactant; an amino acid component; and a chelator.
A82. an EGFR/LGR5 BsAb; a buffer system; a surfactant; an antioxidant; and a chelator.
A83. an EGFR/LGR5 BsAb; a buffer system; an amino acid component; an antioxidant; and a chelator.
A84. an EGFR/LGR5 BsAb; a salt tonicifier; a sugar component; a surfactant; and an amino acid component.
A85. an EGFR/LGR5 BsAb; a salt tonicifier; a sugar component; a surfactant; and an antioxidant.
A86. an EGFR/LGR5 BsAb; a salt tonicifier; a sugar component; a surfactant; and a chelator.
A87. an EGFR/LGR5 BsAb; a salt tonicifier; a sugar component; an amino acid component; and an antioxidant.
A88. an EGFR/LGR5 BsAb; a salt tonicifier; a sugar component; an amino acid component; and a chelator.
A89. an EGFR/LGR5 BsAb; a salt tonicifier; a sugar component; an antioxidant; and a chelator.
A90. an EGFR/LGR5 BsAb; a salt tonicifier; a surfactant; an amino acid component; and an antioxidant.
A91. an EGFR/LGR5 BsAb; a salt tonicifier; a surfactant; an amino acid component; and a chelator.
A92. an EGFR/LGR5 BsAb; a salt tonicifier; a surfactant; an antioxidant; and a chelator.
A93. an EGFR/LGR5 BsAb; a salt tonicifier; an amino acid component; an antioxidant; and a chelator.
A94. an EGFR/LGR5 BsAb; a sugar component; a surfactant; an amino acid component; and an antioxidant.
A95. an EGFR/LGR5 BsAb; a sugar component; a surfactant; an amino acid component; and a chelator.
A96. an EGFR/LGR5 BsAb; a sugar component; a surfactant; an antioxidant; and a chelator.
A97. an EGFR/LGR5 BsAb; a sugar component; an amino acid component; an antioxidant; and a chelator.
A98. an EGFR/LGR5 BsAb; a surfactant; an amino acid component; an antioxidant; and a chelator.
A99. an EGFR/LGR5 BsAb; a buffer system; a salt tonicifier; a sugar component; a surfactant; and an amino acid component.
A100. an EGFR/LGR5 BsAb; a buffer system; a salt tonicifier; a sugar component; a surfactant; and an antioxidant.
A101. an EGFR/LGR5 BsAb; a buffer system; a salt tonicifier; a sugar component; a surfactant; and a chelator.
A102. an EGFR/LGR5 BsAb; a buffer system; a salt tonicifier; a sugar component; an amino acid component; and an antioxidant.
A103. an EGFR/LGR5 BsAb; a buffer system; a salt tonicifier; a sugar component; an amino acid component; and a chelator.
A104. an EGFR/LGR5 BsAb; a buffer system; a salt tonicifier; a sugar component; an antioxidant; and a chelator.
A105. an EGFR/LGR5 BsAb; a buffer system; a salt tonicifier; a surfactant; an amino acid component; and an antioxidant.
A106. an EGFR/LGR5 BsAb; a buffer system; a salt tonicifier; a surfactant; an amino acid component; and a chelator.
A107. an EGFR/LGR5 BsAb; a buffer system; a salt tonicifier; a surfactant; an antioxidant; and a chelator.
A108. an EGFR/LGR5 BsAb; a buffer system; a salt tonicifier; an amino acid component; an antioxidant; and a chelator.
A109. an EGFR/LGR5 BsAb; a buffer system; a sugar component; a surfactant; an amino acid component; and an antioxidant.
A110. an EGFR/LGR5 BsAb; a buffer system; a sugar component; a surfactant; an amino acid component; and a chelator.
A111. an EGFR/LGR5 BsAb; a buffer system; a sugar component; a surfactant; an antioxidant; and a chelator.
A112. an EGFR/LGR5 BsAb; a buffer system; a sugar component; an amino acid component; an antioxidant; and a chelator.
A113. an EGFR/LGR5 BsAb; a buffer system; a surfactant; an amino acid component; an antioxidant; and a chelator.
A114. an EGFR/LGR5 BsAb; a salt tonicifier; a sugar component; a surfactant; an amino acid component; and an antioxidant.
A115. an EGFR/LGR5 BsAb; a salt tonicifier; a sugar component; a surfactant; an amino acid component; and a chelator.
A116. an EGFR/LGR5 BsAb; a salt tonicifier; a sugar component; a surfactant; an antioxidant; and a chelator.
A117. an EGFR/LGR5 BsAb; a salt tonicifier; a sugar component; an amino acid component; an antioxidant; and a chelator.
A118. an EGFR/LGR5 BsAb; a salt tonicifier; a surfactant; an amino acid component; an antioxidant; and a chelator.
A119. an EGFR/LGR5 BsAb; a sugar component; a surfactant; an amino acid component; an antioxidant; and a chelator.
A120. an EGFR/LGR5 BsAb; a buffer system; a salt tonicifier; a sugar component; a surfactant; an amino acid component; and an antioxidant.
A121. an EGFR/LGR5 BsAb; a buffer system; a salt tonicifier; a sugar component; a surfactant; an amino acid component; and a chelator.
A122. an EGFR/LGR5 BsAb; a buffer system; a salt tonicifier; a sugar component; a surfactant; an antioxidant; and a chelator.
A123. an EGFR/LGR5 BsAb; a buffer system; a salt tonicifier; a sugar component; an amino acid component; an antioxidant; and a chelator.
A124. an EGFR/LGR5 BsAb; a buffer system; a salt tonicifier; a surfactant; an amino acid component; an antioxidant; and a chelator.
A125. an EGFR/LGR5 BsAb; a buffer system; a sugar component; a surfactant; an amino acid component; an antioxidant; and a chelator.
A126. an EGFR/LGR5 BsAb; a salttonicifier; a sugar component; a surfactant; an amino acid component; an antioxidant; and a chelator.
A127. an EGFR/LGR5 BsAb; a buffer system; a salt tonicifier; a sugar component; a surfactant; an amino acid component; an antioxidant; and a chelator.

The bispecific antibody, a functional part, derivative or variant thereof, comprising a first binding domain binding to EGFR and a second binding domain binding to LGR5 denoted as EGFR/LGR5 BsAb in A1-A127, preferably comprises a full length IgG antibody.

B) Embodiments of Combinations of Ingredients for Inclusion with Solution Concentrations

The following numbered paragraphs B1 to B128 disclose specific embodiments of the invention, in which the pharmaceutical composition comprises (or consists of, optionally along with water for injection in the case of aqueous compositions) and is characterised by:
B1. an EGFR/LGR5 BsAb; and a pH of 4-8.5.
B2. an EGFR/LGR5 BsAb; 1-100 mM buffer system; and a pH of 4-8.5.
B3. an EGFR/LGR5 BsAb; a pH of 4-8.5; and 3-200 mM salt tonicifier.
B4. an EGFR/LGR5 BsAb; a pH of 4-8.5; and 20-400 mM sugar component.
B5. an EGFR/LGR5 BsAb; a pH of 4-8.5; and 0.01-2 mg/mL surfactant.
B6. an EGFR/LGR5 BsAb; a pH of 4-8.5; and 5-150 mM amino acid component.
B7. an EGFR/LGR5 BsAb; a pH of 4-8.5; and 0.1-100 mM antioxidant.
B8. an EGFR/LGR5 BsAb; a pH of 4-8.5; and 0.001-0.5 mM chelator.
B9. an EGFR/LGR5 BsAb; 1-100 mM buffer system; a pH of 4-8.5; and 3-200 mM salt tonicifier.
B10. an EGFR/LGR5 BsAb; 1-100 mM buffer system; a pH of 4-8.5; and 20-400 mM sugar component.
B11. an EGFR/LGR5 BsAb; 1-100 mM buffer system; a pH of 4-8.5; and 0.01-2 mg/mL surfactant.
B12. an EGFR/LGR5 BsAb; 1-100 mM buffer system; a pH of 4-8.5; and 5-150 mM amino acid component.
B13. an EGFR/LGR5 BsAb; 1-100 mM buffer system; a pH of 4-8.5; and 0.1-100 mM antioxidant.
B14. an EGFR/LGR5 BsAb; 1-100 mM buffer system; a pH of 4-8.5; and 0.001-0.5 mM chelator.
B15. an EGFR/LGR5 BsAb; a pH of 4-8.5; 3-200 mM salt tonicifier; and 20-400 mM sugar component.
B16. an EGFR/LGR5 BsAb; a pH of 4-8.5; 3-200 mM salt tonicifier; and 0.01-2 mg/mL surfactant.
B17. an EGFR/LGR5 BsAb; a pH of 4-8.5; 3-200 mM salt tonicifier; and 5-150 mM amino acid component.
B18. an EGFR/LGR5 BsAb; a pH of 4-8.5; 3-200 mM salt tonicifier; and 0.1-100 mM antioxidant.
B19. an EGFR/LGR5 BsAb; a pH of 4-8.5; 3-200 mM salt tonicifier; and 0.001-0.5 mM chelator.
B20. an EGFR/LGR5 BsAb; a pH of 4-8.5; 20-400 mM sugar component; and 0.01-2 mg/mL surfactant.
B21. an EGFR/LGR5 BsAb; a pH of 4-8.5; 20-400 mM sugar component; and 5-150 mM amino acid component.
B22. an EGFR/LGR5 BsAb; a pH of 4-8.5; 20-400 mM sugar component; and 0.1-100 mM antioxidant.
B23. an EGFR/LGR5 BsAb; a pH of 4-8.5; 20-400 mM sugar component; and 0.001-0.5 mM chelator.
B24. an EGFR/LGR5 BsAb; a pH of 4-8.5; 0.01-2 mg/mL surfactant; and 5-150 mM amino acid component.
B25. an EGFR/LGR5 BsAb; a pH of 4-8.5; 0.01-2 mg/mL surfactant; and 0.1-100 mM antioxidant.
B26. an EGFR/LGR5 BsAb; a pH of 4-8.5; 0.01-2 mg/mL surfactant; and 0.001-0.5 mM chelator.
B27. an EGFR/LGR5 BsAb; a pH of 4-8.5; 5-150 mM amino acid component; and 0.1-100 mM antioxidant.
B28. an EGFR/LGR5 BsAb; a pH of 4-8.5; 5-150 mM amino acid component; and 0.001-0.5 mM chelator.
B29. an EGFR/LGR5 BsAb; a pH of 4-8.5; 0.1-100 mM antioxidant; and 0.001-0.5 mM chelator.
B30. an EGFR/LGR5 BsAb; 1-100 mM buffer system; a pH of 4-8.5; 3-200 mM salt tonicifier; and 20-400 mM sugar component.
B31. an EGFR/LGR5 BsAb; 1-100 mM buffer system; a pH of 4-8.5; 3-200 mM salt tonicifier; and 0.01-2 mg/mL surfactant.
B32. an EGFR/LGR5 BsAb; 1-100 mM buffer system; a pH of 4-8.5; 3-200 mM salt tonicifier; and 5-150 mM amino acid component.
B33. an EGFR/LGR5 BsAb; 1-100 mM buffer system; a pH of 4-8.5; 3-200 mM salt tonicifier; and 0.1-100 mM antioxidant.
B34. an EGFR/LGR5 BsAb; 1-100 mM buffer system; a pH of 4-8.5; 3-200 mM salt tonicifier; and 0.001-0.5 mM chelator.
B35. an EGFR/LGR5 BsAb; 1-100 mM buffer system; a pH of 4-8.5; 20-400 mM sugar component; and 0.01-2 mg/mL surfactant.
B36. an EGFR/LGR5 BsAb; 1-100 mM buffer system; a pH of 4-8.5; 20-400 mM sugar component; and 5-150 mM amino acid component.
B37. an EGFR/LGR5 BsAb; 1-100 mM buffer system; a pH of 4-8.5; 20-400 mM sugar component; and 0.1-100 mM antioxidant.
B38. an EGFR/LGR5 BsAb; 1-100 mM buffer system; a pH of 4-8.5; 20-400 mM sugar component; and 0.001-0.5 mM chelator.
B39. an EGFR/LGR5 BsAb; 1-100 mM buffer system; a pH of 4-8.5; 0.01-2 mg/mL surfactant; and 5-150 mM amino acid component.
B40. an EGFR/LGR5 BsAb; 1-100 mM buffer system; a pH of 4-8.5; 0.01-2 mg/mL surfactant; and 0.1-100 mM antioxidant.
B41. an EGFR/LGR5 BsAb; 1-100 mM buffer system; a pH of 4-8.5; 0.01-2 mg/mL surfactant; and 0.001-0.5 mM chelator.
B42. an EGFR/LGR5 BsAb; 1-100 mM buffer system; a pH of 4-8.5; 5-150 mM amino acid component; and 0.1-100 mM antioxidant.
B43. an EGFR/LGR5 BsAb; 1-100 mM buffer system; a pH of 4-8.5; 5-150 mM amino acid component; and 0.001-0.5 mM chelator.
B44. an EGFR/LGR5 BsAb; 1-100 mM buffer system; a pH of 4-8.5; 0.1-100 mM antioxidant; and 0.001-0.5 mM chelator.
B45. an EGFR/LGR5 BsAb; a pH of 4-8.5; 3-200 mM salt tonicifier; 20-400 mM sugar component; and 0.01-2 mg/mL surfactant.
B46. an EGFR/LGR5 BsAb; a pH of 4-8.5; 3-200 mM salt tonicifier; 20-400 mM sugar component; and 5-150 mM amino acid component.
B47. an EGFR/LGR5 BsAb; a pH of 4-8.5; 3-200 mM salt tonicifier; 20-400 mM sugar component; and 0.1-100 mM antioxidant.
B48. an EGFR/LGR5 BsAb; a pH of 4-8.5; 3-200 mM salt tonicifier; 20-400 mM sugar component; and 0.001-0.5 mM chelator.
B49. an EGFR/LGR5 BsAb; a pH of 4-8.5; 3-200 mM salt tonicifier; 0.01-2 mg/mL surfactant; and 5-150 mM amino acid component.
B50. an EGFR/LGR5 BsAb; a pH of 4-8.5; 3-200 mM salt tonicifier; 0.01-2 mg/mL surfactant; and 0.1-100 mM antioxidant.
B51. an EGFR/LGR5 BsAb; a pH of 4-8.5; 3-200 mM salt tonicifier; 0.01-2 mg/mL surfactant; and 0.001-0.5 mM chelator.
B52. an EGFR/LGR5 BsAb; a pH of 4-8.5; 3-200 mM salt tonicifier; 5-150 mM amino acid component; and 0.1-100 mM antioxidant.
B53. an EGFR/LGR5 BsAb; a pH of 4-8.5; 3-200 mM salt tonicifier; 5-150 mM amino acid component; and 0.001-0.5 mM chelator.
B54. an EGFR/LGR5 BsAb; a pH of 4-8.5; 3-200 mM salt tonicifier; 0.1-100 mM antioxidant; and 0.001-0.5 mM chelator.
B55. an EGFR/LGR5 BsAb; a pH of 4-8.5; 20-400 mM sugar component; 0.01-2 mg/mL surfactant; and 5-150 mM amino acid component.
B56. an EGFR/LGR5 BsAb; a pH of 4-8.5; 20-400 mM sugar component; 0.01-2 mg/mL surfactant; and 0.1-100 mM antioxidant.
B57. an EGFR/LGR5 BsAb; a pH of 4-8.5; 20-400 mM sugar component; 0.01-2 mg/mL surfactant; and 0.001-0.5 mM chelator.
B58. an EGFR/LGR5 BsAb; a pH of 4-8.5; 20-400 mM sugar component; 5-150 mM amino acid component; and 0.1-100 mM antioxidant.
B59. an EGFR/LGR5 BsAb; a pH of 4-8.5; 20-400 mM sugar component; 5-150 mM amino acid component; and 0.001-0.5 mM chelator.
B60. an EGFR/LGR5 BsAb; a pH of 4-8.5; 20-400 mM sugar component; 0.1-100 mM antioxidant; and 0.001-0.5 mM chelator.
B61. an EGFR/LGR5 BsAb; a pH of 4-8.5; 0.01-2 mg/mL surfactant; 5-150 mM amino acid component; and 0.1-100 mM antioxidant.
B62. an EGFR/LGR5 BsAb; a pH of 4-8.5; 0.01-2 mg/mL surfactant; 5-150 mM amino acid component; and 0.001-0.5 mM chelator.
B63. an EGFR/LGR5 BsAb; a pH of 4-8.5; 0.01-2 mg/mL surfactant; 0.1-100 mM antioxidant; and 0.001-0.5 mM chelator.
B64. an EGFR/LGR5 BsAb; a pH of 4-8.5; 5-150 mM amino acid component; 0.1-100 mM antioxidant; and 0.001-0.5 mM chelator.
B65. an EGFR/LGR5 BsAb; 1-100 mM buffer system; a pH of 4-8.5; 3-200 mM salt tonicifier; 20-400 mM sugar component; and 0.01-2 mg/mL surfactant.
B66. an EGFR/LGR5 BsAb; 1-100 mM buffer system; a pH of 4-8.5; 3-200 mM salt tonicifier; 20-400 mM sugar component; and 5-150 mM amino acid component.
B67. an EGFR/LGR5 BsAb; 1-100 mM buffer system; a pH of 4-8.5; 3-200 mM salt tonicifier; 20-400 mM sugar component; and 0.1-100 mM antioxidant.
B68. an EGFR/LGR5 BsAb; 1-100 mM buffer system; a pH of 4-8.5; 3-200 mM salt tonicifier; 20-400 mM sugar component; and 0.001-0.5 mM chelator.
B69. an EGFR/LGR5 BsAb; 1-100 mM buffer system; a pH of 4-8.5; 3-200 mM salt tonicifier; 0.01-2 mg/mL surfactant; and 5-150 mM amino acid component.
B70. an EGFR/LGR5 BsAb; 1-100 mM buffer system; a pH of 4-8.5; 3-200 mM salt tonicifier; 0.01-2 mg/mL surfactant; and 0.1-100 mM antioxidant.
B71. an EGFR/LGR5 BsAb; 1-100 mM buffer system; a pH of 4-8.5; 3-200 mM salt tonicifier; 0.01-2 mg/mL surfactant; and 0.001-0.5 mM chelator.
B72. an EGFR/LGR5 BsAb; 1-100 mM buffer system; a pH of 4-8.5; 3-200 mM salt tonicifier; 5-150 mM amino acid component; and 0.1-100 mM antioxidant.
B73. an EGFR/LGR5 BsAb; 1-100 mM buffer system; a pH of 4-8.5; 3-200 mM salt tonicifier; 5-150 mM amino acid component; and 0.001-0.5 mM chelator.
B74. an EGFR/LGR5 BsAb; 1-100 mM buffer system; a pH of 4-8.5; 3-200 mM salt tonicifier; 0.1-100 mM antioxidant; and 0.001-0.5 mM chelator.
B75. an EGFR/LGR5 BsAb; 1-100 mM buffer system; a pH of 4-8.5; 20-400 mM sugar component; 0.01-2 mg/mL surfactant; and 5-150 mM amino acid component.
B76. an EGFR/LGR5 BsAb; 1-100 mM buffer system; a pH of 4-8.5; 20-400 mM sugar component; 0.01-2 mg/mL surfactant; and 0.1-100 mM antioxidant.
B77. an EGFR/LGR5 BsAb; 1-100 mM buffer system; a pH of 4-8.5; 20-400 mM sugar component; 0.01-2 mg/mL surfactant; and 0.001-0.5 mM chelator.
B78. an EGFR/LGR5 BsAb; 1-100 mM buffer system; a pH of 4-8.5; 20-400 mM sugar component; 5-150 mM amino acid component; and 0.1-100 mM antioxidant.
B79. an EGFR/LGR5 BsAb; 1-100 mM buffer system; a pH of 4-8.5; 20-400 mM sugar component; 5-150 mM amino acid component; and 0.001-0.5 mM chelator.
B80. an EGFR/LGR5 BsAb; 1-100 mM buffer system; a pH of 4-8.5; 20-400 mM sugar component; 0.1-100 mM antioxidant; and 0.001-0.5 mM chelator.
B81. an EGFR/LGR5 BsAb; 1-100 mM buffer system; a pH of 4-8.5; 0.01-2 mg/mL surfactant; 5-150 mM amino acid component; and 0.1-100 mM antioxidant.
B82. an EGFR/LGR5 BsAb; 1-100 mM buffer system; a pH of 4-8.5; 0.01-2 mg/mL surfactant; 5-150 mM amino acid component; and 0.001-0.5 mM chelator.
B83. an EGFR/LGR5 BsAb; 1-100 mM buffer system; a pH of 4-8.5; 0.01-2 mg/mL surfactant; 0.1-100 mM antioxidant; and 0.001-0.5 mM chelator.
B84. an EGFR/LGR5 BsAb; 1-100 mM buffer system; a pH of 4-8.5; 5-150 mM amino acid component; 0.1-100 mM antioxidant; and 0.001-0.5 mM chelator.
B85. an EGFR/LGR5 BsAb; a pH of 4-8.5; 3-200 mM salt tonicifier; 20-400 mM sugar component; 0.01-2 mg/mL surfactant; and 5-150 mM amino acid component.
B86. an EGFR/LGR5 BsAb; a pH of 4-8.5; 3-200 mM salt tonicifier; 20-400 mM sugar component; 0.01-2 mg/mL surfactant; and 0.1-100 mM antioxidant.
B87. an EGFR/LGR5 BsAb; a pH of 4-8.5; 3-200 mM salt tonicifier; 20-400 mM sugar component; 0.01-2 mg/mL surfactant; and 0.001-0.5 mM chelator.
B88. an EGFR/LGR5 BsAb; a pH of 4-8.5; 3-200 mM salt tonicifier; 20-400 mM sugar component; 5-150 mM amino acid component; and 0.1-100 mM antioxidant.
B89. an EGFR/LGR5 BsAb; a pH of 4-8.5; 3-200 mM salt tonicifier; 20-400 mM sugar component; 5-150 mM amino acid component; and 0.001-0.5 mM chelator.
B90. an EGFR/LGR5 BsAb; a pH of 4-8.5; 3-200 mM salt tonicifier; 20-400 mM sugar component; 0.1-100 mM antioxidant; and 0.001-0.5 mM chelator.
B91. an EGFR/LGR5 BsAb; a pH of 4-8.5; 3-200 mM salt tonicifier; 0.01-2 mg/mL surfactant; 5-150 mM amino acid component; and 0.1-100 mM antioxidant.
B92. an EGFR/LGR5 BsAb; a pH of 4-8.5; 3-200 mM salt tonicifier; 0.01-2 mg/mL surfactant; 5-150 mM amino acid component; and 0.001-0.5 mM chelator.
B93. an EGFR/LGR5 BsAb; a pH of 4-8.5; 3-200 mM salt tonicifier; 0.01-2 mg/mL surfactant; 0.1-100 mM antioxidant; and 0.001-0.5 mM chelator.
B94. an EGFR/LGR5 BsAb; a pH of 4-8.5; 3-200 mM salt tonicifier; 5-150 mM amino acid component; 0.1-100 mM antioxidant; and 0.001-0.5 mM chelator.
B95. an EGFR/LGR5 BsAb; a pH of 4-8.5; 20-400 mM sugar component; 0.01-2 mg/mL surfactant; 5-150 mM amino acid component; and 0.1-100 mM antioxidant.
B96. an EGFR/LGR5 BsAb; a pH of 4-8.5; 20-400 mM sugar component; 0.01-2 mg/mL surfactant; 5-150 mM amino acid component; and 0.001-0.5 mM chelator.
B97. an EGFR/LGR5 BsAb; a pH of 4-8.5; 20-400 mM sugar component; 0.01-2 mg/mL surfactant; 0.1-100 mM antioxidant; and 0.001-0.5 mM chelator.
B98. an EGFR/LGR5 BsAb; a pH of 4-8.5; 20-400 mM sugar component; 5-150 mM amino acid component; 0.1-100 mM antioxidant; and 0.001-0.5 mM chelator.
B99. an EGFR/LGR5 BsAb; a pH of 4-8.5; 0.01-2 mg/mL surfactant; 5-150 mM amino acid component; 0.1-100 mM antioxidant; and 0.001-0.5 mM chelator.
B100. an EGFR/LGR5 BsAb; 1-100 mM buffer system; a pH of 4-8.5; 3-200 mM salt tonicifier; 20-400 mM sugar component; 0.01-2 mg/mL surfactant; and 5-150 mM amino acid component.
B101. an EGFR/LGR5 BsAb; 1-100 mM buffer system; a pH of 4-8.5; 3-200 mM salt tonicifier; 20-400 mM sugar component; 0.01-2 mg/mL surfactant; and 0.1-100 mM antioxidant.
B102. an EGFR/LGR5 BsAb; 1-100 mM buffer system; a pH of 4-8.5; 3-200 mM salt tonicifier; 20-400 mM sugar component; 0.01-2 mg/mL surfactant; and 0.001-0.5 mM chelator.
B103. an EGFR/LGR5 BsAb; 1-100 mM buffer system; a pH of 4-8.5; 3-200 mM salt tonicifier; 20-400 mM sugar component; 5-150 mM amino acid component; and 0.1-100 mM antioxidant.
B104. an EGFR/LGR5 BsAb; 1-100 mM buffer system; a pH of 4-8.5; 3-200 mM salt tonicifier; 20-400 mM sugar component; 5-150 mM amino acid component; and 0.001-0.5 mM chelator.
B105. an EGFR/LGR5 BsAb; 1-100 mM buffer system; a pH of 4-8.5; 3-200 mM salt tonicifier; 20-400 mM sugar component; 0.1-100 mM antioxidant; and 0.001-0.5 mM chelator.
B106. an EGFR/LGR5 BsAb; 1-100 mM buffer system; a pH of 4-8.5; 3-200 mM salt tonicifier; 0.01-2 mg/mL surfactant; 5-150 mM amino acid component; and 0.1-100 mM antioxidant.
B107. an EGFR/LGR5 BsAb; 1-100 mM buffer system; a pH of 4-8.5; 3-200 mM salt tonicifier; 0.01-2 mg/mL surfactant; 5-150 mM amino acid component; and 0.001-0.5 mM chelator.
B108. an EGFR/LGR5 BsAb; 1-100 mM buffer system; a pH of 4-8.5; 3-200 mM salt tonicifier; 0.01-2 mg/mL surfactant; 0.1-100 mM antioxidant; and 0.001-0.5 mM chelator.
B109. an EGFR/LGR5 BsAb; 1-100 mM buffer system; a pH of 4-8.5; 3-200 mM salt tonicifier; 5-150 mM amino acid component; 0.1-100 mM antioxidant; and 0.001-0.5 mM chelator.
B110. an EGFR/LGR5 BsAb; 1-100 mM buffer system; a pH of 4-8.5; 20-400 mM sugar component; 0.01-2 mg/mL surfactant; 5-150 mM amino acid component; and 0.1-100 mM antioxidant.
B111. an EGFR/LGR5 BsAb; 1-100 mM buffer system; a pH of 4-8.5; 20-400 mM sugar component; 0.01-2 mg/mL surfactant; 5-150 mM amino acid component; and 0.001-0.5 mM chelator.
B112. an EGFR/LGR5 BsAb; 1-100 mM buffer system; a pH of 4-8.5; 20-400 mM sugar component; 0.01-2 mg/mL surfactant; 0.1-100 mM antioxidant; and 0.001-0.5 mM chelator.
B113. an EGFR/LGR5 BsAb; 1-100 mM buffer system; a pH of 4-8.5; 20-400 mM sugar component; 5-150 mM amino acid component; 0.1-100 mM antioxidant; and 0.001-0.5 mM chelator.
B114. an EGFR/LGR5 BsAb; 1-100 mM buffer system; a pH of 4-8.5; 0.01-2 mg/mL surfactant; 5-150 mM amino acid component; 0.1-100 mM antioxidant; and 0.001-0.5 mM chelator.
B115. an EGFR/LGR5 BsAb; a pH of 4-8.5; 3-200 mM salt tonicifier; 20-400 mM sugar component; 0.01-2 mg/mL surfactant; 5-150 mM amino acid component; and 0.1-100 mM antioxidant.
B116. an EGFR/LGR5 BsAb; a pH of 4-8.5; 3-200 mM salt tonicifier; 20-400 mM sugar component; 0.01-2 mg/mL surfactant; 5-150 mM amino acid component; and 0.001-0.5 mM chelator.
B117. an EGFR/LGR5 BsAb; a pH of 4-8.5; 3-200 mM salt tonicifier; 20-400 mM sugar component; 0.01-2 mg/mL surfactant; 0.1-100 mM antioxidant; and 0.001-0.5 mM chelator.
B118. an EGFR/LGR5 BsAb; a pH of 4-8.5; 3-200 mM salt tonicifier; 20-400 mM sugar component; 5-150 mM amino acid component; 0.1-100 mM antioxidant; and 0.001-0.5 mM chelator.
B119. an EGFR/LGR5 BsAb; a pH of 4-8.5; 3-200 mM salt tonicifier; 0.01-2 mg/mL surfactant; 5-150 mM amino acid component; 0.1-100 mM antioxidant; and 0.001-0.5 mM chelator.
B120. an EGFR/LGR5 BsAb; a pH of 4-8.5; 20-400 mM sugar component; 0.01-2 mg/mL surfactant; 5-150 mM amino acid component; 0.1-100 mM antioxidant; and 0.001-0.5 mM chelator.
B121. an EGFR/LGR5 BsAb; 1-100 mM buffer system; a pH of 4-8.5; 3-200 mM salt tonicifier; 20-400 mM sugar component; 0.01-2 mg/mL surfactant; 5-150 mM amino acid component; and 0.1-100 mM antioxidant.
B122. an EGFR/LGR5 BsAb; 1-100 mM buffer system; a pH of 4-8.5; 3-200 mM salt tonicifier; 20-400 mM sugar component; 0.01-2 mg/mL surfactant; 5-150 mM amino acid component; and 0.001-0.5 mM chelator.
B123. an EGFR/LGR5 BsAb; 1-100 mM buffer system; a pH of 4-8.5; 3-200 mM salt tonicifier; 20-400 mM sugar component; 0.01-2 mg/mLsurfactant; 0.1-100 mM antioxidant; and 0.001-0.5 mM chelator.
B124. an EGFR/LGR5 BsAb; 1-100 mM buffer system; a pH of 4-8.5; 3-200 mM salt tonicifier; 20-400 mM sugar component; 5-150 mM amino acid component; 0.1-100 mM antioxidant; and 0.001-0.5 mM chelator.
B125. an EGFR/LGR5 BsAb; 1-100 mM buffer system; a pH of 4-8.5; 3-200 mM salt tonicifier; 0.01-2 mg/mL surfactant; 5-150 mM amino acid component; 0.1-100 mM antioxidant; and 0.001-0.5 mM chelator.
B126. an EGFR/LGR5 BsAb; 1-100 mM buffer system; a pH of 4-8.5; 20-400 mM sugar component; 0.01-2 mg/mL surfactant; 5-150 mM amino acid component; 0.1-100 mM antioxidant; and 0.001-0.5 mM chelator.
B127. an EGFR/LGR5 BsAb; a pH of 4-8.5; 3-200 mM salt tonicifier; 20-400 mM sugar component; 0.01-2 mg/mL surfactant; 5-150 mM amino acid component; 0.1-100 mM antioxidant; and 0.001-0.5 mM chelator.
B128. an EGFR/LGR5 BsAb; 1-100 mM buffer system; a pH of 4-8.5; 3-200 mM salt tonicifier; 20-400 mM sugar component; 0.01-2 mg/mL surfactant; 5-150 mM amino acid component; 0.1-100 mM antioxidant; and 0.001-0.5 mM chelator.

The bispecific antibody, a functional part, derivative or variant thereof, comprising a first binding domain binding to EGFR and a second binding domain binding to LGR5 denoted as EGFR/LGR5 BsAb in B1-B128, preferably comprises a full length IgG antibody.

C) Embodiments of Combinations of Ingredients for Inclusion with Molar Ratios

The following numbered paragraphs C1 to C128 disclose specific embodiments of the invention, in which the pharmaceutical composition comprises (or consists of, optionally along with water for injection in the case of aqueous compositions) and is characterised by:
C1. An EGFR/LGR5 BsAb; and a pH of 4-8.5.
C2. An EGFR/LGR5 BsAb; a buffer system in a molar ratio of buffer system to antibody of from 4:1 to 1050:1; and a pH of 4-8.5.
C3. An EGFR/LGR5 BsAb; a pH of 4-8.5; and a salt tonicifier in a molar ratio of salt tonicifier to antibody of from 7:1 to 3000:1.
C4. An EGFR/LGR5 BsAb; a pH of 4-8.5; and a sugar component in a molar ratio of sugar component to antibody of from 100:1 to 5500:1.
C5. An EGFR/LGR5 BsAb; a pH of 4-8.5; and a surfactant in a molar ratio of surfactant to antibody of from 1:11 to 17:1.
C6. An EGFR/LGR5 BsAb; a pH of 4-8.5; and an amino acid component in a molar ratio of amino acid component to antibody of from 10:1 to 2500:1.
C7. An EGFR/LGR5 BsAb; a pH of 4-8.5; and an antioxidant in a molar ratio of antioxidant to antibody of from 2:1 to 750:1.
C8. An EGFR/LGR5 BsAb; a pH of 4-8.5; and a chelator in a molar ratio of chelator to antibody of from 1:420 to 8:1.
C9. An EGFR/LGR5 BsAb; a buffer system in a molar ratio of buffer system to antibody of from 4:1 to 1050:1; a pH of 4-8.5; and a salt tonicifier in a molar ratio of salt tonicifier to antibody of from 7:1 to 3000:1.
C10. An EGFR/LGR5 BsAb; a buffer system in a molar ratio of buffer system to antibody of from 4:1 to 1050:1; a pH of 4-8.5; and a sugar component in a molar ratio of sugar component to antibody of from 100:1 to 5500:1.
C11. An EGFR/LGR5 BsAb; a buffer system in a molar ratio of buffer system to antibody of from 4:1 to 1050:1; a pH of 4-8.5; and a surfactant in a molar ratio of surfactant to antibody of from 1:11 to 17:1.
C12. An EGFR/LGR5 BsAb; a buffer system in a molar ratio of buffer system to antibody of from 4:1 to 1050:1; a pH of 4-8.5; and an amino acid component in a molar ratio of amino acid component to antibody of from 10:1 to 2500:1.
C13. An EGFR/LGR5 BsAb; a buffer system in a molar ratio of buffer system to antibody of from 4:1 to 1050:1; a pH of 4-8.5; and an antioxidant in a molar ratio of antioxidant to antibody of from 2:1 to 750:1.
C14. An EGFR/LGR5 BsAb; a buffer system in a molar ratio of buffer system to antibody of from 4:1 to 1050:1; a pH of 4-8.5; and a chelator in a molar ratio of chelator to antibody of from 1:420 to 8:1.
C15. An EGFR/LGR5 BsAb; a pH of 4-8.5; a salt tonicifier in a molar ratio of salt tonicifier to antibody of from 7:1 to 3000:1; and a sugar component in a molar ratio of sugar component to antibody of from 100:1 to 5500:1.
C16. An EGFR/LGR5 BsAb; a pH of 4-8.5; a salt tonicifier in a molar ratio of salt tonicifier to antibody of from 7:1 to 3000:1; and a surfactant in a molar ratio of surfactant to antibody of from 1:11 to 17:1.
C17. An EGFR/LGR5 BsAb; a pH of 4-8.5; a salt tonicifier in a molar ratio of salt tonicifier to antibody of from 7:1 to 3000:1; and an amino acid component in a molar ratio of amino acid component to antibody of from 10:1 to 2500:1.
C18. An EGFR/LGR5 BsAb; a pH of 4-8.5; a salt tonicifier in a molar ratio of salt tonicifier to antibody of from 7:1 to 3000:1; and an antioxidant in a molar ratio of antioxidant to antibody of from 2:1 to 750:1.
C19. An EGFR/LGR5 BsAb; a pH of 4-8.5; a salt tonicifier in a molar ratio of salt tonicifier to antibody of from 7:1 to 3000:1; and a chelator in a molar ratio of chelator to antibody of from 1:420 to 8:1.
C20. An EGFR/LGR5 BsAb; a pH of 4-8.5; a sugar component in a molar ratio of sugar component to antibody of from 100:1 to 5500:1; and a surfactant in a molar ratio of surfactant to antibody of from 1:11 to 17:1.
C21. An EGFR/LGR5 BsAb; a pH of 4-8.5; a sugar component in a molar ratio of sugar component to antibody of from 100:1 to 5500:1; and an amino acid component in a molar ratio of amino acid component to antibody of from 10:1 to 2500:1.
C22. An EGFR/LGR5 BsAb; a pH of 4-8.5; a sugar component in a molar ratio of sugar component to antibody of from 100:1 to 5500:1; and an antioxidant in a molar ratio of antioxidant to antibody of from 2:1 to 750:1.
C23. An EGFR/LGR5 BsAb; a pH of 4-8.5; a sugar component in a molar ratio of sugar component to antibody of from 100:1 to 5500:1; and a chelator in a molar ratio of chelator to antibody of from 1:420 to 8:1.
C24. An EGFR/LGR5 BsAb; a pH of 4-8.5; a surfactant in a molar ratio of surfactant to antibody of from 1:11 to 17:1; and an amino acid component in a molar ratio of amino acid component to antibody of from 10:1 to 2500:1.
C25. An EGFR/LGR5 BsAb; a pH of 4-8.5; a surfactant in a molar ratio of surfactant to antibody of from 1:11 to 17:1; and an antioxidant in a molar ratio of antioxidant to antibody of from 2:1 to 750:1.
C26. An EGFR/LGR5 BsAb; a pH of 4-8.5; a surfactant in a molar ratio of surfactant to antibody of from 1:11 to 17:1; and a chelator in a molar ratio of chelator to antibody of from 1:420 to 8:1.
C27. An EGFR/LGR5 BsAb; a pH of 4-8.5; an amino acid component in a molar ratio of amino acid component to antibody of from 10:1 to 2500:1; and an antioxidant in a molar ratio of antioxidant to antibody of from 2:1 to 750:1.
C28. An EGFR/LGR5 BsAb; a pH of 4-8.5; an amino acid component in a molar ratio of amino acid component to antibody of from 10:1 to 2500:1; and a chelator in a molar ratio of chelator to antibody of from 1:420 to 8:1.
C29. An EGFR/LGR5 BsAb; a pH of 4-8.5; an antioxidant in a molar ratio of antioxidant to antibody of from 2:1 to 750:1; and a chelator in a molar ratio of chelator to antibody of from 1:420 to 8:1.
C30. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; and a sugar component in a molar ratio of sugar component to antibody of from 100:1 to 5500:1.
C31. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; and a surfactant in a molar ratio of surfactant to antibody of from 1:11 to 17:1.
C32. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; and an amino acid component in a molar ratio of amino acid component to antibody of from 10:1 to 2500:1.
C33. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; and an antioxidant in a molar ratio of antioxidant to antibody of from 2:1 to 750:1.
C34. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; and a chelator in a molar ratio of chelator to antibody of from 1:420 to 8:1.
C35. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; and a surfactant in a molar ratio of surfactant to antibody of from 1:11 to 17:1.
C36. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; and an amino acid component in a molar ratio of amino acid component to antibody of from 10:1 to 2500:1.
C37. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; and an antioxidant in a molar ratio of antioxidant to antibody of from 2:1 to 750:1.
C38. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; and a chelator in a molar ratio of chelator to antibody of from 1:420 to 8:1.
C39. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; and an amino acid component in a molar ratio of amino acid component to antibody of from 10:1 to 2500:1.
C40. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; and an antioxidant in a molar ratio of antioxidant to antibody of from 2:1 to 750:1.
C41. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; and a chelator in a molar ratio of chelator to antibody of from 1:420 to 8:1.
C42. An EGFR/LGR5 BsAb; a buffer system in a molar ratio of buffer system to antibody of from 4:1 to 1050:1; a pH of 4-8.5; an amino acid component in a molar ratio of amino acid component to antibody of from 10:1 to 2500:1; and an antioxidant in a molar ratio of antioxidant to antibody of from 2:1 to 750:1.
C43. An EGFR/LGR5 BsAb; a buffer system in a molar ratio of buffer system to antibody of from 4:1 to 1050:1; a pH of 4-8.5; an amino acid component in a molar ratio of amino acid component to antibody of from 10:1 to 2500:1; and a chelator in a molar ratio of chelator to antibody of from 1:420 to 8:1.
C44. An EGFR/LGR5 BsAb; a buffer system in a molar ratio of buffer system to antibody of from 4:1 to 1050:1; a pH of 4-8.5; an antioxidant in a molar ratio of antioxidant to antibody of from 2:1 to 750:1; and a chelator in a molar ratio of chelator to antibody of from 1:420 to 8:1.
C45. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; and a surfactant in a molar ratio of surfactant to antibody of from 1:11 to 17:1.
C46. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; and an amino acid component in a molar ratio of amino acid component to antibody of from 10:1 to 2500:1.
C47. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; and an antioxidant in a molar ratio of antioxidant to antibody of from 2:1 to 750:1.
C48. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; and a chelator in a molar ratio of chelator to antibody of from 1:420 to 8:1.
C49. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; and an amino acid component in a molar ratio of amino acid component to antibody of from 10:1 to 2500:1.
C50. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; and an antioxidant in a molar ratio of antioxidant to antibody of from 2:1 to 750:1.
C51. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; and a chelator in a molar ratio of chelator to antibody of from 1:420 to 8:1.
C52. An EGFR/LGR5 BsAb; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; and an antioxidant in a molar ratio of antioxidant to antibody of from 2:1 to 750:1.
C53. An EGFR/LGR5 BsAb; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; and a chelator in a molar ratio of chelator to antibody of from 1:420 to 8:1.
C54. An EGFR/LGR5 BsAb; a pH of 4-8.5; a salt tonicifier in a molar ratio of salt tonicifier to antibody of from 7:1 to 3000:1; an antioxidant in a molar ratio of antioxidant to antibody of from 2:1 to 750:1; and a chelator in a molar ratio of chelator to antibody of from 1:420 to 8:1.
C55. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; and an amino acid component in a molar ratio of amino acid component to antibody of from 10:1 to 2500:1.
C56. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; and an antioxidant in a molar ratio of antioxidant to antibody of from 2:1 to 750:1.
C57. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; and a chelator in a molar ratio of chelator to antibody of from 1:420 to 8:1.
C58. An EGFR/LGR5 BsAb; a pH of 4-8.5; a sugar component in a molar ratio of sugar component to antibody of from 100:1 to 5500:1; an amino acid component in a molar ratio of amino acid component to antibody of from 10:1 to 2500:1; and an antioxidant in a molar ratio of antioxidant to antibody of from 2:1 to 750:1.
C59. An EGFR/LGR5 BsAb; a pH of 4-8.5; a sugar component in a molar ratio of sugar component to antibody of from 100:1 to 5500:1; an amino acid component in a molar ratio of amino acid component to antibody of from 10:1 to 2500:1; and a chelator in a molar ratio of chelator to antibody of from 1:420 to 8:1.
C60. An EGFR/LGR5 BsAb; a pH of 4-8.5; a sugar component in a molar ratio of sugar component to antibody of from 100:1 to 5500:1; an antioxidant in a molar ratio of antioxidant to antibody of from 2:1 to 750:1; and a chelator in a molar ratio of chelator to antibody of from 1:420 to 8:1.
C61. An EGFR/LGR5 BsAb; a pH of 4-8.5; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C62. An EGFR/LGR5 BsAb; a pH of 4-8.5; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C63. An EGFR/LGR5 BsAb; a pH of 4-8.5; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C64. An EGFR/LGR5 BsAb; a pH of 4-8.5; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C65. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; and a surfactant in a molar ratio of surfactant to antibody of from 1:11 to 17:1.
C66. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; and an amino acid component in a molar ratio of amino acid component to antibody of from 10:1 to 2500:1.
C67. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; and an antioxidant in a molar ratio of antioxidant to antibody of from 2:1 to 750:1.
C68. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; and a chelator in a molar ratio of chelator to antibody of from 1:420 to 8:1.
C69. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; and an amino acid component in a molar ratio of amino acid component to antibody of from 10:1 to 2500:1.
C70. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; and an antioxidant in a molar ratio of antioxidant to antibody of from 2:1 to 750:1.
C71. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; and a chelator in a molar ratio of chelator to antibody of from 1:420 to 8:1.
C72. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; and an antioxidant in a molar ratio of antioxidant to antibody of from 2:1 to 750:1.
C73. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; and a chelator in a molar ratio of chelator to antibody of from 1:420 to 8:1.
C74. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; an antioxidant in a molar ratio of antioxidant to antibody of from 2:1 to 750:1; and a chelator in a molar ratio of chelator to antibody of from 1:420 to 8:1.
C75. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; and an amino acid component in a molar ratio of amino acid component to antibody of from 10:1 to 2500:1.
C76. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; and an antioxidant in a molar ratio of antioxidant to antibody of from 2:1 to 750:1.
C77. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; and a chelator in a molar ratio of chelatorto antibody of from 1:420 to 8:1.
C78. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; an amino acid component in a molar ratio of amino acid component to antibody of from 10:1 to 2500:1; and an antioxidant in a molar ratio of antioxidant to antibody of from 2:1 to 750:1.
C79. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; an amino acid component in a molar ratio of amino acid component to antibody of from 10:1 to 2500:1; and a chelator in a molar ratio of chelator to antibody of from 1:420 to 8:1.
C80. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; an antioxidant in a molar ratio of antioxidant to antibody of from 2:1 to 750:1; and a chelator in a molar ratio of chelatorto antibody of from 1:420 to 8:1.
C81. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C82. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C83. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C84. An EGFR/LGR5 BsAb; a buffer system in a molar ratio of buffer system to antibody of from 4:1 to 1050:1; a pH of 4-8.5; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C85. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and an amino acid component in a molar ratio of amino acid component to antibody of from 10:1 to 2500:1.
C86. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and an antioxidant in a molar ratio of antioxidant to antibody of from 2:1 to 750:1.
C87. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and a chelator in a molar ratio of chelator to antibody of from 1:420 to 8:1.
C88. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; and an antioxidant in a molar ratio of antioxidant to antibody of from 2:1 to 750:1.
C89. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; and a chelator in a molar ratio of chelator to antibody of from 1:420 to 8:1.
C90. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; an antioxidant in a molar ratio of antioxidant to antibody of from 2:1 to 750:1; and a chelator in a molar ratio of chelatorto antibody of from 1:420 to 8:1.
C91. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C92. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C93. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C94. An EGFR/LGR5 BsAb; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C95. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C96. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C97. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C98. An EGFR/LGR5 BsAb; a pH of 4-8.5; a sugar component in a molar ratio of sugar component to antibody of from 100:1 to 5500:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C99. An EGFR/LGR5 BsAb; a pH of 4-8.5; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C100. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and an amino acid component in a molar ratio of amino acid component to antibody of from 10:1 to 2500:1.
C101. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and an antioxidant in a molar ratio of antioxidant to antibody of from 2:1 to 750:1.
C102. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; and a chelator in a molar ratio of chelator to antibody of from 1:420 to 8:1.
C103. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; and an antioxidant in a molar ratio of antioxidant to antibody of from 2:1 to 750:1.
C104. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; and a chelator in a molar ratio of chelator to antibody of from 1:420 to 8:1.
C105. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; an antioxidant in a molar ratio of antioxidant to antibody of from 2:1 to 750:1; and a chelator in a molar ratio of chelator to antibody of from 1:420 to 8:1.
C106. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C107. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C108. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C109. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C110. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C111. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C112. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C113. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C114. An EGFR/LGR5 BsAb; a buffer system in a molar ratio of buffer system to antibody of from 4:1 to 1050:1; a pH of 4-8.5; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C115. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C116. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and a chelator in a molar ratio of chelatorto antibody of from 1:420 to 8:1.
C117. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C118. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C119. An EGFR/LGR5 BsAb; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C120. An EGFR/LGR5 BsAb; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C121. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and an antioxidant in a molar ratio of antioxidant to antibody of from 2:1 to 750:1.
C122. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; and a chelator in a molar ratio of chelator to antibody of from 1:420 to 8:1.
C123. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an antioxidant in a molar ratio of antioxidant to antibody of from 2:1 to 750:1; and a chelator in a molar ratio of chelator to antibody of from 1:420 to 8:1.
C124. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelatorto antibody of from 1:420 to 8:1.
C125. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C126. An EGFR/LGR5 BsAb; a buffer system in a molar ratio of buffer system to antibody of from 4:1 to 1050:1; a pH of 4-8.5; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C127. An EGFR/LGR5 BsAb; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.
C128. An EGFR/LGR5 BsAb; a buffer system in a molar ratio of buffer system to antibody of from 4:1 to 1050:1; a pH of 4-8.5; a salt tonicifier in a molar ratio of salt tonicifier to antibody of from 7:1 to 3000:1; a sugar component in a molar ratio of sugar component to antibody of from 100:1 to 5500:1; a surfactant in a molar ratio of surfactant to antibody of from 1:11 to 17:1; an amino acid component in a molar ratio of amino acid component to antibody of from 10:1 to 2500:1; an antioxidant in a molar ratio of antioxidant to antibody of from 2:1 to 750:1; and a chelator in a molar ratio of chelator to antibody of from 1:420 to 8:1.

The bispecific antibody, a functional part, derivative or variant thereof, comprising a first binding domain binding to EGFR and a second binding domain binding to LGR5 denoted as EGFR/LGR5 BsAb in C1-C128, preferably comprises a full length IgG antibody.

### Bispecific Antibody

The pharmaceutical composition comprises a multispecific or preferably a bispecific antibody, functional part or derivative thereof, comprising a first binding domain binding to EGFR and a second binding domain binding to LGR5, preferably a bispecific antibody (BsAb), having both anti-EGFR and anti-LGR5 binding properties (EGFR/LGR5 BsAb).

The EGFR/LGR5 BsAb is preferably an anti-EGFR/anti-LGR5 bispecific monoclonal antibody (EGFR/LGR5 BsAb). The EGFR/LGR5 BsAb is preferably an anti-EGFR/anti-LGR5 heterodimeric bispecific antibody. The EGFR/LGR5 BsAb is preferably a human monoclonal antibody. The EGFR/LGR5 BsAb is preferably an IgG antibody. The EGFR/LGR5 BsAb is preferably an IgG1 antibody. The EGFR/LGR5 BsAb is preferably a full-length antibody. The EGFR/LGR5 BsAb is preferably an anti-EGFR/anti-LGR5 bispecific heterodimeric IgG1 monoclonal antibody. The EGFR/LGR5 BsAb is preferably an anti-EGFR/anti-LGR5 bispecific heterodimeric full-length human IgG1 monoclonal antibody.

The bispecific antibody preferably comprises a first binding (or variable) domain that binds an extracellular part of EGFR and a second binding or variable domain that binds an extracellular part of LGR5. The EGFR/LGR5 antibody as described herein is preferably a bispecific antibody having two variable domains, one that binds EGFR and another that binds LGR5 as described herein.

The binding (or variable) domain that binds human EGFR, is preferably a domain with a heavy chain variable region that comprises at least the CDR3 sequence of the VH of MF3755 as depicted in FIG. 1 or a CDR3 sequence that differs in at most three, preferably in at most two, preferably in no more than one amino acid from a CDR3 sequence of the VH of MF3755 as depicted in FIG. 1.

The binding (or variable domain) that binds human EGFR, is preferably a domain with a heavy chain variable region that comprises at least the CDR1, CDR2 and CDR3 sequences of the VH of MF3755 as depicted in FIG. 1; or the CDR1, CDR2 and CDR3 sequences of the VH of MF3755 as depicted in FIG. 1 with at most three, preferably at most two, preferably at most one amino acid substitutions.

The binding (or variable) domain that binds human EGFR, is preferably a domain with a heavy chain variable region that comprises the sequence of the VH chain of MF3755 as depicted in FIG. 1; or the amino acid sequence of the VH chain of MF3755 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3755.

The binding (or variable) domain that binds human EGFR, is preferably a domain with a heavy chain variable region that comprises at least the CDR3 sequence of the VH of MF4280 as depicted in FIG. 1 or a CDR3 sequence that differs in at most three, preferably in at most two, preferably in no more than one amino acid from a CDR3 sequence of the VH of MF4280 as depicted in FIG. 1.

The binding (or variable) domain that binds human EGFR, is preferably a domain with a heavy chain variable region that comprises at least the CDR1, CDR2 and CDR3 sequences of the VH of MF4280 as depicted in FIG. 1; or the CDR1, CDR2 and CDR3 sequences of the VH of MF4280 as depicted in FIG. 1 with at most three, preferably at most two, preferably at most one amino acid substitutions.

The antibody preferably comprises an EGFR binding domain comprising a heavy chain variable region (VH) that comprises the sequence of the VH chain of MF3370, MF3755, MF4280 or MF4289 as depicted in FIG. 1; or an EGFR binding domain comprising a heavy chain variable region (VH) that comprises the sequence of the VH chain of MF3370, MF3755, MF4280 or MF4289 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof.

The binding (or variable) domain that binds human EGFR, is preferably a domain with a heavy chain variable region that comprises the sequence of the VH chain of MF4280 as depicted in FIG. 1; or the amino acid sequence of the VH chain of MF4280 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF4280.

In one embodiment the invention provides a pharmaceutical composition comprising an antibody comprising a first binding domain binding to EGFR and a second binding domain binding to LGR5, wherein a heavy chain variable region of said domain comprises at least the CDR3 sequence of an EGFR specific heavy chain variable region selected from the group consisting of MF3370; MF3755; MF4280 or MF4289 as depicted in FIG. 1 or wherein a heavy chain variable region of said domain comprises a heavy chain CDR3 sequence that differs in at most three, preferably in at most two, preferably in no more than one amino acid from a CDR3 sequence of a VH selected from the group consisting of MF3370; MF3755; MF4280 or MF4289 as depicted in FIG. 1. Said variable domain preferably comprises a heavy chain variable region comprising at least the CDR3 sequence of MF3370; MF3755; MF4280 or MF4289 as depicted in FIG. 1.

Said binding (or variable) domain preferably comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 1, or heavy chain variable region comprising at least CDR1, CDR2 and CDR3 sequences that differ in at most three, preferably in at most two, preferably in at most one amino acid from the CDR1, CDR2 and CDR3 sequences of an EGFR specific heavy chain variable region selected from the group consisting of MF3370; MF3755; MF4280 or MF4289 as depicted in FIG. 1. Said domain preferably comprises a heavy chain variable region comprising at least the CDR1, CDR2 and CDR3 sequences of MF3370; MF3755; MF4280 or MF4289 as depicted in FIG. 1. A preferred heavy chain variable region is MF3755. Another preferred heavy chain variable region is MF4280.

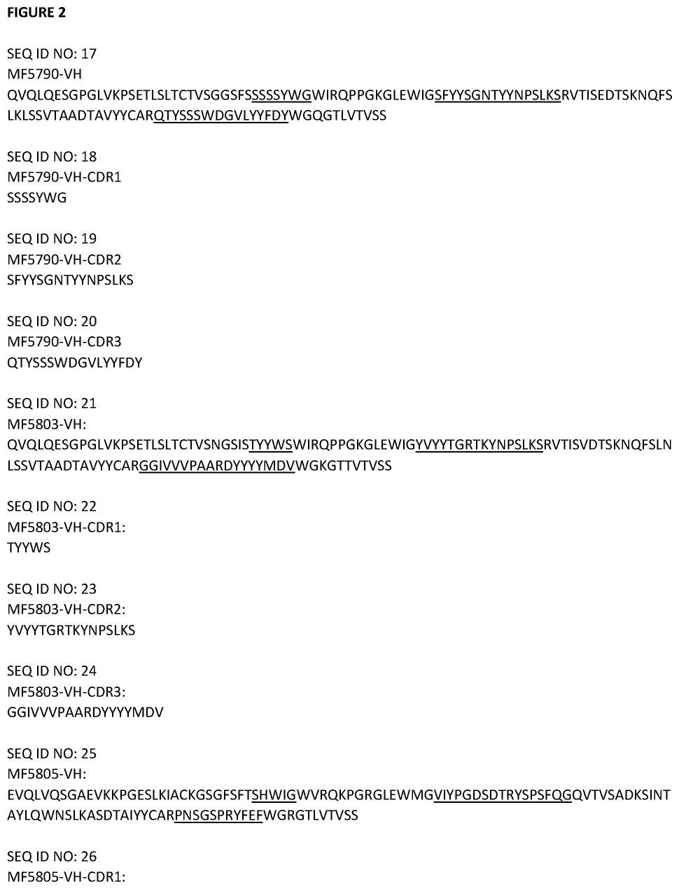

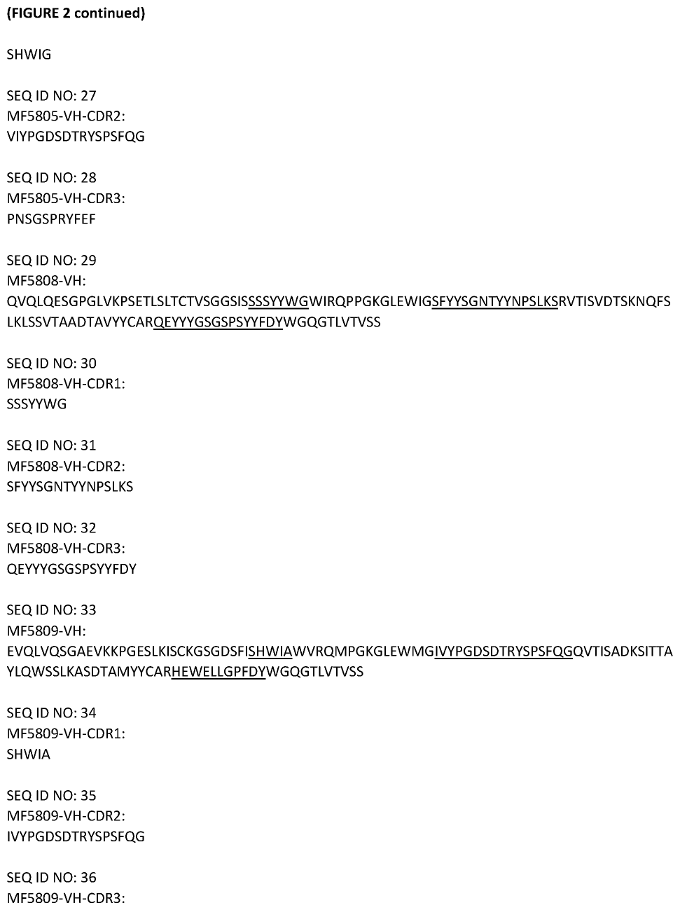

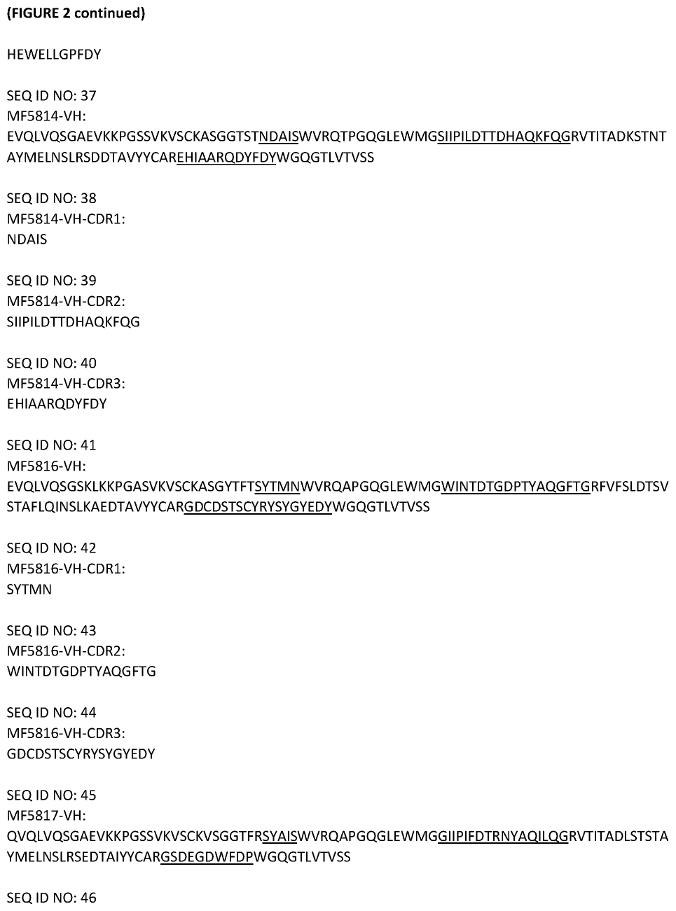

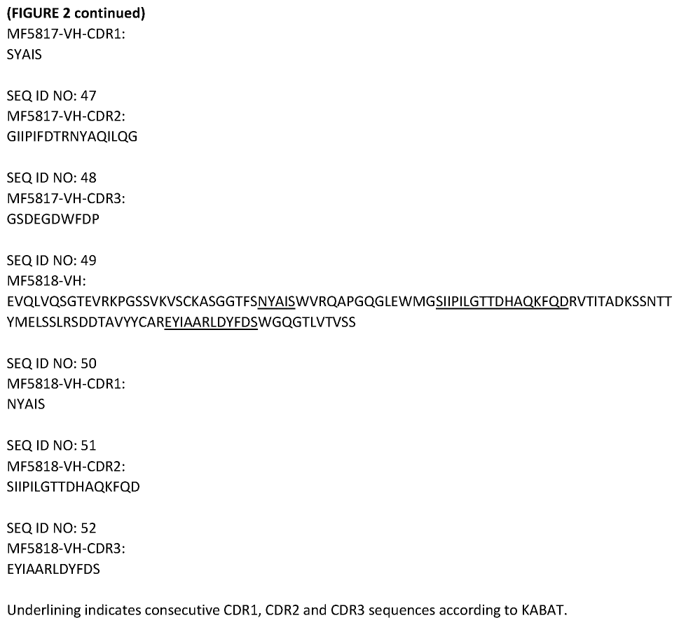

Preferably, the pharmaceutical composition comprises an antibody having a first binding domain binding to EGFR and a second binding domain binding to LGR5, wherein the EGFR binding domain has a CDR3, a CDR1, CDR2, and CDR3 and/or a VH sequence as indicated herein above preferably has a variable domain that binds LGR5 that comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 2 or a heavy chain CDR3 sequence that differs in at most three, preferably in at most two, preferably in no more than one amino acid from a CDR3 sequence of a VH selected from the group consisting of MF5790; MF5803; MF5805; MF5808; MF5809; MF5814; MF5816; MF5817; or MF5818 as depicted in FIG. 2. Said binding or variable domain preferably comprises a heavy chain variable region comprising at least the CDR3 sequence of MF5790; MF5803; MF5805; MF5808; MF5809; MF5814; MF5816; MF5817; or MF5818 as depicted in FIG. 2.

The LGR5 binding (or variable) domain preferably comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 2, or heavy chain CDR1, CDR2 and CDR3 sequences that differ in at most three, preferably in at most two, preferably in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 2. Said domain preferably comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 2. Preferred heavy chain variable regions are MF5790; MF5803; MF5814; MF5816; MF5817; or MF5818. Particularly preferred heavy chain variable regions are MF5790; MF5814; MF5816; and MF5818; preferably MF5814, MF5818 and MF5816, heavy chain variable region MF5816 is particularly preferred. Another preferred heavy chain variable region is MF5818.

Preferably, the antibody comprises an LGR5 binding domain comprising a heavy chain variable region that comprises the sequence of the VH chain of MF5790, MF5803, MF5805, MF5808, MF5809, MF5814, MF5816, MF5817 or MF5818 as depicted in FIG. 2; or the amino acid sequence of the VH chain of MF5790, MF5803, MF5805, MF5808, MF5809, MF5814, MF5816, MF5817 or MF5818 depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof.

VH chains of variable domains that bind EGFR or LGR5 can have one or more amino acid substitutions with respect to the sequence depicted in FIG. 1 or 2. A VH chain preferably has an amino acid sequence of an EGFR or LGR5 VH of FIG. 1 or 2, having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1 or 2.

Preferably, the mentioned at most 15, preferably 1, 2, 3, 4, 5, 6, 7, 8, 9 or 10 and preferably 1, 2, 3, 4 or 5 amino acid substitutions in a VH or VL as specified herein are preferably conservative amino acid substitutions. The amino acid insertions, deletions and substitutions in a VH or VL as specified herein are preferably not present in the CDR3 region. The mentioned amino acid insertions, deletions and substitutions are preferably also not present in the CDR1 and CDR2 regions. The mentioned amino acid insertions, deletions and substitutions are preferably also not present in the FR4 region.

The mentioned at most 15, preferably 1, 2, 3, 4, 5, 6, 7, 8, 9 or 10 and preferably 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

Preferably, the pharmaceutical composition comprises an antibody having a variable domain that binds an extracellular part of EGFR and a variable domain that binds an extracellular part of LGR5 that preferably comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5790 as depicted in FIG. 2; or
the amino acid sequence of VH chain MF5790 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Preferably, the pharmaceutical composition comprises an antibody having a variable domain that binds an extracellular part of EGFR and a variable domain that binds an extracellular part of LGR5 that preferably comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5816 as depicted in FIG. 2; or
the amino acid sequence of VH chain MF5816 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Preferably, the pharmaceutical composition comprises an antibody having a variable domain that binds an extracellular part of EGFR and a variable domain that binds an extracellular part of LGR5 that preferably comprises
the amino acid sequence of VH chain MF3755 as depicted in FIG. 1 or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5818 as depicted in FIG. 2; or
the amino acid sequence of VH chain MF5818 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Preferably, the pharmaceutical composition comprises an antibody having a variable domain that binds an extracellular part of EGFR and a variable domain that binds an extracellular part of LGR5 that preferably comprises
the amino acid sequence of VH chain MF4280 as depicted in FIG. 1; or
the amino acid sequence of VH chain MF4280 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5790 as depicted in FIG. 2; or
the amino acid sequence of VH chain MF5790 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Preferably, the pharmaceutical composition comprises an antibody having a variable domain that binds an extracellular part of EGFR and a variable domain that binds an extracellular part of LGR5 that preferably comprises
the amino acid sequence of VH chain MF4280 as depicted in FIG. 1; or
the amino acid sequence of VH chain MF4280 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5816 as depicted in FIG. 2; or
the amino acid sequence of VH chain MF5816 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Preferably, the pharmaceutical composition comprises an antibody having a variable domain that binds an extracellular part of EGFR and a variable domain that binds an extracellular part of LGR5 that preferably comprises
the amino acid sequence of VH chain MF4280 as depicted in FIG. 1 or
the amino acid sequence of VH chain MF4280 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and wherein the VH chain of the variable domain that binds LGR5 comprises
the amino acid sequence of VH chain MF5818 as depicted in FIG. 2; or
the amino acid sequence of VH chain MF5818 as depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The light chain variable regions of the VH/VL EGFR and LGR5 variable domains of the EGFR/LGR5 antibody may be the same or different.

The antibody preferably comprises a binding domain comprising a light chain variable region (VL) that comprises the sequence of the VL chain as depicted in FIG. 3; or a binding domain comprising a light chain variable region (VL) that comprises the sequence of the VL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

The antibody preferably comprises a binding domain comprising a CDR1, CDR2 and/or CDR3 amino acid sequence of the VL chain as depicted in FIG. 3; or a binding domain comprising a CDR1, CDR2 and/or CDR3 amino acid sequence of the VL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

The antibody preferably comprises a light chain constant region (CL) that comprises the sequence of the CL chain as depicted in FIG. 3; or a light chain constant region (CL) that comprises the sequence of the CL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

In some embodiments, the VL region of the VHNL EGFR variable domain of the EGFR/LGR5 antibody is similar to the VL region of the VH/VL LGR5 variable domain. In certain embodiments, VL regions in the first and second VH/VL variable domains are identical.

In certain embodiments, the light chain variable region of one or both VHNL variable domains of the EGFR/LGR5 antibody comprises a common light chain variable region. In some embodiments, the common light chain variable region of one or both VHNL variable domains comprises a germline IgVk1-39 variable region V-segment. In certain embodiment, the light chain variable region of one or both VHNL variable domains comprises the kappa light chain V-segment IgVk1-39*01. IgVk1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 2. The V-region can be combined with one of five J-regions. Preferred J-regions are jk1 and jk5, and the joined sequences are indicated as IGKV1-39/jk1 and IGKV1-39/jk5; alternative names are IgVk1-39*01/IGJK1*01 or IgVk1-39*01/IGJK5*01 (nomenclature according to the IMGT database worldwide web at imgt.org). In certain embodiments, the light chain variable region of one or both VH/VL variable domains comprises the kappa light chain IgVk1-39*01/IGJK1*01 or IgVk1-39*01/IGJK1*05 (described in FIG. 3).

In some embodiments, the light chain variable region of one or both VH/VL variable domains of the EGFR/LGR5 bispecific antibody comprises an LCDR1 comprising the amino acid sequence QSISSY (described in FIG. 3), an LCDR2 comprising the amino acid sequence AAS (described in FIG. 3), and an LCDR3 comprising the amino acid sequence QQSYSTP (described in FIG. 3) (i.e., the CDRs of IGKV1-39 according to IMGT). In some embodiments, the light chain variable region of one or both VH/VL variable domains of the EGFR/LGR5 antibody comprises an LCDR1 comprising the amino acid sequence QSISSY (described in FIG. 3), an LCDR2 comprising the amino acid sequence AASLQS (described in FIG. 3), and an LCDR3 comprising the amino acid sequence QQSYSTP (described in FIG. 3).

In some embodiments, the EGFR/LGR5 antibody comprises a light chain variable region comprising an amino acid sequence that is at least 90%, preferably at least 95%, more preferably at least 97%, more preferably at least 98%, more preferably at least 99% identical or 100% identical to the amino acid sequence set forth in FIG. 3. In some embodiments, the EGFR/LGR5 antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in FIG. 3.

For example, in some embodiments, the EGFR/LGR5 antibody can have from 0 to 10, preferably from 0 to 5 amino acid insertions, deletions, substitutions, additions or a combination thereof with respect to a sequence in FIG. 2. In some embodiments, the light chain variable region of one or both VH/VL variable domains of the EGFR/LGR5 antibody comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other embodiments, the light chain variable region of the EGFR/LGR5 antibody comprises the amino acid sequence of a sequence as depicted in FIG. 3. In certain embodiments, the EGFR/LGR5 antibody comprises identical VL regions. In one embodiment, the EGFR/LGR5 bispecific antibody comprises the amino acid sequence set forth in FIG. 3.

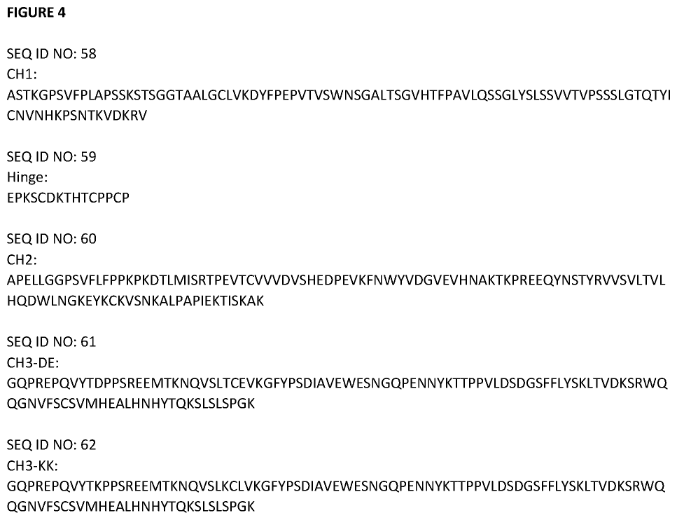

The EGFR/LGR5 BsAb is preferably an anti-EGFR/anti-LGR5 bispecific antibody comprising EGFR variable heavy chain MF3755 or MF4280 as depicted in FIG. 1, LGR5 variable heavy chain MF5790, MF5816 or MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1- N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 15 mM sodium citrate buffer; about 270 mM sucrose; about 0.2 mg/mL polysorbate 80; and water; wherein the composition has a pH of 6.7-6.9, preferably about 6.8.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium citrate buffer; about 280 mM sucrose; about 0.5 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM histidine buffer; about 290 mM maltose; about 0.2 mg/mL polysorbate 80; and water; wherein the composition has a pH of 6.2-6.4, preferably about 6.3.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 5 mM citrate phosphate buffer; about 290 mM sucrose; about 0.2 mg/mL polysorbate 80; and water; wherein the composition has a pH of 6.9-7.1, preferably about 7.0.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM sodium succinate buffer; about 280 mM sucrose; about 0.2 mg/mL polysorbate 80; and water; wherein the composition has a pH of 5.5-5.7, preferably about 5.6.

The EGFR/LGR5 BsAb is preferably an anti-EGFR/anti-LGR5 bispecific antibody comprising EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4. This particular anti-EGFR/anti-LGR5 bispecific antibody is preferably combined with any of the appended claims, any aspects of the invention defined herein, and/or any embodiments of the invention defined herein. This particular anti-EGFR/anti-LGR5 bispecific antibody is preferably combined with any of the embodiments A1-A127, B1-B128, C1-C128, D1-D36, E1-E16, F1-F16, G1-G16, H1-H12, I1-I16, J1-J24, K1-K32, L1-L64, M1-M1080 and N1-N2686. More preferably, the pharmaceutical composition of the present disclosure comprises or essentially consists of about 20 mg/ml of this particular anti-EGFR/anti-LGR5 bispecific antibody, about 10 mM histidine buffer; about 280 mM sucrose; about 0.5 mg/mL polysorbate 80; and water; wherein the composition has a pH of 5.8-6.0, preferably about 5.9.

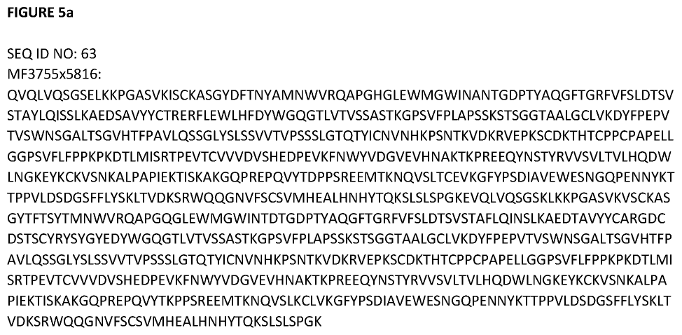

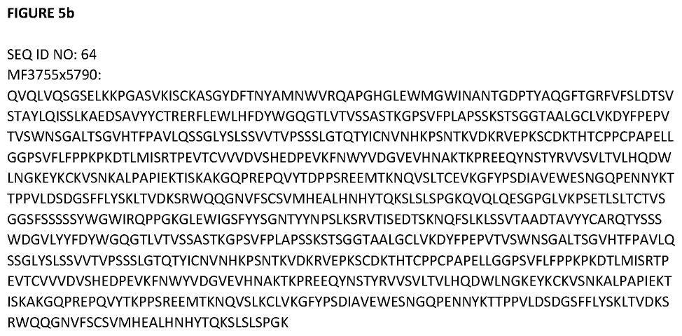

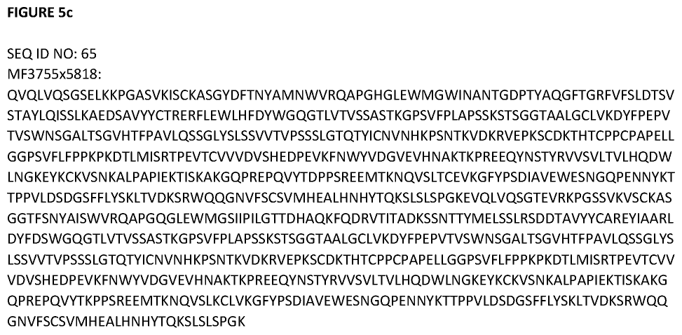

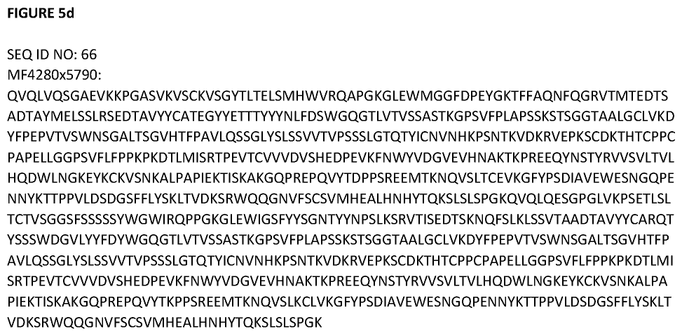

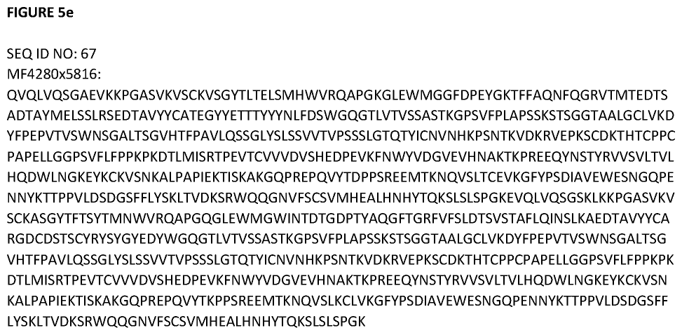

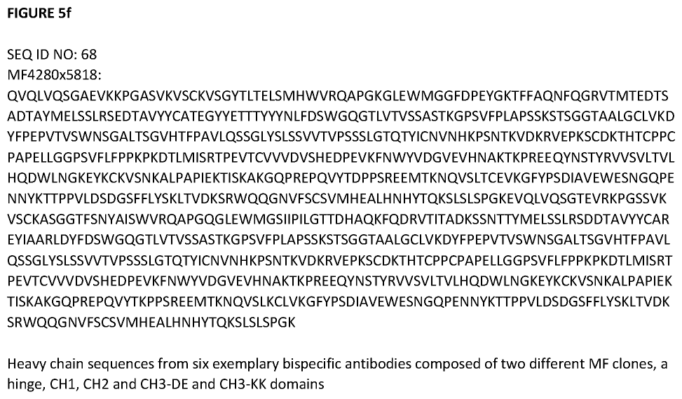

The EGFR/LGR5 BsAb is preferably an anti-EGFR/anti-LGR5 bispecific antibody comprising a heavy chain sequence corresponding to FIG. 5a, 5b, 5c, 5d, 5e or 5f or a variant thereof having at least 85% sequence identity to any of the sequences of FIGS. 5a, 5b, 5c, 5d, 5e or 5f. Also included herein are antibodies having at least 88% sequence identity to any of the sequences of FIGS. 5a, 5b, 5c, 5d, 5e or 5f. Preferably, the sequence identity is at least 92%, more preferably at least 93%, even more preferably at least 94 or 95% sequence identity.

Alternatively, the EGFR/LGR5 BsAb is preferably an anti-EGFR/anti-LGR5 bispecific antibody comprising a heavy chain sequence corresponding to FIG. 5a, 5b, 5c, 5d, 5e or 5f and a light chain sequence corresponding to FIG. 3 or a variant thereof having at least 85% sequence identity to any of the sequences of FIGS. 5a, 5b, 5c, 5d, 5e or 5f in combination with the sequences of FIG. 3. Also included herein are antibodies having at least 88% sequence identity to any of the sequences of FIGS. 5a, 5b, 5c, 5d, Se or 5f in combination with the sequences of FIG. 3. Preferably, the sequence identity is at least 92%, more preferably at least 93%, even more preferably at least 94 or 95% sequence identity any of the sequences of FIGS. 5a, 5b, 5c, 5d, 5e or 5f in combination with the sequences of FIG. 3. For the calculation of the sequence identity of the combination of sequences of FIGS. 3 and 5, these sequences are positioned to produce a full-length anti-EGFR/anti-LGR5 bispecific antibody sequence. Next, the sequence identity can be determined using the Vector NTI program as mentioned herein.

In an embodiment, the pharmaceutical composition comprises 1-150 mg/mL EGFR/LGR5 BsAb. In an embodiment, the pharmaceutical composition comprises 1-60 mg/mL EGFR/LGR5 BsAb. In an embodiment, the pharmaceutical composition comprises 1-25 mg/mL EGFR/LGR5 BsAb. In an embodiment, the pharmaceutical composition comprises 10-60 mg/mL EGFR/LGR5 BsAb. In an embodiment, the pharmaceutical composition comprises 10-30 mg/mL EGFR/LGR5 BsAb. In an embodiment, the pharmaceutical composition comprises 15-25 mg/mL EGFR/LGR5 BsAb. In an embodiment, the pharmaceutical composition comprises 20 mg/mL EGFR/LGR5 BsAb.

In an embodiment, the pharmaceutical composition comprises 1-150 mg/mL bispecific antibody binding both EGFR and LGR5. In an embodiment, the pharmaceutical composition comprises 1-60 mg/mL EGFR/LGR5 BSAB. In an embodiment, the pharmaceutical composition comprises 1-25 mg/mL EGFR/LGR5 BSAB. In an embodiment, the pharmaceutical composition comprises 10-60 mg/mL EGFR/LGR5 BsAb. In an embodiment, the pharmaceutical composition comprises 10-30 mg/mL EGFR/LGR5 BSAB. In an embodiment, the pharmaceutical composition comprises 15-25 mg/mL EGFR/LGR5 BSAB. In an embodiment, the pharmaceutical composition comprises 20 mg/mL EGFR/LGR5 BSAB.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

The pharmaceutical composition is preferably an aqueous composition comprising: 1-30 mg/mL bispecific antibody, functional part or derivative thereof, comprising a first binding domain binding to EGFR and a second binding domain binding to LGR5; 3-20 mM buffer system comprising a histidine buffer and/or a citrate buffer; 200-400 mM sugar and/or sugar alcohol component; and 0.01-2 mg/mL non-ionic surfactant; wherein the composition has a pH between 5.4 and 6.4.

The pharmaceutical composition is preferably an aqueous composition comprising: 15-25 mg/mL bispecific antibody, functional part or derivative thereof, comprising a first binding domain binding to EGFR and a second binding domain binding to LGR5; 4-12 mM buffer system comprising a histidine buffer and/or a citrate buffer; 250-350 mM sucrose; and 0.1-1.5 mg/mL polysorbate 80; wherein the composition has a pH between 5.4 and 6.4.

The pharmaceutical composition preferably is or consists of 20 mg/mL bispecific antibody, functional part or derivative thereof, comprising a first binding domain binding to EGFR and a second binding domain binding to LGR5; 10 mM of a histidine or citrate buffer system; 280 mM sucrose; 0.5 mg/mL polysorbate 80; and water; wherein the composition has a pH of 5.8-6.0.

The pharmaceutical composition preferably comprises a bispecific antibody, functional part or derivative thereof, comprising a first binding domain binding to EGFR and a second binding domain binding to LGR5; a buffer system comprising a histidine buffer and/or a citrate buffer, wherein the molar concentration ratio of buffer system (or combination of buffer systems) to antibody is between 21:1 and 146:1; a sugar and/or sugar alcohol component, wherein the molar concentration ratio of sugar and/or sugar alcohol component to antibody is between 1460:1 and 2920:1; and a non-ionic surfactant, wherein the molar concentration ratio of non-ionic surfactant to antibody is between 1:18 and 11:1; wherein the composition has a pH between 5.4 and 6.4.

The pharmaceutical composition preferably comprises: a bispecific antibody, functional part or derivative thereof, comprising a first binding domain binding to EGFR and a second binding domain binding to LGR5; a buffer system comprising a histidine buffer and/or a citrate buffer, wherein the molar concentration ratio of buffer system (or combination of buffer systems) to antibody is between 29:1 and 88:1; sucrose, wherein the molar concentration ratio of sucrose to antibody is between 1825:1 and 2555:1; and polysorbate 80, wherein the molar concentration ratio of polysorbate 80 to antibody is between 1:2 and 8.5:1; wherein the composition has a pH between 5.4 and 6.4.

The pharmaceutical composition preferably comprises or consists of a bispecific antibody, functional part or derivative thereof, comprising a first binding domain binding to EGFR and a second binding domain binding to LGR5; histidine buffer system, wherein the molar concentration ratio of histidine buffer system to antibody is 73:1; sucrose, wherein the molar concentration ratio of sucrose to antibody is 2040:1; polysorbate 80, wherein the molar concentration ratio of polysorbate 80 to antibody is 2.79:1; and optionally water; wherein the composition has a pH of 5.8-6.0.

Preferably, the pharmaceutical composition of the invention comprises a bispecific antibody comprising a first binding domain binding to EGFR and a second binding domain binding to LGR5, wherein the antibody comprises the VH amino acid sequence of MF3370 as depicted in FIG. 1 and/or the VH amino acid sequence of MF5790 as depicted in FIG. 2 and the VL amino acid sequence as depicted in FIG. 3. Preferably, the antibody further comprises the CH1, hinge, CH2 and CH3-DE and CH3-KK amino acid sequences set forth in FIG. 4.

Preferably, the pharmaceutical composition of the invention comprises a bispecific antibody comprising a first binding domain binding to EGFR and a second binding domain binding to LGR5, wherein the antibody comprises the VH amino acid sequence of MF3755 as depicted in FIG. 1 and/or the VH amino acid sequence of MF5816 as depicted in FIG. 2 and the VL amino acid sequence as depicted in FIG. 3. Preferably, the antibody further comprises the CH1, hinge, CH2 and CH3-DE and CH3-KK amino acid sequences set forth in FIG. 4.

Preferably, the pharmaceutical composition of the invention comprises a bispecific antibody comprising a first binding domain binding to EGFR and a second binding domain binding to LGR5, wherein the antibody comprises the VH amino acid sequence of MF4280 as depicted in FIG. 1 and/or the VH amino acid sequence of MF5809 as depicted in FIG. 2 and the VL amino acid sequence as depicted in FIG. 3. Preferably, the antibody further comprises the CH1, hinge, CH2 and CH3-DE and CH3-KK amino acid sequences set forth in FIG. 4.

Preferably, the pharmaceutical composition of the invention comprises a bispecific antibody comprising a first binding domain binding to EGFR and a second binding domain binding to LGR5, wherein the antibody comprises the VH amino acid sequence of MF4289 as depicted in FIG. 1 and/or the VH amino acid sequence of MF5818 as depicted in FIG. 2 and the VL amino acid sequence as depicted in FIG. 3. Preferably, the antibody further comprises the CH1, hinge, CH2 and CH3-DE and CH3-KK amino acid sequences set forth in FIG. 4.

### Diluent

The pharmaceutical composition preferably comprises a diluent. The composition may include one or more pharmaceutically acceptable diluents, or mixtures thereof. However, most preferably the composition is an aqueous composition. Most preferably the diluent is water, and preferably water alone. The water is preferably water for injection (WFI).

Preferably the diluent may constitute the balance of ingredients in any composition, for instance so that the weight percentages of all ingredients total 100%. Preferably any concentrations given herein in relation to any component of the composition represent concentrations of said component in (and preferably dissolved in) the diluent in admixture with any other components.

The composition of the invention is preferably a solution, and is preferably (substantially or entirely) free of particulates or precipitates.

In an embodiment, however, the pharmaceutical composition is either free of water or comprises at most 10 wt % water, suitably at most 5 wt % water, suitably at most 2 wt % water, suitably at most 1 wt % water. Such embodiments may be solid pharmaceutical compositions or lyophilised pharmaceutical compositions which may, for instance, be capable of reconstitution (suitably through the addition of water and/or other relevant diluents, e.g. intravenous fluids, e.g. saline solution) prior to use, administration, or (suitably short-term) storage. Such lyophilised formulations may be reconstituted to afford aqueous pharmaceutical compositions disclosed herein (e.g. with ingredients present or absent in the concentrations specified herein).

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Buffer System

The pharmaceutical composition preferably comprises a buffer system. The buffer system preferably acts as a pH buffer creating inertia to pH changes when small amounts of (strong) acid or (strong) base are added to (or generated within, potentially as a result of degradation of one or more of the components of the pharmaceutical composition, most likely the antibody thereof) the pharmaceutical composition. As such, the buffer system preferably maintains a substantially constant composition pH overtime, insodoing preferably mitigating against pH-triggered degradation pathways. Preferably the pharmaceutical composition is sufficiently buffered so as to resist pH changes of greater than or equal to 1 pH unit during 6 months storage at 2-8° C., preferably to resist pH changes of greater than or equal to 0.5 pH units, most preferably to resist pH changes of greater than oe equal to 0.2 pH units under the same storage conditions.

Preferably, the composition is a buffered solution whose pH is stabilised by a buffering agent (or a buffer system), preferably in combination with an acid/base conjugate of the buffering agent. As such, the biopharmaceutical composition preferably comprises a buffering agent as defined herein. Preferably, the composition additionally comprises an acid/base conjugate, wherein said acid/base conjugate corresponds to the conjugate acid or conjugate base of the buffering agent, depending on whether the buffering agent is itself a base or acid respectively. Collectively, the buffering agent and its acid/base conjugate may be considered a “buffer system”. The composition thus preferably comprises a “buffer system” (preferably comprising a buffering agent(s) and an acid/base conjugate(s) thereof), and any concentrations stipulated in relation to the buffer system generally relate to the combined concentrations of the buffering agent(s) and any acid/base conjugate(s) thereof. Any “buffer system” preferably comprises a weak acid and a weak base (see above definitions).

Preferably, the composition comprises an acid/base conjugate of the buffering agent. The combination of the buffering agent and its acid/base conjugate constitute a buffer system. Preferably, the composition comprises the buffering agent and its corresponding acid/base conjugate, preferably such that together the buffering agent and its acid/base conjugate are present at a level (i.e. absolute amount or concentration) and in a relative amount (or concentration) sufficient to provide the desired pH for the composition. The buffer system may be formed by simply mixing the buffering agent with its acid/base conjugate or may alternatively be formed by mixing an acid or base with either the buffering agent or its acid/base conjugate in order to form in situ the desired mixture of buffering agent and acid/base conjugate. For example, a buffer system may be formed by simply mixing an acetate buffering agent (e.g. sodium acetate) with its acid/base conjugate (i.e. acetic acid), preferably in a ratio appropriate to furnish the desired pH. Alternatively, a buffer system may be formed by adding a base (e.g. sodium hydroxide) to the acid/base conjugate (e.g. acetic acid) of the acetate buffering agent, preferably in an amount appropriate to furnish the desired pH and mixture of the buffering agent (e.g. sodium acetate) and corresponding acid/base conjugate (i.e. acetic acid). More preferably, a histidine buffer system is included which is formed by adding an acid (in particular HCl or histidine HCl) to histidine to obtain said histidine buffer system, preferably in an amount appropriate to furnish the desired pH and mixture of the buffering agent. Alternatively, either method of forming the buffer system may be employed, and pH may be judiciously adjusted by either adding further acid (preferably strong acid, such as HCl) or further base (preferably strong base, such as sodium hydroxide). Preferably, a histidine-acetate buffer is not included in the formulation of the present invention.

The buffer system is preferably a buffer system comprising one or more buffers. The buffer system is preferably a buffer system comprising at most one buffer. The buffer system is preferably a dual-buffer system (e.g. phosphate-citrate buffer). However, in some embodiments, the pharmaceutical composition may be characterised by an absence of a buffer system (or of any one or more of those specifically mentioned herein in relation to the buffer system). The pharmaceutical composition may, for example, be sufficiently stable without buffer where the antibody imparts sufficient self-buffering, which preferably occurs at higher concentrations of antibody.

The buffer system is preferably a buffer system selected from an inorganic buffer(s) (e.g. ammonium buffer, a bicarbonate buffer, a carbonate buffer, a borate buffer, a phosphate buffer), an organic buffer(s) (e.g. a carboxylate buffer, an organic ammonium buffer, an alkanolammonium buffer, a zwitterionic buffer, an amino acid buffer, an aromatic nitrogen buffer, a sugar buffer), and any combination thereof.

The buffer system preferably is, consists of, or comprises an amphoteric buffer system (e.g. amino acid buffer, e.g. histidine).

The buffer system preferably is, consists of, or comprises a monocarboxylate buffer system (e.g. an acetate buffer, a formate buffer, a lactate buffer, a salicylate buffer, a benzoate buffer).

The buffer system preferably is, consists of, or comprises a dicarboxylate buffer system (e.g. a succinate buffer, a maleate buffer, a malate buffer, a fumarate buffer, a tartrate buffer, an adipate buffer, a hexanedioate buffer).

The buffer system preferably is, consists of, or comprises a tricarboxylate buffer system (e.g. citrate buffer).

The buffer system preferably is, consists of, or comprises a zwitterionic buffer system (e.g. amino acid buffers, zwitterionic sulphonate buffers, e.g. a N-(2-Acetamido)-2-aminoethanesulfonic acid (ACES) buffer, a 2-Aminoethanesulfonic acid (AES) buffer, a N-(1,1-Dimethyl-2-hydroxyethyl)-3-amino-2-hydroxypropanesulfonic acid (AMPSO) buffer, a N,N-Bis-(2-hydroxyethyl)-2-aminoethanesulfonic acid (BES) buffer, a 3-(Cyclohexylamino)-propanesulfonic acid (CAPS)).

The buffer system preferably is, consists of, or comprises an amino acid buffer system (e.g. a histidine buffer, a glycine buffer, a lysine buffer, a glycylglycine buffer, a N-[Tris(hydroxymethyl)-methyl]-glycine (tricine) buffer, a glutamate buffer, an asparate buffer, a N,N-Bis(2-hydroxyethyl)-glycine (Bicine) buffer, a N-(2-Acetamido)-iminodiacetic acid (ADA) buffer).

The buffer system preferably is, consists of, or comprises an alkanolammonium buffer system (e.g. an aminomethylpropanol (AMP) buffer, an aminomethyl propanediol (AMPD) buffer, a tris(hydroxymethyl)aminomethane (Tris) buffer, a [Bis-(2-hydroxyethyl)-imino]-tris-(hydroxymethylmethane) (BIS-Tris) buffer, a 1,3-Bis[tris(hydroxymethyl)-methylamino]propane (Bis-tris propane) buffer).

The buffer system preferably is, consists of, or comprises a buffer system selected from the group consisting of a histidine buffer system, an acetate buffer system, a citrate buffer system, a succinate buffer system, a phosphate buffer system, a citrate-phosphate buffer system, a tris buffer system, and any combination thereof.

The buffer system preferably is, consists of, or comprises a buffer system selected from the group consisting of a histidine buffer system (preferably with the exception of a histidine-acetate buffer), a citrate buffer system, and any combination thereof.

The buffer system preferably is, consists of, or comprises a histidine buffer system.

The buffer system preferably is, consists of, or comprises an acetate buffer system.

The buffer system preferably is, consists of, or comprises a citrate buffer system.

The buffer system preferably is, consists of, or comprises a succinate buffer system.

The buffer system preferably is, consists of, or comprises a phosphate buffer system.

The buffer system preferably is, consists of, or comprises a citrate-phosphate buffer system.

The buffer system preferably is, consists of, or comprises a Tris buffer system.

In an embodiment, the pharmaceutical composition comprises 1-100 mM buffer system, in particular a histidine buffer system. In an embodiment, the pharmaceutical composition comprises 2-70 mM buffer system. In an embodiment, the pharmaceutical composition comprises 3-50 mM buffer system. In an embodiment, the pharmaceutical composition comprises 4-30 mM buffer system. In an embodiment, the pharmaceutical composition comprises 5-20 mM buffer system.

In an embodiment, the pharmaceutical composition comprises 5-14 mM buffer system. In an embodiment, the pharmaceutical composition comprises 10 mM buffer system. In an embodiment, the pharmaceutical composition comprises 3-12 mM buffer system. In an embodiment, the pharmaceutical composition comprises 5 mM buffer system.

In an embodiment, the pharmaceutical composition comprises a buffer system in a molar ratio of buffer system to antibody of from 4:1 to 1050:1. In an embodiment, the pharmaceutical composition comprises a buffer system in a molar ratio of buffer system to antibody of from 14:1 to 520:1. In an embodiment, the pharmaceutical composition comprises a buffer system in a molar ratio of buffer system to antibody of from 7:1 to 180:1. In an embodiment, the pharmaceutical composition comprises a buffer system in a molar ratio of buffer system to antibody of from 20:1 to 90:1.

In an embodiment, the pharmaceutical composition comprises 3-25 mM buffer system selected from the group consisting of a histidine buffer system, an acetate buffer system, a citrate buffer system, a succinate buffer system, a phosphate buffer system, a citrate-phosphate buffer system, a tris buffer system, and any combination thereof. In an embodiment, the pharmaceutical composition comprises 3-12 mM buffer system selected from the group consisting of a histidine buffer system, an acetate buffer system, a citrate buffer system, a succinate buffer system, a phosphate buffer system, a citrate-phosphate buffer system, a tris buffer system, and any combination thereof.

In an embodiment, the pharmaceutical composition comprises 3-25 mM buffer system selected from a histidine buffer system or a citrate buffer system. In an embodiment, the pharmaceutical composition comprises 3-12 mM buffer system selected from a histidine buffer system or a citrate buffer system.

In an embodiment, the pharmaceutical composition comprises 3-25 mM histidine buffer system. In an embodiment, the pharmaceutical composition comprises 3-12 mM histidine buffer system. In an embodiment, the pharmaceutical composition comprises 5 mM histidine buffer system. In an embodiment, the pharmaceutical composition comprises 10 mM histidine buffer system.

In an embodiment, the pharmaceutical composition comprises a histidine buffer system in a molar ratio of histidine buffer system to antibody of from 4:1 to 1050:1. In an embodiment, the pharmaceutical composition comprises a histidine buffer system in a molar ratio of histidine buffer system to antibody of from 14:1 to 520:1. In an embodiment, the pharmaceutical composition comprises a histidine buffer system in a molar ratio of histidine buffer system to antibody of from 7:1 to 180:1. In an embodiment, the pharmaceutical composition comprises a histidine buffer system in a molar ratio of histidine buffer system to antibody of from 20:1 to 90:1.

In an embodiment, the pharmaceutical composition comprises 3-25 mM acetate buffer system. In an embodiment, the pharmaceutical composition comprises 3-12 mM acetate buffer system.

In an embodiment, the pharmaceutical composition comprises an acetate buffer system in a molar ratio of acetate buffer system to antibody of from 4:1 to 1050:1. In an embodiment, the pharmaceutical composition comprises an acetate buffer system in a molar ratio of acetate buffer system to antibody of from 14:1 to 520:1. In an embodiment, the pharmaceutical composition comprises an acetate buffer system in a molar ratio of acetate buffer system to antibody of from 7:1 to 180:1. In an embodiment, the pharmaceutical composition comprises an acetate buffer system in a molar ratio of acetate buffer system to antibody of from 20:1 to 90:1.

In an embodiment, the pharmaceutical composition comprises 3-25 mM citrate buffer system. In an embodiment, the pharmaceutical composition comprises 3-12 mM citrate buffer system. In an embodiment, the pharmaceutical composition comprises 5 mM citrate buffer system. In an embodiment, the pharmaceutical composition comprises 10 mM citrate buffer system.

In an embodiment, the pharmaceutical composition comprises a citrate buffer system in a molar ratio of citrate buffer system to antibody of from 4:1 to 1050:1. In an embodiment, the pharmaceutical composition comprises a citrate buffer system in a molar ratio of citrate buffer system to antibody of from 14:1 to 520:1. In an embodiment, the pharmaceutical composition comprises a citrate buffer system in a molar ratio of citrate buffer system to antibody of from 7:1 to 180:1. In an embodiment, the pharmaceutical composition comprises a citrate buffer system in a molar ratio of citrate buffer system to antibody of from 20:1 to 90:1.

In an embodiment, the pharmaceutical composition comprises 3-25 mM succinate buffer system. In an embodiment, the pharmaceutical composition comprises 3-12 mM succinate buffer system.

In an embodiment, the pharmaceutical composition comprises a succinate buffer system in a molar ratio of succinate buffer system to antibody of from 4:1 to 1050:1. In an embodiment, the pharmaceutical composition comprises a succinate buffer system in a molar ratio of succinate buffer system to antibody of from 14:1 to 520:1. In an embodiment, the pharmaceutical composition comprises a succinate buffer system in a molar ratio of succinate buffer system to antibody of from 7:1 to 180:1. In an embodiment, the pharmaceutical composition comprises a succinate buffer system in a molar ratio of succinate buffer system to antibody of from 20:1 to 90:1.

In an embodiment, the pharmaceutical composition comprises 3-25 mM phosphate buffer system. In an embodiment, the pharmaceutical composition comprises 3-12 mM phosphate buffer system.

In an embodiment, the pharmaceutical composition comprises a phosphate buffer system in a molar ratio of phosphate buffer system to antibody of from 4:1 to 1050:1. In an embodiment, the pharmaceutical composition comprises a phosphate buffer system in a molar ratio of phosphate buffer system to antibody of from 14:1 to 520:1. In an embodiment, the pharmaceutical composition comprises a phosphate buffer system in a molar ratio of phosphate buffer system to antibody of from 7:1 to 180:1. In an embodiment, the pharmaceutical composition comprises a phosphate buffer system in a molar ratio of phosphate buffer system to antibody of from 20:1 to 90:1.

In an embodiment, the pharmaceutical composition comprises 3-25 mM citrate-phosphate buffer system. In an embodiment, the pharmaceutical composition comprises 3-12 mM citrate-phosphate buffer system.

In an embodiment, the pharmaceutical composition comprises a citrate-phosphate buffer system in a molar ratio of citrate-phosphate buffer system to antibody of from 4:1 to 1050:1. In an embodiment, the pharmaceutical composition comprises a citrate-phosphate buffer system in a molar ratio of citrate-phosphate buffer system to antibody of from 14:1 to 520:1. In an embodiment, the pharmaceutical composition comprises a citrate-phosphate buffer system in a molar ratio of citrate-phosphate buffer system to antibody of from 7:1 to 180:1. In an embodiment, the pharmaceutical composition comprises a citrate-phosphate buffer system in a molar ratio of citrate-phosphate buffer system to antibody of from 20:1 to 90:1.

In an embodiment, the pharmaceutical composition comprises 3-25 mM Tris buffer system. In an embodiment, the pharmaceutical composition comprises 3-12 mM Tris buffer system.

In an embodiment, the pharmaceutical composition comprises a Tris buffer system in a molar ratio of Tris buffer system to antibody of from 4:1 to 1050:1. In an embodiment, the pharmaceutical composition comprises a Tris buffer system in a molar ratio of Tris buffer system to antibody of from 14:1 to 520:1. In an embodiment, the pharmaceutical composition comprises a Tris buffer system in a molar ratio of Tris buffer system to antibody of from 7:1 to 180:1. In an embodiment, the pharmaceutical composition comprises a Tris buffer system in a molar ratio of Tris buffer system to antibody of from 20:1 to 90:1.

In an embodiment, the pharmaceutical composition comprises 3-25 mM histidine buffer system providing a composition pH of 5.4-7.1. In an embodiment, the pharmaceutical composition comprises 3-25 mM histidine buffer system providing a composition pH of 5.7-6.5.

In an embodiment, the pharmaceutical composition comprises 3-25 mM acetate buffer system providing a composition pH of 4-5.6.

In an embodiment, the pharmaceutical composition comprises 3-25 mM citrate buffer system providing a composition pH of 4.5-7.5. In an embodiment, the pharmaceutical composition comprises 3-25 mM citrate buffer system providing a composition pH of 6-7.5. In an embodiment, the pharmaceutical composition comprises 3-25 mM citrate buffer system providing a composition pH of 6.5-7.5.

In an embodiment, the pharmaceutical composition comprises 3-25 mM succinate buffer system providing a composition pH of 4-6.6.

In an embodiment, the pharmaceutical composition comprises 3-25 mM phosphate buffer system providing a composition pH of 5.8-8.1.

In an embodiment, the pharmaceutical composition comprises 3-25 mM citrate-phosphate buffer system providing a composition pH of 4-7.1.

In an embodiment, the pharmaceutical composition comprises 3-25 mM Tris buffer system providing a composition pH of 7-8.6.

Amounts and concentrations in relation to the buffer system relate to total amounts and concentrations of all buffer systems, unless only a single buffer system is stipulated. Concentrations of the or any particular buffer system can be replaced by molar ratios.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and N1-N2686.

pH

The pharmaceutical composition preferably has a pH of 4-8.5. Though the antibody within the pharmaceutical composition may tolerate and remain reasonably stable over a range of pH values, certain pHs are in the context of the present invention particularly advantageous, especially in the presence of particular excipients or combinations thereof.

In an embodiment, the pharmaceutical composition has a pH of 4.5-7.5. In an embodiment, the pharmaceutical composition has a pH of 5.0-6.7. In an embodiment, the pharmaceutical composition has a pH of 5.4-6.0. In an embodiment, the pharmaceutical composition has a pH of 5.4-5.6. In an embodiment, the pharmaceutical composition has a pH of 5.8-6.0. In an embodiment, the pharmaceutical composition has a pH of 5.9-6.1. In an embodiment, the pharmaceutical composition has a pH of 6.2-6.4. In an embodiment, the pharmaceutical composition has a pH of 6-7.5. In an embodiment, the pharmaceutical composition has a pH of 6.5-7.5. In an embodiment, the pharmaceutical composition has a pH of 6.8-7.2.

In a preferred embodiment, the pharmaceutical composition has a pH of 5.4-6.5 and comprises a histidine buffer system. In an other preferred embodiment, the pharmaceutical composition has a pH of 5.8-6.4 and comprises a histidine buffer system. In a more preferred embodiment, the pharmaceutical composition has a pH of 5.8-6.0 and comprises a histidine buffer system. In an other preferred embodiment, the pharmaceutical composition has a pH of 6.2-6.4 and comprises a histidine buffer system.

In a preferred embodiment, the pharmaceutical composition has a pH of 6-7.5 and comprises a citrate buffer system. In another preferred embodiment, the pharmaceutical composition has a pH of 6.5-7.5 and comprises a citrate buffer system. In another preferred embodiment, the pharmaceutical composition has a pH of 6.8-7.2 and comprises a citrate buffer system. In another preferred embodiment, the pharmaceutical composition has a pH of 6.9-7.1 and comprises a citrate buffer system.

In an embodiment, the buffer system has a pH of 4-8.5. In an embodiment, the buffer system has a pH of 4-8.5. In an embodiment, the buffer system has a pH of 4.5-7.5. In an embodiment, the buffer system has a pH of 5.0-6.7. In an embodiment, the buffer system has a pH of 5.4-6.5. In an embodiment, the buffer system has a pH of 5.4-6.0. In an embodiment, the buffer system has a pH of 5.4-5.6. In an embodiment, the buffer system has a pH of 5.8-6.4. In an embodiment, the buffer system has a pH of 5.8-6.0. In an embodiment, the buffer system has a pH of 5.9-6.1. In an embodiment, the buffer system has a pH of 6.2-6.4.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Salt Tonicifier

The pharmaceutical composition may preferably comprise a salt tonicifier (e.g. NaCl, or other such salts), preferably a non-buffering tonicifier (i.e. a tonicifier that does not impart a pH buffering effect, unlike buffering salts which may themselves to an extent contribute to overall tonicify). In the context of the present invention, salt tonicifiers may be used to adjust the osmolality of the pharmaceutical composition, for instance, to bring the osmolality within a preferred range (e.g. for isotonicity—e.g. an osmolality of 200-400 mOsmol/L, more preferably 250-350 mOsmol/L, most preferably 270-310 mOsmol/L), though osmolality is sometimes of lesser importance to intravenous compositions than subcutaneous compositions. Salt tonicifiers may also facilitate solubility of the antibody and/or other excipients within the pharmaceutical composition. Salt tonicifiers may also impart a stabilising effect towards the antibody of the pharmaceutical composition, for example, improving conformational stability (e.g. reducing protein unfolding events that can increase the likelihood of ultimate aggregation), reducing aggregation, and/or reducing fragmentation.

However, the pharmaceutical composition may be characterised by an absence of a (non-buffering) salt tonicifier (or of any one or more of those specifically mentioned herein in relation to the salt tonicifier). In particular, the pharmaceutical composition may be characterised by an absence of sodium chloride, and most preferably is. Where sodium chloride or a salt tonicifier is absent, other components within the pharmaceutical composition may contribute towards tonicity of the composition, such as the sugar component, buffer, or other tonicity-imparting components.

The salt tonicifier preferably is, consists of, or comprises a salt tonicifier selected from the group consisting of sodium chloride, potassium chloride, magnesium chloride, calcium chloride, zinc chloride, or any combination thereof. The salt tonicifier preferably is, consists of, or comprises sodium chloride. The salt tonicifier preferably is, consists of, or comprises potassium chloride. The salt tonicifier preferably is, consists of, or comprises magnesium chloride. The salt tonicifier preferably is, consists of, or comprises calcium chloride. The salt tonicifier preferably is, consists of, or comprises zinc chloride.

In an embodiment, the pharmaceutical composition comprises 3-200 mM salt tonicifier. In an embodiment, the pharmaceutical composition comprises 3-7 mM salt tonicifier. In an embodiment, the pharmaceutical composition comprises 5-100 mM salt tonicifier. In an embodiment, the pharmaceutical composition comprises 5-50 mM salt tonicifier. In an embodiment, the pharmaceutical composition comprises 100-200 mM salt tonicifier. In an embodiment, the pharmaceutical composition comprises 130-170 mM salt tonicifier. In an embodiment, the pharmaceutical composition comprises 140-160 mM salt tonicifier. In an embodiment, the pharmaceutical composition comprises a salt tonicifier in a molar ratio of salt tonicifier to antibody of from 7:1 to 3000:1. In an embodiment, the pharmaceutical composition comprises a salt tonicifier in a molar ratio of salt tonicifier to antibody of from 21:1 to 1500:1. In an embodiment, the pharmaceutical composition comprises a salt tonicifier in a molar ratio of salt tonicifier to antibody of from 340:1 to 2500:1. In an embodiment, the pharmaceutical composition comprises a salt tonicifier in a molar ratio of salt tonicifier to antibody of from 900:1 to 1300:1.

In an embodiment, the pharmaceutical composition comprises 4-160 mM salt tonicifier selected from the group consisting of sodium chloride, potassium chloride, magnesium chloride, calcium chloride, zinc chloride, or any combination thereof. In an embodiment, the pharmaceutical composition comprises 10-180 mM sodium chloride.

In an embodiment, the pharmaceutical composition comprises 50-170 mM sodium chloride. In an embodiment, the pharmaceutical composition comprises 140-160 mM sodium chloride. In an embodiment, the pharmaceutical composition comprises sodium chloride in a molar ratio of sodium chloride to antibody of from 7:1 to 3000:1. In an embodiment, the pharmaceutical composition comprises sodium chloride in a molar ratio of sodium chloride to antibody of from 21:1 to 1500:1. In an embodiment, the pharmaceutical composition comprises sodium chloride in a molar ratio of sodium chloride to antibody of from 340:1 to 2500:1. In an embodiment, the pharmaceutical composition comprises sodium chloride in a molar ratio of sodium chloride to antibody of from 900:1 to 1300:1. In an embodiment, the pharmaceutical composition comprises a sufficient concentration of sodium chloride to produce a 200-400 mOsmol/L composition. In an embodiment, the pharmaceutical composition comprises a sufficient concentration of sodium chloride to produce a 250-350 mOsmol/L composition. In an embodiment, the pharmaceutical composition comprises a sufficient concentration of sodium chloride to produce a 270-310 mOsmol/L composition.

Amounts and concentrations in relation to the salt tonicifier relate to total amounts and concentrations of all salt tonicifiers, unless only a single salt tonicifier is stipulated. Concentrations of the or any particular salt tonicifier can be replaced by molar ratios.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Sugar Component

The pharmaceutical composition preferably comprises a sugar component, preferably a non-reducing sugar component. In the context of the invention, a sugar component can serve one or more functions within the pharmaceutical composition. For instance, they may serve as a lyoprotectant during freeze drying. A sugar component may impart tonicity, for instance, to bring the osmolality within a desired range (e.g. for isotonicity—e.g. an osmolality of 200-400 mOsmol/L, more preferably 250-350 mOsmol/L, most preferably 270-310 mOsmol/L). Using sugar components to contribute towards tonicity can be particularly useful where high ionic strengths are less desirable. A sugar component may facilitate solubility of the antibody and/or other excipients within the pharmaceutical composition. A sugar component may impart a stabilising effect towards the antibody of the pharmaceutical composition, for example, improving conformational stability (e.g. reducing protein unfolding events that can increase the likelihood of ultimate aggregation), reducing aggregation, and/or reducing fragmentation.

Preferably, the sugar component is a single compound. Preferably, the sugar component is, consists of, or comprises one or more sugar(s) and/or sugar alcohol(s).

Preferably, the sugar component is, consists of, or comprises one or more sugar(s), most preferably only a single (i.e. at most one) sugar. Preferably, the sugar component is, consists of, or comprises a sugar. Preferably, the sugar component is, consists of, or comprises a sugar selected from the group consisting of a monosaccharide, a disaccharide, a polysaccharide, and a complex carbohydrate. Preferably, the sugar component is, consists of, or comprises a monosaccharide. More preferably, the sugar component is, consists of, or comprises a disaccharide. Preferably, the sugar component is, consists of, or comprises a disaccharide selected from the group consisting of sucrose, trehalose, maltose, and lactose. Preferably, the sugar component is, consists of, or comprises a disaccharide selected from maltose, trehalose and/or sucrose. Most preferably, the sugar component is, consists of, or comprises sucrose.

Preferably, the sugar component is, consists of, or comprises one or more sugar alcohol(s). Preferably, the sugar component is, consists of, or comprises a sugar alcohol. Preferably, the sugar component is, consists of, or comprises a (3-12 C) sugar alcohol. Preferably, the sugar component is, consists of, or comprises a (3-6 C) sugar alcohol. Preferably, the sugar component is, consists of, or comprises a (5-6 C) sugar alcohol. Preferably, the sugar component is, consists of, or comprises a sugar alcohol selected from mannitol or sorbitol.

Most preferably, the sugar component is, consists of, or comprises sucrose. Preferably, the sugar component is sucrose and only sucrose. In a particular embodiment, the pharmaceutical composition comprises sucrose in an amount of 20-400 mM, 50-350 mM, 100-320 mM, 170-310 mM or 220-320 mM, more preferably 250-300 mM sucrose. In a particular embodiment, the pharmaceutical composition comprises 275-295 mM sucrose. Most preferably, the pharmaceutical composition comprises 280 mM sucrose.

In a particular embodiment, the pharmaceutical composition comprises a sugar component in a molar ratio of sugar component, in particular sucrose, to antibody of from 100:1 to 5500:1. In a particular embodiment, the pharmaceutical composition comprises a sugar component in a molar ratio of sugar component to antibody of from 300:1 to 2600:1. In a particular embodiment, the pharmaceutical composition comprises a sugar component in a molar ratio of sugar component to antibody of from 600:1 to 4500:1. In a particular embodiment, the pharmaceutical composition comprises a sugar component in a molar ratio of sugar component to antibody of from 1800:1 to 2200:1.

In a particular embodiment, the pharmaceutical composition comprises 100-400 mM sugar component selected from the group consisting of sucrose, trehalose, mannitol, sorbitol, glucose, maltose, lactose, and any combination thereof. In a particular embodiment, the pharmaceutical composition comprises 100-400 mM sugar component selected from the group consisting of sucrose, trehalose, mannitol, and sorbitol. In a particular embodiment, the pharmaceutical composition comprises 100-400 mM sugar component selected from the group consisting of sucrose, mannitol, sorbitol, and maltose, most preferably sucrose.

In a particular embodiment, the pharmaceutical composition comprises 150-350 mM sucrose. In a particular embodiment, the pharmaceutical composition comprises 250-300 mM sucrose. In a particular embodiment, the pharmaceutical composition comprises 280 mM sucrose. In a particular embodiment, the pharmaceutical composition comprises 290 mM sucrose. In a particular embodiment, the pharmaceutical composition comprises sucrose in a molar ratio of sucrose to antibody of from 100:1 to 5500:1. In a particular embodiment, the pharmaceutical composition comprises sucrose in a molar ratio of sucrose to antibody of from 300:1 to 2600:1. In a particular embodiment, the pharmaceutical composition comprises sucrose in a molar ratio of sucrose to antibody of from 600:1 to 4500:1. In a particular embodiment, the pharmaceutical composition comprises sucrose in a molar ratio of sucrose to antibody of from 1800:1 to 2200:1.

In a particular embodiment, the pharmaceutical composition comprises 150-350 mM trehalose. In a particular embodiment, the pharmaceutical composition comprises 250-300 mM trehalose. In a particular embodiment, the pharmaceutical composition comprises trehalose in a molar ratio of trehalose to antibody of from 100:1 to 5500:1. In a particular embodiment, the pharmaceutical composition comprises trehalose in a molar ratio of trehalose to antibody of from 300:1 to 2600:1. In a particular embodiment, the pharmaceutical composition comprises trehalose in a molar ratio of trehalose to antibody of from 600:1 to 4500:1. In a particular embodiment, the pharmaceutical composition comprises trehalose in a molar ratio of trehalose to antibody of from 1800:1 to 2200:1. In many preferred embodiments, the sugar component is not trehalose. In many preferred embodiments, the sugar component is free of trehalose. In many preferred embodiments the sugar component neither is nor comprises trehalose, optionally unless at a concentration of 99 mM or less.

In a particular embodiment, the pharmaceutical composition comprises 150-350 mM mannitol. In a particular embodiment, the pharmaceutical composition comprises 250-300 mM mannitol. In a particular embodiment, the pharmaceutical composition comprises mannitol in a molar ratio of mannitol to antibody of from 100:1 to 5500:1. In a particular embodiment, the pharmaceutical composition comprises mannitol in a molar ratio of mannitol to antibody of from 300:1 to 2600:1. In a particular embodiment, the pharmaceutical composition comprises mannitol in a molar ratio of mannitol to antibody of from 600:1 to 4500:1. In a particular embodiment, the pharmaceutical composition comprises mannitol in a molar ratio of mannitol to antibody of from 1800:1 to 2200:1.

In a particular embodiment, the pharmaceutical composition comprises 150-350 mM sorbitol. In a particular embodiment, the pharmaceutical composition comprises 250-300 mM sorbitol. In a particular embodiment, the pharmaceutical composition comprises sorbitol in a molar ratio of sorbitol to antibody of from 100:1 to 5500:1. In a particular embodiment, the pharmaceutical composition comprises sorbitol in a molar ratio of sorbitol to antibody of from 300:1 to 2600:1. In a particular embodiment, the pharmaceutical composition comprises sorbitol in a molar ratio of sorbitol to antibody of from 600:1 to 4500:1. In a particular embodiment, the pharmaceutical composition comprises sorbitol in a molar ratio of sorbitol to antibody of from 1800:1 to 2200:1.

In a particular embodiment, the pharmaceutical composition comprises 150-350 mM glucose. In a particular embodiment, the pharmaceutical composition comprises 250-300 mM glucose.

In a particular embodiment, the pharmaceutical composition comprises 150-350 mM maltose. In a particular embodiment, the pharmaceutical composition comprises 250-300 mM maltose.

In a particular embodiment, the pharmaceutical composition comprises 150-350 mM lactose. In a particular embodiment, the pharmaceutical composition comprises 250-300 mM lactose.

Amounts and concentrations in relation to the sugar component relate to total amounts and concentrations of all sugar components, unless only a single sugar component is stipulated. Concentrations of the or any particular sugar component can be replaced by molar ratios.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Surfactant

The pharmaceutical composition preferably comprises a surfactant. A surfactant may facilitate dissolution of the antibody and/or other components within the pharmaceutical composition. In the context of the present invention, a surfactant may prevent aggregation of components present in the pharmaceutical composition to occur.

However, in some embodiments the pharmaceutical composition is characterised by an absence of a surfactant (or of any one or more of those specifically mentioned herein in relation to the surfactant).

Preferably, the surfactant is, consists of, or comprises at most one surfactant.

Preferably, the surfactant is, consists of, or comprises a non-ionic surfactant. Preferably, the surfactant is, consists of, or comprises a non-ionic surfactant selected from the group consisting of: a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, and any combination thereof. Preferably, the surfactant is, consists of, or comprises a non-ionic surfactant selected from the group consisting of a sorbitan ester (Span), an ethoxylated sorbitan ester (polysorbate, also known as Tween®), and a block alkoxylate. Preferably, the surfactant is, consists of, or comprises an ethoxylated fatty acid ester surfactant.

Preferably, the surfactant is, consists of, or comprises a surfactant selected from the group consisting of polysorbate 20, polysorbate 80, poloxamer 188, and any combination thereof. Preferably, the surfactant is, consists of, or comprises a surfactant selected from the group consisting of polysorbate 20, polysorbate 80, and poloxamer 188.

Preferably, the surfactant is, consists of, or comprises a surfactant selected from polysorbate 20 or polysorbate 80.

More preferably, the surfactant is, consists of, or comprises polysorbate 20.

Preferably, the surfactant is, consists of, or comprises poloxamer 188.

Preferably, the surfactant is, consists of, or comprises polysorbate 80. Preferably, the surfactant is polysorbate 80.

In an embodiment, the pharmaceutical composition comprises 0.01-2 mg/mL surfactant. In an embodiment, the pharmaceutical composition comprises 0.05-1.5 mg/mL surfactant. In an embodiment, the pharmaceutical composition comprises 0.1-1.3 mg/mL surfactant. In an embodiment, the pharmaceutical composition comprises 0.1-0.6 mg/mL surfactant. In an embodiment, the pharmaceutical composition comprises 0.1-0.3 mg/mL surfactant. In an embodiment, the pharmaceutical composition comprises 0.2 mg/mL surfactant. In an embodiment, the pharmaceutical composition comprises 0.2-0.8 mg/mL surfactant. In an embodiment, the pharmaceutical composition comprises 0.4-0.6 mg/mL surfactant. In an embodiment, the pharmaceutical composition comprises 0.5 mg/mL surfactant. In an embodiment, the pharmaceutical composition comprises 0.4-1.2 mg/mL surfactant. In an embodiment, the pharmaceutical composition comprises 0.9-1.1 mg/mL surfactant. In an embodiment, the pharmaceutical composition comprises 1 mg/mL surfactant.

In an embodiment, the pharmaceutical composition comprises a surfactant in a molar ratio of surfactant to antibody of from 1:11 to 17:1. In an embodiment, the pharmaceutical composition comprises a surfactant in a molar ratio of surfactant to antibody of from 1:4 to 9:1. In an embodiment, the pharmaceutical composition comprises a surfactant in a molar ratio of surfactant to antibody of from 1:1.5 to 7:1. In an embodiment, the pharmaceutical composition comprises a surfactant in a molar ratio of surfactant to antibody of from 2:1 to 4:1.

In an embodiment, the pharmaceutical composition comprises 0.01-1.5 mg/mL surfactant selected from the group consisting of polysorbate 20, polysorbate 80, poloxamer 188, and any combination thereof.

In an embodiment, the pharmaceutical composition comprises 0.05-1.5 mg/mL polysorbate 80. In an embodiment, the pharmaceutical composition comprises 0.1-0.6 mg/mL polysorbate 80. In an embodiment, the pharmaceutical composition comprises 0.1-0.3 mg/mL polysorbate 80. In an embodiment, the pharmaceutical composition comprises 0.4-1.2 mg/mL polysorbate 80. In an embodiment, the pharmaceutical composition comprises 0.4-0.6 mg/mL polysorbate 80. In an embodiment, the pharmaceutical composition comprises 0.5 mg/mL polysorbate 80. In an embodiment, the pharmaceutical composition comprises 0.9-1.1 mg/mL polysorbate 80. In an embodiment, the pharmaceutical composition comprises 1 mg/mL polysorbate 80. In an embodiment, the pharmaceutical composition comprises polysorbate 80 in a molar ratio of polysorbate 80 to antibody of from 1:11 to 17:1. In an embodiment, the pharmaceutical composition comprises polysorbate 80 in a molar ratio of polysorbate 80 to antibody of from 1:4 to 9:1. In an embodiment, the pharmaceutical composition comprises polysorbate 80 in a molar ratio of polysorbate 80 to antibody of from 1:1.5 to 7:1. In an embodiment, the pharmaceutical composition comprises polysorbate 80 in a molar ratio of polysorbate 80 to antibody of from 2:1 to 4:1.

In an embodiment, the pharmaceutical composition comprises 0.05-1.5 mg/mL polysorbate 20. In an embodiment, the pharmaceutical composition comprises 0.1-0.6 mg/mL polysorbate 20. In an embodiment, the pharmaceutical composition comprises 0.1-0.3 mg/mL polysorbate 20. In an embodiment, the pharmaceutical composition comprises 0.4-1.2 mg/mL polysorbate 20. In an embodiment, the pharmaceutical composition comprises 0.4-0.6 mg/mL polysorbate 20. In an embodiment, the pharmaceutical composition comprises 0.5 mg/mL polysorbate 20. In an embodiment, the pharmaceutical composition comprises 0.9-1.1 mg/mL polysorbate 20. In an embodiment, the pharmaceutical composition comprises 1 mg/mL polysorbate 20. In an embodiment, the pharmaceutical composition comprises polysorbate 20 in a molar ratio of polysorbate 20 to antibody of from 1:11 to 17:1. In an embodiment, the pharmaceutical composition comprises polysorbate 20 in a molar ratio of polysorbate 20 to antibody of from 1:4 to 9:1. In an embodiment, the pharmaceutical composition comprises polysorbate 20 in a molar ratio of polysorbate 20 to antibody of from 1:1.5 to 7:1. In an embodiment, the pharmaceutical composition comprises polysorbate 20 in a molar ratio of polysorbate 20 to antibody of from 2:1 to 4:1. In many preferred embodiments the surfactant neither is nor comprises polysorbate 20, optionally unless at a concentration of 0.4 mg/mL or greater.

In an embodiment, the pharmaceutical composition comprises 0.05-1.5 mg/mL poloxamer 188. In an embodiment, the pharmaceutical composition comprises 0.7-1.3 mg/mL poloxamer 188. In an embodiment, the pharmaceutical composition comprises 0.9-1.1 mg/mL poloxamer 188. In an embodiment, the pharmaceutical composition comprises 1 mg/mL poloxamer 188.

Amounts and concentrations in relation to the surfactant relate to total amounts and concentrations of all surfactants, unless only a single surfactant is stipulated. Concentrations of the or any particular surfactant can be replaced by molar ratios.

The embodiments and features disclosed in this section may preferably be combined with any of embodiments A1-A127, B1-B128, and C1-C128. The embodiments and features disclosed in this section may preferably be combined with any of embodiments D1-D36, E1-E16, F1-F16, G1-G16, H1-H12, I1-I16, J1-J24, K1-K32, L1-L64, M1-M1080, and N1-N2686.

### Antioxidant

The pharmaceutical composition may preferably comprise an antioxidant. However, in the most preferred embodiments, the pharmaceutical composition is characterised by an absence of an antioxidant (or of any one or more of those specifically mentioned herein in relation to the antioxidant). An antioxidant may inhibit oxidative degradation pathways open to the antibody and/or other components within the pharmaceutical composition. For instance, an antioxidant may inhibit oxidation of the antibody, for example by inhibiting oxidation of certain oxidizable amino acid residues within the antibody, and/or inhibit oxidative deamination pathways.

Preferably, the antioxidant is, consists of, or comprises an antioxidant selected from the group consisting of an amino acid or peptide antioxidant, a mineral antioxidant, a vitamin antioxidant, a carotenoid antioxidant, a polyphenol antioxidant, an aromatic or phenolic antioxidant, a chelating agent antioxidant, a thiol antioxidant, and any combination thereof. Preferably, the antioxidant is, consists of, or comprises an amino acid antioxidant. Preferably, the antioxidant is, consists of, or comprises an antioxidant selected from the group consisting of cysteine, methionine, N-acetyl-cysteine, glutathione, and any combination thereof. Preferably, the antioxidant is, consists of, or comprises an antioxidant selected from the group consisting of cysteine, methionine, and any combination thereof. Preferably, the antioxidant is, consists of, or comprises cysteine. Preferably, the antioxidant is, consists of, or comprises methionine.

In a particular embodiment, the pharmaceutical composition comprises 0.1-100 mM antioxidant. In a particular embodiment, the pharmaceutical composition comprises 1-50 mM antioxidant. In a particular embodiment, the pharmaceutical composition comprises 2-25 mM antioxidant. In a particular embodiment, the pharmaceutical composition comprises 3-12 mM antioxidant. In a particular embodiment, the pharmaceutical composition comprises 4-6 mM antioxidant.

In a particular embodiment, the pharmaceutical composition comprises an antioxidant in a molar ratio of antioxidant to antibody of from 2:1 to 750:1. In a particular embodiment, the pharmaceutical composition comprises an antioxidant in a molar ratio of antioxidant to antibody of from 271 to 400:1. In a particular embodiment, the pharmaceutical composition comprises an antioxidant in a molar ratio of antioxidant to antibody of from 7:1 to 180:1. In a particular embodiment, the pharmaceutical composition comprises an antioxidant in a molar ratio of antioxidant to antibody of from 20:1 to 90:1.

In a particular embodiment, the pharmaceutical composition comprises 1-50 mM antioxidant selected from the group consisting of cysteine, methionine, and any combination thereof. In a particular embodiment, the pharmaceutical composition comprises 2-25 mM cysteine. In a particular embodiment, the pharmaceutical composition comprises 3-12 mM cysteine. In a particular embodiment, the pharmaceutical composition comprises 4-6 mM cysteine. In a particular embodiment, the pharmaceutical composition comprises cysteine in a molar ratio of cysteine to antibody of from 2:1 to 750:1. In a particular embodiment, the pharmaceutical composition comprises cysteine in a molar ratio of cysteine to antibody of from 271 to 400:1. In a particular embodiment, the pharmaceutical composition comprises cysteine in a molar ratio of cysteine to antibody of from 7:1 to 180:1. In a particular embodiment, the pharmaceutical composition comprises cysteine in a molar ratio of cysteine to antibody of from 20:1 to 90:1.

In a particular embodiment, the pharmaceutical composition comprises 2-25 mM methionine. In a particular embodiment, the pharmaceutical composition comprises 3-12 mM methionine. In a particular embodiment, the pharmaceutical composition comprises 4-6 mM methionine. In a particular embodiment, the pharmaceutical composition comprises methionine in a molar ratio of methionine to antibody of from 2:1 to 750:1. In a particular embodiment, the pharmaceutical composition comprises methionine in a molar ratio of methionine to antibody of from 271 to 400:1. In a particular embodiment, the pharmaceutical composition comprises methionine in a molar ratio of methionine to antibody of from 7:1 to 180:1. In a particular embodiment, the pharmaceutical composition comprises methionine in a molar ratio of methionine to antibody of from 20:1 to 90:1.

Amounts and concentrations in relation to the antioxidant relate to total amounts and concentrations of all antioxidants, unless only a single antioxidant is stipulated. Concentrations of the or any particular antioxidant can be replaced by molar ratios.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Chelator

The pharmaceutical composition may suitably comprise a chelator. However, in the most preferred embodiments, the pharmaceutical composition is characterised by an absence of a chelator (or of any one or more of those specifically mentioned herein in relation to the chelator). In the context of the invention, a chelator such as EDTA (other chelators, are well known in the art and may serve a similar or identical function) may increase stability of the pharmaceutical composition, in particular inhibiting degradation of the antibody. For instance, chelators can reduce the likelihood of aggregation, and can also act as an antioxidant. Chelators can also sequester residual metals that may otherwise promoted degradation of the antibody and/or other components present within the pharmaceutical composition.

Suitably, the chelator is, consists of, or comprises EDTA.

In an embodiment, the pharmaceutical composition comprises 0.001-0.5 mM chelator. In an embodiment, the pharmaceutical composition comprises 0.01-0.2 mM chelator. In an embodiment, the pharmaceutical composition comprises 0.025-0.075 mM chelator.

In an embodiment, the pharmaceutical composition comprises a chelator in a molar ratio of chelator to antibody of from 1:420 to 8:1. In an embodiment, the pharmaceutical composition comprises a chelator in a molar ratio of chelator to antibody of from 1:140 to 4:1. In an embodiment, the pharmaceutical composition comprises a chelator in a molar ratio of chelator to antibody of from 1:16 to 1.1:1. In an embodiment, the pharmaceutical composition comprises a chelator in a molar ratio of chelator to antibody of from 1:6 to 1:1.5.

In an embodiment, the pharmaceutical composition comprises 0.001-0.5 mM EDTA. In an embodiment, the pharmaceutical composition comprises 0.01-0.2 mM EDTA. In an embodiment, the pharmaceutical composition comprises 0.025-0.075 mM EDTA. In an embodiment, the pharmaceutical composition comprises EDTA in a molar ratio of EDTA to antibody of from 1:420 to 8:1. In an embodiment, the pharmaceutical composition comprises EDTA in a molar ratio of EDTA to antibody of from 1:140 to 4:1. In an embodiment, the pharmaceutical composition comprises EDTA in a molar ratio of EDTA to antibody of from 1:16 to 1.1:1. In an embodiment, the pharmaceutical composition comprises EDTA in a molar ratio of EDTA to antibody of from 1:6 to 1:1.5.

Amounts and concentrations in relation to the chelator relate to total amounts and concentrations of all chelators, unless only a single chelator is stipulated. Concentrations of the or any particular chelator can be replaced by molar ratios.

The embodiments and features disclosed in this section may preferably be combined with any of embodiments A1-A127, B1-B128, C1-C128, D1-D36, E1-E16, F1-F16, G1-G16, H1-H12, I1-I16, J1-J24, K1-K32, L1-L64, M1-M1080, and
N1-N2686.

### Specific Embodiments

Sucrose has been found to be a particularly advantageous ingredient for use with the bispecific antibody of the invention binding both EGFR and LGR5, though trehalose and maltose are promising substitutes. As such:
D) the following numbered paragraphs D1 to D36 disclose specific embodiments of the invention, in which the pharmaceutical composition comprises (or consists of, optionally along with water for injection in the case of aqueous compositions) and is characterised by:
D1. an EGFR/LGR5 BsAb; and a sugar component in a molar ratio of sugar component to antibody of from 300:1 to 2600:1.
D2. 1-150 mg/mL EGFR/LGR5 BsAb; and a sugar component in a molar ratio of sugar component to antibody of from 300:1 to 2600:1.
D3. an EGFR/LGR5 BsAb; and a disaccharide.
D4. 1-150 mg/mL EGFR/LGR5 BsAb; and a disaccharide.
D5. an EGFR/LGR5 BsAb; and a disaccharide selected from the group consisting of sucrose, trehalose, and maltose.
D6. 1-150 mg/mL EGFR/LGR5 BsAb; and a disaccharide selected from the group consisting of sucrose, trehalose, and maltose.
D7. an EGFR/LGR5 BsAb; and sucrose.
D8. 1-150 mg/mL EGFR/LGR5 BsAb; and sucrose.
D9. an EGFR/LGR5 BsAb; and 150-350 mM sucrose.
D10. 1-150 mg/mL EGFR/LGR5 BsAb; and 150-350 mM sucrose.
D11. an EGFR/LGR5 BsAb; and trehalose.
D12. 1-150 mg/mL EGFR/LGR5 BsAb; and trehalose.
D13. an EGFR/LGR5 BsAb; and 150-350 mM trehalose.
D14. 1-150 mg/mL EGFR/LGR5 BsAb; and 150-350 mM trehalose.
D15. an EGFR/LGR5 BsAb; and maltose.
D16. 1-150 mg/mL EGFR/LGR5 BsAb; and maltose.
D17. an EGFR/LGR5 BsAb; and 150-350 mM maltose.
D18. 1-150 mg/mL EGFR/LGR5 BsAb; and 150-350 mM maltose.
D19. an EGFR/LGR5 BsAb; 4-30 mM buffer system; and a sugar component in a molar ratio of sugar component to antibody of from 300:1 to 2600:1.
D20. 1-150 mg/mL EGFR/LGR5 BsAb; 4-30 mM buffer system; and a sugar component in a molar ratio of sugar component to antibody of from 300:1 to 2600:1.
D21. an EGFR/LGR5 BsAb; 4-30 mM buffer system; and a disaccharide.
D22. 1-150 mg/mL EGFR/LGR5 BsAb; 4-30 mM buffer system; and a disaccharide.
D23. an EGFR/LGR5 BsAb; 4-30 mM buffer system; and a disaccharide selected from the group consisting of sucrose, trehalose, and maltose.
D24. 1-150 mg/mL EGFR/LGR5 BsAb; 4-30 mM buffer system; and a disaccharide selected from the group consisting of sucrose, trehalose, and maltose.
D25. an EGFR/LGR5 BsAb; 4-30 mM buffer system; and sucrose.
D26. 1-150 mg/mL EGFR/LGR5 BsAb; 4-30 mM buffer system; and sucrose.
D27. an EGFR/LGR5 BsAb; 4-30 mM buffer system; and 150-350 mM sucrose.
D28. 1-150 mg/mL EGFR/LGR5 BsAb; 4-30 mM buffer system; and 150-350 mM sucrose.
D29. an EGFR/LGR5 BsAb; 4-30 mM buffer system; and trehalose.
D30. 1-150 mg/mL EGFR/LGR5 BsAb; 4-30 mM buffer system; and trehalose.
D31. an EGFR/LGR5 BsAb; 4-30 mM buffer system; and 150-350 mM trehalose.
D32. 1-150 mg/mL EGFR/LGR5 BsAb; 4-30 mM buffer system; and 150-350 mM trehalose.
D33. an EGFR/LGR5 BsAb; 4-30 mM buffer system; and maltose.
D34. 1-150 mg/mL EGFR/LGR5 BsAb; 4-30 mM buffer system; and maltose.
D35. an EGFR/LGR5 BsAb; 4-30 mM buffer system; and 150-350 mM maltose.
D36. 1-150 mg/mL EGFR/LGR5 BsAb; 4-30 mM buffer system; and 150-350 mM maltose.

Sodium chloride has likewise been found to be a potentially advantageous ingredient for use with a bispecific antibody binding both EGFR and LGR5, though relative to sucrose is perhaps more advantageous at relatively higher pHs than for sucrose. As such:
E) the following numbered paragraphs E1 to E16 disclose specific embodiments of the invention, in which the pharmaceutical composition comprises (or consists of, optionally along with water for injection in the case of aqueous compositions) and is characterised by:
E1. an EGFR/LGR5 BsAb; and sodium chloride.
E2. 1-150 mg/mL EGFR/LGR5 BsAb; and sodium chloride.
E3. an EGFR/LGR5 BsAb; and 10-180 mM sodium chloride.
E4. 1-150 mg/mL EGFR/LGR5 BsAb; and 10-180 mM sodium chloride.
E5. an EGFR/LGR5 BsAb; 4-30 mM buffer system; and sodium chloride.
E6. 1-150 mg/mL EGFR/LGR5 BsAb; 4-30 mM buffer system; and sodium chloride.
E7. an EGFR/LGR5 BsAb; 4-30 mM buffer system; and 10-180 mM sodium chloride.
E8. 1-150 mg/mL EGFR/LGR5 BsAb; 4-30 mM buffer system; and 10-180 mM sodium chloride.
E9. an EGFR/LGR5 BsAb; a pH of 5.9-7.0; and sodium chloride.
E10. 1-150 mg/mL EGFR/LGR5 BsAb; a pH of 5.9-7.0; and sodium chloride.
E11. an EGFR/LGR5 BsAb; a pH of 5.9-7.0; and 10-180 mM sodium chloride.
E12. 1-150 mg/mL EGFR/LGR5 BsAb; a pH of 5.9-7.0; and 10-180 mM sodium chloride.
E13. an EGFR/LGR5 BsAb; 4-30 mM buffer system; a pH of 5.9-7.0; and sodium chloride.
E14. 1-150 mg/mL EGFR/LGR5 BsAb; 4-30 mM buffer system; a pH of 5.9-7.0; and sodium chloride.
E15. an EGFR/LGR5 BsAb; 4-30 mM buffer system; a pH of 5.9-7.0; and 10-180 mM sodium chloride.
E16. 1-150 mg/mL EGFR/LGR5 BsAb; 4-30 mM buffer system; a pH of 5.9-7.0; and 10-180 mM sodium chloride.

Surfactants do appear to enhance formulation stability, though polysorbate 80 and poloxamer 188 appear to be more beneficial from a freeze-thawing perspective, whist polysorbate 20 and polysorbate 80 provide the greatest improvements for stability against thermal stress, especially in the presence of buffers and sugar stabilisers, meaning polysorbate 80 has good versatilty for use. As such:
F) the following numbered paragraphs F1 to F16 disclose specific embodiments of the invention, in which the pharmaceutical composition comprises (or consists of, optionally along with water for injection in the case of aqueous compositions) and is characterised by:
F1. an EGFR/LGR5 BsAb; 4-30 mM buffer system; 50-350 mM sugar component; and 0.01-2 mg/mL surfactant.
F2. 1-150 mg/mL EGFR/LGR5 BsAb; 4-30 mM buffer system; 50-350 mM sugar component; and 0.01-2 mg/mL surfactant.
F3. an EGFR/LGR5 BsAb; 4-30 mM buffer system; 50-350 mM sugar component; and 0.05-1.5 mg/mL polysorbate 80.
F4. 1-150 mg/mL EGFR/LGR5 BsAb; 4-30 mM buffer system; 50-350 mM sugar component; and 0.05-1.5 mg/mL polysorbate 80.
F5. an EGFR/LGR5 BsAb; 4-30 mM buffer system; 50-350 mM sugar component; and 0.05-1.5 mg/mL polysorbate 20.
F6. 1-150 mg/mL EGFR/LGR5 BsAb; 4-30 mM buffer system; 50-350 mM sugar component; and 0.05-1.5 mg/mL polysorbate 20.
F7. an EGFR/LGR5 BsAb; 4-30 mM buffer system; 50-350 mM sugar component; and 0.05-1.5 mg/mL poloxamer 188.
F8. 1-150 mg/mL EGFR/LGR5 BsAb; 4-30 mM buffer system; 50-350 mM sugar component; and 0.05-1.5 mg/mL poloxamer 188.
F9. an EGFR/LGR5 BsAb; 4-30 mM buffer system; a pH of 4.4-7.4; 50-350 mM sugar component; and 0.01-2 mg/mL surfactant.
F10. 1-150 mg/mL EGFR/LGR5 BsAb; 4-30 mM buffer system; a pH of 4.4-7.4; 50-350 mM sugar component; and 0.01-2 mg/mL surfactant.
F11. an EGFR/LGR5 BsAb; 4-30 mM buffer system; a pH of 4.4-7.4; 50-350 mM sugar component; and 0.05-1.5 mg/mL polysorbate 80.
F12. 1-150 mg/mL EGFR/LGR5 BsAb; 4-30 mM buffer system; a pH of 4.4-7.4; 50-350 mM sugar component; and 0.05-1.5 mg/mL polysorbate 80.
F13. an EGFR/LGR5 BsAb; 4-30 mM buffer system; a pH of 4.4-7.4; 50-350 mM sugar component; and 0.05-1.5 mg/mL polysorbate 20.
F14. 1-150 mg/mL EGFR/LGR5 BsAb; 4-30 mM buffer system; a pH of 4.4-7.4; 50-350 mM sugar component; and 0.05-1.5 mg/mL polysorbate 20.
F15. an EGFR/LGR5 BsAb; 4-30 mM buffer system; a pH of 4.4-7.4; 50-350 mM sugar component; and 0.05-1.5 mg/mL poloxamer 188.
F16. 1-150 mg/mL EGFR/LGR5 BsAb; 4-30 mM buffer system; a pH of 4.4-7.4; 50-350 mM sugar component; and 0.05-1.5 mg/mL poloxamer 188.

An amino acid component, such as a non-antioxidant amino acid component, can contribute to formulation stability. In particular, proline, lysine, and glycine (and plausibly arginine also) may be advantageous, though proline and lysine tend to improve stability from a freeze-thaw perspective more than glycine, whereas glycine shows greater stabilising capacity with respect to thermal stress. As such:
G) the following numbered paragraphs G1 to G16 disclose specific embodiments of the invention, in which the pharmaceutical composition comprises (or consists of, optionally along with water for injection in the case of aqueous compositions) and is characterised by:
G1. an EGFR/LGR5 BsAb; 4-30 mM buffer system; 50-350 mM sugar component; and 5-150 mM amino acid component.
G2. 1-150 mg/mL EGFR/LGR5 BsAb; 4-30 mM buffer system; 50-350 mM sugar component; and 5-150 mM amino acid component.
G3. an EGFR/LGR5 BsAb; 4-30 mM buffer system; 50-350 mM sugar component; and 10-150 mM proline.
G4. 1-150 mg/mL EGFR/LGR5 BsAb; 4-30 mM buffer system; 50-350 mM sugar component; and 10-150 mM proline.
G5. an EGFR/LGR5 BsAb; 4-30 mM buffer system; 50-350 mM sugar component; and 10-150 mM glycine.
G6. 1-150 mg/mL EGFR/LGR5 BsAb; 4-30 mM buffer system; 50-350 mM sugar component; and 10-150 mM glycine.
G7. an EGFR/LGR5 BsAb; 4-30 mM buffer system; 50-350 mM sugar component; and 5-150 mM lysine.
G8. 1-150 mg/mL EGFR/LGR5 BsAb; 4-30 mM buffer system; 50-350 mM sugar component; and 5-150 mM lysine.
G9. an EGFR/LGR5 BsAb; 4-30 mM buffer system; a pH of 4.4-7.4; 50-350 mM sugar component; and 5-150 mM amino acid component.
G10. 1-150 mg/mL EGFR/LGR5 BsAb; 4-30 mM buffer system; a pH of 4.4-7.4; 50-350 mM sugar component; and 5-150 mM amino acid component.
G11. an EGFR/LGR5 BsAb; 4-30 mM buffer system; a pH of 4.4-7.4; 50-350 mM sugar component; and 10-150 mM proline.
G12. 1-150 mg/mL EGFR/LGR5 BsAb; 4-30 mM buffer system; a pH of 4.4-7.4; 50-350 mM sugar component; and 10-150 mM proline.
G13. an EGFR/LGR5 BsAb; 4-30 mM buffer system; a pH of 4.4-7.4; 50-350 mM sugar component; and 10-150 mM glycine.
G14. 1-150 mg/mL EGFR/LGR5 BsAb; 4-30 mM buffer system; a pH of 4.4-7.4; 50-350 mM sugar component; and 10-150 mM glycine.
G15. an EGFR/LGR5 BsAb; 4-30 mM buffer system; a pH of 4.4-7.4; 50-350 mM sugar component; and 5-150 mM lysine.
G16. 1-150 mg/mL EGFR/LGR5 BsAb; 4-30 mM buffer system; a pH of 4.4-7.4; 50-350 mM sugar component; and 5-150 mM lysine.

An antioxidant, such as an amino acid antioxidant, can contribute to formulation stability. In particular, methionine and cysteine may be advantageous, though cysteine slightly outperforms methionine in freeze thaw experiments, whereas methionine appears superior at resisting thermal stress. As such:
H) the following numbered paragraphs H1-H12 disclose specific embodiments of the invention, in which the pharmaceutical composition comprises (or consists of, optionally along with water for injection in the case of aqueous compositions) and is characterised by:
H1. an EGFR/LGR5 BsAb; 4-30 mM buffer system; 50-350 mM sugar component; and 1-50 mM antioxidant.
H2. 1-150 mg/mL EGFR/LGR5 BsAb; 4-30 mM buffer system; 50-350 mM sugar component; and 1-50 mM antioxidant.
H3. an EGFR/LGR5 BsAb; 4-30 mM buffer system; 50-350 mM sugar component; and 2-25 mM cysteine.
H4. 1-150 mg/mL EGFR/LGR5 BsAb; 4-30 mM buffer system; 50-350 mM sugar component; and 2-25 mM cysteine.
H5. an EGFR/LGR5 BsAb; 4-30 mM buffer system; 50-350 mM sugar component; and 2-25 mM methionine.
H6. 1-150 mg/mL EGFR/LGR5 BsAb; 4-30 mM buffer system; 50-350 mM sugar component; and 2-25 mM methionine.
H7. an EGFR/LGR5 BsAb; 4-30 mM buffer system; a pH of 4.4-7.4; 50-350 mM sugar component; and 1-50 mM antioxidant.
H8. 1-150 mg/mL EGFR/LGR5 BsAb; 4-30 mM buffer system; a pH of 4.4-7.4; 50-350 mM sugar component; and 1-50 mM antioxidant.
H9. an EGFR/LGR5 BsAb; 4-30 mM buffer system; a pH of 4.4-7.4; 50-350 mM sugar component; and 2-25 mM cysteine.
H10. 1-150 mg/mL EGFR/LGR5 BsAb; 4-30 mM buffer system; a pH of 4.4-7.4; 50-350 mM sugar component; and 2-25 mM cysteine.
H11. an EGFR/LGR5 BsAb; 4-30 mM buffer system; a pH of 4.4-7.4; 50-350 mM sugar component; and 2-25 mM methionine.
H12. 1-150 mg/mL EGFR/LGR5 BsAb; 4-30 mM buffer system; a pH of 4.4-7.4; 50-350 mM sugar component; and 2-25 mM methionine.

A chelator, such as EDTA, can contribute to formulation stability, in terms of resisting both freeze-thaw and thermal stress. As such:
I) the following numbered paragraphs 11 to 116 disclose specific embodiments of the invention, in which the pharmaceutical composition comprises (or consists of, optionally along with water for injection in the case of aqueous compositions) and is characterised by:
I1. an EGFR/LGR5 BsAb; 4-30 mM buffer system; 50-350 mM sugar component; and 0.001-0.5 mM chelator.
I2. 1-150 mg/mL EGFR/LGR5 BsAb; 4-30 mM buffer system; 50-350 mM sugar component; and 0.001-0.5 mM chelator.
I3. an EGFR/LGR5 BsAb; 4-30 mM buffer system; 50-350 mM sugar component; and 0.001-0.5 mM EDTA.
I4. 1-150 mg/mL EGFR/LGR5 BsAb; 4-30 mM buffer system; 50-350 mM sugar component; and 0.001-0.5 mM EDTA.
I5. an EGFR/LGR5 BsAb; 4-30 mM buffer system; a pH of 4.4-7.4; 50-350 mM sugar component; and 0.001-0.5 mM chelator.
I6. 1-150 mg/mL EGFR/LGR5 BsAb; 4-30 mM buffer system; a pH of 4.4-7.4; 50-350 mM sugar component; and 0.001-0.5 mM chelator.
I7. an EGFR/LGR5 BsAb; 4-30 mM buffer system; a pH of 4.4-7.4; 50-350 mM sugar component; and 0.001-0.5 mM EDTA.
I8. 1-150 mg/mL EGFR/LGR5 BsAb; 4-30 mM buffer system; a pH of 4.4-7.4; 50-350 mM sugar component; and 0.001-0.5 mM EDTA.
I9. an EGFR/LGR5 BsAb; 4-30 mM buffer system; 50-350 mM sugar component; 0.01-2 mg/mL surfactant; and 0.001-0.5 mM chelator.
I10. 1-150 mg/mL EGFR/LGR5 BsAb; 4-30 mM buffer system; 50-350 mM sugar component; 0.01-2 mg/mL surfactant; and 0.001-0.5 mM chelator.
I11. an EGFR/LGR5 BsAb; 4-30 mM buffer system; 50-350 mM sugar component; 0.01-2 mg/mL surfactant; and 0.001-0.5 mM EDTA.
I12. 1-150 mg/mL EGFR/LGR5 BsAb; 4-30 mM buffer system; 50-350 mM sugar component; 0.01-2 mg/mL surfactant; and 0.001-0.5 mM EDTA.
I113. an EGFR/LGR5 BsAb; 4-30 mM buffer system; a pH of 4.4-7.4; 50-350 mM sugar component; 0.01-2 mg/mL surfactant; and 0.001-0.5 mM chelator.
I14. 1-150 mg/mL EGFR/LGR5 BsAb; 4-30 mM buffer system; a pH of 4.4-7.4; 50-350 mM sugar component; 0.01-2 mg/mL surfactant; and 0.001-0.5 mM chelator.
I15. an EGFR/LGR5 BsAb; 4-30 mM buffer system; a pH of 4.4-7.4; 50-350 mM sugar component; 0.01-2 mg/mL surfactant; and 0.001-0.5 mM EDTA.
I16. 1-150 mg/mL EGFR/LGR5 BsAb; 4-30 mM buffer system; a pH of 4.4-7.4; 50-350 mM sugar component; 0.01-2 mg/mL surfactant; and 0.001-0.5 mM EDTA.

Certain metal (II) salts, especially alkaline earth metal salts, such as magnesium chloride and calcium chloride, can contribute to formulation stability, in terms of resisting freeze-thaw and/or thermal stress. As such:
J) the following numbered paragraphs J1 to J24 disclose specific embodiments of the invention, in which the pharmaceutical composition comprises (or consists of, optionally along with water for injection in the case of aqueous compositions) and is characterised by:
J1. an EGFR/LGR5 BsAb; 4-30 mM buffer system; 1-30 mM magnesium chloride; and 50-350 mM sugar component.
J2. 1-150 mg/mL EGFR/LGR5 BsAb; 4-30 mM buffer system; 1-30 mM magnesium chloride; and 50-350 mM sugar component.
J3. an EGFR/LGR5 BsAb; 4-30 mM buffer system; 1-30 mM calcium chloride; and 50-350 mM sugar component.
J4. 1-150 mg/mL EGFR/LGR5 BsAb; 4-30 mM buffer system; 1-30 mM calcium chloride; and 50-350 mM sugar component.
J5. an EGFR/LGR5 BsAb; 4-30 mM buffer system; a pH of 4.4-7.4; 1-30 mM magnesium chloride; and 50-350 mM sugar component.
J6. 1-150 mg/mL EGFR/LGR5 BsAb; 4-30 mM buffer system; a pH of 4.4-7.4; 1-30 mM magnesium chloride; and 50-350 mM sugar component.
J7. an EGFR/LGR5 BsAb; 4-30 mM buffer system; a pH of 4.4-7.4; 1-30 mM calcium chloride; and 50-350 mM sugar component.
J8. 1-150 mg/mL EGFR/LGR5 BsAb; 4-30 mM buffer system; a pH of 4.4-7.4; 1-30 mM calcium chloride; and 50-350 mM sugar component.
J9. an EGFR/LGR5 BsAb; 4-30 mM buffer system; 1-30 mM magnesium chloride; 50-350 mM sugar component; and 0.01-2 mg/mL surfactant.
J10. 1-150 mg/mL EGFR/LGR5 BsAb; 4-30 mM buffer system; 1-30 mM magnesium chloride; 50-350 mM sugar component; and 0.01-2 mg/mL surfactant.

J11. an EGFR/LGR5 BsAb; 4-30 mM buffer system; 1-30 mM calcium chloride; 50-350 mM sugar component; and 0.01-2 mg/mL surfactant.
J12. 1-150 mg/mL EGFR/LGR5 BsAb; 4-30 mM buffer system; 1-30 mM calcium chloride; 50-350 mM sugar component; and 0.01-2 mg/mL surfactant.
J13. an EGFR/LGR5 BsAb; 4-30 mM buffer system; 1-30 mM magnesium chloride; 50-350 mM sugar component; and 0.05-1.5 mg/mL polysorbate 80.
J14. 1-150 mg/mL EGFR/LGR5 BsAb; 4-30 mM buffer system; 1-30 mM magnesium chloride; 50-350 mM sugar component; and 0.05-1.5 mg/mL polysorbate 80.
J15. an EGFR/LGR5 BsAb; 4-30 mM buffer system; 1-30 mM calcium chloride; 50-350 mM sugar component; and 0.05-1.5 mg/mL polysorbate 80.
J16. 1-150 mg/mL EGFR/LGR5 BsAb; 4-30 mM buffer system; 1-30 mM calcium chloride; 50-350 mM sugar component; and 0.05-1.5 mg/mL polysorbate 80.
J17. an EGFR/LGR5 BsAb; 4-30 mM buffer system; a pH of 4.4-7.4; 1-30 mM magnesium chloride; 50-350 mM sugar component; and 0.01-2 mg/mL surfactant.
J18. 1-150 mg/mL EGFR/LGR5 BsAb; 4-30 mM buffer system; a pH of 4.4-7.4; 1-30 mM magnesium chloride; 50-350 mM sugar component; and 0.01-2 mg/mL surfactant.
J19. an EGFR/LGR5 BsAb; 4-30 mM buffer system; a pH of 4.4-7.4; 1-30 mM calcium chloride; 50-350 mM sugar component; and 0.01-2 mg/mL surfactant.
J20. 1-150 mg/mL EGFR/LGR5 BsAb; 4-30 mM buffer system; a pH of 4.4-7.4; 1-30 mM calcium chloride; 50-350 mM sugar component; and 0.01-2 mg/mL surfactant.
J21. an EGFR/LGR5 BsAb; 4-30 mM buffer system; a pH of 4.4-7.4; 1-30 mM magnesium chloride; 50-350 mM sugar component; and 0.05-1.5 mg/mL polysorbate 80.
J22. 1-150 mg/mL EGFR/LGR5 BsAb; 4-30 mM buffer system; a pH of 4.4-7.4; 1-30 mM magnesium chloride; 50-350 mM sugar component; and 0.05-1.5 mg/mL polysorbate 80.
J23. an EGFR/LGR5 BsAb; 4-30 mM buffer system; a pH of 4.4-7.4; 1-30 mM calcium chloride; 50-350 mM sugar component; and 0.05-1.5 mg/mL polysorbate 80.
J24. 1-150 mg/mL EGFR/LGR5 BsAb; 4-30 mM buffer system; a pH of 4.4-7.4; 1-30 mM calcium chloride; 50-350 mM sugar component; and 0.05-1.5 mg/mL polysorbate 80.

Buffers such as histidine, succinate, citrate, phosphate, citrate-phosphate, and acetate, perform particularly well at medium pHs, especially in the presence of a surfactant and a sugar component (especially sucrose, but also maltose).

As such:
K) the following numbered paragraphs K1 to K32 disclose specific embodiments of the invention, in which the pharmaceutical composition comprises (or consists of, optionally along with water for injection in the case of aqueous compositions) and is characterised by:
K1. an EGFR/LGR5 BsAb; 4-30 mM buffer system; 50-350 mM sugar component; and 0.01-2 mg/mL surfactant.
K2. 1-150 mg/mL EGFR/LGR5 BsAb; 4-30 mM buffer system; 50-350 mM sugar component; and 0.01-2 mg/mL surfactant.
K3. an EGFR/LGR5 BsAb; 3-25 mM histidine buffer system; 50-350 mM sugar component; and 0.01-2 mg/mL surfactant.
K4. 1-150 mg/mL EGFR/LGR5 BsAb; 3-25 mM histidine buffer system; 50-350 mM sugar component; and 0.01-2 mg/mL surfactant.
K5. an EGFR/LGR5 BsAb; 3-25 mM acetate buffer system; 50-350 mM sugar component; and 0.01-2 mg/mL surfactant.
K6. 1-150 mg/mL EGFR/LGR5 BsAb; 3-25 mM acetate buffer system; 50-350 mM sugar component; and 0.01-2 mg/mL surfactant.
K7. an EGFR/LGR5 BsAb; 3-25 mM citrate buffer system; 50-350 mM sugar component; and 0.01-2 mg/mL surfactant.
K8. 1-150 mg/mL EGFR/LGR5 BsAb; 3-25 mM citrate buffer system; 50-350 mM sugar component; and 0.01-2 mg/mL surfactant.
K9. an EGFR/LGR5 BsAb; 3-25 mM succinate buffer system; 50-350 mM sugar component; and 0.01-2 mg/mL surfactant.
K10. 1-150 mg/mL EGFR/LGR5 BsAb; 3-25 mM succinate buffer system; 50-350 mM sugar component; and 0.01-2 mg/mL surfactant.
K11. an EGFR/LGR5 BsAb; 3-25 mM phosphate buffer system; 50-350 mM sugar component; and 0.01-2 mg/mL surfactant.
K12. 1-150 mg/mL EGFR/LGR5 BsAb; 3-25 mM phosphate buffer system; 50-350 mM sugar component; and 0.01-2 mg/mL surfactant.
K13. an EGFR/LGR5 BsAb; 3-25 mM citrate-phosphate buffer system; 50-350 mM sugar component; and 0.01-2 mg/mL surfactant.
K14. 1-150 mg/mL EGFR/LGR5 BsAb; 3-25 mM citrate-phosphate buffer system; 50-350 mM sugar component; and 0.01-2 mg/mL surfactant.
K15. an EGFR/LGR5 BsAb; a Tris buffer system in a molar ratio of Tris buffer system to antibody of from 20:1 to 90:1; 50-350 mM sugar component; and 0.01-2 mg/mL surfactant.
K16. 1-150 mg/mL EGFR/LGR5 BsAb; a Tris buffer system in a molar ratio of Tris buffer system to antibody of from 20:1 to 90:1; 50-350 mM sugar component; and 0.01-2 mg/mL surfactant.
K17. an EGFR/LGR5 BsAb; 4-30 mM buffer system; a pH of 5.4-7.5; 50-350 mM sugar component; and 0.01-2 mg/mL surfactant.
K18. 1-150 mg/mL EGFR/LGR5 BsAb; 4-30 mM buffer system; a pH of 5.4-7.5; 50-350 mM sugar component; and 0.01-2 mg/mL surfactant.
K19. an EGFR/LGR5 BsAb; 3-25 mM histidine buffer system; a pH of 5.4-7.0; 50-350 mM sugar component; and 0.01-2 mg/mL surfactant.
K20. 1-150 mg/mL EGFR/LGR5 BsAb; 3-25 mM histidine buffer system; a pH of 5.4-7.0; 50-350 mM sugar component; and 0.01-2 mg/mL surfactant.
K21. an EGFR/LGR5 BsAb; 3-25 mM acetate buffer system; a pH of 5.4-7.5; 50-350 mM sugar component; and 0.01-2 mg/mL surfactant.
K22. 1-150 mg/mL EGFR/LGR5 BsAb; 3-25 mM acetate buffer system; a pH of 5.4-7.5; 50-350 mM sugar component; and 0.01-2 mg/mL surfactant.
K23. an EGFR/LGR5 BsAb; 3-25 mM citrate buffer system; a pH of 5.8-7.5; 50-350 mM sugar component; and 0.01-2 mg/mL surfactant.
K24. 1-150 mg/mL EGFR/LGR5 BsAb; 3-25 mM citrate buffer system; a pH of 5.8-7.5; 50-350 mM sugar component; and 0.01-2 mg/mL surfactant.
K25. an EGFR/LGR5 BsAb; 3-25 mM succinate buffer system; a pH of 5.4-7.5; 50-350 mM sugar component; and 0.01-2 mg/mL surfactant.
K26. 1-150 mg/mL EGFR/LGR5 BsAb; 3-25 mM succinate buffer system; a pH of 5.4-7.5; 50-350 mM sugar component; and 0.01-2 mg/mL surfactant.
K27. an EGFR/LGR5 BsAb; 3-25 mM phosphate buffer system; a pH of 5.4-7.5; 50-350 mM sugar component; and 0.01-2 mg/mL surfactant.
K28. 1-150 mg/mL EGFR/LGR5 BsAb; 3-25 mM phosphate buffer system; a pH of 5.4-7.5; 50-350 mM sugar component; and 0.01-2 mg/mL surfactant.
K29. an EGFR/LGR5 BsAb; 3-25 mM citrate-phosphate buffer system; a pH of 5.4-7.5; 50-350 mM sugar component; and 0.01-2 mg/mL surfactant.
K30. 1-150 mg/mL EGFR/LGR5 BsAb; 3-25 mM citrate-phosphate buffer system; a pH of 5.4-7.5; 50-350 mM sugar component; and 0.01-2 mg/mL surfactant.
K31. an EGFR/LGR5 BsAb; a Tris buffer system in a molar ratio of Tris buffer system to antibody of from 20:1 to 90:1; a pH of 5.4-7.5; 50-350 mM sugar component; and 0.01-2 mg/mL surfactant.
K32. 1-150 mg/mL EGFR/LGR5 BsAb; a Tris buffer system in a molar ratio of Tris buffer system to antibody of from 20:1 to 90:1; a pH of 5.4-7.5; 50-350 mM sugar component; and 0.01-2 mg/mL surfactant.

Buffers such as histidine, succinate, citrate, phosphate, citrate-phosphate, and acetate, perform particularly well at medium pHs for more concentrated antibody formulations, especially in the presence of a surfactant and a disaccharide sugar component (especially sucrose, but also maltose). As such:
L) the following numbered paragraphs L1 to L64 disclose specific embodiments of the invention, in which the pharmaceutical composition comprises (or consists of, optionally along with water for injection in the case of aqueous compositions) and is characterised by:
L1. 1-150 mg/mL EGFR/LGR5 BsAb; 4-30 mM buffer system; 50-350 mM disaccharide selected from the group consisting of sucrose, trehalose, and maltose; and 0.01-2 mg/mL surfactant.
L2. 1-150 mg/mL EGFR/LGR5 BsAb; 3-25 mM histidine buffer system; 50-350 mM disaccharide selected from the group consisting of sucrose, trehalose, and maltose; and 0.01-2 mg/mL surfactant.
L3. 1-150 mg/mL EGFR/LGR5 BsAb; 3-25 mM acetate buffer system; 50-350 mM disaccharide selected from the group consisting of sucrose, trehalose, and maltose; and 0.01-2 mg/mL surfactant.
L4. 1-150 mg/mL EGFR/LGR5 BsAb; 3-25 mM citrate buffer system; 50-350 mM disaccharide selected from the group consisting of sucrose, trehalose, and maltose; and 0.01-2 mg/mL surfactant.
L5. 1-150 mg/mL EGFR/LGR5 BsAb; 3-25 mM succinate buffer system; 50-350 mM disaccharide selected from the group consisting of sucrose, trehalose, and maltose; and 0.01-2 mg/mL surfactant.
L6. 1-150 mg/mL EGFR/LGR5 BsAb; 3-25 mM phosphate buffer system; 50-350 mM disaccharide selected from the group consisting of sucrose, trehalose, and maltose; and 0.01-2 mg/mL surfactant.
L7. 1-150 mg/mL EGFR/LGR5 BsAb; 3-25 mM citrate-phosphate buffer system; 50-350 mM disaccharide selected from the group consisting of sucrose, trehalose, and maltose; and 0.01-2 mg/mL surfactant.
L8. 1-150 mg/mL EGFR/LGR5 BsAb; a Tris buffer system in a molar ratio of Tris buffer system to antibody of from 20:1 to 90:1; 50-350 mM disaccharide selected from the group consisting of sucrose, trehalose, and maltose; and 0.01-2 mg/mL surfactant.
L9. 1-150 mg/mL EGFR/LGR5 BsAb; 4-30 mM buffer system; 250-300 mM sucrose; and 0.01-2 mg/mL surfactant.
L10. 1-150 mg/mL EGFR/LGR5 BsAb; 3-25 mM histidine buffer system; 250-300 mM sucrose; and 0.01-2 mg/mL surfactant.
L11. 1-150 mg/mL EGFR/LGR5 BsAb; 3-25 mM acetate buffer system; 250-300 mM sucrose; and 0.01-2 mg/mL surfactant.
L12. 1-150 mg/mL EGFR/LGR5 BsAb; 3-25 mM citrate buffer system; 250-300 mM sucrose; and 0.01-2 mg/mL surfactant.
L13. 1-150 mg/mL EGFR/LGR5 BsAb; 3-25 mM succinate buffer system; 250-300 mM sucrose; and 0.01-2 mg/mL surfactant.
L14. 1-150 mg/mL EGFR/LGR5 BsAb; 3-25 mM phosphate buffer system; 250-300 mM sucrose; and 0.01-2 mg/mL surfactant.
L15. 1-150 mg/mL EGFR/LGR5 BsAb; 3-25 mM citrate-phosphate buffer system; 250-300 mM sucrose; and 0.01-2 mg/mL surfactant.
L16. 1-150 mg/mL EGFR/LGR5 BsAb; a Tris buffer system in a molar ratio of Tris buffer system to antibody of from 20:1 to 90:1; 250-300 mM sucrose; and 0.01-2 mg/mL surfactant.
L17. 1-150 mg/mL EGFR/LGR5 BsAb; 4-30 mM buffer system; 50-350 mM disaccharide selected from the group consisting of sucrose, trehalose, and maltose; and 0.05-1.5 mg/mL polysorbate 80.
L18. 1-150 mg/mL EGFR/LGR5 BsAb; 3-25 mM histidine buffer system; 50-350 mM disaccharide selected from the group consisting of sucrose, trehalose, and maltose; and 0.05-1.5 mg/mL polysorbate 80.
L19. 1-150 mg/mL EGFR/LGR5 BsAb; 3-25 mM acetate buffer system; 50-350 mM disaccharide selected from the group consisting of sucrose, trehalose, and maltose; and 0.05-1.5 mg/mL polysorbate 80.
L20. 1-150 mg/mL EGFR/LGR5 BsAb; 3-25 mM citrate buffer system; 50-350 mM disaccharide selected from the group consisting of sucrose, trehalose, and maltose; and 0.05-1.5 mg/mL polysorbate 80.
L21. 1-150 mg/mL EGFR/LGR5 BsAb; 3-25 mM succinate buffer system; 50-350 mM disaccharide selected from the group consisting of sucrose, trehalose, and maltose; and 0.05-1.5 mg/mL polysorbate 80.
L22. 1-150 mg/mL EGFR/LGR5 BsAb; 3-25 mM phosphate buffer system; 50-350 mM disaccharide selected from the group consisting of sucrose, trehalose, and maltose; and 0.05-1.5 mg/mL polysorbate 80.
L23. 1-150 mg/mL EGFR/LGR5 BsAb; 3-25 mM citrate-phosphate buffer system; 50-350 mM disaccharide selected from the group consisting of sucrose, trehalose, and maltose; and 0.05-1.5 mg/mL polysorbate 80.
L24. 1-150 mg/mL EGFR/LGR5 BsAb; a Tris buffer system in a molar ratio of Tris buffer system to antibody of from 20:1 to 90:1; 50-350 mM disaccharide selected from the group consisting of sucrose, trehalose, and maltose; and 0.05-1.5 mg/mL polysorbate 80.
L25. 1-150 mg/mL EGFR/LGR5 BsAb; 4-30 mM buffer system; 250-300 mM sucrose; and 0.05-1.5 mg/mL polysorbate 80.
L26. 1-150 mg/mL EGFR/LGR5 BsAb; 3-25 mM histidine buffer system; 250-300 mM sucrose; and 0.05-1.5 mg/mL polysorbate 80.
L27. 1-150 mg/mL EGFR/LGR5 BsAb; 3-25 mM acetate buffer system; 250-300 mM sucrose; and 0.05-1.5 mg/mL polysorbate 80.
L28. 1-150 mg/mL EGFR/LGR5 BsAb; 3-25 mM citrate buffer system; 250-300 mM sucrose; and 0.05-1.5 mg/mL polysorbate 80.
L29. 1-150 mg/mL EGFR/LGR5 BsAb; 3-25 mM succinate buffer system; 250-300 mM sucrose; and 0.05-1.5 mg/mL polysorbate 80.
L30. 1-150 mg/mL EGFR/LGR5 BsAb; 3-25 mM phosphate buffer system; 250-300 mM sucrose; and 0.05-1.5 mg/mL polysorbate 80.
L31. 1-150 mg/mL EGFR/LGR5 BsAb; 3-25 mM citrate-phosphate buffer system; 250-300 mM sucrose; and 0.05-1.5 mg/mL polysorbate 80.
L32. 1-150 mg/mL EGFR/LGR5 BsAb; a Tris buffer system in a molar ratio of Tris buffer system to antibody of from 20:1 to 90:1; 250-300 mM sucrose; and 0.05-1.5 mg/mL polysorbate 80.
L33. 1-150 mg/mL EGFR/LGR5 BsAb; 4-30 mM buffer system; a pH of 5.4-7.2; 50-350 mM disaccharide selected from the group consisting of sucrose, trehalose, and maltose; and 0.01-2 mg/mL surfactant.
L34. 1-150 mg/mL EGFR/LGR5 BsAb; 3-25 mM histidine buffer system; a pH of 5.4-7.0; 50-350 mM disaccharide selected from the group consisting of sucrose, trehalose, and maltose; and 0.01-2 mg/mL surfactant.
L35. 1-150 mg/mL EGFR/LGR5 BsAb; 3-25 mM acetate buffer system; a pH of 5.4-7.2; 50-350 mM disaccharide selected from the group consisting of sucrose, trehalose, and maltose; and 0.01-2 mg/mL surfactant.
L36. 1-150 mg/mL EGFR/LGR5 BsAb; 3-25 mM citrate buffer system; a pH of 5.8-7.6; 50-350 mM disaccharide selected from the group consisting of sucrose, trehalose, and maltose; and 0.01-2 mg/mL surfactant.
L37. 1-150 mg/mL EGFR/LGR5 BsAb; 3-25 mM succinate buffer system; a pH of 5.4-7.2; 50-350 mM disaccharide selected from the group consisting of sucrose, trehalose, and maltose; and 0.01-2 mg/mL surfactant.
L38. 1-150 mg/mL EGFR/LGR5 BsAb; 3-25 mM phosphate buffer system; a pH of 5.4-7.2; 50-350 mM disaccharide selected from the group consisting of sucrose, trehalose, and maltose; and 0.01-2 mg/mL surfactant.
L39. 1-150 mg/mL EGFR/LGR5 BsAb; 3-25 mM citrate-phosphate buffer system; a pH of 5.8-7.6; 50-350 mM disaccharide selected from the group consisting of sucrose, trehalose, and maltose; and 0.01-2 mg/mL surfactant.
L40. 1-150 mg/mL EGFR/LGR5 BsAb; a Tris buffer system in a molar ratio of Tris buffer system to antibody of from 20:1 to 90:1; a pH of 5.4-7.2; 50-350 mM disaccharide selected from the group consisting of sucrose, trehalose, and maltose; and 0.01-2 mg/mL surfactant.
L41. 1-150 mg/mL EGFR/LGR5 BsAb; 4-30 mM buffer system; a pH of 5.4-7.2; 250-300 mM sucrose; and 0.01-2 mg/mL surfactant.
L42. 1-150 mg/mL EGFR/LGR5 BsAb; 3-25 mM histidine buffer system; a pH of 5.4-7.2; 250-300 mM sucrose; and 0.01-2 mg/mL surfactant.
L43. 1-150 mg/mL EGFR/LGR5 BsAb; 3-25 mM acetate buffer system; a pH of 5.4-7.2; 250-300 mM sucrose; and 0.01-2 mg/mL surfactant.
L44. 1-150 mg/mL EGFR/LGR5 BsAb; 3-25 mM citrate buffer system; a pH of 5.8-7.6; 250-300 mM sucrose; and 0.01-2 mg/mL surfactant.
L45. 1-150 mg/mL EGFR/LGR5 BsAb; 3-25 mM succinate buffer system; a pH of 5.4-7.2; 250-300 mM sucrose; and 0.01-2 mg/mL surfactant.
L46. 1-150 mg/mL EGFR/LGR5 BsAb; 3-25 mM phosphate buffer system; a pH of 5.4-7.2; 250-300 mM sucrose; and 0.01-2 mg/mL surfactant.
L47. 1-150 mg/mL EGFR/LGR5 BsAb; 3-25 mM citrate-phosphate buffer system; a pH of 5.4-7.2; 250-300 mM sucrose; and 0.01-2 mg/mL surfactant.
L48. 1-150 mg/mL EGFR/LGR5 BsAb; a Tris buffer system in a molar ratio of Tris buffer system to antibody of from 20:1 to 90:1; a pH of 5.4-7.2; 250-300 mM sucrose; and 0.01-2 mg/mL surfactant.
L49. 1-150 mg/mL EGFR/LGR5 BsAb; 4-30 mM buffer system; a pH of 5.4-7.2; 50-350 mM disaccharide selected from the group consisting of sucrose, trehalose, and maltose; and 0.05-1.5 mg/mL polysorbate 80.
L50. 1-150 mg/mL EGFR/LGR5 BsAb; 3-25 mM histidine buffer system; a pH of 5.4-7.2; 50-350 mM disaccharide selected from the group consisting of sucrose, trehalose, and maltose; and 0.05-1.5 mg/mL polysorbate 80.
L51. 1-150 mg/mL EGFR/LGR5 BsAb; 3-25 mM acetate buffer system; a pH of 5.4-7.2; 50-350 mM disaccharide selected from the group consisting of sucrose, trehalose, and maltose; and 0.05-1.5 mg/mL polysorbate 80.
L52. 1-150 mg/mL EGFR/LGR5 BsAb; 3-25 mM citrate buffer system; a pH of 5.8-7.8; 50-350 mM disaccharide selected from the group consisting of sucrose, trehalose, and maltose; and 0.05-1.5 mg/mL polysorbate 80.
L53. 1-150 mg/mL EGFR/LGR5 BsAb; 3-25 mM succinate buffer system; a pH of 5.4-7.2; 50-350 mM disaccharide selected from the group consisting of sucrose, trehalose, and maltose; and 0.05-1.5 mg/mL polysorbate 80.
L54. 1-150 mg/mL EGFR/LGR5 BsAb; 3-25 mM phosphate buffer system; a pH of 5.4-7.2; 50-350 mM disaccharide selected from the group consisting of sucrose, trehalose, and maltose; and 0.05-1.5 mg/mL polysorbate 80.
L55. 1-150 mg/mL EGFR/LGR5 BsAb; 3-25 mM citrate-phosphate buffer system; a pH of 5.4-7.2; 50-350 mM disaccharide selected from the group consisting of sucrose, trehalose, and maltose; and 0.05-1.5 mg/mL polysorbate 80.
L56. 1-150 mg/mL EGFR/LGR5 BsAb; a Tris buffer system in a molar ratio of Tris buffer system to antibody of from 20:1 to 90:1; a pH of 5.4-7.2; 50-350 mM disaccharide selected from the group consisting of sucrose, trehalose, and maltose; and 0.05-1.5 mg/mL polysorbate 80.
L57. 1-150 mg/mL EGFR/LGR5 BsAb; 4-30 mM buffer system; a pH of 5.4-7.2; 250-300 mM sucrose; and 0.05-1.5 mg/mL polysorbate 80.
L58. 1-150 mg/mL EGFR/LGR5 BsAb; 3-25 mM histidine buffer system; a pH of 5.4-7.2; 250-300 mM sucrose; and 0.05-1.5 mg/mL polysorbate 80.
L59. 1-150 mg/mL EGFR/LGR5 BsAb; 3-25 mM acetate buffer system; a pH of 5.4-7.2; 250-300 mM sucrose; and 0.05-1.5 mg/mL polysorbate 80.
L60. 1-150 mg/mL EGFR/LGR5 BsAb; 3-25 mM citrate buffer system; a pH of 5.8-7.8; 250-300 mM sucrose; and 0.05-1.5 mg/mL polysorbate 80.
L61. 1-150 mg/mL EGFR/LGR5 BsAb; 3-25 mM succinate buffer system; a pH of 5.4-7.2; 250-300 mM sucrose; and 0.05-1.5 mg/mL polysorbate 80.
L62. 1-150 mg/mL EGFR/LGR5 BsAb; 3-25 mM phosphate buffer system; a pH of 5.4-7.2; 250-300 mM sucrose; and 0.05-1.5 mg/mL polysorbate 80.
L63. 1-150 mg/mL EGFR/LGR5 BsAb; 3-25 mM citrate-phosphate buffer system; a pH of 5.4-7.2; 250-300 mM sucrose; and 0.05-1.5 mg/mL polysorbate 80.
L64. 1-150 mg/mL EGFR/LGR5 BsAb; a Tris buffer system in a molar ratio of Tris buffer system to antibody of from 20:1 to 90:1; a pH of 5.4-7.2; 250-300 mM sucrose; and 0.05-1.5 mg/mL polysorbate 80.

During judicious exploration of formulation space, histidine and citrate buffers were found to be particularly advantageous, especially in combination with a disaccharide, especially sucrose, and especially in combination with a surfactant, especially polysorbate 80. As such:
M) the following numbered paragraphs M1 to M1080 disclose specific embodiments of the invention, in which the pharmaceutical composition comprises (or consists of, optionally along with water for injection in the case of aqueous compositions) and is characterised by:
M1. 1-150 mg/mL EGFR/LGR5 BsAb; 2-70 mM buffer system; a pH of 5.4-7.2; 100-320 mM sugar component; and 0.01-2 mg/mL surfactant.
M2. 15-25 mg/mL EGFR/LGR5 BsAb; 2-70 mM buffer system; a pH of 5.4-7.2; 100-320 mM sugar component; and 0.01-2 mg/mL surfactant.
M3. 20 mg/mL EGFR/LGR5 BsAb; 2-70 mM buffer system; a pH of 5.4-7.2; 100-320 mM sugar component; and 0.01-2 mg/mL surfactant.
M4. 1-150 mg/mL EGFR/LGR5 BsAb; 3-12 mM histidine buffer system; a pH of 5.4-7.2; 100-320 mM sugar component; and 0.01-2 mg/mL surfactant.
M5. 15-25 mg/mL EGFR/LGR5 BsAb; 3-12 mM histidine buffer system; a pH of 5.4-7.2; 100-320 mM sugar component; and 0.01-2 mg/mL surfactant.
M6. 20 mg/mL EGFR/LGR5 BsAb; 3-12 mM histidine buffer system; a pH of 5.4-7.2; 100-320 mM sugar component; and 0.01-2 mg/mL surfactant.
M7. 1-150 mg/mL EGFR/LGR5 BsAb; 5 mM histidine buffer system; a pH of 5.4-7.2; 100-320 mM sugar component; and 0.01-2 mg/mL surfactant.
M8. 15-25 mg/mL EGFR/LGR5 BsAb; 5 mM histidine buffer system; a pH of 5.4-7.2; 100-320 mM sugar component; and 0.01-2 mg/mL surfactant.
M9. 20 mg/mL EGFR/LGR5 BsAb; 5 mM histidine buffer system; a pH of 5.4-7.2; 100-320 mM sugar component; and 0.01-2 mg/mL surfactant.
M10. 1-150 mg/mL EGFR/LGR5 BsAb; 10 mM histidine buffer system; a pH of 5.4-7.2; 100-320 mM sugar component; and 0.01-2 mg/mL surfactant.
M11. 15-25 mg/mL EGFR/LGR5 BsAb; 10 mM histidine buffer system; a pH of 5.4-7.2; 100-320 mM sugar component; and 0.01-2 mg/mL surfactant.
M12. 20 mg/mL EGFR/LGR5 BsAb; 10 mM histidine buffer system; a pH of 5.4-7.2; 100-320 mM sugar component; and 0.01-2 mg/mL surfactant.
M13. 1-150 mg/mL EGFR/LGR5 BsAb; 3-12 mM citrate buffer system; a pH of 5.8-7.8; 100-320 mM sugar component; and 0.01-2 mg/mL surfactant.
M14. 15-25 mg/mL EGFR/LGR5 BsAb; 3-12 mM citrate buffer system; a pH of 5.8-7.8; 100-320 mM sugar component; and 0.01-2 mg/mL surfactant.
M15. 20 mg/mL EGFR/LGR5 BsAb; 3-12 mM citrate buffer system; a pH of 5.8-7.8; 100-320 mM sugar component; and 0.01-2 mg/mL surfactant.
M16. 1-150 mg/mL EGFR/LGR5 BsAb; 10 mM citrate buffer system; a pH of 5.8-7.8; 100-320 mM sugar component; and 0.01-2 mg/mL surfactant.
M17. 15-25 mg/mL EGFR/LGR5 BsAb; 10 mM citrate buffer system; a pH of 5.8-7.8; 100-320 mM sugar component; and 0.01-2 mg/mL surfactant.
M18. 20 mg/mL EGFR/LGR5 BsAb; 10 mM citrate buffer system; a pH of 5.8-7.8; 100-320 mM sugar component; and 0.01-2 mg/mL surfactant.
M19. 1-150 mg/mL EGFR/LGR5 BsAb; 2-70 mM buffer system; a pH of 5.8-6.0; 100-320 mM sugar component; and 0.01-2 mg/mL surfactant.
M20. 15-25 mg/mL EGFR/LGR5 BsAb; 2-70 mM buffer system; a pH of 5.8-6.0; 100-320 mM sugar component; and 0.01-2 mg/mL surfactant.
M21. 20 mg/mL EGFR/LGR5 BsAb; 2-70 mM buffer system; a pH of 5.8-6.0; 100-320 mM sugar component; and 0.01-2 mg/mL surfactant.
M22. 1-150 mg/mL EGFR/LGR5 BsAb; 3-12 mM histidine buffer system; a pH of 5.8-6.0; 100-320 mM sugar component; and 0.01-2 mg/mL surfactant.
M23. 15-25 mg/mL EGFR/LGR5 BsAb; 3-12 mM histidine buffer system; a pH of 5.8-6.0; 100-320 mM sugar component; and 0.01-2 mg/mL surfactant.
M24. 20 mg/mL EGFR/LGR5 BsAb; 3-12 mM histidine buffer system; a pH of 5.8-6.0; 100-320 mM sugar component; and 0.01-2 mg/mL surfactant.
M25. 1-150 mg/mL EGFR/LGR5 BsAb; 5 mM histidine buffer system; a pH of 5.8-6.0; 100-320 mM sugar component; and 0.01-2 mg/mL surfactant.
M26. 15-25 mg/mL EGFR/LGR5 BsAb; 5 mM histidine buffer system; a pH of 5.8-6.0; 100-320 mM sugar component; and 0.01-2 mg/mL surfactant.
M27. 20 mg/mL EGFR/LGR5 BsAb; 5 mM histidine buffer system; a pH of 5.8-6.0; 100-320 mM sugar component; and 0.01-2 mg/mL surfactant.
M28. 1-150 mg/mL EGFR/LGR5 BsAb; 10 mM histidine buffer system; a pH of 5.8-6.0; 100-320 mM sugar component; and 0.01-2 mg/mL surfactant.
M29. 15-25 mg/mL EGFR/LGR5 BsAb; 10 mM histidine buffer system; a pH of 5.8-6.0; 100-320 mM sugar component; and 0.01-2 mg/mL surfactant.
M30. 20 mg/mL EGFR/LGR5 BsAb; 10 mM histidine buffer system; a pH of 5.8-6.0; 100-320 mM sugar component; and 0.01-2 mg/mL surfactant.
M31. 1-150 mg/mL EGFR/LGR5 BsAb; 3-12 mM citrate buffer system; a pH of 5.8-7; 100-320 mM sugar component; and 0.01-2 mg/mL surfactant.
M32. 15-25 mg/mL EGFR/LGR5 BsAb; 3-12 mM citrate buffer system; a pH of 5.8-7; 100-320 mM sugar component; and 0.01-2 mg/mL surfactant.
M33. 20 mg/mL EGFR/LGR5 BsAb; 3-12 mM citrate buffer system; a pH of 5.8-7; 100-320 mM sugar component; and 0.01-2 mg/mL surfactant.
M34. 1-150 mg/mL EGFR/LGR5 BsAb; 10 mM citrate buffer system; a pH of 5.8-7; 100-320 mM sugar component; and 0.01-2 mg/mL surfactant.
M35. 15-25 mg/mL EGFR/LGR5 BsAb; 10 mM citrate buffer system; a pH of 5.8-7; 100-320 mM sugar component; and 0.01-2 mg/mL surfactant.
M36. 20 mg/mL EGFR/LGR5 BsAb; 10 mM citrate buffer system; a pH of 5.8-7; 100-320 mM sugar component; and 0.01-2 mg/mL surfactant.
M37. 1-150 mg/mL EGFR/LGR5 BsAb; 2-70 mM buffer system; a pH of 6.2-6.4; 100-320 mM sugar component; and 0.01-2 mg/mL surfactant.
M38. 15-25 mg/mL EGFR/LGR5 BsAb; 2-70 mM buffer system; a pH of 6.2-6.4; 100-320 mM sugar component; and 0.01-2 mg/mL surfactant.
M39. 20 mg/mL EGFR/LGR5 BsAb; 2-70 mM buffer system; a pH of 6.2-6.4; 100-320 mM sugar component; and 0.01-2 mg/mL surfactant.
M40. 1-150 mg/mL EGFR/LGR5 BsAb; 3-12 mM histidine buffer system; a pH of 6.2-6.4; 100-320 mM sugar component; and 0.01-2 mg/mL surfactant.
M41. 15-25 mg/mL EGFR/LGR5 BsAb; 3-12 mM histidine buffer system; a pH of 6.2-6.4; 100-320 mM sugar component; and 0.01-2 mg/mL surfactant.
M42. 20 mg/mL EGFR/LGR5 BsAb; 3-12 mM histidine buffer system; a pH of 6.2-6.4; 100-320 mM sugar component; and 0.01-2 mg/mL surfactant.
M43. 1-150 mg/mL EGFR/LGR5 BsAb; 5 mM histidine buffer system; a pH of 6.2-6.4; 100-320 mM sugar component; and 0.01-2 mg/mL surfactant.
M44. 15-25 mg/mL EGFR/LGR5 BsAb; 5 mM histidine buffer system; a pH of 6.2-6.4; 100-320 mM sugar component; and 0.01-2 mg/mL surfactant.
M45. 20 mg/mL EGFR/LGR5 BsAb; 5 mM histidine buffer system; a pH of 6.2-6.4; 100-320 mM sugar component; and 0.01-2 mg/mL surfactant.
M46. 1-150 mg/mL EGFR/LGR5 BsAb; 10 mM histidine buffer system; a pH of 6.2-6.4; 100-320 mM sugar component; and 0.01-2 mg/mL surfactant.
M47. 15-25 mg/mL EGFR/LGR5 BsAb; 10 mM histidine buffer system; a pH of 6.2-6.4; 100-320 mM sugar component; and 0.01-2 mg/mL surfactant.
M48. 20 mg/mL EGFR/LGR5 BsAb; 10 mM histidine buffer system; a pH of 6.2-6.4; 100-320 mM sugar component; and 0.01-2 mg/mL surfactant.
M49. 1-150 mg/mL EGFR/LGR5 BsAb; 3-12 mM citrate buffer system; a pH of 6.2-7.4; 100-320 mM sugar component; and 0.01-2 mg/mL surfactant.
M50. 15-25 mg/mL EGFR/LGR5 BsAb; 3-12 mM citrate buffer system; a pH of 6.2-7.4; 100-320 mM sugar component; and 0.01-2 mg/mL surfactant.
M51. 20 mg/mL EGFR/LGR5 BsAb; 3-12 mM citrate buffer system; a pH of 6.2-7.4; 100-320 mM sugar component; and 0.01-2 mg/mL surfactant.
M52. 1-150 mg/mL EGFR/LGR5 BsAb; 10 mM citrate buffer system; a pH of 6.2-7.4; 100-320 mM sugar component; and 0.01-2 mg/mL surfactant.
M53. 15-25 mg/mL EGFR/LGR5 BsAb; 10 mM citrate buffer system; a pH of 6.2-7.4; 100-320 mM sugar component; and 0.01-2 mg/mL surfactant.
M54. 20 mg/mL EGFR/LGR5 BsAb; 10 mM citrate buffer system; a pH of 6.2-7.4; 100-320 mM sugar component; and 0.01-2 mg/mL surfactant.
M55. 1-150 mg/mL EGFR/LGR5 BsAb; 2-70 mM buffer system; a pH of 6.9-7.1; 100-320 mM sugar component; and 0.01-2 mg/mL surfactant.
M56. 15-25 mg/mL EGFR/LGR5 BsAb; 2-70 mM buffer system; a pH of 6.9-7.1; 100-320 mM sugar component; and 0.01-2 mg/mL surfactant.
M57. 20 mg/mL EGFR/LGR5 BsAb; 2-70 mM buffer system; a pH of 6.9-7.1; 100-320 mM sugar component; and 0.01-2 mg/mL surfactant.
M58. 1-150 mg/mL EGFR/LGR5 BsAb; 3-12 mM histidine buffer system; a pH of 6.9-7.1; 100-320 mM sugar component; and 0.01-2 mg/mL surfactant.
M59. 15-25 mg/mL EGFR/LGR5 BsAb; 3-12 mM histidine buffer system; a pH of 6.9-7.1; 100-320 mM sugar component; and 0.01-2 mg/mL surfactant.
M60. 20 mg/mL EGFR/LGR5 BsAb; 3-12 mM histidine buffer system; a pH of 6.9-7.1; 100-320 mM sugar component; and 0.01-2 mg/mL surfactant.
M61. 1-150 mg/mL EGFR/LGR5 BsAb; 5 mM histidine buffer system; a pH of 6.9-7.1; 100-320 mM sugar component; and 0.01-2 mg/mL surfactant.
M62. 15-25 mg/mL EGFR/LGR5 BsAb; 5 mM histidine buffer system; a pH of 6.9-7.1; 100-320 mM sugar component; and 0.01-2 mg/mL surfactant.
M63. 20 mg/mL EGFR/LGR5 BsAb; 5 mM histidine buffer system; a pH of 6.9-7.1; 100-320 mM sugar component; and 0.01-2 mg/mL surfactant.
M64. 1-150 mg/mL EGFR/LGR5 BsAb; 10 mM histidine buffer system; a pH of 6.9-7.1; 100-320 mM sugar component; and 0.01-2 mg/mL surfactant.
M65. 15-25 mg/mL EGFR/LGR5 BsAb; 10 mM histidine buffer system; a pH of 6.9-7.1; 100-320 mM sugar component; and 0.01-2 mg/mL surfactant.
M66. 20 mg/mL EGFR/LGR5 BsAb; 10 mM histidine buffer system; a pH of 6.9-7.1; 100-320 mM sugar component; and 0.01-2 mg/mL surfactant.
M67. 1-150 mg/mL EGFR/LGR5 BsAb; 3-12 mM citrate buffer system; a pH of 6.9-7.1; 100-320 mM sugar component; and 0.01-2 mg/mL surfactant.
M68. 15-25 mg/mL EGFR/LGR5 BsAb; 3-12 mM citrate buffer system; a pH of 6.9-7.1; 100-320 mM sugar component; and 0.01-2 mg/mL surfactant.
M69. 20 mg/mL EGFR/LGR5 BsAb; 3-12 mM citrate buffer system; a pH of 6.9-7.1; 100-320 mM sugar component; and 0.01-2 mg/mL surfactant.
M70. 1-150 mg/mL EGFR/LGR5 BsAb; 10 mM citrate buffer system; a pH of 6.9-7.1; 100-320 mM sugar component; and 0.01-2 mg/mL surfactant.
M71. 15-25 mg/mL EGFR/LGR5 BsAb; 10 mM citrate buffer system; a pH of 6.9-7.1; 100-320 mM sugar component; and 0.01-2 mg/mL surfactant.
M72. 20 mg/mL EGFR/LGR5 BsAb; 10 mM citrate buffer system; a pH of 6.9-7.1; 100-320 mM sugar component; and 0.01-2 mg/mL surfactant.
M73. 1-150 mg/mL EGFR/LGR5 BsAb; 2-70 mM buffer system; a pH of 5.4-7.2; 150-350 mM sucrose; and 0.01-2 mg/mL surfactant.
M74. 15-25 mg/mL EGFR/LGR5 BsAb; 2-70 mM buffer system; a pH of 5.4-7.2; 150-350 mM sucrose; and 0.01-2 mg/mL surfactant.
M75. 20 mg/mL EGFR/LGR5 BsAb; 2-70 mM buffer system; a pH of 5.4-7.2; 150-350 mM sucrose; and 0.01-2 mg/mL surfactant.
M76. 1-150 mg/mL EGFR/LGR5 BsAb; 3-12 mM histidine buffer system; a pH of 5.4-7.2; 150-350 mM sucrose; and 0.01-2 mg/mL surfactant.
M77. 15-25 mg/mL EGFR/LGR5 BsAb; 3-12 mM histidine buffer system; a pH of 5.4-7.2; 150-350 mM sucrose; and 0.01-2 mg/mL surfactant.
M78. 20 mg/mL EGFR/LGR5 BsAb; 3-12 mM histidine buffer system; a pH of 5.4-7.2; 150-350 mM sucrose; and 0.01-2 mg/mL surfactant.
M79. 1-150 mg/mL EGFR/LGR5 BsAb; 5 mM histidine buffer system; a pH of 5.4-7.2; 150-350 mM sucrose; and 0.01-2 mg/mL surfactant.
M80. 15-25 mg/mL EGFR/LGR5 BsAb; 5 mM histidine buffer system; a pH of 5.4-7.2; 150-350 mM sucrose; and 0.01-2 mg/mL surfactant.
M81. 20 mg/mL EGFR/LGR5 BsAb; 5 mM histidine buffer system; a pH of 5.4-7.2; 150-350 mM sucrose; and 0.01-2 mg/mL surfactant.
M82. 1-150 mg/mL EGFR/LGR5 BsAb; 10 mM histidine buffer system; a pH of 5.4-7.2; 150-350 mM sucrose; and 0.01-2 mg/mL surfactant.
M83. 15-25 mg/mL EGFR/LGR5 BsAb; 10 mM histidine buffer system; a pH of 5.4-7.2; 150-350 mM sucrose; and 0.01-2 mg/mL surfactant.
M84. 20 mg/mL EGFR/LGR5 BsAb; 10 mM histidine buffer system; a pH of 5.4-7.2; 150-350 mM sucrose; and 0.01-2 mg/mL surfactant.
M85. 1-150 mg/mL EGFR/LGR5 BsAb; 3-12 mM citrate buffer system; a pH of 5.8-7.8; 150-350 mM sucrose; and 0.01-2 mg/mL surfactant.
M86. 15-25 mg/mL EGFR/LGR5 BsAb; 3-12 mM citrate buffer system; a pH of 5.8-7.8; 150-350 mM sucrose; and 0.01-2 mg/mL surfactant.
M87. 20 mg/mL EGFR/LGR5 BsAb; 3-12 mM citrate buffer system; a pH of 5.8-7.8; 150-350 mM sucrose; and 0.01-2 mg/mL surfactant.
M88. 1-150 mg/mL EGFR/LGR5 BsAb; 10 mM citrate buffer system; a pH of 5.8-7.8; 150-350 mM sucrose; and 0.01-2 mg/mL surfactant.
M89. 15-25 mg/mL EGFR/LGR5 BsAb; 10 mM citrate buffer system; a pH of 5.8-7.8; 150-350 mM sucrose; and 0.01-2 mg/mL surfactant.
M90. 20 mg/mL EGFR/LGR5 BsAb; 10 mM citrate buffer system; a pH of 5.8-7.8; 150-350 mM sucrose; and 0.01-2 mg/mL surfactant.
M91. 1-150 mg/mL EGFR/LGR5 BsAb; 2-70 mM buffer system; a pH of 5.8-6.0; 150-350 mM sucrose; and 0.01-2 mg/mL surfactant.
M92. 15-25 mg/mL EGFR/LGR5 BsAb; 2-70 mM buffer system; a pH of 5.8-6.0; 150-350 mM sucrose; and 0.01-2 mg/mL surfactant.
M93. 20 mg/mL EGFR/LGR5 BsAb; 2-70 mM buffer system; a pH of 5.8-6.0; 150-350 mM sucrose; and 0.01-2 mg/mL surfactant.
M94. 1-150 mg/mL EGFR/LGR5 BsAb; 3-12 mM histidine buffer system; a pH of 5.8-6.0; 150-350 mM sucrose; and 0.01-2 mg/mL surfactant.
M95. 15-25 mg/mL EGFR/LGR5 BsAb; 3-12 mM histidine buffer system; a pH of 5.8-6.0; 150-350 mM sucrose; and 0.01-2 mg/mL surfactant.
M96. 20 mg/mL EGFR/LGR5 BsAb; 3-12 mM histidine buffer system; a pH of 5.8-6.0; 150-350 mM sucrose; and 0.01-2 mg/mL surfactant.
M97. 1-150 mg/mL EGFR/LGR5 BsAb; 5 mM histidine buffer system; a pH of 5.8-6.0; 150-350 mM sucrose; and 0.01-2 mg/mL surfactant.
M98. 15-25 mg/mL EGFR/LGR5 BsAb; 5 mM histidine buffer system; a pH of 5.8-6.0; 150-350 mM sucrose; and 0.01-2 mg/mL surfactant.
M99. 20 mg/mL EGFR/LGR5 BsAb; 5 mM histidine buffer system; a pH of 5.8-6.0; 150-350 mM sucrose; and 0.01-2 mg/mL surfactant.
M100. 1-150 mg/mL EGFR/LGR5 BsAb; 10 mM histidine buffer system; a pH of 5.8-6.0; 150-350 mM sucrose; and 0.01-2 mg/mL surfactant.
M101. 15-25 mg/mL EGFR/LGR5 BsAb; 10 mM histidine buffer system; a pH of 5.8-6.0; 150-350 mM sucrose; and 0.01-2 mg/mL surfactant.
M102. 20 mg/mL EGFR/LGR5 BsAb; 10 mM histidine buffer system; a pH of 5.8-6.0; 150-350 mM sucrose; and 0.01-2 mg/mL surfactant.
M103. 1-150 mg/mL EGFR/LGR5 BsAb; 3-12 mM citrate buffer system; a pH of 5.8-7; 150-350 mM sucrose; and 0.01-2 mg/mL surfactant.
M104. 15-25 mg/mL EGFR/LGR5 BsAb; 3-12 mM citrate buffer system; a pH of 5.8-7; 150-350 mM sucrose; and 0.01-2 mg/mL surfactant.
M105. 20 mg/mL EGFR/LGR5 BsAb; 3-12 mM citrate buffer system; a pH of 5.8-7; 150-350 mM sucrose; and 0.01-2 mg/mL surfactant.
M106. 1-150 mg/mL EGFR/LGR5 BsAb; 10 mM citrate buffer system; a pH of 5.8-7; 150-350 mM sucrose; and 0.01-2 mg/mL surfactant.
M107. 15-25 mg/mL EGFR/LGR5 BsAb; 10 mM citrate buffer system; a pH of 5.8-7; 150-350 mM sucrose; and 0.01-2 mg/mL surfactant.
M108. 20 mg/mL EGFR/LGR5 BsAb; 10 mM citrate buffer system; a pH of 5.8-7; 150-350 mM sucrose; and 0.01-2 mg/mL surfactant.
M109. 1-150 mg/mL EGFR/LGR5 BsAb; 2-70 mM buffer system; a pH of 6.2-6.4; 150-350 mM sucrose; and 0.01-2 mg/mL surfactant.
M110. 15-25 mg/mL EGFR/LGR5 BsAb; 2-70 mM buffer system; a pH of 6.2-6.4; 150-350 mM sucrose; and 0.01-2 mg/mL surfactant.
M111. 20 mg/mL EGFR/LGR5 BsAb; 2-70 mM buffer system; a pH of 6.2-6.4; 150-350 mM sucrose; and 0.01-2 mg/mL surfactant.
M112. 1-150 mg/mL EGFR/LGR5 BsAb; 3-12 mM histidine buffer system; a pH of 6.2-6.4; 150-350 mM sucrose; and 0.01-2 mg/mL surfactant.
M113. 15-25 mg/mL EGFR/LGR5 BsAb; 3-12 mM histidine buffer system; a pH of 6.2-6.4; 150-350 mM sucrose; and 0.01-2 mg/mL surfactant.
M114. 20 mg/mL EGFR/LGR5 BsAb; 3-12 mM histidine buffer system; a pH of 6.2-6.4; 150-350 mM sucrose; and 0.01-2 mg/mL surfactant.
M115. 1-150 mg/mL EGFR/LGR5 BsAb; 5 mM histidine buffer system; a pH of 6.2-6.4; 150-350 mM sucrose; and 0.01-2 mg/mL surfactant.
M116. 15-25 mg/mL EGFR/LGR5 BsAb; 5 mM histidine buffer system; a pH of 6.2-6.4; 150-350 mM sucrose; and 0.01-2 mg/mL surfactant.
M117. 20 mg/mL EGFR/LGR5 BsAb; 5 mM histidine buffer system; a pH of 6.2-6.4; 150-350 mM sucrose; and 0.01-2 mg/mL surfactant.
M118. 1-150 mg/mL EGFR/LGR5 BsAb; 10 mM histidine buffer system; a pH of 6.2-6.4; 150-350 mM sucrose; and 0.01-2 mg/mL surfactant.
M119. 15-25 mg/mL EGFR/LGR5 BsAb; 10 mM histidine buffer system; a pH of 6.2-6.4; 150-350 mM sucrose; and 0.01-2 mg/mL surfactant.
M120. 20 mg/mL EGFR/LGR5 BsAb; 10 mM histidine buffer system; a pH of 6.2-6.4; 150-350 mM sucrose; and 0.01-2 mg/mL surfactant.
M121. 1-150 mg/mL EGFR/LGR5 BsAb; 3-12 mM citrate buffer system; a pH of 6.2-7.4; 150-350 mM sucrose; and 0.01-2 mg/mL surfactant.
M122. 15-25 mg/mL EGFR/LGR5 BsAb; 3-12 mM citrate buffer system; a pH of 6.2-7.4; 150-350 mM sucrose; and 0.01-2 mg/mL surfactant.
M123. 20 mg/mL EGFR/LGR5 BsAb; 3-12 mM citrate buffer system; a pH of 6.2-7.4; 150-350 mM sucrose; and 0.01-2 mg/mL surfactant.
M124. 1-150 mg/mL EGFR/LGR5 BsAb; 10 mM citrate buffer system; a pH of 6.2-7.4; 150-350 mM sucrose; and 0.01-2 mg/mL surfactant.
M125. 15-25 mg/mL EGFR/LGR5 BsAb; 10 mM citrate buffer system; a pH of 6.2-7.4; 150-350 mM sucrose; and 0.01-2 mg/mL surfactant.
M126. 20 mg/mL EGFR/LGR5 BsAb; 10 mM citrate buffer system; a pH of 6.2-7.4; 150-350 mM sucrose; and 0.01-2 mg/mL surfactant.
M127. 1-150 mg/mL EGFR/LGR5 BsAb; 2-70 mM buffer system; a pH of 6.9-7.1; 150-350 mM sucrose; and 0.01-2 mg/mL surfactant.
M128. 15-25 mg/mL EGFR/LGR5 BsAb; 2-70 mM buffer system; a pH of 6.9-7.1; 150-350 mM sucrose; and 0.01-2 mg/mL surfactant.
M129. 20 mg/mL EGFR/LGR5 BsAb; 2-70 mM buffer system; a pH of 6.9-7.1; 150-350 mM sucrose; and 0.01-2 mg/mL surfactant.
M130. 1-150 mg/mL EGFR/LGR5 BsAb; 3-12 mM histidine buffer system; a pH of 6.9-7.1; 150-350 mM sucrose; and 0.01-2 mg/mL surfactant.
M131. 15-25 mg/mL EGFR/LGR5 BsAb; 3-12 mM histidine buffer system; a pH of 6.9-7.1; 150-350 mM sucrose; and 0.01-2 mg/mL surfactant.
M132. 20 mg/mL EGFR/LGR5 BsAb; 3-12 mM histidine buffer system; a pH of 6.9-7.1; 150-350 mM sucrose; and 0.01-2 mg/mL surfactant.
M133. 1-150 mg/mL EGFR/LGR5 BsAb; 5 mM histidine buffer system; a pH of 6.9-7.1; 150-350 mM sucrose; and 0.01-2 mg/mL surfactant.
M134. 15-25 mg/mL EGFR/LGR5 BsAb; 5 mM histidine buffer system; a pH of 6.9-7.1; 150-350 mM sucrose; and 0.01-2 mg/mL surfactant.
M135. 20 mg/mL EGFR/LGR5 BsAb; 5 mM histidine buffer system; a pH of 6.9-7.1; 150-350 mM sucrose; and 0.01-2 mg/mL surfactant.
M136. 1-150 mg/mL EGFR/LGR5 BsAb; 10 mM histidine buffer system; a pH of 6.9-7.1; 150-350 mM sucrose; and 0.01-2 mg/mL surfactant.
M137. 15-25 mg/mL EGFR/LGR5 BsAb; 10 mM histidine buffer system; a pH of 6.9-7.1; 150-350 mM sucrose; and 0.01-2 mg/mL surfactant.
M138. 20 mg/mL EGFR/LGR5 BsAb; 10 mM histidine buffer system; a pH of 6.9-7.1; 150-350 mM sucrose; and 0.01-2 mg/mL surfactant.
M139. 1-150 mg/mL EGFR/LGR5 BsAb; 3-12 mM citrate buffer system; a pH of 6.9-7.1; 150-350 mM sucrose; and 0.01-2 mg/mL surfactant.
M140. 15-25 mg/mL EGFR/LGR5 BsAb; 3-12 mM citrate buffer system; a pH of 6.9-7.1; 150-350 mM sucrose; and 0.01-2 mg/mL surfactant.
M141. 20 mg/mL EGFR/LGR5 BsAb; 3-12 mM citrate buffer system; a pH of 6.9-7.1; 150-350 mM sucrose; and 0.01-2 mg/mL surfactant.
M142. 1-150 mg/mL EGFR/LGR5 BsAb; 10 mM citrate buffer system; a pH of 6.9-7.1; 150-350 mM sucrose; and 0.01-2 mg/mL surfactant.
M143. 15-25 mg/mL EGFR/LGR5 BsAb; 10 mM citrate buffer system; a pH of 6.9-7.1; 150-350 mM sucrose; and 0.01-2 mg/mL surfactant.
M144. 20 mg/mL EGFR/LGR5 BsAb; 10 mM citrate buffer system; a pH of 6.9-7.1; 150-350 mM sucrose; and 0.01-2 mg/mL surfactant.
M145. 1-150 mg/mL EGFR/LGR5 BsAb; 2-70 mM buffer system; a pH of 5.4-7.2; 275-295 mM sucrose; and 0.01-2 mg/mL surfactant.
M146. 15-25 mg/mL EGFR/LGR5 BsAb; 2-70 mM buffer system; a pH of 5.4-7.2; 275-295 mM sucrose; and 0.01-2 mg/mL surfactant.
M147. 20 mg/mL EGFR/LGR5 BsAb; 2-70 mM buffer system; a pH of 5.4-7.2; 275-295 mM sucrose; and 0.01-2 mg/mL surfactant.
M148. 1-150 mg/mL EGFR/LGR5 BsAb; 3-12 mM histidine buffer system; a pH of 5.4-7.2; 275-295 mM sucrose; and 0.01-2 mg/mL surfactant.
M149. 15-25 mg/mL EGFR/LGR5 BsAb; 3-12 mM histidine buffer system; a pH of 5.4-7.2; 275-295 mM sucrose; and 0.01-2 mg/mL surfactant.
M150. 20 mg/mL EGFR/LGR5 BsAb; 3-12 mM histidine buffer system; a pH of 5.4-7.2; 275-295 mM sucrose; and 0.01-2 mg/mL surfactant.
M151. 1-150 mg/mL EGFR/LGR5 BsAb; 5 mM histidine buffer system; a pH of 5.4-7.2; 275-295 mM sucrose; and 0.01-2 mg/mL surfactant.
M152. 15-25 mg/mL EGFR/LGR5 BsAb; 5 mM histidine buffer system; a pH of 5.4-7.2; 275-295 mM sucrose; and 0.01-2 mg/mL surfactant.
M153. 20 mg/mL EGFR/LGR5 BsAb; 5 mM histidine buffer system; a pH of 5.4-7.2; 275-295 mM sucrose; and 0.01-2 mg/mL surfactant.
M154. 1-150 mg/mL EGFR/LGR5 BsAb; 10 mM histidine buffer system; a pH of 5.4-7.2; 275-295 mM sucrose; and 0.01-2 mg/mL surfactant.
M155. 15-25 mg/mL EGFR/LGR5 BsAb; 10 mM histidine buffer system; a pH of 5.4-7.2; 275-295 mM sucrose; and 0.01-2 mg/mL surfactant.
M156. 20 mg/mL EGFR/LGR5 BsAb; 10 mM histidine buffer system; a pH of 5.4-7.2; 275-295 mM sucrose; and 0.01-2 mg/mL surfactant.
M157. 1-150 mg/mL EGFR/LGR5 BsAb; 3-12 mM citrate buffer system; a pH of 5.8-7.8; 275-295 mM sucrose; and 0.01-2 mg/mL surfactant.
M158. 15-25 mg/mL EGFR/LGR5 BsAb; 3-12 mM citrate buffer system; a pH of 5.8-7.8; 275-295 mM sucrose; and 0.01-2 mg/mL surfactant.
M159. 20 mg/mL EGFR/LGR5 BsAb; 3-12 mM citrate buffer system; a pH of 5.8-7.8; 275-295 mM sucrose; and 0.01-2 mg/mL surfactant.
M160. 1-150 mg/mL EGFR/LGR5 BsAb; 10 mM citrate buffer system; a pH of 5.8-7.8; 275-295 mM sucrose; and 0.01-2 mg/mL surfactant.
M161. 15-25 mg/mL EGFR/LGR5 BsAb; 10 mM citrate buffer system; a pH of 5.8-7.8; 275-295 mM sucrose; and 0.01-2 mg/mL surfactant.
M162. 20 mg/mL EGFR/LGR5 BsAb; 10 mM citrate buffer system; a pH of 5.8-7.8; 275-295 mM sucrose; and 0.01-2 mg/mL surfactant.
M163. 1-150 mg/mL EGFR/LGR5 BsAb; 2-70 mM buffer system; a pH of 5.8-6.0; 275-295 mM sucrose; and 0.01-2 mg/mL surfactant.
M164. 15-25 mg/mL EGFR/LGR5 BsAb; 2-70 mM buffer system; a pH of 5.8-6.0; 275-295 mM sucrose; and 0.01-2 mg/mL surfactant.
M165. 20 mg/mL EGFR/LGR5 BsAb; 2-70 mM buffer system; a pH of 5.8-6.0; 275-295 mM sucrose; and 0.01-2 mg/mL surfactant.
M166. 1-150 mg/mL EGFR/LGR5 BsAb; 3-12 mM histidine buffer system; a pH of 5.8-6.0; 275-295 mM sucrose; and 0.01-2 mg/mL surfactant.
M167. 15-25 mg/mL EGFR/LGR5 BsAb; 3-12 mM histidine buffer system; a pH of 5.8-6.0; 275-295 mM sucrose; and 0.01-2 mg/mL surfactant.
M168. 20 mg/mL EGFR/LGR5 BsAb; 3-12 mM histidine buffer system; a pH of 5.8-6.0; 275-295 mM sucrose; and 0.01-2 mg/mL surfactant.
M169. 1-150 mg/mL EGFR/LGR5 BsAb; 5 mM histidine buffer system; a pH of 5.8-6.0; 275-295 mM sucrose; and 0.01-2 mg/mL surfactant.
M170. 15-25 mg/mL EGFR/LGR5 BsAb; 5 mM histidine buffer system; a pH of 5.8-6.0; 275-295 mM sucrose; and 0.01-2 mg/mL surfactant.
M171. 20 mg/mL EGFR/LGR5 BsAb; 5 mM histidine buffer system; a pH of 5.8-6.0; 275-295 mM sucrose; and 0.01-2 mg/mL surfactant.
M172. 1-150 mg/mL EGFR/LGR5 BsAb; 10 mM histidine buffer system; a pH of 5.8-6.0; 275-295 mM sucrose; and 0.01-2 mg/mL surfactant.
M173. 15-25 mg/mL EGFR/LGR5 BsAb; 10 mM histidine buffer system; a pH of 5.8-6.0; 275-295 mM sucrose; and 0.01-2 mg/mL surfactant.
M174. 20 mg/mL EGFR/LGR5 BsAb; 10 mM histidine buffer system; a pH of 5.8-6.0; 275-295 mM sucrose; and 0.01-2 mg/mL surfactant.
M175. 1-150 mg/mL EGFR/LGR5 BsAb; 3-12 mM citrate buffer system; a pH of 5.8-7; 275-295 mM sucrose; and 0.01-2 mg/mL surfactant.
M176. 15-25 mg/mL EGFR/LGR5 BsAb; 3-12 mM citrate buffer system; a pH of 5.8-7; 275-295 mM sucrose; and 0.01-2 mg/mL surfactant.
M177. 20 mg/mL EGFR/LGR5 BsAb; 3-12 mM citrate buffer system; a pH of 5.8-7; 275-295 mM sucrose; and 0.01-2 mg/mL surfactant.
M178. 1-150 mg/mL EGFR/LGR5 BsAb; 10 mM citrate buffer system; a pH of 5.8-7; 275-295 mM sucrose; and 0.01-2 mg/mL surfactant.
M179. 15-25 mg/mL EGFR/LGR5 BsAb; 10 mM citrate buffer system; a pH of 5.8-7; 275-295 mM sucrose; and 0.01-2 mg/mL surfactant.
M180. 20 mg/mL EGFR/LGR5 BsAb; 10 mM citrate buffer system; a pH of 5.8-7; 275-295 mM sucrose; and 0.01-2 mg/mL surfactant.
M181. 1-150 mg/mL EGFR/LGR5 BsAb; 2-70 mM buffer system; a pH of 6.2-6.4; 275-295 mM sucrose; and 0.01-2 mg/mL surfactant.
M182. 15-25 mg/mL EGFR/LGR5 BsAb; 2-70 mM buffer system; a pH of 6.2-6.4; 275-295 mM sucrose; and 0.01-2 mg/mL surfactant.
M183. 20 mg/mL EGFR/LGR5 BsAb; 2-70 mM buffer system; a pH of 6.2-6.4; 275-295 mM sucrose; and 0.01-2 mg/mL surfactant.
M184. 1-150 mg/mL EGFR/LGR5 BsAb; 3-12 mM histidine buffer system; a pH of 6.2-6.4; 275-295 mM sucrose; and 0.01-2 mg/mL surfactant.
M185. 15-25 mg/mL EGFR/LGR5 BsAb; 3-12 mM histidine buffer system; a pH of 6.2-6.4; 275-295 mM sucrose; and 0.01-2 mg/mL surfactant.
M186. 20 mg/mL EGFR/LGR5 BsAb; 3-12 mM histidine buffer system; a pH of 6.2-6.4; 275-295 mM sucrose; and 0.01-2 mg/mL surfactant.
M187. 1-150 mg/mL EGFR/LGR5 BsAb; 5 mM histidine buffer system; a pH of 6.2-6.4; 275-295 mM sucrose; and 0.01-2 mg/mL surfactant.
M188. 15-25 mg/mL EGFR/LGR5 BsAb; 5 mM histidine buffer system; a pH of 6.2-6.4; 275-295 mM sucrose; and 0.01-2 mg/mL surfactant.
M189. 20 mg/mL EGFR/LGR5 BsAb; 5 mM histidine buffer system; a pH of 6.2-6.4; 275-295 mM sucrose; and 0.01-2 mg/mL surfactant.
M190. 1-150 mg/mL EGFR/LGR5 BsAb; 10 mM histidine buffer system; a pH of 6.2-6.4; 275-295 mM sucrose; and 0.01-2 mg/mL surfactant.
M191. 15-25 mg/mL EGFR/LGR5 BsAb; 10 mM histidine buffer system; a pH of 6.2-6.4; 275-295 mM sucrose; and 0.01-2 mg/mL surfactant.
M192. 20 mg/mL EGFR/LGR5 BsAb; 10 mM histidine buffer system; a pH of 6.2-6.4; 275-295 mM sucrose; and 0.01-2 mg/mL surfactant.
M193. 1-150 mg/mL EGFR/LGR5 BsAb; 3-12 mM citrate buffer system; a pH of 6.2-7.4; 275-295 mM sucrose; and 0.01-2 mg/mL surfactant.
M194. 15-25 mg/mL EGFR/LGR5 BsAb; 3-12 mM citrate buffer system; a pH of 6.2-7.4; 275-295 mM sucrose; and 0.01-2 mg/mL surfactant.
M195. 20 mg/mL EGFR/LGR5 BsAb; 3-12 mM citrate buffer system; a pH of 6.2-7.4; 275-295 mM sucrose; and 0.01-2 mg/mL surfactant.
M196. 1-150 mg/mL EGFR/LGR5 BsAb; 10 mM citrate buffer system; a pH of 6.2-7.4; 275-295 mM sucrose; and 0.01-2 mg/mL surfactant.
M197. 15-25 mg/mL EGFR/LGR5 BsAb; 10 mM citrate buffer system; a pH of 6.2-7.4; 275-295 mM sucrose; and 0.01-2 mg/mL surfactant.
M198. 20 mg/mL EGFR/LGR5 BsAb; 10 mM citrate buffer system; a pH of 6.2-7.4; 275-295 mM sucrose; and 0.01-2 mg/mL surfactant.
M199. 1-150 mg/mL EGFR/LGR5 BsAb; 2-70 mM buffer system; a pH of 6.9-7.1; 275-295 mM sucrose; and 0.01-2 mg/mL surfactant.
M200. 15-25 mg/mL EGFR/LGR5 BsAb; 2-70 mM buffer system; a pH of 6.9-7.1; 275-295 mM sucrose; and 0.01-2 mg/mL surfactant.
M201. 20 mg/mL EGFR/LGR5 BsAb; 2-70 mM buffer system; a pH of 6.9-7.1; 275-295 mM sucrose; and 0.01-2 mg/mL surfactant.
M202. 1-150 mg/mL EGFR/LGR5 BsAb; 3-12 mM histidine buffer system; a pH of 6.9-7.1; 275-295 mM sucrose; and 0.01-2 mg/mL surfactant.
M203. 15-25 mg/mL EGFR/LGR5 BsAb; 3-12 mM histidine buffer system; a pH of 6.9-7.1; 275-295 mM sucrose; and 0.01-2 mg/mL surfactant.
M204. 20 mg/mL EGFR/LGR5 BsAb; 3-12 mM histidine buffer system; a pH of 6.9-7.1; 275-295 mM sucrose; and 0.01-2 mg/mL surfactant.
M205. 1-150 mg/mL EGFR/LGR5 BsAb; 5 mM histidine buffer system; a pH of 6.9-7.1; 275-295 mM sucrose; and 0.01-2 mg/mL surfactant.
M206. 15-25 mg/mL EGFR/LGR5 BsAb; 5 mM histidine buffer system; a pH of 6.9-7.1; 275-295 mM sucrose; and 0.01-2 mg/mL surfactant.
M207. 20 mg/mL EGFR/LGR5 BsAb; 5 mM histidine buffer system; a pH of 6.9-7.1; 275-295 mM sucrose; and 0.01-2 mg/mL surfactant.
M208. 1-150 mg/mL EGFR/LGR5 BsAb; 10 mM histidine buffer system; a pH of 6.9-7.1; 275-295 mM sucrose; and 0.01-2 mg/mL surfactant.
M209. 15-25 mg/mL EGFR/LGR5 BsAb; 10 mM histidine buffer system; a pH of 6.9-7.1; 275-295 mM sucrose; and 0.01-2 mg/mL surfactant.
M210. 20 mg/mL EGFR/LGR5 BsAb; 10 mM histidine buffer system; a pH of 6.9-7.1; 275-295 mM sucrose; and 0.01-2 mg/mL surfactant.
M211. 1-150 mg/mL EGFR/LGR5 BsAb; 3-12 mM citrate buffer system; a pH of 6.9-7.1; 275-295 mM sucrose; and 0.01-2 mg/mL surfactant.
M212. 15-25 mg/mL EGFR/LGR5 BsAb; 3-12 mM citrate buffer system; a pH of 6.9-7.1; 275-295 mM sucrose; and 0.01-2 mg/mL surfactant.
M213. 20 mg/mL EGFR/LGR5 BsAb; 3-12 mM citrate buffer system; a pH of 6.9-7.1; 275-295 mM sucrose; and 0.01-2 mg/mL surfactant.
M214. 1-150 mg/mL EGFR/LGR5 BsAb; 10 mM citrate buffer system; a pH of 6.9-7.1; 275-295 mM sucrose; and 0.01-2 mg/mL surfactant.
M215. 15-25 mg/mL EGFR/LGR5 BsAb; 10 mM citrate buffer system; a pH of 6.9-7.1; 275-295 mM sucrose; and 0.01-2 mg/mL surfactant.
M216. 20 mg/mL EGFR/LGR5 BsAb; 10 mM citrate buffer system; a pH of 6.9-7.1; 275-295 mM sucrose; and 0.01-2 mg/mL surfactant.
M217. 1-150 mg/mL EGFR/LGR5 BsAb; 2-70 mM buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M218. 15-25 mg/mL EGFR/LGR5 BsAb; 2-70 mM buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M219. 20 mg/mL EGFR/LGR5 BsAb; 2-70 mM buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M220. 1-150 mg/mL EGFR/LGR5 BsAb; 3-12 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M221. 15-25 mg/mL EGFR/LGR5 BsAb; 3-12 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M222. 20 mg/mL EGFR/LGR5 BsAb; 3-12 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M223. 1-150 mg/mL EGFR/LGR5 BsAb; 5 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M224. 15-25 mg/mL EGFR/LGR5 BsAb; 5 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M225. 20 mg/mL EGFR/LGR5 BsAb; 5 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M226. 1-150 mg/mL EGFR/LGR5 BsAb; 10 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M227. 15-25 mg/mL EGFR/LGR5 BsAb; 10 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M228. 20 mg/mL EGFR/LGR5 BsAb; 10 mM histidine buffer system; a pH of 5.4-7.2; 100-320 mM sugar component; and 0.01-1.5 mg/mL surfactant selected from the group consisting of polysorbate 20, polysorbate 80, poloxamer 188, and any combination thereof.
M229. 1-150 mg/mL EGFR/LGR5 BsAb; 3-12 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M230. 15-25 mg/mL EGFR/LGR5 BsAb; 3-12 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M231. 20 mg/mL EGFR/LGR5 BsAb; 3-12 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M232. 1-150 mg/mL EGFR/LGR5 BsAb; 10 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M233. 15-25 mg/mL EGFR/LGR5 BsAb; 10 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M234. 20 mg/mL EGFR/LGR5 BsAb; 10 mM citrate buffer system; a pH of 5.8-7.8; 100-320 mM sugar component; and 0.01-1.5 mg/mL surfactant selected from the group consisting of polysorbate 20, polysorbate 80, poloxamer 188, and any combination thereof.
M235. 1-150 mg/mL EGFR/LGR5 BsAb; 2-70 mM buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M236. 15-25 mg/mL EGFR/LGR5 BsAb; 2-70 mM buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M237. 20 mg/mL EGFR/LGR5 BsAb; 2-70 mM buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M238. 1-150 mg/mL EGFR/LGR5 BsAb; 3-12 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M239. 15-25 mg/mL EGFR/LGR5 BsAb; 3-12 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M240. 20 mg/mL EGFR/LGR5 BsAb; 3-12 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M241. 1-150 mg/mL EGFR/LGR5 BsAb; 5 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M242. 15-25 mg/mL EGFR/LGR5 BsAb; 5 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M243. 20 mg/mL EGFR/LGR5 BsAb; 5 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M244. 1-150 mg/mL EGFR/LGR5 BsAb; 10 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M245. 15-25 mg/mL EGFR/LGR5 BsAb; 10 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M246. 20 mg/mL EGFR/LGR5 BsAb; 10 mM histidine buffer system; a pH of 5.8-6.0; 100-320 mM sugar component; and 0.01-1.5 mg/mL surfactant selected from the group consisting of polysorbate 20, polysorbate 80, poloxamer 188, and any combination thereof.
M247. 1-150 mg/mL EGFR/LGR5 BsAb; 3-12 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M248. 15-25 mg/mL EGFR/LGR5 BsAb; 3-12 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M249. 20 mg/mL EGFR/LGR5 BsAb; 3-12 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M250. 1-150 mg/mL EGFR/LGR5 BsAb; 10 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M251. 15-25 mg/mL EGFR/LGR5 BsAb; 10 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M252. 20 mg/mL EGFR/LGR5 BsAb; 10 mM citrate buffer system; a pH of 5.8-7; 100-320 mM sugar component; and 0.01-1.5 mg/mL surfactant selected from the group consisting of polysorbate 20, polysorbate 80, poloxamer 188, and any combination thereof.
M253. 1-150 mg/mL EGFR/LGR5 BsAb; 2-70 mM buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M254. 15-25 mg/mL EGFR/LGR5 BsAb; 2-70 mM buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M255. 20 mg/mL EGFR/LGR5 BsAb; 2-70 mM buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M256. 1-150 mg/mL EGFR/LGR5 BsAb; 3-12 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M257. 15-25 mg/mL EGFR/LGR5 BsAb; 3-12 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M258. 20 mg/mL EGFR/LGR5 BsAb; 3-12 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M259. 1-150 mg/mL EGFR/LGR5 BsAb; 5 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M260. 15-25 mg/mL EGFR/LGR5 BsAb; 5 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M261. 20 mg/mL EGFR/LGR5 BsAb; 5 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M262. 1-150 mg/mL EGFR/LGR5 BsAb; 10 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M263. 15-25 mg/mL EGFR/LGR5 BsAb; 10 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M264. 20 mg/mL EGFR/LGR5 BsAb; 10 mM histidine buffer system; a pH of 6.2-6.4; 100-320 mM sugar component; and 0.01-1.5 mg/mL surfactant selected from the group consisting of polysorbate 20, polysorbate 80, poloxamer 188, and any combination thereof.
M265. 1-150 mg/mL EGFR/LGR5 BsAb; 3-12 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M266. 15-25 mg/mL EGFR/LGR5 BsAb; 3-12 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M267. 20 mg/mL EGFR/LGR5 BsAb; 3-12 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M268. 1-150 mg/mL EGFR/LGR5 BsAb; 10 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M269. 15-25 mg/mL EGFR/LGR5 BsAb; 10 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M270. 20 mg/mL EGFR/LGR5 BsAb; 10 mM citrate buffer system; a pH of 6.2-7.4; 100-320 mM sugar component; and 0.01-1.5 mg/mL surfactant selected from the group consisting of polysorbate 20, polysorbate 80, poloxamer 188, and any combination thereof.
M271. 1-150 mg/mL EGFR/LGR5 BsAb; 2-70 mM buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M272. 15-25 mg/mL EGFR/LGR5 BsAb; 2-70 mM buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M273. 20 mg/mL EGFR/LGR5 BsAb; 2-70 mM buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M274. 1-150 mg/mL EGFR/LGR5 BsAb; 3-12 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M275. 15-25 mg/mL EGFR/LGR5 BsAb; 3-12 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M276. 20 mg/mL EGFR/LGR5 BsAb; 3-12 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M277. 1-150 mg/mL EGFR/LGR5 BsAb; 5 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M278. 15-25 mg/mL EGFR/LGR5 BsAb; 5 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M279. 20 mg/mL EGFR/LGR5 BsAb; 5 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M280. 1-150 mg/mL EGFR/LGR5 BsAb; 10 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M281. 15-25 mg/mL EGFR/LGR5 BsAb; 10 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M282. 20 mg/mL EGFR/LGR5 BsAb; 10 mM histidine buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M283. 1-150 mg/mL EGFR/LGR5 BsAb; 3-12 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M284. 15-25 mg/mL EGFR/LGR5 BsAb; 3-12 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M285. 20 mg/mL EGFR/LGR5 BsAb; 3-12 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M286. 1-150 mg/mL EGFR/LGR5 BsAb; 10 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M287. 15-25 mg/mL EGFR/LGR5 BsAb; 10 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M288. 20 mg/mL EGFR/LGR5 BsAb; 10 mM citrate buffer system; a pH of 6.9-7.1; 100-320 mM sugar component; and 0.01-1.5 mg/mL surfactant selected from the group consisting of polysorbate 20, polysorbate 80, poloxamer 188, and any combination thereof.
M289. 1-150 mg/mL EGFR/LGR5 BsAb; 2-70 mM buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M290. 15-25 mg/mL EGFR/LGR5 BsAb; 2-70 mM buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M291. 20 mg/mL EGFR/LGR5 BsAb; 2-70 mM buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M292. 1-150 mg/mL EGFR/LGR5 BsAb; 3-12 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M293. 15-25 mg/mL EGFR/LGR5 BsAb; 3-12 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M294. 20 mg/mL EGFR/LGR5 BsAb; 3-12 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M295. 1-150 mg/mL EGFR/LGR5 BsAb; 5 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M296. 15-25 mg/mL EGFR/LGR5 BsAb; 5 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M297. 20 mg/mL EGFR/LGR5 BsAb; 5 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M298. 1-150 mg/mL EGFR/LGR5 BsAb; 10 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M299. 15-25 mg/mL EGFR/LGR5 BsAb; 10 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M300. 20 mg/mL EGFR/LGR5 BsAb; 10 mM histidine buffer system; a pH of 5.4-7.2; 150-350 mM sucrose; and 0.01-1.5 mg/mL surfactant selected from the group consisting of polysorbate 20, polysorbate 80, poloxamer 188, and any combination thereof.
M301. 1-150 mg/mL EGFR/LGR5 BsAb; 3-12 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M302. 15-25 mg/mL EGFR/LGR5 BsAb; 3-12 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M303. 20 mg/mL EGFR/LGR5 BsAb; 3-12 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M304. 1-150 mg/mL EGFR/LGR5 BsAb; 10 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M305. 15-25 mg/mL EGFR/LGR5 BsAb; 10 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M306. 20 mg/mL EGFR/LGR5 BsAb; 10 mM citrate buffer system; a pH of 5.8-7.8; 150-350 mM sucrose; and 0.01-1.5 mg/mL surfactant selected from the group consisting of polysorbate 20, polysorbate 80, poloxamer 188, and any combination thereof.
M307. 1-150 mg/mL EGFR/LGR5 BsAb; 2-70 mM buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M308. 15-25 mg/mL EGFR/LGR5 BsAb; 2-70 mM buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M309. 20 mg/mL EGFR/LGR5 BsAb; 2-70 mM buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M310. 1-150 mg/mL EGFR/LGR5 BsAb; 3-12 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M311. 15-25 mg/mL EGFR/LGR5 BsAb; 3-12 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M312. 20 mg/mL EGFR/LGR5 BsAb; 3-12 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M313. 1-150 mg/mL EGFR/LGR5 BsAb; 5 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M314. 15-25 mg/mL EGFR/LGR5 BsAb; 5 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M315. 20 mg/mL EGFR/LGR5 BsAb; 5 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M316. 1-150 mg/mL EGFR/LGR5 BsAb; 10 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M317. 15-25 mg/mL EGFR/LGR5 BsAb; 10 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M318. 20 mg/mL EGFR/LGR5 BsAb; 10 mM histidine buffer system; a pH of 5.8-6.0; 150-350 mM sucrose; and 0.01-1.5 mg/mL surfactant selected from the group consisting of polysorbate 20, polysorbate 80, poloxamer 188, and any combination thereof.
M319. 1-150 mg/mL EGFR/LGR5 BsAb; 3-12 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M320. 15-25 mg/mL EGFR/LGR5 BsAb; 3-12 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M321. 20 mg/mL EGFR/LGR5 BsAb; 3-12 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M322. 1-150 mg/mL EGFR/LGR5 BsAb; 10 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M323. 15-25 mg/mL EGFR/LGR5 BsAb; 10 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M324. 20 mg/mL EGFR/LGR5 BsAb; 10 mM citrate buffer system; a pH of 5.8-7; 150-350 mM sucrose; and 0.01-1.5 mg/mL surfactant selected from the group consisting of polysorbate 20, polysorbate 80, poloxamer 188, and any combination thereof.
M325. 1-150 mg/mL EGFR/LGR5 BsAb; 2-70 mM buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M326. 15-25 mg/mL EGFR/LGR5 BsAb; 2-70 mM buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M327. 20 mg/mL EGFR/LGR5 BsAb; 2-70 mM buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M328. 1-150 mg/mL EGFR/LGR5 BsAb; 3-12 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M329. 15-25 mg/mL EGFR/LGR5 BsAb; 3-12 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M330. 20 mg/mL EGFR/LGR5 BsAb; 3-12 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M331. 1-150 mg/mL EGFR/LGR5 BsAb; 5 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M332. 15-25 mg/mL EGFR/LGR5 BsAb; 5 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M333. 20 mg/mL EGFR/LGR5 BsAb; 5 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M334. 1-150 mg/mL EGFR/LGR5 BsAb; 10 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M335. 15-25 mg/mL EGFR/LGR5 BsAb; 10 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M336. 20 mg/mL EGFR/LGR5 BsAb; 10 mM histidine buffer system; a pH of 6.2-6.4; 150-350 mM sucrose; and 0.01-1.5 mg/mL surfactant selected from the group consisting of polysorbate 20, polysorbate 80, poloxamer 188, and any combination thereof.
M337. 1-150 mg/mL EGFR/LGR5 BsAb; 3-12 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M338. 15-25 mg/mL EGFR/LGR5 BsAb; 3-12 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M339. 20 mg/mL EGFR/LGR5 BsAb; 3-12 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M340. 1-150 mg/mL EGFR/LGR5 BsAb; 10 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M341. 15-25 mg/mL EGFR/LGR5 BsAb; 10 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M342. 20 mg/mL EGFR/LGR5 BsAb; 10 mM citrate buffer system; a pH of 6.2-7.4; 150-350 mM sucrose; and 0.01-1.5 mg/mL surfactant selected from the group consisting of polysorbate 20, polysorbate 80, poloxamer 188, and any combination thereof.
M343. 1-150 mg/mL EGFR/LGR5 BsAb; 2-70 mM buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M344. 15-25 mg/mL EGFR/LGR5 BsAb; 2-70 mM buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M345. 20 mg/mL EGFR/LGR5 BsAb; 2-70 mM buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M346. 1-150 mg/mL EGFR/LGR5 BsAb; 3-12 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M347. 15-25 mg/mL EGFR/LGR5 BsAb; 3-12 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M348. 20 mg/mL EGFR/LGR5 BsAb; 3-12 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M349. 1-150 mg/mL EGFR/LGR5 BsAb; 5 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M350. 15-25 mg/mL EGFR/LGR5 BsAb; 5 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M351. 20 mg/mL EGFR/LGR5 BsAb; 5 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M352. 1-150 mg/mL EGFR/LGR5 BsAb; 10 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M353. 15-25 mg/mL EGFR/LGR5 BsAb; 10 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M354. 20 mg/mL EGFR/LGR5 BsAb; 10 mM histidine buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M355. 1-150 mg/mL EGFR/LGR5 BsAb; 3-12 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M356. 15-25 mg/mL EGFR/LGR5 BsAb; 3-12 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M357. 20 mg/mL EGFR/LGR5 BsAb; 3-12 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M358. 1-150 mg/mL EGFR/LGR5 BsAb; 10 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M359. 15-25 mg/mL EGFR/LGR5 BsAb; 10 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M360. 20 mg/mL EGFR/LGR5 BsAb; 10 mM citrate buffer system; a pH of 6.9-7.1; 150-350 mM sucrose; and 0.01-1.5 mg/mL surfactant selected from the group consisting of polysorbate 20, polysorbate 80, poloxamer 188, and any combination thereof.
M361. 1-150 mg/mL EGFR/LGR5 BsAb; 2-70 mM buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M362. 15-25 mg/mL EGFR/LGR5 BsAb; 2-70 mM buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M363. 20 mg/mL EGFR/LGR5 BsAb; 2-70 mM buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M364. 1-150 mg/mL EGFR/LGR5 BsAb; 3-12 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M365. 15-25 mg/mL EGFR/LGR5 BsAb; 3-12 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M366. 20 mg/mL EGFR/LGR5 BsAb; 3-12 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M367. 1-150 mg/mL EGFR/LGR5 BsAb; 5 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M368. 15-25 mg/mL EGFR/LGR5 BsAb; 5 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M369. 20 mg/mL EGFR/LGR5 BsAb; 5 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M370. 1-150 mg/mL EGFR/LGR5 BsAb; 10 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M371. 15-25 mg/mL EGFR/LGR5 BsAb; 10 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M372. 20 mg/mL EGFR/LGR5 BsAb; 10 mM histidine buffer system; a pH of 5.4-7.2; 275-295 mM sucrose; and 0.01-1.5 mg/mL surfactant selected from the group consisting of polysorbate 20, polysorbate 80, poloxamer 188, and any combination thereof.
M373. 1-150 mg/mL EGFR/LGR5 BsAb; 3-12 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M374. 15-25 mg/mL EGFR/LGR5 BsAb; 3-12 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M375. 20 mg/mL EGFR/LGR5 BsAb; 3-12 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M376. 1-150 mg/mL EGFR/LGR5 BsAb; 10 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M377. 15-25 mg/mL EGFR/LGR5 BsAb; 10 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M378. 20 mg/mL EGFR/LGR5 BsAb; 10 mM citrate buffer system; a pH of 5.8-7.8; 275-295 mM sucrose; and 0.01-1.5 mg/mL surfactant selected from the group consisting of polysorbate 20, polysorbate 80, poloxamer 188, and any combination thereof.
M379. 1-150 mg/mL EGFR/LGR5 BsAb; 2-70 mM buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M380. 15-25 mg/mL EGFR/LGR5 BsAb; 2-70 mM buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M381. 20 mg/mL EGFR/LGR5 BsAb; 2-70 mM buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M382. 1-150 mg/mL EGFR/LGR5 BsAb; 3-12 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M383. 15-25 mg/mL EGFR/LGR5 BsAb; 3-12 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M384. 20 mg/mL EGFR/LGR5 BsAb; 3-12 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M385. 1-150 mg/mL EGFR/LGR5 BsAb; 5 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M386. 15-25 mg/mL EGFR/LGR5 BsAb; 5 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M387. 20 mg/mL EGFR/LGR5 BsAb; 5 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M388. 1-150 mg/mL EGFR/LGR5 BsAb; 10 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M389. 15-25 mg/mL EGFR/LGR5 BsAb; 10 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M390. 20 mg/mL EGFR/LGR5 BsAb; 10 mM histidine buffer system; a pH of 5.8-6.0; 275-295 mM sucrose; and 0.01-1.5 mg/mL surfactant selected from the group consisting of polysorbate 20, polysorbate 80, poloxamer 188, and any combination thereof.
M391. 1-150 mg/mL EGFR/LGR5 BsAb; 3-12 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M392. 15-25 mg/mL EGFR/LGR5 BsAb; 3-12 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M393. 20 mg/mL EGFR/LGR5 BsAb; 3-12 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M394. 1-150 mg/mL EGFR/LGR5 BsAb; 10 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M395. 15-25 mg/mL EGFR/LGR5 BsAb; 10 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M396. 20 mg/mL EGFR/LGR5 BsAb; 10 mM citrate buffer system; a pH of 5.8-7; 275-295 mM sucrose; and 0.01-1.5 mg/mL surfactant selected from the group consisting of polysorbate 20, polysorbate 80, poloxamer 188, and any combination thereof.
M397. 1-150 mg/mL EGFR/LGR5 BsAb; 2-70 mM buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M398. 15-25 mg/mL EGFR/LGR5 BsAb; 2-70 mM buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M399. 20 mg/mL EGFR/LGR5 BsAb; 2-70 mM buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M400. 1-150 mg/mL EGFR/LGR5 BsAb; 3-12 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M401. 15-25 mg/mL EGFR/LGR5 BsAb; 3-12 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M402. 20 mg/mL EGFR/LGR5 BsAb; 3-12 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M403. 1-150 mg/mL EGFR/LGR5 BsAb; 5 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M404. 15-25 mg/mL EGFR/LGR5 BsAb; 5 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M405. 20 mg/mL EGFR/LGR5 BsAb; 5 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M406. 1-150 mg/mL EGFR/LGR5 BsAb; 10 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M407. 15-25 mg/mL EGFR/LGR5 BsAb; 10 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M408. 20 mg/mL EGFR/LGR5 BsAb; 10 mM histidine buffer system; a pH of 6.2-6.4; 275-295 mM sucrose; and 0.01-1.5 mg/mL surfactant selected from the group consisting of polysorbate 20, polysorbate 80, poloxamer 188, and any combination thereof.
M409. 1-150 mg/mL EGFR/LGR5 BsAb; 3-12 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M410. 15-25 mg/mL EGFR/LGR5 BsAb; 3-12 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M411. 20 mg/mL EGFR/LGR5 BsAb; 3-12 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M412. 1-150 mg/mL EGFR/LGR5 BsAb; 10 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M413. 15-25 mg/mL EGFR/LGR5 BsAb; 10 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M414. 20 mg/mL EGFR/LGR5 BsAb; 10 mM citrate buffer system; a pH of 6.2-7.4; 275-295 mM sucrose; and 0.01-1.5 mg/mL surfactant selected from the group consisting of polysorbate 20, polysorbate 80, poloxamer 188, and any combination thereof.
M415. 1-150 mg/mL EGFR/LGR5 BsAb; 2-70 mM buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M416. 15-25 mg/mL EGFR/LGR5 BsAb; 2-70 mM buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M417. 20 mg/mL EGFR/LGR5 BsAb; 2-70 mM buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M418. 1-150 mg/mL EGFR/LGR5 BsAb; 3-12 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M419. 15-25 mg/mL EGFR/LGR5 BsAb; 3-12 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M420. 20 mg/mL EGFR/LGR5 BsAb; 3-12 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M421. 1-150 mg/mL EGFR/LGR5 BsAb; 5 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M422. 15-25 mg/mL EGFR/LGR5 BsAb; 5 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M423. 20 mg/mL EGFR/LGR5 BsAb; 5 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M424. 1-150 mg/mL EGFR/LGR5 BsAb; 10 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M425. 15-25 mg/mL EGFR/LGR5 BsAb; 10 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M426. 20 mg/mL EGFR/LGR5 BsAb; 10 mM histidine buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M427. 1-150 mg/mL EGFR/LGR5 BsAb; 3-12 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M428. 15-25 mg/mL EGFR/LGR5 BsAb; 3-12 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M429. 20 mg/mL EGFR/LGR5 BsAb; 3-12 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M430. 1-150 mg/mL EGFR/LGR5 BsAb; 10 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M431. 15-25 mg/mL EGFR/LGR5 BsAb; 10 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M432. 20 mg/mL EGFR/LGR5 BsAb; 10 mM citrate buffer system; a pH of 6.9-7.1; 275-295 mM sucrose; and 0.01-1.5 mg/mL surfactant selected from the group consisting of polysorbate 20, polysorbate 80, poloxamer 188, and any combination thereof.
M433. 1-150 mg/mL EGFR/LGR5 BsAb; 2-70 mM buffer system; a pH of 5.4-7.2; 100-320 mM sugar component; and 0.1-0.3 mg/mL polysorbate 80.
M434. 15-25 mg/mL EGFR/LGR5 BsAb; 2-70 mM buffer system; a pH of 5.4-7.2; 100-320 mM sugar component; and 0.1-0.3 mg/mL polysorbate 80.
M435. 20 mg/mL EGFR/LGR5 BsAb; 2-70 mM buffer system; a pH of 5.4-7.2; 100-320 mM sugar component; and 0.1-0.3 mg/mL polysorbate 80.
M436. 1-150 mg/mL EGFR/LGR5 BsAb; 3-12 mM histidine buffer system; a pH of 5.4-7.2; 100-320 mM sugar component; and 0.1-0.3 mg/mL polysorbate 80.
M437. 15-25 mg/mL EGFR/LGR5 BsAb; 3-12 mM histidine buffer system; a pH of 5.4-7.2; 100-320 mM sugar component; and 0.1-0.3 mg/mL polysorbate 80.
M438. 20 mg/mL EGFR/LGR5 BsAb; 3-12 mM histidine buffer system; a pH of 5.4-7.2; 100-320 mM sugar component; and 0.1-0.3 mg/mL polysorbate 80.
M439. 1-150 mg/mL EGFR/LGR5 BsAb; 5 mM histidine buffer system; a pH of 5.4-7.2; 100-320 mM sugar component; and 0.1-0.3 mg/mL polysorbate 80.
M440. 15-25 mg/mL EGFR/LGR5 BsAb; 5 mM histidine buffer system; a pH of 5.4-7.2; 100-320 mM sugar component; and 0.1-0.3 mg/mL polysorbate 80.
M441. 20 mg/mL EGFR/LGR5 BsAb; 5 mM histidine buffer system; a pH of 5.4-7.2; 100-320 mM sugar component; and 0.1-0.3 mg/mL polysorbate 80.
M442. 1-150 mg/mL EGFR/LGR5 BsAb; 10 mM histidine buffer system; a pH of 5.4-7.2; 100-320 mM sugar component; and 0.1-0.3 mg/mL polysorbate 80.
M443. 15-25 mg/mL EGFR/LGR5 BsAb; 10 mM histidine buffer system; a pH of 5.4-7.2; 100-320 mM sugar component; and 0.1-0.3 mg/mL polysorbate 80.
M444. 20 mg/mL EGFR/LGR5 BsAb; 10 mM histidine buffer system; a pH of 5.4-7.2; 100-320 mM sugar component; and 0.1-0.3 mg/mL polysorbate 80.
M445. 1-150 mg/mL EGFR/LGR5 BsAb; 3-12 mM citrate buffer system; a pH of 5.8-7.8; 100-320 mM sugar component; and 0.1-0.3 mg/mL polysorbate 80.
M446. 15-25 mg/mL EGFR/LGR5 BsAb; 3-12 mM citrate buffer system; a pH of 5.8-7.8; 100-320 mM sugar component; and 0.1-0.3 mg/mL polysorbate 80.
M447. 20 mg/mL EGFR/LGR5 BsAb; 3-12 mM citrate buffer system; a pH of 5.8-7.8; 100-320 mM sugar component; and 0.1-0.3 mg/mL polysorbate 80.
M448. 1-150 mg/mL EGFR/LGR5 BsAb; 10 mM citrate buffer system; a pH of 5.8-7.8; 100-320 mM sugar component; and 0.1-0.3 mg/mL polysorbate 80.
M449. 15-25 mg/mL EGFR/LGR5 BsAb; 10 mM citrate buffer system; a pH of 5.8-7.8; 100-320 mM sugar component; and 0.1-0.3 mg/mL polysorbate 80.
M450. 20 mg/mL EGFR/LGR5 BsAb; 10 mM citrate buffer system; a pH of 5.8-7.8; 100-320 mM sugar component; and 0.1-0.3 mg/mL polysorbate 80.
M451. 1-150 mg/mL EGFR/LGR5 BsAb; 2-70 mM buffer system; a pH of 5.8-6.0; 100-320 mM sugar component; and 0.1-0.3 mg/mL polysorbate 80.
M452. 15-25 mg/mL EGFR/LGR5 BsAb; 2-70 mM buffer system; a pH of 5.8-6.0; 100-320 mM sugar component; and 0.1-0.3 mg/mL polysorbate 80.
M453. 20 mg/mL EGFR/LGR5 BsAb; 2-70 mM buffer system; a pH of 5.8-6.0; 100-320 mM sugar component; and 0.1-0.3 mg/mL polysorbate 80.
M454. 1-150 mg/mL EGFR/LGR5 BsAb; 3-12 mM histidine buffer system; a pH of 5.8-6.0; 100-320 mM sugar component; and 0.1-0.3 mg/mL polysorbate 80.
M455. 15-25 mg/mL EGFR/LGR5 BsAb; 3-12 mM histidine buffer system; a pH of 5.8-6.0; 100-320 mM sugar component; and 0.1-0.3 mg/mL polysorbate 80.
M456. 20 mg/mL EGFR/LGR5 BsAb; 3-12 mM histidine buffer system; a pH of 5.8-6.0; 100-320 mM sugar component; and 0.1-0.3 mg/mL polysorbate 80.
M457. 1-150 mg/mL EGFR/LGR5 BsAb; 5 mM histidine buffer system; a pH of 5.8-6.0; 100-320 mM sugar component; and 0.1-0.3 mg/mL polysorbate 80.
M458. 15-25 mg/mL EGFR/LGR5 BsAb; 5 mM histidine buffer system; a pH of 5.8-6.0; 100-320 mM sugar component; and 0.1-0.3 mg/mL polysorbate 80.
M459. 20 mg/mL EGFR/LGR5 BsAb; 5 mM histidine buffer system; a pH of 5.8-6.0; 100-320 mM sugar component; and 0.1-0.3 mg/mL polysorbate 80.
M460. 1-150 mg/mL EGFR/LGR5 BsAb; 10 mM histidine buffer system; a pH of 5.8-6.0; 100-320 mM sugar component; and 0.1-0.3 mg/mL polysorbate 80.
M461. 15-25 mg/mL EGFR/LGR5 BsAb; 10 mM histidine buffer system; a pH of 5.8-6.0; 100-320 mM sugar component; and 0.1-0.3 mg/mL polysorbate 80.
M462. 20 mg/mL EGFR/LGR5 BsAb; 10 mM histidine buffer system; a pH of 5.8-6.0; 100-320 mM sugar component; and 0.1-0.3 mg/mL polysorbate 80.
M463. 1-150 mg/mL EGFR/LGR5 BsAb; 3-12 mM citrate buffer system; a pH of 5.8-7; 100-320 mM sugar component; and 0.1-0.3 mg/mL polysorbate 80.
M464. 15-25 mg/mL EGFR/LGR5 BsAb; 3-12 mM citrate buffer system; a pH of 5.8-7; 100-320 mM sugar component; and 0.1-0.3 mg/mL polysorbate 80.
M465. 20 mg/mL EGFR/LGR5 BsAb; 3-12 mM citrate buffer system; a pH of 5.8-7; 100-320 mM sugar component; and 0.1-0.3 mg/mL polysorbate 80.
M466. 1-150 mg/mL EGFR/LGR5 BsAb; 10 mM citrate buffer system; a pH of 5.8-7; 100-320 mM sugar component; and 0.1-0.3 mg/mL polysorbate 80.
M467. 15-25 mg/mL EGFR/LGR5 BsAb; 10 mM citrate buffer system; a pH of 5.8-7; 100-320 mM sugar component; and 0.1-0.3 mg/mL polysorbate 80.
M468. 20 mg/mL EGFR/LGR5 BsAb; 10 mM citrate buffer system; a pH of 5.8-7; 100-320 mM sugar component; and 0.1-0.3 mg/mL polysorbate 80.
M469. 1-150 mg/mL EGFR/LGR5 BsAb; 2-70 mM buffer system; a pH of 6.2-6.4; 100-320 mM sugar component; and 0.1-0.3 mg/mL polysorbate 80.
M470. 15-25 mg/mL EGFR/LGR5 BsAb; 2-70 mM buffer system; a pH of 6.2-6.4; 100-320 mM sugar component; and 0.1-0.3 mg/mL polysorbate 80.
M471. 20 mg/mL EGFR/LGR5 BsAb; 2-70 mM buffer system; a pH of 6.2-6.4; 100-320 mM sugar component; and 0.1-0.3 mg/mL polysorbate 80.
M472. 1-150 mg/mL EGFR/LGR5 BsAb; 3-12 mM histidine buffer system; a pH of 6.2-6.4; 100-320 mM sugar component; and 0.1-0.3 mg/mL polysorbate 80.
M473. 15-25 mg/mL EGFR/LGR5 BsAb; 3-12 mM histidine buffer system; a pH of 6.2-6.4; 100-320 mM sugar component; and 0.1-0.3 mg/mL polysorbate 80.
M474. 20 mg/mL EGFR/LGR5 BsAb; 3-12 mM histidine buffer system; a pH of 6.2-6.4; 100-320 mM sugar component; and 0.1-0.3 mg/mL polysorbate 80.
M475. 1-150 mg/mL EGFR/LGR5 BsAb; 5 mM histidine buffer system; a pH of 6.2-6.4; 100-320 mM sugar component; and 0.1-0.3 mg/mL polysorbate 80.
M476. 15-25 mg/mL EGFR/LGR5 BsAb; 5 mM histidine buffer system; a pH of 6.2-6.4; 100-320 mM sugar component; and 0.1-0.3 mg/mL polysorbate 80.
M477. 20 mg/mL EGFR/LGR5 BsAb; 5 mM histidine buffer system; a pH of 6.2-6.4; 100-320 mM sugar component; and 0.1-0.3 mg/mL polysorbate 80.
M478. 1-150 mg/mL EGFR/LGR5 BsAb; 10 mM histidine buffer system; a pH of 6.2-6.4; 100-320 mM sugar component; and 0.1-0.3 mg/mL polysorbate 80.
M479. 15-25 mg/mL EGFR/LGR5 BsAb; 10 mM histidine buffer system; a pH of 6.2-6.4; 100-320 mM sugar component; and 0.1-0.3 mg/mL polysorbate 80.
M480. 20 mg/mL EGFR/LGR5 BsAb; 10 mM histidine buffer system; a pH of 6.2-6.4; 100-320 mM sugar component; and 0.1-0.3 mg/mL polysorbate 80.
M481. 1-150 mg/mL EGFR/LGR5 BsAb; 3-12 mM citrate buffer system; a pH of 6.2-7.4; 100-320 mM sugar component; and 0.1-0.3 mg/mL polysorbate 80.
M482. 15-25 mg/mL EGFR/LGR5 BsAb; 3-12 mM citrate buffer system; a pH of 6.2-7.4; 100-320 mM sugar component; and 0.1-0.3 mg/mL polysorbate 80.
M483. 20 mg/mL EGFR/LGR5 BsAb; 3-12 mM citrate buffer system; a pH of 6.2-7.4; 100-320 mM sugar component; and 0.1-0.3 mg/mL polysorbate 80.
M484. 1-150 mg/mL EGFR/LGR5 BsAb; 10 mM citrate buffer system; a pH of 6.2-7.4; 100-320 mM sugar component; and 0.1-0.3 mg/mL polysorbate 80.
M485. 15-25 mg/mL EGFR/LGR5 BsAb; 10 mM citrate buffer system; a pH of 6.2-7.4; 100-320 mM sugar component; and 0.1-0.3 mg/mL polysorbate 80.
M486. 20 mg/mL EGFR/LGR5 BsAb; 10 mM citrate buffer system; a pH of 6.2-7.4; 100-320 mM sugar component; and 0.1-0.3 mg/mL polysorbate 80.
M487. 1-150 mg/mL EGFR/LGR5 BsAb; 2-70 mM buffer system; a pH of 6.9-7.1; 100-320 mM sugar component; and 0.1-0.3 mg/mL polysorbate 80.
M488. 15-25 mg/mL EGFR/LGR5 BsAb; 2-70 mM buffer system; a pH of 6.9-7.1; 100-320 mM sugar component; and 0.1-0.3 mg/mL polysorbate 80.
M489. 20 mg/mL EGFR/LGR5 BsAb; 2-70 mM buffer system; a pH of 6.9-7.1; 100-320 mM sugar component; and 0.1-0.3 mg/mL polysorbate 80.
M490. 1-150 mg/mL EGFR/LGR5 BsAb; 3-12 mM histidine buffer system; a pH of 6.9-7.1; 100-320 mM sugar component; and 0.1-0.3 mg/mL polysorbate 80.
M491. 15-25 mg/mL EGFR/LGR5 BsAb; 3-12 mM histidine buffer system; a pH of 6.9-7.1; 100-320 mM sugar component; and 0.1-0.3 mg/mL polysorbate 80.
M492. 20 mg/mL EGFR/LGR5 BsAb; 3-12 mM histidine buffer system; a pH of 6.9-7.1; 100-320 mM sugar component; and 0.1-0.3 mg/mL polysorbate 80.
M493. 1-150 mg/mL EGFR/LGR5 BsAb; 5 mM histidine buffer system; a pH of 6.9-7.1; 100-320 mM sugar component; and 0.1-0.3 mg/mL polysorbate 80.
M494. 15-25 mg/mL EGFR/LGR5 BsAb; 5 mM histidine buffer system; a pH of 6.9-7.1; 100-320 mM sugar component; and 0.1-0.3 mg/mL polysorbate 80.
M495. 20 mg/mL EGFR/LGR5 BsAb; 5 mM histidine buffer system; a pH of 6.9-7.1; 100-320 mM sugar component; and 0.1-0.3 mg/mL polysorbate 80.
M496. 1-150 mg/mL EGFR/LGR5 BsAb; 10 mM histidine buffer system; a pH of 6.9-7.1; 100-320 mM sugar component; and 0.1-0.3 mg/mL polysorbate 80.
M497. 15-25 mg/mL EGFR/LGR5 BsAb; 10 mM histidine buffer system; a pH of 6.9-7.1; 100-320 mM sugar component; and 0.1-0.3 mg/mL polysorbate 80.
M498. 20 mg/mL EGFR/LGR5 BsAb; 10 mM histidine buffer system; a pH of 6.9-7.1; 100-320 mM sugar component; and 0.1-0.3 mg/mL polysorbate 80.
M499. 1-150 mg/mL EGFR/LGR5 BsAb; 3-12 mM citrate buffer system; a pH of 6.9-7.1; 100-320 mM sugar component; and 0.1-0.3 mg/mL polysorbate 80.
M500. 15-25 mg/mL EGFR/LGR5 BsAb; 3-12 mM citrate buffer system; a pH of 6.9-7.1; 100-320 mM sugar component; and 0.1-0.3 mg/mL polysorbate 80.
M501. 20 mg/mL EGFR/LGR5 BsAb; 3-12 mM citrate buffer system; a pH of 6.9-7.1; 100-320 mM sugar component; and 0.1-0.3 mg/mL polysorbate 80.
M502. 1-150 mg/mL EGFR/LGR5 BsAb; 10 mM citrate buffer system; a pH of 6.9-7.1; 100-320 mM sugar component; and 0.1-0.3 mg/mL polysorbate 80.
M503. 15-25 mg/mL EGFR/LGR5 BsAb; 10 mM citrate buffer system; a pH of 6.9-7.1; 100-320 mM sugar component; and 0.1-0.3 mg/mL polysorbate 80.
M504. 20 mg/mL EGFR/LGR5 BsAb; 10 mM citrate buffer system; a pH of 6.9-7.1; 100-320 mM sugar component; and 0.1-0.3 mg/mL polysorbate 80.
M505. 1-150 mg/mL EGFR/LGR5 BsAb; 2-70 mM buffer system; a pH of 5.4-7.2; 150-350 mM sucrose; and 0.1-0.3 mg/mL polysorbate 80.
M506. 15-25 mg/mL EGFR/LGR5 BsAb; 2-70 mM buffer system; a pH of 5.4-7.2; 150-350 mM sucrose; and 0.1-0.3 mg/mL polysorbate 80.
M507. 20 mg/mL EGFR/LGR5 BsAb; 2-70 mM buffer system; a pH of 5.4-7.2; 150-350 mM sucrose; and 0.1-0.3 mg/mL polysorbate 80.
M508. 1-150 mg/mL EGFR/LGR5 BsAb; 3-12 mM histidine buffer system; a pH of 5.4-7.2; 150-350 mM sucrose; and 0.1-0.3 mg/mL polysorbate 80.
M509. 15-25 mg/mL EGFR/LGR5 BsAb; 3-12 mM histidine buffer system; a pH of 5.4-7.2; 150-350 mM sucrose; and 0.1-0.3 mg/mL polysorbate 80.
M510. 20 mg/mL EGFR/LGR5 BsAb; 3-12 mM histidine buffer system; a pH of 5.4-7.2; 150-350 mM sucrose; and 0.1-0.3 mg/mL polysorbate 80.
M511. 1-150 mg/mL EGFR/LGR5 BsAb; 5 mM histidine buffer system; a pH of 5.4-7.2; 150-350 mM sucrose; and 0.1-0.3 mg/mL polysorbate 80.
M512. 15-25 mg/mL EGFR/LGR5 BsAb; 5 mM histidine buffer system; a pH of 5.4-7.2; 150-350 mM sucrose; and 0.1-0.3 mg/mL polysorbate 80.
M513. 20 mg/mL EGFR/LGR5 BsAb; 5 mM histidine buffer system; a pH of 5.4-7.2; 150-350 mM sucrose; and 0.1-0.3 mg/mL polysorbate 80.
M514. 1-150 mg/mL EGFR/LGR5 BsAb; 10 mM histidine buffer system; a pH of 5.4-7.2; 150-350 mM sucrose; and 0.1-0.3 mg/mL polysorbate 80.
M515. 15-25 mg/mL EGFR/LGR5 BsAb; 10 mM histidine buffer system; a pH of 5.4-7.2; 150-350 mM sucrose; and 0.1-0.3 mg/mL polysorbate 80.
M516. 20 mg/mL EGFR/LGR5 BsAb; 10 mM histidine buffer system; a pH of 5.4-7.2; 150-350 mM sucrose; and 0.1-0.3 mg/mL polysorbate 80.
M517. 1-150 mg/mL EGFR/LGR5 BsAb; 3-12 mM citrate buffer system; a pH of 5.8-7.8; 150-350 mM sucrose; and 0.1-0.3 mg/mL polysorbate 80.
M518. 15-25 mg/mL EGFR/LGR5 BsAb; 3-12 mM citrate buffer system; a pH of 5.8-7.8; 150-350 mM sucrose; and 0.1-0.3 mg/mL polysorbate 80.
M519. 20 mg/mL EGFR/LGR5 BsAb; 3-12 mM citrate buffer system; a pH of 5.8-7.8; 150-350 mM sucrose; and 0.1-0.3 mg/mL polysorbate 80.
M520. 1-150 mg/mL EGFR/LGR5 BsAb; 10 mM citrate buffer system; a pH of 5.8-7.8; 150-350 mM sucrose; and 0.1-0.3 mg/mL polysorbate 80.
M521. 15-25 mg/mL EGFR/LGR5 BsAb; 10 mM citrate buffer system; a pH of 5.8-7.8; 150-350 mM sucrose; and 0.1-0.3 mg/mL polysorbate 80.
M522. 20 mg/mL EGFR/LGR5 BsAb; 10 mM citrate buffer system; a pH of 5.8-7.8; 150-350 mM sucrose; and 0.1-0.3 mg/mL polysorbate 80.
M523. 1-150 mg/mL EGFR/LGR5 BsAb; 2-70 mM buffer system; a pH of 5.8-6.0; 150-350 mM sucrose; and 0.1-0.3 mg/mL polysorbate 80.
M524. 15-25 mg/mL EGFR/LGR5 BsAb; 2-70 mM buffer system; a pH of 5.8-6.0; 150-350 mM sucrose; and 0.1-0.3 mg/mL polysorbate 80.
M525. 20 mg/mL EGFR/LGR5 BsAb; 2-70 mM buffer system; a pH of 5.8-6.0; 150-350 mM sucrose; and 0.1-0.3 mg/mL polysorbate 80.
M526. 1-150 mg/mL EGFR/LGR5 BsAb; 3-12 mM histidine buffer system; a pH of 5.8-6.0; 150-350 mM sucrose; and 0.1-0.3 mg/mL polysorbate 80.
M527. 15-25 mg/mL EGFR/LGR5 BsAb; 3-12 mM histidine buffer system; a pH of 5.8-6.0; 150-350 mM sucrose; and 0.1-0.3 mg/mL polysorbate 80.
M528. 20 mg/mL EGFR/LGR5 BsAb; 3-12 mM histidine buffer system; a pH of 5.8-6.0; 150-350 mM sucrose; and 0.1-0.3 mg/mL polysorbate 80.
M529. 1-150 mg/mL EGFR/LGR5 BsAb; 5 mM histidine buffer system; a pH of 5.8-6.0; 150-350 mM sucrose; and 0.1-0.3 mg/mL polysorbate 80.
M530. 15-25 mg/mL EGFR/LGR5 BsAb; 5 mM histidine buffer system; a pH of 5.8-6.0; 150-350 mM sucrose; and 0.1-0.3 mg/mL polysorbate 80.
M531. 20 mg/mL EGFR/LGR5 BsAb; 5 mM histidine buffer system; a pH of 5.8-6.0; 150-350 mM sucrose; and 0.1-0.3 mg/mL polysorbate 80.
M532. 1-150 mg/mL EGFR/LGR5 BsAb; 10 mM histidine buffer system; a pH of 5.8-6.0; 150-350 mM sucrose; and 0.1-0.3 mg/mL polysorbate 80.
M533. 15-25 mg/mL EGFR/LGR5 BsAb; 10 mM histidine buffer system; a pH of 5.8-6.0; 150-350 mM sucrose; and 0.1-0.3 mg/mL polysorbate 80.
M534. 20 mg/mL EGFR/LGR5 BsAb; 10 mM histidine buffer system; a pH of 5.8-6.0; 150-350 mM sucrose; and 0.1-0.3 mg/mL polysorbate 80.
M535. 1-150 mg/mL EGFR/LGR5 BsAb; 3-12 mM citrate buffer system; a pH of 5.8-7; 150-350 mM sucrose; and 0.1-0.3 mg/mL polysorbate 80.
M536. 15-25 mg/mL EGFR/LGR5 BsAb; 3-12 mM citrate buffer system; a pH of 5.8-7; 150-350 mM sucrose; and 0.1-0.3 mg/mL polysorbate 80.
M537. 20 mg/mL EGFR/LGR5 BsAb; 3-12 mM citrate buffer system; a pH of 5.8-7; 150-350 mM sucrose; and 0.1-0.3 mg/mL polysorbate 80.
M538. 1-150 mg/mL EGFR/LGR5 BsAb; 10 mM citrate buffer system; a pH of 5.8-7; 150-350 mM sucrose; and 0.1-0.3 mg/mL polysorbate 80.
M539. 15-25 mg/mL EGFR/LGR5 BsAb; 10 mM citrate buffer system; a pH of 5.8-7; 150-350 mM sucrose; and 0.1-0.3 mg/mL polysorbate 80.
M540. 20 mg/mL EGFR/LGR5 BsAb; 10 mM citrate buffer system; a pH of 5.8-7; 150-350 mM sucrose; and 0.1-0.3 mg/mL polysorbate 80.
M541. 1-150 mg/mL EGFR/LGR5 BsAb; 2-70 mM buffer system; a pH of 6.2-6.4; 150-350 mM sucrose; and 0.1-0.3 mg/mL polysorbate 80.
M542. 15-25 mg/mL EGFR/LGR5 BsAb; 2-70 mM buffer system; a pH of 6.2-6.4; 150-350 mM sucrose; and 0.1-0.3 mg/mL polysorbate 80.
M543. 20 mg/mL EGFR/LGR5 BsAb; 2-70 mM buffer system; a pH of 6.2-6.4; 150-350 mM sucrose; and 0.1-0.3 mg/mL polysorbate 80.
M544. 1-150 mg/mL EGFR/LGR5 BsAb; 3-12 mM histidine buffer system; a pH of 6.2-6.4; 150-350 mM sucrose; and 0.1-0.3 mg/mL polysorbate 80.
M545. 15-25 mg/mL EGFR/LGR5 BsAb; 3-12 mM histidine buffer system; a pH of 6.2-6.4; 150-350 mM sucrose; and 0.1-0.3 mg/mL polysorbate 80.
M546. 20 mg/mL EGFR/LGR5 BsAb; 3-12 mM histidine buffer system; a pH of 6.2-6.4; 150-350 mM sucrose; and 0.1-0.3 mg/mL polysorbate 80.
M547. 1-150 mg/mL EGFR/LGR5 BsAb; 5 mM histidine buffer system; a pH of 6.2-6.4; 150-350 mM sucrose; and 0.1-0.3 mg/mL polysorbate 80.
M548. 15-25 mg/mL EGFR/LGR5 BsAb; 5 mM histidine buffer system; a pH of 6.2-6.4; 150-350 mM sucrose; and 0.1-0.3 mg/mL polysorbate 80.
M549. 20 mg/mL EGFR/LGR5 BsAb; 5 mM histidine buffer system; a pH of 6.2-6.4; 150-350 mM sucrose; and 0.1-0.3 mg/mL polysorbate 80.
M550. 1-150 mg/mL EGFR/LGR5 BsAb; 10 mM histidine buffer system; a pH of 6.2-6.4; 150-350 mM sucrose; and 0.1-0.3 mg/mL polysorbate 80.
M551. 15-25 mg/mL EGFR/LGR5 BsAb; 10 mM histidine buffer system; a pH of 6.2-6.4; 150-350 mM sucrose; and 0.1-0.3 mg/mL polysorbate 80.
M552. 20 mg/mL EGFR/LGR5 BsAb; 10 mM histidine buffer system; a pH of 6.2-6.4; 150-350 mM sucrose; and 0.1-0.3 mg/mL polysorbate 80.
M553. 1-150 mg/mL EGFR/LGR5 BsAb; 3-12 mM citrate buffer system; a pH of 6.2-7.4; 150-350 mM sucrose; and 0.1-0.3 mg/mL polysorbate 80.
M554. 15-25 mg/mL EGFR/LGR5 BsAb; 3-12 mM citrate buffer system; a pH of 6.2-7.4; 150-350 mM sucrose; and 0.1-0.3 mg/mL polysorbate 80.
M555. 20 mg/mL EGFR/LGR5 BsAb; 3-12 mM citrate buffer system; a pH of 6.2-7.4; 150-350 mM sucrose; and 0.1-0.3 mg/mL polysorbate 80.
M556. 1-150 mg/mL EGFR/LGR5 BsAb; 10 mM citrate buffer system; a pH of 6.2-7.4; 150-350 mM sucrose; and 0.1-0.3 mg/mL polysorbate 80.
M557. 15-25 mg/mL EGFR/LGR5 BsAb; 10 mM citrate buffer system; a pH of 6.2-7.4; 150-350 mM sucrose; and 0.1-0.3 mg/mL polysorbate 80.
M558. 20 mg/mL EGFR/LGR5 BsAb; 10 mM citrate buffer system; a pH of 6.2-7.4; 150-350 mM sucrose; and 0.1-0.3 mg/mL polysorbate 80.
M559. 1-150 mg/mL EGFR/LGR5 BsAb; 2-70 mM buffer system; a pH of 6.9-7.1; 150-350 mM sucrose; and 0.1-0.3 mg/mL polysorbate 80.
M560. 15-25 mg/mL EGFR/LGR5 BsAb; 2-70 mM buffer system; a pH of 6.9-7.1; 150-350 mM sucrose; and 0.1-0.3 mg/mL polysorbate 80.
M561. 20 mg/mL EGFR/LGR5 BsAb; 2-70 mM buffer system; a pH of 6.9-7.1; 150-350 mM sucrose; and 0.1-0.3 mg/mL polysorbate 80.
M562. 1-150 mg/mL EGFR/LGR5 BsAb; 3-12 mM histidine buffer system; a pH of 6.9-7.1; 150-350 mM sucrose; and 0.1-0.3 mg/mL polysorbate 80.
M563. 15-25 mg/mL EGFR/LGR5 BsAb; 3-12 mM histidine buffer system; a pH of 6.9-7.1; 150-350 mM sucrose; and 0.1-0.3 mg/mL polysorbate 80.
M564. 20 mg/mL EGFR/LGR5 BsAb; 3-12 mM histidine buffer system; a pH of 6.9-7.1; 150-350 mM sucrose; and 0.1-0.3 mg/mL polysorbate 80.
M565. 1-150 mg/mL EGFR/LGR5 BsAb; 5 mM histidine buffer system; a pH of 6.9-7.1; 150-350 mM sucrose; and 0.1-0.3 mg/mL polysorbate 80.
M566. 15-25 mg/mL EGFR/LGR5 BsAb; 5 mM histidine buffer system; a pH of 6.9-7.1; 150-350 mM sucrose; and 0.1-0.3 mg/mL polysorbate 80.
M567. 20 mg/mL EGFR/LGR5 BsAb; 5 mM histidine buffer system; a pH of 6.9-7.1; 150-350 mM sucrose; and 0.1-0.3 mg/mL polysorbate 80.
M568. 1-150 mg/mL EGFR/LGR5 BsAb; 10 mM histidine buffer system; a pH of 6.9-7.1; 150-350 mM sucrose; and 0.1-0.3 mg/mL polysorbate 80.
M569. 15-25 mg/mL EGFR/LGR5 BsAb; 10 mM histidine buffer system; a pH of 6.9-7.1; 150-350 mM sucrose; and 0.1-0.3 mg/mL polysorbate 80.
M570. 20 mg/mL EGFR/LGR5 BsAb; 10 mM histidine buffer system; a pH of 6.9-7.1; 150-350 mM sucrose; and 0.1-0.3 mg/mL polysorbate 80.
M571. 1-150 mg/mL EGFR/LGR5 BsAb; 3-12 mM citrate buffer system; a pH of 6.9-7.1; 150-350 mM sucrose; and 0.1-0.3 mg/mL polysorbate 80.
M572. 15-25 mg/mL EGFR/LGR5 BsAb; 3-12 mM citrate buffer system; a pH of 6.9-7.1; 150-350 mM sucrose; and 0.1-0.3 mg/mL polysorbate 80.
M573. 20 mg/mL EGFR/LGR5 BsAb; 3-12 mM citrate buffer system; a pH of 6.9-7.1; 150-350 mM sucrose; and 0.1-0.3 mg/mL polysorbate 80.
M574. 1-150 mg/mL EGFR/LGR5 BsAb; 10 mM citrate buffer system; a pH of 6.9-7.1; 150-350 mM sucrose; and 0.1-0.3 mg/mL polysorbate 80.
M575. 15-25 mg/mL EGFR/LGR5 BsAb; 10 mM citrate buffer system; a pH of 6.9-7.1; 150-350 mM sucrose; and 0.1-0.3 mg/mL polysorbate 80.
M576. 20 mg/mL EGFR/LGR5 BsAb; 10 mM citrate buffer system; a pH of 6.9-7.1; 150-350 mM sucrose; and 0.1-0.3 mg/mL polysorbate 80.
M577. 1-150 mg/mL EGFR/LGR5 BsAb; 2-70 mM buffer system; a pH of 5.4-7.2; 275-295 mM sucrose; and 0.1-0.3 mg/mL polysorbate 80.
M578. 15-25 mg/mL EGFR/LGR5 BsAb; 2-70 mM buffer system; a pH of 5.4-7.2; 275-295 mM sucrose; and 0.1-0.3 mg/mL polysorbate 80.
M579. 20 mg/mL EGFR/LGR5 BsAb; 2-70 mM buffer system; a pH of 5.4-7.2; 275-295 mM sucrose; and 0.1-0.3 mg/mL polysorbate 80.
M580. 1-150 mg/mL EGFR/LGR5 BsAb; 3-12 mM histidine buffer system; a pH of 5.4-7.2; 275-295 mM sucrose; and 0.1-0.3 mg/mL polysorbate 80.
M581. 15-25 mg/mL EGFR/LGR5 BsAb; 3-12 mM histidine buffer system; a pH of 5.4-7.2; 275-295 mM sucrose; and 0.1-0.3 mg/mL polysorbate 80.
M582. 20 mg/mL EGFR/LGR5 BsAb; 3-12 mM histidine buffer system; a pH of 5.4-7.2; 275-295 mM sucrose; and 0.1-0.3 mg/mL polysorbate 80.
M583. 1-150 mg/mL EGFR/LGR5 BsAb; 5 mM histidine buffer system; a pH of 5.4-7.2; 275-295 mM sucrose; and 0.1-0.3 mg/mL polysorbate 80.
M584. 15-25 mg/mL EGFR/LGR5 BsAb; 5 mM histidine buffer system; a pH of 5.4-7.2; 275-295 mM sucrose; and 0.1-0.3 mg/mL polysorbate 80.
M585. 20 mg/mL EGFR/LGR5 BsAb; 5 mM histidine buffer system; a pH of 5.4-7.2; 275-295 mM sucrose; and 0.1-0.3 mg/mL polysorbate 80.
M586. 1-150 mg/mL EGFR/LGR5 BsAb; 10 mM histidine buffer system; a pH of 5.4-7.2; 275-295 mM sucrose; and 0.1-0.3 mg/mL polysorbate 80.
M587. 15-25 mg/mL EGFR/LGR5 BsAb; 10 mM histidine buffer system; a pH of 5.4-7.2; 275-295 mM sucrose; and 0.1-0.3 mg/mL polysorbate 80.
M588. 20 mg/mL EGFR/LGR5 BsAb; 10 mM histidine buffer system; a pH of 5.4-7.2; 275-295 mM sucrose; and 0.1-0.3 mg/mL polysorbate 80.
M589. 1-150 mg/mL EGFR/LGR5 BsAb; 3-12 mM citrate buffer system; a pH of 5.8-7.8; 275-295 mM sucrose; and 0.1-0.3 mg/mL polysorbate 80.
M590. 15-25 mg/mL EGFR/LGR5 BsAb; 3-12 mM citrate buffer system; a pH of 5.8-7.8; 275-295 mM sucrose; and 0.1-0.3 mg/mL polysorbate 80.
M591. 20 mg/mL EGFR/LGR5 BsAb; 3-12 mM citrate buffer system; a pH of 5.8-7.8; 275-295 mM sucrose; and 0.1-0.3 mg/mL polysorbate 80.
M592. 1-150 mg/mL EGFR/LGR5 BsAb; 10 mM citrate buffer system; a pH of 5.8-7.8; 275-295 mM sucrose; and 0.1-0.3 mg/mL polysorbate 80.
M593. 15-25 mg/mL EGFR/LGR5 BsAb; 10 mM citrate buffer system; a pH of 5.8-7.8; 275-295 mM sucrose; and 0.1-0.3 mg/mL polysorbate 80.
M594. 20 mg/mL EGFR/LGR5 BsAb; 10 mM citrate buffer system; a pH of 5.8-7.8; 275-295 mM sucrose; and 0.1-0.3 mg/mL polysorbate 80.
M595. 1-150 mg/mL EGFR/LGR5 BsAb; 2-70 mM buffer system; a pH of 5.8-6.0; 275-295 mM sucrose; and 0.1-0.3 mg/mL polysorbate 80.
M596. 15-25 mg/mL EGFR/LGR5 BsAb; 2-70 mM buffer system; a pH of 5.8-6.0; 275-295 mM sucrose; and 0.1-0.3 mg/mL polysorbate 80.
M597. 20 mg/mL EGFR/LGR5 BsAb; 2-70 mM buffer system; a pH of 5.8-6.0; 275-295 mM sucrose; and 0.1-0.3 mg/mL polysorbate 80.
M598. 1-150 mg/mL EGFR/LGR5 BsAb; 3-12 mM histidine buffer system; a pH of 5.8-6.0; 275-295 mM sucrose; and 0.1-0.3 mg/mL polysorbate 80.
M599. 15-25 mg/mL EGFR/LGR5 BsAb; 3-12 mM histidine buffer system; a pH of 5.8-6.0; 275-295 mM sucrose; and 0.1-0.3 mg/mL polysorbate 80.
M600. 20 mg/mL EGFR/LGR5 BsAb; 3-12 mM histidine buffer system; a pH of 5.8-6.0; 275-295 mM sucrose; and 0.1-0.3 mg/mL polysorbate 80.
M601. 1-150 mg/mL EGFR/LGR5 BsAb; 5 mM histidine buffer system; a pH of 5.8-6.0; 275-295 mM sucrose; and 0.1-0.3 mg/mL polysorbate 80.
M602. 15-25 mg/mL EGFR/LGR5 BsAb; 5 mM histidine buffer system; a pH of 5.8-6.0; 275-295 mM sucrose; and 0.1-0.3 mg/mL polysorbate 80.
M603. 20 mg/mL EGFR/LGR5 BsAb; 5 mM histidine buffer system; a pH of 5.8-6.0; 275-295 mM sucrose; and 0.1-0.3 mg/mL polysorbate 80.
M604. 1-150 mg/mL EGFR/LGR5 BsAb; 10 mM histidine buffer system; a pH of 5.8-6.0; 275-295 mM sucrose; and 0.1-0.3 mg/mL polysorbate 80.
M605. 15-25 mg/mL EGFR/LGR5 BsAb; 10 mM histidine buffer system; a pH of 5.8-6.0; 275-295 mM sucrose; and 0.1-0.3 mg/mL polysorbate 80.
M606. 20 mg/mL EGFR/LGR5 BsAb; 10 mM histidine buffer system; a pH of 5.8-6.0; 275-295 mM sucrose; and 0.1-0.3 mg/mL polysorbate 80.
M607. 1-150 mg/mL EGFR/LGR5 BsAb; 3-12 mM citrate buffer system; a pH of 5.8-7; 275-295 mM sucrose; and 0.1-0.3 mg/mL polysorbate 80.
M608. 15-25 mg/mL EGFR/LGR5 BsAb; 3-12 mM citrate buffer system; a pH of 5.8-7; 275-295 mM sucrose; and 0.1-0.3 mg/mL polysorbate 80.
M609. 20 mg/mL EGFR/LGR5 BsAb; 3-12 mM citrate buffer system; a pH of 5.8-7; 275-295 mM sucrose; and 0.1-0.3 mg/mL polysorbate 80.
M610. 1-150 mg/mL EGFR/LGR5 BsAb; 10 mM citrate buffer system; a pH of 5.8-7; 275-295 mM sucrose; and 0.1-0.3 mg/mL polysorbate 80.
M611. 15-25 mg/mL EGFR/LGR5 BsAb; 10 mM citrate buffer system; a pH of 5.8-7; 275-295 mM sucrose; and 0.1-0.3 mg/mL polysorbate 80.
M612. 20 mg/mL EGFR/LGR5 BsAb; 10 mM citrate buffer system; a pH of 5.8-7; 275-295 mM sucrose; and 0.1-0.3 mg/mL polysorbate 80.
M613. 1-150 mg/mL EGFR/LGR5 BsAb; 2-70 mM buffer system; a pH of 6.2-6.4; 275-295 mM sucrose; and 0.1-0.3 mg/mL polysorbate 80.
M614. 15-25 mg/mL EGFR/LGR5 BsAb; 2-70 mM buffer system; a pH of 6.2-6.4; 275-295 mM sucrose; and 0.1-0.3 mg/mL polysorbate 80.
M615. 20 mg/mL EGFR/LGR5 BsAb; 2-70 mM buffer system; a pH of 6.2-6.4; 275-295 mM sucrose; and 0.1-0.3 mg/mL polysorbate 80.
M616. 1-150 mg/mL EGFR/LGR5 BsAb; 3-12 mM histidine buffer system; a pH of 6.2-6.4; 275-295 mM sucrose; and 0.1-0.3 mg/mL polysorbate 80.
M617. 15-25 mg/mL EGFR/LGR5 BsAb; 3-12 mM histidine buffer system; a pH of 6.2-6.4; 275-295 mM sucrose; and 0.1-0.3 mg/mL polysorbate 80.
M618. 20 mg/mL EGFR/LGR5 BsAb; 3-12 mM histidine buffer system; a pH of 6.2-6.4; 275-295 mM sucrose; and 0.1-0.3 mg/mL polysorbate 80.
M619. 1-150 mg/mL EGFR/LGR5 BsAb; 5 mM histidine buffer system; a pH of 6.2-6.4; 275-295 mM sucrose; and 0.1-0.3 mg/mL polysorbate 80.
M620. 15-25 mg/mL EGFR/LGR5 BsAb; 5 mM histidine buffer system; a pH of 6.2-6.4; 275-295 mM sucrose; and 0.1-0.3 mg/mL polysorbate 80.
M621. 20 mg/mL EGFR/LGR5 BsAb; 5 mM histidine buffer system; a pH of 6.2-6.4; 275-295 mM sucrose; and 0.1-0.3 mg/mL polysorbate 80.
M622. 1-150 mg/mL EGFR/LGR5 BsAb; 10 mM histidine buffer system; a pH of 6.2-6.4; 275-295 mM sucrose; and 0.1-0.3 mg/mL polysorbate 80.
M623. 15-25 mg/mL EGFR/LGR5 BsAb; 10 mM histidine buffer system; a pH of 6.2-6.4; 275-295 mM sucrose; and 0.1-0.3 mg/mL polysorbate 80.
M624. 20 mg/mL EGFR/LGR5 BsAb; 10 mM histidine buffer system; a pH of 6.2-6.4; 275-295 mM sucrose; and 0.1-0.3 mg/mL polysorbate 80.
M625. 1-150 mg/mL EGFR/LGR5 BsAb; 3-12 mM citrate buffer system; a pH of 6.2-7.4; 275-295 mM sucrose; and 0.1-0.3 mg/mL polysorbate 80.
M626. 15-25 mg/mL EGFR/LGR5 BsAb; 3-12 mM citrate buffer system; a pH of 6.2-7.4; 275-295 mM sucrose; and 0.1-0.3 mg/mL polysorbate 80.
M627. 20 mg/mL EGFR/LGR5 BsAb; 3-12 mM citrate buffer system; a pH of 6.2-7.4; 275-295 mM sucrose; and 0.1-0.3 mg/mL polysorbate 80.
M628. 1-150 mg/mL EGFR/LGR5 BsAb; 10 mM citrate buffer system; a pH of 6.2-7.4; 275-295 mM sucrose; and 0.1-0.3 mg/mL polysorbate 80.
M629. 15-25 mg/mL EGFR/LGR5 BsAb; 10 mM citrate buffer system; a pH of 6.2-7.4; 275-295 mM sucrose; and 0.1-0.3 mg/mL polysorbate 80.
M630. 20 mg/mL EGFR/LGR5 BsAb; 10 mM citrate buffer system; a pH of 6.2-7.4; 275-295 mM sucrose; and 0.1-0.3 mg/mL polysorbate 80.
M631. 1-150 mg/mL EGFR/LGR5 BsAb; 2-70 mM buffer system; a pH of 6.9-7.1; 275-295 mM sucrose; and 0.1-0.3 mg/mL polysorbate 80.
M632. 15-25 mg/mL EGFR/LGR5 BsAb; 2-70 mM buffer system; a pH of 6.9-7.1; 275-295 mM sucrose; and 0.1-0.3 mg/mL polysorbate 80.
M633. 20 mg/mL EGFR/LGR5 BsAb; 2-70 mM buffer system; a pH of 6.9-7.1; 275-295 mM sucrose; and 0.1-0.3 mg/mL polysorbate 80.
M634. 1-150 mg/mL EGFR/LGR5 BsAb; 3-12 mM histidine buffer system; a pH of 6.9-7.1; 275-295 mM sucrose; and 0.1-0.3 mg/mL polysorbate 80.
M635. 15-25 mg/mL EGFR/LGR5 BsAb; 3-12 mM histidine buffer system; a pH of 6.9-7.1; 275-295 mM sucrose; and 0.1-0.3 mg/mL polysorbate 80.
M636. 20 mg/mL EGFR/LGR5 BsAb; 3-12 mM histidine buffer system; a pH of 6.9-7.1; 275-295 mM sucrose; and 0.1-0.3 mg/mL polysorbate 80.
M637. 1-150 mg/mL EGFR/LGR5 BsAb; 5 mM histidine buffer system; a pH of 6.9-7.1; 275-295 mM sucrose; and 0.1-0.3 mg/mL polysorbate 80.
M638. 15-25 mg/mL EGFR/LGR5 BsAb; 5 mM histidine buffer system; a pH of 6.9-7.1; 275-295 mM sucrose; and 0.1-0.3 mg/mL polysorbate 80.
M639. 20 mg/mL EGFR/LGR5 BsAb; 5 mM histidine buffer system; a pH of 6.9-7.1; 275-295 mM sucrose; and 0.1-0.3 mg/mL polysorbate 80.
M640. 1-150 mg/mL EGFR/LGR5 BsAb; 10 mM histidine buffer system; a pH of 6.9-7.1; 275-295 mM sucrose; and 0.1-0.3 mg/mL polysorbate 80.
M641. 15-25 mg/mL EGFR/LGR5 BsAb; 10 mM histidine buffer system; a pH of 6.9-7.1; 275-295 mM sucrose; and 0.1-0.3 mg/mL polysorbate 80.
M642. 20 mg/mL EGFR/LGR5 BsAb; 10 mM histidine buffer system; a pH of 6.9-7.1; 275-295 mM sucrose; and 0.1-0.3 mg/mL polysorbate 80.
M643. 1-150 mg/mL EGFR/LGR5 BsAb; 3-12 mM citrate buffer system; a pH of 6.9-7.1; 275-295 mM sucrose; and 0.1-0.3 mg/mL polysorbate 80.
M644. 15-25 mg/mL EGFR/LGR5 BsAb; 3-12 mM citrate buffer system; a pH of 6.9-7.1; 275-295 mM sucrose; and 0.1-0.3 mg/mL polysorbate 80.
M645. 20 mg/mL EGFR/LGR5 BsAb; 3-12 mM citrate buffer system; a pH of 6.9-7.1; 275-295 mM sucrose; and 0.1-0.3 mg/mL polysorbate 80.
M646. 1-150 mg/mL EGFR/LGR5 BsAb; 10 mM citrate buffer system; a pH of 6.9-7.1; 275-295 mM sucrose; and 0.1-0.3 mg/mL polysorbate 80.
M647. 15-25 mg/mL EGFR/LGR5 BsAb; 10 mM citrate buffer system; a pH of 6.9-7.1; 275-295 mM sucrose; and 0.1-0.3 mg/mL polysorbate 80.
M648. 20 mg/mL EGFR/LGR5 BsAb; 10 mM citrate buffer system; a pH of 6.9-7.1; 275-295 mM sucrose; and 0.1-0.3 mg/mL polysorbate 80.
M649. 1-150 mg/mL EGFR/LGR5 BsAb; 2-70 mM buffer system; a pH of 5.4-7.2; 100-320 mM sugar component; and 0.4-0.6 mg/mL polysorbate 80.
M650. 15-25 mg/mL EGFR/LGR5 BsAb; 2-70 mM buffer system; a pH of 5.4-7.2; 100-320 mM sugar component; and 0.4-0.6 mg/mL polysorbate 80.
M651. 20 mg/mL EGFR/LGR5 BsAb; 2-70 mM buffer system; a pH of 5.4-7.2; 100-320 mM sugar component; and 0.4-0.6 mg/mL polysorbate 80.
M652. 1-150 mg/mL EGFR/LGR5 BsAb; 3-12 mM histidine buffer system; a pH of 5.4-7.2; 100-320 mM sugar component; and 0.4-0.6 mg/mL polysorbate 80.
M653. 15-25 mg/mL EGFR/LGR5 BsAb; 3-12 mM histidine buffer system; a pH of 5.4-7.2; 100-320 mM sugar component; and 0.4-0.6 mg/mL polysorbate 80.
M654. 20 mg/mL EGFR/LGR5 BsAb; 3-12 mM histidine buffer system; a pH of 5.4-7.2; 100-320 mM sugar component; and 0.4-0.6 mg/mL polysorbate 80.
M655. 1-150 mg/mL EGFR/LGR5 BsAb; 5 mM histidine buffer system; a pH of 5.4-7.2; 100-320 mM sugar component; and 0.4-0.6 mg/mL polysorbate 80.
M656. 15-25 mg/mL EGFR/LGR5 BsAb; 5 mM histidine buffer system; a pH of 5.4-7.2; 100-320 mM sugar component; and 0.4-0.6 mg/mL polysorbate 80.
M657. 20 mg/mL EGFR/LGR5 BsAb; 5 mM histidine buffer system; a pH of 5.4-7.2; 100-320 mM sugar component; and 0.4-0.6 mg/mL polysorbate 80.
M658. 1-150 mg/mL EGFR/LGR5 BsAb; 10 mM histidine buffer system; a pH of 5.4-7.2; 100-320 mM sugar component; and 0.4-0.6 mg/mL polysorbate 80.
M659. 15-25 mg/mL EGFR/LGR5 BsAb; 10 mM histidine buffer system; a pH of 5.4-7.2; 100-320 mM sugar component; and 0.4-0.6 mg/mL polysorbate 80.
M660. 20 mg/mL EGFR/LGR5 BsAb; 10 mM histidine buffer system; a pH of 5.4-7.2; 100-320 mM sugar component; and 0.4-0.6 mg/mL polysorbate 80.
M661. 1-150 mg/mL EGFR/LGR5 BsAb; 3-12 mM citrate buffer system; a pH of 5.8-7.8; 100-320 mM sugar component; and 0.4-0.6 mg/mL polysorbate 80.
M662. 15-25 mg/mL EGFR/LGR5 BsAb; 3-12 mM citrate buffer system; a pH of 5.8-7.8; 100-320 mM sugar component; and 0.4-0.6 mg/mL polysorbate 80.
M663. 20 mg/mL EGFR/LGR5 BsAb; 3-12 mM citrate buffer system; a pH of 5.8-7.8; 100-320 mM sugar component; and 0.4-0.6 mg/mL polysorbate 80.
M664. 1-150 mg/mL EGFR/LGR5 BsAb; 10 mM citrate buffer system; a pH of 5.8-7.8; 100-320 mM sugar component; and 0.4-0.6 mg/mL polysorbate 80.
M665. 15-25 mg/mL EGFR/LGR5 BsAb; 10 mM citrate buffer system; a pH of 5.8-7.8; 100-320 mM sugar component; and 0.4-0.6 mg/mL polysorbate 80.
M666. 20 mg/mL EGFR/LGR5 BsAb; 10 mM citrate buffer system; a pH of 5.8-7.8; 100-320 mM sugar component; and 0.4-0.6 mg/mL polysorbate 80.
M667. 1-150 mg/mL EGFR/LGR5 BsAb; 2-70 mM buffer system; a pH of 5.8-6.0; 100-320 mM sugar component; and 0.4-0.6 mg/mL polysorbate 80.
M668. 15-25 mg/mL EGFR/LGR5 BsAb; 2-70 mM buffer system; a pH of 5.8-6.0; 100-320 mM sugar component; and 0.4-0.6 mg/mL polysorbate 80.
M669. 20 mg/mL EGFR/LGR5 BsAb; 2-70 mM buffer system; a pH of 5.8-6.0; 100-320 mM sugar component; and 0.4-0.6 mg/mL polysorbate 80.
M670. 1-150 mg/mL EGFR/LGR5 BsAb; 3-12 mM histidine buffer system; a pH of 5.8-6.0; 100-320 mM sugar component; and 0.4-0.6 mg/mL polysorbate 80.
M671. 15-25 mg/mL EGFR/LGR5 BsAb; 3-12 mM histidine buffer system; a pH of 5.8-6.0; 100-320 mM sugar component; and 0.4-0.6 mg/mL polysorbate 80.
M672. 20 mg/mL EGFR/LGR5 BsAb; 3-12 mM histidine buffer system; a pH of 5.8-6.0; 100-320 mM sugar component; and 0.4-0.6 mg/mL polysorbate 80.
M673. 1-150 mg/mL EGFR/LGR5 BsAb; 5 mM histidine buffer system; a pH of 5.8-6.0; 100-320 mM sugar component; and 0.4-0.6 mg/mL polysorbate 80.
M674. 15-25 mg/mL EGFR/LGR5 BsAb; 5 mM histidine buffer system; a pH of 5.8-6.0; 100-320 mM sugar component; and 0.4-0.6 mg/mL polysorbate 80.
M675. 20 mg/mL EGFR/LGR5 BsAb; 5 mM histidine buffer system; a pH of 5.8-6.0; 100-320 mM sugar component; and 0.4-0.6 mg/mL polysorbate 80.
M676. 1-150 mg/mL EGFR/LGR5 BsAb; 10 mM histidine buffer system; a pH of 5.8-6.0; 100-320 mM sugar component; and 0.4-0.6 mg/mL polysorbate 80.
M677. 15-25 mg/mL EGFR/LGR5 BsAb; 10 mM histidine buffer system; a pH of 5.8-6.0; 100-320 mM sugar component; and 0.4-0.6 mg/mL polysorbate 80.
M678. 20 mg/mL EGFR/LGR5 BsAb; 10 mM histidine buffer system; a pH of 5.8-6.0; 100-320 mM sugar component; and 0.4-0.6 mg/mL polysorbate 80.
M679. 1-150 mg/mL EGFR/LGR5 BsAb; 3-12 mM citrate buffer system; a pH of 5.8-7; 100-320 mM sugar component; and 0.4-0.6 mg/mL polysorbate 80.
M680. 15-25 mg/mL EGFR/LGR5 BsAb; 3-12 mM citrate buffer system; a pH of 5.8-7; 100-320 mM sugar component; and 0.4-0.6 mg/mL polysorbate 80.
M681. 20 mg/mL EGFR/LGR5 BsAb; 3-12 mM citrate buffer system; a pH of 5.8-7; 100-320 mM sugar component; and 0.4-0.6 mg/mL polysorbate 80.
M682. 1-150 mg/mL EGFR/LGR5 BsAb; 10 mM citrate buffer system; a pH of 5.8-7; 100-320 mM sugar component; and 0.4-0.6 mg/mL polysorbate 80.
M683. 15-25 mg/mL EGFR/LGR5 BsAb; 10 mM citrate buffer system; a pH of 5.8-7; 100-320 mM sugar component; and 0.4-0.6 mg/mL polysorbate 80.
M684. 20 mg/mL EGFR/LGR5 BsAb; 10 mM citrate buffer system; a pH of 5.8-7; 100-320 mM sugar component; and 0.4-0.6 mg/mL polysorbate 80.
M685. 1-150 mg/mL EGFR/LGR5 BsAb; 2-70 mM buffer system; a pH of 6.2-6.4; 100-320 mM sugar component; and 0.4-0.6 mg/mL polysorbate 80.
M686. 15-25 mg/mL EGFR/LGR5 BsAb; 2-70 mM buffer system; a pH of 6.2-6.4; 100-320 mM sugar component; and 0.4-0.6 mg/mL polysorbate 80.
M687. 20 mg/mL EGFR/LGR5 BsAb; 2-70 mM buffer system; a pH of 6.2-6.4; 100-320 mM sugar component; and 0.4-0.6 mg/mL polysorbate 80.
M688. 1-150 mg/mL EGFR/LGR5 BsAb; 3-12 mM histidine buffer system; a pH of 6.2-6.4; 100-320 mM sugar component; and 0.4-0.6 mg/mL polysorbate 80.
M689. 15-25 mg/mL EGFR/LGR5 BsAb; 3-12 mM histidine buffer system; a pH of 6.2-6.4; 100-320 mM sugar component; and 0.4-0.6 mg/mL polysorbate 80.
M690. 20 mg/mL EGFR/LGR5 BsAb; 3-12 mM histidine buffer system; a pH of 6.2-6.4; 100-320 mM sugar component; and 0.4-0.6 mg/mL polysorbate 80.
M691. 1-150 mg/mL EGFR/LGR5 BsAb; 5 mM histidine buffer system; a pH of 6.2-6.4; 100-320 mM sugar component; and 0.4-0.6 mg/mL polysorbate 80.
M692. 15-25 mg/mL EGFR/LGR5 BsAb; 5 mM histidine buffer system; a pH of 6.2-6.4; 100-320 mM sugar component; and 0.4-0.6 mg/mL polysorbate 80.
M693. 20 mg/mL EGFR/LGR5 BsAb; 5 mM histidine buffer system; a pH of 6.2-6.4; 100-320 mM sugar component; and 0.4-0.6 mg/mL polysorbate 80.
M694. 1-150 mg/mL EGFR/LGR5 BsAb; 10 mM histidine buffer system; a pH of 6.2-6.4; 100-320 mM sugar component; and 0.4-0.6 mg/mL polysorbate 80.
M695. 15-25 mg/mL EGFR/LGR5 BsAb; 10 mM histidine buffer system; a pH of 6.2-6.4; 100-320 mM sugar component; and 0.4-0.6 mg/mL polysorbate 80.
M696. 20 mg/mL EGFR/LGR5 BsAb; 10 mM histidine buffer system; a pH of 6.2-6.4; 100-320 mM sugar component; and 0.4-0.6 mg/mL polysorbate 80.
M697. 1-150 mg/mL EGFR/LGR5 BsAb; 3-12 mM citrate buffer system; a pH of 6.2-7.4; 100-320 mM sugar component; and 0.4-0.6 mg/mL polysorbate 80.
M698. 15-25 mg/mL EGFR/LGR5 BsAb; 3-12 mM citrate buffer system; a pH of 6.2-7.4; 100-320 mM sugar component; and 0.4-0.6 mg/mL polysorbate 80.
M699. 20 mg/mL EGFR/LGR5 BsAb; 3-12 mM citrate buffer system; a pH of 6.2-7.4; 100-320 mM sugar component; and 0.4-0.6 mg/mL polysorbate 80.
M700. 1-150 mg/mL EGFR/LGR5 BsAb; 10 mM citrate buffer system; a pH of 6.2-7.4; 100-320 mM sugar component; and 0.4-0.6 mg/mL polysorbate 80.
M701. 15-25 mg/mL EGFR/LGR5 BsAb; 10 mM citrate buffer system; a pH of 6.2-7.4; 100-320 mM sugar component; and 0.4-0.6 mg/mL polysorbate 80.
M702. 20 mg/mL EGFR/LGR5 BsAb; 10 mM citrate buffer system; a pH of 6.2-7.4; 100-320 mM sugar component; and 0.4-0.6 mg/mL polysorbate 80.
M703. 1-150 mg/mL EGFR/LGR5 BsAb; 2-70 mM buffer system; a pH of 6.9-7.1; 100-320 mM sugar component; and 0.4-0.6 mg/mL polysorbate 80.
M704. 15-25 mg/mL EGFR/LGR5 BsAb; 2-70 mM buffer system; a pH of 6.9-7.1; 100-320 mM sugar component; and 0.4-0.6 mg/mL polysorbate 80.
M705. 20 mg/mL EGFR/LGR5 BsAb; 2-70 mM buffer system; a pH of 6.9-7.1; 100-320 mM sugar component; and 0.4-0.6 mg/mL polysorbate 80.
M706. 1-150 mg/mL EGFR/LGR5 BsAb; 3-12 mM histidine buffer system; a pH of 6.9-7.1; 100-320 mM sugar component; and 0.4-0.6 mg/mL polysorbate 80.
M707. 15-25 mg/mL EGFR/LGR5 BsAb; 3-12 mM histidine buffer system; a pH of 6.9-7.1; 100-320 mM sugar component; and 0.4-0.6 mg/mL polysorbate 80.
M708. 20 mg/mL EGFR/LGR5 BsAb; 3-12 mM histidine buffer system; a pH of 6.9-7.1; 100-320 mM sugar component; and 0.4-0.6 mg/mL polysorbate 80.
M709. 1-150 mg/mL EGFR/LGR5 BsAb; 5 mM histidine buffer system; a pH of 6.9-7.1; 100-320 mM sugar component; and 0.4-0.6 mg/mL polysorbate 80.
M710. 15-25 mg/mL EGFR/LGR5 BsAb; 5 mM histidine buffer system; a pH of 6.9-7.1; 100-320 mM sugar component; and 0.4-0.6 mg/mL polysorbate 80.
M711. 20 mg/mL EGFR/LGR5 BsAb; 5 mM histidine buffer system; a pH of 6.9-7.1; 100-320 mM sugar component; and 0.4-0.6 mg/mL polysorbate 80.
M712. 1-150 mg/mL EGFR/LGR5 BsAb; 10 mM histidine buffer system; a pH of 6.9-7.1; 100-320 mM sugar component; and 0.4-0.6 mg/mL polysorbate 80.
M713. 15-25 mg/mL EGFR/LGR5 BsAb; 10 mM histidine buffer system; a pH of 6.9-7.1; 100-320 mM sugar component; and 0.4-0.6 mg/mL polysorbate 80.
M714. 20 mg/mL EGFR/LGR5 BsAb; 10 mM histidine buffer system; a pH of 6.9-7.1; 100-320 mM sugar component; and 0.4-0.6 mg/mL polysorbate 80.
M715. 1-150 mg/mL EGFR/LGR5 BsAb; 3-12 mM citrate buffer system; a pH of 6.9-7.1; 100-320 mM sugar component; and 0.4-0.6 mg/mL polysorbate 80.
M716. 15-25 mg/mL EGFR/LGR5 BsAb; 3-12 mM citrate buffer system; a pH of 6.9-7.1; 100-320 mM sugar component; and 0.4-0.6 mg/mL polysorbate 80.
M717. 20 mg/mL EGFR/LGR5 BsAb; 3-12 mM citrate buffer system; a pH of 6.9-7.1; 100-320 mM sugar component; and 0.4-0.6 mg/mL polysorbate 80.
M718. 1-150 mg/mL EGFR/LGR5 BsAb; 10 mM citrate buffer system; a pH of 6.9-7.1; 100-320 mM sugar component; and 0.4-0.6 mg/mL polysorbate 80.
M719. 15-25 mg/mL EGFR/LGR5 BsAb; 10 mM citrate buffer system; a pH of 6.9-7.1; 100-320 mM sugar component; and 0.4-0.6 mg/mL polysorbate 80.
M720. 20 mg/mL EGFR/LGR5 BsAb; 10 mM citrate buffer system; a pH of 6.9-7.1; 100-320 mM sugar component; and 0.4-0.6 mg/mL polysorbate 80.
M721. 1-150 mg/mL EGFR/LGR5 BsAb; 2-70 mM buffer system; a pH of 5.4-7.2; 150-350 mM sucrose; and 0.4-0.6 mg/mL polysorbate 80.
M722. 15-25 mg/mL EGFR/LGR5 BsAb; 2-70 mM buffer system; a pH of 5.4-7.2; 150-350 mM sucrose; and 0.4-0.6 mg/mL polysorbate 80.
M723. 20 mg/mL EGFR/LGR5 BsAb; 2-70 mM buffer system; a pH of 5.4-7.2; 150-350 mM sucrose; and 0.4-0.6 mg/mL polysorbate 80.
M724. 1-150 mg/mL EGFR/LGR5 BsAb; 3-12 mM histidine buffer system; a pH of 5.4-7.2; 150-350 mM sucrose; and 0.4-0.6 mg/mL polysorbate 80.
M725. 15-25 mg/mL EGFR/LGR5 BsAb; 3-12 mM histidine buffer system; a pH of 5.4-7.2; 150-350 mM sucrose; and 0.4-0.6 mg/mL polysorbate 80.
M726. 20 mg/mL EGFR/LGR5 BsAb; 3-12 mM histidine buffer system; a pH of 5.4-7.2; 150-350 mM sucrose; and 0.4-0.6 mg/mL polysorbate 80.
M727. 1-150 mg/mL EGFR/LGR5 BsAb; 5 mM histidine buffer system; a pH of 5.4-7.2; 150-350 mM sucrose; and 0.4-0.6 mg/mL polysorbate 80.
M728. 15-25 mg/mL EGFR/LGR5 BsAb; 5 mM histidine buffer system; a pH of 5.4-7.2; 150-350 mM sucrose; and 0.4-0.6 mg/mL polysorbate 80.
M729. 20 mg/mL EGFR/LGR5 BsAb; 5 mM histidine buffer system; a pH of 5.4-7.2; 150-350 mM sucrose; and 0.4-0.6 mg/mL polysorbate 80.
M730. 1-150 mg/mL EGFR/LGR5 BsAb; 10 mM histidine buffer system; a pH of 5.4-7.2; 150-350 mM sucrose; and 0.4-0.6 mg/mL polysorbate 80.
M731. 15-25 mg/mL EGFR/LGR5 BsAb; 10 mM histidine buffer system; a pH of 5.4-7.2; 150-350 mM sucrose; and 0.4-0.6 mg/mL polysorbate 80.
M732. 20 mg/mL EGFR/LGR5 BsAb; 10 mM histidine buffer system; a pH of 5.4-7.2; 150-350 mM sucrose; and 0.4-0.6 mg/mL polysorbate 80.
M733. 1-150 mg/mL EGFR/LGR5 BsAb; 3-12 mM citrate buffer system; a pH of 5.8-7.8; 150-350 mM sucrose; and 0.4-0.6 mg/mL polysorbate 80.
M734. 15-25 mg/mL EGFR/LGR5 BsAb; 3-12 mM citrate buffer system; a pH of 5.8-7.8; 150-350 mM sucrose; and 0.4-0.6 mg/mL polysorbate 80.
M735. 20 mg/mL EGFR/LGR5 BsAb; 3-12 mM citrate buffer system; a pH of 5.8-7.8; 150-350 mM sucrose; and 0.4-0.6 mg/mL polysorbate 80.
M736. 1-150 mg/mL EGFR/LGR5 BsAb; 10 mM citrate buffer system; a pH of 5.8-7.8; 150-350 mM sucrose; and 0.4-0.6 mg/mL polysorbate 80.
M737. 15-25 mg/mL EGFR/LGR5 BsAb; 10 mM citrate buffer system; a pH of 5.8-7.8; 150-350 mM sucrose; and 0.4-0.6 mg/mL polysorbate 80.
M738. 20 mg/mL EGFR/LGR5 BsAb; 10 mM citrate buffer system; a pH of 5.8-7.8; 150-350 mM sucrose; and 0.4-0.6 mg/mL polysorbate 80.
M739. 1-150 mg/mL EGFR/LGR5 BsAb; 2-70 mM buffer system; a pH of 5.8-6.0; 150-350 mM sucrose; and 0.4-0.6 mg/mL polysorbate 80.
M740. 15-25 mg/mL EGFR/LGR5 BsAb; 2-70 mM buffer system; a pH of 5.8-6.0; 150-350 mM sucrose; and 0.4-0.6 mg/mL polysorbate 80.
M741. 20 mg/mL EGFR/LGR5 BsAb; 2-70 mM buffer system; a pH of 5.8-6.0; 150-350 mM sucrose; and 0.4-0.6 mg/mL polysorbate 80.
M742. 1-150 mg/mL EGFR/LGR5 BsAb; 3-12 mM histidine buffer system; a pH of 5.8-6.0; 150-350 mM sucrose; and 0.4-0.6 mg/mL polysorbate 80.
M743. 15-25 mg/mL EGFR/LGR5 BsAb; 3-12 mM histidine buffer system; a pH of 5.8-6.0; 150-350 mM sucrose; and 0.4-0.6 mg/mL polysorbate 80.
M744. 20 mg/mL EGFR/LGR5 BsAb; 3-12 mM histidine buffer system; a pH of 5.8-6.0; 150-350 mM sucrose; and 0.4-0.6 mg/mL polysorbate 80.
M745. 1-150 mg/mL EGFR/LGR5 BsAb; 5 mM histidine buffer system; a pH of 5.8-6.0; 150-350 mM sucrose; and 0.4-0.6 mg/mL polysorbate 80.
M746. 15-25 mg/mL EGFR/LGR5 BsAb; 5 mM histidine buffer system; a pH of 5.8-6.0; 150-350 mM sucrose; and 0.4-0.6 mg/mL polysorbate 80.
M747. 20 mg/mL EGFR/LGR5 BsAb; 5 mM histidine buffer system; a pH of 5.8-6.0; 150-350 mM sucrose; and 0.4-0.6 mg/mL polysorbate 80.
M748. 1-150 mg/mL EGFR/LGR5 BsAb; 10 mM histidine buffer system; a pH of 5.8-6.0; 150-350 mM sucrose; and 0.4-0.6 mg/mL polysorbate 80.
M749. 15-25 mg/mL EGFR/LGR5 BsAb; 10 mM histidine buffer system; a pH of 5.8-6.0; 150-350 mM sucrose; and 0.4-0.6 mg/mL polysorbate 80.
M750. 20 mg/mL EGFR/LGR5 BsAb; 10 mM histidine buffer system; a pH of 5.8-6.0; 150-350 mM sucrose; and 0.4-0.6 mg/mL polysorbate 80.
M751. 1-150 mg/mL EGFR/LGR5 BsAb; 3-12 mM citrate buffer system; a pH of 5.8-7; 150-350 mM sucrose; and 0.4-0.6 mg/mL polysorbate 80.
M752. 15-25 mg/mL EGFR/LGR5 BsAb; 3-12 mM citrate buffer system; a pH of 5.8-7; 150-350 mM sucrose; and 0.4-0.6 mg/mL polysorbate 80.
M753. 20 mg/mL EGFR/LGR5 BsAb; 3-12 mM citrate buffer system; a pH of 5.8-7; 150-350 mM sucrose; and 0.4-0.6 mg/mL polysorbate 80.
M754. 1-150 mg/mL EGFR/LGR5 BsAb; 10 mM citrate buffer system; a pH of 5.8-7; 150-350 mM sucrose; and 0.4-0.6 mg/mL polysorbate 80.
M755. 15-25 mg/mL EGFR/LGR5 BsAb; 10 mM citrate buffer system; a pH of 5.8-7; 150-350 mM sucrose; and 0.4-0.6 mg/mL polysorbate 80.
M756. 20 mg/mL EGFR/LGR5 BsAb; 10 mM citrate buffer system; a pH of 5.8-7; 150-350 mM sucrose; and 0.4-0.6 mg/mL polysorbate 80.
M757. 1-150 mg/mL EGFR/LGR5 BsAb; 2-70 mM buffer system; a pH of 6.2-6.4; 150-350 mM sucrose; and 0.4-0.6 mg/mL polysorbate 80.
M758. 15-25 mg/mL EGFR/LGR5 BsAb; 2-70 mM buffer system; a pH of 6.2-6.4; 150-350 mM sucrose; and 0.4-0.6 mg/mL polysorbate 80.
M759. 20 mg/mL EGFR/LGR5 BsAb; 2-70 mM buffer system; a pH of 6.2-6.4; 150-350 mM sucrose; and 0.4-0.6 mg/mL polysorbate 80.
M760. 1-150 mg/mL EGFR/LGR5 BsAb; 3-12 mM histidine buffer system; a pH of 6.2-6.4; 150-350 mM sucrose; and 0.4-0.6 mg/mL polysorbate 80.
M761. 15-25 mg/mL EGFR/LGR5 BsAb; 3-12 mM histidine buffer system; a pH of 6.2-6.4; 150-350 mM sucrose; and 0.4-0.6 mg/mL polysorbate 80.
M762. 20 mg/mL EGFR/LGR5 BsAb; 3-12 mM histidine buffer system; a pH of 6.2-6.4; 150-350 mM sucrose; and 0.4-0.6 mg/mL polysorbate 80.
M763. 1-150 mg/mL EGFR/LGR5 BsAb; 5 mM histidine buffer system; a pH of 6.2-6.4; 150-350 mM sucrose; and 0.4-0.6 mg/mL polysorbate 80.
M764. 15-25 mg/mL EGFR/LGR5 BsAb; 5 mM histidine buffer system; a pH of 6.2-6.4; 150-350 mM sucrose; and 0.4-0.6 mg/mL polysorbate 80.
M765. 20 mg/mL EGFR/LGR5 BsAb; 5 mM histidine buffer system; a pH of 6.2-6.4; 150-350 mM sucrose; and 0.4-0.6 mg/mL polysorbate 80.
M766. 1-150 mg/mL EGFR/LGR5 BsAb; 10 mM histidine buffer system; a pH of 6.2-6.4; 150-350 mM sucrose; and 0.4-0.6 mg/mL polysorbate 80.
M767. 15-25 mg/mL EGFR/LGR5 BsAb; 10 mM histidine buffer system; a pH of 6.2-6.4; 150-350 mM sucrose; and 0.4-0.6 mg/mL polysorbate 80.
M768. 20 mg/mL EGFR/LGR5 BsAb; 10 mM histidine buffer system; a pH of 6.2-6.4; 150-350 mM sucrose; and 0.4-0.6 mg/mL polysorbate 80.
M769. 1-150 mg/mL EGFR/LGR5 BsAb; 3-12 mM citrate buffer system; a pH of 6.2-7.4; 150-350 mM sucrose; and 0.4-0.6 mg/mL polysorbate 80.
M770. 15-25 mg/mL EGFR/LGR5 BsAb; 3-12 mM citrate buffer system; a pH of 6.2-7.4; 150-350 mM sucrose; and 0.4-0.6 mg/mL polysorbate 80.
M771. 20 mg/mL EGFR/LGR5 BsAb; 3-12 mM citrate buffer system; a pH of 6.2-7.4; 150-350 mM sucrose; and 0.4-0.6 mg/mL polysorbate 80.
M772. 1-150 mg/mL EGFR/LGR5 BsAb; 10 mM citrate buffer system; a pH of 6.2-7.4; 150-350 mM sucrose; and 0.4-0.6 mg/mL polysorbate 80.
M773. 15-25 mg/mL EGFR/LGR5 BsAb; 10 mM citrate buffer system; a pH of 6.2-7.4; 150-350 mM sucrose; and 0.4-0.6 mg/mL polysorbate 80.
M774. 20 mg/mL EGFR/LGR5 BsAb; 10 mM citrate buffer system; a pH of 6.2-7.4; 150-350 mM sucrose; and 0.4-0.6 mg/mL polysorbate 80.
M775. 1-150 mg/mL EGFR/LGR5 BsAb; 2-70 mM buffer system; a pH of 6.9-7.1; 150-350 mM sucrose; and 0.4-0.6 mg/mL polysorbate 80.
M776. 15-25 mg/mL EGFR/LGR5 BsAb; 2-70 mM buffer system; a pH of 6.9-7.1; 150-350 mM sucrose; and 0.4-0.6 mg/mL polysorbate 80.
M777. 20 mg/mL EGFR/LGR5 BsAb; 2-70 mM buffer system; a pH of 6.9-7.1; 150-350 mM sucrose; and 0.4-0.6 mg/mL polysorbate 80.
M778. 1-150 mg/mL EGFR/LGR5 BsAb; 3-12 mM histidine buffer system; a pH of 6.9-7.1; 150-350 mM sucrose; and 0.4-0.6 mg/mL polysorbate 80.
M779. 15-25 mg/mL EGFR/LGR5 BsAb; 3-12 mM histidine buffer system; a pH of 6.9-7.1; 150-350 mM sucrose; and 0.4-0.6 mg/mL polysorbate 80.
M780. 20 mg/mL EGFR/LGR5 BsAb; 3-12 mM histidine buffer system; a pH of 6.9-7.1; 150-350 mM sucrose; and 0.4-0.6 mg/mL polysorbate 80.
M781. 1-150 mg/mL EGFR/LGR5 BsAb; 5 mM histidine buffer system; a pH of 6.9-7.1; 150-350 mM sucrose; and 0.4-0.6 mg/mL polysorbate 80.
M782. 15-25 mg/mL EGFR/LGR5 BsAb; 5 mM histidine buffer system; a pH of 6.9-7.1; 150-350 mM sucrose; and 0.4-0.6 mg/mL polysorbate 80.
M783. 20 mg/mL EGFR/LGR5 BsAb; 5 mM histidine buffer system; a pH of 6.9-7.1; 150-350 mM sucrose; and 0.4-0.6 mg/mL polysorbate 80.
M784. 1-150 mg/mL EGFR/LGR5 BsAb; 10 mM histidine buffer system; a pH of 6.9-7.1; 150-350 mM sucrose; and 0.4-0.6 mg/mL polysorbate 80.
M785. 15-25 mg/mL EGFR/LGR5 BsAb; 10 mM histidine buffer system; a pH of 6.9-7.1; 150-350 mM sucrose; and 0.4-0.6 mg/mL polysorbate 80.
M786. 20 mg/mL EGFR/LGR5 BsAb; 10 mM histidine buffer system; a pH of 6.9-7.1; 150-350 mM sucrose; and 0.4-0.6 mg/mL polysorbate 80.
M787. 1-150 mg/mL EGFR/LGR5 BsAb; 3-12 mM citrate buffer system; a pH of 6.9-7.1; 150-350 mM sucrose; and 0.4-0.6 mg/mL polysorbate 80.
M788. 15-25 mg/mL EGFR/LGR5 BsAb; 3-12 mM citrate buffer system; a pH of 6.9-7.1; 150-350 mM sucrose; and 0.4-0.6 mg/mL polysorbate 80.
M789. 20 mg/mL EGFR/LGR5 BsAb; 3-12 mM citrate buffer system; a pH of 6.9-7.1; 150-350 mM sucrose; and 0.4-0.6 mg/mL polysorbate 80.
M790. 1-150 mg/mL EGFR/LGR5 BsAb; 10 mM citrate buffer system; a pH of 6.9-7.1; 150-350 mM sucrose; and 0.4-0.6 mg/mL polysorbate 80.
M791. 15-25 mg/mL EGFR/LGR5 BsAb; 10 mM citrate buffer system; a pH of 6.9-7.1; 150-350 mM sucrose; and 0.4-0.6 mg/mL polysorbate 80.
M792. 20 mg/mL EGFR/LGR5 BsAb; 10 mM citrate buffer system; a pH of 6.9-7.1; 150-350 mM sucrose; and 0.4-0.6 mg/mL polysorbate 80.
M793. 1-150 mg/mL EGFR/LGR5 BsAb; 2-70 mM buffer system; a pH of 5.4-7.2; 275-295 mM sucrose; and 0.4-0.6 mg/mL polysorbate 80.
M794. 15-25 mg/mL EGFR/LGR5 BsAb; 2-70 mM buffer system; a pH of 5.4-7.2; 275-295 mM sucrose; and 0.4-0.6 mg/mL polysorbate 80.
M795. 20 mg/mL EGFR/LGR5 BsAb; 2-70 mM buffer system; a pH of 5.4-7.2; 275-295 mM sucrose; and 0.4-0.6 mg/mL polysorbate 80.
M796. 1-150 mg/mL EGFR/LGR5 BsAb; 3-12 mM histidine buffer system; a pH of 5.4-7.2; 275-295 mM sucrose; and 0.4-0.6 mg/mL polysorbate 80.
M797. 15-25 mg/mL EGFR/LGR5 BsAb; 3-12 mM histidine buffer system; a pH of 5.4-7.2; 275-295 mM sucrose; and 0.4-0.6 mg/mL polysorbate 80.
M798. 20 mg/mL EGFR/LGR5 BsAb; 3-12 mM histidine buffer system; a pH of 5.4-7.2; 275-295 mM sucrose; and 0.4-0.6 mg/mL polysorbate 80.
M799. 1-150 mg/mL EGFR/LGR5 BsAb; 5 mM histidine buffer system; a pH of 5.4-7.2; 275-295 mM sucrose; and 0.4-0.6 mg/mL polysorbate 80.
M800. 15-25 mg/mL EGFR/LGR5 BsAb; 5 mM histidine buffer system; a pH of 5.4-7.2; 275-295 mM sucrose; and 0.4-0.6 mg/mL polysorbate 80.
M801. 20 mg/mL EGFR/LGR5 BsAb; 5 mM histidine buffer system; a pH of 5.4-7.2; 275-295 mM sucrose; and 0.4-0.6 mg/mL polysorbate 80.
M802. 1-150 mg/mL EGFR/LGR5 BsAb; 10 mM histidine buffer system; a pH of 5.4-7.2; 275-295 mM sucrose; and 0.4-0.6 mg/mL polysorbate 80.
M803. 15-25 mg/mL EGFR/LGR5 BsAb; 10 mM histidine buffer system; a pH of 5.4-7.2; 275-295 mM sucrose; and 0.4-0.6 mg/mL polysorbate 80.
M804. 20 mg/mL EGFR/LGR5 BsAb; 10 mM histidine buffer system; a pH of 5.4-7.2; 275-295 mM sucrose; and 0.4-0.6 mg/mL polysorbate 80.
M805. 1-150 mg/mL EGFR/LGR5 BsAb; 3-12 mM citrate buffer system; a pH of 5.8-7.8; 275-295 mM sucrose; and 0.4-0.6 mg/mL polysorbate 80.
M806. 15-25 mg/mL EGFR/LGR5 BsAb; 3-12 mM citrate buffer system; a pH of 5.8-7.8; 275-295 mM sucrose; and 0.4-0.6 mg/mL polysorbate 80.
M807. 20 mg/mL EGFR/LGR5 BsAb; 3-12 mM citrate buffer system; a pH of 5.8-7.8; 275-295 mM sucrose; and 0.4-0.6 mg/mL polysorbate 80.
M808. 1-150 mg/mL EGFR/LGR5 BsAb; 10 mM citrate buffer system; a pH of 5.8-7.8; 275-295 mM sucrose; and 0.4-0.6 mg/mL polysorbate 80.
M809. 15-25 mg/mL EGFR/LGR5 BsAb; 10 mM citrate buffer system; a pH of 5.8-7.8; 275-295 mM sucrose; and 0.4-0.6 mg/mL polysorbate 80.
M810. 20 mg/mL EGFR/LGR5 BsAb; 10 mM citrate buffer system; a pH of 5.8-7.8; 275-295 mM sucrose; and 0.4-0.6 mg/mL polysorbate 80.
M811. 1-150 mg/mL EGFR/LGR5 BsAb; 2-70 mM buffer system; a pH of 5.8-6.0; 275-295 mM sucrose; and 0.4-0.6 mg/mL polysorbate 80.
M812. 15-25 mg/mL EGFR/LGR5 BsAb; 2-70 mM buffer system; a pH of 5.8-6.0; 275-295 mM sucrose; and 0.4-0.6 mg/mL polysorbate 80.
M813. 20 mg/mL EGFR/LGR5 BsAb; 2-70 mM buffer system; a pH of 5.8-6.0; 275-295 mM sucrose; and 0.4-0.6 mg/mL polysorbate 80.
M814. 1-150 mg/mL EGFR/LGR5 BsAb; 3-12 mM histidine buffer system; a pH of 5.8-6.0; 275-295 mM sucrose; and 0.4-0.6 mg/mL polysorbate 80.
M815. 15-25 mg/mL EGFR/LGR5 BsAb; 3-12 mM histidine buffer system; a pH of 5.8-6.0; 275-295 mM sucrose; and 0.4-0.6 mg/mL polysorbate 80.
M816. 20 mg/mL EGFR/LGR5 BsAb; 3-12 mM histidine buffer system; a pH of 5.8-6.0; 275-295 mM sucrose; and 0.4-0.6 mg/mL polysorbate 80.
M817. 1-150 mg/mL EGFR/LGR5 BsAb; 5 mM histidine buffer system; a pH of 5.8-6.0; 275-295 mM sucrose; and 0.4-0.6 mg/mL polysorbate 80.
M818. 15-25 mg/mL EGFR/LGR5 BsAb; 5 mM histidine buffer system; a pH of 5.8-6.0; 275-295 mM sucrose; and 0.4-0.6 mg/mL polysorbate 80.
M819. 20 mg/mL EGFR/LGR5 BsAb; 5 mM histidine buffer system; a pH of 5.8-6.0; 275-295 mM sucrose; and 0.4-0.6 mg/mL polysorbate 80.
M820. 1-150 mg/mL EGFR/LGR5 BsAb; 10 mM histidine buffer system; a pH of 5.8-6.0; 275-295 mM sucrose; and 0.4-0.6 mg/mL polysorbate 80.
M821. 15-25 mg/mL EGFR/LGR5 BsAb; 10 mM histidine buffer system; a pH of 5.8-6.0; 275-295 mM sucrose; and 0.4-0.6 mg/mL polysorbate 80.
M822. 20 mg/mL EGFR/LGR5 BsAb; 10 mM histidine buffer system; a pH of 5.8-6.0; 275-295 mM sucrose; and 0.4-0.6 mg/mL polysorbate 80.
M823. 1-150 mg/mL EGFR/LGR5 BsAb; 3-12 mM citrate buffer system; a pH of 5.8-7; 275-295 mM sucrose; and 0.4-0.6 mg/mL polysorbate 80.
M824. 15-25 mg/mL EGFR/LGR5 BsAb; 3-12 mM citrate buffer system; a pH of 5.8-7; 275-295 mM sucrose; and 0.4-0.6 mg/mL polysorbate 80.
M825. 20 mg/mL EGFR/LGR5 BsAb; 3-12 mM citrate buffer system; a pH of 5.8-7; 275-295 mM sucrose; and 0.4-0.6 mg/mL polysorbate 80.
M826. 1-150 mg/mL EGFR/LGR5 BsAb; 10 mM citrate buffer system; a pH of 5.8-7; 275-295 mM sucrose; and 0.4-0.6 mg/mL polysorbate 80.
M827. 15-25 mg/mL EGFR/LGR5 BsAb; 10 mM citrate buffer system; a pH of 5.8-7; 275-295 mM sucrose; and 0.4-0.6 mg/mL polysorbate 80.
M828. 20 mg/mL EGFR/LGR5 BsAb; 10 mM citrate buffer system; a pH of 5.8-7; 275-295 mM sucrose; and 0.4-0.6 mg/mL polysorbate 80.
M829. 1-150 mg/mL EGFR/LGR5 BsAb; 2-70 mM buffer system; a pH of 6.2-6.4; 275-295 mM sucrose; and 0.4-0.6 mg/mL polysorbate 80.
M830. 15-25 mg/mL EGFR/LGR5 BsAb; 2-70 mM buffer system; a pH of 6.2-6.4; 275-295 mM sucrose; and 0.4-0.6 mg/mL polysorbate 80.
M831. 20 mg/mL EGFR/LGR5 BsAb; 2-70 mM buffer system; a pH of 6.2-6.4; 275-295 mM sucrose; and 0.4-0.6 mg/mL polysorbate 80.
M832. 1-150 mg/mL EGFR/LGR5 BsAb; 3-12 mM histidine buffer system; a pH of 6.2-6.4; 275-295 mM sucrose; and 0.4-0.6 mg/mL polysorbate 80.
M833. 15-25 mg/mL EGFR/LGR5 BsAb; 3-12 mM histidine buffer system; a pH of 6.2-6.4; 275-295 mM sucrose; and 0.4-0.6 mg/mL polysorbate 80.
M834. 20 mg/mL EGFR/LGR5 BsAb; 3-12 mM histidine buffer system; a pH of 6.2-6.4; 275-295 mM sucrose; and 0.4-0.6 mg/mL polysorbate 80.
M835. 1-150 mg/mL EGFR/LGR5 BsAb; 5 mM histidine buffer system; a pH of 6.2-6.4; 275-295 mM sucrose; and 0.4-0.6 mg/mL polysorbate 80.
M836. 15-25 mg/mL EGFR/LGR5 BsAb; 5 mM histidine buffer system; a pH of 6.2-6.4; 275-295 mM sucrose; and 0.4-0.6 mg/mL polysorbate 80.
M837. 20 mg/mL EGFR/LGR5 BsAb; 5 mM histidine buffer system; a pH of 6.2-6.4; 275-295 mM sucrose; and 0.4-0.6 mg/mL polysorbate 80.
M838. 1-150 mg/mL EGFR/LGR5 BsAb; 10 mM histidine buffer system; a pH of 6.2-6.4; 275-295 mM sucrose; and 0.4-0.6 mg/mL polysorbate 80.
M839. 15-25 mg/mL EGFR/LGR5 BsAb; 10 mM histidine buffer system; a pH of 6.2-6.4; 275-295 mM sucrose; and 0.4-0.6 mg/mL polysorbate 80.
M840. 20 mg/mL EGFR/LGR5 BsAb; 10 mM histidine buffer system; a pH of 6.2-6.4; 275-295 mM sucrose; and 0.4-0.6 mg/mL polysorbate 80.
M841. 1-150 mg/mL EGFR/LGR5 BsAb; 3-12 mM citrate buffer system; a pH of 6.2-7.4; 275-295 mM sucrose; and 0.4-0.6 mg/mL polysorbate 80.
M842. 15-25 mg/mL EGFR/LGR5 BsAb; 3-12 mM citrate buffer system; a pH of 6.2-7.4; 275-295 mM sucrose; and 0.4-0.6 mg/mL polysorbate 80.
M843. 20 mg/mL EGFR/LGR5 BsAb; 3-12 mM citrate buffer system; a pH of 6.2-7.4; 275-295 mM sucrose; and 0.4-0.6 mg/mL polysorbate 80.
M844. 1-150 mg/mL EGFR/LGR5 BsAb; 10 mM citrate buffer system; a pH of 6.2-7.4; 275-295 mM sucrose; and 0.4-0.6 mg/mL polysorbate 80.
M845. 15-25 mg/mL EGFR/LGR5 BsAb; 10 mM citrate buffer system; a pH of 6.2-7.4; 275-295 mM sucrose; and 0.4-0.6 mg/mL polysorbate 80.
M846. 20 mg/mL EGFR/LGR5 BsAb; 10 mM citrate buffer system; a pH of 6.2-7.4; 275-295 mM sucrose; and 0.4-0.6 mg/mL polysorbate 80.
M847. 1-150 mg/mL EGFR/LGR5 BsAb; 2-70 mM buffer system; a pH of 6.9-7.1; 275-295 mM sucrose; and 0.4-0.6 mg/mL polysorbate 80.
M848. 15-25 mg/mL EGFR/LGR5 BsAb; 2-70 mM buffer system; a pH of 6.9-7.1; 275-295 mM sucrose; and 0.4-0.6 mg/mL polysorbate 80.
M849. 20 mg/mL EGFR/LGR5 BsAb; 2-70 mM buffer system; a pH of 6.9-7.1; 275-295 mM sucrose; and 0.4-0.6 mg/mL polysorbate 80.
M850. 1-150 mg/mL EGFR/LGR5 BsAb; 3-12 mM histidine buffer system; a pH of 6.9-7.1; 275-295 mM sucrose; and 0.4-0.6 mg/mL polysorbate 80.
M851. 15-25 mg/mL EGFR/LGR5 BsAb; 3-12 mM histidine buffer system; a pH of 6.9-7.1; 275-295 mM sucrose; and 0.4-0.6 mg/mL polysorbate 80.
M852. 20 mg/mL EGFR/LGR5 BsAb; 3-12 mM histidine buffer system; a pH of 6.9-7.1; 275-295 mM sucrose; and 0.4-0.6 mg/mL polysorbate 80.
M853. 1-150 mg/mL EGFR/LGR5 BsAb; 5 mM histidine buffer system; a pH of 6.9-7.1; 275-295 mM sucrose; and 0.4-0.6 mg/mL polysorbate 80.
M854. 15-25 mg/mL EGFR/LGR5 BsAb; 5 mM histidine buffer system; a pH of 6.9-7.1; 275-295 mM sucrose; and 0.4-0.6 mg/mL polysorbate 80.
M855. 20 mg/mL EGFR/LGR5 BsAb; 5 mM histidine buffer system; a pH of 6.9-7.1; 275-295 mM sucrose; and 0.4-0.6 mg/mL polysorbate 80.
M856. 1-150 mg/mL EGFR/LGR5 BsAb; 10 mM histidine buffer system; a pH of 6.9-7.1; 275-295 mM sucrose; and 0.4-0.6 mg/mL polysorbate 80.
M857. 15-25 mg/mL EGFR/LGR5 BsAb; 10 mM histidine buffer system; a pH of 6.9-7.1; 275-295 mM sucrose; and 0.4-0.6 mg/mL polysorbate 80.
M858. 20 mg/mL EGFR/LGR5 BsAb; 10 mM histidine buffer system; a pH of 6.9-7.1; 275-295 mM sucrose; and 0.4-0.6 mg/mL polysorbate 80.
M859. 1-150 mg/mL EGFR/LGR5 BsAb; 3-12 mM citrate buffer system; a pH of 6.9-7.1; 275-295 mM sucrose; and 0.4-0.6 mg/mL polysorbate 80.
M860. 15-25 mg/mL EGFR/LGR5 BsAb; 3-12 mM citrate buffer system; a pH of 6.9-7.1; 275-295 mM sucrose; and 0.4-0.6 mg/mL polysorbate 80.
M861. 20 mg/mL EGFR/LGR5 BsAb; 3-12 mM citrate buffer system; a pH of 6.9-7.1; 275-295 mM sucrose; and 0.4-0.6 mg/mL polysorbate 80.
M862. 1-150 mg/mL EGFR/LGR5 BsAb; 10 mM citrate buffer system; a pH of 6.9-7.1; 275-295 mM sucrose; and 0.4-0.6 mg/mL polysorbate 80.
M863. 15-25 mg/mL EGFR/LGR5 BsAb; 10 mM citrate buffer system; a pH of 6.9-7.1; 275-295 mM sucrose; and 0.4-0.6 mg/mL polysorbate 80.
M864. 20 mg/mL EGFR/LGR5 BsAb; 10 mM citrate buffer system; a pH of 6.9-7.1; 275-295 mM sucrose; and 0.4-0.6 mg/mL polysorbate 80.
M865. 1-150 mg/mL EGFR/LGR5 BsAb; 2-70 mM buffer system; a pH of 5.4-7.2; 100-320 mM sugar component; and 0.9-1.1 mg/mL polysorbate 80.
M866. 15-25 mg/mL EGFR/LGR5 BsAb; 2-70 mM buffer system; a pH of 5.4-7.2; 100-320 mM sugar component; and 0.9-1.1 mg/mL polysorbate 80.
M867. 20 mg/mL EGFR/LGR5 BsAb; 2-70 mM buffer system; a pH of 5.4-7.2; 100-320 mM sugar component; and 0.9-1.1 mg/mL polysorbate 80.
M868. 1-150 mg/mL EGFR/LGR5 BsAb; 3-12 mM histidine buffer system; a pH of 5.4-7.2; 100-320 mM sugar component; and 0.9-1.1 mg/mL polysorbate 80.
M869. 15-25 mg/mL EGFR/LGR5 BsAb; 3-12 mM histidine buffer system; a pH of 5.4-7.2; 100-320 mM sugar component; and 0.9-1.1 mg/mL polysorbate 80.
M870. 20 mg/mL EGFR/LGR5 BsAb; 3-12 mM histidine buffer system; a pH of 5.4-7.2; 100-320 mM sugar component; and 0.9-1.1 mg/mL polysorbate 80.
M871. 1-150 mg/mL EGFR/LGR5 BsAb; 5 mM histidine buffer system; a pH of 5.4-7.2; 100-320 mM sugar component; and 0.9-1.1 mg/mL polysorbate 80.
M872. 15-25 mg/mL EGFR/LGR5 BsAb; 5 mM histidine buffer system; a pH of 5.4-7.2; 100-320 mM sugar component; and 0.9-1.1 mg/mL polysorbate 80.
M873. 20 mg/mL EGFR/LGR5 BsAb; 5 mM histidine buffer system; a pH of 5.4-7.2; 100-320 mM sugar component; and 0.9-1.1 mg/mL polysorbate 80.
M874. 1-150 mg/mL EGFR/LGR5 BsAb; 10 mM histidine buffer system; a pH of 5.4-7.2; 100-320 mM sugar component; and 0.9-1.1 mg/mL polysorbate 80.
M875. 15-25 mg/mL EGFR/LGR5 BsAb; 10 mM histidine buffer system; a pH of 5.4-7.2; 100-320 mM sugar component; and 0.9-1.1 mg/mL polysorbate 80.
M876. 20 mg/mL EGFR/LGR5 BsAb; 10 mM histidine buffer system; a pH of 5.4-7.2; 100-320 mM sugar component; and 0.9-1.1 mg/mL polysorbate 80.
M877. 1-150 mg/mL EGFR/LGR5 BsAb; 3-12 mM citrate buffer system; a pH of 5.8-7.8; 100-320 mM sugar component; and 0.9-1.1 mg/mL polysorbate 80.
M878. 15-25 mg/mL EGFR/LGR5 BsAb; 3-12 mM citrate buffer system; a pH of 5.8-7.8; 100-320 mM sugar component; and 0.9-1.1 mg/mL polysorbate 80.
M879. 20 mg/mL EGFR/LGR5 BsAb; 3-12 mM citrate buffer system; a pH of 5.8-7.8; 100-320 mM sugar component; and 0.9-1.1 mg/mL polysorbate 80.
M880. 1-150 mg/mL EGFR/LGR5 BsAb; 10 mM citrate buffer system; a pH of 5.8-7.8; 100-320 mM sugar component; and 0.9-1.1 mg/mL polysorbate 80.
M881. 15-25 mg/mL EGFR/LGR5 BsAb; 10 mM citrate buffer system; a pH of 5.8-7.8; 100-320 mM sugar component; and 0.9-1.1 mg/mL polysorbate 80.
M882. 20 mg/mL EGFR/LGR5 BsAb; 10 mM citrate buffer system; a pH of 5.8-7.8; 100-320 mM sugar component; and 0.9-1.1 mg/mL polysorbate 80.
M883. 1-150 mg/mL EGFR/LGR5 BsAb; 2-70 mM buffer system; a pH of 5.8-6.0; 100-320 mM sugar component; and 0.9-1.1 mg/mL polysorbate 80.
M884. 15-25 mg/mL EGFR/LGR5 BsAb; 2-70 mM buffer system; a pH of 5.8-6.0; 100-320 mM sugar component; and 0.9-1.1 mg/mL polysorbate 80.
M885. 20 mg/mL EGFR/LGR5 BsAb; 2-70 mM buffer system; a pH of 5.8-6.0; 100-320 mM sugar component; and 0.9-1.1 mg/mL polysorbate 80.
M886. 1-150 mg/mL EGFR/LGR5 BsAb; 3-12 mM histidine buffer system; a pH of 5.8-6.0; 100-320 mM sugar component; and 0.9-1.1 mg/mL polysorbate 80.
M887. 15-25 mg/mL EGFR/LGR5 BsAb; 3-12 mM histidine buffer system; a pH of 5.8-6.0; 100-320 mM sugar component; and 0.9-1.1 mg/mL polysorbate 80.
M888. 20 mg/mL EGFR/LGR5 BsAb; 3-12 mM histidine buffer system; a pH of 5.8-6.0; 100-320 mM sugar component; and 0.9-1.1 mg/mL polysorbate 80.
M889. 1-150 mg/mL EGFR/LGR5 BsAb; 5 mM histidine buffer system; a pH of 5.8-6.0; 100-320 mM sugar component; and 0.9-1.1 mg/mL polysorbate 80.
M890. 15-25 mg/mL EGFR/LGR5 BsAb; 5 mM histidine buffer system; a pH of 5.8-6.0; 100-320 mM sugar component; and 0.9-1.1 mg/mL polysorbate 80.
M891. 20 mg/mL EGFR/LGR5 BsAb; 5 mM histidine buffer system; a pH of 5.8-6.0; 100-320 mM sugar component; and 0.9-1.1 mg/mL polysorbate 80.
M892. 1-150 mg/mL EGFR/LGR5 BsAb; 10 mM histidine buffer system; a pH of 5.8-6.0; 100-320 mM sugar component; and 0.9-1.1 mg/mL polysorbate 80.
M893. 15-25 mg/mL EGFR/LGR5 BsAb; 10 mM histidine buffer system; a pH of 5.8-6.0; 100-320 mM sugar component; and 0.9-1.1 mg/mL polysorbate 80.
M894. 20 mg/mL EGFR/LGR5 BsAb; 10 mM histidine buffer system; a pH of 5.8-6.0; 100-320 mM sugar component; and 0.9-1.1 mg/mL polysorbate 80.
M895. 1-150 mg/mL EGFR/LGR5 BsAb; 3-12 mM citrate buffer system; a pH of 5.8-7; 100-320 mM sugar component; and 0.9-1.1 mg/mL polysorbate 80.
M896. 15-25 mg/mL EGFR/LGR5 BsAb; 3-12 mM citrate buffer system; a pH of 5.8-7; 100-320 mM sugar component; and 0.9-1.1 mg/mL polysorbate 80.
M897. 20 mg/mL EGFR/LGR5 BsAb; 3-12 mM citrate buffer system; a pH of 5.8-7; 100-320 mM sugar component; and 0.9-1.1 mg/mL polysorbate 80.
M898. 1-150 mg/mL EGFR/LGR5 BsAb; 10 mM citrate buffer system; a pH of 5.8-7; 100-320 mM sugar component; and 0.9-1.1 mg/mL polysorbate 80.
M899. 15-25 mg/mL EGFR/LGR5 BsAb; 10 mM citrate buffer system; a pH of 5.8-7; 100-320 mM sugar component; and 0.9-1.1 mg/mL polysorbate 80.
M900. 20 mg/mL EGFR/LGR5 BsAb; 10 mM citrate buffer system; a pH of 5.8-7; 100-320 mM sugar component; and 0.9-1.1 mg/mL polysorbate 80.
M901. 1-150 mg/mL EGFR/LGR5 BsAb; 2-70 mM buffer system; a pH of 6.2-6.4; 100-320 mM sugar component; and 0.9-1.1 mg/mL polysorbate 80.
M902. 15-25 mg/mL EGFR/LGR5 BsAb; 2-70 mM buffer system; a pH of 6.2-6.4; 100-320 mM sugar component; and 0.9-1.1 mg/mL polysorbate 80.
M903. 20 mg/mL EGFR/LGR5 BsAb; 2-70 mM buffer system; a pH of 6.2-6.4; 100-320 mM sugar component; and 0.9-1.1 mg/mL polysorbate 80.
M904. 1-150 mg/mL EGFR/LGR5 BsAb; 3-12 mM histidine buffer system; a pH of 6.2-6.4; 100-320 mM sugar component; and 0.9-1.1 mg/mL polysorbate 80.
M905. 15-25 mg/mL EGFR/LGR5 BsAb; 3-12 mM histidine buffer system; a pH of 6.2-6.4; 100-320 mM sugar component; and 0.9-1.1 mg/mL polysorbate 80.
M906. 20 mg/mL EGFR/LGR5 BsAb; 3-12 mM histidine buffer system; a pH of 6.2-6.4; 100-320 mM sugar component; and 0.9-1.1 mg/mL polysorbate 80.
M907. 1-150 mg/mL EGFR/LGR5 BsAb; 5 mM histidine buffer system; a pH of 6.2-6.4; 100-320 mM sugar component; and 0.9-1.1 mg/mL polysorbate 80.
M908. 15-25 mg/mL EGFR/LGR5 BsAb; 5 mM histidine buffer system; a pH of 6.2-6.4; 100-320 mM sugar component; and 0.9-1.1 mg/mL polysorbate 80.
M909. 20 mg/mL EGFR/LGR5 BsAb; 5 mM histidine buffer system; a pH of 6.2-6.4; 100-320 mM sugar component; and 0.9-1.1 mg/mL polysorbate 80.
M910. 1-150 mg/mL EGFR/LGR5 BsAb; 10 mM histidine buffer system; a pH of 6.2-6.4; 100-320 mM sugar component; and 0.9-1.1 mg/mL polysorbate 80.
M911. 15-25 mg/mL EGFR/LGR5 BsAb; 10 mM histidine buffer system; a pH of 6.2-6.4; 100-320 mM sugar component; and 0.9-1.1 mg/mL polysorbate 80.
M912. 20 mg/mL EGFR/LGR5 BsAb; 10 mM histidine buffer system; a pH of 6.2-6.4; 100-320 mM sugar component; and 0.9-1.1 mg/mL polysorbate 80.
M913. 1-150 mg/mL EGFR/LGR5 BsAb; 3-12 mM citrate buffer system; a pH of 6.2-7.4; 100-320 mM sugar component; and 0.9-1.1 mg/mL polysorbate 80.
M914. 15-25 mg/mL EGFR/LGR5 BsAb; 3-12 mM citrate buffer system; a pH of 6.2-7.4; 100-320 mM sugar component; and 0.9-1.1 mg/mL polysorbate 80.
M915. 20 mg/mL EGFR/LGR5 BsAb; 3-12 mM citrate buffer system; a pH of 6.2-7.4; 100-320 mM sugar component; and 0.9-1.1 mg/mL polysorbate 80.
M916. 1-150 mg/mL EGFR/LGR5 BsAb; 10 mM citrate buffer system; a pH of 6.2-7.4; 100-320 mM sugar component; and 0.9-1.1 mg/mL polysorbate 80.
M917. 15-25 mg/mL EGFR/LGR5 BsAb; 10 mM citrate buffer system; a pH of 6.2-7.4; 100-320 mM sugar component; and 0.9-1.1 mg/mL polysorbate 80.
M918. 20 mg/mL EGFR/LGR5 BsAb; 10 mM citrate buffer system; a pH of 6.2-7.4; 100-320 mM sugar component; and 0.9-1.1 mg/mL polysorbate 80.
M919. 1-150 mg/mL EGFR/LGR5 BsAb; 2-70 mM buffer system; a pH of 6.9-7.1; 100-320 mM sugar component; and 0.9-1.1 mg/mL polysorbate 80.
M920. 15-25 mg/mL EGFR/LGR5 BsAb; 2-70 mM buffer system; a pH of 6.9-7.1; 100-320 mM sugar component; and 0.9-1.1 mg/mL polysorbate 80.
M921. 20 mg/mL EGFR/LGR5 BsAb; 2-70 mM buffer system; a pH of 6.9-7.1; 100-320 mM sugar component; and 0.9-1.1 mg/mL polysorbate 80.
M922. 1-150 mg/mL EGFR/LGR5 BsAb; 3-12 mM histidine buffer system; a pH of 6.9-7.1; 100-320 mM sugar component; and 0.9-1.1 mg/mL polysorbate 80.
M923. 15-25 mg/mL EGFR/LGR5 BsAb; 3-12 mM histidine buffer system; a pH of 6.9-7.1; 100-320 mM sugar component; and 0.9-1.1 mg/mL polysorbate 80.
M924. 20 mg/mL EGFR/LGR5 BsAb; 3-12 mM histidine buffer system; a pH of 6.9-7.1; 100-320 mM sugar component; and 0.9-1.1 mg/mL polysorbate 80.
M925. 1-150 mg/mL EGFR/LGR5 BsAb; 5 mM histidine buffer system; a pH of 6.9-7.1; 100-320 mM sugar component; and 0.9-1.1 mg/mL polysorbate 80.
M926. 15-25 mg/mL EGFR/LGR5 BsAb; 5 mM histidine buffer system; a pH of 6.9-7.1; 100-320 mM sugar component; and 0.9-1.1 mg/mL polysorbate 80.
M927. 20 mg/mL EGFR/LGR5 BsAb; 5 mM histidine buffer system; a pH of 6.9-7.1; 100-320 mM sugar component; and 0.9-1.1 mg/mL polysorbate 80.
M928. 1-150 mg/mL EGFR/LGR5 BsAb; 10 mM histidine buffer system; a pH of 6.9-7.1; 100-320 mM sugar component; and 0.9-1.1 mg/mL polysorbate 80.
M929. 15-25 mg/mL EGFR/LGR5 BsAb; 10 mM histidine buffer system; a pH of 6.9-7.1; 100-320 mM sugar component; and 0.9-1.1 mg/mL polysorbate 80.
M930. 20 mg/mL EGFR/LGR5 BsAb; 10 mM histidine buffer system; a pH of 6.9-7.1; 100-320 mM sugar component; and 0.9-1.1 mg/mL polysorbate 80.
M931. 1-150 mg/mL EGFR/LGR5 BsAb; 3-12 mM citrate buffer system; a pH of 6.9-7.1; 100-320 mM sugar component; and 0.9-1.1 mg/mL polysorbate 80.
M932. 15-25 mg/mL EGFR/LGR5 BsAb; 3-12 mM citrate buffer system; a pH of 6.9-7.1; 100-320 mM sugar component; and 0.9-1.1 mg/mL polysorbate 80.
M933. 20 mg/mL EGFR/LGR5 BsAb; 3-12 mM citrate buffer system; a pH of 6.9-7.1; 100-320 mM sugar component; and 0.9-1.1 mg/mL polysorbate 80.
M934. 1-150 mg/mL EGFR/LGR5 BsAb; 10 mM citrate buffer system; a pH of 6.9-7.1; 100-320 mM sugar component; and 0.9-1.1 mg/mL polysorbate 80.
M935. 15-25 mg/mL EGFR/LGR5 BsAb; 10 mM citrate buffer system; a pH of 6.9-7.1; 100-320 mM sugar component; and 0.9-1.1 mg/mL polysorbate 80.
M936. 20 mg/mL EGFR/LGR5 BsAb; 10 mM citrate buffer system; a pH of 6.9-7.1; 100-320 mM sugar component; and 0.9-1.1 mg/mL polysorbate 80.
M937. 1-150 mg/mL EGFR/LGR5 BsAb; 2-70 mM buffer system; a pH of 5.4-7.2; 150-350 mM sucrose; and 0.9-1.1 mg/mL polysorbate 80.
M938. 15-25 mg/mL EGFR/LGR5 BsAb; 2-70 mM buffer system; a pH of 5.4-7.2; 150-350 mM sucrose; and 0.9-1.1 mg/mL polysorbate 80.
M939. 20 mg/mL EGFR/LGR5 BsAb; 2-70 mM buffer system; a pH of 5.4-7.2; 150-350 mM sucrose; and 0.9-1.1 mg/mL polysorbate 80.
M940. 1-150 mg/mL EGFR/LGR5 BsAb; 3-12 mM histidine buffer system; a pH of 5.4-7.2; 150-350 mM sucrose; and 0.9-1.1 mg/mL polysorbate 80.
M941. 15-25 mg/mL EGFR/LGR5 BsAb; 3-12 mM histidine buffer system; a pH of 5.4-7.2; 150-350 mM sucrose; and 0.9-1.1 mg/mL polysorbate 80.
M942. 20 mg/mL EGFR/LGR5 BsAb; 3-12 mM histidine buffer system; a pH of 5.4-7.2; 150-350 mM sucrose; and 0.9-1.1 mg/mL polysorbate 80.
M943. 1-150 mg/mL EGFR/LGR5 BsAb; 5 mM histidine buffer system; a pH of 5.4-7.2; 150-350 mM sucrose; and 0.9-1.1 mg/mL polysorbate 80.
M944. 15-25 mg/mL EGFR/LGR5 BsAb; 5 mM histidine buffer system; a pH of 5.4-7.2; 150-350 mM sucrose; and 0.9-1.1 mg/mL polysorbate 80.
M945. 20 mg/mL EGFR/LGR5 BsAb; 5 mM histidine buffer system; a pH of 5.4-7.2; 150-350 mM sucrose; and 0.9-1.1 mg/mL polysorbate 80.
M946. 1-150 mg/mL EGFR/LGR5 BsAb; 10 mM histidine buffer system; a pH of 5.4-7.2; 150-350 mM sucrose; and 0.9-1.1 mg/mL polysorbate 80.
M947. 15-25 mg/mL EGFR/LGR5 BsAb; 10 mM histidine buffer system; a pH of 5.4-7.2; 150-350 mM sucrose; and 0.9-1.1 mg/mL polysorbate 80.
M948. 20 mg/mL EGFR/LGR5 BsAb; 10 mM histidine buffer system; a pH of 5.4-7.2; 150-350 mM sucrose; and 0.9-1.1 mg/mL polysorbate 80.
M949. 1-150 mg/mL EGFR/LGR5 BsAb; 3-12 mM citrate buffer system; a pH of 5.8-7.8; 150-350 mM sucrose; and 0.9-1.1 mg/mL polysorbate 80.
M950. 15-25 mg/mL EGFR/LGR5 BsAb; 3-12 mM citrate buffer system; a pH of 5.8-7.8; 150-350 mM sucrose; and 0.9-1.1 mg/mL polysorbate 80.
M951. 20 mg/mL EGFR/LGR5 BsAb; 3-12 mM citrate buffer system; a pH of 5.8-7.8; 150-350 mM sucrose; and 0.9-1.1 mg/mL polysorbate 80.
M952. 1-150 mg/mL EGFR/LGR5 BsAb; 10 mM citrate buffer system; a pH of 5.8-7.8; 150-350 mM sucrose; and 0.9-1.1 mg/mL polysorbate 80.
M953. 15-25 mg/mL EGFR/LGR5 BsAb; 10 mM citrate buffer system; a pH of 5.8-7.8; 150-350 mM sucrose; and 0.9-1.1 mg/mL polysorbate 80.
M954. 20 mg/mL EGFR/LGR5 BsAb; 10 mM citrate buffer system; a pH of 5.8-7.8; 150-350 mM sucrose; and 0.9-1.1 mg/mL polysorbate 80.
M955. 1-150 mg/mL EGFR/LGR5 BsAb; 2-70 mM buffer system; a pH of 5.8-6.0; 150-350 mM sucrose; and 0.9-1.1 mg/mL polysorbate 80.
M956. 15-25 mg/mL EGFR/LGR5 BsAb; 2-70 mM buffer system; a pH of 5.8-6.0; 150-350 mM sucrose; and 0.9-1.1 mg/mL polysorbate 80.
M957. 20 mg/mL EGFR/LGR5 BsAb; 2-70 mM buffer system; a pH of 5.8-6.0; 150-350 mM sucrose; and 0.9-1.1 mg/mL polysorbate 80.
M958. 1-150 mg/mL EGFR/LGR5 BsAb; 3-12 mM histidine buffer system; a pH of 5.8-6.0; 150-350 mM sucrose; and 0.9-1.1 mg/mL polysorbate 80.
M959. 15-25 mg/mL EGFR/LGR5 BsAb; 3-12 mM histidine buffer system; a pH of 5.8-6.0; 150-350 mM sucrose; and 0.9-1.1 mg/mL polysorbate 80.
M960. 20 mg/mL EGFR/LGR5 BsAb; 3-12 mM histidine buffer system; a pH of 5.8-6.0; 150-350 mM sucrose; and 0.9-1.1 mg/mL polysorbate 80.
M961. 1-150 mg/mL EGFR/LGR5 BsAb; 5 mM histidine buffer system; a pH of 5.8-6.0; 150-350 mM sucrose; and 0.9-1.1 mg/mL polysorbate 80.
M962. 15-25 mg/mL EGFR/LGR5 BsAb; 5 mM histidine buffer system; a pH of 5.8-6.0; 150-350 mM sucrose; and 0.9-1.1 mg/mL polysorbate 80.
M963. 20 mg/mL EGFR/LGR5 BsAb; 5 mM histidine buffer system; a pH of 5.8-6.0; 150-350 mM sucrose; and 0.9-1.1 mg/mL polysorbate 80.
M964. 1-150 mg/mL EGFR/LGR5 BsAb; 10 mM histidine buffer system; a pH of 5.8-6.0; 150-350 mM sucrose; and 0.9-1.1 mg/mL polysorbate 80.
M965. 15-25 mg/mL EGFR/LGR5 BsAb; 10 mM histidine buffer system; a pH of 5.8-6.0; 150-350 mM sucrose; and 0.9-1.1 mg/mL polysorbate 80.
M966. 20 mg/mL EGFR/LGR5 BsAb; 10 mM histidine buffer system; a pH of 5.8-6.0; 150-350 mM sucrose; and 0.9-1.1 mg/mL polysorbate 80.
M967. 1-150 mg/mL EGFR/LGR5 BsAb; 3-12 mM citrate buffer system; a pH of 5.8-7; 150-350 mM sucrose; and 0.9-1.1 mg/mL polysorbate 80.
M968. 15-25 mg/mL EGFR/LGR5 BsAb; 3-12 mM citrate buffer system; a pH of 5.8-7; 150-350 mM sucrose; and 0.9-1.1 mg/mL polysorbate 80.
M969. 20 mg/mL EGFR/LGR5 BsAb; 3-12 mM citrate buffer system; a pH of 5.8-7; 150-350 mM sucrose; and 0.9-1.1 mg/mL polysorbate 80.
M970. 1-150 mg/mL EGFR/LGR5 BsAb; 10 mM citrate buffer system; a pH of 5.8-7; 150-350 mM sucrose; and 0.9-1.1 mg/mL polysorbate 80.
M971. 15-25 mg/mL EGFR/LGR5 BsAb; 10 mM citrate buffer system; a pH of 5.8-7; 150-350 mM sucrose; and 0.9-1.1 mg/mL polysorbate 80.
M972. 20 mg/mL EGFR/LGR5 BsAb; 10 mM citrate buffer system; a pH of 5.8-7; 150-350 mM sucrose; and 0.9-1.1 mg/mL polysorbate 80.
M973. 1-150 mg/mL EGFR/LGR5 BsAb; 2-70 mM buffer system; a pH of 6.2-6.4; 150-350 mM sucrose; and 0.9-1.1 mg/mL polysorbate 80.
M974. 15-25 mg/mL EGFR/LGR5 BsAb; 2-70 mM buffer system; a pH of 6.2-6.4; 150-350 mM sucrose; and 0.9-1.1 mg/mL polysorbate 80.
M975. 20 mg/mL EGFR/LGR5 BsAb; 2-70 mM buffer system; a pH of 6.2-6.4; 150-350 mM sucrose; and 0.9-1.1 mg/mL polysorbate 80.
M976. 1-150 mg/mL EGFR/LGR5 BsAb; 3-12 mM histidine buffer system; a pH of 6.2-6.4; 150-350 mM sucrose; and 0.9-1.1 mg/mL polysorbate 80.
M977. 15-25 mg/mL EGFR/LGR5 BsAb; 3-12 mM histidine buffer system; a pH of 6.2-6.4; 150-350 mM sucrose; and 0.9-1.1 mg/mL polysorbate 80.
M978. 20 mg/mL EGFR/LGR5 BsAb; 3-12 mM histidine buffer system; a pH of 6.2-6.4; 150-350 mM sucrose; and 0.9-1.1 mg/mL polysorbate 80.
M979. 1-150 mg/mL EGFR/LGR5 BsAb; 5 mM histidine buffer system; a pH of 6.2-6.4; 150-350 mM sucrose; and 0.9-1.1 mg/mL polysorbate 80.
M980. 15-25 mg/mL EGFR/LGR5 BsAb; 5 mM histidine buffer system; a pH of 6.2-6.4; 150-350 mM sucrose; and 0.9-1.1 mg/mL polysorbate 80.
M981. 20 mg/mL EGFR/LGR5 BsAb; 5 mM histidine buffer system; a pH of 6.2-6.4; 150-350 mM sucrose; and 0.9-1.1 mg/mL polysorbate 80.
M982. 1-150 mg/mL EGFR/LGR5 BsAb; 10 mM histidine buffer system; a pH of 6.2-6.4; 150-350 mM sucrose; and 0.9-1.1 mg/mL polysorbate 80.
M983. 15-25 mg/mL EGFR/LGR5 BsAb; 10 mM histidine buffer system; a pH of 6.2-6.4; 150-350 mM sucrose; and 0.9-1.1 mg/mL polysorbate 80.
M984. 20 mg/mL EGFR/LGR5 BsAb; 10 mM histidine buffer system; a pH of 6.2-6.4; 150-350 mM sucrose; and 0.9-1.1 mg/mL polysorbate 80.
M985. 1-150 mg/mL EGFR/LGR5 BsAb; 3-12 mM citrate buffer system; a pH of 6.2-7.4; 150-350 mM sucrose; and 0.9-1.1 mg/mL polysorbate 80.
M986. 15-25 mg/mL EGFR/LGR5 BsAb; 3-12 mM citrate buffer system; a pH of 6.2-7.4; 150-350 mM sucrose; and 0.9-1.1 mg/mL polysorbate 80.
M987. 20 mg/mL EGFR/LGR5 BsAb; 3-12 mM citrate buffer system; a pH of 6.2-7.4; 150-350 mM sucrose; and 0.9-1.1 mg/mL polysorbate 80.
M988. 1-150 mg/mL EGFR/LGR5 BsAb; 10 mM citrate buffer system; a pH of 6.2-7.4; 150-350 mM sucrose; and 0.9-1.1 mg/mL polysorbate 80.
M989. 15-25 mg/mL EGFR/LGR5 BsAb; 10 mM citrate buffer system; a pH of 6.2-7.4; 150-350 mM sucrose; and 0.9-1.1 mg/mL polysorbate 80.
M990. 20 mg/mL EGFR/LGR5 BsAb; 10 mM citrate buffer system; a pH of 6.2-7.4; 150-350 mM sucrose; and 0.9-1.1 mg/mL polysorbate 80.
M991. 1-150 mg/mL EGFR/LGR5 BsAb; 2-70 mM buffer system; a pH of 6.9-7.1; 150-350 mM sucrose; and 0.9-1.1 mg/mL polysorbate 80.
M992. 15-25 mg/mL EGFR/LGR5 BsAb; 2-70 mM buffer system; a pH of 6.9-7.1; 150-350 mM sucrose; and 0.9-1.1 mg/mL polysorbate 80.
M993. 20 mg/mL EGFR/LGR5 BsAb; 2-70 mM buffer system; a pH of 6.9-7.1; 150-350 mM sucrose; and 0.9-1.1 mg/mL polysorbate 80.
M994. 1-150 mg/mL EGFR/LGR5 BsAb; 3-12 mM histidine buffer system; a pH of 6.9-7.1; 150-350 mM sucrose; and 0.9-1.1 mg/mL polysorbate 80.
M995. 15-25 mg/mL EGFR/LGR5 BsAb; 3-12 mM histidine buffer system; a pH of 6.9-7.1; 150-350 mM sucrose; and 0.9-1.1 mg/mL polysorbate 80.
M996. 20 mg/mL EGFR/LGR5 BsAb; 3-12 mM histidine buffer system; a pH of 6.9-7.1; 150-350 mM sucrose; and 0.9-1.1 mg/mL polysorbate 80.
M997. 1-150 mg/mL EGFR/LGR5 BsAb; 5 mM histidine buffer system; a pH of 6.9-7.1; 150-350 mM sucrose; and 0.9-1.1 mg/mL polysorbate 80.
M998. 15-25 mg/mL EGFR/LGR5 BsAb; 5 mM histidine buffer system; a pH of 6.9-7.1; 150-350 mM sucrose; and 0.9-1.1 mg/mL polysorbate 80.
M999. 20 mg/mL EGFR/LGR5 BsAb; 5 mM histidine buffer system; a pH of 6.9-7.1; 150-350 mM sucrose; and 0.9-1.1 mg/mL polysorbate 80.
M1000. 1-150 mg/mL EGFR/LGR5 BsAb; 10 mM histidine buffer system; a pH of 6.9-7.1; 150-350 mM sucrose; and 0.9-1.1 mg/mL polysorbate 80.
M1001. 15-25 mg/mL EGFR/LGR5 BsAb; 10 mM histidine buffer system; a pH of 6.9-7.1; 150-350 mM sucrose; and 0.9-1.1 mg/mL polysorbate 80.
M1002. 20 mg/mL EGFR/LGR5 BsAb; 10 mM histidine buffer system; a pH of 6.9-7.1; 150-350 mM sucrose; and 0.9-1.1 mg/mL polysorbate 80.
M1003. 1-150 mg/mL EGFR/LGR5 BsAb; 3-12 mM citrate buffer system; a pH of 6.9-7.1; 150-350 mM sucrose; and 0.9-1.1 mg/mL polysorbate 80.
M1004. 15-25 mg/mL EGFR/LGR5 BsAb; 3-12 mM citrate buffer system; a pH of 6.9-7.1; 150-350 mM sucrose; and 0.9-1.1 mg/mL polysorbate 80.
M1005. 20 mg/mL EGFR/LGR5 BsAb; 3-12 mM citrate buffer system; a pH of 6.9-7.1; 150-350 mM sucrose; and 0.9-1.1 mg/mL polysorbate 80.
M1006. 1-150 mg/mL EGFR/LGR5 BsAb; 10 mM citrate buffer system; a pH of 6.9-7.1; 150-350 mM sucrose; and 0.9-1.1 mg/mL polysorbate 80.
M1007. 15-25 mg/mL EGFR/LGR5 BsAb; 10 mM citrate buffer system; a pH of 6.9-7.1; 150-350 mM sucrose; and 0.9-1.1 mg/mL polysorbate 80.
M1008. 20 mg/mL EGFR/LGR5 BsAb; 10 mM citrate buffer system; a pH of 6.9-7.1; 150-350 mM sucrose; and 0.9-1.1 mg/mL polysorbate 80.
M1009. 1-150 mg/mL EGFR/LGR5 BsAb; 2-70 mM buffer system; a pH of 5.4-7.2; 275-295 mM sucrose; and 0.9-1.1 mg/mL polysorbate 80.
M1010. 15-25 mg/mL EGFR/LGR5 BsAb; 2-70 mM buffer system; a pH of 5.4-7.2; 275-295 mM sucrose; and 0.9-1.1 mg/mL polysorbate 80.
M1011. 20 mg/mL EGFR/LGR5 BsAb; 2-70 mM buffer system; a pH of 5.4-7.2; 275-295 mM sucrose; and 0.9-1.1 mg/mL polysorbate 80.
M1012. 1-150 mg/mL EGFR/LGR5 BsAb; 3-12 mM histidine buffer system; a pH of 5.4-7.2; 275-295 mM sucrose; and 0.9-1.1 mg/mL polysorbate 80.
M1013. 15-25 mg/mL EGFR/LGR5 BsAb; 3-12 mM histidine buffer system; a pH of 5.4-7.2; 275-295 mM sucrose; and 0.9-1.1 mg/mL polysorbate 80.
M1014. 20 mg/mL EGFR/LGR5 BsAb; 3-12 mM histidine buffer system; a pH of 5.4-7.2; 275-295 mM sucrose; and 0.9-1.1 mg/mL polysorbate 80.
M1015. 1-150 mg/mL EGFR/LGR5 BsAb; 5 mM histidine buffer system; a pH of 5.4-7.2; 275-295 mM sucrose; and 0.9-1.1 mg/mL polysorbate 80.
M1016. 15-25 mg/mL EGFR/LGR5 BsAb; 5 mM histidine buffer system; a pH of 5.4-7.2; 275-295 mM sucrose; and 0.9-1.1 mg/mL polysorbate 80.
M1017. 20 mg/mL EGFR/LGR5 BsAb; 5 mM histidine buffer system; a pH of 5.4-7.2; 275-295 mM sucrose; and 0.9-1.1 mg/mL polysorbate 80.
M1018. 1-150 mg/mL EGFR/LGR5 BsAb; 10 mM histidine buffer system; a pH of 5.4-7.2; 275-295 mM sucrose; and 0.9-1.1 mg/mL polysorbate 80.
M1019. 15-25 mg/mL EGFR/LGR5 BsAb; 10 mM histidine buffer system; a pH of 5.4-7.2; 275-295 mM sucrose; and 0.9-1.1 mg/mL polysorbate 80.
M1020. 20 mg/mL EGFR/LGR5 BsAb; 10 mM histidine buffer system; a pH of 5.4-7.2; 275-295 mM sucrose; and 0.9-1.1 mg/mL polysorbate 80.
M1021. 1-150 mg/mL EGFR/LGR5 BsAb; 3-12 mM citrate buffer system; a pH of 5.8-7.8; 275-295 mM sucrose; and 0.9-1.1 mg/mL polysorbate 80.
M1022. 15-25 mg/mL EGFR/LGR5 BsAb; 3-12 mM citrate buffer system; a pH of 5.8-7.8; 275-295 mM sucrose; and 0.9-1.1 mg/mL polysorbate 80.
M1023. 20 mg/mL EGFR/LGR5 BsAb; 3-12 mM citrate buffer system; a pH of 5.8-7.8; 275-295 mM sucrose; and 0.9-1.1 mg/mL polysorbate 80.
M1024. 1-150 mg/mL EGFR/LGR5 BsAb; 10 mM citrate buffer system; a pH of 5.8-7.8; 275-295 mM sucrose; and 0.9-1.1 mg/mL polysorbate 80.
M1025. 15-25 mg/mL EGFR/LGR5 BsAb; 10 mM citrate buffer system; a pH of 5.8-7.8; 275-295 mM sucrose; and 0.9-1.1 mg/mL polysorbate 80.
M1026. 20 mg/mL EGFR/LGR5 BsAb; 10 mM citrate buffer system; a pH of 5.8-7.8; 275-295 mM sucrose; and 0.9-1.1 mg/mL polysorbate 80.
M1027. 1-150 mg/mL EGFR/LGR5 BsAb; 2-70 mM buffer system; a pH of 5.8-6.0; 275-295 mM sucrose; and 0.9-1.1 mg/mL polysorbate 80.
M1028. 15-25 mg/mL EGFR/LGR5 BsAb; 2-70 mM buffer system; a pH of 5.8-6.0; 275-295 mM sucrose; and 0.9-1.1 mg/mL polysorbate 80.
M1029. 20 mg/mL EGFR/LGR5 BsAb; 2-70 mM buffer system; a pH of 5.8-6.0; 275-295 mM sucrose; and 0.9-1.1 mg/mL polysorbate 80.
M1030. 1-150 mg/mL EGFR/LGR5 BsAb; 3-12 mM histidine buffer system; a pH of 5.8-6.0; 275-295 mM sucrose; and 0.9-1.1 mg/mL polysorbate 80.
M1031. 15-25 mg/mL EGFR/LGR5 BsAb; 3-12 mM histidine buffer system; a pH of 5.8-6.0; 275-295 mM sucrose; and 0.9-1.1 mg/mL polysorbate 80.
M1032. 20 mg/mL EGFR/LGR5 BsAb; 3-12 mM histidine buffer system; a pH of 5.8-6.0; 275-295 mM sucrose; and 0.9-1.1 mg/mL polysorbate 80.
M1033. 1-150 mg/mL EGFR/LGR5 BsAb; 5 mM histidine buffer system; a pH of 5.8-6.0; 275-295 mM sucrose; and 0.9-1.1 mg/mL polysorbate 80.
M1034. 15-25 mg/mL EGFR/LGR5 BsAb; 5 mM histidine buffer system; a pH of 5.8-6.0; 275-295 mM sucrose; and 0.9-1.1 mg/mL polysorbate 80.
M1035. 20 mg/mL EGFR/LGR5 BsAb; 5 mM histidine buffer system; a pH of 5.8-6.0; 275-295 mM sucrose; and 0.9-1.1 mg/mL polysorbate 80.
M1036. 1-150 mg/mL EGFR/LGR5 BsAb; 10 mM histidine buffer system; a pH of 5.8-6.0; 275-295 mM sucrose; and 0.9-1.1 mg/mL polysorbate 80.
M1037. 15-25 mg/mL EGFR/LGR5 BsAb; 10 mM histidine buffer system; a pH of 5.8-6.0; 275-295 mM sucrose; and 0.9-1.1 mg/mL polysorbate 80.
M1038. 20 mg/mL EGFR/LGR5 BsAb; 10 mM histidine buffer system; a pH of 5.8-6.0; 275-295 mM sucrose; and 0.9-1.1 mg/mL polysorbate 80.
M1039. 1-150 mg/mL EGFR/LGR5 BsAb; 3-12 mM citrate buffer system; a pH of 5.8-7; 275-295 mM sucrose; and 0.9-1.1 mg/mL polysorbate 80.
M1040. 15-25 mg/mL EGFR/LGR5 BsAb; 3-12 mM citrate buffer system; a pH of 5.8-7; 275-295 mM sucrose; and 0.9-1.1 mg/mL polysorbate 80.
M1041. 20 mg/mL EGFR/LGR5 BsAb; 3-12 mM citrate buffer system; a pH of 5.8-7; 275-295 mM sucrose; and 0.9-1.1 mg/mL polysorbate 80.
M1042. 1-150 mg/mL EGFR/LGR5 BsAb; 10 mM citrate buffer system; a pH of 5.8-7; 275-295 mM sucrose; and 0.9-1.1 mg/mL polysorbate 80.
M1043. 15-25 mg/mL EGFR/LGR5 BsAb; 10 mM citrate buffer system; a pH of 5.8-7; 275-295 mM sucrose; and 0.9-1.1 mg/mL polysorbate 80.
M1044. 20 mg/mL EGFR/LGR5 BsAb; 10 mM citrate buffer system; a pH of 5.8-7; 275-295 mM sucrose; and 0.9-1.1 mg/mL polysorbate 80.
M1045. 1-150 mg/mL EGFR/LGR5 BsAb; 2-70 mM buffer system; a pH of 6.2-6.4; 275-295 mM sucrose; and 0.9-1.1 mg/mL polysorbate 80.
M1046. 15-25 mg/mL EGFR/LGR5 BsAb; 2-70 mM buffer system; a pH of 6.2-6.4; 275-295 mM sucrose; and 0.9-1.1 mg/mL polysorbate 80.
M1047. 20 mg/mL EGFR/LGR5 BsAb; 2-70 mM buffer system; a pH of 6.2-6.4; 275-295 mM sucrose; and 0.9-1.1 mg/mL polysorbate 80.
M1048. 1-150 mg/mL EGFR/LGR5 BsAb; 3-12 mM histidine buffer system; a pH of 6.2-6.4; 275-295 mM sucrose; and 0.9-1.1 mg/mL polysorbate 80.
M1049. 15-25 mg/mL EGFR/LGR5 BsAb; 3-12 mM histidine buffer system; a pH of 6.2-6.4; 275-295 mM sucrose; and 0.9-1.1 mg/mL polysorbate 80.
M1050. 20 mg/mL EGFR/LGR5 BsAb; 3-12 mM histidine buffer system; a pH of 6.2-6.4; 275-295 mM sucrose; and 0.9-1.1 mg/mL polysorbate 80.
M1051. 1-150 mg/mL EGFR/LGR5 BsAb; 5 mM histidine buffer system; a pH of 6.2-6.4; 275-295 mM sucrose; and 0.9-1.1 mg/mL polysorbate 80.
M1052. 15-25 mg/mL EGFR/LGR5 BsAb; 5 mM histidine buffer system; a pH of 6.2-6.4; 275-295 mM sucrose; and 0.9-1.1 mg/mL polysorbate 80.
M1053. 20 mg/mL EGFR/LGR5 BsAb; 5 mM histidine buffer system; a pH of 6.2-6.4; 275-295 mM sucrose; and 0.9-1.1 mg/mL polysorbate 80.
M1054. 1-150 mg/mL EGFR/LGR5 BsAb; 10 mM histidine buffer system; a pH of 6.2-6.4; 275-295 mM sucrose; and 0.9-1.1 mg/mL polysorbate 80.
M1055. 15-25 mg/mL EGFR/LGR5 BsAb; 10 mM histidine buffer system; a pH of 6.2-6.4; 275-295 mM sucrose; and 0.9-1.1 mg/mL polysorbate 80.
M1056. 20 mg/mL EGFR/LGR5 BsAb; 10 mM histidine buffer system; a pH of 6.2-6.4; 275-295 mM sucrose; and 0.9-1.1 mg/mL polysorbate 80.
M1057. 1-150 mg/mL EGFR/LGR5 BsAb; 3-12 mM citrate buffer system; a pH of 6.2-7.4; 275-295 mM sucrose; and 0.9-1.1 mg/mL polysorbate 80.
M1058. 15-25 mg/mL EGFR/LGR5 BsAb; 3-12 mM citrate buffer system; a pH of 6.2-7.4; 275-295 mM sucrose; and 0.9-1.1 mg/mL polysorbate 80.
M1059. 20 mg/mL EGFR/LGR5 BsAb; 3-12 mM citrate buffer system; a pH of 6.2-7.4; 275-295 mM sucrose; and 0.9-1.1 mg/mL polysorbate 80.
M1060. 1-150 mg/mL EGFR/LGR5 BsAb; 10 mM citrate buffer system; a pH of 6.2-7.4; 275-295 mM sucrose; and 0.9-1.1 mg/mL polysorbate 80.
M1061. 15-25 mg/mL EGFR/LGR5 BsAb; 10 mM citrate buffer system; a pH of 6.2-7.4; 275-295 mM sucrose; and 0.9-1.1 mg/mL polysorbate 80.
M1062. 20 mg/mL EGFR/LGR5 BsAb; 10 mM citrate buffer system; a pH of 6.2-7.4; 275-295 mM sucrose; and 0.9-1.1 mg/mL polysorbate 80.
M1063. 1-150 mg/mL EGFR/LGR5 BsAb; 2-70 mM buffer system; a pH of 6.9-7.1; 275-295 mM sucrose; and 0.9-1.1 mg/mL polysorbate 80.
M1064. 15-25 mg/mL EGFR/LGR5 BsAb; 2-70 mM buffer system; a pH of 6.9-7.1; 275-295 mM sucrose; and 0.9-1.1 mg/mL polysorbate 80.
M1065. 20 mg/mL EGFR/LGR5 BsAb; 2-70 mM buffer system; a pH of 6.9-7.1; 275-295 mM sucrose; and 0.9-1.1 mg/mL polysorbate 80.
M1066. 1-150 mg/mL EGFR/LGR5 BsAb; 3-12 mM histidine buffer system; a pH of 6.9-7.1; 275-295 mM sucrose; and 0.9-1.1 mg/mL polysorbate 80.
M1067. 15-25 mg/mL EGFR/LGR5 BsAb; 3-12 mM histidine buffer system; a pH of 6.9-7.1; 275-295 mM sucrose; and 0.9-1.1 mg/mL polysorbate 80.
M1068. 20 mg/mL EGFR/LGR5 BsAb; 3-12 mM histidine buffer system; a pH of 6.9-7.1; 275-295 mM sucrose; and 0.9-1.1 mg/mL polysorbate 80.
M1069. 1-150 mg/mL EGFR/LGR5 BsAb; 5 mM histidine buffer system; a pH of 6.9-7.1; 275-295 mM sucrose; and 0.9-1.1 mg/mL polysorbate 80.
M1070. 15-25 mg/mL EGFR/LGR5 BsAb; 5 mM histidine buffer system; a pH of 6.9-7.1; 275-295 mM sucrose; and 0.9-1.1 mg/mL polysorbate 80.
M1071. 20 mg/mL EGFR/LGR5 BsAb; 5 mM histidine buffer system; a pH of 6.9-7.1; 275-295 mM sucrose; and 0.9-1.1 mg/mL polysorbate 80.
M1072. 1-150 mg/mL EGFR/LGR5 BsAb; 10 mM histidine buffer system; a pH of 6.9-7.1; 275-295 mM sucrose; and 0.9-1.1 mg/mL polysorbate 80.
M1073. 15-25 mg/mL EGFR/LGR5 BsAb; 10 mM histidine buffer system; a pH of 6.9-7.1; 275-295 mM sucrose; and 0.9-1.1 mg/mL polysorbate 80.
M1074. 20 mg/mL EGFR/LGR5 BsAb; 10 mM histidine buffer system; a pH of 6.9-7.1; 275-295 mM sucrose; and 0.9-1.1 mg/mL polysorbate 80.
M1075. 1-150 mg/mL EGFR/LGR5 BsAb; 3-12 mM citrate buffer system; a pH of 6.9-7.1; 275-295 mM sucrose; and 0.9-1.1 mg/mL polysorbate 80.
M1076. 15-25 mg/mL EGFR/LGR5 BsAb; 3-12 mM citrate buffer system; a pH of 6.9-7.1; 275-295 mM sucrose; and 0.9-1.1 mg/mL polysorbate 80.
M1077. 20 mg/mL EGFR/LGR5 BsAb; 3-12 mM citrate buffer system; a pH of 6.9-7.1; 275-295 mM sucrose; and 0.9-1.1 mg/mL polysorbate 80.
M1078. 1-150 mg/mL EGFR/LGR5 BsAb; 10 mM citrate buffer system; a pH of 6.9-7.1; 275-295 mM sucrose; and 0.9-1.1 mg/mL polysorbate 80.
M1079. 15-25 mg/mL EGFR/LGR5 BsAb; 10 mM citrate buffer system; a pH of 6.9-7.1; 275-295 mM sucrose; and 0.9-1.1 mg/mL polysorbate 80.
M1080. 20 mg/mL EGFR/LGR5 BsAb; 10 mM citrate buffer system; a pH of 6.9-7.1; 275-295 mM sucrose; and 0.9-1.1 mg/mL polysorbate 80.

In a particular embodiment, the pharmaceutical composition is an aqueous composition consisting of 15-30 mg/mL EGFR/LGR5 BsAb, 3-12 mM histidine buffer system, 250-300 mM sucrose, 0.8-1.2 mg/mL polysorbate 80 with a pH of 6.1-6.5.

In a particular embodiment, the pharmaceutical composition is an aqueous composition consisting of 20 mg/mL EGFR/LGR5, 5 mM histidine buffer system, 290 mM sucrose, 1 mg/mL polysorbate 80 with of 6.3.

In a particular embodiment, the pharmaceutical composition is an aqueous composition consisting of 15-30 mg/mL of a bispecific antibody comprising a first binding domain binding to EGFR and a second binding domain binding to LGR5, 3-12 mM histidine buffer system, 250-300 mM sucrose, 0.3-0.7 mg/mL polysorbate 80 with a pH of of 5.7-6.1.

In a particular embodiment, the pharmaceutical composition is an aqueous composition consisting of 20 mg/mL of a bispecific antibody comprising a first binding domain binding to EGFR and a second binding domain binding to LGR5, 10 mM histidine buffer system, 280 mM sucrose, 0.5 mg/mL polysorbate 80 with a pH of 5.9.

In a particular embodiment, the pharmaceutical composition is an aqueous composition consisting of 15-30 mg/mL of a bispecific antibody comprising a first binding domain binding to EGFR and a second binding domain binding to LGR5, 3-12 mM histidine buffer system, 250-300 mM sucrose, 0.8-1.2 mg/mL polysorbate 80 with a pH of 5.7-6.1.

In a particular embodiment, the pharmaceutical composition is an aqueous composition consisting of 20 mg/mL of a bispecific antibody comprising a first binding domain binding to EGFR and a second binding domain binding to LGR5, 10 mM histidine buffer system, 280 mM sucrose, 1 mg/mL polysorbate 80 with a pH of 5.9.

In a particular embodiment, the pharmaceutical composition is an aqueous composition consisting of 15-30 mg/mL of a bispecific antibody comprising a first binding domain binding to EGFR and a second binding domain binding to LGR5, 3-12 mM citrate buffer system (preferable sodium citrate buffer system), 250-300 mM sucrose, 0.3-0.7 mg/mL polysorbate 80 with a pH of 6.8-7.2.

In a particular embodiment, the pharmaceutical composition is an aqueous composition consisting of 20 mg/mL of a bispecific antibody comprising a first binding domain binding to EGFR and a second binding domain binding to LGR5, 10 mM citrate buffer system (preferable sodium citrate buffer system), 280 mM sucrose, 0.5 mg/mL polysorbate 80 with a pH of 7.

Several other plausibly viable formulations arise from the studies undertaken to develop the present invention. As such:
N) the following numbered paragraphs N1 to N2686 disclose specific embodiments of the invention, in which the pharmaceutical composition comprises (or consists of, optionally along with water for injection in the case of aqueous compositions) and is characterised by:
N1. 1-150 mg/mL EGFR/LGR5 BsAb; and 3-25 mM histidine buffer system providing a composition pH of 5.4-7.1.
N2. 10-60 mg/mL EGFR/LGR5 BsAb; and 3-25 mM histidine buffer system providing a composition pH of 5.4-7.1.
N3. 1-150 mg/mL EGFR/LGR5 BsAb; and 3-25 mM acetate buffer system providing a composition pH of 4-5.6.
N4. 10-60 mg/mL EGFR/LGR5 BsAb; and 3-25 mM acetate buffer system providing a composition pH of 4-5.6.
N5. 1-150 mg/mL EGFR/LGR5 BsAb; and 3-25 mM citrate buffer system providing a composition pH of 4.5-7.5.
N6. 10-60 mg/mL EGFR/LGR5 BsAb; and 3-25 mM citrate buffer system providing a composition pH of 4.5-7.5.
N7. 1-150 mg/mL EGFR/LGR5 BsAb; and 3-25 mM succinate buffer system providing a composition pH of 4-6.6.
N8. 10-60 mg/mL EGFR/LGR5 BsAb; and 3-25 mM succinate buffer system providing a composition pH of 4-6.6.
N9. 1-150 mg/mL EGFR/LGR5 BsAb; and 3-25 mM phosphate buffer system providing a composition pH of 5.8-8.1.
N10. 10-60 mg/mL EGFR/LGR5 BsAb; and 3-25 mM phosphate buffer system providing a composition pH of 5.8-8.1.
N11. 1-150 mg/mL EGFR/LGR5 BsAb; and 3-25 mM citrate-phosphate buffer system providing a composition pH of 4-7.1.
N12. 10-60 mg/mL EGFR/LGR5 BsAb; and 3-25 mM citrate-phosphate buffer system providing a composition pH of 4-7.1.
N13. 1-150 mg/mL EGFR/LGR5 BsAb; and 10-180 mM sodium chloride.
N14. 10-60 mg/mL EGFR/LGR5 BsAb; and 10-180 mM sodium chloride.
N15. 1-150 mg/mL EGFR/LGR5 BsAb; and 100-400 mM sugar component selected from the group consisting of sucrose, mannitol, sorbitol, and maltose.
N16. 10-60 mg/mL EGFR/LGR5 BsAb; and 100-400 mM sugar component selected from the group consisting of sucrose, mannitol, sorbitol, and maltose.
N17. 1-150 mg/mL EGFR/LGR5 BsAb; and 150-350 mM sucrose.
N18. 10-60 mg/mL EGFR/LGR5 BsAb; and 150-350 mM sucrose.
N19. 1-150 mg/mL EGFR/LGR5 BsAb; and 150-350 mM maltose.
N20. 10-60 mg/mL EGFR/LGR5 BsAb; and 150-350 mM maltose.
N21. 1-150 mg/mL EGFR/LGR5 BsAb; and 0.4-1.2 mg/mL surfactant.
N22. 10-60 mg/mL EGFR/LGR5 BsAb; and 0.4-1.2 mg/mL surfactant.
N23. 1-150 mg/mL EGFR/LGR5 BsAb; and 0.1-1.1 mg/mL polysorbate 80.
N24. 10-60 mg/mL EGFR/LGR5 BsAb; and 0.1-1.1 mg/mL polysorbate 80.
N25. 1-150 mg/mL EGFR/LGR5 BsAb; and 5-150 mM amino acid component selected from the group consisting of proline, glycine, lysine, and any combination thereof.
N26. 10-60 mg/mL EGFR/LGR5 BsAb; and 5-150 mM amino acid component selected from the group consisting of proline, glycine, lysine, and any combination thereof.
N27. 1-150 mg/mL EGFR/LGR5 BsAb; and 2-25 mM methionine.
N28. 10-60 mg/mL EGFR/LGR5 BsAb; and 2-25 mM methionine.
N29. 1-150 mg/mL EGFR/LGR5 BsAb; and 0.01-0.2 mM EDTA.
N30. 10-60 mg/mL EGFR/LGR5 BsAb; and 0.01-0.2 mM EDTA.
N31. 1-150 mg/mL EGFR/LGR5 BsAb; 3-25 mM histidine buffer system providing a composition pH of 5.4-7.1; and 10-180 mM sodium chloride.
N32. 10-60 mg/mL EGFR/LGR5 BsAb; 3-25 mM histidine buffer system providing a composition pH of 5.4-7.1; and 10-180 mM sodium chloride.
N33. 1-150 mg/mL EGFR/LGR5 BsAb; 3-25 mM acetate buffer system providing a composition pH of 4-5.6; and 10-180 mM sodium chloride.
N34. 10-60 mg/mL EGFR/LGR5 BsAb; 3-25 mM acetate buffer system providing a composition pH of 4-5.6; and 10-180 mM sodium chloride.
N35. 1-150 mg/mL EGFR/LGR5 BsAb; 3-25 mM citrate buffer system providing a composition pH of 4.5-7.5; and 10-180 mM sodium chloride.
N36. 10-60 mg/mL EGFR/LGR5 BsAb; 3-25 mM citrate buffer system providing a composition pH of 4.5-7.5; and 10-180 mM sodium chloride.
N37. 1-150 mg/mL EGFR/LGR5 BsAb; 3-25 mM succinate buffer system providing a composition pH of 4-6.6; and 10-180 mM sodium chloride.
N38. 10-60 mg/mL EGFR/LGR5 BsAb; 3-25 mM succinate buffer system providing a composition pH of 4-6.6; and 10-180 mM sodium chloride.
N39. 1-150 mg/mL EGFR/LGR5 BsAb; 3-25 mM phosphate buffer system providing a composition pH of 5.8-8.1; and 10-180 mM sodium chloride.
N40. 10-60 mg/mL EGFR/LGR5 BsAb; 3-25 mM phosphate buffer system providing a composition pH of 5.8-8.1; and 10-180 mM sodium chloride.
N41. 1-150 mg/mL EGFR/LGR5 BsAb; 3-25 mM citrate-phosphate buffer system providing a composition pH of 4-7.1; and 10-180 mM sodium chloride.
N42. 10-60 mg/mL EGFR/LGR5 BsAb; 3-25 mM citrate-phosphate buffer system providing a composition pH of 4-7.1; and 10-180 mM sodium chloride.
N43. 1-150 mg/mL EGFR/LGR5 BsAb; 3-25 mM histidine buffer system providing a composition pH of 5.4-7.1; and 100-400 mM sugar component selected from the group consisting of sucrose, mannitol, sorbitol, and maltose.
N44. 10-60 mg/mL EGFR/LGR5 BsAb; 3-25 mM histidine buffer system providing a composition pH of 5.4-7.1; and 100-400 mM sugar component selected from the group consisting of sucrose, mannitol, sorbitol, and maltose.
N45. 1-150 mg/mL EGFR/LGR5 BsAb; 3-25 mM acetate buffer system providing a composition pH of 4-5.6; and 100-400 mM sugar component selected from the group consisting of sucrose, mannitol, sorbitol, and maltose.
N46. 10-60 mg/mL EGFR/LGR5 BsAb; 3-25 mM acetate buffer system providing a composition pH of 4-5.6; and 100-400 mM sugar component selected from the group consisting of sucrose, mannitol, sorbitol, and maltose.
N47. 1-150 mg/mL EGFR/LGR5 BsAb; 3-25 mM citrate buffer system providing a composition pH of 4.5-7.5; and 100-400 mM sugar component selected from the group consisting of sucrose, mannitol, sorbitol, and maltose.
N48. 10-60 mg/mL EGFR/LGR5 BsAb; 3-25 mM citrate buffer system providing a composition pH of 4.5-7.5; and 100-400 mM sugar component selected from the group consisting of sucrose, mannitol, sorbitol, and maltose.
N49. 1-150 mg/mL EGFR/LGR5 BsAb; 3-25 mM succinate buffer system providing a composition pH of 4-6.6; and 100-400 mM sugar component selected from the group consisting of sucrose, mannitol, sorbitol, and maltose.
N50. 10-60 mg/mL EGFR/LGR5 BsAb; 3-25 mM succinate buffer system providing a composition pH of 4-6.6; and 100-400 mM sugar component selected from the group consisting of sucrose, mannitol, sorbitol, and maltose.
N51. 1-150 mg/mL EGFR/LGR5 BsAb; 3-25 mM phosphate buffer system providing a composition pH of 5.8-8.1; and 100-400 mM sugar component selected from the group consisting of sucrose, mannitol, sorbitol, and maltose.
N52. 10-60 mg/mL EGFR/LGR5 BsAb; 3-25 mM phosphate buffer system providing a composition pH of 5.8-8.1; and 100-400 mM sugar component selected from the group consisting of sucrose, mannitol, sorbitol, and maltose.
N53. 1-150 mg/mL EGFR/LGR5 BsAb; 3-25 mM citrate-phosphate buffer system providing a composition pH of 4-7.1; and 100-400 mM sugar component selected from the group consisting of sucrose, mannitol, sorbitol, and maltose.
N54. 10-60 mg/mL EGFR/LGR5 BsAb; 3-25 mM citrate-phosphate buffer system providing a composition pH of 4-7.1; and 100-400 mM sugar component selected from the group consisting of sucrose, mannitol, sorbitol, and maltose.
N55. 1-150 mg/mL EGFR/LGR5 BsAb; 3-25 mM histidine buffer system providing a composition pH of 5.4-7.1; and 150-350 mM sucrose.
N56. 10-60 mg/mL EGFR/LGR5 BsAb; 3-25 mM histidine buffer system providing a composition pH of 5.4-7.1; and 150-350 mM sucrose.
N57. 1-150 mg/mL EGFR/LGR5 BsAb; 3-25 mM acetate buffer system providing a composition pH of 4-5.6; and 150-350 mM sucrose.
N58. 10-60 mg/mL EGFR/LGR5 BsAb; 3-25 mM acetate buffer system providing a composition pH of 4-5.6; and 150-350 mM sucrose.
N59. 1-150 mg/mL EGFR/LGR5 BsAb; 3-25 mM citrate buffer system providing a composition pH of 4.5-7.5; and 150-350 mM sucrose.
N60. 10-60 mg/mL EGFR/LGR5 BsAb; 3-25 mM citrate buffer system providing a composition pH of 4.5-7.5; and 150-350 mM sucrose.
N61. 1-150 mg/mL EGFR/LGR5 BsAb; 3-25 mM succinate buffer system providing a composition pH of 4-6.6; and 150-350 mM sucrose.
N62. 10-60 mg/mL EGFR/LGR5 BsAb; 3-25 mM succinate buffer system providing a composition pH of 4-6.6; and 150-350 mM sucrose.
N63. 1-150 mg/mL EGFR/LGR5 BsAb; 3-25 mM phosphate buffer system providing a composition pH of 5.8-8.1; and 150-350 mM sucrose.
N64. 10-60 mg/mL EGFR/LGR5 BsAb; 3-25 mM phosphate buffer system providing a composition pH of 5.8-8.1; and 150-350 mM sucrose.
N65. 1-150 mg/mL EGFR/LGR5 BsAb; 3-25 mM citrate-phosphate buffer system providing a composition pH of 4-7.1; and 150-350 mM sucrose.
N66. 10-60 mg/mL EGFR/LGR5 BsAb; 3-25 mM citrate-phosphate buffer system providing a composition pH of 4-7.1; and 150-350 mM sucrose.
N67. 1-150 mg/mL EGFR/LGR5 BsAb; 3-25 mM histidine buffer system providing a composition pH of 5.4-7.1; and 150-350 mM maltose.
N68. 10-60 mg/mL EGFR/LGR5 BsAb; 3-25 mM histidine buffer system providing a composition pH of 5.4-7.1; and 150-350 mM maltose.
N69. 1-150 mg/mL EGFR/LGR5 BsAb; 3-25 mM acetate buffer system providing a composition pH of 4-5.6; and 150-350 mM maltose.
N70. 10-60 mg/mL EGFR/LGR5 BsAb; 3-25 mM acetate buffer system providing a composition pH of 4-5.6; and 150-350 mM maltose.
N71. 1-150 mg/mL EGFR/LGR5 BsAb; 3-25 mM citrate buffer system providing a composition pH of 4.5-7.5; and 150-350 mM maltose.
N72. 10-60 mg/mL EGFR/LGR5 BsAb; 3-25 mM citrate buffer system providing a composition pH of 4.5-7.5; and 150-350 mM maltose.
N73. 1-150 mg/mL EGFR/LGR5 BsAb; 3-25 mM succinate buffer system providing a composition pH of 4-6.6; and 150-350 mM maltose.
N74. 10-60 mg/mL EGFR/LGR5 BsAb; 3-25 mM succinate buffer system providing a composition pH of 4-6.6; and 150-350 mM maltose.
N75. 1-150 mg/mL EGFR/LGR5 BsAb; 3-25 mM phosphate buffer system providing a composition pH of 5.8-8.1; and 150-350 mM maltose.
N76. 10-60 mg/mL EGFR/LGR5 BsAb; 3-25 mM phosphate buffer system providing a composition pH of 5.8-8.1; and 150-350 mM maltose.
N77. 1-150 mg/mL EGFR/LGR5 BsAb; 3-25 mM citrate-phosphate buffer system providing a composition pH of 4-7.1; and 150-350 mM maltose.
N78. 10-60 mg/mL EGFR/LGR5 BsAb; 3-25 mM citrate-phosphate buffer system providing a composition pH of 4-7.1; and 150-350 mM maltose.
N79. 1-150 mg/mL EGFR/LGR5 BsAb; 3-25 mM histidine buffer system providing a composition pH of 5.4-7.1; and 0.4-1.2 mg/mL surfactant.
N80. 10-60 mg/mL EGFR/LGR5 BsAb; 3-25 mM histidine buffer system providing a composition pH of 5.4-7.1; and 0.4-1.2 mg/mL surfactant.
N81. 1-150 mg/mL EGFR/LGR5 BsAb; 3-25 mM acetate buffer system providing a composition pH of 4-5.6; and 0.4-1.2 mg/mL surfactant.
N82. 10-60 mg/mL EGFR/LGR5 BsAb; 3-25 mM acetate buffer system providing a composition pH of 4-5.6; and 0.4-1.2 mg/mL surfactant.
N83. 1-150 mg/mL EGFR/LGR5 BsAb; 3-25 mM citrate buffer system providing a composition pH of 4.5-7.5; and 0.4-1.2 mg/mL surfactant.
N84. 10-60 mg/mL EGFR/LGR5 BsAb; 3-25 mM citrate buffer system providing a composition pH of 4.5-7.5; and 0.4-1.2 mg/mL surfactant.
N85. 1-150 mg/mL EGFR/LGR5 BsAb; 3-25 mM succinate buffer system providing a composition pH of 4-6.6; and 0.4-1.2 mg/mL surfactant.
N86. 10-60 mg/mL EGFR/LGR5 BsAb; 3-25 mM succinate buffer system providing a composition pH of 4-6.6; and 0.4-1.2 mg/mL surfactant.
N87. 1-150 mg/mL EGFR/LGR5 BsAb; 3-25 mM phosphate buffer system providing a composition pH of 5.8-8.1; and 0.4-1.2 mg/mL surfactant.
N88. 10-60 mg/mL EGFR/LGR5 BsAb; 3-25 mM phosphate buffer system providing a composition pH of 5.8-8.1; and 0.4-1.2 mg/mL surfactant.
N89. 1-150 mg/mL EGFR/LGR5 BsAb; 3-25 mM citrate-phosphate buffer system providing a composition pH of 4-7.1; and 0.4-1.2 mg/mL surfactant.
N90. 10-60 mg/mL EGFR/LGR5 BsAb; 3-25 mM citrate-phosphate buffer system providing a composition pH of 4-7.1; and 0.4-1.2 mg/mL surfactant.
N91. 1-150 mg/mL EGFR/LGR5 BsAb; 3-25 mM histidine buffer system providing a composition pH of 5.4-7.1; and 0.1-1.1 mg/mL polysorbate 80.
N92. 10-60 mg/mL EGFR/LGR5 BsAb; 3-25 mM histidine buffer system providing a composition pH of 5.4-7.1; and 0.1-1.1 mg/mL polysorbate 80.
N93. 1-150 mg/mL EGFR/LGR5 BsAb; 3-25 mM acetate buffer system providing a composition pH of 4-5.6; and 0.1-1.1 mg/mL polysorbate 80.
N94. 10-60 mg/mL EGFR/LGR5 BsAb; 3-25 mM acetate buffer system providing a composition pH of 4-5.6; and 0.1-1.1 mg/mL polysorbate 80.
N95. 1-150 mg/mL EGFR/LGR5 BsAb; 3-25 mM citrate buffer system providing a composition pH of 4.5-7.5; and 0.1-1.1 mg/mL polysorbate 80.
N96. 10-60 mg/mL EGFR/LGR5 BsAb; 3-25 mM citrate buffer system providing a composition pH of 4.5-7.5; and 0.1-1.1 mg/mL polysorbate 80.
N97. 1-150 mg/mL EGFR/LGR5 BsAb; 3-25 mM succinate buffer system providing a composition pH of 4-6.6; and 0.1-1.1 mg/mL polysorbate 80.
N98. 10-60 mg/mL EGFR/LGR5 BsAb; 3-25 mM succinate buffer system providing a composition pH of 4-6.6; and 0.1-1.1 mg/mL polysorbate 80.
N99. 1-150 mg/mL EGFR/LGR5 BsAb; 3-25 mM phosphate buffer system providing a composition pH of 5.8-8.1; and 0.1-1.1 mg/mL polysorbate 80.
N100. 10-60 mg/mL EGFR/LGR5 BsAb; 3-25 mM phosphate buffer system providing a composition pH of 5.8-8.1; and 0.1-1.1 mg/mL polysorbate 80.
N101. 1-150 mg/mL EGFR/LGR5 BsAb; 3-25 mM citrate-phosphate buffer system providing a composition pH of 4-7.1; and 0.1-1.1 mg/mL polysorbate 80.
N102. 10-60 mg/mL EGFR/LGR5 BsAb; 3-25 mM citrate-phosphate buffer system providing a composition pH of 4-7.1; and 0.1-1.1 mg/mL polysorbate 80.
N103. 1-150 mg/mL EGFR/LGR5 BsAb; 3-25 mM histidine buffer system providing a composition pH of 5.4-7.1; and 5-150 mM amino acid component selected from the group consisting of proline, glycine, lysine, and any combination thereof.
N104. 10-60 mg/mL EGFR/LGR5 BsAb; 3-25 mM histidine buffer system providing a composition pH of 5.4-7.1; and 5-150 mM amino acid component selected from the group consisting of proline, glycine, lysine, and any combination thereof.
N105. 1-150 mg/mL EGFR/LGR5 BsAb; 3-25 mM acetate buffer system providing a composition pH of 4-5.6; and 5-150 mM amino acid component selected from the group consisting of proline, glycine, lysine, and any combination thereof.
N106. 10-60 mg/mL EGFR/LGR5 BsAb; 3-25 mM acetate buffer system providing a composition pH of 4-5.6; and 5-150 mM amino acid component selected from the group consisting of proline, glycine, lysine, and any combination thereof.
N107. 1-150 mg/mL EGFR/LGR5 BsAb; 3-25 mM citrate buffer system providing a composition pH of 4.5-7.5; and 5-150 mM amino acid component selected from the group consisting of proline, glycine, lysine, and any combination thereof.
N108. 10-60 mg/mL EGFR/LGR5 BsAb; 3-25 mM citrate buffer system providing a composition pH of 4.5-7.5; and 5-150 mM amino acid component selected from the group consisting of proline, glycine, lysine, and any combination thereof.
N109. 1-150 mg/mL EGFR/LGR5 BsAb; 3-25 mM succinate buffer system providing a composition pH of 4-6.6; and 5-150 mM amino acid component selected from the group consisting of proline, glycine, lysine, and any combination thereof.
N110. 10-60 mg/mL EGFR/LGR5 BsAb; 3-25 mM succinate buffer system providing a composition pH of 4-6.6; and 5-150 mM amino acid component selected from the group consisting of proline, glycine, lysine, and any combination thereof.
N111. 1-150 mg/mL EGFR/LGR5 BsAb; 3-25 mM phosphate buffer system providing a composition pH of 5.8-8.1; and 5-150 mM amino acid component selected from the group consisting of proline, glycine, lysine, and any combination thereof.
N112. 10-60 mg/mL EGFR/LGR5 BsAb; 3-25 mM phosphate buffer system providing a composition pH of 5.8-8.1; and 5-150 mM amino acid component selected from the group consisting of proline, glycine, lysine, and any combination thereof.
N113. 1-150 mg/mL EGFR/LGR5 BsAb; 3-25 mM citrate-phosphate buffer system providing a composition pH of 4-7.1; and 5-150 mM amino acid component selected from the group consisting of proline, glycine, lysine, and any combination thereof.
N114. 10-60 mg/mL EGFR/LGR5 BsAb; 3-25 mM citrate-phosphate buffer system providing a composition pH of 4-7.1; and 5-150 mM amino acid component selected from the group consisting of proline, glycine, lysine, and any combination thereof.
N115. 1-150 mg/mL EGFR/LGR5 BsAb; 3-25 mM histidine buffer system providing a composition pH of 5.4-7.1; and 2-25 mM methionine.
N116. 10-60 mg/mL EGFR/LGR5 BsAb; 3-25 mM histidine buffer system providing a composition pH of 5.4-7.1; and 2-25 mM methionine.
N117. 1-150 mg/mL EGFR/LGR5 BsAb; 3-25 mM acetate buffer system providing a composition pH of 4-5.6; and 2-25 mM methionine.
N118. 10-60 mg/mL EGFR/LGR5 BsAb; 3-25 mM acetate buffer system providing a composition pH of 4-5.6; and 2-25 mM methionine.
N119. 1-150 mg/mL EGFR/LGR5 BsAb; 3-25 mM citrate buffer system providing a composition pH of 4.5-7.5; and 2-25 mM methionine.
N120. 10-60 mg/mL EGFR/LGR5 BsAb; 3-25 mM citrate buffer system providing a composition pH of 4.5-7.5; and 2-25 mM methionine.
N121. 1-150 mg/mL EGFR/LGR5 BsAb; 3-25 mM succinate buffer system providing a composition pH of 4-6.6; and 2-25 mM methionine.
N122. 10-60 mg/mL EGFR/LGR5 BsAb; 3-25 mM succinate buffer system providing a composition pH of 4-6.6; and 2-25 mM methionine.
N123. 1-150 mg/mL EGFR/LGR5 BsAb; 3-25 mM phosphate buffer system providing a composition pH of 5.8-8.1; and 2-25 mM methionine.
N124. 10-60 mg/mL EGFR/LGR5 BsAb; 3-25 mM phosphate buffer system providing a composition pH of 5.8-8.1; and 2-25 mM methionine.
N125. 1-150 mg/mL EGFR/LGR5 BsAb; 3-25 mM citrate-phosphate buffer system providing a composition pH of 4-7.1; and 2-25 mM methionine.
N126. 10-60 mg/mL EGFR/LGR5 BsAb; 3-25 mM citrate-phosphate buffer system providing a composition pH of 4-7.1; and 2-25 mM methionine.
N127. 1-150 mg/mL EGFR/LGR5 BsAb; 3-25 mM histidine buffer system providing a composition pH of 5.4-7.1; and 0.01-0.2 mM EDTA.
N128. 10-60 mg/mL EGFR/LGR5 BsAb; 3-25 mM histidine buffer system providing a composition pH of 5.4-7.1; and 0.01-0.2 mM EDTA.
N129. 1-150 mg/mL EGFR/LGR5 BsAb; 3-25 mM acetate buffer system providing a composition pH of 4-5.6; and 0.01-0.2 mM EDTA.
N130. 10-60 mg/mL EGFR/LGR5 BsAb; 3-25 mM acetate buffer system providing a composition pH of 4-5.6; and 0.01-0.2 mM EDTA.
N131. 1-150 mg/mL EGFR/LGR5 BsAb; 3-25 mM citrate buffer system providing a composition pH of 4.5-7.5; and 0.01-0.2 mM EDTA.
N132. 10-60 mg/mL EGFR/LGR5 BsAb; 3-25 mM citrate buffer system providing a composition pH of 4.5-7.5; and 0.01-0.2 mM EDTA.
N133. 1-150 mg/mL EGFR/LGR5 BsAb; 3-25 mM succinate buffer system providing a composition pH of 4-6.6; and 0.01-0.2 mM EDTA.
N134. 10-60 mg/mL EGFR/LGR5 BsAb; 3-25 mM succinate buffer system providing a composition pH of 4-6.6; and 0.01-0.2 mM EDTA.
N135. 1-150 mg/mL EGFR/LGR5 BsAb; 3-25 mM phosphate buffer system providing a composition pH of 5.8-8.1; and 0.01-0.2 mM EDTA.
N136. 10-60 mg/mL EGFR/LGR5 BsAb; 3-25 mM phosphate buffer system providing a composition pH of 5.8-8.1; and 0.01-0.2 mM EDTA.
N137. 1-150 mg/mL EGFR/LGR5 BsAb; 3-25 mM citrate-phosphate buffer system providing a composition pH of 4-7.1; and 0.01-0.2 mM EDTA.
N138. 10-60 mg/mL EGFR/LGR5 BsAb; 3-25 mM citrate-phosphate buffer system providing a composition pH of 4-7.1; and 0.01-0.2 mM EDTA.
N139. 1-150 mg/mL EGFR/LGR5 BsAb; 10-180 mM sodium chloride; and 100-400 mM sugar component selected from the group consisting of sucrose, mannitol, sorbitol, and maltose.
N140. 10-60 mg/mL EGFR/LGR5 BsAb; 10-180 mM sodium chloride; and 100-400 mM sugar component selected from the group consisting of sucrose, mannitol, sorbitol, and maltose.
N141. 1-150 mg/mL EGFR/LGR5 BsAb; 10-180 mM sodium chloride; and 150-350 mM sucrose.
N142. 10-60 mg/mL EGFR/LGR5 BsAb; 10-180 mM sodium chloride; and 150-350 mM sucrose.
N143. 1-150 mg/mL EGFR/LGR5 BsAb; 10-180 mM sodium chloride; and 150-350 mM maltose.
N144. 10-60 mg/mL EGFR/LGR5 BsAb; 10-180 mM sodium chloride; and 150-350 mM maltose.
N145. 1-150 mg/mL EGFR/LGR5 BsAb; 10-180 mM sodium chloride; and 0.4-1.2 mg/mL surfactant.
N146. 10-60 mg/mL EGFR/LGR5 BsAb; 10-180 mM sodium chloride; and 0.4-1.2 mg/mL surfactant.
N147. 1-150 mg/mL EGFR/LGR5 BsAb; 10-180 mM sodium chloride; and 0.1-1.1 mg/mL polysorbate 80.
N148. 10-60 mg/mL EGFR/LGR5 BsAb; 10-180 mM sodium chloride; and 0.1-1.1 mg/mL polysorbate 80.
N149. 1-150 mg/mL EGFR/LGR5 BsAb; 10-180 mM sodium chloride; and 5-150 mM amino acid component selected from the group consisting of proline, glycine, lysine, and any combination thereof.
N150. 10-60 mg/mL EGFR/LGR5 BsAb; 10-180 mM sodium chloride; and 5-150 mM amino acid component selected from the group consisting of proline, glycine, lysine, and any combination thereof.
N151. 1-150 mg/mL EGFR/LGR5 BsAb; 10-180 mM sodium chloride; and 2-25 mM methionine.
N152. 10-60 mg/mL EGFR/LGR5 BsAb; 10-180 mM sodium chloride; and 2-25 mM methionine.
N153. 1-150 mg/mL EGFR/LGR5 BsAb; 10-180 mM sodium chloride; and 0.01-0.2 mM EDTA.
N154. 10-60 mg/mL EGFR/LGR5 BsAb; 10-180 mM sodium chloride; and 0.01-0.2 mM EDTA.
N155. 1-150 mg/mL EGFR/LGR5 BsAb; 100-400 mM sugar component selected from the group consisting of sucrose, mannitol, sorbitol, and maltose; and 0.4-1.2 mg/mL surfactant.
N156. 10-60 mg/mL EGFR/LGR5 BsAb; 100-400 mM sugar component selected from the group consisting of sucrose, mannitol, sorbitol, and maltose; and 0.4-1.2 mg/mL surfactant.
N157. 1-150 mg/mL EGFR/LGR5 BsAb; 150-350 mM sucrose; and 0.4-1.2 mg/mL surfactant.
N158. 10-60 mg/mL EGFR/LGR5 BsAb; 150-350 mM sucrose; and 0.4-1.2 mg/mL surfactant.
N159. 1-150 mg/mL EGFR/LGR5 BsAb; 150-350 mM maltose; and 0.4-1.2 mg/mL surfactant.
N160. 10-60 mg/mL EGFR/LGR5 BsAb; 150-350 mM maltose; and 0.4-1.2 mg/mL surfactant.
N161. 1-150 mg/mL EGFR/LGR5 BsAb; 100-400 mM sugar component selected from the group consisting of sucrose, mannitol, sorbitol, and maltose; and 0.1-1.1 mg/mL polysorbate 80.
N162. 10-60 mg/mL EGFR/LGR5 BsAb; 100-400 mM sugar component selected from the group consisting of sucrose, mannitol, sorbitol, and maltose; and 0.1-1.1 mg/mL polysorbate 80.
N163. 1-150 mg/mL EGFR/LGR5 BsAb; 150-350 mM sucrose; and 0.1-1.1 mg/mL polysorbate 80.
N164. 10-60 mg/mL EGFR/LGR5 BsAb; 150-350 mM sucrose; and 0.1-1.1 mg/mL polysorbate 80.
N165. 1-150 mg/mL EGFR/LGR5 BsAb; 150-350 mM maltose; and 0.1-1.1 mg/mL polysorbate 80.
N166. 10-60 mg/mL EGFR/LGR5 BsAb; 150-350 mM maltose; and 0.1-1.1 mg/mL polysorbate 80.
N167. 1-150 mg/mL EGFR/LGR5 BsAb; 100-400 mM sugar component selected from the group consisting of sucrose, mannitol, sorbitol, and maltose; and 5-150 mM amino acid component selected from the group consisting of proline, glycine, lysine, and any combination thereof.
N168. 10-60 mg/mL EGFR/LGR5 BsAb; 100-400 mM sugar component selected from the group consisting of sucrose, mannitol, sorbitol, and maltose; and 5-150 mM amino acid component selected from the group consisting of proline, glycine, lysine, and any combination thereof.
N169. 1-150 mg/mL EGFR/LGR5 BsAb; 150-350 mM sucrose; and 5-150 mM amino acid component selected from the group consisting of proline, glycine, lysine, and any combination thereof.
N170. 10-60 mg/mL EGFR/LGR5 BsAb; 150-350 mM sucrose; and 5-150 mM amino acid component selected from the group consisting of proline, glycine, lysine, and any combination thereof.
N171. 1-150 mg/mL EGFR/LGR5 BsAb; 150-350 mM maltose; and 5-150 mM amino acid component selected from the group consisting of proline, glycine, lysine, and any combination thereof.
N172. 10-60 mg/mL EGFR/LGR5 BsAb; 150-350 mM maltose; and 5-150 mM amino acid component selected from the group consisting of proline, glycine, lysine, and any combination thereof.
N173. 1-150 mg/mL EGFR/LGR5 BsAb; 100-400 mM sugar component selected from the group consisting of sucrose, mannitol, sorbitol, and maltose; and 2-25 mM methionine.
N174. 10-60 mg/mL EGFR/LGR5 BsAb; 100-400 mM sugar component selected from the group consisting of sucrose, mannitol, sorbitol, and maltose; and 2-25 mM methionine.
N175. 1-150 mg/mL EGFR/LGR5 BsAb; 150-350 mM sucrose; and 2-25 mM methionine.
N176. 10-60 mg/mL EGFR/LGR5 BsAb; 150-350 mM sucrose; and 2-25 mM methionine.
N177. 1-150 mg/mL EGFR/LGR5 BsAb; 150-350 mM maltose; and 2-25 mM methionine.
N178. 10-60 mg/mL EGFR/LGR5 BsAb; 150-350 mM maltose; and 2-25 mM methionine.
N179. 1-150 mg/mL EGFR/LGR5 BsAb; 100-400 mM sugar component selected from the group consisting of sucrose, mannitol, sorbitol, and maltose; and 0.01-0.2 mM EDTA.
N180. 10-60 mg/mL EGFR/LGR5 BsAb; 100-400 mM sugar component selected from the group consisting of sucrose, mannitol, sorbitol, and maltose; and 0.01-0.2 mM EDTA.
N181. 1-150 mg/mL EGFR/LGR5 BsAb; 150-350 mM sucrose; and 0.01-0.2 mM EDTA.
N182. 10-60 mg/mL EGFR/LGR5 BsAb; 150-350 mM sucrose; and 0.01-0.2 mM EDTA.
N183. 1-150 mg/mL EGFR/LGR5 BsAb; 150-350 mM maltose; and 0.01-0.2 mM EDTA.
N184. 10-60 mg/mL EGFR/LGR5 BsAb; 150-350 mM maltose; and 0.01-0.2 mM EDTA.
N185. 1-150 mg/mL EGFR/LGR5 BsAb; 0.4-1.2 mg/mL surfactant; and 5-150 mM amino acid component selected from the group consisting of proline, glycine, lysine, and any combination thereof.
N186. 10-60 mg/mL EGFR/LGR5 BsAb; 0.4-1.2 mg/mL surfactant; and 5-150 mM amino acid component selected from the group consisting of proline, glycine, lysine, and any combination thereof.
N187. 1-150 mg/mL EGFR/LGR5 BsAb; 0.1-1.1 mg/mL polysorbate 80; and 5-150 mM amino acid component selected from the group consisting of proline, glycine, lysine, and any combination thereof.
N188. 10-60 mg/mL EGFR/LGR5 BsAb; 0.1-1.1 mg/mL polysorbate 80; and 5-150 mM amino acid component selected from the group consisting of proline, glycine, lysine, and any combination thereof.
N189. 1-150 mg/mL EGFR/LGR5 BsAb; 0.4-1.2 mg/mL surfactant; and 2-25 mM methionine.
N190. 10-60 mg/mL EGFR/LGR5 BsAb; 0.4-1.2 mg/mL surfactant; and 2-25 mM methionine.
N191. 1-150 mg/mL EGFR/LGR5 BsAb; 0.1-1.1 mg/mL polysorbate 80; and 2-25 mM methionine.
N192. 10-60 mg/mL EGFR/LGR5 BsAb; 0.1-1.1 mg/mL polysorbate 80; and 2-25 mM methionine.
N193. 1-150 mg/mL EGFR/LGR5 BsAb; 0.4-1.2 mg/mL surfactant; and 0.01-0.2 mM EDTA.
N194. 10-60 mg/mL EGFR/LGR5 BsAb; 0.4-1.2 mg/mL surfactant; and 0.01-0.2 mM EDTA.
N195. 1-150 mg/mL EGFR/LGR5 BsAb; 0.1-1.1 mg/mL polysorbate 80; and 0.01-0.2 mM EDTA.
N196. 10-60 mg/mL EGFR/LGR5 BsAb; 0.1-1.1 mg/mL polysorbate 80; and 0.01-0.2 mM EDTA.
N197. 1-150 mg/mL EGFR/LGR5 BsAb; 5-150 mM amino acid component selected from the group consisting of proline, glycine, lysine, and any combination thereof; and 2-25 mM methionine.
N198. 10-60 mg/mL EGFR/LGR5 BsAb; 5-150 mM amino acid component selected from the group consisting of proline, glycine, lysine, and any combination thereof; and 2-25 mM methionine.
N199. 1-150 mg/mL EGFR/LGR5 BsAb; 5-150 mM amino acid component selected from the group consisting of proline, glycine, lysine, and any combination thereof; and 0.01-0.2 mM EDTA.
N200. 10-60 mg/mL EGFR/LGR5 BsAb; 5-150 mM amino acid component selected from the group consisting of proline, glycine, lysine, and any combination thereof; and 0.01-0.2 mM EDTA.
N201. 1-150 mg/mL EGFR/LGR5 BsAb; 2-25 mM methionine; and 0.01-0.2 mM EDTA.
N202. 10-60 mg/mL EGFR/LGR5 BsAb; 2-25 mM methionine; and 0.01-0.2 mM EDTA.
N203. 1-150 mg/mL EGFR/LGR5 BsAb; 3-25 mM histidine buffer system providing a composition pH of 5.4-7.1; 10-180 mM sodium chloride; and 100-400 mM sugar component selected from the group consisting of sucrose, mannitol, sorbitol, and maltose.
N204. 10-60 mg/mL EGFR/LGR5 BsAb; 3-25 mM histidine buffer system providing a composition pH of 5.4-7.1; 10-180 mM sodium chloride; and 100-400 mM sugar component selected from the group consisting of sucrose, mannitol, sorbitol, and maltose.
N205. 1-150 mg/mL EGFR/LGR5 BsAb; 3-25 mM acetate buffer system providing a composition pH of 4-5.6; 10-180 mM sodium chloride; and 100-400 mM sugar component selected from the group consisting of sucrose, mannitol, sorbitol, and maltose.
N206. 10-60 mg/mL EGFR/LGR5 BsAb; 3-25 mM acetate buffer system providing a composition pH of 4-5.6; 10-180 mM sodium chloride; and 100-400 mM sugar component selected from the group consisting of sucrose, mannitol, sorbitol, and maltose.
N207. 1-150 mg/mL EGFR/LGR5 BsAb; 3-25 mM citrate buffer system providing a composition pH of 4.5-7.5; 10-180 mM sodium chloride; and 100-400 mM sugar component selected from the group consisting of sucrose, mannitol, sorbitol, and maltose.
N208. 10-60 mg/mL EGFR/LGR5 BsAb; 3-25 mM citrate buffer system providing a composition pH of 4.5-7.5; 10-180 mM sodium chloride; and 100-400 mM sugar component selected from the group consisting of sucrose, mannitol, sorbitol, and maltose.
N209. 1-150 mg/mL EGFR/LGR5 BsAb; 3-25 mM succinate buffer system providing a composition pH of 4-6.6; 10-180 mM sodium chloride; and 100-400 mM sugar component selected from the group consisting of sucrose, mannitol, sorbitol, and maltose.
N210. 10-60 mg/mL EGFR/LGR5 BsAb; 3-25 mM succinate buffer system providing a composition pH of 4-6.6; 10-180 mM sodium chloride; and 100-400 mM sugar component selected from the group consisting of sucrose, mannitol, sorbitol, and maltose.
N211. 1-150 mg/mL EGFR/LGR5 BsAb; 3-25 mM phosphate buffer system providing a composition pH of 5.8-8.1; 10-180 mM sodium chloride; and 100-400 mM sugar component selected from the group consisting of sucrose, mannitol, sorbitol, and maltose.
N212. 10-60 mg/mL EGFR/LGR5 BsAb; 3-25 mM phosphate buffer system providing a composition pH of 5.8-8.1; 10-180 mM sodium chloride; and 100-400 mM sugar component selected from the group consisting of sucrose, mannitol, sorbitol, and maltose.
N213. 1-150 mg/mL EGFR/LGR5 BsAb; 3-25 mM citrate-phosphate buffer system providing a composition pH of 4-7.1; 10-180 mM sodium chloride; and 100-400 mM sugar component selected from the group consisting of sucrose, mannitol, sorbitol, and maltose.
N214. 10-60 mg/mL EGFR/LGR5 BsAb; 3-25 mM citrate-phosphate buffer system providing a composition pH of 4-7.1; 10-180 mM sodium chloride; and 100-400 mM sugar component selected from the group consisting of sucrose, mannitol, sorbitol, and maltose.
N215. 1-150 mg/mL EGFR/LGR5 BsAb; 3-25 mM histidine buffer system providing a composition pH of 5.4-7.1; 10-180 mM sodium chloride; and 150-350 mM sucrose.
N216. 10-60 mg/mL EGFR/LGR5 BsAb; 3-25 mM histidine buffer system providing a composition pH of 5.4-7.1; 10-180 mM sodium chloride; and 150-350 mM sucrose.
N217. 1-150 mg/mL EGFR/LGR5 BsAb; 3-25 mM acetate buffer system providing a composition pH of 4-5.6; 10-180 mM sodium chloride; and 150-350 mM sucrose.
N218. 10-60 mg/mL EGFR/LGR5 BsAb; 3-25 mM acetate buffer system providing a composition pH of 4-5.6; 10-180 mM sodium chloride; and 150-350 mM sucrose.
N219. 1-150 mg/mL EGFR/LGR5 BsAb; 3-25 mM citrate buffer system providing a composition pH of 4.5-7.5; 10-180 mM sodium chloride; and 150-350 mM sucrose.
N220. 10-60 mg/mL EGFR/LGR5 BsAb; 3-25 mM citrate buffer system providing a composition pH of 4.5-7.5; 10-180 mM sodium chloride; and 150-350 mM sucrose.
N221. 1-150 mg/mL EGFR/LGR5 BsAb; 3-25 mM succinate buffer system providing a composition pH of 4-6.6; 10-180 mM sodium chloride; and 150-350 mM sucrose.
N222. 10-60 mg/mL EGFR/LGR5 BsAb; 3-25 mM succinate buffer system providing a composition pH of 4-6.6; 10-180 mM sodium chloride; and 150-350 mM sucrose.
N223. 1-150 mg/mL EGFR/LGR5 BsAb; 3-25 mM phosphate buffer system providing a composition pH of 5.8-8.1; 10-180 mM sodium chloride; and 150-350 mM sucrose.
N224. 10-60 mg/mL EGFR/LGR5 BsAb; 3-25 mM phosphate buffer system providing a composition pH of 5.8-8.1; 10-180 mM sodium chloride; and 150-350 mM sucrose.
N225. 1-150 mg/mL EGFR/LGR5 BsAb; 3-25 mM citrate-phosphate buffer system providing a composition pH of 4-7.1; 10-180 mM sodium chloride; and 150-350 mM sucrose.
N226. 10-60 mg/mL EGFR/LGR5 BsAb; 3-25 mM citrate-phosphate buffer system providing a composition pH of 4-7.1; 10-180 mM sodium chloride; and 150-350 mM sucrose.
N227. 1-150 mg/mL EGFR/LGR5 BsAb; 3-25 mM histidine buffer system providing a composition pH of 5.4-7.1; 10-180 mM sodium chloride; and 150-350 mM maltose.
N228. 10-60 mg/mL EGFR/LGR5 BsAb; 3-25 mM histidine buffer system providing a composition pH of 5.4-7.1; 10-180 mM sodium chloride; and 150-350 mM maltose.
N229. 1-150 mg/mL EGFR/LGR5 BsAb; 3-25 mM acetate buffer system providing a composition pH of 4-5.6; 10-180 mM sodium chloride; and 150-350 mM maltose.
N230. 10-60 mg/mL EGFR/LGR5 BsAb; 3-25 mM acetate buffer system providing a composition pH of 4-5.6; 10-180 mM sodium chloride; and 150-350 mM maltose.
N231. 1-150 mg/mL EGFR/LGR5 BsAb; 3-25 mM citrate buffer system providing a composition pH of 4.5-7.5; 10-180 mM sodium chloride; and 150-350 mM maltose.
N232. 10-60 mg/mL EGFR/LGR5 BsAb; 3-25 mM citrate buffer system providing a composition pH of 4.5-7.5; 10-180 mM sodium chloride; and 150-350 mM maltose.
N233. 1-150 mg/mL EGFR/LGR5 BsAb; 3-25 mM succinate buffer system providing a composition pH of 4-6.6; 10-180 mM sodium chloride; and 150-350 mM maltose.
N234. 10-60 mg/mL EGFR/LGR5 BsAb; 3-25 mM succinate buffer system providing a composition pH of 4-6.6; 10-180 mM sodium chloride; and 150-350 mM maltose.
N235. 1-150 mg/mL EGFR/LGR5 BsAb; 3-25 mM phosphate buffer system providing a composition pH of 5.8-8.1; 10-180 mM sodium chloride; and 150-350 mM maltose.
N236. 10-60 mg/mL EGFR/LGR5 BsAb; 3-25 mM phosphate buffer system providing a composition pH of 5.8-8.1; 10-180 mM sodium chloride; and 150-350 mM maltose.
N237. 1-150 mg/mL EGFR/LGR5 BsAb; 3-25 mM citrate-phosphate buffer system providing a composition pH of 4-7.1; 10-180 mM sodium chloride; and 150-350 mM maltose.
N238. 10-60 mg/mL EGFR/LGR5 BsAb; 3-25 mM citrate-phosphate buffer system providing a composition pH of 4-7.1; 10-180 mM sodium chloride; and 150-350 mM maltose.
N239. 1-150 mg/mL EGFR/LGR5 BsAb; 3-25 mM histidine buffer system providing a composition pH of 5.4-7.1; 10-180 mM sodium chloride; and 0.4-1.2 mg/mL surfactant.
N240. 10-60 mg/mL EGFR/LGR5 BsAb; 3-25 mM histidine buffer system providing a composition pH of 5.4-7.1; 10-180 mM sodium chloride; and 0.4-1.2 mg/mL surfactant.
N241. 1-150 mg/mL EGFR/LGR5 BsAb; 3-25 mM acetate buffer system providing a composition pH of 4-5.6; 10-180 mM sodium chloride; and 0.4-1.2 mg/mL surfactant.
N242. 10-60 mg/mL EGFR/LGR5 BsAb; 3-25 mM acetate buffer system providing a composition pH of 4-5.6; 10-180 mM sodium chloride; and 0.4-1.2 mg/mL surfactant.
N243. 1-150 mg/mL EGFR/LGR5 BsAb; 3-25 mM citrate buffer system providing a composition pH of 4.5-7.5; 10-180 mM sodium chloride; and 0.4-1.2 mg/mL surfactant.
N244. 10-60 mg/mL EGFR/LGR5 BsAb; 3-25 mM citrate buffer system providing a composition pH of 4.5-7.5; 10-180 mM sodium chloride; and 0.4-1.2 mg/mL surfactant.
N245. 1-150 mg/mL EGFR/LGR5 BsAb; 3-25 mM succinate buffer system providing a composition pH of 4-6.6; 10-180 mM sodium chloride; and 0.4-1.2 mg/mL surfactant.
N246. 10-60 mg/mL EGFR/LGR5 BsAb; 3-25 mM succinate buffer system providing a composition pH of 4-6.6; 10-180 mM sodium chloride; and 0.4-1.2 mg/mL surfactant.
N247. 1-150 mg/mL EGFR/LGR5 BsAb; 3-25 mM phosphate buffer system providing a composition pH of 5.8-8.1; 10-180 mM sodium chloride; and 0.4-1.2 mg/mL surfactant.
N248. 10-60 mg/mL EGFR/LGR5 BsAb; 3-25 mM phosphate buffer system providing a composition pH of 5.8-8.1; 10-180 mM sodium chloride; and 0.4-1.2 mg/mL surfactant.
N249. 1-150 mg/mL EGFR/LGR5 BsAb; 3-25 mM citrate-phosphate buffer system providing a composition pH of 4-7.1; 10-180 mM sodium chloride; and 0.4-1.2 mg/mL surfactant.
N250. 10-60 mg/mL EGFR/LGR5 BsAb; 3-25 mM citrate-phosphate buffer system providing a composition pH of 4-7.1; 10-180 mM sodium chloride; and 0.4-1.2 mg/mL surfactant.
N251. 1-150 mg/mL EGFR/LGR5 BsAb; 3-25 mM histidine buffer system providing a composition pH of 5.4-7.1; 10-180 mM sodium chloride; and 0.1-1.1 mg/mL polysorbate 80.
N252. 10-60 mg/mL EGFR/LGR5 BsAb; 3-25 mM histidine buffer system providing a composition pH of 5.4-7.1; 10-180 mM sodium chloride; and 0.1-1.1 mg/mL polysorbate 80.
N253. 1-150 mg/mL EGFR/LGR5 BsAb; 3-25 mM acetate buffer system providing a composition pH of 4-5.6; 10-180 mM sodium chloride; and 0.1-1.1 mg/mL polysorbate 80.
N254. 10-60 mg/mL EGFR/LGR5 BsAb; 3-25 mM acetate buffer system providing a composition pH of 4-5.6; 10-180 mM sodium chloride; and 0.1-1.1 mg/mL polysorbate 80.
N255. 1-150 mg/mL EGFR/LGR5 BsAb; 3-25 mM citrate buffer system providing a composition pH of 4.5-7.5; 10-180 mM sodium chloride; and 0.1-1.1 mg/mL polysorbate 80.
N256. 10-60 mg/mL EGFR/LGR5 BsAb; 3-25 mM citrate buffer system providing a composition pH of 4.5-7.5; 10-180 mM sodium chloride; and 0.1-1.1 mg/mL polysorbate 80.
N257. 1-150 mg/mL EGFR/LGR5 BsAb; 3-25 mM succinate buffer system providing a composition pH of 4-6.6; 10-180 mM sodium chloride; and 0.1-1.1 mg/mL polysorbate 80.
N258. 10-60 mg/mL EGFR/LGR5 BsAb; 3-25 mM succinate buffer system providing a composition pH of 4-6.6; 10-180 mM sodium chloride; and 0.1-1.1 mg/mL polysorbate 80.
N259. 1-150 mg/mL EGFR/LGR5 BsAb; 3-25 mM phosphate buffer system providing a composition pH of 5.8-8.1; 10-180 mM sodium chloride; and 0.1-1.1 mg/mL polysorbate 80.
N260. 10-60 mg/mL EGFR/LGR5 BsAb; 3-25 mM phosphate buffer system providing a composition pH of 5.8-8.1; 10-180 mM sodium chloride; and 0.1-1.1 mg/mL polysorbate 80.
N261. 1-150 mg/mL EGFR/LGR5 BsAb; 3-25 mM citrate-phosphate buffer system providing a composition pH of 4-7.1; 10-180 mM sodium chloride; and 0.1-1.1 mg/mL polysorbate 80.
N262. 10-60 mg/mL EGFR/LGR5 BsAb; 3-25 mM citrate-phosphate buffer system providing a composition pH of 4-7.1; 10-180 mM sodium chloride; and 0.1-1.1 mg/mL polysorbate 80.
N263. 1-150 mg/mL EGFR/LGR5 BsAb; 3-25 mM histidine buffer system providing a composition pH of 5.4-7.1; 10-180 mM sodium chloride; and 5-150 mM amino acid component selected from the group consisting of proline, glycine, lysine, and any combination thereof.
N264. 10-60 mg/mL EGFR/LGR5 BsAb; 3-25 mM histidine buffer system providing a composition pH of 5.4-7.1; 10-180 mM sodium chloride; and 5-150 mM amino acid component selected from the group consisting of proline, glycine, lysine, and any combination thereof.
N265. 1-150 mg/mL EGFR/LGR5 BsAb; 3-25 mM acetate buffer system providing a composition pH of 4-5.6; 10-180 mM sodium chloride; and 5-150 mM amino acid component selected from the group consisting of proline, glycine, lysine, and any combination thereof.
N266. 10-60 mg/mL EGFR/LGR5 BsAb; 3-25 mM acetate buffer system providing a composition pH of 4-5.6; 10-180 mM sodium chloride; and 5-150 mM amino acid component selected from the group consisting of proline, glycine, lysine, and any combination thereof.
N267. 1-150 mg/mL EGFR/LGR5 BsAb; 3-25 mM citrate buffer system providing a composition pH of 4.5-7.5; 10-180 mM sodium chloride; and 5-150 mM amino acid component selected from the group consisting of proline, glycine, lysine, and any combination thereof.
N268. 10-60 mg/mL EGFR/LGR5 BsAb; 3-25 mM citrate buffer system providing a composition pH of 4.5-7.5; 10-180 mM sodium chloride; and 5-150 mM amino acid component selected from the group consisting of proline, glycine, lysine, and any combination thereof.
N269. 1-150 mg/mL EGFR/LGR5 BsAb; 3-25 mM succinate buffer system providing a composition pH of 4-6.6; 10-180 mM sodium chloride; and 5-150 mM amino acid component selected from the group consisting of proline, glycine, lysine, and any combination thereof.
N270. 10-60 mg/mL EGFR/LGR5 BsAb; 3-25 mM succinate buffer system providing a composition pH of 4-6.6; 10-180 mM sodium chloride; and 5-150 mM amino acid component selected from the group consisting of proline, glycine, lysine, and any combination thereof.
N271. 1-150 mg/mL EGFR/LGR5 BsAb; 3-25 mM phosphate buffer system providing a composition pH of 5.8-8.1; 10-180 mM sodium chloride; and 5-150 mM amino acid component selected from the group consisting of proline, glycine, lysine, and any combination thereof.
N272. 10-60 mg/mL EGFR/LGR5 BsAb; 3-25 mM phosphate buffer system providing a composition pH of 5.8-8.1; 10-180 mM sodium chloride; and 5-150 mM amino acid component selected from the group consisting of proline, glycine, lysine, and any combination thereof.
N273. 1-150 mg/mL EGFR/LGR5 BsAb; 3-25 mM citrate-phosphate buffer system providing a composition pH of 4-7.1; 10-180 mM sodium chloride; and 5-150 mM amino acid component selected from the group consisting of proline, glycine, lysine, and any combination thereof.
N274. 10-60 mg/mL EGFR/LGR5 BsAb; 3-25 mM citrate-phosphate buffer system providing a composition pH of 4-7.1; 10-180 mM sodium chloride; and 5-150 mM amino acid component selected from the group consisting of proline, glycine, lysine, and any combination thereof.
N275. 1-150 mg/mL EGFR/LGR5 BsAb; 3-25 mM histidine buffer system providing a composition pH of 5.4-7.1; 10-180 mM sodium chloride; and 2-25 mM methionine.
N276. 10-60 mg/mL EGFR/LGR5 BsAb; 3-25 mM histidine buffer system providing a composition pH of 5.4-7.1; 10-180 mM sodium chloride; and 2-25 mM methionine.
N277. 1-150 mg/mL EGFR/LGR5 BsAb; 3-25 mM acetate buffer system providing a composition pH of 4-5.6; 10-180 mM sodium chloride; and 2-25 mM methionine.
N278. 10-60 mg/mL EGFR/LGR5 BsAb; 3-25 mM acetate buffer system providing a composition pH of 4-5.6; 10-180 mM sodium chloride; and 2-25 mM methionine.
N279. 1-150 mg/mL EGFR/LGR5 BsAb; 3-25 mM citrate buffer system providing a composition pH of 4.5-7.5; 10-180 mM sodium chloride; and 2-25 mM methionine.
N280. 10-60 mg/mL EGFR/LGR5 BsAb; 3-25 mM citrate buffer system providing a composition pH of 4.5-7.5; 10-180 mM sodium chloride; and 2-25 mM methionine.
N281. 1-150 mg/mL EGFR/LGR5 BsAb; 3-25 mM succinate buffer system providing a composition pH of 4-6.6; 10-180 mM sodium chloride; and 2-25 mM methionine.
N282. 10-60 mg/mL EGFR/LGR5 BsAb; 3-25 mM succinate buffer system providing a composition pH of 4-6.6; 10-180 mM sodium chloride; and 2-25 mM methionine.
N283. 1-150 mg/mL EGFR/LGR5 BsAb; 3-25 mM phosphate buffer system providing a composition pH of 5.8-8.1; 10-180 mM sodium chloride; and 2-25 mM methionine.
N284. 10-60 mg/mL EGFR/LGR5 BsAb; 3-25 mM phosphate buffer system providing a composition pH of 5.8-8.1; 10-180 mM sodium chloride; and 2-25 mM methionine.
N285. 1-150 mg/mL EGFR/LGR5 BsAb; 3-25 mM citrate-phosphate buffer system providing a composition pH of 4-7.1; 10-180 mM sodium chloride; and 2-25 mM methionine.
N286. 10-60 mg/mL EGFR/LGR5 BsAb; 3-25 mM citrate-phosphate buffer system providing a composition pH of 4-7.1; 10-180 mM sodium chloride; and 2-25 mM methionine.
N287. 1-150 mg/mL EGFR/LGR5 BsAb; 3-25 mM histidine buffer system providing a composition pH of 5.4-7.1; 10-180 mM sodium chloride; and 0.01-0.2 mM EDTA.
N288. 10-60 mg/mL EGFR/LGR5 BsAb; 3-25 mM histidine buffer system providing a composition pH of 5.4-7.1; 10-180 mM sodium chloride; and 0.01-0.2 mM EDTA.
N289. 1-150 mg/mL EGFR/LGR5 BsAb; 3-25 mM acetate buffer system providing a composition pH of 4-5.6; 10-180 mM sodium chloride; and 0.01-0.2 mM EDTA.
N290. 10-60 mg/mL EGFR/LGR5 BsAb; 3-25 mM acetate buffer system providing a composition pH of 4-5.6; 10-180 mM sodium chloride; and 0.01-0.2 mM EDTA.
N291. 1-150 mg/mL EGFR/LGR5 BsAb; 3-25 mM citrate buffer system providing a composition pH of 4.5-7.5; 10-180 mM sodium chloride; and 0.01-0.2 mM EDTA.
N292. 10-60 mg/mL EGFR/LGR5 BsAb; 3-25 mM citrate buffer system providing a composition pH of 4.5-7.5; 10-180 mM sodium chloride; and 0.01-0.2 mM EDTA.
N293. 1-150 mg/mL EGFR/LGR5 BsAb; 3-25 mM succinate buffer system providing a composition pH of 4-6.6; 10-180 mM sodium chloride; and 0.01-0.2 mM EDTA.
N294. 10-60 mg/mL EGFR/LGR5 BsAb; 3-25 mM succinate buffer system providing a composition pH of 4-6.6; 10-180 mM sodium chloride; and 0.01-0.2 mM EDTA.
N295. 1-150 mg/mL EGFR/LGR5 BsAb; 3-25 mM phosphate buffer system providing a composition pH of 5.8-8.1; 10-180 mM sodium chloride; and 0.01-0.2 mM EDTA.
N296. 10-60 mg/mL EGFR/LGR5 BsAb; 3-25 mM phosphate buffer system providing a composition pH of 5.8-8.1; 10-180 mM sodium chloride; and 0.01-0.2 mM EDTA.
N297. 1-150 mg/mL EGFR/LGR5 BsAb; 3-25 mM citrate-phosphate buffer system providing a composition pH of 4-7.1; 10-180 mM sodium chloride; and 0.01-0.2 mM EDTA.
N298. 10-60 mg/mL EGFR/LGR5 BsAb; 3-25 mM citrate-phosphate buffer system providing a composition pH of 4-7.1; 10-180 mM sodium chloride; and 0.01-0.2 mM EDTA.
N299. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.4-1.2 mg/mL surfactant.
N300. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.4-1.2 mg/mL surfactant.
N301. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.4-1.2 mg/mL surfactant.
N302. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.4-1.2 mg/mL surfactant.
N303. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.4-1.2 mg/mL surfactant.
N304. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.4-1.2 mg/mL surfactant.
N305. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.4-1.2 mg/mL surfactant.
N306. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.4-1.2 mg/mL surfactant.
N307. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.4-1.2 mg/mL surfactant.
N308. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.4-1.2 mg/mL surfactant.
N309. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.4-1.2 mg/mL surfactant.
N310. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.4-1.2 mg/mL surfactant.
N311. 1-150 mg/mL EGFR/LGR5 BsAb; 3-25 mM histidine buffer system providing a composition pH of 5.4-7.1; 150-350 mM sucrose; and 0.4-1.2 mg/mL surfactant.
N312. 10-60 mg/mL EGFR/LGR5 BsAb; 3-25 mM histidine buffer system providing a composition pH of 5.4-7.1; 150-350 mM sucrose; and 0.4-1.2 mg/mL surfactant.
N313. 1-150 mg/mL EGFR/LGR5 BsAb; 3-25 mM acetate buffer system providing a composition pH of 4-5.6; 150-350 mM sucrose; and 0.4-1.2 mg/mL surfactant.
N314. 10-60 mg/mL EGFR/LGR5 BsAb; 3-25 mM acetate buffer system providing a composition pH of 4-5.6; 150-350 mM sucrose; and 0.4-1.2 mg/mL surfactant.
N315. 1-150 mg/mL EGFR/LGR5 BsAb; 3-25 mM citrate buffer system providing a composition pH of 4.5-7.5; 150-350 mM sucrose; and 0.4-1.2 mg/mL surfactant.
N316. 10-60 mg/mL EGFR/LGR5 BsAb; 3-25 mM citrate buffer system providing a composition pH of 4.5-7.5; 150-350 mM sucrose; and 0.4-1.2 mg/mL surfactant.
N317. 1-150 mg/mL EGFR/LGR5 BsAb; 3-25 mM succinate buffer system providing a composition pH of 4-6.6; 150-350 mM sucrose; and 0.4-1.2 mg/mL surfactant.
N318. 10-60 mg/mL EGFR/LGR5 BsAb; 3-25 mM succinate buffer system providing a composition pH of 4-6.6; 150-350 mM sucrose; and 0.4-1.2 mg/mL surfactant.
N319. 1-150 mg/mL EGFR/LGR5 BsAb; 3-25 mM phosphate buffer system providing a composition pH of 5.8-8.1; 150-350 mM sucrose; and 0.4-1.2 mg/mL surfactant.
N320. 10-60 mg/mL EGFR/LGR5 BsAb; 3-25 mM phosphate buffer system providing a composition pH of 5.8-8.1; 150-350 mM sucrose; and 0.4-1.2 mg/mL surfactant.
N321. 1-150 mg/mL EGFR/LGR5 BsAb; 3-25 mM citrate-phosphate buffer system providing a composition pH of 4-7.1; 150-350 mM sucrose; and 0.4-1.2 mg/mL surfactant.
N322. 10-60 mg/mL EGFR/LGR5 BsAb; 3-25 mM citrate-phosphate buffer system providing a composition pH of 4-7.1; 150-350 mM sucrose; and 0.4-1.2 mg/mL surfactant.
N323. 1-150 mg/mL EGFR/LGR5 BsAb; 3-25 mM histidine buffer system providing a composition pH of 5.4-7.1; 150-350 mM maltose; and 0.4-1.2 mg/mL surfactant.
N324. 10-60 mg/mL EGFR/LGR5 BsAb; 3-25 mM histidine buffer system providing a composition pH of 5.4-7.1; 150-350 mM maltose; and 0.4-1.2 mg/mL surfactant.
N325. 1-150 mg/mL EGFR/LGR5 BsAb; 3-25 mM acetate buffer system providing a composition pH of 4-5.6; 150-350 mM maltose; and 0.4-1.2 mg/mL surfactant.
N326. 10-60 mg/mL EGFR/LGR5 BsAb; 3-25 mM acetate buffer system providing a composition pH of 4-5.6; 150-350 mM maltose; and 0.4-1.2 mg/mL surfactant.
N327. 1-150 mg/mL EGFR/LGR5 BsAb; 3-25 mM citrate buffer system providing a composition pH of 4.5-7.5; 150-350 mM maltose; and 0.4-1.2 mg/mL surfactant.
N328. 10-60 mg/mL EGFR/LGR5 BsAb; 3-25 mM citrate buffer system providing a composition pH of 4.5-7.5; 150-350 mM maltose; and 0.4-1.2 mg/mL surfactant.
N329. 1-150 mg/mL EGFR/LGR5 BsAb; 3-25 mM succinate buffer system providing a composition pH of 4-6.6; 150-350 mM maltose; and 0.4-1.2 mg/mL surfactant.
N330. 10-60 mg/mL EGFR/LGR5 BsAb; 3-25 mM succinate buffer system providing a composition pH of 4-6.6; 150-350 mM maltose; and 0.4-1.2 mg/mL surfactant.
N331. 1-150 mg/mL EGFR/LGR5 BsAb; 3-25 mM phosphate buffer system providing a composition pH of 5.8-8.1; 150-350 mM maltose; and 0.4-1.2 mg/mL surfactant.
N332. 10-60 mg/mL EGFR/LGR5 BsAb; 3-25 mM phosphate buffer system providing a composition pH of 5.8-8.1; 150-350 mM maltose; and 0.4-1.2 mg/mL surfactant.
N333. 1-150 mg/mL EGFR/LGR5 BsAb; 3-25 mM citrate-phosphate buffer system providing a composition pH of 4-7.1; 150-350 mM maltose; and 0.4-1.2 mg/mL surfactant.
N334. 10-60 mg/mL EGFR/LGR5 BsAb; 3-25 mM citrate-phosphate buffer system providing a composition pH of 4-7.1; 150-350 mM maltose; and 0.4-1.2 mg/mL surfactant.
N335. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.1-1.1 mg/mL polysorbate 80.
N336. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.1-1.1 mg/mL polysorbate 80.
N337. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.1-1.1 mg/mL polysorbate 80.
N338. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.1-1.1 mg/mL polysorbate 80.
N339. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.1-1.1 mg/mL polysorbate 80.
N340. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.1-1.1 mg/mL polysorbate 80.
N341. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.1-1.1 mg/mL polysorbate 80.
N342. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.1-1.1 mg/mL polysorbate 80.
N343. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.1-1.1 mg/mL polysorbate 80.
N344. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.1-1.1 mg/mL polysorbate 80.
N345. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.1-1.1 mg/mL polysorbate 80.
N346. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.1-1.1 mg/mL polysorbate 80.
N347. 1-150 mg/mL EGFR/LGR5 BsAb; 3-25 mM histidine buffer system providing a composition pH of 5.4-7.1; 150-350 mM sucrose; and 0.1-1.1 mg/mL polysorbate 80.
N348. 10-60 mg/mL EGFR/LGR5 BsAb; 3-25 mM histidine buffer system providing a composition pH of 5.4-7.1; 150-350 mM sucrose; and 0.1-1.1 mg/mL polysorbate 80.
N349. 1-150 mg/mL EGFR/LGR5 BsAb; 3-25 mM acetate buffer system providing a composition pH of 4-5.6; 150-350 mM sucrose; and 0.1-1.1 mg/mL polysorbate 80.
N350. 10-60 mg/mL EGFR/LGR5 BsAb; 3-25 mM acetate buffer system providing a composition pH of 4-5.6; 150-350 mM sucrose; and 0.1-1.1 mg/mL polysorbate 80.
N351. 1-150 mg/mL EGFR/LGR5 BsAb; 3-25 mM citrate buffer system providing a composition pH of 4.5-7.5; 150-350 mM sucrose; and 0.1-1.1 mg/mL polysorbate 80.
N352. 10-60 mg/mL EGFR/LGR5 BsAb; 3-25 mM citrate buffer system providing a composition pH of 4.5-7.5; 150-350 mM sucrose; and 0.1-1.1 mg/mL polysorbate 80.
N353. 1-150 mg/mL EGFR/LGR5 BsAb; 3-25 mM succinate buffer system providing a composition pH of 4-6.6; 150-350 mM sucrose; and 0.1-1.1 mg/mL polysorbate 80.
N354. 10-60 mg/mL EGFR/LGR5 BsAb; 3-25 mM succinate buffer system providing a composition pH of 4-6.6; 150-350 mM sucrose; and 0.1-1.1 mg/mL polysorbate 80.
N355. 1-150 mg/mL EGFR/LGR5 BsAb; 3-25 mM phosphate buffer system providing a composition pH of 5.8-8.1; 150-350 mM sucrose; and 0.1-1.1 mg/mL polysorbate 80.
N356. 10-60 mg/mL EGFR/LGR5 BsAb; 3-25 mM phosphate buffer system providing a composition pH of 5.8-8.1; 150-350 mM sucrose; and 0.1-1.1 mg/mL polysorbate 80.
N357. 1-150 mg/mL EGFR/LGR5 BsAb; 3-25 mM citrate-phosphate buffer system providing a composition pH of 4-7.1; 150-350 mM sucrose; and 0.1-1.1 mg/mL polysorbate 80.
N358. 10-60 mg/mL EGFR/LGR5 BsAb; 3-25 mM citrate-phosphate buffer system providing a composition pH of 4-7.1; 150-350 mM sucrose; and 0.1-1.1 mg/mL polysorbate 80.
N359. 1-150 mg/mL EGFR/LGR5 BsAb; 3-25 mM histidine buffer system providing a composition pH of 5.4-7.1; 150-350 mM maltose; and 0.1-1.1 mg/mL polysorbate 80.
N360. 10-60 mg/mL EGFR/LGR5 BsAb; 3-25 mM histidine buffer system providing a composition pH of 5.4-7.1; 150-350 mM maltose; and 0.1-1.1 mg/mL polysorbate 80.
N361. 1-150 mg/mL EGFR/LGR5 BsAb; 3-25 mM acetate buffer system providing a composition pH of 4-5.6; 150-350 mM maltose; and 0.1-1.1 mg/mL polysorbate 80.
N362. 10-60 mg/mL EGFR/LGR5 BsAb; 3-25 mM acetate buffer system providing a composition pH of 4-5.6; 150-350 mM maltose; and 0.1-1.1 mg/mL polysorbate 80.
N363. 1-150 mg/mL EGFR/LGR5 BsAb; 3-25 mM citrate buffer system providing a composition pH of 4.5-7.5; 150-350 mM maltose; and 0.1-1.1 mg/mL polysorbate 80.
N364. 10-60 mg/mL EGFR/LGR5 BsAb; 3-25 mM citrate buffer system providing a composition pH of 4.5-7.5; 150-350 mM maltose; and 0.1-1.1 mg/mL polysorbate 80.
N365. 1-150 mg/mL EGFR/LGR5 BsAb; 3-25 mM succinate buffer system providing a composition pH of 4-6.6; 150-350 mM maltose; and 0.1-1.1 mg/mL polysorbate 80.
N366. 10-60 mg/mL EGFR/LGR5 BsAb; 3-25 mM succinate buffer system providing a composition pH of 4-6.6; 150-350 mM maltose; and 0.1-1.1 mg/mL polysorbate 80.
N367. 1-150 mg/mL EGFR/LGR5 BsAb; 3-25 mM phosphate buffer system providing a composition pH of 5.8-8.1; 150-350 mM maltose; and 0.1-1.1 mg/mL polysorbate 80.
N368. 10-60 mg/mL EGFR/LGR5 BsAb; 3-25 mM phosphate buffer system providing a composition pH of 5.8-8.1; 150-350 mM maltose; and 0.1-1.1 mg/mL polysorbate 80.
N369. 1-150 mg/mL EGFR/LGR5 BsAb; 3-25 mM citrate-phosphate buffer system providing a composition pH of 4-7.1; 150-350 mM maltose; and 0.1-1.1 mg/mL polysorbate 80.
N370. 10-60 mg/mL EGFR/LGR5 BsAb; 3-25 mM citrate-phosphate buffer system providing a composition pH of 4-7.1; 150-350 mM maltose; and 0.1-1.1 mg/mL polysorbate 80.
N371. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 5-150 mM amino acid component selected from the group consisting of proline, glycine, lysine, and any combination thereof.
N372. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 5-150 mM amino acid component selected from the group consisting of proline, glycine, lysine, and any combination thereof.
N373. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 5-150 mM amino acid component selected from the group consisting of proline, glycine, lysine, and any combination thereof.
N374. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 5-150 mM amino acid component selected from the group consisting of proline, glycine, lysine, and any combination thereof.
N375. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 5-150 mM amino acid component selected from the group consisting of proline, glycine, lysine, and any combination thereof.
N376. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 5-150 mM amino acid component selected from the group consisting of proline, glycine, lysine, and any combination thereof.
N377. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 5-150 mM amino acid component selected from the group consisting of proline, glycine, lysine, and any combination thereof.
N378. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 5-150 mM amino acid component selected from the group consisting of proline, glycine, lysine, and any combination thereof.
N379. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 5-150 mM amino acid component selected from the group consisting of proline, glycine, lysine, and any combination thereof.
N380. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 5-150 mM amino acid component selected from the group consisting of proline, glycine, lysine, and any combination thereof.
N381. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 5-150 mM amino acid component selected from the group consisting of proline, glycine, lysine, and any combination thereof.
N382. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 5-150 mM amino acid component selected from the group consisting of proline, glycine, lysine, and any combination thereof.
N383. 1-150 mg/mL EGFR/LGR5 BsAb; 3-25 mM histidine buffer system providing a composition pH of 5.4-7.1; 150-350 mM sucrose; and 5-150 mM amino acid component selected from the group consisting of proline, glycine, lysine, and any combination thereof.
N384. 10-60 mg/mL EGFR/LGR5 BsAb; 3-25 mM histidine buffer system providing a composition pH of 5.4-7.1; 150-350 mM sucrose; and 5-150 mM amino acid component selected from the group consisting of proline, glycine, lysine, and any combination thereof.
N385. 1-150 mg/mL EGFR/LGR5 BsAb; 3-25 mM acetate buffer system providing a composition pH of 4-5.6; 150-350 mM sucrose; and 5-150 mM amino acid component selected from the group consisting of proline, glycine, lysine, and any combination thereof.
N386. 10-60 mg/mL EGFR/LGR5 BsAb; 3-25 mM acetate buffer system providing a composition pH of 4-5.6; 150-350 mM sucrose; and 5-150 mM amino acid component selected from the group consisting of proline, glycine, lysine, and any combination thereof.
N387. 1-150 mg/mL EGFR/LGR5 BsAb; 3-25 mM citrate buffer system providing a composition pH of 4.5-7.5; 150-350 mM sucrose; and 5-150 mM amino acid component selected from the group consisting of proline, glycine, lysine, and any combination thereof.
N388. 10-60 mg/mL EGFR/LGR5 BsAb; 3-25 mM citrate buffer system providing a composition pH of 4.5-7.5; 150-350 mM sucrose; and 5-150 mM amino acid component selected from the group consisting of proline, glycine, lysine, and any combination thereof.
N389. 1-150 mg/mL EGFR/LGR5 BsAb; 3-25 mM succinate buffer system providing a composition pH of 4-6.6; 150-350 mM sucrose; and 5-150 mM amino acid component selected from the group consisting of proline, glycine, lysine, and any combination thereof.
N390. 10-60 mg/mL EGFR/LGR5 BsAb; 3-25 mM succinate buffer system providing a composition pH of 4-6.6; 150-350 mM sucrose; and 5-150 mM amino acid component selected from the group consisting of proline, glycine, lysine, and any combination thereof.
N391. 1-150 mg/mL EGFR/LGR5 BsAb; 3-25 mM phosphate buffer system providing a composition pH of 5.8-8.1; 150-350 mM sucrose; and 5-150 mM amino acid component selected from the group consisting of proline, glycine, lysine, and any combination thereof.
N392. 10-60 mg/mL EGFR/LGR5 BsAb; 3-25 mM phosphate buffer system providing a composition pH of 5.8-8.1; 150-350 mM sucrose; and 5-150 mM amino acid component selected from the group consisting of proline, glycine, lysine, and any combination thereof.
N393. 1-150 mg/mL EGFR/LGR5 BsAb; 3-25 mM citrate-phosphate buffer system providing a composition pH of 4-7.1; 150-350 mM sucrose; and 5-150 mM amino acid component selected from the group consisting of proline, glycine, lysine, and any combination thereof.
N394. 10-60 mg/mL EGFR/LGR5 BsAb; 3-25 mM citrate-phosphate buffer system providing a composition pH of 4-7.1; 150-350 mM sucrose; and 5-150 mM amino acid component selected from the group consisting of proline, glycine, lysine, and any combination thereof.
N395. 1-150 mg/mL EGFR/LGR5 BsAb; 3-25 mM histidine buffer system providing a composition pH of 5.4-7.1; 150-350 mM maltose; and 5-150 mM amino acid component selected from the group consisting of proline, glycine, lysine, and any combination thereof.
N396. 10-60 mg/mL EGFR/LGR5 BsAb; 3-25 mM histidine buffer system providing a composition pH of 5.4-7.1; 150-350 mM maltose; and 5-150 mM amino acid component selected from the group consisting of proline, glycine, lysine, and any combination thereof.
N397. 1-150 mg/mL EGFR/LGR5 BsAb; 3-25 mM acetate buffer system providing a composition pH of 4-5.6; 150-350 mM maltose; and 5-150 mM amino acid component selected from the group consisting of proline, glycine, lysine, and any combination thereof.
N398. 10-60 mg/mL EGFR/LGR5 BsAb; 3-25 mM acetate buffer system providing a composition pH of 4-5.6; 150-350 mM maltose; and 5-150 mM amino acid component selected from the group consisting of proline, glycine, lysine, and any combination thereof.
N399. 1-150 mg/mL EGFR/LGR5 BsAb; 3-25 mM citrate buffer system providing a composition pH of 4.5-7.5; 150-350 mM maltose; and 5-150 mM amino acid component selected from the group consisting of proline, glycine, lysine, and any combination thereof.
N400. 10-60 mg/mL EGFR/LGR5 BsAb; 3-25 mM citrate buffer system providing a composition pH of 4.5-7.5; 150-350 mM maltose; and 5-150 mM amino acid component selected from the group consisting of proline, glycine, lysine, and any combination thereof.
N401. 1-150 mg/mL EGFR/LGR5 BsAb; 3-25 mM succinate buffer system providing a composition pH of 4-6.6; 150-350 mM maltose; and 5-150 mM amino acid component selected from the group consisting of proline, glycine, lysine, and any combination thereof.
N402. 10-60 mg/mL EGFR/LGR5 BsAb; 3-25 mM succinate buffer system providing a composition pH of 4-6.6; 150-350 mM maltose; and 5-150 mM amino acid component selected from the group consisting of proline, glycine, lysine, and any combination thereof.
N403. 1-150 mg/mL EGFR/LGR5 BsAb; 3-25 mM phosphate buffer system providing a composition pH of 5.8-8.1; 150-350 mM maltose; and 5-150 mM amino acid component selected from the group consisting of proline, glycine, lysine, and any combination thereof.
N404. 10-60 mg/mL EGFR/LGR5 BsAb; 3-25 mM phosphate buffer system providing a composition pH of 5.8-8.1; 150-350 mM maltose; and 5-150 mM amino acid component selected from the group consisting of proline, glycine, lysine, and any combination thereof.
N405. 1-150 mg/mL EGFR/LGR5 BsAb; 3-25 mM citrate-phosphate buffer system providing a composition pH of 4-7.1; 150-350 mM maltose; and 5-150 mM amino acid component selected from the group consisting of proline, glycine, lysine, and any combination thereof.
N406. 10-60 mg/mL EGFR/LGR5 BsAb; 3-25 mM citrate-phosphate buffer system providing a composition pH of 4-7.1; 150-350 mM maltose; and 5-150 mM amino acid component selected from the group consisting of proline, glycine, lysine, and any combination thereof.
N407. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 2-25 mM methionine.
N408. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 2-25 mM methionine.
N409. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 2-25 mM methionine.
N410. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 2-25 mM methionine.
N411. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 2-25 mM methionine.
N412. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 2-25 mM methionine.
N413. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 2-25 mM methionine.
N414. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 2-25 mM methionine.
N415. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 2-25 mM methionine.
N416. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 2-25 mM methionine.
N417. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 2-25 mM methionine.
N418. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 2-25 mM methionine.
N419. 1-150 mg/mL EGFR/LGR5 BsAb; 3-25 mM histidine buffer system providing a composition pH of 5.4-7.1; 150-350 mM sucrose; and 2-25 mM methionine.
N420. 10-60 mg/mL EGFR/LGR5 BsAb; 3-25 mM histidine buffer system providing a composition pH of 5.4-7.1; 150-350 mM sucrose; and 2-25 mM methionine.
N421. 1-150 mg/mL EGFR/LGR5 BsAb; 3-25 mM acetate buffer system providing a composition pH of 4-5.6; 150-350 mM sucrose; and 2-25 mM methionine.
N422. 10-60 mg/mL EGFR/LGR5 BsAb; 3-25 mM acetate buffer system providing a composition pH of 4-5.6; 150-350 mM sucrose; and 2-25 mM methionine.
N423. 1-150 mg/mL EGFR/LGR5 BsAb; 3-25 mM citrate buffer system providing a composition pH of 4.5-7.5; 150-350 mM sucrose; and 2-25 mM methionine.
N424. 10-60 mg/mL EGFR/LGR5 BsAb; 3-25 mM citrate buffer system providing a composition pH of 4.5-7.5; 150-350 mM sucrose; and 2-25 mM methionine.
N425. 1-150 mg/mL EGFR/LGR5 BsAb; 3-25 mM succinate buffer system providing a composition pH of 4-6.6; 150-350 mM sucrose; and 2-25 mM methionine.
N426. 10-60 mg/mL EGFR/LGR5 BsAb; 3-25 mM succinate buffer system providing a composition pH of 4-6.6; 150-350 mM sucrose; and 2-25 mM methionine.
N427. 1-150 mg/mL EGFR/LGR5 BsAb; 3-25 mM phosphate buffer system providing a composition pH of 5.8-8.1; 150-350 mM sucrose; and 2-25 mM methionine.
N428. 10-60 mg/mL EGFR/LGR5 BsAb; 3-25 mM phosphate buffer system providing a composition pH of 5.8-8.1; 150-350 mM sucrose; and 2-25 mM methionine.
N429. 1-150 mg/mL EGFR/LGR5 BsAb; 3-25 mM citrate-phosphate buffer system providing a composition pH of 4-7.1; 150-350 mM sucrose; and 2-25 mM methionine.
N430. 10-60 mg/mL EGFR/LGR5 BsAb; 3-25 mM citrate-phosphate buffer system providing a composition pH of 4-7.1; 150-350 mM sucrose; and 2-25 mM methionine.
N431. 1-150 mg/mL EGFR/LGR5 BsAb; 3-25 mM histidine buffer system providing a composition pH of 5.4-7.1; 150-350 mM maltose; and 2-25 mM methionine.
N432. 10-60 mg/mL EGFR/LGR5 BsAb; 3-25 mM histidine buffer system providing a composition pH of 5.4-7.1; 150-350 mM maltose; and 2-25 mM methionine.
N433. 1-150 mg/mL EGFR/LGR5 BsAb; 3-25 mM acetate buffer system providing a composition pH of 4-5.6; 150-350 mM maltose; and 2-25 mM methionine.
N434. 10-60 mg/mL EGFR/LGR5 BsAb; 3-25 mM acetate buffer system providing a composition pH of 4-5.6; 150-350 mM maltose; and 2-25 mM methionine.
N435. 1-150 mg/mL EGFR/LGR5 BsAb; 3-25 mM citrate buffer system providing a composition pH of 4.5-7.5; 150-350 mM maltose; and 2-25 mM methionine.
N436. 10-60 mg/mL EGFR/LGR5 BsAb; 3-25 mM citrate buffer system providing a composition pH of 4.5-7.5; 150-350 mM maltose; and 2-25 mM methionine.
N437. 1-150 mg/mL EGFR/LGR5 BsAb; 3-25 mM succinate buffer system providing a composition pH of 4-6.6; 150-350 mM maltose; and 2-25 mM methionine.
N438. 10-60 mg/mL EGFR/LGR5 BsAb; 3-25 mM succinate buffer system providing a composition pH of 4-6.6; 150-350 mM maltose; and 2-25 mM methionine.
N439. 1-150 mg/mL EGFR/LGR5 BsAb; 3-25 mM phosphate buffer system providing a composition pH of 5.8-8.1; 150-350 mM maltose; and 2-25 mM methionine.
N440. 10-60 mg/mL EGFR/LGR5 BsAb; 3-25 mM phosphate buffer system providing a composition pH of 5.8-8.1; 150-350 mM maltose; and 2-25 mM methionine.
N441. 1-150 mg/mL EGFR/LGR5 BsAb; 3-25 mM citrate-phosphate buffer system providing a composition pH of 4-7.1; 150-350 mM maltose; and 2-25 mM methionine.
N442. 10-60 mg/mL EGFR/LGR5 BsAb; 3-25 mM citrate-phosphate buffer system providing a composition pH of 4-7.1; 150-350 mM maltose; and 2-25 mM methionine.
N443. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.01-0.2 mM EDTA.
N444. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; and 0.01-0.2 mM EDTA.
N445. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.01-0.2 mM EDTA.
N446. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; and 0.01-0.2 mM EDTA.
N447. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.01-0.2 mM EDTA.
N448. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; and 0.01-0.2 mM EDTA.
N449. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.01-0.2 mM EDTA.
N450. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; and 0.01-0.2 mM EDTA.
N451. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.01-0.2 mM EDTA.
N452. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; and 0.01-0.2 mM EDTA.
N453. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.01-0.2 mM EDTA.
N454. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; and 0.01-0.2 mM EDTA.
N455. 1-150 mg/mL EGFR/LGR5 BsAb; 3-25 mM histidine buffer system providing a composition pH of 5.4-7.1; 150-350 mM sucrose; and 0.01-0.2 mM EDTA.
N456. 10-60 mg/mL EGFR/LGR5 BsAb; 3-25 mM histidine buffer system providing a composition pH of 5.4-7.1; 150-350 mM sucrose; and 0.01-0.2 mM EDTA.
N457. 1-150 mg/mL EGFR/LGR5 BsAb; 3-25 mM acetate buffer system providing a composition pH of 4-5.6; 150-350 mM sucrose; and 0.01-0.2 mM EDTA.
N458. 10-60 mg/mL EGFR/LGR5 BsAb; 3-25 mM acetate buffer system providing a composition pH of 4-5.6; 150-350 mM sucrose; and 0.01-0.2 mM EDTA.
N459. 1-150 mg/mL EGFR/LGR5 BsAb; 3-25 mM citrate buffer system providing a composition pH of 4.5-7.5; 150-350 mM sucrose; and 0.01-0.2 mM EDTA.
N460. 10-60 mg/mL EGFR/LGR5 BsAb; 3-25 mM citrate buffer system providing a composition pH of 4.5-7.5; 150-350 mM sucrose; and 0.01-0.2 mM EDTA.
N461. 1-150 mg/mL EGFR/LGR5 BsAb; 3-25 mM succinate buffer system providing a composition pH of 4-6.6; 150-350 mM sucrose; and 0.01-0.2 mM EDTA.
N462. 10-60 mg/mL EGFR/LGR5 BsAb; 3-25 mM succinate buffer system providing a composition pH of 4-6.6; 150-350 mM sucrose; and 0.01-0.2 mM EDTA.
N463. 1-150 mg/mL EGFR/LGR5 BsAb; 3-25 mM phosphate buffer system providing a composition pH of 5.8-8.1; 150-350 mM sucrose; and 0.01-0.2 mM EDTA.
N464. 10-60 mg/mL EGFR/LGR5 BsAb; 3-25 mM phosphate buffer system providing a composition pH of 5.8-8.1; 150-350 mM sucrose; and 0.01-0.2 mM EDTA.
N465. 1-150 mg/mL EGFR/LGR5 BsAb; 3-25 mM citrate-phosphate buffer system providing a composition pH of 4-7.1; 150-350 mM sucrose; and 0.01-0.2 mM EDTA.
N466. 10-60 mg/mL EGFR/LGR5 BsAb; 3-25 mM citrate-phosphate buffer system providing a composition pH of 4-7.1; 150-350 mM sucrose; and 0.01-0.2 mM EDTA.
N467. 1-150 mg/mL EGFR/LGR5 BsAb; 3-25 mM histidine buffer system providing a composition pH of 5.4-7.1; 150-350 mM maltose; and 0.01-0.2 mM EDTA.
N468. 10-60 mg/mL EGFR/LGR5 BsAb; 3-25 mM histidine buffer system providing a composition pH of 5.4-7.1; 150-350 mM maltose; and 0.01-0.2 mM EDTA.
N469. 1-150 mg/mL EGFR/LGR5 BsAb; 3-25 mM acetate buffer system providing a composition pH of 4-5.6; 150-350 mM maltose; and 0.01-0.2 mM EDTA.
N470. 10-60 mg/mL EGFR/LGR5 BsAb; 3-25 mM acetate buffer system providing a composition pH of 4-5.6; 150-350 mM maltose; and 0.01-0.2 mM EDTA.
N471. 1-150 mg/mL EGFR/LGR5 BsAb; 3-25 mM citrate buffer system providing a composition pH of 4.5-7.5; 150-350 mM maltose; and 0.01-0.2 mM EDTA.
N472. 10-60 mg/mL EGFR/LGR5 BsAb; 3-25 mM citrate buffer system providing a composition pH of 4.5-7.5; 150-350 mM maltose; and 0.01-0.2 mM EDTA.
N473. 1-150 mg/mL EGFR/LGR5 BsAb; 3-25 mM succinate buffer system providing a composition pH of 4-6.6; 150-350 mM maltose; and 0.01-0.2 mM EDTA.
N474. 10-60 mg/mL EGFR/LGR5 BsAb; 3-25 mM succinate buffer system providing a composition pH of 4-6.6; 150-350 mM maltose; and 0.01-0.2 mM EDTA.
N475. 1-150 mg/mL EGFR/LGR5 BsAb; 3-25 mM phosphate buffer system providing a composition pH of 5.8-8.1; 150-350 mM maltose; and 0.01-0.2 mM EDTA.
N476. 10-60 mg/mL EGFR/LGR5 BsAb; 3-25 mM phosphate buffer system providing a composition pH of 5.8-8.1; 150-350 mM maltose; and 0.01-0.2 mM EDTA.
N477. 1-150 mg/mL EGFR/LGR5 BsAb; 3-25 mM citrate-phosphate buffer system providing a composition pH of 4-7.1; 150-350 mM maltose; and 0.01-0.2 mM EDTA.
N478. 10-60 mg/mL EGFR/LGR5 BsAb; 3-25 mM citrate-phosphate buffer system providing a composition pH of 4-7.1; 150-350 mM maltose; and 0.01-0.2 mM EDTA.
N479. 1-150 mg/mL EGFR/LGR5 BsAb; 3-25 mM histidine buffer system providing a composition pH of 5.4-7.1; 0.4-1.2 mg/mL surfactant; and 5-150 mM amino acid component selected from the group consisting of proline, glycine, lysine, and any combination thereof.
N480. 10-60 mg/mL EGFR/LGR5 BsAb; 3-25 mM histidine buffer system providing a composition pH of 5.4-7.1; 0.4-1.2 mg/mL surfactant; and 5-150 mM amino acid component selected from the group consisting of proline, glycine, lysine, and any combination thereof.
N481. 1-150 mg/mL EGFR/LGR5 BsAb; 3-25 mM acetate buffer system providing a composition pH of 4-5.6; 0.4-1.2 mg/mL surfactant; and 5-150 mM amino acid component selected from the group consisting of proline, glycine, lysine, and any combination thereof.
N482. 10-60 mg/mL EGFR/LGR5 BsAb; 3-25 mM acetate buffer system providing a composition pH of 4-5.6; 0.4-1.2 mg/mL surfactant; and 5-150 mM amino acid component selected from the group consisting of proline, glycine, lysine, and any combination thereof.
N483. 1-150 mg/mL EGFR/LGR5 BsAb; 3-25 mM citrate buffer system providing a composition pH of 4.5-7.5; 0.4-1.2 mg/mL surfactant; and 5-150 mM amino acid component selected from the group consisting of proline, glycine, lysine, and any combination thereof.
N484. 10-60 mg/mL EGFR/LGR5 BsAb; 3-25 mM citrate buffer system providing a composition pH of 4.5-7.5; 0.4-1.2 mg/mL surfactant; and 5-150 mM amino acid component selected from the group consisting of proline, glycine, lysine, and any combination thereof.
N485. 1-150 mg/mL EGFR/LGR5 BsAb; 3-25 mM succinate buffer system providing a composition pH of 4-6.6; 0.4-1.2 mg/mL surfactant; and 5-150 mM amino acid component selected from the group consisting of proline, glycine, lysine, and any combination thereof.
N486. 10-60 mg/mL EGFR/LGR5 BsAb; 3-25 mM succinate buffer system providing a composition pH of 4-6.6; 0.4-1.2 mg/mL surfactant; and 5-150 mM amino acid component selected from the group consisting of proline, glycine, lysine, and any combination thereof.
N487. 1-150 mg/mL EGFR/LGR5 BsAb; 3-25 mM phosphate buffer system providing a composition pH of 5.8-8.1; 0.4-1.2 mg/mL surfactant; and 5-150 mM amino acid component selected from the group consisting of proline, glycine, lysine, and any combination thereof.
N488. 10-60 mg/mL EGFR/LGR5 BsAb; 3-25 mM phosphate buffer system providing a composition pH of 5.8-8.1; 0.4-1.2 mg/mL surfactant; and 5-150 mM amino acid component selected from the group consisting of proline, glycine, lysine, and any combination thereof.
N489. 1-150 mg/mL EGFR/LGR5 BsAb; 3-25 mM citrate-phosphate buffer system providing a composition pH of 4-7.1; 0.4-1.2 mg/mL surfactant; and 5-150 mM amino acid component selected from the group consisting of proline, glycine, lysine, and any combination thereof.
N490. 10-60 mg/mL EGFR/LGR5 BsAb; 3-25 mM citrate-phosphate buffer system providing a composition pH of 4-7.1; 0.4-1.2 mg/mL surfactant; and 5-150 mM amino acid component selected from the group consisting of proline, glycine, lysine, and any combination thereof.
N491. 1-150 mg/mL EGFR/LGR5 BsAb; 3-25 mM histidine buffer system providing a composition pH of 5.4-7.1; 0.1-1.1 mg/mL polysorbate 80; and 5-150 mM amino acid component selected from the group consisting of proline, glycine, lysine, and any combination thereof.
N492. 10-60 mg/mL EGFR/LGR5 BsAb; 3-25 mM histidine buffer system providing a composition pH of 5.4-7.1; 0.1-1.1 mg/mL polysorbate 80; and 5-150 mM amino acid component selected from the group consisting of proline, glycine, lysine, and any combination thereof.
N493. 1-150 mg/mL EGFR/LGR5 BsAb; 3-25 mM acetate buffer system providing a composition pH of 4-5.6; 0.1-1.1 mg/mL polysorbate 80; and 5-150 mM amino acid component selected from the group consisting of proline, glycine, lysine, and any combination thereof.
N494. 10-60 mg/mL EGFR/LGR5 BsAb; 3-25 mM acetate buffer system providing a composition pH of 4-5.6; 0.1-1.1 mg/mL polysorbate 80; and 5-150 mM amino acid component selected from the group consisting of proline, glycine, lysine, and any combination thereof.
N495. 1-150 mg/mL EGFR/LGR5 BsAb; 3-25 mM citrate buffer system providing a composition pH of 4.5-7.5; 0.1-1.1 mg/mL polysorbate 80; and 5-150 mM amino acid component selected from the group consisting of proline, glycine, lysine, and any combination thereof.
N496. 10-60 mg/mL EGFR/LGR5 BsAb; 3-25 mM citrate buffer system providing a composition pH of 4.5-7.5; 0.1-1.1 mg/mL polysorbate 80; and 5-150 mM amino acid component selected from the group consisting of proline, glycine, lysine, and any combination thereof.
N497. 1-150 mg/mL EGFR/LGR5 BsAb; 3-25 mM succinate buffer system providing a composition pH of 4-6.6; 0.1-1.1 mg/mL polysorbate 80; and 5-150 mM amino acid component selected from the group consisting of proline, glycine, lysine, and any combination thereof.
N498. 10-60 mg/mL EGFR/LGR5 BsAb; 3-25 mM succinate buffer system providing a composition pH of 4-6.6; 0.1-1.1 mg/mL polysorbate 80; and 5-150 mM amino acid component selected from the group consisting of proline, glycine, lysine, and any combination thereof.
N499. 1-150 mg/mL EGFR/LGR5 BsAb; 3-25 mM phosphate buffer system providing a composition pH of 5.8-8.1; 0.1-1.1 mg/mL polysorbate 80; and 5-150 mM amino acid component selected from the group consisting of proline, glycine, lysine, and any combination thereof.
N500. 10-60 mg/mL EGFR/LGR5 BsAb; 3-25 mM phosphate buffer system providing a composition pH of 5.8-8.1; 0.1-1.1 mg/mL polysorbate 80; and 5-150 mM amino acid component selected from the group consisting of proline, glycine, lysine, and any combination thereof.
N501. 1-150 mg/mL EGFR/LGR5 BsAb; 3-25 mM citrate-phosphate buffer system providing a composition pH of 4-7.1; 0.1-1.1 mg/mL polysorbate 80; and 5-150 mM amino acid component selected from the group consisting of proline, glycine, lysine, and any combination thereof.
N502. 10-60 mg/mL EGFR/LGR5 BsAb; 3-25 mM citrate-phosphate buffer system providing a composition pH of 4-7.1; 0.1-1.1 mg/mL polysorbate 80; and 5-150 mM amino acid component selected from the group consisting of proline, glycine, lysine, and any combination thereof.
N503. 1-150 mg/mL EGFR/LGR5 BsAb; 3-25 mM histidine buffer system providing a composition pH of 5.4-7.1; 0.4-1.2 mg/mL surfactant; and 2-25 mM methionine.
N504. 10-60 mg/mL EGFR/LGR5 BsAb; 3-25 mM histidine buffer system providing a composition pH of 5.4-7.1; 0.4-1.2 mg/mL surfactant; and 2-25 mM methionine.
N505. 1-150 mg/mL EGFR/LGR5 BsAb; 3-25 mM acetate buffer system providing a composition pH of 4-5.6; 0.4-1.2 mg/mL surfactant; and 2-25 mM methionine.
N506. 10-60 mg/mL EGFR/LGR5 BsAb; 3-25 mM acetate buffer system providing a composition pH of 4-5.6; 0.4-1.2 mg/mL surfactant; and 2-25 mM methionine.
N507. 1-150 mg/mL EGFR/LGR5 BsAb; 3-25 mM citrate buffer system providing a composition pH of 4.5-7.5; 0.4-1.2 mg/mL surfactant; and 2-25 mM methionine.
N508. 10-60 mg/mL EGFR/LGR5 BsAb; 3-25 mM citrate buffer system providing a composition pH of 4.5-7.5; 0.4-1.2 mg/mL surfactant; and 2-25 mM methionine.
N509. 1-150 mg/mL EGFR/LGR5 BsAb; 3-25 mM succinate buffer system providing a composition pH of 4-6.6; 0.4-1.2 mg/mL surfactant; and 2-25 mM methionine.
N510. 10-60 mg/mL EGFR/LGR5 BsAb; 3-25 mM succinate buffer system providing a composition pH of 4-6.6; 0.4-1.2 mg/mL surfactant; and 2-25 mM methionine.
N511. 1-150 mg/mL EGFR/LGR5 BsAb; 3-25 mM phosphate buffer system providing a composition pH of 5.8-8.1; 0.4-1.2 mg/mL surfactant; and 2-25 mM methionine.
N512. 10-60 mg/mL EGFR/LGR5 BsAb; 3-25 mM phosphate buffer system providing a composition pH of 5.8-8.1; 0.4-1.2 mg/mL surfactant; and 2-25 mM methionine.
N513. 1-150 mg/mL EGFR/LGR5 BsAb; 3-25 mM citrate-phosphate buffer system providing a composition pH of 4-7.1; 0.4-1.2 mg/mL surfactant; and 2-25 mM methionine.
N514. 10-60 mg/mL EGFR/LGR5 BsAb; 3-25 mM citrate-phosphate buffer system providing a composition pH of 4-7.1; 0.4-1.2 mg/mL surfactant; and 2-25 mM methionine.
N515. 1-150 mg/mL EGFR/LGR5 BsAb; 3-25 mM histidine buffer system providing a composition pH of 5.4-7.1; 0.1-1.1 mg/mL polysorbate 80; and 2-25 mM methionine.
N516. 10-60 mg/mL EGFR/LGR5 BsAb; 3-25 mM histidine buffer system providing a composition pH of 5.4-7.1; 0.1-1.1 mg/mL polysorbate 80; and 2-25 mM methionine.
N517. 1-150 mg/mL EGFR/LGR5 BsAb; 3-25 mM acetate buffer system providing a composition pH of 4-5.6; 0.1-1.1 mg/mL polysorbate 80; and 2-25 mM methionine.
N518. 10-60 mg/mL EGFR/LGR5 BsAb; 3-25 mM acetate buffer system providing a composition pH of 4-5.6; 0.1-1.1 mg/mL polysorbate 80; and 2-25 mM methionine.
N519. 1-150 mg/mL EGFR/LGR5 BsAb; 3-25 mM citrate buffer system providing a composition pH of 4.5-7.5; 0.1-1.1 mg/mL polysorbate 80; and 2-25 mM methionine.
N520. 10-60 mg/mL EGFR/LGR5 BsAb; 3-25 mM citrate buffer system providing a composition pH of 4.5-7.5; 0.1-1.1 mg/mL polysorbate 80; and 2-25 mM methionine.
N521. 1-150 mg/mL EGFR/LGR5 BsAb; 3-25 mM succinate buffer system providing a composition pH of 4-6.6; 0.1-1.1 mg/mL polysorbate 80; and 2-25 mM methionine.
N522. 10-60 mg/mL EGFR/LGR5 BsAb; 3-25 mM succinate buffer system providing a composition pH of 4-6.6; 0.1-1.1 mg/mL polysorbate 80; and 2-25 mM methionine.
N523. 1-150 mg/mL EGFR/LGR5 BsAb; 3-25 mM phosphate buffer system providing a composition pH of 5.8-8.1; 0.1-1.1 mg/mL polysorbate 80; and 2-25 mM methionine.
N524. 10-60 mg/mL EGFR/LGR5 BsAb; 3-25 mM phosphate buffer system providing a composition pH of 5.8-8.1; 0.1-1.1 mg/mL polysorbate 80; and 2-25 mM methionine.
N525. 1-150 mg/mL EGFR/LGR5 BsAb; 3-25 mM citrate-phosphate buffer system providing a composition pH of 4-7.1; 0.1-1.1 mg/mL polysorbate 80; and 2-25 mM methionine.
N526. 10-60 mg/mL EGFR/LGR5 BsAb; 3-25 mM citrate-phosphate buffer system providing a composition pH of 4-7.1; 0.1-1.1 mg/mL polysorbate 80; and 2-25 mM methionine.
N527. 1-150 mg/mL EGFR/LGR5 BsAb; 3-25 mM histidine buffer system providing a composition pH of 5.4-7.1; 0.4-1.2 mg/mL surfactant; and 0.01-0.2 mM EDTA.
N528. 10-60 mg/mL EGFR/LGR5 BsAb; 3-25 mM histidine buffer system providing a composition pH of 5.4-7.1; 0.4-1.2 mg/mL surfactant; and 0.01-0.2 mM EDTA.
N529. 1-150 mg/mL EGFR/LGR5 BsAb; 3-25 mM acetate buffer system providing a composition pH of 4-5.6; 0.4-1.2 mg/mL surfactant; and 0.01-0.2 mM EDTA.
N530. 10-60 mg/mL EGFR/LGR5 BsAb; 3-25 mM acetate buffer system providing a composition pH of 4-5.6; 0.4-1.2 mg/mL surfactant; and 0.01-0.2 mM EDTA.
N531. 1-150 mg/mL EGFR/LGR5 BsAb; 3-25 mM citrate buffer system providing a composition pH of 4.5-7.5; 0.4-1.2 mg/mL surfactant; and 0.01-0.2 mM EDTA.
N532. 10-60 mg/mL EGFR/LGR5 BsAb; 3-25 mM citrate buffer system providing a composition pH of 4.5-7.5; 0.4-1.2 mg/mL surfactant; and 0.01-0.2 mM EDTA.
N533. 1-150 mg/mL EGFR/LGR5 BsAb; 3-25 mM succinate buffer system providing a composition pH of 4-6.6; 0.4-1.2 mg/mL surfactant; and 0.01-0.2 mM EDTA.
N534. 10-60 mg/mL EGFR/LGR5 BsAb; 3-25 mM succinate buffer system providing a composition pH of 4-6.6; 0.4-1.2 mg/mL surfactant; and 0.01-0.2 mM EDTA.
N535. 1-150 mg/mL EGFR/LGR5 BsAb; 3-25 mM phosphate buffer system providing a composition pH of 5.8-8.1; 0.4-1.2 mg/mL surfactant; and 0.01-0.2 mM EDTA.
N536. 10-60 mg/mL EGFR/LGR5 BsAb; 3-25 mM phosphate buffer system providing a composition pH of 5.8-8.1; 0.4-1.2 mg/mL surfactant; and 0.01-0.2 mM EDTA.
N537. 1-150 mg/mL EGFR/LGR5 BsAb; 3-25 mM citrate-phosphate buffer system providing a composition pH of 4-7.1; 0.4-1.2 mg/mL surfactant; and 0.01-0.2 mM EDTA.
N538. 10-60 mg/mL EGFR/LGR5 BsAb; 3-25 mM citrate-phosphate buffer system providing a composition pH of 4-7.1; 0.4-1.2 mg/mL surfactant; and 0.01-0.2 mM EDTA.
N539. 1-150 mg/mL EGFR/LGR5 BsAb; 3-25 mM histidine buffer system providing a composition pH of 5.4-7.1; 0.1-1.1 mg/mL polysorbate 80; and 0.01-0.2 mM EDTA.
N540. 10-60 mg/mL EGFR/LGR5 BsAb; 3-25 mM histidine buffer system providing a composition pH of 5.4-7.1; 0.1-1.1 mg/mL polysorbate 80; and 0.01-0.2 mM EDTA.
N541. 1-150 mg/mL EGFR/LGR5 BsAb; 3-25 mM acetate buffer system providing a composition pH of 4-5.6; 0.1-1.1 mg/mL polysorbate 80; and 0.01-0.2 mM EDTA.
N542. 10-60 mg/mL EGFR/LGR5 BsAb; 3-25 mM acetate buffer system providing a composition pH of 4-5.6; 0.1-1.1 mg/mL polysorbate 80; and 0.01-0.2 mM EDTA.
N543. 1-150 mg/mL EGFR/LGR5 BsAb; 3-25 mM citrate buffer system providing a composition pH of 4.5-7.5; 0.1-1.1 mg/mL polysorbate 80; and 0.01-0.2 mM EDTA.
N544. 10-60 mg/mL EGFR/LGR5 BsAb; 3-25 mM citrate buffer system providing a composition pH of 4.5-7.5; 0.1-1.1 mg/mL polysorbate 80; and 0.01-0.2 mM EDTA.
N545. 1-150 mg/mL EGFR/LGR5 BsAb; 3-25 mM succinate buffer system providing a composition pH of 4-6.6; 0.1-1.1 mg/mL polysorbate 80; and 0.01-0.2 mM EDTA.
N546. 10-60 mg/mL EGFR/LGR5 BsAb; 3-25 mM succinate buffer system providing a composition pH of 4-6.6; 0.1-1.1 mg/mL polysorbate 80; and 0.01-0.2 mM EDTA.
N547. 1-150 mg/mL EGFR/LGR5 BsAb; 3-25 mM phosphate buffer system providing a composition pH of 5.8-8.1; 0.1-1.1 mg/mL polysorbate 80; and 0.01-0.2 mM EDTA.
N548. 10-60 mg/mL EGFR/LGR5 BsAb; 3-25 mM phosphate buffer system providing a composition pH of 5.8-8.1; 0.1-1.1 mg/mL polysorbate 80; and 0.01-0.2 mM EDTA.
N549. 1-150 mg/mL EGFR/LGR5 BsAb; 3-25 mM citrate-phosphate buffer system providing a composition pH of 4-7.1; 0.1-1.1 mg/mL polysorbate 80; and 0.01-0.2 mM EDTA.
N550. 10-60 mg/mL EGFR/LGR5 BsAb; 3-25 mM citrate-phosphate buffer system providing a composition pH of 4-7.1; 0.1-1.1 mg/mL polysorbate 80; and 0.01-0.2 mM EDTA.
N551. 1-150 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; and 2-25 mM methionine.
N552. 10-60 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; and 2-25 mM methionine.
N553. 1-150 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; and 2-25 mM methionine.
N554. 10-60 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; and 2-25 mM methionine.
N555. 1-150 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; and 2-25 mM methionine.
N556. 10-60 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; and 2-25 mM methionine.
N557. 1-150 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; and 2-25 mM methionine.
N558. 10-60 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; and 2-25 mM methionine.
N559. 1-150 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; and 2-25 mM methionine.
N560. 10-60 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; and 2-25 mM methionine.
N561. 1-150 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; and 2-25 mM methionine.
N562. 10-60 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; and 2-25 mM methionine.
N563. 1-150 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; and 0.01-0.2 mM EDTA.
N564. 10-60 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; and 0.01-0.2 mM EDTA.
N565. 1-150 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; and 0.01-0.2 mM EDTA.
N566. 10-60 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; and 0.01-0.2 mM EDTA.
N567. 1-150 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; and 0.01-0.2 mM EDTA.
N568. 10-60 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; and 0.01-0.2 mM EDTA.
N569. 1-150 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; and 0.01-0.2 mM EDTA.
N570. 10-60 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; and 0.01-0.2 mM EDTA.
N571. 1-150 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; and 0.01-0.2 mM EDTA.
N572. 10-60 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; and 0.01-0.2 mM EDTA.
N573. 1-150 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; and 0.01-0.2 mM EDTA.
N574. 10-60 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; and 0.01-0.2 mM EDTA.
N575. 1-150 mg/mL EGFR/LGR5 BsAb; 3-25 mM histidine buffer system providing a composition pH of 5.4-7.1; 2-25 mM methionine; and 0.01-0.2 mM EDTA.
N576. 10-60 mg/mL EGFR/LGR5 BsAb; 3-25 mM histidine buffer system providing a composition pH of 5.4-7.1; 2-25 mM methionine; and 0.01-0.2 mM EDTA.
N577. 1-150 mg/mL EGFR/LGR5 BsAb; 3-25 mM acetate buffer system providing a composition pH of 4-5.6; 2-25 mM methionine; and 0.01-0.2 mM EDTA.
N578. 10-60 mg/mL EGFR/LGR5 BsAb; 3-25 mM acetate buffer system providing a composition pH of 4-5.6; 2-25 mM methionine; and 0.01-0.2 mM EDTA.
N579. 1-150 mg/mL EGFR/LGR5 BsAb; 3-25 mM citrate buffer system providing a composition pH of 4.5-7.5; 2-25 mM methionine; and 0.01-0.2 mM EDTA.
N580. 10-60 mg/mL EGFR/LGR5 BsAb; 3-25 mM citrate buffer system providing a composition pH of 4.5-7.5; 2-25 mM methionine; and 0.01-0.2 mM EDTA.
N581. 1-150 mg/mL EGFR/LGR5 BsAb; 3-25 mM succinate buffer system providing a composition pH of 4-6.6; 2-25 mM methionine; and 0.01-0.2 mM EDTA.
N582. 10-60 mg/mL EGFR/LGR5 BsAb; 3-25 mM succinate buffer system providing a composition pH of 4-6.6; 2-25 mM methionine; and 0.01-0.2 mM EDTA.
N583. 1-150 mg/mL EGFR/LGR5 BsAb; 3-25 mM phosphate buffer system providing a composition pH of 5.8-8.1; 2-25 mM methionine; and 0.01-0.2 mM EDTA.
N584. 10-60 mg/mL EGFR/LGR5 BsAb; 3-25 mM phosphate buffer system providing a composition pH of 5.8-8.1; 2-25 mM methionine; and 0.01-0.2 mM EDTA.
N585. 1-150 mg/mL EGFR/LGR5 BsAb; 3-25 mM citrate-phosphate buffer system providing a composition pH of 4-7.1; 2-25 mM methionine; and 0.01-0.2 mM EDTA.
N586. 10-60 mg/mL EGFR/LGR5 BsAb; 3-25 mM citrate-phosphate buffer system providing a composition pH of 4-7.1; 2-25 mM methionine; and 0.01-0.2 mM EDTA.
N587. 1-150 mg/mL EGFR/LGR5 BsAb; 10-180 mM sodium chloride; 100-400 mM sugar component selected from the group consisting of sucrose, mannitol, sorbitol, and maltose; and 0.4-1.2 mg/mL surfactant.
N588. 10-60 mg/mL EGFR/LGR5 BsAb; 10-180 mM sodium chloride; 100-400 mM sugar component selected from the group consisting of sucrose, mannitol, sorbitol, and maltose; and 0.4-1.2 mg/mL surfactant.
N589. 1-150 mg/mL EGFR/LGR5 BsAb; 10-180 mM sodium chloride; 150-350 mM sucrose; and 0.4-1.2 mg/mL surfactant.
N590. 10-60 mg/mL EGFR/LGR5 BsAb; 10-180 mM sodium chloride; 150-350 mM sucrose; and 0.4-1.2 mg/mL surfactant.
N591. 1-150 mg/mL EGFR/LGR5 BsAb; 10-180 mM sodium chloride; 150-350 mM maltose; and 0.4-1.2 mg/mL surfactant.
N592. 10-60 mg/mL EGFR/LGR5 BsAb; 10-180 mM sodium chloride; 150-350 mM maltose; and 0.4-1.2 mg/mL surfactant.
N593. 1-150 mg/mL EGFR/LGR5 BsAb; 10-180 mM sodium chloride; 100-400 mM sugar component selected from the group consisting of sucrose, mannitol, sorbitol, and maltose; and 0.1-1.1 mg/mL polysorbate 80.
N594. 10-60 mg/mL EGFR/LGR5 BsAb; 10-180 mM sodium chloride; 100-400 mM sugar component selected from the group consisting of sucrose, mannitol, sorbitol, and maltose; and 0.1-1.1 mg/mL polysorbate 80.
N595. 1-150 mg/mL EGFR/LGR5 BsAb; 10-180 mM sodium chloride; 150-350 mM sucrose; and 0.1-1.1 mg/mL polysorbate 80.
N596. 10-60 mg/mL EGFR/LGR5 BsAb; 10-180 mM sodium chloride; 150-350 mM sucrose; and 0.1-1.1 mg/mL polysorbate 80.
N597. 1-150 mg/mL EGFR/LGR5 BsAb; 10-180 mM sodium chloride; 150-350 mM maltose; and 0.1-1.1 mg/mL polysorbate 80.
N598. 10-60 mg/mL EGFR/LGR5 BsAb; 10-180 mM sodium chloride; 150-350 mM maltose; and 0.1-1.1 mg/mL polysorbate 80.
N599. 1-150 mg/mL EGFR/LGR5 BsAb; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N600. 10-60 mg/mL EGFR/LGR5 BsAb; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N601. 1-150 mg/mL EGFR/LGR5 BsAb; 10-180 mM sodium chloride; 150-350 mM sucrose; and 5-150 mM amino acid component selected from the group consisting of proline, glycine, lysine, and any combination thereof.
N602. 10-60 mg/mL EGFR/LGR5 BsAb; 10-180 mM sodium chloride; 150-350 mM sucrose; and 5-150 mM amino acid component selected from the group consisting of proline, glycine, lysine, and any combination thereof.
N603. 1-150 mg/mL EGFR/LGR5 BsAb; 10-180 mM sodium chloride; 150-350 mM maltose; and 5-150 mM amino acid component selected from the group consisting of proline, glycine, lysine, and any combination thereof.
N604. 10-60 mg/mL EGFR/LGR5 BsAb; 10-180 mM sodium chloride; 150-350 mM maltose; and 5-150 mM amino acid component selected from the group consisting of proline, glycine, lysine, and any combination thereof.
N605. 1-150 mg/mL EGFR/LGR5 BsAb; 10-180 mM sodium chloride; 100-400 mM sugar component selected from the group consisting of sucrose, mannitol, sorbitol, and maltose; and 2-25 mM methionine.
N606. 10-60 mg/mL EGFR/LGR5 BsAb; 10-180 mM sodium chloride; 100-400 mM sugar component selected from the group consisting of sucrose, mannitol, sorbitol, and maltose; and 2-25 mM methionine.
N607. 1-150 mg/mL EGFR/LGR5 BsAb; 10-180 mM sodium chloride; 150-350 mM sucrose; and 2-25 mM methionine.
N608. 10-60 mg/mL EGFR/LGR5 BsAb; 10-180 mM sodium chloride; 150-350 mM sucrose; and 2-25 mM methionine.
N609. 1-150 mg/mL EGFR/LGR5 BsAb; 10-180 mM sodium chloride; 150-350 mM maltose; and 2-25 mM methionine.
N610. 10-60 mg/mL EGFR/LGR5 BsAb; 10-180 mM sodium chloride; 150-350 mM maltose; and 2-25 mM methionine.
N611. 1-150 mg/mL EGFR/LGR5 BsAb; 10-180 mM sodium chloride; 100-400 mM sugar component selected from the group consisting of sucrose, mannitol, sorbitol, and maltose; and 0.01-0.2 mM EDTA.
N612. 10-60 mg/mL EGFR/LGR5 BsAb; 10-180 mM sodium chloride; 100-400 mM sugar component selected from the group consisting of sucrose, mannitol, sorbitol, and maltose; and 0.01-0.2 mM EDTA.
N613. 1-150 mg/mL EGFR/LGR5 BsAb; 10-180 mM sodium chloride; 150-350 mM sucrose; and 0.01-0.2 mM EDTA.
N614. 10-60 mg/mL EGFR/LGR5 BsAb; 10-180 mM sodium chloride; 150-350 mM sucrose; and 0.01-0.2 mM EDTA.
N615. 1-150 mg/mL EGFR/LGR5 BsAb; 10-180 mM sodium chloride; 150-350 mM maltose; and 0.01-0.2 mM EDTA.
N616. 10-60 mg/mL EGFR/LGR5 BsAb; 10-180 mM sodium chloride; 150-350 mM maltose; and 0.01-0.2 mM EDTA.
N617. 1-150 mg/mL EGFR/LGR5 BsAb; 10-180 mM sodium chloride; 0.4-1.2 mg/mL surfactant; and 5-150 mM amino acid component selected from the group consisting of proline, glycine, lysine, and any combination thereof.
N618. 10-60 mg/mL EGFR/LGR5 BsAb; 10-180 mM sodium chloride; 0.4-1.2 mg/mL surfactant; and 5-150 mM amino acid component selected from the group consisting of proline, glycine, lysine, and any combination thereof.
N619. 1-150 mg/mL EGFR/LGR5 BsAb; 10-180 mM sodium chloride; 0.1-1.1 mg/mL polysorbate 80; and 5-150 mM amino acid component selected from the group consisting of proline, glycine, lysine, and any combination thereof.
N620. 10-60 mg/mL EGFR/LGR5 BsAb; 10-180 mM sodium chloride; 0.1-1.1 mg/mL polysorbate 80; and 5-150 mM amino acid component selected from the group consisting of proline, glycine, lysine, and any combination thereof.
N621. 1-150 mg/mL EGFR/LGR5 BsAb; 10-180 mM sodium chloride; 0.4-1.2 mg/mL surfactant; and 2-25 mM methionine.
N622. 10-60 mg/mL EGFR/LGR5 BsAb; 10-180 mM sodium chloride; 0.4-1.2 mg/mL surfactant; and 2-25 mM methionine.
N623. 1-150 mg/mL EGFR/LGR5 BsAb; 10-180 mM sodium chloride; 0.1-1.1 mg/mL polysorbate 80; and 2-25 mM methionine.
N624. 10-60 mg/mL EGFR/LGR5 BsAb; 10-180 mM sodium chloride; 0.1-1.1 mg/mL polysorbate 80; and 2-25 mM methionine.
N625. 1-150 mg/mL EGFR/LGR5 BsAb; 10-180 mM sodium chloride; 0.4-1.2 mg/mL surfactant; and 0.01-0.2 mM EDTA.
N626. 10-60 mg/mL EGFR/LGR5 BsAb; 10-180 mM sodium chloride; 0.4-1.2 mg/mL surfactant; and 0.01-0.2 mM EDTA.
N627. 1-150 mg/mL EGFR/LGR5 BsAb; 10-180 mM sodium chloride; 0.1-1.1 mg/mL polysorbate 80; and 0.01-0.2 mM EDTA.
N628. 10-60 mg/mL EGFR/LGR5 BsAb; 10-180 mM sodium chloride; 0.1-1.1 mg/mL polysorbate 80; and 0.01-0.2 mM EDTA.
N629. 1-150 mg/mL EGFR/LGR5 BsAb; 10-180 mM sodium chloride; 5-150 mM amino acid component selected from the group consisting of proline, glycine, lysine, and any combination thereof; and 2-25 mM methionine.
N630. 10-60 mg/mL EGFR/LGR5 BsAb; 10-180 mM sodium chloride; 5-150 mM amino acid component selected from the group consisting of proline, glycine, lysine, and any combination thereof; and 2-25 mM methionine.
N631. 1-150 mg/mL EGFR/LGR5 BsAb; 10-180 mM sodium chloride; 5-150 mM amino acid component selected from the group consisting of proline, glycine, lysine, and any combination thereof; and 0.01-0.2 mM EDTA.
N632. 10-60 mg/mL EGFR/LGR5 BsAb; 10-180 mM sodium chloride; 5-150 mM amino acid component selected from the group consisting of proline, glycine, lysine, and any combination thereof; and 0.01-0.2 mM EDTA.
N633. 1-150 mg/mL EGFR/LGR5 BsAb; 10-180 mM sodium chloride; 2-25 mM methionine; and 0.01-0.2 mM EDTA.
N634. 10-60 mg/mL EGFR/LGR5 BsAb; 10-180 mM sodium chloride; 2-25 mM methionine; and 0.01-0.2 mM EDTA.
N635. 1-150 mg/mL EGFR/LGR5 BsAb; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N636. 10-60 mg/mL EGFR/LGR5 BsAb; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N637. 1-150 mg/mL EGFR/LGR5 BsAb; 150-350 mM sucrose; 0.4-1.2 mg/mL surfactant; and 5-150 mM amino acid component selected from the group consisting of proline, glycine, lysine, and any combination thereof.
N638. 10-60 mg/mL EGFR/LGR5 BsAb; 150-350 mM sucrose; 0.4-1.2 mg/mL surfactant; and 5-150 mM amino acid component selected from the group consisting of proline, glycine, lysine, and any combination thereof.
N639. 1-150 mg/mL EGFR/LGR5 BsAb; 150-350 mM maltose; 0.4-1.2 mg/mL surfactant; and 5-150 mM amino acid component selected from the group consisting of proline, glycine, lysine, and any combination thereof.
N640. 10-60 mg/mL EGFR/LGR5 BsAb; 150-350 mM maltose; 0.4-1.2 mg/mL surfactant; and 5-150 mM amino acid component selected from the group consisting of proline, glycine, lysine, and any combination thereof.
N641. 1-150 mg/mL EGFR/LGR5 BsAb; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N642. 10-60 mg/mL EGFR/LGR5 BsAb; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N643. 1-150 mg/mL EGFR/LGR5 BsAb; 150-350 mM sucrose; 0.1-1.1 mg/mL polysorbate 80; and 5-150 mM amino acid component selected from the group consisting of proline, glycine, lysine, and any combination thereof.
N644. 10-60 mg/mL EGFR/LGR5 BsAb; 150-350 mM sucrose; 0.1-1.1 mg/mL polysorbate 80; and 5-150 mM amino acid component selected from the group consisting of proline, glycine, lysine, and any combination thereof.
N645. 1-150 mg/mL EGFR/LGR5 BsAb; 150-350 mM maltose; 0.1-1.1 mg/mL polysorbate 80; and 5-150 mM amino acid component selected from the group consisting of proline, glycine, lysine, and any combination thereof.
N646. 10-60 mg/mL EGFR/LGR5 BsAb; 150-350 mM maltose; 0.1-1.1 mg/mL polysorbate 80; and 5-150 mM amino acid component selected from the group consisting of proline, glycine, lysine, and any combination thereof.
N647. 1-150 mg/mL EGFR/LGR5 BsAb; 100-400 mM sugar component selected from the group consisting of sucrose, mannitol, sorbitol, and maltose; 0.4-1.2 mg/mL surfactant; and 2-25 mM methionine.
N648. 10-60 mg/mL EGFR/LGR5 BsAb; 100-400 mM sugar component selected from the group consisting of sucrose, mannitol, sorbitol, and maltose; 0.4-1.2 mg/mL surfactant; and 2-25 mM methionine.
N649. 1-150 mg/mL EGFR/LGR5 BsAb; 150-350 mM sucrose; 0.4-1.2 mg/mL surfactant; and 2-25 mM methionine.
N650. 10-60 mg/mL EGFR/LGR5 BsAb; 150-350 mM sucrose; 0.4-1.2 mg/mL surfactant; and 2-25 mM methionine.
N651. 1-150 mg/mL EGFR/LGR5 BsAb; 150-350 mM maltose; 0.4-1.2 mg/mL surfactant; and 2-25 mM methionine.
N652. 10-60 mg/mL EGFR/LGR5 BsAb; 150-350 mM maltose; 0.4-1.2 mg/mL surfactant; and 2-25 mM methionine.
N653. 1-150 mg/mL EGFR/LGR5 BsAb; 100-400 mM sugar component selected from the group consisting of sucrose, mannitol, sorbitol, and maltose; 0.1-1.1 mg/mL polysorbate 80; and 2-25 mM methionine.
N654. 10-60 mg/mL EGFR/LGR5 BsAb; 100-400 mM sugar component selected from the group consisting of sucrose, mannitol, sorbitol, and maltose; 0.1-1.1 mg/mL polysorbate 80; and 2-25 mM methionine.
N655. 1-150 mg/mL EGFR/LGR5 BsAb; 150-350 mM sucrose; 0.1-1.1 mg/mL polysorbate 80; and 2-25 mM methionine.
N656. 10-60 mg/mL EGFR/LGR5 BsAb; 150-350 mM sucrose; 0.1-1.1 mg/mL polysorbate 80; and 2-25 mM methionine.
N657. 1-150 mg/mL EGFR/LGR5 BsAb; 150-350 mM maltose; 0.1-1.1 mg/mL polysorbate 80; and 2-25 mM methionine.
N658. 10-60 mg/mL EGFR/LGR5 BsAb; 150-350 mM maltose; 0.1-1.1 mg/mL polysorbate 80; and 2-25 mM methionine.
N659. 1-150 mg/mL EGFR/LGR5 BsAb; 100-400 mM sugar component selected from the group consisting of sucrose, mannitol, sorbitol, and maltose; 0.4-1.2 mg/mL surfactant; and 0.01-0.2 mM EDTA.
N660. 10-60 mg/mL EGFR/LGR5 BsAb; 100-400 mM sugar component selected from the group consisting of sucrose, mannitol, sorbitol, and maltose; 0.4-1.2 mg/mL surfactant; and 0.01-0.2 mM EDTA.
N661. 1-150 mg/mL EGFR/LGR5 BsAb; 150-350 mM sucrose; 0.4-1.2 mg/mL surfactant; and 0.01-0.2 mM EDTA.
N662. 10-60 mg/mL EGFR/LGR5 BsAb; 150-350 mM sucrose; 0.4-1.2 mg/mL surfactant; and 0.01-0.2 mM EDTA.
N663. 1-150 mg/mL EGFR/LGR5 BsAb; 150-350 mM maltose; 0.4-1.2 mg/mL surfactant; and 0.01-0.2 mM EDTA.
N664. 10-60 mg/mL EGFR/LGR5 BsAb; 150-350 mM maltose; 0.4-1.2 mg/mL surfactant; and 0.01-0.2 mM EDTA.
N665. 1-150 mg/mL EGFR/LGR5 BsAb; 100-400 mM sugar component selected from the group consisting of sucrose, mannitol, sorbitol, and maltose; 0.1-1.1 mg/mL polysorbate 80; and 0.01-0.2 mM EDTA.
N666. 10-60 mg/mL EGFR/LGR5 BsAb; 100-400 mM sugar component selected from the group consisting of sucrose, mannitol, sorbitol, and maltose; 0.1-1.1 mg/mL polysorbate 80; and 0.01-0.2 mM EDTA.
N667. 1-150 mg/mL EGFR/LGR5 BsAb; 150-350 mM sucrose; 0.1-1.1 mg/mL polysorbate 80; and 0.01-0.2 mM EDTA.
N668. 10-60 mg/mL EGFR/LGR5 BsAb; 150-350 mM sucrose; 0.1-1.1 mg/mL polysorbate 80; and 0.01-0.2 mM EDTA.
N669. 1-150 mg/mL EGFR/LGR5 BsAb; 150-350 mM maltose; 0.1-1.1 mg/mL polysorbate 80; and 0.01-0.2 mM EDTA.
N670. 10-60 mg/mL EGFR/LGR5 BsAb; 150-350 mM maltose; 0.1-1.1 mg/mL polysorbate 80; and 0.01-0.2 mM EDTA.
N671. 1-150 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N672. 10-60 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N673. 1-150 mg/mL EGFR/LGR5 BsAb; 150-350 mM sucrose; 5-150 mM amino acid component selected from the group consisting of proline, glycine, lysine, and any combination thereof; and 2-25 mM methionine.
N674. 10-60 mg/mL EGFR/LGR5 BsAb; 150-350 mM sucrose; 5-150 mM amino acid component selected from the group consisting of proline, glycine, lysine, and any combination thereof; and 2-25 mM methionine.
N675. 1-150 mg/mL EGFR/LGR5 BsAb; 150-350 mM maltose; 5-150 mM amino acid component selected from the group consisting of proline, glycine, lysine, and any combination thereof; and 2-25 mM methionine.
N676. 10-60 mg/mL EGFR/LGR5 BsAb; 150-350 mM maltose; 5-150 mM amino acid component selected from the group consisting of proline, glycine, lysine, and any combination thereof; and 2-25 mM methionine.
N677. 1-150 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N678. 10-60 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N679. 1-150 mg/mL EGFR/LGR5 BsAb; 150-350 mM sucrose; 5-150 mM amino acid component selected from the group consisting of proline, glycine, lysine, and any combination thereof; and 0.01-0.2 mM EDTA.
N680. 10-60 mg/mL EGFR/LGR5 BsAb; 150-350 mM sucrose; 5-150 mM amino acid component selected from the group consisting of proline, glycine, lysine, and any combination thereof; and 0.01-0.2 mM EDTA.
N681. 1-150 mg/mL EGFR/LGR5 BsAb; 150-350 mM maltose; 5-150 mM amino acid component selected from the group consisting of proline, glycine, lysine, and any combination thereof; and 0.01-0.2 mM EDTA.
N682. 10-60 mg/mL EGFR/LGR5 BsAb; 150-350 mM maltose; 5-150 mM amino acid component selected from the group consisting of proline, glycine, lysine, and any combination thereof; and 0.01-0.2 mM EDTA.
N683. 1-150 mg/mL EGFR/LGR5 BsAb; 100-400 mM sugar component selected from the group consisting of sucrose, mannitol, sorbitol, and maltose; 2-25 mM methionine; and 0.01-0.2 mM EDTA.
N684. 10-60 mg/mL EGFR/LGR5 BsAb; 100-400 mM sugar component selected from the group consisting of sucrose, mannitol, sorbitol, and maltose; 2-25 mM methionine; and 0.01-0.2 mM EDTA.
N685. 1-150 mg/mL EGFR/LGR5 BsAb; 150-350 mM sucrose; 2-25 mM methionine; and 0.01-0.2 mM EDTA.
N686. 10-60 mg/mL EGFR/LGR5 BsAb; 150-350 mM sucrose; 2-25 mM methionine; and 0.01-0.2 mM EDTA.
N687. 1-150 mg/mL EGFR/LGR5 BsAb; 150-350 mM maltose; 2-25 mM methionine; and 0.01-0.2 mM EDTA.
N688. 10-60 mg/mL EGFR/LGR5 BsAb; 150-350 mM maltose; 2-25 mM methionine; and 0.01-0.2 mM EDTA.
N689. 1-150 mg/mL EGFR/LGR5 BsAb; 0.4-1.2 mg/mL surfactant; 5-150 mM amino acid component selected from the group consisting of proline, glycine, lysine, and any combination thereof; and 2-25 mM methionine.
N690. 10-60 mg/mL EGFR/LGR5 BsAb; 0.4-1.2 mg/mL surfactant; 5-150 mM amino acid component selected from the group consisting of proline, glycine, lysine, and any combination thereof; and 2-25 mM methionine.
N691. 1-150 mg/mL EGFR/LGR5 BsAb; 0.1-1.1 mg/mL polysorbate 80; 5-150 mM amino acid component selected from the group consisting of proline, glycine, lysine, and any combination thereof; and 2-25 mM methionine.
N692. 10-60 mg/mL EGFR/LGR5 BsAb; 0.1-1.1 mg/mL polysorbate 80; 5-150 mM amino acid component selected from the group consisting of proline, glycine, lysine, and any combination thereof; and 2-25 mM methionine.
N693. 1-150 mg/mL EGFR/LGR5 BsAb; 0.4-1.2 mg/mL surfactant; 5-150 mM amino acid component selected from the group consisting of proline, glycine, lysine, and any combination thereof; and 0.01-0.2 mM EDTA.
N694. 10-60 mg/mL EGFR/LGR5 BsAb; 0.4-1.2 mg/mL surfactant; 5-150 mM amino acid component selected from the group consisting of proline, glycine, lysine, and any combination thereof; and 0.01-0.2 mM EDTA.
N695. 1-150 mg/mL EGFR/LGR5 BsAb; 0.1-1.1 mg/mL polysorbate 80; 5-150 mM amino acid component selected from the group consisting of proline, glycine, lysine, and any combination thereof; and 0.01-0.2 mM EDTA.
N696. 10-60 mg/mL EGFR/LGR5 BsAb; 0.1-1.1 mg/mL polysorbate 80; 5-150 mM amino acid component selected from the group consisting of proline, glycine, lysine, and any combination thereof; and 0.01-0.2 mM EDTA.
N697. 1-150 mg/mL EGFR/LGR5 BsAb; 0.4-1.2 mg/mL surfactant; 2-25 mM methionine; and 0.01-0.2 mM EDTA.
N698. 10-60 mg/mL EGFR/LGR5 BsAb; 0.4-1.2 mg/mL surfactant; 2-25 mM methionine; and 0.01-0.2 mM EDTA.
N699. 1-150 mg/mL EGFR/LGR5 BsAb; 0.1-1.1 mg/mL polysorbate 80; 2-25 mM methionine; and 0.01-0.2 mM EDTA.
N700. 10-60 mg/mL EGFR/LGR5 BsAb; 0.1-1.1 mg/mL polysorbate 80; 2-25 mM methionine; and 0.01-0.2 mM EDTA.
N701. 1-150 mg/mL EGFR/LGR5 BsAb; 5-150 mM amino acid component selected from the group consisting of proline, glycine, lysine, and any combination thereof; 2-25 mM methionine; and 0.01-0.2 mM EDTA.
N702. 10-60 mg/mL EGFR/LGR5 BsAb; 5-150 mM amino acid component selected from the group consisting of proline, glycine, lysine, and any combination thereof; 2-25 mM methionine; and 0.01-0.2 mM EDTA.
N703. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.4-1.2 mg/mL surfactant.
N704. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.4-1.2 mg/mL surfactant.
N705. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.4-1.2 mg/mL surfactant.
N706. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.4-1.2 mg/mL surfactant.
N707. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.4-1.2 mg/mL surfactant.
N708. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.4-1.2 mg/mL surfactant.
N709. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.4-1.2 mg/mL surfactant.
N710. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.4-1.2 mg/mL surfactant.
N711. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.4-1.2 mg/mL surfactant.
N712. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.4-1.2 mg/mL surfactant.
N713. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.4-1.2 mg/mL surfactant.
N714. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.4-1.2 mg/mL surfactant.
N715. 1-150 mg/mL EGFR/LGR5 BsAb; 3-25 mM histidine buffer system providing a composition pH of 5.4-7.1; 10-180 mM sodium chloride; 150-350 mM sucrose; and 0.4-1.2 mg/mL surfactant.
N716. 10-60 mg/mL EGFR/LGR5 BsAb; 3-25 mM histidine buffer system providing a composition pH of 5.4-7.1; 10-180 mM sodium chloride; 150-350 mM sucrose; and 0.4-1.2 mg/mL surfactant.
N717. 1-150 mg/mL EGFR/LGR5 BsAb; 3-25 mM acetate buffer system providing a composition pH of 4-5.6; 10-180 mM sodium chloride; 150-350 mM sucrose; and 0.4-1.2 mg/mL surfactant.
N718. 10-60 mg/mL EGFR/LGR5 BsAb; 3-25 mM acetate buffer system providing a composition pH of 4-5.6; 10-180 mM sodium chloride; 150-350 mM sucrose; and 0.4-1.2 mg/mL surfactant.
N719. 1-150 mg/mL EGFR/LGR5 BsAb; 3-25 mM citrate buffer system providing a composition pH of 4.5-7.5; 10-180 mM sodium chloride; 150-350 mM sucrose; and 0.4-1.2 mg/mL surfactant.
N720. 10-60 mg/mL EGFR/LGR5 BsAb; 3-25 mM citrate buffer system providing a composition pH of 4.5-7.5; 10-180 mM sodium chloride; 150-350 mM sucrose; and 0.4-1.2 mg/mL surfactant.
N721. 1-150 mg/mL EGFR/LGR5 BsAb; 3-25 mM succinate buffer system providing a composition pH of 4-6.6; 10-180 mM sodium chloride; 150-350 mM sucrose; and 0.4-1.2 mg/mL surfactant.
N722. 10-60 mg/mL EGFR/LGR5 BsAb; 3-25 mM succinate buffer system providing a composition pH of 4-6.6; 10-180 mM sodium chloride; 150-350 mM sucrose; and 0.4-1.2 mg/mL surfactant.
N723. 1-150 mg/mL EGFR/LGR5 BsAb; 3-25 mM phosphate buffer system providing a composition pH of 5.8-8.1; 10-180 mM sodium chloride; 150-350 mM sucrose; and 0.4-1.2 mg/mL surfactant.
N724. 10-60 mg/mL EGFR/LGR5 BsAb; 3-25 mM phosphate buffer system providing a composition pH of 5.8-8.1; 10-180 mM sodium chloride; 150-350 mM sucrose; and 0.4-1.2 mg/mL surfactant.
N725. 1-150 mg/mL EGFR/LGR5 BsAb; 3-25 mM citrate-phosphate buffer system providing a composition pH of 4-7.1; 10-180 mM sodium chloride; 150-350 mM sucrose; and 0.4-1.2 mg/mL surfactant.
N726. 10-60 mg/mL EGFR/LGR5 BsAb; 3-25 mM citrate-phosphate buffer system providing a composition pH of 4-7.1; 10-180 mM sodium chloride; 150-350 mM sucrose; and 0.4-1.2 mg/mL surfactant.
N727. 1-150 mg/mL EGFR/LGR5 BsAb; 3-25 mM histidine buffer system providing a composition pH of 5.4-7.1; 10-180 mM sodium chloride; 150-350 mM maltose; and 0.4-1.2 mg/mL surfactant.
N728. 10-60 mg/mL EGFR/LGR5 BsAb; 3-25 mM histidine buffer system providing a composition pH of 5.4-7.1; 10-180 mM sodium chloride; 150-350 mM maltose; and 0.4-1.2 mg/mL surfactant.
N729. 1-150 mg/mL EGFR/LGR5 BsAb; 3-25 mM acetate buffer system providing a composition pH of 4-5.6; 10-180 mM sodium chloride; 150-350 mM maltose; and 0.4-1.2 mg/mL surfactant.
N730. 10-60 mg/mL EGFR/LGR5 BsAb; 3-25 mM acetate buffer system providing a composition pH of 4-5.6; 10-180 mM sodium chloride; 150-350 mM maltose; and 0.4-1.2 mg/mL surfactant.
N731. 1-150 mg/mL EGFR/LGR5 BsAb; 3-25 mM citrate buffer system providing a composition pH of 4.5-7.5; 10-180 mM sodium chloride; 150-350 mM maltose; and 0.4-1.2 mg/mL surfactant.
N732. 10-60 mg/mL EGFR/LGR5 BsAb; 3-25 mM citrate buffer system providing a composition pH of 4.5-7.5; 10-180 mM sodium chloride; 150-350 mM maltose; and 0.4-1.2 mg/mL surfactant.
N733. 1-150 mg/mL EGFR/LGR5 BsAb; 3-25 mM succinate buffer system providing a composition pH of 4-6.6; 10-180 mM sodium chloride; 150-350 mM maltose; and 0.4-1.2 mg/mL surfactant.
N734. 10-60 mg/mL EGFR/LGR5 BsAb; 3-25 mM succinate buffer system providing a composition pH of 4-6.6; 10-180 mM sodium chloride; 150-350 mM maltose; and 0.4-1.2 mg/mL surfactant.
N735. 1-150 mg/mL EGFR/LGR5 BsAb; 3-25 mM phosphate buffer system providing a composition pH of 5.8-8.1; 10-180 mM sodium chloride; 150-350 mM maltose; and 0.4-1.2 mg/mL surfactant.
N736. 10-60 mg/mL EGFR/LGR5 BsAb; 3-25 mM phosphate buffer system providing a composition pH of 5.8-8.1; 10-180 mM sodium chloride; 150-350 mM maltose; and 0.4-1.2 mg/mL surfactant.
N737. 1-150 mg/mL EGFR/LGR5 BsAb; 3-25 mM citrate-phosphate buffer system providing a composition pH of 4-7.1; 10-180 mM sodium chloride; 150-350 mM maltose; and 0.4-1.2 mg/mL surfactant.
N738. 10-60 mg/mL EGFR/LGR5 BsAb; 3-25 mM citrate-phosphate buffer system providing a composition pH of 4-7.1; 10-180 mM sodium chloride; 150-350 mM maltose; and 0.4-1.2 mg/mL surfactant.
N739. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.1-1.1 mg/mL polysorbate 80.
N740. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.1-1.1 mg/mL polysorbate 80.
N741. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.1-1.1 mg/mL polysorbate 80.
N742. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.1-1.1 mg/mL polysorbate 80.
N743. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.1-1.1 mg/mL polysorbate 80.
N744. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.1-1.1 mg/mL polysorbate 80.
N745. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.1-1.1 mg/mL polysorbate 80.
N746. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.1-1.1 mg/mL polysorbate 80.
N747. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.1-1.1 mg/mL polysorbate 80.
N748. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.1-1.1 mg/mL polysorbate 80.
N749. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.1-1.1 mg/mL polysorbate 80.
N750. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.1-1.1 mg/mL polysorbate 80.
N751. 1-150 mg/mL EGFR/LGR5 BsAb; 3-25 mM histidine buffer system providing a composition pH of 5.4-7.1; 10-180 mM sodium chloride; 150-350 mM sucrose; and 0.1-1.1 mg/mL polysorbate 80.
N752. 10-60 mg/mL EGFR/LGR5 BsAb; 3-25 mM histidine buffer system providing a composition pH of 5.4-7.1; 10-180 mM sodium chloride; 150-350 mM sucrose; and 0.1-1.1 mg/mL polysorbate 80.
N753. 1-150 mg/mL EGFR/LGR5 BsAb; 3-25 mM acetate buffer system providing a composition pH of 4-5.6; 10-180 mM sodium chloride; 150-350 mM sucrose; and 0.1-1.1 mg/mL polysorbate 80.
N754. 10-60 mg/mL EGFR/LGR5 BsAb; 3-25 mM acetate buffer system providing a composition pH of 4-5.6; 10-180 mM sodium chloride; 150-350 mM sucrose; and 0.1-1.1 mg/mL polysorbate 80.
N755. 1-150 mg/mL EGFR/LGR5 BsAb; 3-25 mM citrate buffer system providing a composition pH of 4.5-7.5; 10-180 mM sodium chloride; 150-350 mM sucrose; and 0.1-1.1 mg/mL polysorbate 80.
N756. 10-60 mg/mL EGFR/LGR5 BsAb; 3-25 mM citrate buffer system providing a composition pH of 4.5-7.5; 10-180 mM sodium chloride; 150-350 mM sucrose; and 0.1-1.1 mg/mL polysorbate 80.
N757. 1-150 mg/mL EGFR/LGR5 BsAb; 3-25 mM succinate buffer system providing a composition pH of 4-6.6; 10-180 mM sodium chloride; 150-350 mM sucrose; and 0.1-1.1 mg/mL polysorbate 80.
N758. 10-60 mg/mL EGFR/LGR5 BsAb; 3-25 mM succinate buffer system providing a composition pH of 4-6.6; 10-180 mM sodium chloride; 150-350 mM sucrose; and 0.1-1.1 mg/mL polysorbate 80.
N759. 1-150 mg/mL EGFR/LGR5 BsAb; 3-25 mM phosphate buffer system providing a composition pH of 5.8-8.1; 10-180 mM sodium chloride; 150-350 mM sucrose; and 0.1-1.1 mg/mL polysorbate 80.
N760. 10-60 mg/mL EGFR/LGR5 BsAb; 3-25 mM phosphate buffer system providing a composition pH of 5.8-8.1; 10-180 mM sodium chloride; 150-350 mM sucrose; and 0.1-1.1 mg/mL polysorbate 80.
N761. 1-150 mg/mL EGFR/LGR5 BsAb; 3-25 mM citrate-phosphate buffer system providing a composition pH of 4-7.1; 10-180 mM sodium chloride; 150-350 mM sucrose; and 0.1-1.1 mg/mL polysorbate 80.
N762. 10-60 mg/mL EGFR/LGR5 BsAb; 3-25 mM citrate-phosphate buffer system providing a composition pH of 4-7.1; 10-180 mM sodium chloride; 150-350 mM sucrose; and 0.1-1.1 mg/mL polysorbate 80.
N763. 1-150 mg/mL EGFR/LGR5 BsAb; 3-25 mM histidine buffer system providing a composition pH of 5.4-7.1; 10-180 mM sodium chloride; 150-350 mM maltose; and 0.1-1.1 mg/mL polysorbate 80.
N764. 10-60 mg/mL EGFR/LGR5 BsAb; 3-25 mM histidine buffer system providing a composition pH of 5.4-7.1; 10-180 mM sodium chloride; 150-350 mM maltose; and 0.1-1.1 mg/mL polysorbate 80.
N765. 1-150 mg/mL EGFR/LGR5 BsAb; 3-25 mM acetate buffer system providing a composition pH of 4-5.6; 10-180 mM sodium chloride; 150-350 mM maltose; and 0.1-1.1 mg/mL polysorbate 80.
N766. 10-60 mg/mL EGFR/LGR5 BsAb; 3-25 mM acetate buffer system providing a composition pH of 4-5.6; 10-180 mM sodium chloride; 150-350 mM maltose; and 0.1-1.1 mg/mL polysorbate 80.
N767. 1-150 mg/mL EGFR/LGR5 BsAb; 3-25 mM citrate buffer system providing a composition pH of 4.5-7.5; 10-180 mM sodium chloride; 150-350 mM maltose; and 0.1-1.1 mg/mL polysorbate 80.
N768. 10-60 mg/mL EGFR/LGR5 BsAb; 3-25 mM citrate buffer system providing a composition pH of 4.5-7.5; 10-180 mM sodium chloride; 150-350 mM maltose; and 0.1-1.1 mg/mL polysorbate 80.
N769. 1-150 mg/mL EGFR/LGR5 BsAb; 3-25 mM succinate buffer system providing a composition pH of 4-6.6; 10-180 mM sodium chloride; 150-350 mM maltose; and 0.1-1.1 mg/mL polysorbate 80.
N770. 10-60 mg/mL EGFR/LGR5 BsAb; 3-25 mM succinate buffer system providing a composition pH of 4-6.6; 10-180 mM sodium chloride; 150-350 mM maltose; and 0.1-1.1 mg/mL polysorbate 80.
N771. 1-150 mg/mL EGFR/LGR5 BsAb; 3-25 mM phosphate buffer system providing a composition pH of 5.8-8.1; 10-180 mM sodium chloride; 150-350 mM maltose; and 0.1-1.1 mg/mL polysorbate 80.
N772. 10-60 mg/mL EGFR/LGR5 BsAb; 3-25 mM phosphate buffer system providing a composition pH of 5.8-8.1; 10-180 mM sodium chloride; 150-350 mM maltose; and 0.1-1.1 mg/mL polysorbate 80.
N773. 1-150 mg/mL EGFR/LGR5 BsAb; 3-25 mM citrate-phosphate buffer system providing a composition pH of 4-7.1; 10-180 mM sodium chloride; 150-350 mM maltose; and 0.1-1.1 mg/mL polysorbate 80.
N774. 10-60 mg/mL EGFR/LGR5 BsAb; 3-25 mM citrate-phosphate buffer system providing a composition pH of 4-7.1; 10-180 mM sodium chloride; 150-350 mM maltose; and 0.1-1.1 mg/mL polysorbate 80.
N775. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N776. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N777. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N778. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N779. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N780. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N781. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N782. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N783. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N784. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N785. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N786. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 5-150 mM amino acid component selected from the group consisting of proline, glycine, lysine, and any combination thereof.
N787. 1-150 mg/mL EGFR/LGR5 BsAb; 3-25 mM histidine buffer system providing a composition pH of 5.4-7.1; 10-180 mM sodium chloride; 150-350 mM sucrose; and 5-150 mM amino acid component selected from the group consisting of proline, glycine, lysine, and any combination thereof.
N788. 10-60 mg/mL EGFR/LGR5 BsAb; 3-25 mM histidine buffer system providing a composition pH of 5.4-7.1; 10-180 mM sodium chloride; 150-350 mM sucrose; and 5-150 mM amino acid component selected from the group consisting of proline, glycine, lysine, and any combination thereof.
N789. 1-150 mg/mL EGFR/LGR5 BsAb; 3-25 mM acetate buffer system providing a composition pH of 4-5.6; 10-180 mM sodium chloride; 150-350 mM sucrose; and 5-150 mM amino acid component selected from the group consisting of proline, glycine, lysine, and any combination thereof.
N790. 10-60 mg/mL EGFR/LGR5 BsAb; 3-25 mM acetate buffer system providing a composition pH of 4-5.6; 10-180 mM sodium chloride; 150-350 mM sucrose; and 5-150 mM amino acid component selected from the group consisting of proline, glycine, lysine, and any combination thereof.
N791. 1-150 mg/mL EGFR/LGR5 BsAb; 3-25 mM citrate buffer system providing a composition pH of 4.5-7.5; 10-180 mM sodium chloride; 150-350 mM sucrose; and 5-150 mM amino acid component selected from the group consisting of proline, glycine, lysine, and any combination thereof.
N792. 10-60 mg/mL EGFR/LGR5 BsAb; 3-25 mM citrate buffer system providing a composition pH of 4.5-7.5; 10-180 mM sodium chloride; 150-350 mM sucrose; and 5-150 mM amino acid component selected from the group consisting of proline, glycine, lysine, and any combination thereof.
N793. 1-150 mg/mL EGFR/LGR5 BsAb; 3-25 mM succinate buffer system providing a composition pH of 4-6.6; 10-180 mM sodium chloride; 150-350 mM sucrose; and 5-150 mM amino acid component selected from the group consisting of proline, glycine, lysine, and any combination thereof.
N794. 10-60 mg/mL EGFR/LGR5 BsAb; 3-25 mM succinate buffer system providing a composition pH of 4-6.6; 10-180 mM sodium chloride; 150-350 mM sucrose; and 5-150 mM amino acid component selected from the group consisting of proline, glycine, lysine, and any combination thereof.
N795. 1-150 mg/mL EGFR/LGR5 BsAb; 3-25 mM phosphate buffer system providing a composition pH of 5.8-8.1; 10-180 mM sodium chloride; 150-350 mM sucrose; and 5-150 mM amino acid component selected from the group consisting of proline, glycine, lysine, and any combination thereof.
N796. 10-60 mg/mL EGFR/LGR5 BsAb; 3-25 mM phosphate buffer system providing a composition pH of 5.8-8.1; 10-180 mM sodium chloride; 150-350 mM sucrose; and 5-150 mM amino acid component selected from the group consisting of proline, glycine, lysine, and any combination thereof.
N797. 1-150 mg/mL EGFR/LGR5 BsAb; 3-25 mM citrate-phosphate buffer system providing a composition pH of 4-7.1; 10-180 mM sodium chloride; 150-350 mM sucrose; and 5-150 mM amino acid component selected from the group consisting of proline, glycine, lysine, and any combination thereof.
N798. 10-60 mg/mL EGFR/LGR5 BsAb; 3-25 mM citrate-phosphate buffer system providing a composition pH of 4-7.1; 10-180 mM sodium chloride; 150-350 mM sucrose; and 5-150 mM amino acid component selected from the group consisting of proline, glycine, lysine, and any combination thereof.
N799. 1-150 mg/mL EGFR/LGR5 BsAb; 3-25 mM histidine buffer system providing a composition pH of 5.4-7.1; 10-180 mM sodium chloride; 150-350 mM maltose; and 5-150 mM amino acid component selected from the group consisting of proline, glycine, lysine, and any combination thereof.
N800. 10-60 mg/mL EGFR/LGR5 BsAb; 3-25 mM histidine buffer system providing a composition pH of 5.4-7.1; 10-180 mM sodium chloride; 150-350 mM maltose; and 5-150 mM amino acid component selected from the group consisting of proline, glycine, lysine, and any combination thereof.
N801. 1-150 mg/mL EGFR/LGR5 BsAb; 3-25 mM acetate buffer system providing a composition pH of 4-5.6; 10-180 mM sodium chloride; 150-350 mM maltose; and 5-150 mM amino acid component selected from the group consisting of proline, glycine, lysine, and any combination thereof.
N802. 10-60 mg/mL EGFR/LGR5 BsAb; 3-25 mM acetate buffer system providing a composition pH of 4-5.6; 10-180 mM sodium chloride; 150-350 mM maltose; and 5-150 mM amino acid component selected from the group consisting of proline, glycine, lysine, and any combination thereof.
N803. 1-150 mg/mL EGFR/LGR5 BsAb; 3-25 mM citrate buffer system providing a composition pH of 4.5-7.5; 10-180 mM sodium chloride; 150-350 mM maltose; and 5-150 mM amino acid component selected from the group consisting of proline, glycine, lysine, and any combination thereof.
N804. 10-60 mg/mL EGFR/LGR5 BsAb; 3-25 mM citrate buffer system providing a composition pH of 4.5-7.5; 10-180 mM sodium chloride; 150-350 mM maltose; and 5-150 mM amino acid component selected from the group consisting of proline, glycine, lysine, and any combination thereof.
N805. 1-150 mg/mL EGFR/LGR5 BsAb; 3-25 mM succinate buffer system providing a composition pH of 4-6.6; 10-180 mM sodium chloride; 150-350 mM maltose; and 5-150 mM amino acid component selected from the group consisting of proline, glycine, lysine, and any combination thereof.
N806. 10-60 mg/mL EGFR/LGR5 BsAb; 3-25 mM succinate buffer system providing a composition pH of 4-6.6; 10-180 mM sodium chloride; 150-350 mM maltose; and 5-150 mM amino acid component selected from the group consisting of proline, glycine, lysine, and any combination thereof.
N807. 1-150 mg/mL EGFR/LGR5 BsAb; 3-25 mM phosphate buffer system providing a composition pH of 5.8-8.1; 10-180 mM sodium chloride; 150-350 mM maltose; and 5-150 mM amino acid component selected from the group consisting of proline, glycine, lysine, and any combination thereof.
N808. 10-60 mg/mL EGFR/LGR5 BsAb; 3-25 mM phosphate buffer system providing a composition pH of 5.8-8.1; 10-180 mM sodium chloride; 150-350 mM maltose; and 5-150 mM amino acid component selected from the group consisting of proline, glycine, lysine, and any combination thereof.
N809. 1-150 mg/mL EGFR/LGR5 BsAb; 3-25 mM citrate-phosphate buffer system providing a composition pH of 4-7.1; 10-180 mM sodium chloride; 150-350 mM maltose; and 5-150 mM amino acid component selected from the group consisting of proline, glycine, lysine, and any combination thereof.
N810. 10-60 mg/mL EGFR/LGR5 BsAb; 3-25 mM citrate-phosphate buffer system providing a composition pH of 4-7.1; 10-180 mM sodium chloride; 150-350 mM maltose; and 5-150 mM amino acid component selected from the group consisting of proline, glycine, lysine, and any combination thereof.
N811. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 2-25 mM methionine.
N812. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 2-25 mM methionine.
N813. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 2-25 mM methionine.
N814. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 2-25 mM methionine.
N815. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 2-25 mM methionine.
N816. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 2-25 mM methionine.
N817. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 2-25 mM methionine.
N818. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 2-25 mM methionine.
N819. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 2-25 mM methionine.
N820. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 2-25 mM methionine.
N821. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 2-25 mM methionine.
N822. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 2-25 mM methionine.
N823. 1-150 mg/mL EGFR/LGR5 BsAb; 3-25 mM histidine buffer system providing a composition pH of 5.4-7.1; 10-180 mM sodium chloride; 150-350 mM sucrose; and 2-25 mM methionine.
N824. 10-60 mg/mL EGFR/LGR5 BsAb; 3-25 mM histidine buffer system providing a composition pH of 5.4-7.1; 10-180 mM sodium chloride; 150-350 mM sucrose; and 2-25 mM methionine.
N825. 1-150 mg/mL EGFR/LGR5 BsAb; 3-25 mM acetate buffer system providing a composition pH of 4-5.6; 10-180 mM sodium chloride; 150-350 mM sucrose; and 2-25 mM methionine.
N826. 10-60 mg/mL EGFR/LGR5 BsAb; 3-25 mM acetate buffer system providing a composition pH of 4-5.6; 10-180 mM sodium chloride; 150-350 mM sucrose; and 2-25 mM methionine.
N827. 1-150 mg/mL EGFR/LGR5 BsAb; 3-25 mM citrate buffer system providing a composition pH of 4.5-7.5; 10-180 mM sodium chloride; 150-350 mM sucrose; and 2-25 mM methionine.
N828. 10-60 mg/mL EGFR/LGR5 BsAb; 3-25 mM citrate buffer system providing a composition pH of 4.5-7.5; 10-180 mM sodium chloride; 150-350 mM sucrose; and 2-25 mM methionine.
N829. 1-150 mg/mL EGFR/LGR5 BsAb; 3-25 mM succinate buffer system providing a composition pH of 4-6.6; 10-180 mM sodium chloride; 150-350 mM sucrose; and 2-25 mM methionine.
N830. 10-60 mg/mL EGFR/LGR5 BsAb; 3-25 mM succinate buffer system providing a composition pH of 4-6.6; 10-180 mM sodium chloride; 150-350 mM sucrose; and 2-25 mM methionine.
N831. 1-150 mg/mL EGFR/LGR5 BsAb; 3-25 mM phosphate buffer system providing a composition pH of 5.8-8.1; 10-180 mM sodium chloride; 150-350 mM sucrose; and 2-25 mM methionine.
N832. 10-60 mg/mL EGFR/LGR5 BsAb; 3-25 mM phosphate buffer system providing a composition pH of 5.8-8.1; 10-180 mM sodium chloride; 150-350 mM sucrose; and 2-25 mM methionine.
N833. 1-150 mg/mL EGFR/LGR5 BsAb; 3-25 mM citrate-phosphate buffer system providing a composition pH of 4-7.1; 10-180 mM sodium chloride; 150-350 mM sucrose; and 2-25 mM methionine.
N834. 10-60 mg/mL EGFR/LGR5 BsAb; 3-25 mM citrate-phosphate buffer system providing a composition pH of 4-7.1; 10-180 mM sodium chloride; 150-350 mM sucrose; and 2-25 mM methionine.
N835. 1-150 mg/mL EGFR/LGR5 BsAb; 3-25 mM histidine buffer system providing a composition pH of 5.4-7.1; 10-180 mM sodium chloride; 150-350 mM maltose; and 2-25 mM methionine.
N836. 10-60 mg/mL EGFR/LGR5 BsAb; 3-25 mM histidine buffer system providing a composition pH of 5.4-7.1; 10-180 mM sodium chloride; 150-350 mM maltose; and 2-25 mM methionine.
N837. 1-150 mg/mL EGFR/LGR5 BsAb; 3-25 mM acetate buffer system providing a composition pH of 4-5.6; 10-180 mM sodium chloride; 150-350 mM maltose; and 2-25 mM methionine.
N838. 10-60 mg/mL EGFR/LGR5 BsAb; 3-25 mM acetate buffer system providing a composition pH of 4-5.6; 10-180 mM sodium chloride; 150-350 mM maltose; and 2-25 mM methionine.
N839. 1-150 mg/mL EGFR/LGR5 BsAb; 3-25 mM citrate buffer system providing a composition pH of 4.5-7.5; 10-180 mM sodium chloride; 150-350 mM maltose; and 2-25 mM methionine.
N840. 10-60 mg/mL EGFR/LGR5 BsAb; 3-25 mM citrate buffer system providing a composition pH of 4.5-7.5; 10-180 mM sodium chloride; 150-350 mM maltose; and 2-25 mM methionine.
N841. 1-150 mg/mL EGFR/LGR5 BsAb; 3-25 mM succinate buffer system providing a composition pH of 4-6.6; 10-180 mM sodium chloride; 150-350 mM maltose; and 2-25 mM methionine.
N842. 10-60 mg/mL EGFR/LGR5 BsAb; 3-25 mM succinate buffer system providing a composition pH of 4-6.6; 10-180 mM sodium chloride; 150-350 mM maltose; and 2-25 mM methionine.
N843. 1-150 mg/mL EGFR/LGR5 BsAb; 3-25 mM phosphate buffer system providing a composition pH of 5.8-8.1; 10-180 mM sodium chloride; 150-350 mM maltose; and 2-25 mM methionine.
N844. 10-60 mg/mL EGFR/LGR5 BsAb; 3-25 mM phosphate buffer system providing a composition pH of 5.8-8.1; 10-180 mM sodium chloride; 150-350 mM maltose; and 2-25 mM methionine.
N845. 1-150 mg/mL EGFR/LGR5 BsAb; 3-25 mM citrate-phosphate buffer system providing a composition pH of 4-7.1; 10-180 mM sodium chloride; 150-350 mM maltose; and 2-25 mM methionine.
N846. 10-60 mg/mL EGFR/LGR5 BsAb; 3-25 mM citrate-phosphate buffer system providing a composition pH of 4-7.1; 10-180 mM sodium chloride; 150-350 mM maltose; and 2-25 mM methionine.
N847. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.01-0.2 mM EDTA.
N848. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; and 0.01-0.2 mM EDTA.
N849. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.01-0.2 mM EDTA.
N850. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; and 0.01-0.2 mM EDTA.
N851. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.01-0.2 mM EDTA.
N852. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; and 0.01-0.2 mM EDTA.
N853. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.01-0.2 mM EDTA.
N854. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; and 0.01-0.2 mM EDTA.
N855. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.01-0.2 mM EDTA.
N856. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; and 0.01-0.2 mM EDTA.
N857. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.01-0.2 mM EDTA.
N858. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; and 0.01-0.2 mM EDTA.
N859. 1-150 mg/mL EGFR/LGR5 BsAb; 3-25 mM histidine buffer system providing a composition pH of 5.4-7.1; 10-180 mM sodium chloride; 150-350 mM sucrose; and 0.01-0.2 mM EDTA.
N860. 10-60 mg/mL EGFR/LGR5 BsAb; 3-25 mM histidine buffer system providing a composition pH of 5.4-7.1; 10-180 mM sodium chloride; 150-350 mM sucrose; and 0.01-0.2 mM EDTA.
N861. 1-150 mg/mL EGFR/LGR5 BsAb; 3-25 mM acetate buffer system providing a composition pH of 4-5.6; 10-180 mM sodium chloride; 150-350 mM sucrose; and 0.01-0.2 mM EDTA.
N862. 10-60 mg/mL EGFR/LGR5 BsAb; 3-25 mM acetate buffer system providing a composition pH of 4-5.6; 10-180 mM sodium chloride; 150-350 mM sucrose; and 0.01-0.2 mM EDTA.
N863. 1-150 mg/mL EGFR/LGR5 BsAb; 3-25 mM citrate buffer system providing a composition pH of 4.5-7.5; 10-180 mM sodium chloride; 150-350 mM sucrose; and 0.01-0.2 mM EDTA.
N864. 10-60 mg/mL EGFR/LGR5 BsAb; 3-25 mM citrate buffer system providing a composition pH of 4.5-7.5; 10-180 mM sodium chloride; 150-350 mM sucrose; and 0.01-0.2 mM EDTA.
N865. 1-150 mg/mL EGFR/LGR5 BsAb; 3-25 mM succinate buffer system providing a composition pH of 4-6.6; 10-180 mM sodium chloride; 150-350 mM sucrose; and 0.01-0.2 mM EDTA.
N866. 10-60 mg/mL EGFR/LGR5 BsAb; 3-25 mM succinate buffer system providing a composition pH of 4-6.6; 10-180 mM sodium chloride; 150-350 mM sucrose; and 0.01-0.2 mM EDTA.
N867. 1-150 mg/mL EGFR/LGR5 BsAb; 3-25 mM phosphate buffer system providing a composition pH of 5.8-8.1; 10-180 mM sodium chloride; 150-350 mM sucrose; and 0.01-0.2 mM EDTA.
N868. 10-60 mg/mL EGFR/LGR5 BsAb; 3-25 mM phosphate buffer system providing a composition pH of 5.8-8.1; 10-180 mM sodium chloride; 150-350 mM sucrose; and 0.01-0.2 mM EDTA.
N869. 1-150 mg/mL EGFR/LGR5 BsAb; 3-25 mM citrate-phosphate buffer system providing a composition pH of 4-7.1; 10-180 mM sodium chloride; 150-350 mM sucrose; and 0.01-0.2 mM EDTA.
N870. 10-60 mg/mL EGFR/LGR5 BsAb; 3-25 mM citrate-phosphate buffer system providing a composition pH of 4-7.1; 10-180 mM sodium chloride; 150-350 mM sucrose; and 0.01-0.2 mM EDTA.
N871. 1-150 mg/mL EGFR/LGR5 BsAb; 3-25 mM histidine buffer system providing a composition pH of 5.4-7.1; 10-180 mM sodium chloride; 150-350 mM maltose; and 0.01-0.2 mM EDTA.
N872. 10-60 mg/mL EGFR/LGR5 BsAb; 3-25 mM histidine buffer system providing a composition pH of 5.4-7.1; 10-180 mM sodium chloride; 150-350 mM maltose; and 0.01-0.2 mM EDTA.
N873. 1-150 mg/mL EGFR/LGR5 BsAb; 3-25 mM acetate buffer system providing a composition pH of 4-5.6; 10-180 mM sodium chloride; 150-350 mM maltose; and 0.01-0.2 mM EDTA.
N874. 10-60 mg/mL EGFR/LGR5 BsAb; 3-25 mM acetate buffer system providing a composition pH of 4-5.6; 10-180 mM sodium chloride; 150-350 mM maltose; and 0.01-0.2 mM EDTA.
N875. 1-150 mg/mL EGFR/LGR5 BsAb; 3-25 mM citrate buffer system providing a composition pH of 4.5-7.5; 10-180 mM sodium chloride; 150-350 mM maltose; and 0.01-0.2 mM EDTA.
N876. 10-60 mg/mL EGFR/LGR5 BsAb; 3-25 mM citrate buffer system providing a composition pH of 4.5-7.5; 10-180 mM sodium chloride; 150-350 mM maltose; and 0.01-0.2 mM EDTA.
N877. 1-150 mg/mL EGFR/LGR5 BsAb; 3-25 mM succinate buffer system providing a composition pH of 4-6.6; 10-180 mM sodium chloride; 150-350 mM maltose; and 0.01-0.2 mM EDTA.
N878. 10-60 mg/mL EGFR/LGR5 BsAb; 3-25 mM succinate buffer system providing a composition pH of 4-6.6; 10-180 mM sodium chloride; 150-350 mM maltose; and 0.01-0.2 mM EDTA.
N879. 1-150 mg/mL EGFR/LGR5 BsAb; 3-25 mM phosphate buffer system providing a composition pH of 5.8-8.1; 10-180 mM sodium chloride; 150-350 mM maltose; and 0.01-0.2 mM EDTA.
N880. 10-60 mg/mL EGFR/LGR5 BsAb; 3-25 mM phosphate buffer system providing a composition pH of 5.8-8.1; 10-180 mM sodium chloride; 150-350 mM maltose; and 0.01-0.2 mM EDTA.
N881. 1-150 mg/mL EGFR/LGR5 BsAb; 3-25 mM citrate-phosphate buffer system providing a composition pH of 4-7.1; 10-180 mM sodium chloride; 150-350 mM maltose; and 0.01-0.2 mM EDTA.
N882. 10-60 mg/mL EGFR/LGR5 BsAb; 3-25 mM citrate-phosphate buffer system providing a composition pH of 4-7.1; 10-180 mM sodium chloride; 150-350 mM maltose; and 0.01-0.2 mM EDTA.
N883. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; and 5-150 mM amino acid component selected from the group consisting of proline, glycine, lysine, and any combination thereof.
N884. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; and 5-150 mM amino acid component selected from the group consisting of proline, glycine, lysine, and any combination thereof.
N885. 1-150 mg/mL EGFR/LGR5 BsAb; 3-25 mM acetate buffer system providing a composition pH of 4-5.6; 10-180 mM sodium chloride; 0.4-1.2 mg/mL surfactant; and 5-150 mM amino acid component selected from the group consisting of proline, glycine, lysine, and any combination thereof.
N886. 10-60 mg/mL EGFR/LGR5 BsAb; 3-25 mM acetate buffer system providing a composition pH of 4-5.6; 10-180 mM sodium chloride; 0.4-1.2 mg/mL surfactant; and 5-150 mM amino acid component selected from the group consisting of proline, glycine, lysine, and any combination thereof.
N887. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; and 5-150 mM amino acid component selected from the group consisting of proline, glycine, lysine, and any combination thereof.
N888. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; and 5-150 mM amino acid component selected from the group consisting of proline, glycine, lysine, and any combination thereof.
N889. 1-150 mg/mL EGFR/LGR5 BsAb; 3-25 mM succinate buffer system providing a composition pH of 4-6.6; 10-180 mM sodium chloride; 0.4-1.2 mg/mL surfactant; and 5-150 mM amino acid component selected from the group consisting of proline, glycine, lysine, and any combination thereof.
N890. 10-60 mg/mL EGFR/LGR5 BsAb; 3-25 mM succinate buffer system providing a composition pH of 4-6.6; 10-180 mM sodium chloride; 0.4-1.2 mg/mL surfactant; and 5-150 mM amino acid component selected from the group consisting of proline, glycine, lysine, and any combination thereof.
N891. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; and 5-150 mM amino acid component selected from the group consisting of proline, glycine, lysine, and any combination thereof.
N892. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; and 5-150 mM amino acid component selected from the group consisting of proline, glycine, lysine, and any combination thereof.
N893. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 5-150 mM amino acid component selected from the group consisting of proline, glycine, lysine, and any combination thereof.
N894. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 5-150 mM amino acid component selected from the group consisting of proline, glycine, lysine, and any combination thereof.
N895. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 5-150 mM amino acid component selected from the group consisting of proline, glycine, lysine, and any combination thereof.
N896. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 5-150 mM amino acid component selected from the group consisting of proline, glycine, lysine, and any combination thereof.
N897. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; and 5-150 mM amino acid component selected from the group consisting of proline, glycine, lysine, and any combination thereof.
N898. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; and 5-150 mM amino acid component selected from the group consisting of proline, glycine, lysine, and any combination thereof.
N899. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 5-150 mM amino acid component selected from the group consisting of proline, glycine, lysine, and any combination thereof.
N900. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 5-150 mM amino acid component selected from the group consisting of proline, glycine, lysine, and any combination thereof.
N901. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; and 5-150 mM amino acid component selected from the group consisting of proline, glycine, lysine, and any combination thereof.
N902. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; and 5-150 mM amino acid component selected from the group consisting of proline, glycine, lysine, and any combination thereof.
N903. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 5-150 mM amino acid component selected from the group consisting of proline, glycine, lysine, and any combination thereof.
N904. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 5-150 mM amino acid component selected from the group consisting of proline, glycine, lysine, and any combination thereof.
N905. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 5-150 mM amino acid component selected from the group consisting of proline, glycine, lysine, and any combination thereof.
N906. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 5-150 mM amino acid component selected from the group consisting of proline, glycine, lysine, and any combination thereof.
N907. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; and 2-25 mM methionine.
N908. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; and 2-25 mM methionine.
N909. 1-150 mg/mL EGFR/LGR5 BsAb; 3-25 mM acetate buffer system providing a composition pH of 4-5.6; 10-180 mM sodium chloride; 0.4-1.2 mg/mL surfactant; and 2-25 mM methionine.
N910. 10-60 mg/mL EGFR/LGR5 BsAb; 3-25 mM acetate buffer system providing a composition pH of 4-5.6; 10-180 mM sodium chloride; 0.4-1.2 mg/mL surfactant; and 2-25 mM methionine.
N911. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; and 2-25 mM methionine.
N912. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; and 2-25 mM methionine.
N913. 1-150 mg/mL EGFR/LGR5 BsAb; 3-25 mM succinate buffer system providing a composition pH of 4-6.6; 10-180 mM sodium chloride; 0.4-1.2 mg/mL surfactant; and 2-25 mM methionine.
N914. 10-60 mg/mL EGFR/LGR5 BsAb; 3-25 mM succinate buffer system providing a composition pH of 4-6.6; 10-180 mM sodium chloride; 0.4-1.2 mg/mL surfactant; and 2-25 mM methionine.
N915. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; and 2-25 mM methionine.
N916. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; and 2-25 mM methionine.
N917. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 2-25 mM methionine.
N918. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 2-25 mM methionine.
N919. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 2-25 mM methionine.
N920. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 2-25 mM methionine.
N921. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; and 2-25 mM methionine.
N922. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; and 2-25 mM methionine.
N923. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 2-25 mM methionine.
N924. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 2-25 mM methionine.
N925. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; and 2-25 mM methionine.
N926. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; and 2-25 mM methionine.
N927. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 2-25 mM methionine.
N928. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 2-25 mM methionine.
N929. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 2-25 mM methionine.
N930. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 2-25 mM methionine.
N931. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; and 0.01-0.2 mM EDTA.
N932. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; and 0.01-0.2 mM EDTA.
N933. 1-150 mg/mL EGFR/LGR5 BsAb; 3-25 mM acetate buffer system providing a composition pH of 4-5.6; 10-180 mM sodium chloride; 0.4-1.2 mg/mL surfactant; and 0.01-0.2 mM EDTA.
N934. 10-60 mg/mL EGFR/LGR5 BsAb; 3-25 mM acetate buffer system providing a composition pH of 4-5.6; 10-180 mM sodium chloride; 0.4-1.2 mg/mL surfactant; and 0.01-0.2 mM EDTA.
N935. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; and 0.01-0.2 mM EDTA.
N936. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; and 0.01-0.2 mM EDTA.
N937. 1-150 mg/mL EGFR/LGR5 BsAb; 3-25 mM succinate buffer system providing a composition pH of 4-6.6; 10-180 mM sodium chloride; 0.4-1.2 mg/mL surfactant; and 0.01-0.2 mM EDTA.
N938. 10-60 mg/mL EGFR/LGR5 BsAb; 3-25 mM succinate buffer system providing a composition pH of 4-6.6; 10-180 mM sodium chloride; 0.4-1.2 mg/mL surfactant; and 0.01-0.2 mM EDTA.
N939. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; and 0.01-0.2 mM EDTA.
N940. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; and 0.01-0.2 mM EDTA.
N941. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 0.01-0.2 mM EDTA.
N942. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; and 0.01-0.2 mM EDTA.
N943. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 0.01-0.2 mM EDTA.
N944. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; and 0.01-0.2 mM EDTA.
N945. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; and 0.01-0.2 mM EDTA.
N946. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; and 0.01-0.2 mM EDTA.
N947. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 0.01-0.2 mM EDTA.
N948. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; and 0.01-0.2 mM EDTA.
N949. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; and 0.01-0.2 mM EDTA.
N950. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; and 0.01-0.2 mM EDTA.
N951. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 0.01-0.2 mM EDTA.
N952. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; and 0.01-0.2 mM EDTA.
N953. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 0.01-0.2 mM EDTA.
N954. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; and 0.01-0.2 mM EDTA.
N955. 1-150 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; and 2-25 mM methionine.
N956. 10-60 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; and 2-25 mM methionine.
N957. 1-150 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; and 2-25 mM methionine.
N958. 10-60 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; and 2-25 mM methionine.
N959. 1-150 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; and 2-25 mM methionine.
N960. 10-60 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; and 2-25 mM methionine.
N961. 1-150 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; and 2-25 mM methionine.
N962. 10-60 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; and 2-25 mM methionine.
N963. 1-150 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; and 2-25 mM methionine.
N964. 10-60 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; and 2-25 mM methionine.
N965. 1-150 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; and 2-25 mM methionine.
N966. 10-60 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; and 2-25 mM methionine.
N967. 1-150 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; and 0.01-0.2 mM EDTA.
N968. 10-60 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; and 0.01-0.2 mM EDTA.
N969. 1-150 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; and 0.01-0.2 mM EDTA.
N970. 10-60 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; and 0.01-0.2 mM EDTA.
N971. 1-150 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; and 0.01-0.2 mM EDTA.
N972. 10-60 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; and 0.01-0.2 mM EDTA.
N973. 1-150 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; and 0.01-0.2 mM EDTA.
N974. 10-60 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; and 0.01-0.2 mM EDTA.
N975. 1-150 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; and 0.01-0.2 mM EDTA.
N976. 10-60 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; and 0.01-0.2 mM EDTA.
N977. 1-150 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; and 0.01-0.2 mM EDTA.
N978. 10-60 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; and 0.01-0.2 mM EDTA.
N979. 1-150 mg/mL EGFR/LGR5 BsAb; 3-25 mM histidine buffer system providing a composition pH of 5.4-7.1; 10-180 mM sodium chloride; 2-25 mM methionine; and 0.01-0.2 mM EDTA.
N980. 10-60 mg/mL EGFR/LGR5 BsAb; 3-25 mM histidine buffer system providing a composition pH of 5.4-7.1; 10-180 mM sodium chloride; 2-25 mM methionine; and 0.01-0.2 mM EDTA.
N981. 1-150 mg/mL EGFR/LGR5 BsAb; 3-25 mM acetate buffer system providing a composition pH of 4-5.6; 10-180 mM sodium chloride; 2-25 mM methionine; and 0.01-0.2 mM EDTA.
N982. 10-60 mg/mL EGFR/LGR5 BsAb; 3-25 mM acetate buffer system providing a composition pH of 4-5.6; 10-180 mM sodium chloride; 2-25 mM methionine; and 0.01-0.2 mM EDTA.
N983. 1-150 mg/mL EGFR/LGR5 BsAb; 3-25 mM citrate buffer system providing a composition pH of 4.5-7.5; 10-180 mM sodium chloride; 2-25 mM methionine; and 0.01-0.2 mM EDTA.
N984. 10-60 mg/mL EGFR/LGR5 BsAb; 3-25 mM citrate buffer system providing a composition pH of 4.5-7.5; 10-180 mM sodium chloride; 2-25 mM methionine; and 0.01-0.2 mM EDTA.
N985. 1-150 mg/mL EGFR/LGR5 BsAb; 3-25 mM succinate buffer system providing a composition pH of 4-6.6; 10-180 mM sodium chloride; 2-25 mM methionine; and 0.01-0.2 mM EDTA.
N986. 10-60 mg/mL EGFR/LGR5 BsAb; 3-25 mM succinate buffer system providing a composition pH of 4-6.6; 10-180 mM sodium chloride; 2-25 mM methionine; and 0.01-0.2 mM EDTA.
N987. 1-150 mg/mL EGFR/LGR5 BsAb; 3-25 mM phosphate buffer system providing a composition pH of 5.8-8.1; 10-180 mM sodium chloride; 2-25 mM methionine; and 0.01-0.2 mM EDTA.
N988. 10-60 mg/mL EGFR/LGR5 BsAb; 3-25 mM phosphate buffer system providing a composition pH of 5.8-8.1; 10-180 mM sodium chloride; 2-25 mM methionine; and 0.01-0.2 mM EDTA.
N989. 1-150 mg/mL EGFR/LGR5 BsAb; 3-25 mM citrate-phosphate buffer system providing a composition pH of 4-7.1; 10-180 mM sodium chloride; 2-25 mM methionine; and 0.01-0.2 mM EDTA.
N990. 10-60 mg/mL EGFR/LGR5 BsAb; 3-25 mM citrate-phosphate buffer system providing a composition pH of 4-7.1; 10-180 mM sodium chloride; 2-25 mM methionine; and 0.01-0.2 mM EDTA.
N991. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N992. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N993. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N994. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N995. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N996. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N997. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N998. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N999. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1000. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1001. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1002. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1003. 1-150 mg/mL EGFR/LGR5 BsAb; 3-25 mM histidine buffer system providing a composition pH of 5.4-7.1; 150-350 mM sucrose; 0.4-1.2 mg/mL surfactant; and 5-150 mM amino acid component selected from the group consisting of proline, glycine, lysine, and any combination thereof.
N1004. 10-60 mg/mL EGFR/LGR5 BsAb; 3-25 mM histidine buffer system providing a composition pH of 5.4-7.1; 150-350 mM sucrose; 0.4-1.2 mg/mL surfactant; and 5-150 mM amino acid component selected from the group consisting of proline, glycine, lysine, and any combination thereof.
N1005. 1-150 mg/mL EGFR/LGR5 BsAb; 3-25 mM acetate buffer system providing a composition pH of 4-5.6; 150-350 mM sucrose; 0.4-1.2 mg/mL surfactant; and 5-150 mM amino acid component selected from the group consisting of proline, glycine, lysine, and any combination thereof.
N1006. 10-60 mg/mL EGFR/LGR5 BsAb; 3-25 mM acetate buffer system providing a composition pH of 4-5.6; 150-350 mM sucrose; 0.4-1.2 mg/mL surfactant; and 5-150 mM amino acid component selected from the group consisting of proline, glycine, lysine, and any combination thereof.
N1007. 1-150 mg/mL EGFR/LGR5 BsAb; 3-25 mM citrate buffer system providing a composition pH of 4.5-7.5; 150-350 mM sucrose; 0.4-1.2 mg/mL surfactant; and 5-150 mM amino acid component selected from the group consisting of proline, glycine, lysine, and any combination thereof.
N1008. 10-60 mg/mL EGFR/LGR5 BsAb; 3-25 mM citrate buffer system providing a composition pH of 4.5-7.5; 150-350 mM sucrose; 0.4-1.2 mg/mL surfactant; and 5-150 mM amino acid component selected from the group consisting of proline, glycine, lysine, and any combination thereof.
N1009. 1-150 mg/mL EGFR/LGR5 BsAb; 3-25 mM succinate buffer system providing a composition pH of 4-6.6; 150-350 mM sucrose; 0.4-1.2 mg/mL surfactant; and 5-150 mM amino acid component selected from the group consisting of proline, glycine, lysine, and any combination thereof.
N1010. 10-60 mg/mL EGFR/LGR5 BsAb; 3-25 mM succinate buffer system providing a composition pH of 4-6.6; 150-350 mM sucrose; 0.4-1.2 mg/mL surfactant; and 5-150 mM amino acid component selected from the group consisting of proline, glycine, lysine, and any combination thereof.
N1011. 1-150 mg/mL EGFR/LGR5 BsAb; 3-25 mM phosphate buffer system providing a composition pH of 5.8-8.1; 150-350 mM sucrose; 0.4-1.2 mg/mL surfactant; and 5-150 mM amino acid component selected from the group consisting of proline, glycine, lysine, and any combination thereof.
N1012. 10-60 mg/mL EGFR/LGR5 BsAb; 3-25 mM phosphate buffer system providing a composition pH of 5.8-8.1; 150-350 mM sucrose; 0.4-1.2 mg/mL surfactant; and 5-150 mM amino acid component selected from the group consisting of proline, glycine, lysine, and any combination thereof.
N1013. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; and 5-150 mM amino acid component selected from the group consisting of proline, glycine, lysine, and any combination thereof.
N1014. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; and 5-150 mM amino acid component selected from the group consisting of proline, glycine, lysine, and any combination thereof.
N1015. 1-150 mg/mL EGFR/LGR5 BsAb; 3-25 mM histidine buffer system providing a composition pH of 5.4-7.1; 150-350 mM maltose; 0.4-1.2 mg/mL surfactant; and 5-150 mM amino acid component selected from the group consisting of proline, glycine, lysine, and any combination thereof.
N1016. 10-60 mg/mL EGFR/LGR5 BsAb; 3-25 mM histidine buffer system providing a composition pH of 5.4-7.1; 150-350 mM maltose; 0.4-1.2 mg/mL surfactant; and 5-150 mM amino acid component selected from the group consisting of proline, glycine, lysine, and any combination thereof.
N1017. 1-150 mg/mL EGFR/LGR5 BsAb; 3-25 mM acetate buffer system providing a composition pH of 4-5.6; 150-350 mM maltose; 0.4-1.2 mg/mL surfactant; and 5-150 mM amino acid component selected from the group consisting of proline, glycine, lysine, and any combination thereof.
N1018. 10-60 mg/mL EGFR/LGR5 BsAb; 3-25 mM acetate buffer system providing a composition pH of 4-5.6; 150-350 mM maltose; 0.4-1.2 mg/mL surfactant; and 5-150 mM amino acid component selected from the group consisting of proline, glycine, lysine, and any combination thereof.
N1019. 1-150 mg/mL EGFR/LGR5 BsAb; 3-25 mM citrate buffer system providing a composition pH of 4.5-7.5; 150-350 mM maltose; 0.4-1.2 mg/mL surfactant; and 5-150 mM amino acid component selected from the group consisting of proline, glycine, lysine, and any combination thereof.
N1020. 10-60 mg/mL EGFR/LGR5 BsAb; 3-25 mM citrate buffer system providing a composition pH of 4.5-7.5; 150-350 mM maltose; 0.4-1.2 mg/mL surfactant; and 5-150 mM amino acid component selected from the group consisting of proline, glycine, lysine, and any combination thereof.
N1021. 1-150 mg/mL EGFR/LGR5 BsAb; 3-25 mM succinate buffer system providing a composition pH of 4-6.6; 150-350 mM maltose; 0.4-1.2 mg/mL surfactant; and 5-150 mM amino acid component selected from the group consisting of proline, glycine, lysine, and any combination thereof.
N1022. 10-60 mg/mL EGFR/LGR5 BsAb; 3-25 mM succinate buffer system providing a composition pH of 4-6.6; 150-350 mM maltose; 0.4-1.2 mg/mL surfactant; and 5-150 mM amino acid component selected from the group consisting of proline, glycine, lysine, and any combination thereof.
N1023. 1-150 mg/mL EGFR/LGR5 BsAb; 3-25 mM phosphate buffer system providing a composition pH of 5.8-8.1; 150-350 mM maltose; 0.4-1.2 mg/mL surfactant; and 5-150 mM amino acid component selected from the group consisting of proline, glycine, lysine, and any combination thereof.
N1024. 10-60 mg/mL EGFR/LGR5 BsAb; 3-25 mM phosphate buffer system providing a composition pH of 5.8-8.1; 150-350 mM maltose; 0.4-1.2 mg/mL surfactant; and 5-150 mM amino acid component selected from the group consisting of proline, glycine, lysine, and any combination thereof.
N1025. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; and 5-150 mM amino acid component selected from the group consisting of proline, glycine, lysine, and any combination thereof.
N1026. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; and 5-150 mM amino acid component selected from the group consisting of proline, glycine, lysine, and any combination thereof.
N1027. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1028. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1029. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1030. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1031. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1032. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1033. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1034. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1035. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1036. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1037. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1038. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1039. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; and 5-150 mM amino acid component selected from the group consisting of proline, glycine, lysine, and any combination thereof.
N1040. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; and 5-150 mM amino acid component selected from the group consisting of proline, glycine, lysine, and any combination thereof.
N1041. 1-150 mg/mL EGFR/LGR5 BsAb; 3-25 mM acetate buffer system providing a composition pH of 4-5.6; 150-350 mM sucrose; 0.1-1.1 mg/mL polysorbate 80; and 5-150 mM amino acid component selected from the group consisting of proline, glycine, lysine, and any combination thereof.
N1042. 10-60 mg/mL EGFR/LGR5 BsAb; 3-25 mM acetate buffer system providing a composition pH of 4-5.6; 150-350 mM sucrose; 0.1-1.1 mg/mL polysorbate 80; and 5-150 mM amino acid component selected from the group consisting of proline, glycine, lysine, and any combination thereof.
N1043. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; and 5-150 mM amino acid component selected from the group consisting of proline, glycine, lysine, and any combination thereof.
N1044. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; and 5-150 mM amino acid component selected from the group consisting of proline, glycine, lysine, and any combination thereof.
N1045. 1-150 mg/mL EGFR/LGR5 BsAb; 3-25 mM succinate buffer system providing a composition pH of 4-6.6; 150-350 mM sucrose; 0.1-1.1 mg/mL polysorbate 80; and 5-150 mM amino acid component selected from the group consisting of proline, glycine, lysine, and any combination thereof.
N1046. 10-60 mg/mL EGFR/LGR5 BsAb; 3-25 mM succinate buffer system providing a composition pH of 4-6.6; 150-350 mM sucrose; 0.1-1.1 mg/mL polysorbate 80; and 5-150 mM amino acid component selected from the group consisting of proline, glycine, lysine, and any combination thereof.
N1047. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; and 5-150 mM amino acid component selected from the group consisting of proline, glycine, lysine, and any combination thereof.
N1048. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; and 5-150 mM amino acid component selected from the group consisting of proline, glycine, lysine, and any combination thereof.
N1049. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 5-150 mM amino acid component selected from the group consisting of proline, glycine, lysine, and any combination thereof.
N1050. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 5-150 mM amino acid component selected from the group consisting of proline, glycine, lysine, and any combination thereof.
N1051. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; and 5-150 mM amino acid component selected from the group consisting of proline, glycine, lysine, and any combination thereof.
N1052. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; and 5-150 mM amino acid component selected from the group consisting of proline, glycine, lysine, and any combination thereof.
N1053. 1-150 mg/mL EGFR/LGR5 BsAb; 3-25 mM acetate buffer system providing a composition pH of 4-5.6; 150-350 mM maltose; 0.1-1.1 mg/mL polysorbate 80; and 5-150 mM amino acid component selected from the group consisting of proline, glycine, lysine, and any combination thereof.
N1054. 10-60 mg/mL EGFR/LGR5 BsAb; 3-25 mM acetate buffer system providing a composition pH of 4-5.6; 150-350 mM maltose; 0.1-1.1 mg/mL polysorbate 80; and 5-150 mM amino acid component selected from the group consisting of proline, glycine, lysine, and any combination thereof.
N1055. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; and 5-150 mM amino acid component selected from the group consisting of proline, glycine, lysine, and any combination thereof.
N1056. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; and 5-150 mM amino acid component selected from the group consisting of proline, glycine, lysine, and any combination thereof.
N1057. 1-150 mg/mL EGFR/LGR5 BsAb; 3-25 mM succinate buffer system providing a composition pH of 4-6.6; 150-350 mM maltose; 0.1-1.1 mg/mL polysorbate 80; and 5-150 mM amino acid component selected from the group consisting of proline, glycine, lysine, and any combination thereof.
N1058. 10-60 mg/mL EGFR/LGR5 BsAb; 3-25 mM succinate buffer system providing a composition pH of 4-6.6; 150-350 mM maltose; 0.1-1.1 mg/mL polysorbate 80; and 5-150 mM amino acid component selected from the group consisting of proline, glycine, lysine, and any combination thereof.
N1059. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; and 5-150 mM amino acid component selected from the group consisting of proline, glycine, lysine, and any combination thereof.
N1060. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; and 5-150 mM amino acid component selected from the group consisting of proline, glycine, lysine, and any combination thereof.
N1061. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 5-150 mM amino acid component selected from the group consisting of proline, glycine, lysine, and any combination thereof.
N1062. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 5-150 mM amino acid component selected from the group consisting of proline, glycine, lysine, and any combination thereof.
N1063. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 2-25 mM methionine.
N1064. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 2-25 mM methionine.
N1065. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 2-25 mM methionine.
N1066. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 2-25 mM methionine.
N1067. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 2-25 mM methionine.
N1068. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 2-25 mM methionine.
N1069. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 2-25 mM methionine.
N1070. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 2-25 mM methionine.
N1071. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 2-25 mM methionine.
N1072. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 2-25 mM methionine.
N1073. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 2-25 mM methionine.
N1074. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 2-25 mM methionine.
N1075. 1-150 mg/mL EGFR/LGR5 BsAb; 3-25 mM histidine buffer system providing a composition pH of 5.4-7.1; 150-350 mM sucrose; 0.4-1.2 mg/mL surfactant; and 2-25 mM methionine.
N1076. 10-60 mg/mL EGFR/LGR5 BsAb; 3-25 mM histidine buffer system providing a composition pH of 5.4-7.1; 150-350 mM sucrose; 0.4-1.2 mg/mL surfactant; and 2-25 mM methionine.
N1077. 1-150 mg/mL EGFR/LGR5 BsAb; 3-25 mM acetate buffer system providing a composition pH of 4-5.6; 150-350 mM sucrose; 0.4-1.2 mg/mL surfactant; and 2-25 mM methionine.
N1078. 10-60 mg/mL EGFR/LGR5 BsAb; 3-25 mM acetate buffer system providing a composition pH of 4-5.6; 150-350 mM sucrose; 0.4-1.2 mg/mL surfactant; and 2-25 mM methionine.
N1079. 1-150 mg/mL EGFR/LGR5 BsAb; 3-25 mM citrate buffer system providing a composition pH of 4.5-7.5; 150-350 mM sucrose; 0.4-1.2 mg/mL surfactant; and 2-25 mM methionine.
N1080. 10-60 mg/mL EGFR/LGR5 BsAb; 3-25 mM citrate buffer system providing a composition pH of 4.5-7.5; 150-350 mM sucrose; 0.4-1.2 mg/mL surfactant; and 2-25 mM methionine.
N1081. 1-150 mg/mL EGFR/LGR5 BsAb; 3-25 mM succinate buffer system providing a composition pH of 4-6.6; 150-350 mM sucrose; 0.4-1.2 mg/mL surfactant; and 2-25 mM methionine.
N1082. 10-60 mg/mL EGFR/LGR5 BsAb; 3-25 mM succinate buffer system providing a composition pH of 4-6.6; 150-350 mM sucrose; 0.4-1.2 mg/mL surfactant; and 2-25 mM methionine.
N1083. 1-150 mg/mL EGFR/LGR5 BsAb; 3-25 mM phosphate buffer system providing a composition pH of 5.8-8.1; 150-350 mM sucrose; 0.4-1.2 mg/mL surfactant; and 2-25 mM methionine.
N1084. 10-60 mg/mL EGFR/LGR5 BsAb; 3-25 mM phosphate buffer system providing a composition pH of 5.8-8.1; 150-350 mM sucrose; 0.4-1.2 mg/mL surfactant; and 2-25 mM methionine.
N1085. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; and 2-25 mM methionine.
N1086. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; and 2-25 mM methionine.
N1087. 1-150 mg/mL EGFR/LGR5 BsAb; 3-25 mM histidine buffer system providing a composition pH of 5.4-7.1; 150-350 mM maltose; 0.4-1.2 mg/mL surfactant; and 2-25 mM methionine.
N1088. 10-60 mg/mL EGFR/LGR5 BsAb; 3-25 mM histidine buffer system providing a composition pH of 5.4-7.1; 150-350 mM maltose; 0.4-1.2 mg/mL surfactant; and 2-25 mM methionine.
N1089. 1-150 mg/mL EGFR/LGR5 BsAb; 3-25 mM acetate buffer system providing a composition pH of 4-5.6; 150-350 mM maltose; 0.4-1.2 mg/mL surfactant; and 2-25 mM methionine.
N1090. 10-60 mg/mL EGFR/LGR5 BsAb; 3-25 mM acetate buffer system providing a composition pH of 4-5.6; 150-350 mM maltose; 0.4-1.2 mg/mL surfactant; and 2-25 mM methionine.
N1091. 1-150 mg/mL EGFR/LGR5 BsAb; 3-25 mM citrate buffer system providing a composition pH of 4.5-7.5; 150-350 mM maltose; 0.4-1.2 mg/mL surfactant; and 2-25 mM methionine.
N1092. 10-60 mg/mL EGFR/LGR5 BsAb; 3-25 mM citrate buffer system providing a composition pH of 4.5-7.5; 150-350 mM maltose; 0.4-1.2 mg/mL surfactant; and 2-25 mM methionine.
N1093. 1-150 mg/mL EGFR/LGR5 BsAb; 3-25 mM succinate buffer system providing a composition pH of 4-6.6; 150-350 mM maltose; 0.4-1.2 mg/mL surfactant; and 2-25 mM methionine.
N1094. 10-60 mg/mL EGFR/LGR5 BsAb; 3-25 mM succinate buffer system providing a composition pH of 4-6.6; 150-350 mM maltose; 0.4-1.2 mg/mL surfactant; and 2-25 mM methionine.
N1095. 1-150 mg/mL EGFR/LGR5 BsAb; 3-25 mM phosphate buffer system providing a composition pH of 5.8-8.1; 150-350 mM maltose; 0.4-1.2 mg/mL surfactant; and 2-25 mM methionine.
N1096. 10-60 mg/mL EGFR/LGR5 BsAb; 3-25 mM phosphate buffer system providing a composition pH of 5.8-8.1; 150-350 mM maltose; 0.4-1.2 mg/mL surfactant; and 2-25 mM methionine.
N1097. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; and 2-25 mM methionine.
N1098. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; and 2-25 mM methionine.
N1099. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 2-25 mM methionine.
N1100. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 2-25 mM methionine.
N1101. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 2-25 mM methionine.
N1102. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 2-25 mM methionine.
N1103. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 2-25 mM methionine.
N1104. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 2-25 mM methionine.
N1105. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 2-25 mM methionine.
N1106. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 2-25 mM methionine.
N1107. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 2-25 mM methionine.
N1108. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 2-25 mM methionine.
N1109. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 2-25 mM methionine.
N1110. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 2-25 mM methionine.
N1111. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; and 2-25 mM methionine.
N1112. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; and 2-25 mM methionine.
N1113. 1-150 mg/mL EGFR/LGR5 BsAb; 3-25 mM acetate buffer system providing a composition pH of 4-5.6; 150-350 mM sucrose; 0.1-1.1 mg/mL polysorbate 80; and 2-25 mM methionine.
N1114. 10-60 mg/mL EGFR/LGR5 BsAb; 3-25 mM acetate buffer system providing a composition pH of 4-5.6; 150-350 mM sucrose; 0.1-1.1 mg/mL polysorbate 80; and 2-25 mM methionine.
N1115. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; and 2-25 mM methionine.
N1116. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; and 2-25 mM methionine.
N1117. 1-150 mg/mL EGFR/LGR5 BsAb; 3-25 mM succinate buffer system providing a composition pH of 4-6.6; 150-350 mM sucrose; 0.1-1.1 mg/mL polysorbate 80; and 2-25 mM methionine.
N1118. 10-60 mg/mL EGFR/LGR5 BsAb; 3-25 mM succinate buffer system providing a composition pH of 4-6.6; 150-350 mM sucrose; 0.1-1.1 mg/mL polysorbate 80; and 2-25 mM methionine.
N1119. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; and 2-25 mM methionine.
N1120. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; and 2-25 mM methionine.
N1121. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 2-25 mM methionine.
N1122. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 2-25 mM methionine.
N1123. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; and 2-25 mM methionine.
N1124. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; and 2-25 mM methionine.
N1125. 1-150 mg/mL EGFR/LGR5 BsAb; 3-25 mM acetate buffer system providing a composition pH of 4-5.6; 150-350 mM maltose; 0.1-1.1 mg/mL polysorbate 80; and 2-25 mM methionine.
N1126. 10-60 mg/mL EGFR/LGR5 BsAb; 3-25 mM acetate buffer system providing a composition pH of 4-5.6; 150-350 mM maltose; 0.1-1.1 mg/mL polysorbate 80; and 2-25 mM methionine.
N1127. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; and 2-25 mM methionine.
N1128. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; and 2-25 mM methionine.
N1129. 1-150 mg/mL EGFR/LGR5 BsAb; 3-25 mM succinate buffer system providing a composition pH of 4-6.6; 150-350 mM maltose; 0.1-1.1 mg/mL polysorbate 80; and 2-25 mM methionine.
N1130. 10-60 mg/mL EGFR/LGR5 BsAb; 3-25 mM succinate buffer system providing a composition pH of 4-6.6; 150-350 mM maltose; 0.1-1.1 mg/mL polysorbate 80; and 2-25 mM methionine.
N1131. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; and 2-25 mM methionine.
N1132. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; and 2-25 mM methionine.
N1133. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 2-25 mM methionine.
N1134. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 2-25 mM methionine.
N1135. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 0.01-0.2 mM EDTA.
N1136. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; and 0.01-0.2 mM EDTA.
N1137. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 0.01-0.2 mM EDTA.
N1138. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; and 0.01-0.2 mM EDTA.
N1139. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 0.01-0.2 mM EDTA.
N1140. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; and 0.01-0.2 mM EDTA.
N1141. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 0.01-0.2 mM EDTA.
N1142. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; and 0.01-0.2 mM EDTA.
N1143. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 0.01-0.2 mM EDTA.
N1144. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; and 0.01-0.2 mM EDTA.
N1145. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 0.01-0.2 mM EDTA.
N1146. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; and 0.01-0.2 mM EDTA.
N1147. 1-150 mg/mL EGFR/LGR5 BsAb; 3-25 mM histidine buffer system providing a composition pH of 5.4-7.1; 150-350 mM sucrose; 0.4-1.2 mg/mL surfactant; and 0.01-0.2 mM EDTA.
N1148. 10-60 mg/mL EGFR/LGR5 BsAb; 3-25 mM histidine buffer system providing a composition pH of 5.4-7.1; 150-350 mM sucrose; 0.4-1.2 mg/mL surfactant; and 0.01-0.2 mM EDTA.
N1149. 1-150 mg/mL EGFR/LGR5 BsAb; 3-25 mM acetate buffer system providing a composition pH of 4-5.6; 150-350 mM sucrose; 0.4-1.2 mg/mL surfactant; and 0.01-0.2 mM EDTA.
N1150. 10-60 mg/mL EGFR/LGR5 BsAb; 3-25 mM acetate buffer system providing a composition pH of 4-5.6; 150-350 mM sucrose; 0.4-1.2 mg/mL surfactant; and 0.01-0.2 mM EDTA.
N1151. 1-150 mg/mL EGFR/LGR5 BsAb; 3-25 mM citrate buffer system providing a composition pH of 4.5-7.5; 150-350 mM sucrose; 0.4-1.2 mg/mL surfactant; and 0.01-0.2 mM EDTA.
N1152. 10-60 mg/mL EGFR/LGR5 BsAb; 3-25 mM citrate buffer system providing a composition pH of 4.5-7.5; 150-350 mM sucrose; 0.4-1.2 mg/mL surfactant; and 0.01-0.2 mM EDTA.
N1153. 1-150 mg/mL EGFR/LGR5 BsAb; 3-25 mM succinate buffer system providing a composition pH of 4-6.6; 150-350 mM sucrose; 0.4-1.2 mg/mL surfactant; and 0.01-0.2 mM EDTA.
N1154. 10-60 mg/mL EGFR/LGR5 BsAb; 3-25 mM succinate buffer system providing a composition pH of 4-6.6; 150-350 mM sucrose; 0.4-1.2 mg/mL surfactant; and 0.01-0.2 mM EDTA.
N1155. 1-150 mg/mL EGFR/LGR5 BsAb; 3-25 mM phosphate buffer system providing a composition pH of 5.8-8.1; 150-350 mM sucrose; 0.4-1.2 mg/mL surfactant; and 0.01-0.2 mM EDTA.
N1156. 10-60 mg/mL EGFR/LGR5 BsAb; 3-25 mM phosphate buffer system providing a composition pH of 5.8-8.1; 150-350 mM sucrose; 0.4-1.2 mg/mL surfactant; and 0.01-0.2 mM EDTA.
N1157. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; and 0.01-0.2 mM EDTA.
N1158. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; and 0.01-0.2 mM EDTA.
N1159. 1-150 mg/mL EGFR/LGR5 BsAb; 3-25 mM histidine buffer system providing a composition pH of 5.4-7.1; 150-350 mM maltose; 0.4-1.2 mg/mL surfactant; and 0.01-0.2 mM EDTA.
N1160. 10-60 mg/mL EGFR/LGR5 BsAb; 3-25 mM histidine buffer system providing a composition pH of 5.4-7.1; 150-350 mM maltose; 0.4-1.2 mg/mL surfactant; and 0.01-0.2 mM EDTA.
N1161. 1-150 mg/mL EGFR/LGR5 BsAb; 3-25 mM acetate buffer system providing a composition pH of 4-5.6; 150-350 mM maltose; 0.4-1.2 mg/mL surfactant; and 0.01-0.2 mM EDTA.
N1162. 10-60 mg/mL EGFR/LGR5 BsAb; 3-25 mM acetate buffer system providing a composition pH of 4-5.6; 150-350 mM maltose; 0.4-1.2 mg/mL surfactant; and 0.01-0.2 mM EDTA.
N1163. 1-150 mg/mL EGFR/LGR5 BsAb; 3-25 mM citrate buffer system providing a composition pH of 4.5-7.5; 150-350 mM maltose; 0.4-1.2 mg/mL surfactant; and 0.01-0.2 mM EDTA.
N1164. 10-60 mg/mL EGFR/LGR5 BsAb; 3-25 mM citrate buffer system providing a composition pH of 4.5-7.5; 150-350 mM maltose; 0.4-1.2 mg/mL surfactant; and 0.01-0.2 mM EDTA.
N1165. 1-150 mg/mL EGFR/LGR5 BsAb; 3-25 mM succinate buffer system providing a composition pH of 4-6.6; 150-350 mM maltose; 0.4-1.2 mg/mL surfactant; and 0.01-0.2 mM EDTA.
N1166. 10-60 mg/mL EGFR/LGR5 BsAb; 3-25 mM succinate buffer system providing a composition pH of 4-6.6; 150-350 mM maltose; 0.4-1.2 mg/mL surfactant; and 0.01-0.2 mM EDTA.
N1167. 1-150 mg/mL EGFR/LGR5 BsAb; 3-25 mM phosphate buffer system providing a composition pH of 5.8-8.1; 150-350 mM maltose; 0.4-1.2 mg/mL surfactant; and 0.01-0.2 mM EDTA.
N1168. 10-60 mg/mL EGFR/LGR5 BsAb; 3-25 mM phosphate buffer system providing a composition pH of 5.8-8.1; 150-350 mM maltose; 0.4-1.2 mg/mL surfactant; and 0.01-0.2 mM EDTA.
N1169. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; and 0.01-0.2 mM EDTA.
N1170. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; and 0.01-0.2 mM EDTA.
N1171. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 0.01-0.2 mM EDTA.
N1172. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; and 0.01-0.2 mM EDTA.
N1173. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 0.01-0.2 mM EDTA.
N1174. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; and 0.01-0.2 mM EDTA.
N1175. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 0.01-0.2 mM EDTA.
N1176. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; and 0.01-0.2 mM EDTA.
N1177. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 0.01-0.2 mM EDTA.
N1178. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; and 0.01-0.2 mM EDTA.
N1179. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 0.01-0.2 mM EDTA.
N1180. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; and 0.01-0.2 mM EDTA.
N1181. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 0.01-0.2 mM EDTA.
N1182. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; and 0.01-0.2 mM EDTA.
N1183. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; and 0.01-0.2 mM EDTA.
N1184. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; and 0.01-0.2 mM EDTA.
N1185. 1-150 mg/mL EGFR/LGR5 BsAb; 3-25 mM acetate buffer system providing a composition pH of 4-5.6; 150-350 mM sucrose; 0.1-1.1 mg/mL polysorbate 80; and 0.01-0.2 mM EDTA.
N1186. 10-60 mg/mL EGFR/LGR5 BsAb; 3-25 mM acetate buffer system providing a composition pH of 4-5.6; 150-350 mM sucrose; 0.1-1.1 mg/mL polysorbate 80; and 0.01-0.2 mM EDTA.
N1187. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; and 0.01-0.2 mM EDTA.
N1188. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; and 0.01-0.2 mM EDTA.
N1189. 1-150 mg/mL EGFR/LGR5 BsAb; 3-25 mM succinate buffer system providing a composition pH of 4-6.6; 150-350 mM sucrose; 0.1-1.1 mg/mL polysorbate 80; and 0.01-0.2 mM EDTA.
N1190. 10-60 mg/mL EGFR/LGR5 BsAb; 3-25 mM succinate buffer system providing a composition pH of 4-6.6; 150-350 mM sucrose; 0.1-1.1 mg/mL polysorbate 80; and 0.01-0.2 mM EDTA.
N1191. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; and 0.01-0.2 mM EDTA.
N1192. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; and 0.01-0.2 mM EDTA.
N1193. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 0.01-0.2 mM EDTA.
N1194. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; and 0.01-0.2 mM EDTA.
N1195. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; and 0.01-0.2 mM EDTA.
N1196. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; and 0.01-0.2 mM EDTA.
N1197. 1-150 mg/mL EGFR/LGR5 BsAb; 3-25 mM acetate buffer system providing a composition pH of 4-5.6; 150-350 mM maltose; 0.1-1.1 mg/mL polysorbate 80; and 0.01-0.2 mM EDTA.
N1198. 10-60 mg/mL EGFR/LGR5 BsAb; 3-25 mM acetate buffer system providing a composition pH of 4-5.6; 150-350 mM maltose; 0.1-1.1 mg/mL polysorbate 80; and 0.01-0.2 mM EDTA.
N1199. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; and 0.01-0.2 mM EDTA.
N1200. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; and 0.01-0.2 mM EDTA.
N1201. 1-150 mg/mL EGFR/LGR5 BsAb; 3-25 mM succinate buffer system providing a composition pH of 4-6.6; 150-350 mM maltose; 0.1-1.1 mg/mL polysorbate 80; and 0.01-0.2 mM EDTA.
N1202. 10-60 mg/mL EGFR/LGR5 BsAb; 3-25 mM succinate buffer system providing a composition pH of 4-6.6; 150-350 mM maltose; 0.1-1.1 mg/mL polysorbate 80; and 0.01-0.2 mM EDTA.
N1203. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; and 0.01-0.2 mM EDTA.
N1204. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; and 0.01-0.2 mM EDTA.
N1205. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 0.01-0.2 mM EDTA.
N1206. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; and 0.01-0.2 mM EDTA.
N1207. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1208. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1209. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1210. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1211. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1212. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1213. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1214. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1215. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1216. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1217. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1218. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1219. 1-150 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; and 2-25 mM methionine.
N1220. 10-60 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; and 2-25 mM methionine.
N1221. 1-150 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; and 2-25 mM methionine.
N1222. 10-60 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; and 2-25 mM methionine.
N1223. 1-150 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; and 2-25 mM methionine.
N1224. 10-60 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; and 2-25 mM methionine.
N1225. 1-150 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; and 2-25 mM methionine.
N1226. 10-60 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; and 2-25 mM methionine.
N1227. 1-150 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; and 2-25 mM methionine.
N1228. 10-60 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; and 2-25 mM methionine.
N1229. 1-150 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; and 2-25 mM methionine.
N1230. 10-60 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; and 2-25 mM methionine.
N1231. 1-150 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; and 2-25 mM methionine.
N1232. 10-60 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; and 2-25 mM methionine.
N1233. 1-150 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; and 2-25 mM methionine.
N1234. 10-60 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; and 2-25 mM methionine.
N1235. 1-150 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; and 2-25 mM methionine.
N1236. 10-60 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; and 2-25 mM methionine.
N1237. 1-150 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; and 2-25 mM methionine.
N1238. 10-60 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; and 2-25 mM methionine.
N1239. 1-150 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; and 2-25 mM methionine.
N1240. 10-60 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; and 2-25 mM methionine.
N1241. 1-150 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; and 2-25 mM methionine.
N1242. 10-60 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; and 2-25 mM methionine.
N1243. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1244. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1245. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1246. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1247. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1248. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1249. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1250. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1251. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1252. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1253. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1254. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1255. 1-150 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; and 0.01-0.2 mM EDTA.
N1256. 10-60 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; and 0.01-0.2 mM EDTA.
N1257. 1-150 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; and 0.01-0.2 mM EDTA.
N1258. 10-60 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; and 0.01-0.2 mM EDTA.
N1259. 1-150 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; and 0.01-0.2 mM EDTA.
N1260. 10-60 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; and 0.01-0.2 mM EDTA.
N1261. 1-150 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; and 0.01-0.2 mM EDTA.
N1262. 10-60 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; and 0.01-0.2 mM EDTA.
N1263. 1-150 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; and 0.01-0.2 mM EDTA.
N1264. 10-60 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; and 0.01-0.2 mM EDTA.
N1265. 1-150 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; and 0.01-0.2 mM EDTA.
N1266. 10-60 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; and 0.01-0.2 mM EDTA.
N1267. 1-150 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; and 0.01-0.2 mM EDTA.
N1268. 10-60 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; and 0.01-0.2 mM EDTA.
N1269. 1-150 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; and 0.01-0.2 mM EDTA.
N1270. 10-60 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; and 0.01-0.2 mM EDTA.
N1271. 1-150 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; and 0.01-0.2 mM EDTA.
N1272. 10-60 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; and 0.01-0.2 mM EDTA.
N1273. 1-150 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; and 0.01-0.2 mM EDTA.
N1274. 10-60 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; and 0.01-0.2 mM EDTA.
N1275. 1-150 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; and 0.01-0.2 mM EDTA.
N1276. 10-60 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; and 0.01-0.2 mM EDTA.
N1277. 1-150 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; and 0.01-0.2 mM EDTA.
N1278. 10-60 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; and 0.01-0.2 mM EDTA.
N1279. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 2-25 mM methionine; and 0.01-0.2 mM EDTA.
N1280. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 2-25 mM methionine; and 0.01-0.2 mM EDTA.
N1281. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 2-25 mM methionine; and 0.01-0.2 mM EDTA.
N1282. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 2-25 mM methionine; and 0.01-0.2 mM EDTA.
N1283. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 2-25 mM methionine; and 0.01-0.2 mM EDTA.
N1284. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 2-25 mM methionine; and 0.01-0.2 mM EDTA.
N1285. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 2-25 mM methionine; and 0.01-0.2 mM EDTA.
N1286. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 2-25 mM methionine; and 0.01-0.2 mM EDTA.
N1287. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 2-25 mM methionine; and 0.01-0.2 mM EDTA.
N1288. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 2-25 mM methionine; and 0.01-0.2 mM EDTA.
N1289. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 2-25 mM methionine; and 0.01-0.2 mM EDTA.
N1290. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 2-25 mM methionine; and 0.01-0.2 mM EDTA.
N1291. 1-150 mg/mL EGFR/LGR5 BsAb; 3-25 mM histidine buffer system providing a composition pH of 5.4-7.1; 150-350 mM sucrose; 2-25 mM methionine; and 0.01-0.2 mM EDTA.
N1292. 10-60 mg/mL EGFR/LGR5 BsAb; 3-25 mM histidine buffer system providing a composition pH of 5.4-7.1; 150-350 mM sucrose; 2-25 mM methionine; and 0.01-0.2 mM EDTA.
N1293. 1-150 mg/mL EGFR/LGR5 BsAb; 3-25 mM acetate buffer system providing a composition pH of 4-5.6; 150-350 mM sucrose; 2-25 mM methionine; and 0.01-0.2 mM EDTA.
N1294. 10-60 mg/mL EGFR/LGR5 BsAb; 3-25 mM acetate buffer system providing a composition pH of 4-5.6; 150-350 mM sucrose; 2-25 mM methionine; and 0.01-0.2 mM EDTA.
N1295. 1-150 mg/mL EGFR/LGR5 BsAb; 3-25 mM citrate buffer system providing a composition pH of 4.5-7.5; 150-350 mM sucrose; 2-25 mM methionine; and 0.01-0.2 mM EDTA.
N1296. 10-60 mg/mL EGFR/LGR5 BsAb; 3-25 mM citrate buffer system providing a composition pH of 4.5-7.5; 150-350 mM sucrose; 2-25 mM methionine; and 0.01-0.2 mM EDTA.
N1297. 1-150 mg/mL EGFR/LGR5 BsAb; 3-25 mM succinate buffer system providing a composition pH of 4-6.6; 150-350 mM sucrose; 2-25 mM methionine; and 0.01-0.2 mM EDTA.
N1298. 10-60 mg/mL EGFR/LGR5 BsAb; 3-25 mM succinate buffer system providing a composition pH of 4-6.6; 150-350 mM sucrose; 2-25 mM methionine; and 0.01-0.2 mM EDTA.
N1299. 1-150 mg/mL EGFR/LGR5 BsAb; 3-25 mM phosphate buffer system providing a composition pH of 5.8-8.1; 150-350 mM sucrose; 2-25 mM methionine; and 0.01-0.2 mM EDTA.
N1300. 10-60 mg/mL EGFR/LGR5 BsAb; 3-25 mM phosphate buffer system providing a composition pH of 5.8-8.1; 150-350 mM sucrose; 2-25 mM methionine; and 0.01-0.2 mM EDTA.
N1301. 1-150 mg/mL EGFR/LGR5 BsAb; 3-25 mM citrate-phosphate buffer system providing a composition pH of 4-7.1; 150-350 mM sucrose; 2-25 mM methionine; and 0.01-0.2 mM EDTA.
N1302. 10-60 mg/mL EGFR/LGR5 BsAb; 3-25 mM citrate-phosphate buffer system providing a composition pH of 4-7.1; 150-350 mM sucrose; 2-25 mM methionine; and 0.01-0.2 mM EDTA.
N1303. 1-150 mg/mL EGFR/LGR5 BsAb; 3-25 mM histidine buffer system providing a composition pH of 5.4-7.1; 150-350 mM maltose; 2-25 mM methionine; and 0.01-0.2 mM EDTA.
N1304. 10-60 mg/mL EGFR/LGR5 BsAb; 3-25 mM histidine buffer system providing a composition pH of 5.4-7.1; 150-350 mM maltose; 2-25 mM methionine; and 0.01-0.2 mM EDTA.
N1305. 1-150 mg/mL EGFR/LGR5 BsAb; 3-25 mM acetate buffer system providing a composition pH of 4-5.6; 150-350 mM maltose; 2-25 mM methionine; and 0.01-0.2 mM EDTA.
N1306. 10-60 mg/mL EGFR/LGR5 BsAb; 3-25 mM acetate buffer system providing a composition pH of 4-5.6; 150-350 mM maltose; 2-25 mM methionine; and 0.01-0.2 mM EDTA.
N1307. 1-150 mg/mL EGFR/LGR5 BsAb; 3-25 mM citrate buffer system providing a composition pH of 4.5-7.5; 150-350 mM maltose; 2-25 mM methionine; and 0.01-0.2 mM EDTA.
N1308. 10-60 mg/mL EGFR/LGR5 BsAb; 3-25 mM citrate buffer system providing a composition pH of 4.5-7.5; 150-350 mM maltose; 2-25 mM methionine; and 0.01-0.2 mM EDTA.
N1309. 1-150 mg/mL EGFR/LGR5 BsAb; 3-25 mM succinate buffer system providing a composition pH of 4-6.6; 150-350 mM maltose; 2-25 mM methionine; and 0.01-0.2 mM EDTA.
N1310. 10-60 mg/mL EGFR/LGR5 BsAb; 3-25 mM succinate buffer system providing a composition pH of 4-6.6; 150-350 mM maltose; 2-25 mM methionine; and 0.01-0.2 mM EDTA.
N1311. 1-150 mg/mL EGFR/LGR5 BsAb; 3-25 mM phosphate buffer system providing a composition pH of 5.8-8.1; 150-350 mM maltose; 2-25 mM methionine; and 0.01-0.2 mM EDTA.
N1312. 10-60 mg/mL EGFR/LGR5 BsAb; 3-25 mM phosphate buffer system providing a composition pH of 5.8-8.1; 150-350 mM maltose; 2-25 mM methionine; and 0.01-0.2 mM EDTA.
N1313. 1-150 mg/mL EGFR/LGR5 BsAb; 3-25 mM citrate-phosphate buffer system providing a composition pH of 4-7.1; 150-350 mM maltose; 2-25 mM methionine; and 0.01-0.2 mM EDTA.
N1314. 10-60 mg/mL EGFR/LGR5 BsAb; 3-25 mM citrate-phosphate buffer system providing a composition pH of 4-7.1; 150-350 mM maltose; 2-25 mM methionine; and 0.01-0.2 mM EDTA.
N1315. 1-150 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; and 2-25 mM methionine.
N1316. 10-60 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; and 2-25 mM methionine.
N1317. 1-150 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; and 2-25 mM methionine.
N1318. 10-60 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; and 2-25 mM methionine.
N1319. 1-150 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; and 2-25 mM methionine.
N1320. 10-60 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; and 2-25 mM methionine.
N1321. 1-150 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; and 2-25 mM methionine.
N1322. 10-60 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; and 2-25 mM methionine.
N1323. 1-150 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; and 2-25 mM methionine.
N1324. 10-60 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; and 2-25 mM methionine.
N1325. 1-150 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; and 2-25 mM methionine.
N1326. 10-60 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; and 2-25 mM methionine.
N1327. 1-150 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; and 2-25 mM methionine.
N1328. 10-60 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; and 2-25 mM methionine.
N1329. 1-150 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; and 2-25 mM methionine.
N1330. 10-60 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; and 2-25 mM methionine.
N1331. 1-150 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; and 2-25 mM methionine.
N1332. 10-60 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; and 2-25 mM methionine.
N1333. 1-150 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; and 2-25 mM methionine.
N1334. 10-60 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; and 2-25 mM methionine.
N1335. 1-150 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; and 2-25 mM methionine.
N1336. 10-60 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; and 2-25 mM methionine.
N1337. 1-150 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; and 2-25 mM methionine.
N1338. 10-60 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; and 2-25 mM methionine.
N1339. 1-150 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; and 0.01-0.2 mM EDTA.
N1340. 10-60 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; and 0.01-0.2 mM EDTA.
N1341. 1-150 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; and 0.01-0.2 mM EDTA.
N1342. 10-60 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; and 0.01-0.2 mM EDTA.
N1343. 1-150 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; and 0.01-0.2 mM EDTA.
N1344. 10-60 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; and 0.01-0.2 mM EDTA.
N1345. 1-150 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; and 0.01-0.2 mM EDTA.
N1346. 10-60 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; and 0.01-0.2 mM EDTA.
N1347. 1-150 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; and 0.01-0.2 mM EDTA.
N1348. 10-60 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; and 0.01-0.2 mM EDTA.
N1349. 1-150 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; and 0.01-0.2 mM EDTA.
N1350. 10-60 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; and 0.01-0.2 mM EDTA.
N1351. 1-150 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; and 0.01-0.2 mM EDTA.
N1352. 10-60 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; and 0.01-0.2 mM EDTA.
N1353. 1-150 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; and 0.01-0.2 mM EDTA.
N1354. 10-60 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; and 0.01-0.2 mM EDTA.
N1355. 1-150 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; and 0.01-0.2 mM EDTA.
N1356. 10-60 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; and 0.01-0.2 mM EDTA.
N1357. 1-150 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; and 0.01-0.2 mM EDTA.
N1358. 10-60 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; and 0.01-0.2 mM EDTA.
N1359. 1-150 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; and 0.01-0.2 mM EDTA.
N1360. 10-60 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; and 0.01-0.2 mM EDTA.
N1361. 1-150 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; and 0.01-0.2 mM EDTA.
N1362. 10-60 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; and 0.01-0.2 mM EDTA.
N1363. 1-150 mg/mL EGFR/LGR5 BsAb; 3-25 mM histidine buffer system providing a composition pH of 5.4-7.1; 0.4-1.2 mg/mL surfactant; 2-25 mM methionine; and 0.01-0.2 mM EDTA.
N1364. 10-60 mg/mL EGFR/LGR5 BsAb; 3-25 mM histidine buffer system providing a composition pH of 5.4-7.1; 0.4-1.2 mg/mL surfactant; 2-25 mM methionine; and 0.01-0.2 mM EDTA.
N1365. 1-150 mg/mL EGFR/LGR5 BsAb; 3-25 mM acetate buffer system providing a composition pH of 4-5.6; 0.4-1.2 mg/mL surfactant; 2-25 mM methionine; and 0.01-0.2 mM EDTA.
N1366. 10-60 mg/mL EGFR/LGR5 BsAb; 3-25 mM acetate buffer system providing a composition pH of 4-5.6; 0.4-1.2 mg/mL surfactant; 2-25 mM methionine; and 0.01-0.2 mM EDTA.
N1367. 1-150 mg/mL EGFR/LGR5 BsAb; 3-25 mM citrate buffer system providing a composition pH of 4.5-7.5; 0.4-1.2 mg/mL surfactant; 2-25 mM methionine; and 0.01-0.2 mM EDTA.
N1368. 10-60 mg/mL EGFR/LGR5 BsAb; 3-25 mM citrate buffer system providing a composition pH of 4.5-7.5; 0.4-1.2 mg/mL surfactant; 2-25 mM methionine; and 0.01-0.2 mM EDTA.
N1369. 1-150 mg/mL EGFR/LGR5 BsAb; 3-25 mM succinate buffer system providing a composition pH of 4-6.6; 0.4-1.2 mg/mL surfactant; 2-25 mM methionine; and 0.01-0.2 mM EDTA.
N1370. 10-60 mg/mL EGFR/LGR5 BsAb; 3-25 mM succinate buffer system providing a composition pH of 4-6.6; 0.4-1.2 mg/mL surfactant; 2-25 mM methionine; and 0.01-0.2 mM EDTA.
N1371. 1-150 mg/mL EGFR/LGR5 BsAb; 3-25 mM phosphate buffer system providing a composition pH of 5.8-8.1; 0.4-1.2 mg/mL surfactant; 2-25 mM methionine; and 0.01-0.2 mM EDTA.
N1372. 10-60 mg/mL EGFR/LGR5 BsAb; 3-25 mM phosphate buffer system providing a composition pH of 5.8-8.1; 0.4-1.2 mg/mL surfactant; 2-25 mM methionine; and 0.01-0.2 mM EDTA.
N1373. 1-150 mg/mL EGFR/LGR5 BsAb; 3-25 mM citrate-phosphate buffer system providing a composition pH of 4-7.1; 0.4-1.2 mg/mL surfactant; 2-25 mM methionine; and 0.01-0.2 mM EDTA.
N1374. 10-60 mg/mL EGFR/LGR5 BsAb; 3-25 mM citrate-phosphate buffer system providing a composition pH of 4-7.1; 0.4-1.2 mg/mL surfactant; 2-25 mM methionine; and 0.01-0.2 mM EDTA.
N1375. 1-150 mg/mL EGFR/LGR5 BsAb; 3-25 mM histidine buffer system providing a composition pH of 5.4-7.1; 0.1-1.1 mg/mL polysorbate 80; 2-25 mM methionine; and 0.01-0.2 mM EDTA.
N1376. 10-60 mg/mL EGFR/LGR5 BsAb; 3-25 mM histidine buffer system providing a composition pH of 5.4-7.1; 0.1-1.1 mg/mL polysorbate 80; 2-25 mM methionine; and 0.01-0.2 mM EDTA.
N1377. 1-150 mg/mL EGFR/LGR5 BsAb; 3-25 mM acetate buffer system providing a composition pH of 4-5.6; 0.1-1.1 mg/mL polysorbate 80; 2-25 mM methionine; and 0.01-0.2 mM EDTA.
N1378. 10-60 mg/mL EGFR/LGR5 BsAb; 3-25 mM acetate buffer system providing a composition pH of 4-5.6; 0.1-1.1 mg/mL polysorbate 80; 2-25 mM methionine; and 0.01-0.2 mM EDTA.
N1379. 1-150 mg/mL EGFR/LGR5 BsAb; 3-25 mM citrate buffer system providing a composition pH of 4.5-7.5; 0.1-1.1 mg/mL polysorbate 80; 2-25 mM methionine; and 0.01-0.2 mM EDTA.
N1380. 10-60 mg/mL EGFR/LGR5 BsAb; 3-25 mM citrate buffer system providing a composition pH of 4.5-7.5; 0.1-1.1 mg/mL polysorbate 80; 2-25 mM methionine; and 0.01-0.2 mM EDTA.
N1381. 1-150 mg/mL EGFR/LGR5 BsAb; 3-25 mM succinate buffer system providing a composition pH of 4-6.6; 0.1-1.1 mg/mL polysorbate 80; 2-25 mM methionine; and 0.01-0.2 mM EDTA.
N1382. 10-60 mg/mL EGFR/LGR5 BsAb; 3-25 mM succinate buffer system providing a composition pH of 4-6.6; 0.1-1.1 mg/mL polysorbate 80; 2-25 mM methionine; and 0.01-0.2 mM EDTA.
N1383. 1-150 mg/mL EGFR/LGR5 BsAb; 3-25 mM phosphate buffer system providing a composition pH of 5.8-8.1; 0.1-1.1 mg/mL polysorbate 80; 2-25 mM methionine; and 0.01-0.2 mM EDTA.
N1384. 10-60 mg/mL EGFR/LGR5 BsAb; 3-25 mM phosphate buffer system providing a composition pH of 5.8-8.1; 0.1-1.1 mg/mL polysorbate 80; 2-25 mM methionine; and 0.01-0.2 mM EDTA.
N1385. 1-150 mg/mL EGFR/LGR5 BsAb; 3-25 mM citrate-phosphate buffer system providing a composition pH of 4-7.1; 0.1-1.1 mg/mL polysorbate 80; 2-25 mM methionine; and 0.01-0.2 mM EDTA.
N1386. 10-60 mg/mL EGFR/LGR5 BsAb; 3-25 mM citrate-phosphate buffer system providing a composition pH of 4-7.1; 0.1-1.1 mg/mL polysorbate 80; 2-25 mM methionine; and 0.01-0.2 mM EDTA.
N1387. 1-150 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; 2-25 mM methionine; and 0.01-0.2 mM EDTA.
N1388. 10-60 mg/mL EGFR/LGR5 BsAb; 3-25 mM histidine buffer system providing a composition pH of 5.4-7.1; 5-150 mM amino acid component selected from the group consisting of proline, glycine, lysine, and any combination thereof; 2-25 mM methionine; and 0.01-0.2 mM EDTA.
N1389. 1-150 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; 2-25 mM methionine; and 0.01-0.2 mM EDTA.
N1390. 10-60 mg/mL EGFR/LGR5 BsAb; 3-25 mM acetate buffer system providing a composition pH of 4-5.6; 5-150 mM amino acid component selected from the group consisting of proline, glycine, lysine, and any combination thereof; 2-25 mM methionine; and 0.01-0.2 mM EDTA.
N1391. 1-150 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; 2-25 mM methionine; and 0.01-0.2 mM EDTA.
N1392. 10-60 mg/mL EGFR/LGR5 BsAb; 3-25 mM citrate buffer system providing a composition pH of 4.5-7.5; 5-150 mM amino acid component selected from the group consisting of proline, glycine, lysine, and any combination thereof; 2-25 mM methionine; and 0.01-0.2 mM EDTA.
N1393. 1-150 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; 2-25 mM methionine; and 0.01-0.2 mM EDTA.
N1394. 10-60 mg/mL EGFR/LGR5 BsAb; 3-25 mM succinate buffer system providing a composition pH of 4-6.6; 5-150 mM amino acid component selected from the group consisting of proline, glycine, lysine, and any combination thereof; 2-25 mM methionine; and 0.01-0.2 mM EDTA.
N1395. 1-150 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; 2-25 mM methionine; and 0.01-0.2 mM EDTA.
N1396. 10-60 mg/mL EGFR/LGR5 BsAb; 3-25 mM phosphate buffer system providing a composition pH of 5.8-8.1; 5-150 mM amino acid component selected from the group consisting of proline, glycine, lysine, and any combination thereof; 2-25 mM methionine; and 0.01-0.2 mM EDTA.
N1397. 1-150 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; 2-25 mM methionine; and 0.01-0.2 mM EDTA.
N1398. 10-60 mg/mL EGFR/LGR5 BsAb; 3-25 mM citrate-phosphate buffer system providing a composition pH of 4-7.1; 5-150 mM amino acid component selected from the group consisting of proline, glycine, lysine, and any combination thereof; 2-25 mM methionine; and 0.01-0.2 mM EDTA.
N1399. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1400. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1401. 1-150 mg/mL EGFR/LGR5 BsAb; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1402. 10-60 mg/mL EGFR/LGR5 BsAb; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1403. 1-150 mg/mL EGFR/LGR5 BsAb; 10-180 mM sodium chloride; 150-350 mM maltose; 0.4-1.2 mg/mL surfactant; and 5-150 mM amino acid component selected from the group consisting of proline, glycine, lysine, and any combination thereof.
N1404. 10-60 mg/mL EGFR/LGR5 BsAb; 10-180 mM sodium chloride; 150-350 mM maltose; 0.4-1.2 mg/mL surfactant; and 5-150 mM amino acid component selected from the group consisting of proline, glycine, lysine, and any combination thereof.
N1405. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1406. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1407. 1-150 mg/mL EGFR/LGR5 BsAb; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1408. 10-60 mg/mL EGFR/LGR5 BsAb; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1409. 1-150 mg/mL EGFR/LGR5 BsAb; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1410. 10-60 mg/mL EGFR/LGR5 BsAb; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1411. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1412. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1413. 1-150 mg/mL EGFR/LGR5 BsAb; 10-180 mM sodium chloride; 150-350 mM sucrose; 0.4-1.2 mg/mL surfactant; and 2-25 mM methionine.
N1414. 10-60 mg/mL EGFR/LGR5 BsAb; 10-180 mM sodium chloride; 150-350 mM sucrose; 0.4-1.2 mg/mL surfactant; and 2-25 mM methionine.
N1415. 1-150 mg/mL EGFR/LGR5 BsAb; 10-180 mM sodium chloride; 150-350 mM maltose; 0.4-1.2 mg/mL surfactant; and 2-25 mM methionine.
N1416. 10-60 mg/mL EGFR/LGR5 BsAb; 10-180 mM sodium chloride; 150-350 mM maltose; 0.4-1.2 mg/mL surfactant; and 2-25 mM methionine.
N1417. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1418. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1419. 1-150 mg/mL EGFR/LGR5 BsAb; 10-180 mM sodium chloride; 150-350 mM sucrose; 0.1-1.1 mg/mL polysorbate 80; and 2-25 mM methionine.
N1420. 10-60 mg/mL EGFR/LGR5 BsAb; 10-180 mM sodium chloride; 150-350 mM sucrose; 0.1-1.1 mg/mL polysorbate 80; and 2-25 mM methionine.
N1421. 1-150 mg/mL EGFR/LGR5 BsAb; 10-180 mM sodium chloride; 150-350 mM maltose; 0.1-1.1 mg/mL polysorbate 80; and 2-25 mM methionine.
N1422. 10-60 mg/mL EGFR/LGR5 BsAb; 10-180 mM sodium chloride; 150-350 mM maltose; 0.1-1.1 mg/mL polysorbate 80; and 2-25 mM methionine.
N1423. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1424. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1425. 1-150 mg/mL EGFR/LGR5 BsAb; 10-180 mM sodium chloride; 150-350 mM sucrose; 0.4-1.2 mg/mL surfactant; and 0.01-0.2 mM EDTA.
N1426. 10-60 mg/mL EGFR/LGR5 BsAb; 10-180 mM sodium chloride; 150-350 mM sucrose; 0.4-1.2 mg/mL surfactant; and 0.01-0.2 mM EDTA.
N1427. 1-150 mg/mL EGFR/LGR5 BsAb; 10-180 mM sodium chloride; 150-350 mM maltose; 0.4-1.2 mg/mL surfactant; and 0.01-0.2 mM EDTA.
N1428. 10-60 mg/mL EGFR/LGR5 BsAb; 10-180 mM sodium chloride; 150-350 mM maltose; 0.4-1.2 mg/mL surfactant; and 0.01-0.2 mM EDTA.
N1429. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1430. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1431. 1-150 mg/mL EGFR/LGR5 BsAb; 10-180 mM sodium chloride; 150-350 mM sucrose; 0.1-1.1 mg/mL polysorbate 80; and 0.01-0.2 mM EDTA.
N1432. 10-60 mg/mL EGFR/LGR5 BsAb; 10-180 mM sodium chloride; 150-350 mM sucrose; 0.1-1.1 mg/mL polysorbate 80; and 0.01-0.2 mM EDTA.
N1433. 1-150 mg/mL EGFR/LGR5 BsAb; 10-180 mM sodium chloride; 150-350 mM maltose; 0.1-1.1 mg/mL polysorbate 80; and 0.01-0.2 mM EDTA.
N1434. 10-60 mg/mL EGFR/LGR5 BsAb; 10-180 mM sodium chloride; 150-350 mM maltose; 0.1-1.1 mg/mL polysorbate 80; and 0.01-0.2 mM EDTA.
N1435. 1-150 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1436. 10-60 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1437. 1-150 mg/mL EGFR/LGR5 BsAb; 10-180 mM sodium chloride; 150-350 mM sucrose; 5-150 mM amino acid component selected from the group consisting of proline, glycine, lysine, and any combination thereof; and 2-25 mM methionine.
N1438. 10-60 mg/mL EGFR/LGR5 BsAb; 10-180 mM sodium chloride; 150-350 mM sucrose; 5-150 mM amino acid component selected from the group consisting of proline, glycine, lysine, and any combination thereof; and 2-25 mM methionine.
N1439. 1-150 mg/mL EGFR/LGR5 BsAb; 10-180 mM sodium chloride; 150-350 mM maltose; 5-150 mM amino acid component selected from the group consisting of proline, glycine, lysine, and any combination thereof; and 2-25 mM methionine.
N1440. 10-60 mg/mL EGFR/LGR5 BsAb; 10-180 mM sodium chloride; 150-350 mM maltose; 5-150 mM amino acid component selected from the group consisting of proline, glycine, lysine, and any combination thereof; and 2-25 mM methionine.
N1441. 1-150 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1442. 10-60 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1443. 1-150 mg/mL EGFR/LGR5 BsAb; 10-180 mM sodium chloride; 150-350 mM sucrose; 5-150 mM amino acid component selected from the group consisting of proline, glycine, lysine, and any combination thereof; and 0.01-0.2 mM EDTA.
N1444. 10-60 mg/mL EGFR/LGR5 BsAb; 10-180 mM sodium chloride; 150-350 mM sucrose; 5-150 mM amino acid component selected from the group consisting of proline, glycine, lysine, and any combination thereof; and 0.01-0.2 mM EDTA.
N1445. 1-150 mg/mL EGFR/LGR5 BsAb; 10-180 mM sodium chloride; 150-350 mM maltose; 5-150 mM amino acid component selected from the group consisting of proline, glycine, lysine, and any combination thereof; and 0.01-0.2 mM EDTA.
N1446. 10-60 mg/mL EGFR/LGR5 BsAb; 10-180 mM sodium chloride; 150-350 mM maltose; 5-150 mM amino acid component selected from the group consisting of proline, glycine, lysine, and any combination thereof; and 0.01-0.2 mM EDTA.
N1447. 1-150 mg/mL EGFR/LGR5 BsAb; 10-180 mM sodium chloride; 100-400 mM sugar component selected from the group consisting of sucrose, mannitol, sorbitol, and maltose; 2-25 mM methionine; and 0.01-0.2 mM EDTA.
N1448. 10-60 mg/mL EGFR/LGR5 BsAb; 10-180 mM sodium chloride; 100-400 mM sugar component selected from the group consisting of sucrose, mannitol, sorbitol, and maltose; 2-25 mM methionine; and 0.01-0.2 mM EDTA.
N1449. 1-150 mg/mL EGFR/LGR5 BsAb; 10-180 mM sodium chloride; 150-350 mM sucrose; 2-25 mM methionine; and 0.01-0.2 mM EDTA.
N1450. 10-60 mg/mL EGFR/LGR5 BsAb; 10-180 mM sodium chloride; 150-350 mM sucrose; 2-25 mM methionine; and 0.01-0.2 mM EDTA.
N1451. 1-150 mg/mL EGFR/LGR5 BsAb; 10-180 mM sodium chloride; 150-350 mM maltose; 2-25 mM methionine; and 0.01-0.2 mM EDTA.
N1452. 10-60 mg/mL EGFR/LGR5 BsAb; 10-180 mM sodium chloride; 150-350 mM maltose; 2-25 mM methionine; and 0.01-0.2 mM EDTA.
N1453. 1-150 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; and 2-25 mM methionine.
N1454. 10-60 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; and 2-25 mM methionine.
N1455. 1-150 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1456. 10-60 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1457. 1-150 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1458. 10-60 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1459. 1-150 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1460. 10-60 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1461. 1-150 mg/mL EGFR/LGR5 BsAb; 10-180 mM sodium chloride; 0.4-1.2 mg/mL surfactant; 2-25 mM methionine; and 0.01-0.2 mM EDTA.
N1462. 10-60 mg/mL EGFR/LGR5 BsAb; 10-180 mM sodium chloride; 0.4-1.2 mg/mL surfactant; 2-25 mM methionine; and 0.01-0.2 mM EDTA.
N1463. 1-150 mg/mL EGFR/LGR5 BsAb; 10-180 mM sodium chloride; 0.1-1.1 mg/mL polysorbate 80; 2-25 mM methionine; and 0.01-0.2 mM EDTA.
N1464. 10-60 mg/mL EGFR/LGR5 BsAb; 10-180 mM sodium chloride; 0.1-1.1 mg/mL polysorbate 80; 2-25 mM methionine; and 0.01-0.2 mM EDTA.
N1465. 1-150 mg/mL EGFR/LGR5 BsAb; 10-180 mM sodium chloride; 5-150 mM amino acid component selected from the group consisting of proline, glycine, lysine, and any combination thereof; 2-25 mM methionine; and 0.01-0.2 mM EDTA.
N1466. 10-60 mg/mL EGFR/LGR5 BsAb; 10-180 mM sodium chloride; 5-150 mM amino acid component selected from the group consisting of proline, glycine, lysine, and any combination thereof; 2-25 mM methionine; and 0.01-0.2 mM EDTA.
N1467. 1-150 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1468. 10-60 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1469. 1-150 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; and 2-25 mM methionine.
N1470. 10-60 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; and 2-25 mM methionine.
N1471. 1-150 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; and 2-25 mM methionine.
N1472. 10-60 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; and 2-25 mM methionine.
N1473. 1-150 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1474. 10-60 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1475. 1-150 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1476. 10-60 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1477. 1-150 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; and 2-25 mM methionine.
N1478. 10-60 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; and 2-25 mM methionine.
N1479. 1-150 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1480. 10-60 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1481. 1-150 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N1482. 10-60 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N1483. 1-150 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N1484. 10-60 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N1485. 1-150 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N1486. 10-60 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N1487. 1-150 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N1488. 10-60 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N1489. 1-150 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N1490. 10-60 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N1491. 1-150 mg/mL EGFR/LGR5 BsAb; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N1492. 10-60 mg/mL EGFR/LGR5 BsAb; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N1493. 1-150 mg/mL EGFR/LGR5 BsAb; 150-350 mM sucrose; 0.4-1.2 mg/mL surfactant; 2-25 mM methionine; and 0.01-0.2 mM EDTA.
N1494. 10-60 mg/mL EGFR/LGR5 BsAb; 150-350 mM sucrose; 0.4-1.2 mg/mL surfactant; 2-25 mM methionine; and 0.01-0.2 mM EDTA.
N1495. 1-150 mg/mL EGFR/LGR5 BsAb; 150-350 mM maltose; 0.4-1.2 mg/mL surfactant; 2-25 mM methionine; and 0.01-0.2 mM EDTA.
N1496. 10-60 mg/mL EGFR/LGR5 BsAb; 150-350 mM maltose; 0.4-1.2 mg/mL surfactant; 2-25 mM methionine; and 0.01-0.2 mM EDTA.
N1497. 1-150 mg/mL EGFR/LGR5 BsAb; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N1498. 10-60 mg/mL EGFR/LGR5 BsAb; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N1499. 1-150 mg/mL EGFR/LGR5 BsAb; 150-350 mM sucrose; 0.1-1.1 mg/mL polysorbate 80; 2-25 mM methionine; and 0.01-0.2 mM EDTA.
N1500. 10-60 mg/mL EGFR/LGR5 BsAb; 150-350 mM sucrose; 0.1-1.1 mg/mL polysorbate 80; 2-25 mM methionine; and 0.01-0.2 mM EDTA.
N1501. 1-150 mg/mL EGFR/LGR5 BsAb; 150-350 mM maltose; 0.1-1.1 mg/mL polysorbate 80; 2-25 mM methionine; and 0.01-0.2 mM EDTA.
N1502. 10-60 mg/mL EGFR/LGR5 BsAb; 150-350 mM maltose; 0.1-1.1 mg/mL polysorbate 80; 2-25 mM methionine; and 0.01-0.2 mM EDTA.
N1503. 1-150 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N1504. 10-60 mg/mL EGFR/LGR5 BsAb; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N1505. 1-150 mg/mL EGFR/LGR5 BsAb; 150-350 mM sucrose; 5-150 mM amino acid component selected from the group consisting of proline, glycine, lysine, and any combination thereof; 2-25 mM methionine; and 0.01-0.2 mM EDTA.
N1506. 10-60 mg/mL EGFR/LGR5 BsAb; 150-350 mM sucrose; 5-150 mM amino acid component selected from the group consisting of proline, glycine, lysine, and any combination thereof; 2-25 mM methionine; and 0.01-0.2 mM EDTA.
N1507. 1-150 mg/mL EGFR/LGR5 BsAb; 150-350 mM maltose; 5-150 mM amino acid component selected from the group consisting of proline, glycine, lysine, and any combination thereof; 2-25 mM methionine; and 0.01-0.2 mM EDTA.
N1508. 10-60 mg/mL EGFR/LGR5 BsAb; 150-350 mM maltose; 5-150 mM amino acid component selected from the group consisting of proline, glycine, lysine, and any combination thereof; 2-25 mM methionine; and 0.01-0.2 mM EDTA.
N1509. 1-150 mg/mL EGFR/LGR5 BsAb; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N1510. 10-60 mg/mL EGFR/LGR5 BsAb; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N1511. 1-150 mg/mL EGFR/LGR5 BsAb; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N1512. 10-60 mg/mL EGFR/LGR5 BsAb; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N1513. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1514. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1515. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1516. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1517. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1518. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1519. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1520. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1521. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1522. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1523. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1524. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 5-150 mM amino acid component selected from the group consisting of proline, glycine, lysine, and any combination thereof.
N1525. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1526. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1527. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1528. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1529. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1530. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1531. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1532. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1533. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1534. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1535. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1536. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 5-150 mM amino acid component selected from the group consisting of proline, glycine, lysine, and any combination thereof.
N1537. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1538. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1539. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 5-150 mM amino acid component selected from the group consisting of proline, glycine, lysine, and any combination thereof.
N1540. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 5-150 mM amino acid component selected from the group consisting of proline, glycine, lysine, and any combination thereof.
N1541. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 5-150 mM amino acid component selected from the group consisting of proline, glycine, lysine, and any combination thereof.
N1542. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 5-150 mM amino acid component selected from the group consisting of proline, glycine, lysine, and any combination thereof.
N1543. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 5-150 mM amino acid component selected from the group consisting of proline, glycine, lysine, and any combination thereof.
N1544. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 5-150 mM amino acid component selected from the group consisting of proline, glycine, lysine, and any combination thereof.
N1545. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1546. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1547. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1548. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 5-150 mM amino acid component selected from the group consisting of proline, glycine, lysine, and any combination thereof.
N1549. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1550. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1551. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1552. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1553. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1554. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1555. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1556. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1557. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1558. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1559. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1560. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 5-150 mM amino acid component selected from the group consisting of proline, glycine, lysine, and any combination thereof.
N1561. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1562. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1563. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1564. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1565. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1566. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1567. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1568. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1569. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1570. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1571. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1572. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 5-150 mM amino acid component selected from the group consisting of proline, glycine, lysine, and any combination thereof.
N1573. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1574. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1575. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1576. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1577. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1578. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1579. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1580. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1581. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1582. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1583. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1584. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 5-150 mM amino acid component selected from the group consisting of proline, glycine, lysine, and any combination thereof.
N1585. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1586. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1587. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1588. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1589. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1590. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1591. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1592. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1593. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1594. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1595. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1596. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 2-25 mM methionine.
N1597. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 2-25 mM methionine.
N1598. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 2-25 mM methionine.
N1599. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 2-25 mM methionine.
N1600. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 2-25 mM methionine.
N1601. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 2-25 mM methionine.
N1602. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 2-25 mM methionine.
N1603. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 2-25 mM methionine.
N1604. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 2-25 mM methionine.
N1605. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 2-25 mM methionine.
N1606. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 2-25 mM methionine.
N1607. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 2-25 mM methionine.
N1608. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 2-25 mM methionine.
N1609. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 2-25 mM methionine.
N1610. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 2-25 mM methionine.
N1611. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 2-25 mM methionine.
N1612. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 2-25 mM methionine.
N1613. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 2-25 mM methionine.
N1614. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 2-25 mM methionine.
N1615. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 2-25 mM methionine.
N1616. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 2-25 mM methionine.
N1617. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 2-25 mM methionine.
N1618. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 2-25 mM methionine.
N1619. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 2-25 mM methionine.
N1620. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 2-25 mM methionine.
N1621. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1622. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1623. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1624. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1625. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1626. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1627. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1628. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1629. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1630. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1631. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1632. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 2-25 mM methionine.
N1633. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 2-25 mM methionine.
N1634. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 2-25 mM methionine.
N1635. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 2-25 mM methionine.
N1636. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 2-25 mM methionine.
N1637. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 2-25 mM methionine.
N1638. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 2-25 mM methionine.
N1639. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 2-25 mM methionine.
N1640. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 2-25 mM methionine.
N1641. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 2-25 mM methionine.
N1642. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 2-25 mM methionine.
N1643. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 2-25 mM methionine.
N1644. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 2-25 mM methionine.
N1645. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 2-25 mM methionine.
N1646. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 2-25 mM methionine.
N1647. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 2-25 mM methionine.
N1648. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 2-25 mM methionine.
N1649. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 2-25 mM methionine.
N1650. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 2-25 mM methionine.
N1651. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 2-25 mM methionine.
N1652. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 2-25 mM methionine.
N1653. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 2-25 mM methionine.
N1654. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 2-25 mM methionine.
N1655. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 2-25 mM methionine.
N1656. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 2-25 mM methionine.
N1657. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1658. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1659. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1660. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1661. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1662. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1663. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1664. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1665. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1666. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1667. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1668. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; and 0.01-0.2 mM EDTA.
N1669. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 0.01-0.2 mM EDTA.
N1670. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; and 0.01-0.2 mM EDTA.
N1671. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 0.01-0.2 mM EDTA.
N1672. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; and 0.01-0.2 mM EDTA.
N1673. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 0.01-0.2 mM EDTA.
N1674. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; and 0.01-0.2 mM EDTA.
N1675. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 0.01-0.2 mM EDTA.
N1676. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; and 0.01-0.2 mM EDTA.
N1677. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 0.01-0.2 mM EDTA.
N1678. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; and 0.01-0.2 mM EDTA.
N1679. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 0.01-0.2 mM EDTA.
N1680. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; and 0.01-0.2 mM EDTA.
N1681. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 0.01-0.2 mM EDTA.
N1682. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; and 0.01-0.2 mM EDTA.
N1683. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 0.01-0.2 mM EDTA.
N1684. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; and 0.01-0.2 mM EDTA.
N1685. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 0.01-0.2 mM EDTA.
N1686. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; and 0.01-0.2 mM EDTA.
N1687. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 0.01-0.2 mM EDTA.
N1688. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; and 0.01-0.2 mM EDTA.
N1689. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 0.01-0.2 mM EDTA.
N1690. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; and 0.01-0.2 mM EDTA.
N1691. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 0.01-0.2 mM EDTA.
N1692. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; and 0.01-0.2 mM EDTA.
N1693. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1694. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1695. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1696. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1697. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1698. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1699. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1700. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1701. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1702. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1703. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1704. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; and 0.01-0.2 mM EDTA.
N1705. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 0.01-0.2 mM EDTA.
N1706. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; and 0.01-0.2 mM EDTA.
N1707. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 0.01-0.2 mM EDTA.
N1708. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; and 0.01-0.2 mM EDTA.
N1709. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 0.01-0.2 mM EDTA.
N1710. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; and 0.01-0.2 mM EDTA.
N1711. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 0.01-0.2 mM EDTA.
N1712. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; and 0.01-0.2 mM EDTA.
N1713. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 0.01-0.2 mM EDTA.
N1714. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; and 0.01-0.2 mM EDTA.
N1715. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 0.01-0.2 mM EDTA.
N1716. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; and 0.01-0.2 mM EDTA.
N1717. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 0.01-0.2 mM EDTA.
N1718. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; and 0.01-0.2 mM EDTA.
N1719. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 0.01-0.2 mM EDTA.
N1720. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; and 0.01-0.2 mM EDTA.
N1721. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 0.01-0.2 mM EDTA.
N1722. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; and 0.01-0.2 mM EDTA.
N1723. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 0.01-0.2 mM EDTA.
N1724. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; and 0.01-0.2 mM EDTA.
N1725. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 0.01-0.2 mM EDTA.
N1726. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; and 0.01-0.2 mM EDTA.
N1727. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 0.01-0.2 mM EDTA.
N1728. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; and 0.01-0.2 mM EDTA.
N1729. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1730. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1731. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1732. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1733. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1734. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1735. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1736. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1737. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1738. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1739. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1740. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 2-25 mM methionine.
N1741. 1-150 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; and 2-25 mM methionine.
N1742. 10-60 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; and 2-25 mM methionine.
N1743. 1-150 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; and 2-25 mM methionine.
N1744. 10-60 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; and 2-25 mM methionine.
N1745. 1-150 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; and 2-25 mM methionine.
N1746. 10-60 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; and 2-25 mM methionine.
N1747. 1-150 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; and 2-25 mM methionine.
N1748. 10-60 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; and 2-25 mM methionine.
N1749. 1-150 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; and 2-25 mM methionine.
N1750. 10-60 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; and 2-25 mM methionine.
N1751. 1-150 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; and 2-25 mM methionine.
N1752. 10-60 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; and 2-25 mM methionine.
N1753. 1-150 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; and 2-25 mM methionine.
N1754. 10-60 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; and 2-25 mM methionine.
N1755. 1-150 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; and 2-25 mM methionine.
N1756. 10-60 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; and 2-25 mM methionine.
N1757. 1-150 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; and 2-25 mM methionine.
N1758. 10-60 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; and 2-25 mM methionine.
N1759. 1-150 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; and 2-25 mM methionine.
N1760. 10-60 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; and 2-25 mM methionine.
N1761. 1-150 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; and 2-25 mM methionine.
N1762. 10-60 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; and 2-25 mM methionine.
N1763. 1-150 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; and 2-25 mM methionine.
N1764. 10-60 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; and 2-25 mM methionine.
N1765. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1766. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1767. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1768. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1769. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1770. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1771. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1772. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1773. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1774. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1775. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1776. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; and 0.01-0.2 mM EDTA.
N1777. 1-150 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; and 0.01-0.2 mM EDTA.
N1778. 10-60 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; and 0.01-0.2 mM EDTA.
N1779. 1-150 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; and 0.01-0.2 mM EDTA.
N1780. 10-60 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; and 0.01-0.2 mM EDTA.
N1781. 1-150 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; and 0.01-0.2 mM EDTA.
N1782. 10-60 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; and 0.01-0.2 mM EDTA.
N1783. 1-150 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; and 0.01-0.2 mM EDTA.
N1784. 10-60 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; and 0.01-0.2 mM EDTA.
N1785. 1-150 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; and 0.01-0.2 mM EDTA.
N1786. 10-60 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; and 0.01-0.2 mM EDTA.
N1787. 1-150 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; and 0.01-0.2 mM EDTA.
N1788. 10-60 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; and 0.01-0.2 mM EDTA.
N1789. 1-150 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; and 0.01-0.2 mM EDTA.
N1790. 10-60 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; and 0.01-0.2 mM EDTA.
N1791. 1-150 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; and 0.01-0.2 mM EDTA.
N1792. 10-60 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; and 0.01-0.2 mM EDTA.
N1793. 1-150 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; and 0.01-0.2 mM EDTA.
N1794. 10-60 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; and 0.01-0.2 mM EDTA.
N1795. 1-150 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; and 0.01-0.2 mM EDTA.
N1796. 10-60 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; and 0.01-0.2 mM EDTA.
N1797. 1-150 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; and 0.01-0.2 mM EDTA.
N1798. 10-60 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; and 0.01-0.2 mM EDTA.
N1799. 1-150 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; and 0.01-0.2 mM EDTA.
N1800. 10-60 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; and 0.01-0.2 mM EDTA.
N1801. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 2-25 mM methionine; and 0.01-0.2 mM EDTA.
N1802. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 2-25 mM methionine; and 0.01-0.2 mM EDTA.
N1803. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 2-25 mM methionine; and 0.01-0.2 mM EDTA.
N1804. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 2-25 mM methionine; and 0.01-0.2 mM EDTA.
N1805. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 2-25 mM methionine; and 0.01-0.2 mM EDTA.
N1806. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 2-25 mM methionine; and 0.01-0.2 mM EDTA.
N1807. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 2-25 mM methionine; and 0.01-0.2 mM EDTA.
N1808. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 2-25 mM methionine; and 0.01-0.2 mM EDTA.
N1809. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 2-25 mM methionine; and 0.01-0.2 mM EDTA.
N1810. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 2-25 mM methionine; and 0.01-0.2 mM EDTA.
N1811. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 2-25 mM methionine; and 0.01-0.2 mM EDTA.
N1812. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 2-25 mM methionine; and 0.01-0.2 mM EDTA.
N1813. 1-150 mg/mL EGFR/LGR5 BsAb; 3-25 mM histidine buffer system providing a composition pH of 5.4-7.1; 10-180 mM sodium chloride; 150-350 mM sucrose; 2-25 mM methionine; and 0.01-0.2 mM EDTA.
N1814. 10-60 mg/mL EGFR/LGR5 BsAb; 3-25 mM histidine buffer system providing a composition pH of 5.4-7.1; 10-180 mM sodium chloride; 150-350 mM sucrose; 2-25 mM methionine; and 0.01-0.2 mM EDTA.
N1815. 1-150 mg/mL EGFR/LGR5 BsAb; 3-25 mM acetate buffer system providing a composition pH of 4-5.6; 10-180 mM sodium chloride; 150-350 mM sucrose; 2-25 mM methionine; and 0.01-0.2 mM EDTA.
N1816. 10-60 mg/mL EGFR/LGR5 BsAb; 3-25 mM acetate buffer system providing a composition pH of 4-5.6; 10-180 mM sodium chloride; 150-350 mM sucrose; 2-25 mM methionine; and 0.01-0.2 mM EDTA.
N1817. 1-150 mg/mL EGFR/LGR5 BsAb; 3-25 mM citrate buffer system providing a composition pH of 4.5-7.5; 10-180 mM sodium chloride; 150-350 mM sucrose; 2-25 mM methionine; and 0.01-0.2 mM EDTA.
N1818. 10-60 mg/mL EGFR/LGR5 BsAb; 3-25 mM citrate buffer system providing a composition pH of 4.5-7.5; 10-180 mM sodium chloride; 150-350 mM sucrose; 2-25 mM methionine; and 0.01-0.2 mM EDTA.
N1819. 1-150 mg/mL EGFR/LGR5 BsAb; 3-25 mM succinate buffer system providing a composition pH of 4-6.6; 10-180 mM sodium chloride; 150-350 mM sucrose; 2-25 mM methionine; and 0.01-0.2 mM EDTA.
N1820. 10-60 mg/mL EGFR/LGR5 BsAb; 3-25 mM succinate buffer system providing a composition pH of 4-6.6; 10-180 mM sodium chloride; 150-350 mM sucrose; 2-25 mM methionine; and 0.01-0.2 mM EDTA.
N1821. 1-150 mg/mL EGFR/LGR5 BsAb; 3-25 mM phosphate buffer system providing a composition pH of 5.8-8.1; 10-180 mM sodium chloride; 150-350 mM sucrose; 2-25 mM methionine; and 0.01-0.2 mM EDTA.
N1822. 10-60 mg/mL EGFR/LGR5 BsAb; 3-25 mM phosphate buffer system providing a composition pH of 5.8-8.1; 10-180 mM sodium chloride; 150-350 mM sucrose; 2-25 mM methionine; and 0.01-0.2 mM EDTA.
N1823. 1-150 mg/mL EGFR/LGR5 BsAb; 3-25 mM citrate-phosphate buffer system providing a composition pH of 4-7.1; 10-180 mM sodium chloride; 150-350 mM sucrose; 2-25 mM methionine; and 0.01-0.2 mM EDTA.
N1824. 10-60 mg/mL EGFR/LGR5 BsAb; 3-25 mM citrate-phosphate buffer system providing a composition pH of 4-7.1; 10-180 mM sodium chloride; 150-350 mM sucrose; 2-25 mM methionine; and 0.01-0.2 mM EDTA.
N1825. 1-150 mg/mL EGFR/LGR5 BsAb; 3-25 mM histidine buffer system providing a composition pH of 5.4-7.1; 10-180 mM sodium chloride; 150-350 mM maltose; 2-25 mM methionine; and 0.01-0.2 mM EDTA.
N1826. 10-60 mg/mL EGFR/LGR5 BsAb; 3-25 mM histidine buffer system providing a composition pH of 5.4-7.1; 10-180 mM sodium chloride; 150-350 mM maltose; 2-25 mM methionine; and 0.01-0.2 mM EDTA.
N1827. 1-150 mg/mL EGFR/LGR5 BsAb; 3-25 mM acetate buffer system providing a composition pH of 4-5.6; 10-180 mM sodium chloride; 150-350 mM maltose; 2-25 mM methionine; and 0.01-0.2 mM EDTA.
N1828. 10-60 mg/mL EGFR/LGR5 BsAb; 3-25 mM acetate buffer system providing a composition pH of 4-5.6; 10-180 mM sodium chloride; 150-350 mM maltose; 2-25 mM methionine; and 0.01-0.2 mM EDTA.
N1829. 1-150 mg/mL EGFR/LGR5 BsAb; 3-25 mM citrate buffer system providing a composition pH of 4.5-7.5; 10-180 mM sodium chloride; 150-350 mM maltose; 2-25 mM methionine; and 0.01-0.2 mM EDTA.
N1830. 10-60 mg/mL EGFR/LGR5 BsAb; 3-25 mM citrate buffer system providing a composition pH of 4.5-7.5; 10-180 mM sodium chloride; 150-350 mM maltose; 2-25 mM methionine; and 0.01-0.2 mM EDTA.
N1831. 1-150 mg/mL EGFR/LGR5 BsAb; 3-25 mM succinate buffer system providing a composition pH of 4-6.6; 10-180 mM sodium chloride; 150-350 mM maltose; 2-25 mM methionine; and 0.01-0.2 mM EDTA.
N1832. 10-60 mg/mL EGFR/LGR5 BsAb; 3-25 mM succinate buffer system providing a composition pH of 4-6.6; 10-180 mM sodium chloride; 150-350 mM maltose; 2-25 mM methionine; and 0.01-0.2 mM EDTA.
N1833. 1-150 mg/mL EGFR/LGR5 BsAb; 3-25 mM phosphate buffer system providing a composition pH of 5.8-8.1; 10-180 mM sodium chloride; 150-350 mM maltose; 2-25 mM methionine; and 0.01-0.2 mM EDTA.
N1834. 10-60 mg/mL EGFR/LGR5 BsAb; 3-25 mM phosphate buffer system providing a composition pH of 5.8-8.1; 10-180 mM sodium chloride; 150-350 mM maltose; 2-25 mM methionine; and 0.01-0.2 mM EDTA.
N1835. 1-150 mg/mL EGFR/LGR5 BsAb; 3-25 mM citrate-phosphate buffer system providing a composition pH of 4-7.1; 10-180 mM sodium chloride; 150-350 mM maltose; 2-25 mM methionine; and 0.01-0.2 mM EDTA.
N1836. 10-60 mg/mL EGFR/LGR5 BsAb; 3-25 mM citrate-phosphate buffer system providing a composition pH of 4-7.1; 10-180 mM sodium chloride; 150-350 mM maltose; 2-25 mM methionine; and 0.01-0.2 mM EDTA.
N1837. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1838. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1839. 1-150 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; and 2-25 mM methionine.
N1840. 10-60 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; and 2-25 mM methionine.
N1841. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; and 2-25 mM methionine.
N1842. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; and 2-25 mM methionine.
N1843. 1-150 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; and 2-25 mM methionine.
N1844. 10-60 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; and 2-25 mM methionine.
N1845. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1846. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1847. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1848. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; and 2-25 mM methionine.
N1849. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1850. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1851. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1852. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1853. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1854. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1855. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1856. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1857. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1858. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1859. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1860. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; and 2-25 mM methionine.
N1861. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1862. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1863. 1-150 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1864. 10-60 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1865. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1866. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1867. 1-150 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1868. 10-60 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1869. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1870. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1871. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1872. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; and 0.01-0.2 mM EDTA.
N1873. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1874. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1875. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1876. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1877. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1878. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1879. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1880. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1881. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1882. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1883. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1884. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; and 0.01-0.2 mM EDTA.
N1885. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; 2-25 mM methionine; and 0.01-0.2 mM EDTA.
N1886. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; 2-25 mM methionine; and 0.01-0.2 mM EDTA.
N1887. 1-150 mg/mL EGFR/LGR5 BsAb; 3-25 mM acetate buffer system providing a composition pH of 4-5.6; 10-180 mM sodium chloride; 0.4-1.2 mg/mL surfactant; 2-25 mM methionine; and 0.01-0.2 mM EDTA.
N1888. 10-60 mg/mL EGFR/LGR5 BsAb; 3-25 mM acetate buffer system providing a composition pH of 4-5.6; 10-180 mM sodium chloride; 0.4-1.2 mg/mL surfactant; 2-25 mM methionine; and 0.01-0.2 mM EDTA.
N1889. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; 2-25 mM methionine; and 0.01-0.2 mM EDTA.
N1890. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; 2-25 mM methionine; and 0.01-0.2 mM EDTA.
N1891. 1-150 mg/mL EGFR/LGR5 BsAb; 3-25 mM succinate buffer system providing a composition pH of 4-6.6; 10-180 mM sodium chloride; 0.4-1.2 mg/mL surfactant; 2-25 mM methionine; and 0.01-0.2 mM EDTA.
N1892. 10-60 mg/mL EGFR/LGR5 BsAb; 3-25 mM succinate buffer system providing a composition pH of 4-6.6; 10-180 mM sodium chloride; 0.4-1.2 mg/mL surfactant; 2-25 mM methionine; and 0.01-0.2 mM EDTA.
N1893. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; 2-25 mM methionine; and 0.01-0.2 mM EDTA.
N1894. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; 2-25 mM methionine; and 0.01-0.2 mM EDTA.
N1895. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; 2-25 mM methionine; and 0.01-0.2 mM EDTA.
N1896. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; 2-25 mM methionine; and 0.01-0.2 mM EDTA.
N1897. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; 2-25 mM methionine; and 0.01-0.2 mM EDTA.
N1898. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; 2-25 mM methionine; and 0.01-0.2 mM EDTA.
N1899. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; 2-25 mM methionine; and 0.01-0.2 mM EDTA.
N1900. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; 2-25 mM methionine; and 0.01-0.2 mM EDTA.
N1901. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; 2-25 mM methionine; and 0.01-0.2 mM EDTA.
N1902. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; 2-25 mM methionine; and 0.01-0.2 mM EDTA.
N1903. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; 2-25 mM methionine; and 0.01-0.2 mM EDTA.
N1904. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; 2-25 mM methionine; and 0.01-0.2 mM EDTA.
N1905. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 2-25 mM methionine; and 0.01-0.2 mM EDTA.
N1906. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 2-25 mM methionine; and 0.01-0.2 mM EDTA.
N1907. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 2-25 mM methionine; and 0.01-0.2 mM EDTA.
N1908. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 2-25 mM methionine; and 0.01-0.2 mM EDTA.
N1909. 1-150 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; 2-25 mM methionine; and 0.01-0.2 mM EDTA.
N1910. 10-60 mg/mL EGFR/LGR5 BsAb; 3-25 mM histidine buffer system providing a composition pH of 5.4-7.1; 10-180 mM sodium chloride; 5-150 mM amino acid component selected from the group consisting of proline, glycine, lysine, and any combination thereof; 2-25 mM methionine; and 0.01-0.2 mM EDTA.
N1911. 1-150 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; 2-25 mM methionine; and 0.01-0.2 mM EDTA.
N1912. 10-60 mg/mL EGFR/LGR5 BsAb; 3-25 mM acetate buffer system providing a composition pH of 4-5.6; 10-180 mM sodium chloride; 5-150 mM amino acid component selected from the group consisting of proline, glycine, lysine, and any combination thereof; 2-25 mM methionine; and 0.01-0.2 mM EDTA.
N1913. 1-150 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; 2-25 mM methionine; and 0.01-0.2 mM EDTA.
N1914. 10-60 mg/mL EGFR/LGR5 BsAb; 3-25 mM citrate buffer system providing a composition pH of 4.5-7.5; 10-180 mM sodium chloride; 5-150 mM amino acid component selected from the group consisting of proline, glycine, lysine, and any combination thereof; 2-25 mM methionine; and 0.01-0.2 mM EDTA.
N1915. 1-150 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; 2-25 mM methionine; and 0.01-0.2 mM EDTA.
N1916. 10-60 mg/mL EGFR/LGR5 BsAb; 3-25 mM succinate buffer system providing a composition pH of 4-6.6; 10-180 mM sodium chloride; 5-150 mM amino acid component selected from the group consisting of proline, glycine, lysine, and any combination thereof; 2-25 mM methionine; and 0.01-0.2 mM EDTA.
N1917. 1-150 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; 2-25 mM methionine; and 0.01-0.2 mM EDTA.
N1918. 10-60 mg/mL EGFR/LGR5 BsAb; 3-25 mM phosphate buffer system providing a composition pH of 5.8-8.1; 10-180 mM sodium chloride; 5-150 mM amino acid component selected from the group consisting of proline, glycine, lysine, and any combination thereof; 2-25 mM methionine; and 0.01-0.2 mM EDTA.
N1919. 1-150 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; 2-25 mM methionine; and 0.01-0.2 mM EDTA.
N1920. 10-60 mg/mL EGFR/LGR5 BsAb; 3-25 mM citrate-phosphate buffer system providing a composition pH of 4-7.1; 10-180 mM sodium chloride; 5-150 mM amino acid component selected from the group consisting of proline, glycine, lysine, and any combination thereof; 2-25 mM methionine; and 0.01-0.2 mM EDTA.
N1921. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1922. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1923. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1924. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1925. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1926. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1927. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1928. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1929. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1930. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1931. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1932. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N1933. 1-150 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; and 2-25 mM methionine.
N1934. 10-60 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; and 2-25 mM methionine.
N1935. 1-150 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; and 2-25 mM methionine.
N1936. 10-60 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; and 2-25 mM methionine.
N1937. 1-150 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; and 2-25 mM methionine.
N1938. 10-60 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; and 2-25 mM methionine.
N1939. 1-150 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; and 2-25 mM methionine.
N1940. 10-60 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; and 2-25 mM methionine.
N1941. 1-150 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; and 2-25 mM methionine.
N1942. 10-60 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; and 2-25 mM methionine.
N1943. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; and 2-25 mM methionine.
N1944. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; and 2-25 mM methionine.
N1945. 1-150 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; and 2-25 mM methionine.
N1946. 10-60 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; and 2-25 mM methionine.
N1947. 1-150 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; and 2-25 mM methionine.
N1948. 10-60 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; and 2-25 mM methionine.
N1949. 1-150 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; and 2-25 mM methionine.
N1950. 10-60 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; and 2-25 mM methionine.
N1951. 1-150 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; and 2-25 mM methionine.
N1952. 10-60 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; and 2-25 mM methionine.
N1953. 1-150 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; and 2-25 mM methionine.
N1954. 10-60 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; and 2-25 mM methionine.
N1955. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; and 2-25 mM methionine.
N1956. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; and 2-25 mM methionine.
N1957. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1958. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1959. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1960. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1961. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1962. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1963. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1964. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1965. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1966. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1967. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1968. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N1969. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1970. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1971. 1-150 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1972. 10-60 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1973. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1974. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1975. 1-150 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1976. 10-60 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1977. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1978. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1979. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1980. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; and 2-25 mM methionine.
N1981. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1982. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1983. 1-150 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; and 2-25 mM methionine.
N1984. 10-60 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; and 2-25 mM methionine.
N1985. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; and 2-25 mM methionine.
N1986. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; and 2-25 mM methionine.
N1987. 1-150 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; and 2-25 mM methionine.
N1988. 10-60 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; and 2-25 mM methionine.
N1989. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1990. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1991. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1992. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; and 2-25 mM methionine.
N1993. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1994. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1995. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1996. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1997. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1998. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N1999. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2000. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2001. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2002. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2003. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2004. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2005. 1-150 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2006. 10-60 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2007. 1-150 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2008. 10-60 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2009. 1-150 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2010. 10-60 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2011. 1-150 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2012. 10-60 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2013. 1-150 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2014. 10-60 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2015. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2016. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2017. 1-150 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2018. 10-60 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2019. 1-150 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2020. 10-60 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2021. 1-150 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2022. 10-60 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2023. 1-150 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2024. 10-60 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2025. 1-150 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2026. 10-60 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2027. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2028. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2029. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2030. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2031. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2032. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2033. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2034. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2035. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2036. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2037. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2038. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2039. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2040. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2041. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2042. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2043. 1-150 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2044. 10-60 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2045. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2046. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2047. 1-150 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2048. 10-60 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2049. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2050. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2051. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2052. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2053. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2054. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2055. 1-150 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2056. 10-60 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2057. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2058. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2059. 1-150 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2060. 10-60 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2061. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2062. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2063. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2064. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2065. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2066. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2067. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2068. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2069. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2070. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2071. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2072. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2073. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2074. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2075. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2076. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2077. 1-150 mg/mL EGFR/LGR5 BsAb; 3-25 mM histidine buffer system providing a composition pH of 5.4-7.1; 150-350 mM sucrose; 0.4-1.2 mg/mL surfactant; 2-25 mM methionine; and 0.01-0.2 mM EDTA.
N2078. 10-60 mg/mL EGFR/LGR5 BsAb; 3-25 mM histidine buffer system providing a composition pH of 5.4-7.1; 150-350 mM sucrose; 0.4-1.2 mg/mL surfactant; 2-25 mM methionine; and 0.01-0.2 mM EDTA.
N2079. 1-150 mg/mL EGFR/LGR5 BsAb; 3-25 mM acetate buffer system providing a composition pH of 4-5.6; 150-350 mM sucrose; 0.4-1.2 mg/mL surfactant; 2-25 mM methionine; and 0.01-0.2 mM EDTA.
N2080. 10-60 mg/mL EGFR/LGR5 BsAb; 3-25 mM acetate buffer system providing a composition pH of 4-5.6; 150-350 mM sucrose; 0.4-1.2 mg/mL surfactant; 2-25 mM methionine; and 0.01-0.2 mM EDTA.
N2081. 1-150 mg/mL EGFR/LGR5 BsAb; 3-25 mM citrate buffer system providing a composition pH of 4.5-7.5; 150-350 mM sucrose; 0.4-1.2 mg/mL surfactant; 2-25 mM methionine; and 0.01-0.2 mM EDTA.
N2082. 10-60 mg/mL EGFR/LGR5 BsAb; 3-25 mM citrate buffer system providing a composition pH of 4.5-7.5; 150-350 mM sucrose; 0.4-1.2 mg/mL surfactant; 2-25 mM methionine; and 0.01-0.2 mM EDTA.
N2083. 1-150 mg/mL EGFR/LGR5 BsAb; 3-25 mM succinate buffer system providing a composition pH of 4-6.6; 150-350 mM sucrose; 0.4-1.2 mg/mL surfactant; 2-25 mM methionine; and 0.01-0.2 mM EDTA.
N2084. 10-60 mg/mL EGFR/LGR5 BsAb; 3-25 mM succinate buffer system providing a composition pH of 4-6.6; 150-350 mM sucrose; 0.4-1.2 mg/mL surfactant; 2-25 mM methionine; and 0.01-0.2 mM EDTA.
N2085. 1-150 mg/mL EGFR/LGR5 BsAb; 3-25 mM phosphate buffer system providing a composition pH of 5.8-8.1; 150-350 mM sucrose; 0.4-1.2 mg/mL surfactant; 2-25 mM methionine; and 0.01-0.2 mM EDTA.
N2086. 10-60 mg/mL EGFR/LGR5 BsAb; 3-25 mM phosphate buffer system providing a composition pH of 5.8-8.1; 150-350 mM sucrose; 0.4-1.2 mg/mL surfactant; 2-25 mM methionine; and 0.01-0.2 mM EDTA.
N2087. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; 2-25 mM methionine; and 0.01-0.2 mM EDTA.
N2088. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; 2-25 mM methionine; and 0.01-0.2 mM EDTA.
N2089. 1-150 mg/mL EGFR/LGR5 BsAb; 3-25 mM histidine buffer system providing a composition pH of 5.4-7.1; 150-350 mM maltose; 0.4-1.2 mg/mL surfactant; 2-25 mM methionine; and 0.01-0.2 mM EDTA.
N2090. 10-60 mg/mL EGFR/LGR5 BsAb; 3-25 mM histidine buffer system providing a composition pH of 5.4-7.1; 150-350 mM maltose; 0.4-1.2 mg/mL surfactant; 2-25 mM methionine; and 0.01-0.2 mM EDTA.
N2091. 1-150 mg/mL EGFR/LGR5 BsAb; 3-25 mM acetate buffer system providing a composition pH of 4-5.6; 150-350 mM maltose; 0.4-1.2 mg/mL surfactant; 2-25 mM methionine; and 0.01-0.2 mM EDTA.
N2092. 10-60 mg/mL EGFR/LGR5 BsAb; 3-25 mM acetate buffer system providing a composition pH of 4-5.6; 150-350 mM maltose; 0.4-1.2 mg/mL surfactant; 2-25 mM methionine; and 0.01-0.2 mM EDTA.
N2093. 1-150 mg/mL EGFR/LGR5 BsAb; 3-25 mM citrate buffer system providing a composition pH of 4.5-7.5; 150-350 mM maltose; 0.4-1.2 mg/mL surfactant; 2-25 mM methionine; and 0.01-0.2 mM EDTA.
N2094. 10-60 mg/mL EGFR/LGR5 BsAb; 3-25 mM citrate buffer system providing a composition pH of 4.5-7.5; 150-350 mM maltose; 0.4-1.2 mg/mL surfactant; 2-25 mM methionine; and 0.01-0.2 mM EDTA.
N2095. 1-150 mg/mL EGFR/LGR5 BsAb; 3-25 mM succinate buffer system providing a composition pH of 4-6.6; 150-350 mM maltose; 0.4-1.2 mg/mL surfactant; 2-25 mM methionine; and 0.01-0.2 mM EDTA.
N2096. 10-60 mg/mL EGFR/LGR5 BsAb; 3-25 mM succinate buffer system providing a composition pH of 4-6.6; 150-350 mM maltose; 0.4-1.2 mg/mL surfactant; 2-25 mM methionine; and 0.01-0.2 mM EDTA.
N2097. 1-150 mg/mL EGFR/LGR5 BsAb; 3-25 mM phosphate buffer system providing a composition pH of 5.8-8.1; 150-350 mM maltose; 0.4-1.2 mg/mL surfactant; 2-25 mM methionine; and 0.01-0.2 mM EDTA.
N2098. 10-60 mg/mL EGFR/LGR5 BsAb; 3-25 mM phosphate buffer system providing a composition pH of 5.8-8.1; 150-350 mM maltose; 0.4-1.2 mg/mL surfactant; 2-25 mM methionine; and 0.01-0.2 mM EDTA.
N2099. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; 2-25 mM methionine; and 0.01-0.2 mM EDTA.
N2100. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; 2-25 mM methionine; and 0.01-0.2 mM EDTA.
N2101. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2102. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2103. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2104. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2105. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2106. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2107. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2108. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2109. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2110. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2111. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2112. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2113. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; 2-25 mM methionine; and 0.01-0.2 mM EDTA.
N2114. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; 2-25 mM methionine; and 0.01-0.2 mM EDTA.
N2115. 1-150 mg/mL EGFR/LGR5 BsAb; 3-25 mM acetate buffer system providing a composition pH of 4-5.6; 150-350 mM sucrose; 0.1-1.1 mg/mL polysorbate 80; 2-25 mM methionine; and 0.01-0.2 mM EDTA.
N2116. 10-60 mg/mL EGFR/LGR5 BsAb; 3-25 mM acetate buffer system providing a composition pH of 4-5.6; 150-350 mM sucrose; 0.1-1.1 mg/mL polysorbate 80; 2-25 mM methionine; and 0.01-0.2 mM EDTA.
N2117. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; 2-25 mM methionine; and 0.01-0.2 mM EDTA.
N2118. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; 2-25 mM methionine; and 0.01-0.2 mM EDTA.
N2119. 1-150 mg/mL EGFR/LGR5 BsAb; 3-25 mM succinate buffer system providing a composition pH of 4-6.6; 150-350 mM sucrose; 0.1-1.1 mg/mL polysorbate 80; 2-25 mM methionine; and 0.01-0.2 mM EDTA.
N2120. 10-60 mg/mL EGFR/LGR5 BsAb; 3-25 mM succinate buffer system providing a composition pH of 4-6.6; 150-350 mM sucrose; 0.1-1.1 mg/mL polysorbate 80; 2-25 mM methionine; and 0.01-0.2 mM EDTA.
N2121. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; 2-25 mM methionine; and 0.01-0.2 mM EDTA.
N2122. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; 2-25 mM methionine; and 0.01-0.2 mM EDTA.
N2123. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 2-25 mM methionine; and 0.01-0.2 mM EDTA.
N2124. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 2-25 mM methionine; and 0.01-0.2 mM EDTA.
N2125. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; 2-25 mM methionine; and 0.01-0.2 mM EDTA.
N2126. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; 2-25 mM methionine; and 0.01-0.2 mM EDTA.
N2127. 1-150 mg/mL EGFR/LGR5 BsAb; 3-25 mM acetate buffer system providing a composition pH of 4-5.6; 150-350 mM maltose; 0.1-1.1 mg/mL polysorbate 80; 2-25 mM methionine; and 0.01-0.2 mM EDTA.
N2128. 10-60 mg/mL EGFR/LGR5 BsAb; 3-25 mM acetate buffer system providing a composition pH of 4-5.6; 150-350 mM maltose; 0.1-1.1 mg/mL polysorbate 80; 2-25 mM methionine; and 0.01-0.2 mM EDTA.
N2129. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; 2-25 mM methionine; and 0.01-0.2 mM EDTA.
N2130. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; 2-25 mM methionine; and 0.01-0.2 mM EDTA.
N2131. 1-150 mg/mL EGFR/LGR5 BsAb; 3-25 mM succinate buffer system providing a composition pH of 4-6.6; 150-350 mM maltose; 0.1-1.1 mg/mL polysorbate 80; 2-25 mM methionine; and 0.01-0.2 mM EDTA.
N2132. 10-60 mg/mL EGFR/LGR5 BsAb; 3-25 mM succinate buffer system providing a composition pH of 4-6.6; 150-350 mM maltose; 0.1-1.1 mg/mL polysorbate 80; 2-25 mM methionine; and 0.01-0.2 mM EDTA.
N2133. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; 2-25 mM methionine; and 0.01-0.2 mM EDTA.
N2134. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; 2-25 mM methionine; and 0.01-0.2 mM EDTA.
N2135. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; 2-25 mM methionine; and 0.01-0.2 mM EDTA.
N2136. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; 2-25 mM methionine; and 0.01-0.2 mM EDTA.
N2137. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2138. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2139. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2140. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2141. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2142. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2143. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2144. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2145. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2146. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2147. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2148. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2149. 1-150 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; 2-25 mM methionine; and 0.01-0.2 mM EDTA.
N2150. 10-60 mg/mL EGFR/LGR5 BsAb; 3-25 mM histidine buffer system providing a composition pH of 5.4-7.1; 150-350 mM sucrose; 5-150 mM amino acid component selected from the group consisting of proline, glycine, lysine, and any combination thereof; 2-25 mM methionine; and 0.01-0.2 mM EDTA.
N2151. 1-150 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; 2-25 mM methionine; and 0.01-0.2 mM EDTA.
N2152. 10-60 mg/mL EGFR/LGR5 BsAb; 3-25 mM acetate buffer system providing a composition pH of 4-5.6; 150-350 mM sucrose; 5-150 mM amino acid component selected from the group consisting of proline, glycine, lysine, and any combination thereof; 2-25 mM methionine; and 0.01-0.2 mM EDTA.
N2153. 1-150 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; 2-25 mM methionine; and 0.01-0.2 mM EDTA.
N2154. 10-60 mg/mL EGFR/LGR5 BsAb; 3-25 mM citrate buffer system providing a composition pH of 4.5-7.5; 150-350 mM sucrose; 5-150 mM amino acid component selected from the group consisting of proline, glycine, lysine, and any combination thereof; 2-25 mM methionine; and 0.01-0.2 mM EDTA.
N2155. 1-150 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; 2-25 mM methionine; and 0.01-0.2 mM EDTA.
N2156. 10-60 mg/mL EGFR/LGR5 BsAb; 3-25 mM succinate buffer system providing a composition pH of 4-6.6; 150-350 mM sucrose; 5-150 mM amino acid component selected from the group consisting of proline, glycine, lysine, and any combination thereof; 2-25 mM methionine; and 0.01-0.2 mM EDTA.
N2157. 1-150 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; 2-25 mM methionine; and 0.01-0.2 mM EDTA.
N2158. 10-60 mg/mL EGFR/LGR5 BsAb; 3-25 mM phosphate buffer system providing a composition pH of 5.8-8.1; 150-350 mM sucrose; 5-150 mM amino acid component selected from the group consisting of proline, glycine, lysine, and any combination thereof; 2-25 mM methionine; and 0.01-0.2 mM EDTA.
N2159. 1-150 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; 2-25 mM methionine; and 0.01-0.2 mM EDTA.
N2160. 10-60 mg/mL EGFR/LGR5 BsAb; 3-25 mM citrate-phosphate buffer system providing a composition pH of 4-7.1; 150-350 mM sucrose; 5-150 mM amino acid component selected from the group consisting of proline, glycine, lysine, and any combination thereof; 2-25 mM methionine; and 0.01-0.2 mM EDTA.
N2161. 1-150 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; 2-25 mM methionine; and 0.01-0.2 mM EDTA.
N2162. 10-60 mg/mL EGFR/LGR5 BsAb; 3-25 mM histidine buffer system providing a composition pH of 5.4-7.1; 150-350 mM maltose; 5-150 mM amino acid component selected from the group consisting of proline, glycine, lysine, and any combination thereof; 2-25 mM methionine; and 0.01-0.2 mM EDTA.
N2163. 1-150 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; 2-25 mM methionine; and 0.01-0.2 mM EDTA.
N2164. 10-60 mg/mL EGFR/LGR5 BsAb; 3-25 mM acetate buffer system providing a composition pH of 4-5.6; 150-350 mM maltose; 5-150 mM amino acid component selected from the group consisting of proline, glycine, lysine, and any combination thereof; 2-25 mM methionine; and 0.01-0.2 mM EDTA.
N2165. 1-150 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; 2-25 mM methionine; and 0.01-0.2 mM EDTA.
N2166. 10-60 mg/mL EGFR/LGR5 BsAb; 3-25 mM citrate buffer system providing a composition pH of 4.5-7.5; 150-350 mM maltose; 5-150 mM amino acid component selected from the group consisting of proline, glycine, lysine, and any combination thereof; 2-25 mM methionine; and 0.01-0.2 mM EDTA.
N2167. 1-150 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; 2-25 mM methionine; and 0.01-0.2 mM EDTA.
N2168. 10-60 mg/mL EGFR/LGR5 BsAb; 3-25 mM succinate buffer system providing a composition pH of 4-6.6; 150-350 mM maltose; 5-150 mM amino acid component selected from the group consisting of proline, glycine, lysine, and any combination thereof; 2-25 mM methionine; and 0.01-0.2 mM EDTA.
N2169. 1-150 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; 2-25 mM methionine; and 0.01-0.2 mM EDTA.
N2170. 10-60 mg/mL EGFR/LGR5 BsAb; 3-25 mM phosphate buffer system providing a composition pH of 5.8-8.1; 150-350 mM maltose; 5-150 mM amino acid component selected from the group consisting of proline, glycine, lysine, and any combination thereof; 2-25 mM methionine; and 0.01-0.2 mM EDTA.
N2171. 1-150 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; 2-25 mM methionine; and 0.01-0.2 mM EDTA.
N2172. 10-60 mg/mL EGFR/LGR5 BsAb; 3-25 mM citrate-phosphate buffer system providing a composition pH of 4-7.1; 150-350 mM maltose; 5-150 mM amino acid component selected from the group consisting of proline, glycine, lysine, and any combination thereof; 2-25 mM methionine; and 0.01-0.2 mM EDTA.
N2173. 1-150 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2174. 10-60 mg/mL EGFR/LGR5 BsAb; 3-25 mM histidine buffer system providing a composition pH of 5.4-7.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2175. 1-150 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2176. 10-60 mg/mL EGFR/LGR5 BsAb; 3-25 mM acetate buffer system providing a composition pH of 4-5.6; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2177. 1-150 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2178. 10-60 mg/mL EGFR/LGR5 BsAb; 3-25 mM citrate buffer system providing a composition pH of 4.5-7.5; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2179. 1-150 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2180. 10-60 mg/mL EGFR/LGR5 BsAb; 3-25 mM succinate buffer system providing a composition pH of 4-6.6; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2181. 1-150 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2182. 10-60 mg/mL EGFR/LGR5 BsAb; 3-25 mM phosphate buffer system providing a composition pH of 5.8-8.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2183. 1-150 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2184. 10-60 mg/mL EGFR/LGR5 BsAb; 3-25 mM citrate-phosphate buffer system providing a composition pH of 4-7.1; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2185. 1-150 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2186. 10-60 mg/mL EGFR/LGR5 BsAb; 3-25 mM histidine buffer system providing a composition pH of 5.4-7.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2187. 1-150 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2188. 10-60 mg/mL EGFR/LGR5 BsAb; 3-25 mM acetate buffer system providing a composition pH of 4-5.6; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2189. 1-150 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2190. 10-60 mg/mL EGFR/LGR5 BsAb; 3-25 mM citrate buffer system providing a composition pH of 4.5-7.5; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2191. 1-150 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2192. 10-60 mg/mL EGFR/LGR5 BsAb; 3-25 mM succinate buffer system providing a composition pH of 4-6.6; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2193. 1-150 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2194. 10-60 mg/mL EGFR/LGR5 BsAb; 3-25 mM phosphate buffer system providing a composition pH of 5.8-8.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2195. 1-150 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2196. 10-60 mg/mL EGFR/LGR5 BsAb; 3-25 mM citrate-phosphate buffer system providing a composition pH of 4-7.1; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2197. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2198. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2199. 1-150 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2200. 10-60 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2201. 1-150 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2202. 10-60 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2203. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2204. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2205. 1-150 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2206. 10-60 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2207. 1-150 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2208. 10-60 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2209. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2210. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2211. 1-150 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2212. 10-60 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2213. 1-150 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2214. 10-60 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2215. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2216. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2217. 1-150 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2218. 10-60 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2219. 1-150 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2220. 10-60 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2221. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2222. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2223. 1-150 mg/mL EGFR/LGR5 BsAb; 10-180 mM sodium chloride; 150-350 mM sucrose; 0.4-1.2 mg/mL surfactant; 2-25 mM methionine; and 0.01-0.2 mM EDTA.
N2224. 10-60 mg/mL EGFR/LGR5 BsAb; 10-180 mM sodium chloride; 150-350 mM sucrose; 0.4-1.2 mg/mL surfactant; 2-25 mM methionine; and 0.01-0.2 mM EDTA.
N2225. 1-150 mg/mL EGFR/LGR5 BsAb; 10-180 mM sodium chloride; 150-350 mM maltose; 0.4-1.2 mg/mL surfactant; 2-25 mM methionine; and 0.01-0.2 mM EDTA.
N2226. 10-60 mg/mL EGFR/LGR5 BsAb; 10-180 mM sodium chloride; 150-350 mM maltose; 0.4-1.2 mg/mL surfactant; 2-25 mM methionine; and 0.01-0.2 mM EDTA.
N2227. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2228. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2229. 1-150 mg/mL EGFR/LGR5 BsAb; 10-180 mM sodium chloride; 150-350 mM sucrose; 0.1-1.1 mg/mL polysorbate 80; 2-25 mM methionine; and 0.01-0.2 mM EDTA.
N2230. 10-60 mg/mL EGFR/LGR5 BsAb; 10-180 mM sodium chloride; 150-350 mM sucrose; 0.1-1.1 mg/mL polysorbate 80; 2-25 mM methionine; and 0.01-0.2 mM EDTA.
N2231. 1-150 mg/mL EGFR/LGR5 BsAb; 10-180 mM sodium chloride; 150-350 mM maltose; 0.1-1.1 mg/mL polysorbate 80; 2-25 mM methionine; and 0.01-0.2 mM EDTA.
N2232. 10-60 mg/mL EGFR/LGR5 BsAb; 10-180 mM sodium chloride; 150-350 mM maltose; 0.1-1.1 mg/mL polysorbate 80; 2-25 mM methionine; and 0.01-0.2 mM EDTA.
N2233. 1-150 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2234. 10-60 mg/mL EGFR/LGR5 BsAb; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2235. 1-150 mg/mL EGFR/LGR5 BsAb; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2236. 10-60 mg/mL EGFR/LGR5 BsAb; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2237. 1-150 mg/mL EGFR/LGR5 BsAb; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2238. 10-60 mg/mL EGFR/LGR5 BsAb; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2239. 1-150 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2240. 10-60 mg/mL EGFR/LGR5 BsAb; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2241. 1-150 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2242. 10-60 mg/mL EGFR/LGR5 BsAb; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2243. 1-150 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2244. 10-60 mg/mL EGFR/LGR5 BsAb; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2245. 1-150 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2246. 10-60 mg/mL EGFR/LGR5 BsAb; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2247. 1-150 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2248. 10-60 mg/mL EGFR/LGR5 BsAb; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2249. 1-150 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2250. 10-60 mg/mL EGFR/LGR5 BsAb; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2251. 1-150 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2252. 10-60 mg/mL EGFR/LGR5 BsAb; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2253. 1-150 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2254. 10-60 mg/mL EGFR/LGR5 BsAb; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2255. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2256. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2257. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2258. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2259. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2260. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2261. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2262. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2263. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2264. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2265. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2266. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 2-25 mM methionine.
N2267. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2268. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2269. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2270. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2271. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2272. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2273. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2274. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2275. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2276. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2277. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2278. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 2-25 mM methionine.
N2279. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2280. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2281. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2282. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2283. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2284. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2285. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2286. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2287. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2288. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2289. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2290. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 2-25 mM methionine.
N2291. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2292. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2293. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2294. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2295. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2296. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2297. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2298. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2299. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2300. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2301. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2302. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 2-25 mM methionine.
N2303. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2304. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2305. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2306. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2307. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2308. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2309. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2310. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2311. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2312. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2313. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2314. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 2-25 mM methionine.
N2315. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2316. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2317. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2318. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2319. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2320. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2321. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2322. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2323. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2324. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2325. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2326. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 2-25 mM methionine.
N2327. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2328. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2329. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2330. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2331. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2332. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2333. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2334. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2335. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2336. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2337. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2338. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; and 0.01-0.2 mM EDTA.
N2339. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2340. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2341. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2342. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2343. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2344. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2345. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2346. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2347. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2348. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2349. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2350. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; and 0.01-0.2 mM EDTA.
N2351. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2352. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2353. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2354. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2355. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2356. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2357. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2358. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2359. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2360. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2361. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2362. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; and 0.01-0.2 mM EDTA.
N2363. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2364. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2365. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2366. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2367. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2368. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2369. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2370. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2371. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2372. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2373. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2374. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; and 0.01-0.2 mM EDTA.
N2375. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2376. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2377. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2378. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2379. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2380. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2381. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2382. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2383. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2384. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2385. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2386. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; and 0.01-0.2 mM EDTA.
N2387. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2388. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2389. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2390. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2391. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2392. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2393. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2394. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2395. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2396. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2397. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2398. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; and 0.01-0.2 mM EDTA.
N2399. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2400. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2401. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2402. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2403. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2404. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2405. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2406. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2407. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2408. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2409. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2410. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 2-25 mM methionine; and 0.01-0.2 mM EDTA.
N2411. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 2-25 mM methionine; and 0.01-0.2 mM EDTA.
N2412. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 2-25 mM methionine; and 0.01-0.2 mM EDTA.
N2413. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 2-25 mM methionine; and 0.01-0.2 mM EDTA.
N2414. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 2-25 mM methionine; and 0.01-0.2 mM EDTA.
N2415. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 2-25 mM methionine; and 0.01-0.2 mM EDTA.
N2416. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 2-25 mM methionine; and 0.01-0.2 mM EDTA.
N2417. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 2-25 mM methionine; and 0.01-0.2 mM EDTA.
N2418. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 2-25 mM methionine; and 0.01-0.2 mM EDTA.
N2419. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 2-25 mM methionine; and 0.01-0.2 mM EDTA.
N2420. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 2-25 mM methionine; and 0.01-0.2 mM EDTA.
N2421. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 2-25 mM methionine; and 0.01-0.2 mM EDTA.
N2422. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 2-25 mM methionine; and 0.01-0.2 mM EDTA.
N2423. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 2-25 mM methionine; and 0.01-0.2 mM EDTA.
N2424. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 2-25 mM methionine; and 0.01-0.2 mM EDTA.
N2425. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 2-25 mM methionine; and 0.01-0.2 mM EDTA.
N2426. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 2-25 mM methionine; and 0.01-0.2 mM EDTA.
N2427. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 2-25 mM methionine; and 0.01-0.2 mM EDTA.
N2428. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 2-25 mM methionine; and 0.01-0.2 mM EDTA.
N2429. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 2-25 mM methionine; and 0.01-0.2 mM EDTA.
N2430. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 2-25 mM methionine; and 0.01-0.2 mM EDTA.
N2431. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 2-25 mM methionine; and 0.01-0.2 mM EDTA.
N2432. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 2-25 mM methionine; and 0.01-0.2 mM EDTA.
N2433. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 2-25 mM methionine; and 0.01-0.2 mM EDTA.
N2434. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 2-25 mM methionine; and 0.01-0.2 mM EDTA.
N2435. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2436. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2437. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2438. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2439. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2440. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2441. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2442. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2443. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2444. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2445. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2446. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 2-25 mM methionine; and 0.01-0.2 mM EDTA.
N2447. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 2-25 mM methionine; and 0.01-0.2 mM EDTA.
N2448. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 2-25 mM methionine; and 0.01-0.2 mM EDTA.
N2449. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 2-25 mM methionine; and 0.01-0.2 mM EDTA.
N2450. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 2-25 mM methionine; and 0.01-0.2 mM EDTA.
N2451. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 2-25 mM methionine; and 0.01-0.2 mM EDTA.
N2452. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 2-25 mM methionine; and 0.01-0.2 mM EDTA.
N2453. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 2-25 mM methionine; and 0.01-0.2 mM EDTA.
N2454. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 2-25 mM methionine; and 0.01-0.2 mM EDTA.
N2455. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 2-25 mM methionine; and 0.01-0.2 mM EDTA.
N2456. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 2-25 mM methionine; and 0.01-0.2 mM EDTA.
N2457. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 2-25 mM methionine; and 0.01-0.2 mM EDTA.
N2458. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 2-25 mM methionine; and 0.01-0.2 mM EDTA.
N2459. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 2-25 mM methionine; and 0.01-0.2 mM EDTA.
N2460. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 2-25 mM methionine; and 0.01-0.2 mM EDTA.
N2461. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 2-25 mM methionine; and 0.01-0.2 mM EDTA.
N2462. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 2-25 mM methionine; and 0.01-0.2 mM EDTA.
N2463. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 2-25 mM methionine; and 0.01-0.2 mM EDTA.
N2464. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 2-25 mM methionine; and 0.01-0.2 mM EDTA.
N2465. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 2-25 mM methionine; and 0.01-0.2 mM EDTA.
N2466. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 2-25 mM methionine; and 0.01-0.2 mM EDTA.
N2467. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 2-25 mM methionine; and 0.01-0.2 mM EDTA.
N2468. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 2-25 mM methionine; and 0.01-0.2 mM EDTA.
N2469. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 2-25 mM methionine; and 0.01-0.2 mM EDTA.
N2470. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 2-25 mM methionine; and 0.01-0.2 mM EDTA.
N2471. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2472. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2473. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2474. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2475. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2476. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2477. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2478. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2479. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2480. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2481. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2482. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 5-150 mM amino acid component selected from the group consisting of proline, glycine, lysine, and any combination thereof; 2-25 mM methionine; and 0.01-0.2 mM EDTA.
N2483. 1-150 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2484. 10-60 mg/mL EGFR/LGR5 BsAb; 3-25 mM histidine buffer system providing a composition pH of 5.4-7.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2485. 1-150 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2486. 10-60 mg/mL EGFR/LGR5 BsAb; 3-25 mM acetate buffer system providing a composition pH of 4-5.6; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2487. 1-150 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2488. 10-60 mg/mL EGFR/LGR5 BsAb; 3-25 mM citrate buffer system providing a composition pH of 4.5-7.5; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2489. 1-150 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2490. 10-60 mg/mL EGFR/LGR5 BsAb; 3-25 mM succinate buffer system providing a composition pH of 4-6.6; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2491. 1-150 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2492. 10-60 mg/mL EGFR/LGR5 BsAb; 3-25 mM phosphate buffer system providing a composition pH of 5.8-8.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2493. 1-150 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2494. 10-60 mg/mL EGFR/LGR5 BsAb; 3-25 mM citrate-phosphate buffer system providing a composition pH of 4-7.1; 10-180 mM sodium chloride; 150-350 mM sucrose; 5-150 mM amino acid component selected from the group consisting of proline, glycine, lysine, and any combination thereof; 2-25 mM methionine; and 0.01-0.2 mM EDTA.
N2495. 1-150 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2496. 10-60 mg/mL EGFR/LGR5 BsAb; 3-25 mM histidine buffer system providing a composition pH of 5.4-7.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2497. 1-150 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2498. 10-60 mg/mL EGFR/LGR5 BsAb; 3-25 mM acetate buffer system providing a composition pH of 4-5.6; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2499. 1-150 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2500. 10-60 mg/mL EGFR/LGR5 BsAb; 3-25 mM citrate buffer system providing a composition pH of 4.5-7.5; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2501. 1-150 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2502. 10-60 mg/mL EGFR/LGR5 BsAb; 3-25 mM succinate buffer system providing a composition pH of 4-6.6; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2503. 1-150 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2504. 10-60 mg/mL EGFR/LGR5 BsAb; 3-25 mM phosphate buffer system providing a composition pH of 5.8-8.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2505. 1-150 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2506. 10-60 mg/mL EGFR/LGR5 BsAb; 3-25 mM citrate-phosphate buffer system providing a composition pH of 4-7.1; 10-180 mM sodium chloride; 150-350 mM maltose; 5-150 mM amino acid component selected from the group consisting of proline, glycine, lysine, and any combination thereof; 2-25 mM methionine; and 0.01-0.2 mM EDTA.
N2507. 1-150 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2508. 10-60 mg/mL EGFR/LGR5 BsAb; 3-25 mM histidine buffer system providing a composition pH of 5.4-7.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2509. 1-150 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2510. 10-60 mg/mL EGFR/LGR5 BsAb; 3-25 mM acetate buffer system providing a composition pH of 4-5.6; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2511. 1-150 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2512. 10-60 mg/mL EGFR/LGR5 BsAb; 3-25 mM citrate buffer system providing a composition pH of 4.5-7.5; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2513. 1-150 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2514. 10-60 mg/mL EGFR/LGR5 BsAb; 3-25 mM succinate buffer system providing a composition pH of 4-6.6; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2515. 1-150 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2516. 10-60 mg/mL EGFR/LGR5 BsAb; 3-25 mM phosphate buffer system providing a composition pH of 5.8-8.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2517. 1-150 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2518. 10-60 mg/mL EGFR/LGR5 BsAb; 3-25 mM citrate-phosphate buffer system providing a composition pH of 4-7.1; 10-180 mM sodium chloride; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2519. 1-150 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2520. 10-60 mg/mL EGFR/LGR5 BsAb; 3-25 mM histidine buffer system providing a composition pH of 5.4-7.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2521. 1-150 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2522. 10-60 mg/mL EGFR/LGR5 BsAb; 3-25 mM acetate buffer system providing a composition pH of 4-5.6; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2523. 1-150 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2524. 10-60 mg/mL EGFR/LGR5 BsAb; 3-25 mM citrate buffer system providing a composition pH of 4.5-7.5; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2525. 1-150 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2526. 10-60 mg/mL EGFR/LGR5 BsAb; 3-25 mM succinate buffer system providing a composition pH of 4-6.6; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2527. 1-150 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2528. 10-60 mg/mL EGFR/LGR5 BsAb; 3-25 mM phosphate buffer system providing a composition pH of 5.8-8.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2529. 1-150 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2530. 10-60 mg/mL EGFR/LGR5 BsAb; 3-25 mM citrate-phosphate buffer system providing a composition pH of 4-7.1; 10-180 mM sodium chloride; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2531. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2532. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2533. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2534. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2535. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2536. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2537. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2538. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2539. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2540. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2541. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2542. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2543. 1-150 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2544. 10-60 mg/mL EGFR/LGR5 BsAb; 3-25 mM histidine buffer system providing a composition pH of 5.4-7.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2545. 1-150 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2546. 10-60 mg/mL EGFR/LGR5 BsAb; 3-25 mM acetate buffer system providing a composition pH of 4-5.6; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2547. 1-150 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2548. 10-60 mg/mL EGFR/LGR5 BsAb; 3-25 mM citrate buffer system providing a composition pH of 4.5-7.5; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2549. 1-150 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2550. 10-60 mg/mL EGFR/LGR5 BsAb; 3-25 mM succinate buffer system providing a composition pH of 4-6.6; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2551. 1-150 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2552. 10-60 mg/mL EGFR/LGR5 BsAb; 3-25 mM phosphate buffer system providing a composition pH of 5.8-8.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2553. 1-150 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2554. 10-60 mg/mL EGFR/LGR5 BsAb; 3-25 mM citrate-phosphate buffer system providing a composition pH of 4-7.1; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2555. 1-150 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2556. 10-60 mg/mL EGFR/LGR5 BsAb; 3-25 mM histidine buffer system providing a composition pH of 5.4-7.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2557. 1-150 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2558. 10-60 mg/mL EGFR/LGR5 BsAb; 3-25 mM acetate buffer system providing a composition pH of 4-5.6; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2559. 1-150 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2560. 10-60 mg/mL EGFR/LGR5 BsAb; 3-25 mM citrate buffer system providing a composition pH of 4.5-7.5; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2561. 1-150 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2562. 10-60 mg/mL EGFR/LGR5 BsAb; 3-25 mM succinate buffer system providing a composition pH of 4-6.6; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2563. 1-150 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2564. 10-60 mg/mL EGFR/LGR5 BsAb; 3-25 mM phosphate buffer system providing a composition pH of 5.8-8.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2565. 1-150 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2566. 10-60 mg/mL EGFR/LGR5 BsAb; 3-25 mM citrate-phosphate buffer system providing a composition pH of 4-7.1; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2567. 1-150 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2568. 10-60 mg/mL EGFR/LGR5 BsAb; 3-25 mM histidine buffer system providing a composition pH of 5.4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2569. 1-150 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2570. 10-60 mg/mL EGFR/LGR5 BsAb; 3-25 mM acetate buffer system providing a composition pH of 4-5.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2571. 1-150 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2572. 10-60 mg/mL EGFR/LGR5 BsAb; 3-25 mM citrate buffer system providing a composition pH of 4.5-7.5; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2573. 1-150 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2574. 10-60 mg/mL EGFR/LGR5 BsAb; 3-25 mM succinate buffer system providing a composition pH of 4-6.6; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2575. 1-150 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2576. 10-60 mg/mL EGFR/LGR5 BsAb; 3-25 mM phosphate buffer system providing a composition pH of 5.8-8.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2577. 1-150 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2578. 10-60 mg/mL EGFR/LGR5 BsAb; 3-25 mM citrate-phosphate buffer system providing a composition pH of 4-7.1; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2579. 1-150 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2580. 10-60 mg/mL EGFR/LGR5 BsAb; 3-25 mM histidine buffer system providing a composition pH of 5.4-7.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2581. 1-150 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2582. 10-60 mg/mL EGFR/LGR5 BsAb; 3-25 mM acetate buffer system providing a composition pH of 4-5.6; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2583. 1-150 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2584. 10-60 mg/mL EGFR/LGR5 BsAb; 3-25 mM citrate buffer system providing a composition pH of 4.5-7.5; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2585. 1-150 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2586. 10-60 mg/mL EGFR/LGR5 BsAb; 3-25 mM succinate buffer system providing a composition pH of 4-6.6; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2587. 1-150 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2588. 10-60 mg/mL EGFR/LGR5 BsAb; 3-25 mM phosphate buffer system providing a composition pH of 5.8-8.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2589. 1-150 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2590. 10-60 mg/mL EGFR/LGR5 BsAb; 3-25 mM citrate-phosphate buffer system providing a composition pH of 4-7.1; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2591. 1-150 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2592. 10-60 mg/mL EGFR/LGR5 BsAb; 3-25 mM histidine buffer system providing a composition pH of 5.4-7.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2593. 1-150 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2594. 10-60 mg/mL EGFR/LGR5 BsAb; 3-25 mM acetate buffer system providing a composition pH of 4-5.6; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2595. 1-150 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2596. 10-60 mg/mL EGFR/LGR5 BsAb; 3-25 mM citrate buffer system providing a composition pH of 4.5-7.5; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2597. 1-150 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2598. 10-60 mg/mL EGFR/LGR5 BsAb; 3-25 mM succinate buffer system providing a composition pH of 4-6.6; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2599. 1-150 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2600. 10-60 mg/mL EGFR/LGR5 BsAb; 3-25 mM phosphate buffer system providing a composition pH of 5.8-8.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2601. 1-150 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2602. 10-60 mg/mL EGFR/LGR5 BsAb; 3-25 mM citrate-phosphate buffer system providing a composition pH of 4-7.1; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2603. 1-150 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2604. 10-60 mg/mL EGFR/LGR5 BsAb; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2605. 1-150 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2606. 10-60 mg/mL EGFR/LGR5 BsAb; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2607. 1-150 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2608. 10-60 mg/mL EGFR/LGR5 BsAb; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2609. 1-150 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2610. 10-60 mg/mL EGFR/LGR5 BsAb; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2611. 1-150 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2612. 10-60 mg/mL EGFR/LGR5 BsAb; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2613. 1-150 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2614. 10-60 mg/mL EGFR/LGR5 BsAb; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2615. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2616. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2617. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2618. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2619. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2620. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2621. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2622. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2623. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2624. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2625. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2626. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2627. 1-150 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2628. 10-60 mg/mL EGFR/LGR5 BsAb; 3-25 mM histidine buffer system providing a composition pH of 5.4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2629. 1-150 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2630. 10-60 mg/mL EGFR/LGR5 BsAb; 3-25 mM acetate buffer system providing a composition pH of 4-5.6; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2631. 1-150 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2632. 10-60 mg/mL EGFR/LGR5 BsAb; 3-25 mM citrate buffer system providing a composition pH of 4.5-7.5; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2633. 1-150 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2634. 10-60 mg/mL EGFR/LGR5 BsAb; 3-25 mM succinate buffer system providing a composition pH of 4-6.6; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2635. 1-150 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2636. 10-60 mg/mL EGFR/LGR5 BsAb; 3-25 mM phosphate buffer system providing a composition pH of 5.8-8.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2637. 1-150 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2638. 10-60 mg/mL EGFR/LGR5 BsAb; 3-25 mM citrate-phosphate buffer system providing a composition pH of 4-7.1; 10-180 mM sodium chloride; 150-350 mM sucrose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2639. 1-150 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2640. 10-60 mg/mL EGFR/LGR5 BsAb; 3-25 mM histidine buffer system providing a composition pH of 5.4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2641. 1-150 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2642. 10-60 mg/mL EGFR/LGR5 BsAb; 3-25 mM acetate buffer system providing a composition pH of 4-5.6; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2643. 1-150 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2644. 10-60 mg/mL EGFR/LGR5 BsAb; 3-25 mM citrate buffer system providing a composition pH of 4.5-7.5; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2645. 1-150 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2646. 10-60 mg/mL EGFR/LGR5 BsAb; 3-25 mM succinate buffer system providing a composition pH of 4-6.6; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2647. 1-150 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2648. 10-60 mg/mL EGFR/LGR5 BsAb; 3-25 mM phosphate buffer system providing a composition pH of 5.8-8.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2649. 1-150 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2650. 10-60 mg/mL EGFR/LGR5 BsAb; 3-25 mM citrate-phosphate buffer system providing a composition pH of 4-7.1; 10-180 mM sodium chloride; 150-350 mM maltose; 0.4-1.2 mg/mL surfactant; 5-150 mM amino acid component selected from the group consisting of proline, glycine, lysine, and any combination thereof; 2-25 mM methionine; and 0.01-0.2 mM EDTA.
N2651. 1-150 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2652. 10-60 mg/mL EGFR/LGR5 BsAb; 3-25 mM histidine buffer system providing a composition pH of 5.4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2653. 1-150 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2654. 10-60 mg/mL EGFR/LGR5 BsAb; 3-25 mM acetate buffer system providing a composition pH of 4-5.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2655. 1-150 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2656. 10-60 mg/mL EGFR/LGR5 BsAb; 3-25 mM citrate buffer system providing a composition pH of 4.5-7.5; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2657. 1-150 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2658. 10-60 mg/mL EGFR/LGR5 BsAb; 3-25 mM succinate buffer system providing a composition pH of 4-6.6; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2659. 1-150 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2660. 10-60 mg/mL EGFR/LGR5 BsAb; 3-25 mM phosphate buffer system providing a composition pH of 5.8-8.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2661. 1-150 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2662. 10-60 mg/mL EGFR/LGR5 BsAb; 3-25 mM citrate-phosphate buffer system providing a composition pH of 4-7.1; 10-180 mM sodium chloride; 100-400 mM sugar component selected from the group consisting of sucrose, mannitol, sorbitol, and maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2663. 1-150 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2664. 10-60 mg/mL EGFR/LGR5 BsAb; 3-25 mM histidine buffer system providing a composition pH of 5.4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2665. 1-150 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2666. 10-60 mg/mL EGFR/LGR5 BsAb; 3-25 mM acetate buffer system providing a composition pH of 4-5.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2667. 1-150 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2668. 10-60 mg/mL EGFR/LGR5 BsAb; 3-25 mM citrate buffer system providing a composition pH of 4.5-7.5; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2669. 1-150 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2670. 10-60 mg/mL EGFR/LGR5 BsAb; 3-25 mM succinate buffer system providing a composition pH of 4-6.6; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2671. 1-150 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2672. 10-60 mg/mL EGFR/LGR5 BsAb; 3-25 mM phosphate buffer system providing a composition pH of 5.8-8.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2673. 1-150 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2674. 10-60 mg/mL EGFR/LGR5 BsAb; 3-25 mM citrate-phosphate buffer system providing a composition pH of 4-7.1; 10-180 mM sodium chloride; 150-350 mM sucrose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2675. 1-150 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2676. 10-60 mg/mL EGFR/LGR5 BsAb; 3-25 mM histidine buffer system providing a composition pH of 5.4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2677. 1-150 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2678. 10-60 mg/mL EGFR/LGR5 BsAb; 3-25 mM acetate buffer system providing a composition pH of 4-5.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2679. 1-150 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2680. 10-60 mg/mL EGFR/LGR5 BsAb; 3-25 mM citrate buffer system providing a composition pH of 4.5-7.5; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2681. 1-150 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2682. 10-60 mg/mL EGFR/LGR5 BsAb; 3-25 mM succinate buffer system providing a composition pH of 4-6.6; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2683. 1-150 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2684. 10-60 mg/mL EGFR/LGR5 BsAb; 3-25 mM phosphate buffer system providing a composition pH of 5.8-8.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2685. 1-150 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.
N2686. 10-60 mg/mL EGFR/LGR5 BsAb; 3-25 mM citrate-phosphate buffer system providing a composition pH of 4-7.1; 10-180 mM sodium chloride; 150-350 mM maltose; 0.1-1.1 mg/mL polysorbate 80; 5-150 mM amino acid component selected from the group consisting of proline, glycine, lysine, and any combination thereof; 2-25 mM methionine; and 0.01-0.2 mM EDTA.

### Package

The present invention provides a package comprising a pharmaceutical composition as defined herein. Preferably the package comprises a drug delivery device as defined herein, preferably a plurality of drug delivery devices. The package may comprise any suitable container for containing one or more drug delivery devices.

The present invention provides a method of manufacturing a package, the method comprising incorporating a pharmaceutical composition as defined herein within a package. Suitably this is achieved by incorporating said pharmaceutical composition within one or more drug delivery devices, and thereafter incorporating the one or more pre-filled drug delivery devices into a container present within the package.

The present invention provides a package obtainable by, obtained by, or directly obtained by a method of manufacture defined herein.

### Drug-Delivery Device

The present invention provides a drug delivery device comprising a pharmaceutical composition as defined herein. Preferably the drug delivery device comprises a chamber, preferably made of glass, within which the, preferably liquid (most preferably aqueous), pharmaceutical composition resides. Preferably the drug delivery device is sterile.

The drug delivery device may be a vial, ampoule, syringe, injection pen (e.g. essentially incorporating a syringe), autoinjector, or intravenous bag. Most preferably the drug delivery device is a syringe. Preferably the syringe comprises a needle.

The present invention provides a method of manufacturing a drug delivery device, preferably as defined herein, the method comprising incorporating a pharmaceutical composition as defined herein within a drug delivery device. Such manufacture typically involves charging the pharmaceutical composition as defined herein to a syringe, preferably via a needle affixed thereto. The needle may thereafter be removed, replaced, or remain.

The present invention provides a drug delivery device obtainable by, obtained by, or directly obtained by a method of manufacture defined herein.

### Kit of Parts

The present invention provides a kit of parts comprising a drug delivery device (without the pharmaceutical composition incorporated therein), a pharmaceutical composition as defined herein (optionally contained in a separate package or container), and optionally a set of instructions with directions regarding the administration (e.g. intravenous) of the pharmaceutical composition. The user may then fill the drug delivery device with the pharmaceutical composition (which may be provided in a vial or ampoule or such like) prior to administration.

### Method of Manufacture

The present invention provides a method of manufacturing a pharmaceutical composition, preferably as defined herein. The method preferably comprises mixing together, in any particular order deemed appropriate, any relevant components required to form a composition as defined herein. The skilled person may refer to the Examples or techniques well known in the art for forming pharmaceutical compositions (especially those for injection via syringe, especially those for intravenous injection). Different embodiments will preferably require different combinations of components to be mixed, potentially in different amounts. The skilled person can readily deduce such combinations and amounts by reference to the foregoing disclosure relating to the pharmaceutical composition.

Preferably the method involves mixing together the relevant components, preferably in a diluent (e.g. water), preferably so that all of the components are (substantially or entirely) dissolved in the diluent.

The method may involve first preparing a pre-mixture (or pre-solution) of some or all components (optionally with some or all of the diluent) excluding the antibody, and the antibody may then itself (optionally with or pre-dissolved in some of the diluent) be mixed with the pre-mixture (or pre-solution) to afford the pharmaceutical composition, or a composition to which final components are then added to furnish the final pharmaceutical composition. Most preferably, the pre-mixture contains all components except for the antibody and optionally also some diluent (which may be used to pre-dissolve the antibody), preferably so that the antibody is added to a mixture which offers optimal stabilisation of the antibody. Preferably the aforementioned pre-mixture is prepared with the desired pH for the final formulation.

Preferably, the method involves forming a buffer system, preferably a buffer system comprising a buffering agent as defined herein. The buffer system may preferably have a pH as defined herein in relation to the buffer system per se. The buffer system is preferably formed in a pre-mixture prior to the addition of the antibody, though the buffer system may optionally be formed with the antibody present. The buffer system may be formed through simply mixing the buffering agent (supplied ready-made) with its acid/base conjugate (preferably in appropriate relative quantities to provide the desired pH—this can be determined by the skilled person either theoretically or experimentally). In the case of an acetate buffer system, this means mixing sodium acetate with acetic acid. Alternatively, the buffer system may be formed through adding a strong acid (e.g. HCl) to the buffering agent (e.g. sodium acetate) in order to form in situ the acid/base conjugate (e.g. acetic acid) (again preferably in appropriate relative quantities to provide the desired pH). Alternatively, the buffer system may be formed through adding a strong base (e.g. sodium hydroxide) to the acid/base conjugate (e.g. acetic acid) of the buffering agent (e.g. sodium acetate) in order to form in situ the buffering agent (again preferably in appropriate relative quantities to provide the desired pH). The pH of either the pre-mixture of final composition may be judiciously adjusted by adding the required quantity of strong base or strong acid, or even a quantity of buffering agent or acid/base conjugate. The same protocols may be used in relation to other buffer systems, such as histidine buffer systems, which may be formed using a combination of free (neutral) histidine with the imidazolium form of histidine, or using either an adjusting the pH accordingly (e.g. with base or acid as required).

In certain embodiments, the buffering agent and/or buffer system is pre-formed as a separate mixture, and the buffer system is transferred to a precursor of the composition (comprising some or all components save for the buffering agent and/or buffer system, preferably comprising the antibody) via buffer exchange (e.g. using diafiltration until the relevant concentrations or osmolality is reached). Additional excipients may be added thereafter if necessary in order to produce the final composition. The pH may be adjusted once or before all the components are present.

Any, some, or all components may be pre-dissolved or pre-mixed with a diluent prior to mixing with other components.

The final composition may be filtered, preferably to remove particulate matter. Preferably filtration is through filters sized at or below 1 m, preferably at 0.22 m. Preferably, filtration is through either PES filters or PVDF filters, preferably with 0.22 m PES filters.

It will be appreciated that pharmaceutical compositions of the invention may be provided, and preferably duly stored, as a liquid (preferably aqueous) pharmaceutical composition.

The present invention also provides a pharmaceutical composition obtainable by, obtained by, or directly obtained by the method of manufacture herein described.

### Method of Treatment

The present invention provides methods of treating a disease or medical disorder by administering a pharmaceutical composition, preferably as defined herein. Such methods preferably comprise administering, to a subject in need thereof, a therapeutically effective amount of the pharmaceutical composition. Administering may preferably comprise parenteral administration, which preferably includes any form of administration other than enteral and topical administration, usually by injection, and preferably includes intravenous delivery of a therapeutically effective amount of the pharmaceutical composition. Such method preferably comprises intravenous administration of a therapeutically effective amount of the pharmaceutical composition, preferably via intravenous injection, suitably along with other intravenous fluids (e.g. saline).

Where the pharmaceutical composition is a lyophilised pharmaceutical composition, prior to administration said composition is suitably reconstituted in water for injection or an appropriate intravenous fluid, suitably to afford an aqueous pharmaceutical composition, suitably as defined herein. The aqueous pharmaceutical composition is then suitably administered as defined herein, preferably via intravenous injection, suitably alongside other intravenous fluids (e.g. saline).

The present invention provides a pharmaceutical composition, preferably as defined herein, for use in treating a disease or medical disorder in a patient in need of such treatment. The present invention also provides a use of a pharmaceutical composition, preferably as defined herein, in the manufacture of a medicament forthe treatment of a disease or disorder.

The present invention provides a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is an EGFR-related and LGR5-related disease. The anti-EGFR/anti-LGR5 bispecific antibody preferably binds cancer stem cells expressing leucine-rich repeat-containing G-coupled receptor 5 (LGR5) and epidermal growth factor receptor (EGFR).

The present invention provides a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is a proliferative disease or disorder. The proliferative disease or disorder is preferably cancer. The cancer is preferably manifest in one or more solid tumours. The cancer is preferably an EGFR ligand responsive cancer, preferably that expresses a membrane associated member of the WNT pathway. The cancer is preferably an adenocarcinoma. In particular, the cancer is selected from the group consisting of: Colorectal cancer; Pancreatic cancer; Lung cancer; Breast cancer; Liver cancer; Prostate cancer; Ovarian cancer; Cervical cancer; Endometrial cancer; Head and Neck cancer; Melanoma; Testis cancer; Urothelial cancer; Renal cancer; Stomach cancer; Carcinoid cancer; and any combination thereof. Most preferably the cancer is Colorectal cancer; Pancreatic cancer; Lung cancer; Breast cancer; Liver cancer; Prostate cancer; Ovarian cancer; Cervical cancer; Endometrial cancer; Head and Neck cancer; or Melanoma. In a particularly preferred embodiment the cancer is Colorectal cancer; Pancreatic cancer; Lung cancer; Breast cancer; or Liver cancer. In a particularly preferred embodiment the cancer is a gastrointestinal cancer or Head and Neck cancer. The disease or medical disorder in question may preferably be selected from any of those disclosed in WO2017069628A2, which is hereby incorporated by reference. The plausibility of treatments of the invention is furthermore justified by WO2017069628A2 in light of the present disclosure.

The present invention provides a method of treating a disease or medical disorder, a pharmaceutical composition for use in treating a disease or medical disorder, and a use of a pharmaceutical composition in the manufacture of a medicament for a treatment of a disease or disorder as defined herein, wherein the disease or medical disorder is preferably as defined herein, and wherein the treating or treatment involves combination therapy whereby the pharmaceutical composition is administered in combination with one or more other pharmaceutical or biopharmaceutical active(s); wherein said combination therapy involves simultaneous, sequential or separate dosing of the individual components of the treatment. In some embodiments, the additional pharmaceutical or biopharmaceutical active may be present within any of the pharmaceutical compositions as defined herein.

### List of Clauses

Clauses

1. A pharmaceutical composition comprising: a multispecific antibody, functional part or derivative thereof, comprising a first binding domain binding to EGFR and a second binding domain binding to LGR5; a buffer system comprising a histidine buffer and/or a citrate buffer; a sugar component; and a non-ionic surfactant.

2. The pharmaceutical composition according to clause 1, wherein the composition is an aqueous composition.

3. The pharmaceutical composition according to clause 1 or 2, wherein the sugar component is or comprises sucrose.

4. The pharmaceutical composition according to any of the preceding clauses, wherein the non-ionic surfactant is or comprises polysorbate 80.

5. The pharmaceutical composition according to any of the preceding clauses, wherein the buffer system comprises a histidine buffer system and the composition has a pH of 5-7, preferably 5.4-6.6, more preferably 5.6-6.4, most preferably 5.7-6.2.

6. The pharmaceutical composition according to any of the preceding clauses, wherein the buffer system comprises a citrate buffer system and the composition has a pH of 6.2-7.8, preferably 6.5-7.5, more preferably 6.8-7.2, most preferably 6.9-7.1.

7. The pharmaceutical composition according to any of the preceding clauses, wherein the antibody is present at a concentration of 0.5-150 mg/mL, preferably 1-100 mg/mL, more preferably 1 to 50 mg/mL, more preferably 1 to 30 mg/mL, more preferably 5-25 mg/mL, more preferably 15-25 mg/mL, most preferably 20 mg/mL.

8. The pharmaceutical composition according to any of the preceding clauses, wherein the sugar component is present at a concentration of 200-400 mM, preferably 250-350, more preferably 270 to 300 mM, most preferably 280 mM or 290 mM.

9. The pharmaceutical composition according to any of the preceding clauses, wherein the non-ionic surfactant is present at a concentration of 0.01-2 mg/mL, preferably 0.1-1.5 mg/mL, more preferably 0.1-0.6 mg/mL or 0.6-1.2 mg/mL, most preferably 0.2 mg/mL, 0.5 mg/mL or 1 mg/mL.

10. The pharmaceutical composition according to any of the preceding clauses, wherein the buffer system, or combination of buffer systems, is present at a concentration between 2 and 50 mM, preferably 3 to 20 mM, more preferably 4 to 12 mM, more preferably 3-7 mM or 8 to 12 mM, most preferably 5 mM or 10 mM.

11. The pharmaceutical composition according to any of the preceding clauses, wherein the molar concentration ratio of sugar component to antibody is between 250:1 and 60,000:1, preferably 500:1 and 5000:1, preferably 1460:1 and 2920:1, preferably 1825:1 and 2555:1, more preferably 1970:1 and 2190:1, most preferably about 2040:1 or 2117:1.

12. The pharmaceutical composition according to any of the preceding clauses, wherein the molar concentration ratio of non-ionic surfactant to antibody is between 1:90 and 224:1, preferably 1:50 and 20:1, preferably 1:18 and 11:1, preferably 1:2 and 8.5:1, more preferably 1:2 and 3.5:1 or 3.5:1 and 7:1, most preferably about 1.11:1, about 2.79:1, or about 5.55:1.

13. The pharmaceutical composition according to any of the preceding clauses, wherein the molar concentration ratio of buffer system (or combination of buffer systems) to antibody is between 2.9:1 and 7300:1, preferably 6:1 and 700:1, preferably 14:1 and 365:1, preferably 21:1 and 146:1, preferably 29:1 and 88:1, more preferably 21:1 and 51:1 or 58:1 and 88:1, most preferably about 36.5:1 or 73:1.

14. The pharmaceutical composition according to any of the preceding clauses, wherein the buffer system consists of a citrate or histidine buffer system.

15. The pharmaceutical composition according to any of the preceding clauses, wherein the composition comprises only a single buffer system.

16. The pharmaceutical composition according to any of the preceding clauses, wherein the composition is free of trehalose.

17. The pharmaceutical composition according to any of the preceding clauses, wherein the composition is free of polysorbate 20.

18. The pharmaceutical composition according to any of the preceding clauses, wherein the composition is free of non-buffering salt tonicifiers, most preferably free of sodium chloride.

19. The pharmaceutical composition according to any of the preceding clauses, wherein the composition is free of antioxidants.

20. The pharmaceutical composition according to any of the preceding clauses, wherein the composition is free of chelators.

21. The pharmaceutical composition according to any of the preceding clauses, wherein the antibody is a bispecific antibody.

22. The pharmaceutical composition according to any of the preceding clauses, wherein the antibody comprises an EGFR binding domain comprising a heavy chain variable region (VH) that comprises the sequence of the VH chain of MF3370, MF3755, MF4280 or MF4289 as depicted in FIG. 1; or an EGFR binding domain comprising a heavy chain variable region (VH) that comprises the sequence of the VH chain of MF3370, MF3755, MF4280 or MF4289 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

23. The pharmaceutical composition according to any of the preceding clauses, wherein the antibody comprises an EGFR binding domain comprising a CDR1, CDR2 and/or CDR3 amino acid sequence of the VH chain of MF3370, MF3755, MF4280 or MF4289 as depicted in FIG. 1; or an EGFR binding domain comprising a CDR1, CDR2 and/or CDR3 amino acid sequence of the VH chain of MF3370, MF3755, MF4280 or MF4289 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

24. The pharmaceutical composition according to any of the preceding clauses, wherein the antibody comprises an LGR5 binding domain comprising a heavy chain variable region that comprises the sequence of the VH chain of MF5790, MF5803, MF5805, MF5808, MF5809, MF5814, MF5816, MF5817 or MF5818 as depicted in FIG. 2; or the amino acid sequence of the VH chain of MF5790, MF5803, MF5805, MF5808, MF5809, MF5814, MF5816, MF5817 or MF5818 depicted in FIG. 2 having at most 15, preferably 1, 2, 3, 4, 5, 6, 7, 8, 9 or 10 and preferably having 1, 2, 3, 4 or 5 amino acid insertions, deletions, substitutions or a combination thereof, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

25. The pharmaceutical composition according to any of the preceding clauses, wherein the antibody comprises a heavy chain sequence corresponding to any sequence of FIG. 5a, 5b, 5c, 5d, 5e or 5f or a variant thereof having at least 85% sequence identity to any of the sequences of FIGS. 5a, 5b, 5c, 5d, 5e or 5f, preferably having at least 88% sequence identity to any of the sequences of FIGS. 5a, 5b, 5c, 5d, 5e or 5f, more preferably at least 92%, at least 93%, at least 94% or at least 95%, wherein the antibody preferably further comprises a variable light chain (VL) as depicted in FIG. 3 and constant light chain (CL) as depicted in FIG. 3.

26. The pharmaceutical composition according to any of the preceding clauses, wherein the antibody comprises a heavy chain sequence corresponding to any sequence of FIG. 5a, 5b, 5c, 5d, 5e or 5f or a heavy chain sequence corresponding to any sequence of FIG. 5a, 5b, 5c, 5d, 5e or 5f having at most 15, preferably 1, 2, 3, 4, 5, 6, 7, 8, 9 or 10 and preferably having 1, 2, 3, 4 or 5 amino acid insertions, deletions, substitutions or a combination thereof.

27. The pharmaceutical composition according to any of the preceding clauses, wherein the antibody comprises a binding domain comprising a light chain variable region (VL) that comprises the sequence of the VL chain as depicted in FIG. 3; or a binding domain comprising a light chain variable region (VL) that comprises the sequence of the VL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

28. The pharmaceutical composition according to any of the preceding clauses, wherein the antibody comprises a binding domain comprising a CDR1, CDR2 and/or CDR3 amino acid sequence of the VL chain as depicted in FIG. 3; or a binding domain comprising a CDR1, CDR2 and/or CDR3 amino acid sequence of the VL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

29. The pharmaceutical composition according to any of the preceding clauses, wherein the antibody comprises a light chain constant region (CL) that comprises the sequence of the CL chain as depicted in FIG. 3; or a light chain constant region (CL) that comprises the sequence of the CL chain as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof.

30. The pharmaceutical composition according to any of the preceding clauses, wherein the antibody comprises the VH amino acid sequence of MF3370 as depicted in FIG. 1 and/or the VH amino acid sequence of MF5790 as depicted in FIG. 2 and the VL amino acid sequence as depicted in FIG. 3, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

31. The pharmaceutical composition according to any of the preceding clauses, wherein the antibody comprises the VH amino acid sequence of MF3755 as depicted in FIG. 1 and/or the VH amino acid sequence of MF5816 as depicted in FIG. 2 and the VL amino acid sequence as depicted in FIG. 3, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

32. The pharmaceutical composition according to any of the preceding clauses, wherein the antibody comprises the VH amino acid sequence of MF4280 as depicted in FIG. 1 and/or the VH amino acid sequence of MF5809 as depicted in FIG. 2 and the VL amino acid sequence as depicted in FIG. 3, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

33. The pharmaceutical composition according to any of the preceding clauses, wherein the antibody comprises the VH amino acid sequence of MF4289 as depicted in FIG. 1 and/or the VH amino acid sequence of MF5818 as depicted in FIG. 2 and the VL amino acid sequence as depicted in FIG. 3, wherein the antibody preferably further comprises a variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

34. The pharmaceutical composition according to any of the preceding clauses, wherein the antibody comprises EGFR variable heavy chain MF3755, MF3370, MF4280 or MF4289 as depicted in FIG. 1, LGR5 variable heavy chain MF5790, MF5809, MF5816 or MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

35. The pharmaceutical composition according to any of the preceding clauses, wherein the composition is an aqueous composition comprising: 1-30 mg/mL bispecific antibody, functional part or derivative thereof, comprising a first binding domain binding to EGFR and a second binding domain binding to LGR5; 3-20 mM buffer system comprising a histidine buffer and/or a citrate buffer; 200-400 mM sugar and/or sugar alcohol component; and 0.01-2 mg/mL non-ionic surfactant; wherein the composition has a pH between 5.4 and 7.4. Preferably, the composition has a pH between 5.4 and 6.4 and comprises a histidine buffer or the composition has a pH of between 6.6 and 7.4 and comprises a citrate buffer.

36. The pharmaceutical composition according to any of the preceding clauses, wherein the composition is an aqueous composition comprising: 15-25 mg/mL bispecific antibody, functional part or derivative thereof, comprising a first binding domain binding to EGFR and a second binding domain binding to LGR5; 4-12 mM buffer system comprising a histidine buffer and/or a citrate buffer; 250-350 mM sucrose; and 0.1-1.5 mg/mL polysorbate 80; wherein the composition has a pH between 5.4 and 7.4. Preferably, the composition has a pH between 5.4 and 6.4 and comprises a histidine buffer or the composition has a pH of between 6.6 and 7.4 and comprises a citrate buffer.

37. The pharmaceutical composition according to any of the preceding clauses, consisting of: 20 mg/mL bispecific antibody, functional part or derivative thereof, comprising a first binding domain binding to EGFR and a second binding domain binding to LGR5; 10 mM histidine buffer system; 280 mM sucrose; 0.5 mg/mL polysorbate 80; and water; wherein the composition has a pH of 5.8-6.0.

38. The pharmaceutical composition according to any of the preceding clauses, wherein the composition comprises: a bispecific antibody, functional part or derivative thereof, comprising a first binding domain binding to EGFR and a second binding domain binding to LGR5; a buffer system comprising a histidine buffer and/or a citrate buffer, wherein the molar concentration ratio of buffer system (or combination of buffer systems) to antibody is between 21:1 and 146:1; a sugar and/or sugar alcohol component, wherein the molar concentration ratio of sugar and/or sugar alcohol component to antibody is between 1460:1 and 2920:1; and a non-ionic surfactant, wherein the molar concentration ratio of non-ionic surfactant to antibody is between 1:18 and 11:1; wherein the composition has a pH between 5.4 and 7.4. Preferably, the composition has a pH between 5.4 and 6.4 and comprises a histidine buffer or the composition has a pH of between 6.6 and 7.4 and comprises a citrate buffer.

39. The pharmaceutical composition according to any of the preceding clauses, wherein the composition comprises: a bispecific antibody, functional part or derivative thereof, comprising a first binding domain binding to EGFR and a second binding domain binding to LGR5; a buffer system comprising a histidine buffer and/or a citrate buffer, wherein the molar concentration ratio of buffer system (or combination of buffer systems) to antibody is between 29:1 and 88:1; sucrose, wherein the molar concentration ratio of sucrose to antibody is between 1825:1 and 2555:1; and polysorbate 80, wherein the molar concentration ratio of polysorbate 80 to antibody is between 1:2 and 8.5:1; wherein the composition has a pH between 5.4 and 7.4. Preferably, the composition has a pH between 5.4 and 6.4 and comprises a histidine buffer or the composition has a pH of between 6.6 and 7.4 and comprises a citrate buffer.

40. The pharmaceutical composition according to any of the preceding clauses, wherein the composition consists of: a bispecific antibody, functional part or derivative thereof, comprising a first binding domain binding to EGFR and a second binding domain binding to LGR5; histidine buffer system, wherein the molar concentration ratio of histidine buffer system to antibody is 73:1; sucrose, wherein the molar concentration ratio of sucrose to antibody is 2040:1; polysorbate 80, wherein the molar concentration ratio of polysorbate 80 to antibody is 2.79:1; and optionally water; wherein the composition has a pH of 5.8-6.0.

41. The pharmaceutical composition according to any of the preceding clauses, wherein the antibody comprises EGFR variable heavy chain MF3755 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

42. The pharmaceutical composition according to any of the preceding clauses, wherein the antibody comprises EGFR variable heavy chain MF3755 as depicted in FIG. 1, LGR5 variable heavy chain MF5809 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

43. The pharmaceutical composition according to any of the preceding clauses, wherein the antibody comprises EGFR variable heavy chain MF3755 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

44. The pharmaceutical composition according to any of the preceding clauses, wherein the antibody comprises EGFR variable heavy chain MF3755 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

45. The pharmaceutical composition according to any of the preceding clauses, wherein the antibody comprises EGFR variable heavy chain MF3370 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

46. The pharmaceutical composition according to any of the preceding clauses, wherein the antibody comprises EGFR variable heavy chain MF3370 as depicted in FIG. 1, LGR5 variable heavy chain MF5809 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

47. The pharmaceutical composition according to any of the preceding clauses, wherein the antibody comprises EGFR variable heavy chain MF3370 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

48. The pharmaceutical composition according to any of the preceding clauses, wherein the antibody comprises EGFR variable heavy chain MF3370 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

49. The pharmaceutical composition according to any of the preceding clauses, wherein the antibody comprises EGFR variable heavy chain MF4280 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

50. The pharmaceutical composition according to any of the preceding clauses, wherein the antibody comprises EGFR variable heavy chain MF4280 as depicted in FIG. 1, LGR5 variable heavy chain MF5809 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

51. The pharmaceutical composition according to any of the preceding clauses, wherein the antibody comprises EGFR variable heavy chain MF4280 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

52. The pharmaceutical composition according to any of the preceding clauses, wherein the antibody comprises EGFR variable heavy chain MF4280 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

53. The pharmaceutical composition according to any of the preceding clauses, wherein the antibody comprises EGFR variable heavy chain MF4289 as depicted in FIG. 1, LGR5 variable heavy chain MF5790 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

54. The pharmaceutical composition according to any of the preceding clauses, wherein the antibody comprises EGFR variable heavy chain MF4289 as depicted in FIG. 1, LGR5 variable heavy chain MF5809 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

55. The pharmaceutical composition according to any of the preceding clauses, wherein the antibody comprises EGFR variable heavy chain MF4289 as depicted in FIG. 1, LGR5 variable heavy chain MF5816 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

56. The pharmaceutical composition according to any of the preceding clauses, wherein the antibody comprises EGFR variable heavy chain MF4289 as depicted in FIG. 1, LGR5 variable heavy chain MF5818 as depicted in FIG. 2, variable light chain (VL) as depicted in FIG. 3, constant light chain (CL) as depicted in FIG. 3, the CH1 domain sequence as depicted in FIG. 4, the hinge sequence as depicted in FIG. 4, the CH2 domain sequence as depicted in FIG. 4, the CH3-DE domain sequence as depicted in FIG. 4, and the CH3-KK domain sequence as depicted in FIG. 4.

57. A drug delivery device comprising a pharmaceutical composition as claimed in any one of the preceding clauses.

### EXAMPLES

The present invention is further illustrated by way of the following non-limiting examples. The examples and data presented below establish viable and plausible formulation space as well as formulation spaces which are particularly advantageous in one or more respects (e.g. fragmentation under certain stressed conditions, aggregation under certain stressed conditions, etc.).

As used herein “MFXXXX” wherein X is independently a numeral 0-9, refers to a Fab comprising a variable domain wherein the VH has the amino acid sequence identified by the 4 digits depicted in FIG. 1 and FIG. 2. Unless otherwise indicated, the light chain variable region of the variable domain (VL) typically has a sequence of FIG. 3. The light chain in the examples has a VL and CL sequence as depicted in FIG. 3. Bispecific antibodies in the examples comprise, besides the herein indicated MFXXXX sequence, a constant region of a heavy chain that normally interacts with a constant region of a light chain. The VH/variable region of the heavy chains differs and typically also the CH3 region, wherein one of the heavy chains has a KK mutation of its CH3 domain (CH3-KK) and the other has the complementing DE mutation of its CH3 domain (CH3-DE) (see for reference PCT/NL2013/050294 (published as WO2013/157954) and FIG. 4. Bispecific antibodies in the examples have an Fc tail with a KK/DE CH3 heterodimerization domain, a CH2 domain and a CH1 domain as indicated in FIG. 4, a common light chain as indicated in FIG. 3 and a VH as specified by the MF number. For example a bispecific antibody indicated by MF3755×MF5816 has the above general sequences and a variable domain with a VH with the sequence of MF3755 and a variable domain with a VH with the sequence of MF5816. Suitably, bispecific antibodies comprising variable domains binding EGFR with the sequences of MF3370; MF3755; MF4280 or MF4289 and variable domains binding LGR5 with sequences of MF5790; MF5803; MF5805; MF5808; MF5809; MF5814; MF5816; MF5817; or MF5818 can be formulated according to the experiments set forth below.

### Materials and Equipment

A full length human IgG bispecific antibody comprising MF3755 as depicted in FIG. 1 and MF5816 as depicted in FIG. 2 was provided by the present Applicant in three batches for development of a stable, viable formulation, each formulated in phosphate-buffered saline (PBS), pH 7.4 containing 1 mM KH₂PO₄/5.6 mM Na₂HPO₄ and 0.9% NaCl. Batch 1 contained 2.58 mg/mL antibody, and was stored at −80° C.; Batch 2 contained 7.74 mg/mL antibody, and was stored at 2-8° C.; and Batch 3 contained 5.18 mg/mL antibody and was stored at 2-8° C. Batches 1 and 2 were used to produce the formulations of the initial high throughput screens, whilst Batch 3 was used in the later more-focussed screens. All batches were verified to be suitably consistent.

Other materials used in producing the ensuing Examples are depicted in Table A below:

TABLE A
Reagents and Materials used in example formulations

| | | Supplier code | |
|---|---|---|---|
| Name | Supplier | Batch number | Purity |
| Sodium chloride | Merck | 1.06444 | >99.5% |
| | | Lot FN1254144511 | Api EP, BP, JP, USP |
| Magnesium chloride | Sigma-Aldrich | 13152 | >99.0% Meets analytical spec. of |
| | | Lot SZBE1830V | EP, BP, FCC |
| Calcium chloride | Sigma-Aldrich | C8106 | >99.0% |
| | | Lot SLBL1812V | Meets USP testing specifications |
| Zinc chloride | Sigma-Aldrich | 14422 | >98.0% Meets analytical spec. of |
| | | Lot SZBE1070V | EP, BP, USP |
| Sodium phosphate monobasic | Sigma-Aldrich | S2554 | >98.0% |
| | | Lot BCBP1974V | Meets USP testing specifications |
| Sodium phosphate dibasic | Sigma-Aldrich | 04276 | >98.0% Meets analytical spec. of |
| | | Lot SZBE1970V | EP, BP, USP, FCC |
| Potassium phosphate monobasic | Sigma-Aldrich | 04243 | >98.0% Meets analytical spec. of |
| | | Lot SZBE1460V | EP, NF |
| Acetic acid | Sigma-Aldrich | 27225 | >99.8% Meets analytical spec. of |
| | | Lot SZBE0640V | EP, BP, USP, FCC |
| Sodium acetate | Sigma-Aldrich | S1429 | >99.0% |
| | | Lot SLBK8463V | Meets USP testing specifications |
| Citric acid | Sigma-Aldrich | 27109 | >99.5% Meets analytical spec. of |
| | | Lot SZBE1700V | EP, BP, USP |
| Sodium citrate tribasic (NaCi) | Sigma-Aldrich | 71409 | >99.8% |
| | | Lot BCBN1840V | Tested according to EP. |
| Tris (Trizma base) | Sigma-Aldrich | T6066 | >99.0% Meets EP, USP testing specifications |
| | | Lot SLBL3232V | |
| Hydrochloric acid | Sigma-Aldrich | 07102 | 36.5-38% Meets analytical spec. |
| | | Lot SZBE1710V | of EP, BP, NF |
| Succinic acid | Sigma-Aldrich | S9512 | >99.0% |
| | | Lot BCBH5921V | For cell culture |
| Sodium succinate | Sigma-Aldrich | 14170 | >99.0% (NT) |
| | | Lot BCBL7363V | |
| L-Histidine | Sigma-Aldrich | H6034 | >98.5% Meets analytical spec. of EP, USP |
| | | Lot SLBK0726V | |
| L-Histidine-HCl | Sigma-Aldrich | H5659 | >98.5% |
| | | Lot SLBJ1942V | Meets EP analytical spec. |
| Sucrose | Sigma-Aldrich | 16104 | Meets EP, BP, NF specifications |
| | | Lot SZBD0040V | |
| Lactose | Sigma-Aldrich | 17814 | Tested according to EP. |
| | | Lot BCBN7619V | |
| D-Maltose monohydrate | Sigma-Aldrich | M5895 | >98.0% |
| | | Lot SLBK8550V | For cell culture |
| D-Mannitol | Sigma-Aldrich | M8429 | >98.0% Meets EP, FCC, USP testing |
| | | Lot SLBL0926V | specifications |
| D-Sorbitol | Sigma-Aldrich | S3889 | >98.0% |
| | | Lot SLBJ4425V | For cell culture |
| Dextrose | Sigma-Aldrich | D9434 | >99.5% Meets EP, BP, JP, USP testing |
| | | Lot SLBH3471V | specifications |
| D-Trehalose dihydrate | Sigma-Aldrich | T0167 | >99.0% |
| | | Lot SLBL8319V | For cell culture |
| Glycine | Sigma-Aldrich | 15527 | >99.0% Meets analytical spec. of EP, BP, |
| | | Lot SZBE1750V | USP |
| L-Lysine-HCl | Sigma-Aldrich | L8662 | Meets EP, JP, USP testing specifications |
| | | Lot SLBH2925V | |
| L-Methionine | Sigma-Aldrich | M5308 | >99.0% Meets EP, JP, USP testing |
| | | Lot SLBK3549V | specifications |
| Proline | Sigma-Aldrich | P5607 | from non-animal source, meets EP, USP |
| | | Lot SLBF2263V | testing specifications |
| Cysteine | Sigma-Aldrich | C5360 | Pharma grade, from non-animal source |
| | | Lot CDBB2811V | |
| EDTA | Sigma-Aldrich | 27285 | Meets analytical spec. of EP, BP, USP |
| | | SZBF0130V | |
| Tween ® 80 | NOF | — | USP, NF, EP, JP |
| | | Lot 402361 | |
| Tween 20 | Thermo | 28320 | 10% solution |
| | Scientific | Lot QE2118997 | |
| Pluronic ® F68 | Sigma | P5556 | 10% solution |
| | | Lot 23K2411 | insect cell culture tested |

The following equipment and consumables were used to both produce and perform analysis in respect of the ensuing examples:

TABLE B
Equipment used in production and analysis

| | Model | Software | |
|---|---|---|---|
| Equipment name | Vendor | Measurements | Analysis |
| Water purification system | Milli-Q Reference | N/A | N/A |
| | Millipore | | |
| PH meter | SevenMulti | N/A | N/A |
| | Mettler Toledo | | |
| Balance | XS 105 | N/A | N/A |
| | Mettler Toledo | | |
| Centrifuge | Universal 320R | N/A | N/A |
| | Hettich | | |
| Oven | IL53 IL115 | N/A | N/A |
| | VWR | | |
| Laboratory Shaker | Promax 2020 | N/A | N/A |
| | Heidolph | | |
| UV Spectrophotometer | NanoDrop ND-1000 | ND-100 | Microsoft Excel ® |
| | Thermo Scientific | | |
| UV Double beam | Cary UV 300 Bio | Varian UV Scan | GRAMS A/I |
| Spectrophotometer | Agilent Technology | Application | |
| Spectrofluorometer | FluoroMax-4 | DM3000F | GRAMS A/I |
| | Jobin Yvon Horiba | | |
| Microscope | DMi8 | LAS X | N/A |
| | Leica Microsystems | | |
| Therapeomic Scanner | Epson 4870 | Epson Scan | N/A |
| | Epson | | |
| Absorbance and fluorescence | Safire² | Xfluor4Safire | Microsoft Excel ® |
| plate reader | TECAN | | |
| Liquid handler for plates | Rapidplate | RapidPlate Driver | N/A |
| | Caliper Life Science | | |

TABLE C
Consumables used in production and analysis

| Consumable | Model | Brand |
|---|---|---|
| Quartz cuvette 1 cm × 2 mm | 115F-QS | Hellma |
| Black micro-cuvette 1 cm × 2 mm | 105.250-QS | Hellma |
| 0.1 μm PVDF membrane filters 47 mm | Durapore VVLP04700 | Millipore |
| 0.22 μm PVDF membrane filters 47 mm | Durapore GVWP04700 | Millipore |
| 0.22 μm PVDF syringe filters 13 mm | Millex SLGV013SL | Millipore |
| Microscope slides | Fastread 102 counting slide | Immune Systems |
| Glass vials | 1.5 ml vial, 32 × 11.6 mm | BGB Analytik |
| Glass inserts | 300 μl Conical Glass Insert, 31 × 6 mm, | BGB Analytik |
| Septa | Septum for screw caps, PTFE/red silicone | Agilent |
| Caps without septa | Cap, screw, blue, open top | Agilent |
| Caps with septa | 9 mm screw caps, white silicone/red PTFE | Interchim |
| Centrifugal Filter Unit, 10 kDa, 15 mL | Amicon ® Ultra-15 10 kDa | Millipore |
| Dialysis cassette 30-70 mL (3.5 kDa) | Slide-A-Lyzer 87726 | ThermoFisher |
| pH probe (micro-electrode) | InLab Ultra-Micro | Mettler Toledo |
| 2R Fiolax vials | 2R Fiolax clear, Blow back, item 58112 | — |
| Stoppers for 2R vials | 2 × 13 mm Gusto S2 F451 RS D777-1 | — |
| Alucaps for 2R vials | Kombiba Alu-Ku violet 5008, 13 mm | — |
| 18 Gauge needles | BD Microlance 3 | BD |
| Centrifugal Filter Unit, 10 kDa, 15 mL | Amicon ® Ultra-15 10 kDa | Millipore |
| Dialysis cassette 3-12 mL (3.5 kDa) | Slide-A-Lyzer 66110 | ThermoFisher |
| Dialysis cassette 12-30 mL (3.5 kDa) | Slide-A-Lyzer 66130 | ThermoFisher |
| Half Area 96-well plates | Costar 3679 | Corning |
| Transparent adhesive sealer | VIEWsealTM | Greiner Bio-One |
| pH probe (micro-electrode) | InLab Ultra-Micro | Mettler Toledo |
| 15 ml centrifuge tubes | 15 mL Falcon polypropylene tubes | BD |
| 50 ml centrifuge tubes | 50 mL polypropylene tubes | Corning |
| 1.5 ml Microtubes | Safe-Lock 1.5 mL tubes | Eppendorf |
| 0.5 ml Microtubes | 0.5 mL tubes | Plastibrand |
| 1 ml tips | GPS-L1000 | Mettler Toledo |
| 200 μl tips | GPS-L250 | Mettler Toledo |
| 20 μl tips | GPS-L10 | Mettler Toledo |
| 100 μl tips for robot | Tempo-100 | Zymark |
| Consumable | Model | Brand |
| Black micro-cuvette 3 mm × 3 mm | 105.251-QS | Hellma |
| Microscope slides | Quick Read counting slide | Milian |

### Analytical Protocols

Properties of the bispecific antibody binding both EGFR and LGR5 were characterized by different methods, including those in Table D below:

TABLE D
Methods used to investigate properties of protein formulations

| Methods | Properties |
|---|---|
| UV-Vis absorbance | Determination of protein concentration. Qualitative information on aggregation; detection of |
| | particles with sizes between 100-200 nm and micrometers |
| 90° light scatter | Sensitive in detecting changes in protein aggregation. Qualitative for size determination; can |
| | detect small changes in aggregation (e.g., 2%-4% formation of antibody dimers) |
| Intrinsic fluorescence | Information on Trp, Tyr environments, hydrophobicity and rigidity: |
| emission (Trp, Tyr) | When fluorescence intensity increases: Trp, Tyr in more hydrophobic, rigid local environment |
| | Shift of emission maximum to shorter wavelengths (blue shift) indicative of rigid environment; |
| | shift of emission maximum to longer wavelengths (red shift) indicative of more mobile |
| | environments for Trp, Tyr, suggesting unfolded structure, denaturation. Provides information |
| | on aggregation and chemical degradation |
| 1,8-ANS fluorescence | 1,8-ANS binds through electrostatic interactions to cationic groups through ion pair formation; |
| spectroscopy | can detect changes in protein structure and conformation. Provides information on |
| | aggregation and chemical degradation |

### Cuvettes

To measure excitation and emission using a 90° angle, two types of cuvettes were used: a transparent cuvette of 450 μl (Hellma 115F-QS) and a black cuvette of 150 μl (Hellma 105-250-QS), both with asymmetrical pathlengths of 1 cm×2 mm. The cuvette was used in the l/s position which means that the large face is at the excitation side and the small face at the emission side.

### UV-VIS Spectroscopy Using the Cary-UV Spectrophotometer

UV-Vis absorbance spectra were recorded at 25° C. (Peltier control) with a double beam, two monochromators Cary 300 Bio spectrophotometer from Varian. Before any measurement, the baseline was recorded against air. Absorbance was monitored using a 450 μl transparent quartz cuvette (Hellma 115F-QS) with a pathlength of 1 cm. For high concentration samples, the signal was saturated at 280 nm; thus absorbance was shown between 310 and 400 nm.

The region between 320 nm and 400 nm provides information on the aggregation state of the samples [e.g., Eckhardt, 1994; Mulinacci, 2011b; and Peters, 2013].

### UV-VIS Spectroscopy Using the NanoDrop Spectrophotometer

A NanoDrop® ND-1000 spectrophotometer (Witec AG, Switzerland) Foutl Verwijzingsbron niet gevonden. was used to determine antibody concentration in solutions at high concentrations. Absorbance measurements were performed using pathlengths of 0.1 mm and 1 mm in a 3 μl drop of solution. Absorbance spectra were recorded at room temperature with the software module Protein A280 and with air as blank. Normalization at 340 nm was used. A minimum of five drops were measured in order to have reproducible data.

To measure absorbance with the NanoDrop, the UV-Vis absorbance spectrum is measured from the top through to the bottom in a 2-3 μl drop.

### 90° Light-Scattering Spectroscopy

Measurements were performed using a FluoroMax-4 spectrofluorometer (HORIBA Jobin Yvon GmbH, Germany) by running a synchronous scan (λₑₘ=λₑₓ) between 400 nm and 750 nm. 90° light-scattering spectra were monitored at 25° C. in a 1 cm×2 mm, 150 μl black cuvette in position l/s (Hellma 105-250-QS) with an integration time of 0.01 s. Excitation and emission slits of 1 nm and 1.5 nm, respectively, were used. All spectra were recorded using an increment of 1 nm and with the mode S/R: the sample signal is corrected for the signal of the reference (fluorescence lamp). A refrigerated circulator bath AC200 A25 (Thermo Scientific) was used to thermostat the FluoroMax-4 Spectrofluorometer at 25° C.

90° light-scattering spectral method was developed to study protein aggregation in formulations [e.g. Capelle, 2005; Demeule, 2007a and b; Mulinacci, 2011a and 2013; Luca, 2010; Patois, 2011 and 2012; Peters, 2013]. The scattering of light in solution is dependent on different factors, such as protein concentration, refractive index, particle size and shape, and the wavelength of the incident light.

### 1,8-ANS Fluorescence Staining

150 μl of sample were added in a 1 cm×2 mm black cuvette (Hellma 105-250-QS); and 7.5 μl of 1 mM ANS in water were added on top of it (final concentration: 50 μM ANS). The sample was measured immediately after addition of ANS. ANS fluorescence emission was measured on the FluoroMax-4 spectrofluorometer.

1-anilinonaphthalene-8-sulfonic acid (1,8-ANS) is an uncharged small hydrophobic fluorescent probe (MW 299.34 Da) used to study both membrane surfaces and proteins. ANS is essentially non-fluorescent in water and becomes fluorescent when bound to electrostatic pockets in proteins, protein aggregates, detergent micelles, leachables such as silicon oil, or membranes and cellular components. Publications describe the use of ANS in various studies of protein aggregation [e.g. Demeule, 2009; Mulinacci, 2011a and b; Luca, 2010].

### Fluorescence Emission Spectroscopy

Fluorescence emission spectra were monitored at 25° C. using the FluoroMax-4 spectrofluorometer in a 1 cm×2 mm, 150 μl black cuvette in position l/s (Hellma 105-250-QS). All spectra were recorded using an increment of 1 nm with the mode S/R. A refrigerated circulator bath was used to thermostat the FluoroMax-4 Spectrofluorometer at 25° C.

Intrinsic fluorescence emission parameters: excitation at 280 nm; emission between 290 nm and 450 nm; integration time of 0.05 s. Excitation slit of 1 nm and emission slit of 1.5 nm.

1,8-ANS fluorescence emission parameters: excitation at 370 nm; emission between 390 nm and 650 nm; integration time of 0.05 s. Excitation slit of 2 nm and emission slit of 4 nm.

The fluorescence emission of tryptophan (Trp) and tyrosine (Tyr) gives information on the local environment of these fluorophores [e.g. Capelle, 2005; Demeule, 2007a and b and 2009; Mulinacci, 2011a and b; Luca, 2010; Patois, 2011; Peters, 2013]. Changes or differences in hydrophobicity and/or rigidity are measured. Typically, a more hydrophobic and rigid environment leads to an increase in the fluorescence intensity and a shift to shorter wavelengths (blue shift) of the emission maximum.

### Stress Testing Protocols

The antibody formulations of the ensuing Examples were analyzed (e.g. using the aforesaid protocols, e.g. to assess aggregation) after exposing said formulations to certain stressed conditions.

### Initial Condition (Time 0)

All samples were prepared (on the same day for particular formulation screens), and the Time 0 condition corresponds to be kept for 1 day at 2-8° C. after such preparation.

### Thermal Stressing

To thermally stress a sample, a glass vial was filled with a suitably amount of the sample (generally 200 μL or 1 mL) and then incubated for sufficient time at elevated temperatures as indicated per investigated plate (e.g. 2 days at 50° C. before being subsequently stored for one day at 2-8° C. before measurement.

### Freeze-Thaw Stressing

To stress samples by freezing and thawing, a cryogenic tube was filled with sufficient sample (generally 200 μL or 1 mL) and the tube was then frozen at −80° C. for a minimum of 1 hour before being thawed at room temperature for one hour. This cycle of freezing and thawing was repeated 2 more times, to give a total of 3 freeze-thaw cycles. After all 3 freeze-thaw cycles, the sample was stored for one day at 2-8° C. before further measurements.

General Overview of High Throughput Screen of Formulations with 1 mg/ml Antibody

The first high throughput screenings were performed upon various different antibody formulations, each with 1 mg/ml bispecific antibody binding both EGFR and LGR5. The screened formulations were distributed amongst several plates, as described below.

Solutions with antibody were dialyzed using Slide-A-Lyzer Dialysis Cassettes with a molecular-weight cut-off of 3.5 kDa. The cassettes were immersed in 4.5 liters of milliQ water at 2-8° C., under gentle stirring overnight.

Due to the dialysis process (expansion of dialysis cassette), a dilution of antibody occurred. The concentration of the sample was determined by measuring the UV-Vis absorbance by a NanoDrop spectrophotometer (Table 1).

To determine the concentration of antibody solutions, absorbance spectra were monitored using a NanoDrop® ND-1000 spectrophotometer. Measurements were performed using pathlengths of 0.1 mm and 1 mm in a 3 μl drop of solution that permits the measurement of concentrated solutions. Absorbance spectra were recorded at room temperature with the software module Protein A280 and with air as blank. Normalization at 340 nm was used. A minimum of three drops were measured in order to have reproducible data.

TABLE 1
Absorbance at 280 nm of antibody solutions. The concentration
of the sample was calculated from a known concentration
of a batch of the antibody at 2.58 mg/ml.

| | | Mean | Mean |
|---|---|---|---|
| Antibody | Absorbance at 280 nm | absorbance | concentration |
| in PBS pH 7.4 | 3.60-3.60-3.54-3.55-3.59 | 3.58 ± 0.03 | 2.58 mg/ml |
| dialyzed in | 2.64-2.62-2.62-2.61-2.61 | 2.62 ± 0.03 | 1.89 mg/ml |
| water | | | |

For the initial set of plates, a final concentration of 1 mg/ml antibody was used in all formulations as indicated using proper dilutions as needed. No concentration step was needed regarding the concentration of the starting material (2.58 mg/ml) and its concentration after dialysis (always around 1.9 mg/ml). After the dialysis step, the samples containing antibody were used within one day.

For High-Throughput Formulation (HTF) experiments, the antibody solutions in water were diluted in different formulations. Concentrated excipients stock solutions were used to prepare the different formulations (Table 2)

TABLE 2
Concentrated excipients stock solutions.

| Salts | Sugars | Detergents (or surfactant) | Others |
|---|---|---|---|
| 1M NaCl | 1M Trehalose (Tre) | 2% Tween ® 20 (T20 or PS20) | 1M Proline (Pro) |
| 200 mM MgCl₂ | 1M Sucrose (Suc) | 2% Tween ® 80 (T80 or PS80) | 1M Glycine (Gly) |
| 200 mM CaCl₂ | 1M Mannitol (Man) | 10% F68 (F68) (also known as | 200 mM Lysine (Lys) |
| 200 mM ZnCl₂ | 1M Sorbitol (Sor) | Poloxamer 188 or P188) | 100 mM Cysteine (Cys) |
| | 1M Dextrose (Dex) | | 100 mM Methionine (Met) |
| | 1M Maltose (Mal) | | 100 mM EDTA |
| | 600 mM Lactose (Lac) | | |

Different buffer were also prepared as following: different volumes of acid and base were mixed until having the desired pH.
200 mM Histidine buffer (Hist): mixing of 200 mM Histidine with 200 mM Histidine-HCl
200 mM Na-Citrate buffer (NaC: mixing of 200 mM citric acid with 200 mM sodium citrate
200 mM Na-Acetate buffer (NaA): mixing of 200 mM acetic acid with 200 mM sodium acetate
200 mM Na-Phosphate buffer (NaP): mixing of 200 mM NaH₂PO₄ with 200 mM Na₂HPO₄
200 mM Na/K-Phosphate buffer (NaKP): mixing of 200 mM KH₂PO₄ with 200 mM Na₂HPO₄
200 mM Tris buffer (Tris): 200 mM Trizma base with some drops of 2M HCl
200 mM Na-Succinate buffer (NaS): mixing of 200 mM Succinic acid with 200 mM sodium succinate
200 mM Na-Citrate-Phosphate (CitP) buffer: mixing of 200 mM Citric acid with 400 mM Na₂HPO₄
   Preparation and Analysis of Plates Used at 1 mg/ml Antibody

100 μl of each antibody formulation were dispensed in half-area UV-transparent 96-well Costar Corning plates and measured just after preparation of the formulations (Time 0). The plates were sealed with VIEWseal™ Transparent adhesive sealers before freezing/thawing or temperature stress.

The center 60 wells of a 96-well plate were used to test antibody formulations; experiments were in duplicate. As controls in each plate starting antibody material was used (2.58 mg/ml) and either diluted in 6 mM Sodium-Potassium phosphate buffer pH 7.5 with 154 mM NaCl (PBS) or antibody dialyzed in water, as indicated where appropriate as NaKP or water.

Antibody formulations were characterized before and after 3 freeze-thaw cycles by Therapeomic scanner (Epson 4870), UV-Vis absorbance and intrinsic fluorescence emission. After 3 freeze-thaw cycles, plates were incubated at 40° C. or 50° C. as indicated and analyzed with the same methods. Where indicated, plates were prepared in triplicate and measured by Therapeomic scanner, UV-Vis absorbance intrinsic and 1,8-ANS fluorescence emission: the first plate was measured at Time 0, the second after 3 freeze-thaw cycles and the third plate was measured after 2 days at 50° C.

The analysis of each plate before and after stress generates a large amount of data. From the solutions in each well, UV and fluorescence spectra were recorded. From these spectra, numeric values were chosen to determine and compare antibody stability in different formulations:
absorbance 350 nm (UV-350 nm), information on the protein aggregation state
maximum Trp fluorescence intensity (Emission Max)
emission wavelength at maximum intrinsic fluorescence intensity (λₘₐₓ)
maximum 1,8-ANS fluorescence intensity
emission wavelength at maximum 1,8-ANS fluorescence intensity

Result as obtained for absorbance and fluorescence emission, visual aspect (scanner) of antibody formulations before (T0) and after three freeze-thaw cycles (3FT) are mentioned below.

To prepare the different formulations, concentrated buffer solutions were used. The pH of each formulation after preparation and mixing with the antibody was measured in the 96-well plate and are indicated in the below tables. At 1 mg/ml minor differences were observed between the pH of the starting stock solution and the final pH. No pH measurements were performed where 1,8-ANS was added to the wells due to its effect on the pH.

Analysis of the Safire² Excel® worksheets:

To facilitate data analysis, different values were extracted from the absorbance and fluorescence spectra:
Turbidity is the absorbance at 350 nm: gives information on the aggregation state of the sample;
Maximum intrinsic emission intensity: gives information on Trp, Tyr environments, hydrophobicity and rigidity; when fluorescence intensity increase: Trp, Tyr in more hydrophobic, rigid local environment;
Emission wavelength at maximum intrinsic fluorescence intensity: shift of emission maximum to shorter wavelengths (blue shift) indicative of rigid environment; shift of emission maximum to longer wavelengths (red shift), more mobile environments for Trp, Tyr, indicative of unfolded structure, denaturation;
Maximum 1,8-ANS emission intensity and wavelength: can detect changes in protein structure and conformation.

### Formula:

For all formulations, duplicates measurements (2 wells) were performed; the average was calculated and displayed in summary tables. The average calculated was the arithmetic mean:

$$\overline{x} = \frac{1}{n} {\sum }_{i = 1}^{n} {x}_{i} .$$

For absorbance at 350 nm, intrinsic fluorescence intensity and 1,8-ANS fluorescence intensity a percentage of change was calculated as following:

%=100*(Xₛₜᵣₑₛₛ−X_{T0})/X_{T0}

X representing absorbance or fluorescence intensity before (T0) or after stress.

For intrinsic and 1,8-ANS emission wavelength at maximum fluorescence intensity (λₘₐₓ), the shift before and after stress was calculated:

Δλ=λₘₐₓ (after stress)−λₘₐₓ (T0)

### Change Factor:

To aid in selecting stable formulations where necessary, the Change Factor (CF) was introduced. Stable formulations had a small CF (small changes after thermal stress) following calculations from UV absorbance, intrinsic fluorescence emission and 1,8-ANS fluorescence emission changes after thermal stress:

CF=δAbs+δInt.Fluo+δANS.Fluo

| | |
|---|---|
| δAbs = \|% change Abs_{350 nm}\| | represents 80% of total CF |
| δInt. Fluo = \|% change Int. Fluo | represents 10% of total CF |
| emission\|/10 | |
| δANS = \|% change 1,8-ANS\|/10 | represents 10% of total CF |

The change factor was defined by weighting the importance of the different measurements; changes in absorbance at 350 nm are more significant than changes in fluorescence intensity; changes in 1,8-ANS fluorescence are also significant but can be very high (e.g 400%); this percentage was then divided by 10 to be at the same level than absorbance at 350 nm.

Scanner Aspect. The presence of aggregates inside each well was investigated using a Therapeomic Epson 4870 scanner and visually inspected for the presence of aggregates formed. Samples were divided into three groups based on the amounts of aggregates with amounts increasing from group 1 to group 3. Clear samples (no aggregates observed) were assigned to Group 1. Cloudy samples (slightly turbid containing small aggregates) to group 2 and Aggregated samples (very turbid with strong aggregatation) to group 3. Where reported, results are presented as 1, 2 or 3 under “Scanner Aspect” in the respective tables.

### Example 1

An objective of Example 1 using Plate 1 included investigating the effect of various sugars in inhibiting aggregation of solutions of 1 mg/ml antibody in 5 mM Na-Phosphate pH 7.0.

The results after 3 freeze-thaw cycles (3-FT) are shown in Table 3 showing stability data of 1 mg/ml antibody solutions in plate 1 before (T0) and after three freeze-thaw cycles (3FT). Turbidity is the absorbance at 350 nm and % is change between T0 and 3FT.

TABLE 3
Results of Plate 1 before and after 3-FT cycles.

| | | | | | | | Turbidity | | |
|---|---|---|---|---|---|---|---|---|---|
| Antibody with | | | | | | | UV - 350 nm | | |
| Buffer | | final pH | Salt | | Sugar | | T0 | 3 FT | % |
| NaKP | 6 mM | 7.4 | NaCl | 154 mM | — | — | 0.045 | 0.066 | 47% |
| water | — | 6.9 | — | — | — | — | 0.043 | 0.074 | 72% |
| NaP | 5 mM | 7.3 | — | — | Sor | 290 mM | 0.048 | 0.088 | 83% |
| NaP | 5 mM | 7.3 | — | — | Dex | 290 mM | 0.047 | 0.097 | 106% |
| NaP | 5 mM | 7.3 | — | — | Man | 290 mM | 0.047 | 0.122 | 160% |
| NaP | 5 mM | 7.3 | — | — | Suc | 290 mM | 0.047 | 0.059 | 26% |
| NaP | 5 mM | 7.2 | — | — | Tre | 290 mM | 0.051 | 0.065 | 27% |
| NaP | 5 mM | 7.3 | — | — | Mal | 290 mM | 0.047 | 0.066 | 40% |
| NaP | 5 mM | 7.2 | — | — | Lac | 270 mM | 0.070 | 0.087 | 24% |

The results suggest that from all tested sugars, sucrose generally gave the best results, improving the stability of antibody after three freeze-thaw cycles. In this experiment, inclusion of sucrose resulted in a low change in absorbance (26%) combined with a low absorbance at 350 nm at Time 0 (0.047). Other sugars are, however, also viable candidates for further investigation, due to their small absorbance at 350 nm after stress. Also, results from samples from this plate wherein stability was assessed on the basis of measurements at 350 nm, showed that sucrose was better at preserving stability than sodium chloride when samples were subjected to three freeze-thaw cycles (data not shown).

### Example 2

Objectives of Example 2 using Plate 2 included identifying agents that provide protection from freezing-thawing in formulations with different buffers and different sugars.

The results after 3 FT cycles are shown in Table 4 providing data on the stability of 1 mg/ml antibody solutions in plate 2 before (T0) and after three freeze-thaw cycles (31FT).

TABLE 4
Results for Plate 2 after 3 FT cycles.

| Antibody with | | | | | Turbidity | | | Intrinsic fluorescense | | | | Scanner | |
|---|---|---|---|---|---|---|---|---|---|---|---|---|---|
| | | Final | Salt/ | Conc | UV - 350 nm | | | Emission max | | | λ max | aspect | |
| Buffer | mM | pH | sugar | (mM) | T0 | 3 FT | % | T0 | 3FT | % | Δ λ | T0 | 3 FT |
| water | — | 6.4 | — | — | 0.040 | 0.063 | 58% | 37183 | 42749 | 15% | −1 | 1 | 3 |
| Nal | 5 | 6.2 | Tre | 290 | 0.046 | 0.061 | 33% | 37512 | 40282 | 7% | 0 | 1 | 3 |
| NaP | 5 | 6.3 | Suc | 290 | 0.045 | 0.056 | 24% | 38094 | 41263 | 8% | 0 | 1 | 1 |
| NaP | 5 | 7 | Tre | 290 | 0.047 | 0.065 | 38% | 37765 | 40840 | 8% | 1 | 1 | 2 |
| NaP | 5 | 7.1 | Suc | 290 | 0.044 | 0.059 | 34% | 37699 | 40275 | 7% | 1 | 1 | 2 |
| NaP | 5 | 7.3 | Tre | 290 | 0.049 | 0.068 | 39% | 37183 | 39922 | 7% | 1 | 1 | 2 |
| NaP | 5 | 7.6 | Suc | 290 | 0.045 | 0.059 | 31% | 37196 | 40080 | 8% | 0 | 1 | 2 |
| NaC | 5 | 4.5 | Tre | 290 | 0.044 | 0.053 | 20% | 34124 | 37075 | 9% | 0 | 1 | 1 |
| NaC | 5 | 4.4 | Suc | 290 | 0.045 | 0.051 | 13% | 33696 | 36430 | 8% | −1 | 1 | 1 |
| NaC | 5 | 5.5 | Tre | 290 | 0.047 | 0.06 | 28% | 34333 | 37359 | 9% | −1 | 1 | 2 |
| NaC | 5 | 5.6 | Suc | 290 | 0.047 | 0.064 | 36% | 36595 | 39769 | 9% | −3 | 1 | 3 |
| NaA | 5 | 4.2 | Tre | 290 | 0.045 | 0.056 | 24% | 34666 | 37883 | 9% | 0 | 1 | 1 |
| NaA | 5 | 4.2 | Suc | 290 | 0.044 | 0.054 | 23% | 35152 | 38549 | 10% | −1 | 1 | 1 |
| NaA | 5 | 5.1 | Tre | 290 | 0.044 | 0.056 | 27% | 35657 | 39073 | 10% | 1 | 1 | 1 |
| NaA | 5 | 5.1 | Suc | 290 | 0.044 | 0.054 | 23% | 35474 | 38626 | 9% | 0 | 1 | 1 |
| Hist | 5 | 5.8 | Tre | 290 | 0.047 | 0.06 | 28% | 35440 | 38596 | 9% | 2 | 1 | 1 |
| Hist | 5 | 5.9 | Suc | 290 | 0.045 | 0.057 | 27% | 35453 | 38739 | 9% | 0 | 1 | 1 |
| Hist | 5 | 6.5 | Tre | 290 | 0.05 | 0.066 | 32% | 34699 | 36455 | 5% | −1 | 1 | 2 |
| Hist | 5 | 6.8 | Suc | 290 | 0.05 | 0.058 | 16% | 34936 | 38026 | 9% | −1 | 1 | 1 |
| NaS | 5 | 4.1 | Tre | 290 | 0.045 | 0.058 | 29% | 31551 | 34233 | 9% | −1 | 1 | 1 |
| NaS | 5 | 4.1 | Suc | 290 | 0.041 | 0.051 | 24% | 33052 | 35602 | 8% | −2 | 1 | 1 |
| NaS | 5 | 5.1 | Tre | 290 | 0.045 | 0.055 | 22% | 34441 | 36479 | 6% | −1 | 1 | 1 |
| NaS | 5 | 5.2 | Suc | 290 | 0.044 | 0.053 | 20% | 34566 | 37270 | 8% | 0 | 1 | 1 |
| NaS | 5 | 6.1 | Tre | 290 | 0.048 | 0.059 | 23% | 35094 | 37462 | 7% | 0 | 1 | 2 |
| NaS | 5 | 6.2 | Suc | 290 | 0.044 | 0.054 | 23% | 34888 | 37492 | 7% | 2 | 1 | 1 |
| Tris | 5 | 6.8 | Tre | 290 | 0.05 | 0.064 | 28% | 34032 | 36180 | 6% | 1 | 1 | 2 |
| Tris | 5 | 7.1 | Suc | 290 | 0.05 | 0.061 | 22% | 33943 | 36158 | 7% | −1 | 1 | 3 |
| Tris | 5 | 7.8 | Tre | 290 | 0.054 | 0.076 | 41% | 32897 | 35181 | 7% | 1 | 1 | 3 |
| Tris | 5 | 8 | Suc | 290 | 0.05 | 0.062 | 24% | 33021 | 35240 | 7% | 1 | 1 | 3 |
| NaKP | 6 | 7.2 | NaCl | 154 | 0.043 | 0.083 | 93% | 31933 | 33640 | 5% | 0 | 1 | 3 |

Results indicate better outcomes with sucrose over trehalose in multiple different buffer systems under various tested conditions. Also in this experiment, histidine-, acetate- and succinate-based buffer systems provide good results. Higher pHs, such as those exemplified with Tris and sodium phosphate buffers, seem less favourable in terms of 3-FT and thermal stress protection. Similar results were obtained showing better results with sucrose over trehalose and results with sucrose at pH 5.9 outperforming sucrose-containing samples of higher pH in histidine buffers, after this plate was subjected to stressing conditions at 4000, following the three freeze-thaw cycles, and further analysis after a 3 day and after an 11 day incubation period (data not shown).

### Example 3

Objectives of Example 3 using Plate 3 included to study the influence of detergents, also referred to herein as surfactants, in formulations with antibody containing, 290 mM sucrose, to study a new thermal stress condition to investigate stability of the antibody after an incubation of 1 day at 5000 and to study the influence of adding various amino acids (e.g. proline, lysine, glycine, cysteine and methionine) on formulations containing sucrose (data not shown).

The results after 3 FT cycles are shown in Table 5 showing data on the stability of 1 mg/ml antibody solutions in plate 3 before (T0) and after three freeze-thaw cycles (3FT).

TABLE 5
Results from Plate 3 after 3 cycles.

| Antibody in the presence of | | | | | | | | | | | | | |
|---|---|---|---|---|---|---|---|---|---|---|---|---|---|
| 290 mM sucrose* with | | | | | Turbidity | | | Intrinsic fluorescense | | | | Scanner | |
| | | Final | Deter- | Conc | UV - 350 nm | | | Emission max | | | λ max | aspect | |
| Buffer | mM | pH | gent | (%) | T0 | 3FT | % | T0 | 3 FT | % | Δ λ | T0 | 3 FT |
| water | — | 6.6 | — | — | 0.044 | 0.063 | 43% | 36467 | 43831 | 20% | 1 | 1 | 3 |
| NaC | 5 | 5.1 | — | — | 0.046 | 0.056 | 22% | 35735 | 40851 | 14% | −1 | 1 | 2 |
| NaC | 5 | 5.1 | T20 | 0.02 | 0.051 | 0.055 | 8% | 36403 | 41408 | 14% | −1 | 1 | 1 |
| NaC | 5 | 5.1 | T80 | 0.02 | 0.046 | 0.048 | 4% | 36441 | 41875 | 15% | −1 | 1 | 1 |
| NaC | 5 | 5.1 | F68 | 0.10 | 0.045 | 0.048 | 7% | 35639 | 41028 | 15% | 0 | 1 | 1 |
| NaA | 5 | 4.7 | — | — | 0.045 | 0.056 | 24% | 34643 | 39316 | 13% | 1 | 1 | 1 |
| NaA | 5 | 4.7 | T20 | 0.02 | 0.052 | 0.053 | 2% | 35599 | 40269 | 13% | 0 | 1 | 1 |
| NaA | 5 | 4.6 | T80 | 0.02 | 0.051 | 0.054 | 6% | 35336 | 39763 | 13% | 1 | 1 | 1 |
| NaA | 5 | 4.3 | F68 | 0.10 | 0.049 | 0.052 | 6% | 35019 | 39836 | 14% | 1 | 1 | 1 |
| NaS | 5 | 4.7 | — | — | 0.046 | 0.057 | 24% | 32325 | 36629 | 13% | 0 | 1 | 1 |
| NaS | 5 | 4.7 | T20 | 0.02 | 0.052 | 0.049 | −6% | 33445 | 37680 | 13% | 0 | 1 | 1 |
| NaS | 5 | 4.7 | T80 | 0.02 | 0.047 | 0.049 | 4% | 33596 | 37601 | 12% | −1 | 1 | 1 |
| NaS | 5 | 4.4 | F68 | 0.10 | 0.047 | 0.048 | 2% | 32867 | 36907 | 12% | 0 | 1 | 1 |
| Hist | 5 | 5.5 | — | — | 0.047 | 0.051 | 9% | 32535 | 36031 | 11% | 0 | 1 | 1 |

After the 3 freeze-thaw cycles, Plate 3 was incubated for 1 day at 5000. The results after incubation for 1 day at 5000 are shown in Table 6. Table 6 shows a data overview of the stability of 1 mg/ml antibody solutions in plate 3 after three freeze-thaw cycles (3FT) and 1 day at 5000 (1 d50′C). *Sample indicated as water does not contain any sucrose.

TABLE 6
results from Plate 3 after three freeze-thaw cycles and 1 day incubation at 50° C.

| Antibody in the presence of | | | | | | Turbidity | | | Intrinsic fluorescense | | | | Scanner | |
|---|---|---|---|---|---|---|---|---|---|---|---|---|---|---|
| 290 mM sucrose* with | | | | | | UV - 350 nm | | | Emission max | | | | aspect | |
| | | | Final | Deter- | Conc | | 1 d | | | 1 d | | λ max | | 1 d |
| Buffer | mM | pH | pH | gent | (%) | 3 FT | 50° C. | % | 3 FT | 50° C. | % | Δ λ | 3 FT | 50° C. |
| water | — | — | 6.6 | — | — | 0.063 | 0.053 | −16% | 43831 | 49742 | 13% | −1 | 3 | 1 |
| NaC | 5 | 4.5 | 5.1 | — | — | 0.056 | 0.059 | 5% | 40851 | 56397 | 38% | 2 | 2 | 2 |
| NaC | 5 | 4.5 | 5.1 | T20 | 0.02% | 0.055 | 0.059 | 7% | 41408 | 57809 | 40% | 1 | 1 | 1 |
| NaC | 5 | 4.5 | 5.1 | T80 | 0.02% | 0.048 | 0.052 | 8% | 41875 | 62733 | 50% | 1 | 1 | 1 |
| NaC | 5 | 4.5 | 5.1 | F68 | 0.10% | 0.048 | 0.055 | 15% | 41028 | 62104 | 51% | 11 | 1 | 1 |
| NaA | 5 | 4.5 | 4.7 | — | — | 0.056 | 0.054 | −4% | 39316 | 46433 | 18% | −1 | 1 | 1 |
| NaA | 5 | 4.5 | 4.7 | T20 | 0.02% | 0.053 | 0.052 | −2% | 40269 | 55245 | 37% | 0 | 1 | 1 |
| NaA | 5 | 4.5 | 4.6 | T80 | 0.02% | 0.054 | 0.053 | −2% | 39763 | 48681 | 22% | 0 | 1 | 1 |
| NaA | 5 | 4.5 | 4.3 | F68 | 0.10% | 0.052 | 0.052 | 0% | 39836 | 61543 | 54% | 1 | 1 | 1 |
| NaS | 5 | 4.5 | 4.7 | — | — | 0.057 | 0.054 | −5% | 36629 | 46322 | 26% | −1 | 1 | 1 |
| NaS | 5 | 4.5 | 4.7 | T20 | 0.02% | 0.049 | 0.05 | 2% | 37680 | 52601 | 40% | 0 | 1 | 1 |
| NaS | 5 | 4.5 | 4.7 | T80 | 0.02% | 0.049 | 0.05 | 2% | 37601 | 48752 | 30% | 1 | 1 | 1 |
| NaS | 5 | 4.5 | 4.4 | F68 | 0.10% | 0.048 | 0.052 | 8% | 36907 | 63021 | 71% | 0 | 1 | 1 |
| Hist | 5 | 5.5 | 5.5 | — | — | 0.051 | 0.052 | 2% | 36031 | 42869 | 19% | 2 | 1 | 1 |

The results of Plate 3 suggest that formulations containing surfactants show more tolerance to 3-FT cycles. In respect of 3-FT, Tween® 80 and Pluronic® F68 slightly outperform Tween® 20. Histidine buffered formulations perform surprisingly well even without surfactants under both 3-FT and thermal stressing. In respect of thermal stressing, surfactants appear to improve formulation stability, though Tween® 20 and 80 slightly outperform Pluronic® F68. Taken together, the results indicate Tween® 80 to give the best results in this experiment. Although no improvements were reported by adding proline, lysine, glycine, cysteine and methionine on tested aspects of formulations in this experiment (data not shown), they do not undermine formulation viability. *Sample indicated as water does not contain any sucrose.

### Example 4

Objectives of Example 4 using Plate 4 included to find formulations with different buffer systems that contain about 290 mM Sucrose and 0.02% Tween 80 that could protect against three freeze-thaw cycles and incubation at 500 And also to study the influence of salts such as CaCl₂, MgCl₂, ZnCl₂, KCl and EDTA in small amounts (data not shown).

The results after 3 FT cycles are shown in Table 7 which shows data on the stability of 1 mg/ml antibody solutions in Plate 4 before (T0) and after three freeze-thaw cycles (3FT).

TABLE 7
results from plate 4 after three freeze-thaw cycles.

| Antibody with or without | | | | | | | | | | | |
|---|---|---|---|---|---|---|---|---|---|---|---|
| T80 as detergent at 0.02% | | | | | Turbidity | | | Intrinsic fluorescense | | | |
| | | Final | Deter- | | UV - 350 nm | | | Emission max | | | λ max |
| Buffer | mM | pH | gent | Sugar (mM) | 0 FT | 3 FT | % | 0 FT | 3 FT | % | Δ λ |
| water | — | 6 | — | — | 0.041 | 0.053 | 29 | 36295 | 41041 | 13 | 0 |
| NaS | 5 | 4.1 | — | Suc (290) | 0.041 | 0.050 | 22 | 33908 | 37637 | 11 | −2 |
| NaS | 5 | 4.1 | T80 | Suc (290) | 0.044 | 0.046 | 5 | 33864 | 37369 | 10 | 1 |
| NaS | 5 | 5.7 | T80 | Suc (290) | 0.051 | 0.054 | 6 | 36594 | 40161 | 10 | −2 |
| NaS | 10 | 5.6 | T80 | Suc (280) | 0.051 | 0.053 | 4 | 37014 | 40628 | 10 | 0 |
| NaS | 20 | 5.6 | T80 | Suc (260) | 0.05 | 0.055 | 10 | 36774 | 40816 | 11 | −1 |
| CitP | 5 | 4.4 | — | Suc (290) | 0.05 | 0.057 | 14 | 34946 | 38524 | 10 | −1 |
| CitP | 5 | 4.4 | T80 | Suc (290) | 0.049 | 0.054 | 10 | 34780 | 39133 | 13 | 1 |
| CitP | 5 | 5.9 | T80 | Suc (290) | 0.051 | 0.054 | 6 | 35947 | 39815 | 11 | 0 |
| CitP | 10 | 5.9 | T80 | Suc (280) | 0.051 | 0.053 | 4 | 35564 | 39142 | 10 | −1 |
| CitP | 20 | 5.8 | T80 | Suc (260) | 0.048 | 0.051 | 6 | 34663 | 38589 | 11 | −1 |
| Hist | 5 | 5.4 | — | Suc (290) | 0.046 | 0.057 | 24 | 32958 | 36303 | 10 | 1 |
| Hist | 5 | 5.3 | T80 | Suc (290) | 0.047 | 0.050 | 6 | 34181 | 37329 | 9 | 0 |
| Hist | 10 | 5.3 | T80 | Suc (280) | 0.046 | 0.052 | 13 | 34738 | 37647 | 8 | −1 |
| Hist | 20 | 5.3 | T80 | Suc (260) | 0.047 | 0.052 | 11 | 33787 | 36034 | 7 | −1 |

In respect of 3-FT stressing, the results of Plate 4 suggest that addition of Tween® 80 provides an improvement in the stability of formulations with good results being achieved in a histidine-based buffer system. EDTA was not found to increase the stability of the tested antibody, neither did the addition of salts (like KCl, CaCl₂, MgCl₂ and ZnCl₂) provide an improvement in stability after 3FT and incubation at 5000 for 1 day, compared to a histidine-buffer system with T80 and sucrose at 290 nm (data not shown). However, formulation viability was not undermined by any of these extra components. Scanner aspect data is not presented since this criterion was not a differentiating factor in this experiment.

### Example 5

Plate 5 as used for Example 5 was prepared in a sufficiently large volume to allow division thereof over three plates. For triplicate measurements of turbidity, intrinsic and 1,8-ANS fluorescence, three plates were prepared to measure such at TO, after 3FT cycles and following 2 days at 5000. Furthermore, objectives for this plate included to investigate other buffers, in- or excluding Tween® 80. The results after incubation for 1 day at 5000 are shown in table 8 showing data of the stability of 1 mg/ml antibody solutions in plate 5 before (T0) and after two days at 5000 (2 d50° C.).

TABLE 8
Results from Plate 5 after incubation for 2 days at 50° C.

| | | | | | | | Intrinsic | | | | |
|---|---|---|---|---|---|---|---|---|---|---|---|
| Antibody with 290 mM | | | | Turbidity | | | fluorescence | | 1,8-ANS | | |
| sucrose* and | | | | UV-350 nm | | | Em. | | | λ | |
| Buffer | | | | | 2d | | max | | | max | |
| (5 mM) | pH | Detergent | in % | T0 | 50° C. | % | % | Δ λ | % | Δ λ | CF |
| water | — | — | — | 0.047 | 0.125 | 166 | 67 | 2 | 2238 | −24 | 397 |
| NaS | 4 | T80 | 0.02 | 0.049 | 0.051 | 4 | 113 | 11 | 636 | −26 | 79 |
| NaS | 4 | T20 | 0.02 | 0.053 | 0.048 | −9 | 110 | 16 | 798 | 15 | 100 |
| NaS | 4 | T80 | 0.10 | 0.046 | 0.054 | 17 | 112 | 17 | 2732 | 16 | 301 |
| NaA | 4 | T80 | 0.02 | 0.048 | 0.052 | 8 | 99 | 3 | 621 | −10 | 80 |
| NaA | 4 | T20 | 0.02 | 0.047 | 0.051 | 9 | 89 | 0 | 707 | −11 | 89 |
| NaA | 4 | T80 | 0.10 | 0.048 | 0.052 | 8 | 91 | 4 | 2428 | −10 | 260 |
| NaP | 7 | T80 | 0.02 | 0.051 | 0.082 | 61 | 87 | −2 | 57 | −6 | 75 |
| NaP | 7 | T20 | 0.02 | 0.058 | 0.092 | 59 | 97 | −2 | 83 | −7 | 77 |
| NaP | 7 | T80 | 0.10 | 0.05 | 0.068 | 36 | 87 | −1 | 412 | −24 | 86 |
| CitP | 7 | T80 | 0.02 | 0.051 | 0.068 | 33 | 92 | −2 | 57 | −2 | 48 |
| CitP | 7 | T20 | 0.02 | 0.056 | 0.082 | 46 | 99 | −1 | 83 | −6 | 64 |
| CitP | 7 | T80 | 0.10 | 0.049 | 0.064 | 31 | 91 | 0 | 348 | −21 | 75 |
| Hist | 7 | T80 | 0.02 | 0.054 | 0.061 | 13 | 77 | 0 | 51 | 0 | 26 |
| Hist | 7 | T20 | 0.02 | 0.053 | 0.061 | 15 | 81 | −2 | 90 | −4 | 32 |
| Hist | 7 | T80 | 0.10 | 0.051 | 0.065 | 27 | 75 | −1 | 477 | −23 | 82 |

The results of Plate 5 suggest that Tween® 80 provided the best stability after freezing-thawing and incubation for 2 days at 5000 from the tested options, especially in a histidine-based buffer system. CF=Change Factor. Scanner aspect data is not presented since this criterion was not a differentiating factor in this experiment. *The sample indicated with “water” did not contain any sucrose.

### Example 6

Plate 6 for Example 6 was prepared similarly to the previous, meaning all formulations were prepared in a large enough volume to allow division over three plates and measurements of turbidity, intrinsic and 1,8-ANS fluorescence at Time 0, after 3FT cycles and after 2 days at 50° C.

Objectives of this plate included to repeat formulations that provided good results in Plate 5 with other buffers, with or without Tween® 80 and to compare sucrose with maltose in several formulations.

TABLE 9
Results from Plate 6 after incubation for 2 days at 50° C.

| | | | | | | | | Intrinsic | | | | |
|---|---|---|---|---|---|---|---|---|---|---|---|---|
| Antibody with 290 mM | | | | | Turbidity | | | fluorescence | | | | |
| sucrose or maltose | | | | | UV - 350 nm | | | Em. | | 1,8-ANS | | |
| Buffer | | Deter- | | | | 2 d | | max | | | λ max | |
| (5 mM) | pH | gent | in % | Sugar | T0 | 50° C. | % | % | Δ λ | % | Δ λ | CF |
| water | — | — | — | — | 0.044 | 0.297 | 575 | 83 | 31 | 3919 | −30 | 975 |
| Hist | 5.5 | — | — | Suc | 0.049 | 0.057 | 16 | 37 | 0 | 308 | −19 | 51 |
| Hist | 5.5 | T80 | 0.02 | Suc | 0.052 | 0.056 | 8 | 54 | −2 | 53 | −2 | 19 |
| Hist | 6.5 | — | — | Suc | 0.051 | 0.057 | 12 | 39 | 0 | 242 | −18 | 40 |
| Hist | 6.5 | T80 | 0.02 | Suc | 0.052 | 0.06 | 15 | 49 | −2 | 40 | −4 | 24 |
| Hist | 6.5 | T80 | 0.02 | Mal | 0.049 | 0.061 | 24 | 20 | 0 | 57 | 0 | 32 |
| NaP | 6 | — | — | Suc | 0.05 | 0.056 | 12 | 27 | 2 | 228 | −23 | 38 |
| NaP | 6 | T80 | 0.02 | Suc | 0.051 | 0.059 | 16 | 30 | −1 | 42 | −3 | 23 |
| NaP | 6 | T80 | 0.02 | Mal | 0.049 | 0.063 | 29 | 16 | −2 | 1 | −2 | 31 |
| CitP | 6 | — | — | Suc | 0.047 | 0.053 | 13 | 40 | −3 | 190 | −17 | 36 |
| CitP | 6 | T80 | 0.02 | Suc | 0.05 | 0.055 | 10 | 73 | −1 | 50 | 0 | 22 |
| CitP | 6 | T80 | 0.02 | Mal | 0.049 | 0.06 | 22 | 38 | 2 | 64 | −3 | 32 |
| NaA | 5.5 | — | — | Suc | 0.048 | 0.056 | 17 | 40 | 0 | 314 | −22 | 52 |
| NaA | 5.5 | T80 | 0.02 | Suc | 0.049 | 0.057 | 16 | 71 | −2 | 69 | −5 | 30 |

A strong increase of 1,8-ANS fluorescence and a shift of the maximum wavelength was observed in all formulations without Tween® 80. Formulations with maltose were less stable than with sucrose. A significant increase of absorbance at 350 nm was measured in the formulations with CaCl₂ (data not shown). Scanner aspect data is not presented since this criterion was not a differentiating factor among the samples in this experiment.

For Examples 7-9, a short list of formulations was selected and prepared with 20 mg/ml antibody for further investigation.

General Overview of High Throughput Screen of 20 mg/mL Antibody Formulations

Two plates with various formulations at 20 mg/ml antibody were prepared.

As such, the screened 20 mg/mL antibody formulations were distributed amongst two plates. General conclusions drawn from these experiments wherein samples were subjected to 3FT cycles and incubation periods of 30 mins at 60° C., 6 days at 400 C or 3 days at 500 C were that all formulations stable after 3FT, pH differences were observed due to the buffering capacity of the BsAb, lower absorbance at 350 nm was found with histidine at pH 5.5 and stability did not improve with higher buffer strength.

### Preparation and Analysis of Formulations

Antibody solutions were dialyzed and concentrations of stock solutions were analysed as set forth in Example 1, albeit starting with a different concentration of the starting material (7.74 mg/ml). Samples were prepared with formulations containing 20 mg/ml antibody using appropriate dilutions. After dialysis a concentration step was performed to be able to dilute antibody to 20 mg/ml with concentrated excipient solutions. In such cases, after dialysis, the antibody in water was concentrated at 25° C. using Amicon® Ultra centrifugal filter devices with a molecular weight cut-off of 10 kDa. The samples were centrifuged for 60 min at 4′000 rpm. The concentration was determined using the NanoDrop.

100 μl of each antibody formulation were dispensed in half-area UV-transparent 96-well Costar Corning plates and measured just after preparation of the formulations (Time 0). The plates were sealed with VIEWseal™ Transparent adhesive sealers before freezing/thawing or temperature stress.

Two plates with 15 antibody formulations at 20 mg/ml were prepared after dialysis and concentration of antibody in water. Each formulation was measured in duplicate.

Plate 7 for Example 7 was measured before and after 3 freeze-thaw cycles by Therapeomic scanner, UV-Vis absorbance and intrinsic fluorescence emission. After the 3 freeze-thaw cycles, the plate was incubated at 60° C. for 30 minutes and analyzed with the same methods.

Plate 8 for Example 8 was prepared in duplicate and measured by Therapeomic scanner, UV-Vis absorbance intrinsic and 1,8-ANS fluorescence emission: one plate was measured at Time 0 and the second plate was measured after 6 days at 40° C.; as 6 day 40° C. was not sufficient to degrade the protein the plate was then incubated at 50° C. and analyzed after 2 days and 3 days at 50° C. 1,8-ANS fluorescence was measured only after 3 days at 50° C.

To prepare the different formulations, concentrated buffer solutions were used. The pH of each formulation was measured after mixing of all ingredients with antibody. At 20 mg/ml significant differences were observed between the pH of the buffer stock solutions and the final pH, potentially indicating that antibody imparts its own self-buffering effect.

### Example 7

Objectives of Example 7 using Plate 7 included to determine if the best antibody formulations at 1 mg/ml are also stable after freezing-thawing and thermal stress at 20 mg/ml antibody, thereby bridging results from 1 mg/mL to 20 mg/mL and facilitating plausible extrapolations therefrom.

After the 3 freeze-thaw cycles, Plate 7 was incubated for 30 min at 60° C. The results after such incubation are shown in Table 10. Table 10 shows a data overview of the stability of antibody samples in plate 7 after three freeze-thaw cycles (3FT) and after 30 minutes at 60° C. (30 min 60° C.).

TABLE 10
Results of Plate 7 before and after incubation for 30 min at 60° C.

| Antibody with Tween ®80 and | | | | | | | Intrinsic | | | | | |
|---|---|---|---|---|---|---|---|---|---|---|---|---|
| 290 mM sucrose or maltose | | | | Turbidity | | | fluorescence | | | | Scanner | |
| | | Deter- | | UV - 350 nm | | | Emission max | | | | aspect | |
| Buffer | Final | gent | | | 30 mins | | | 30 mins | | λ max | | 30 mins |
| (5 mM) | pH | (in %) | Sugar | 3 FT | 60° C. | % | 3 FT | 60° C. | % | Δ λ | 3 FT | 60° C. |
| water | 7.7 | — | — | 0.177 | 1.863 | 953% | 41924 | 65161 | 55% | 26 | 3 | 3 |
| NaS | 6.8 | 0.02% | Suc | 0.079 | 0.259 | 228% | 40077 | 53345 | 33% | 2 | 1 | 3 |
| NaS | 6.7 | 0.10% | Suc | 0.078 | 0.23 | 195% | 40659 | 52632 | 29% | −1 | 1 | 3 |
| NaS | 6.7 | 0.02% | Mal | 0.075 | 0.235 | 213% | 39756 | 51457 | 29% | 0 | 1 | 3 |
| Hist | 6.3 | 0.02% | Suc | 0.066 | 0.067 | 2% | 38969 | 47853 | 23% | 1 | 1 | 1 |
| Hist | 6.3 | 0.02% | Mal | 0.064 | 0.066 | 3% | 39028 | 47548 | 22% | −2 | 1 | 1 |
| Hist | 7 | 0.02% | Suc | 0.073 | 0.123 | 68% | 38445 | 49540 | 29% | 1 | 1 | 2 |
| NaC | 6.8 | 0.02% | Suc | 0.077 | 0.112 | 45% | 38830 | 48860 | 26% | −1 | 1 | 2 |
| NaC | 6.8 | 0.02% | Mal | 0.077 | 0.128 | 66% | 38207 | 48867 | 28% | 0 | 1 | 2 |
| NaP | 6.9 | 0.02% | Suc | 0.076 | 0.355 | 367% | 37966 | 51823 | 36% | 2 | 1 | 3 |
| NaP | 6.9 | 0.02% | Mal | 0.079 | 0.386 | 389% | 39076 | 53426 | 37% | 0 | 1 | 3 |
| CitP | 6.9 | 0.02% | Suc | 0.082 | 0.2 | 144% | 38906 | 51120 | 31% | −1 | 1 | 3 |
| CitP | 6.9 | 0.02% | Mal | 0.083 | 0.201 | 142% | 38762 | 50782 | 31% | −1 | 1 | 3 |
| NaA | 7.1 | 0.02% | Suc | 0.081 | 0.266 | 228% | 38673 | 52326 | 35% | −2 | 1 | 3 |
| NaA | 7.1 | 0.02% | Mal | 0.081 | 0.256 | 216% | 37812 | 50403 | 33% | 0 | 2 | 3 |

The results of this plate suggest that for all formulations, the pH measured was higher than the pH of the buffer stock solutions (data not shown). These differences are likely due to the buffering capacity of the antibody at 20.0 mg/ml. All formulations appeared relatively stable after three freezing-thawing cycles. Histidine-based formulations with the lowest pH had low absorbances at 350 nm. Samples with a low pH histidine buffer remained stable after stress at 60° C., possibly due to the relatively lower pH of the formulations: 6.3 instead of 6.7-7.1. A pH of 5.5-6.5 would thus appear advantageous, especially a histidine buffer system. Interestingly, samples containing a histidine-based buffer gave the best outcomes in this experiment which was also found for samples containing 1 mg/ml antibody. Sodium citrate also performs well, even more so in combination with sucrose.

### Example 8

In this Example, plates were prepared in duplicate; all formulations were prepared in a large volume and divided over 2 plates. To determine the best stress conditions the second plate was stressed at 40° C. and then 50° C. Turbidity, intrinsic and 1,8-ANS fluorescence were measured for the 1^{st} plate was measured at Time 0. For the 2^{nd} plate, the following was measured at the indicated time points: turbidity and intrinsic fluorescence after 6 days at 40° C. and after 2 days at 50° C. (data not shown). Turbidity, intrinsic and 1,8-ANS fluorescence were measured after 6 days at 40° C. followed by 3 days at 50° C.

Objectives of Example 8 using Plate 8 included to increase buffer strength to investigate the buffering capacity of antibody and to better study the stability of formulations containing sucrose.

The results after 6 days at 40° C. followed by 3 days at 50° C. (3 d50° C.) are shown in Table 11. Table 11 shows a data overview of the stability of 20 mg/ml antibody solutions in plate 8 before (T0) and after 6 days at 40° C. followed by 3 days at 50° C. (3 d50° C.).

TABLE 11
Results of Plate 8 after 6 days at 40° C. followed by 3 days at 50° C.

| | | | | | | | Intrinsic | |
|---|---|---|---|---|---|---|---|---|
| Antibody with sucrose and | | | | Turbidity | | | fluorescence | |
| Tween ®80 | | | | UV - 350 nm | | | Emission | |
| Buffer | | T80 | Sucrose | | 3 d | | max | |
| (mM) | pH | (in %) | (mM) | T0 | 50° C. | % | % | Δ λ |
| Hist (5) | 6.3 | 0.02 | 290 | 0.062 | 0.074 | 19% | 31% | −1 |
| Hist (5) | 6.2 | 0.10 | 290 | 0.063 | 0.076 | 21% | 20% | −2 |
| Hist (5) | 7.0 | 0.02 | 290 | 0.071 | 0.105 | 48% | 29% | −1 |
| Hist (5) | 7.0 | 0.10 | 290 | 0.071 | 0.121 | 70% | 23% | −2 |
| Hist (10) | 5.9 | 0.02 | 280 | 0.063 | 0.081 | 29% | 30% | −2 |
| Hist (10) | 5.9 | 0.10 | 280 | 0.064 | 0.08 | 25% | 24% | −2 |
| Hist (10) | 6.8 | 0.02 | 280 | 0.066 | 0.103 | 56% | 32% | −1 |
| Hist (10) | 6.7 | 0.10 | 280 | 0.069 | 0.106 | 54% | 20% | −1 |
| Hist (15) | 5.8 | 0.02 | 270 | 0.066 | 0.084 | 27% | 36% | −1 |
| Hist (15) | 5.8 | 0.10 | 270 | 0.066 | 0.087 | 32% | 39% | −3 |
| Hist (15) | 6.6 | 0.02 | 270 | 0.073 | 0.115 | 58% | 32% | −1 |
| Hist (15) | 6.6 | 0.10 | 270 | 0.072 | 0.13 | 81% | 24% | −1 |
| NaC (15) | 6.8 | 0.02 | 270 | 0.083 | 0.09 | 8% | 37% | −3 |
| CitP (15) | 6.6 | 0.02 | 270 | 0.085 | 0.096 | 13% | 37% | −2 |
| NaS (15) | 6.0 | 0.02 | 270 | 0.080 | 0.098 | 23% | 37% | −1 |

Results of this plate suggested that higher buffer strengths do not improve the stability of the antibody, which implies that relatively low amounts of buffer are already sufficient to provide the desired effect. Absorbance at 350 nm was lower with Histidine buffer than with sodium citrate, citrate-phosphate and sodium succinate. Histidine formulations above about pH 6.5 were significantly less stable after thermal stress than at lower pH. A pH range of 5-6.5 of the antibody formulated in histidine buffer appears advantageous. Results obtained after incubation for 6 days at 40° C. and 2 days at 50° C. are not shown but were inline with results obtained after incubation for 6 days at 40° C. and 3 days at 50° C.

Sodium citrate-based formulations were among the most stable ones after three freeze-thaw cycles, while the histidine-based formulations are especially stable after being subjected to a further heat stress. Suitability of these formulations for in vivo studies and intravenous administration in human subjects was investigated next.

### Example 9. Compatibility Study of Selected Formulations for In Vivo Studies

Besides being formulated as a a stable and viable antibody composition, it is of importance the formulation is compatible with human plasma. Thus, compatibility of formulations 1-4 below with human plasma was investigated under two conditions: mixing the formulations with human and cynomulgus plasma. i) Plasma was mixed with the formulations diluted 5 times in 0.9% NaCl, which is a dilution that is representative of intravenous administration in human studies; and ii) plasma was mixed with undiluted formulations.
1) 20 mg/ml antibody, 5 mM Histidine pH 6.3, 290 mM Sucrose, 0.1% Tween® 80;
2) 20 mg/ml antibody, 10 mM Histidine pH 5.9, 280 mM Sucrose, 0.05% Tween® 80;
3) 20 mg/ml antibody, 10 mM Histidine pH 5.9, 280 mM Sucrose, 0.1% Tween® 80 and
4) 20 mg/ml antibody, 10 mM Na-Citrate pH 7.0, 280 mM Sucrose, 0.05% Tween® 80.

These four formulations were characterized for aggregation to occur in human plasma using 90° light-scatter and light-microscopy using the methods as described by Arvinte et al., (Aggregation of biopharmaceuticals in human plasma and human serum. Implication for drug research and development. In: mAbs 2013; 5:3, 491-500).

For the light microscopy method, 5 μl of antibody samples were placed inside a Quick-Read slide at 37° C. Then 3 μl of plasma was added inside the slide to allow contact with the protein solution as present; no mixing was performed. The microscopy slides were observed on a Leica DMi8 microscope (Leica Microsystems GmbH, Wetzlar, Germany) with a 5× objective and phase contrast. Pictures were recorded using a Leica MC170 HD camera. Samples were analyzed regarding their potential aggregation with plasma before and after dilution in 0.9% NaCl.

For the 90° light-scattering method, spectra were monitored at 37° C. using a FluoroMax-1 spectrofluorometer (HORIBA Jobin Yvon GmbH, Germany) by running a synchronous scan (λₑₘ=λₑₓ) between 500 nm and 750 nm with an integration time of 0.01 s in a 3 mm×3 mm black micro-cuvette. Excitation and emission slits of 1 mm were used. A 10% filter was placed at the excitation side to reduce the excitation signal intensity. To study potential aggregation with plasma by 90° light-scatter, the formulations were diluted 5 times with 0.9% NaCl (1 volume of protein for 4 volumes of 0.9% NaCl) and the plasma solutions were diluted 2 times with 0.9% NaCl. The cuvette was filled with 45 μl of the different antibody or buffer samples. The cuvette was equilibrated 5 min at 37° C. before addition and mixing of 5 μl of diluted plasma then the 90° light-scatter spectrum was immediately recorded.

All formulations that were 5 times diluted in 0.9% NaCl did not aggregate after mixing with human plasma from two donors nor with a pool of cynomolgus plasma.

Experiments with undiluted formulations with antibody and human and cynomolgus plasma samples showed that in formulations 1,2 and 3 slight, transient aggregation occurred at the interface. Aggregation occurred within seconds and disappeared rapidly and easily within 1 to 2 minutes. This effect was also seen for the buffer solutions of the three formulations, meaning aggregation is induced by the formulations and not by the antibody. No aggregation was observed for formulation 4 in the presence or absence of the antibody. The study thus shows compatibility of all four formulations with human plasma and their suitability for use in in vivo studies and intravenous administration in human subjects.

1-41. (canceled)

42. A pharmaceutical composition comprising:
a multispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
a buffer system comprising a histidine buffer, a citrate buffer, or both;
a sugar component; and
a non-ionic surfactant.

43. The pharmaceutical composition of claim 42, wherein the sugar component is or comprises sucrose.

44. The pharmaceutical composition of claim 42, wherein the non-ionic surfactant is or comprises polysorbate 80.

45. The pharmaceutical composition of claim 42, wherein the buffer system comprises a histidine buffer and the composition has a pH in the range of 5 to 7.

46. The pharmaceutical composition of claim 42, wherein the buffer system comprises a citrate buffer and the composition has a pH in the range of 6.2 to 7.8.

47. The pharmaceutical composition of claim 42, wherein the antibody is present at a concentration in the range of 0.5 to 150 mg/mL.

48. The pharmaceutical composition of claim 42, wherein the sugar component is present at a concentration in the range of 200 to 400 mM, or wherein the molar concentration ratio of sugar component to antibody is in the range of 250:1 to 60,000:1.

49. The pharmaceutical composition of claim 42, wherein the non-ionic surfactant is present at a concentration in the range of 0.01 to 2 mg/mL, or wherein the molar concentration ratio of non-ionic surfactant to antibody is in the range of 1:90 to 224:1.

50. The pharmaceutical composition of claim 42, wherein the buffer system is present at a concentration in the range of 2 to 50 mM, or wherein the molar concentration ratio of the buffer system to the antibody is in the range of 2.9:1 to 7,300:1.

51. The pharmaceutical composition of claim 42, wherein the composition comprises only a single buffer system, and wherein the single buffer system is a citrate or a histidine buffer system.

52. The pharmaceutical composition of claim 42, wherein the composition is free of trehalose.

53. The pharmaceutical composition of claim 42, wherein the composition is substantially free of one or more components selected from the group consisting of polysorbate 20, antioxidants, chelators, and non-buffering salt tonicifiers.

54. The pharmaceutical composition of claim 42, wherein the antibody is a bispecific antibody.

55. The pharmaceutical composition of claim 42, wherein the antibody comprises an EGFR binding domain comprising a heavy chain variable region (VH) that comprises the sequence of the VH chain of MF3370, MF3755, MF4280, or MF4289, or wherein the antibody comprises an LGR5 binding domain comprising a heavy chain variable region that comprises the sequence of the VH chain of MF5790, MF5803, MF5805, MF5808, MF5809, MF5814, MF5816, MF5817, or MF5818.

56. The pharmaceutical composition of claim 42, wherein the antibody comprises a heavy chain sequence corresponding to SEQ ID NO: 63, SEQ ID NO: 64, SEQ ID NO: 65, SEQ ID NO: 66, SEQ ID NO: 67, or SEQ ID NO: 68.

57. The pharmaceutical composition of claim 42, wherein the antibody comprises a binding domain comprising a light chain variable region (VL) that comprises the sequence of SEQ ID NO: 53.

58. The pharmaceutical composition of claim 42, wherein the antibody comprises a light chain constant region (CL) that comprises the sequence of SEQ ID NO: 57.

59. The pharmaceutical composition of claim 42, wherein the antibody comprises a variable heavy chain amino acid sequence of MF3370 as depicted in SEQ ID NO: 1, a variable heavy chain amino acid sequence of MF5790 as depicted in SEQ ID NO: 17, and a variable light chain amino acid sequence as depicted in SEQ ID NO: 53.

60. The pharmaceutical composition of claim 42, wherein the antibody comprises a variable heavy chain amino acid sequence of MF3755 as depicted in SEQ ID NO: 5, a variable heavy chain amino acid sequence of MF5816 as depicted in SEQ ID NO: 41, and a variable light chain amino acid sequence as depicted in SEQ ID NO: 53.

61. The pharmaceutical composition of claim 42, wherein the antibody comprises a variable heavy chain amino acid sequence of MF4280 as depicted in SEQ ID NO: 9, a variable heavy chain amino acid sequence of MF5809 as depicted in SEQ ID NO: 33, and a variable light chain amino acid sequence as depicted in SEQ ID NO: 53.

62. The pharmaceutical composition of claim 42, wherein the antibody comprises a variable heavy chain amino acid sequence of MF4289 as depicted in SEQ ID NO: 13, a variable heavy chain amino acid sequence of MF5818 as depicted in SEQ ID NO: 49, and a variable light chain amino acid sequence as depicted in SEQ ID NO: 53.

63. The pharmaceutical composition of claim 42, wherein the antibody comprises:
EGFR variable heavy chain MF3755 as depicted in SEQ ID NO: 5, MF3370 as depicted in SEQ ID NO: 1, MF4280 as depicted in SEQ ID NO: 9, or MF4289 as depicted in SEQ ID NO: 13;
LGR5 variable heavy chain MF5790 as depicted in SEQ ID NO: 17, MF5809 as depicted in SEQ ID NO: 33, MF5816 as depicted in SEQ ID NO: 41, or MF5818 as depicted in SEQ ID NO: 49;
variable light chain as depicted in SEQ ID NO: 53;
constant light chain as depicted in SEQ ID NO: 57;
the CH1 domain sequence as depicted in SEQ ID NO: 58;
the hinge sequence as depicted in SEQ ID NO: 59;
the CH2 domain sequence as depicted in SEQ ID NO: 60;
the CH3-DE domain sequence as depicted in SEQ ID NO: 61; and
the CH3-KK domain sequence as depicted in SEQ ID NO: 62.

64. A drug delivery device comprising the pharmaceutical composition of claim 42.

65. A pharmaceutical composition, wherein the composition is an aqueous composition comprising:
1-30 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
3-20 mM buffer system comprising a histidine buffer, a citrate buffer, or both;
200-400 mM sugar, sugar alcohol, or both; and
0.01-2 mg/mL non-ionic surfactant;
wherein the composition has a pH between 5.4 and 7.4.

66. A pharmaceutical composition, wherein the composition is an aqueous composition comprising:
15-25 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
4-12 mM buffer system comprising a histidine buffer, a citrate buffer, or both;
250-350 mM sucrose; and
0.1-1.5 mg/mL polysorbate 80;
wherein the composition has a pH between 5.4 and 7.4.

67. A pharmaceutical composition comprising:
20 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
10 mM histidine buffer system;
280 mM sucrose;
0.5 mg/mL polysorbate 80; and
water;
wherein the composition has a pH between 5.8 and 6.0.

68. A pharmaceutical composition comprising:
20 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
10 mM citrate buffer system;
280 mM sucrose;
0.5 mg/mL polysorbate 80; and
water;
wherein the composition has a pH between 6.8 and 7.2.

69. A pharmaceutical composition comprising:
20 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
5 mM histidine buffer system;
290 mM sucrose;
1.0 mg/mL polysorbate 80; and
water;
wherein the composition has a pH between 6.1 and 6.5.

70. A pharmaceutical composition comprising:
20 mg/mL bispecific antibody, functional part, or derivative thereof, comprising a first binding domain binding to Epidermal Growth Factor Receptor (EGFR) and a second binding domain binding to Leucine-rich repeat-containing G protein-coupled Receptor 5 (LGR5);
10 mM histidine buffer system;
280 mM sucrose;
1.0 mg/mL polysorbate 80; and
water;
wherein the composition has a pH between 5.8 and 6.0.
